(19) United States
(12) Patent Application Publication
(10) Pub. No.: US 2009/0099195 A1
(43) Pub. Date: Apr. 16, 2009

(54) Therapeutic Compounds 570

(75) Inventors: Malken Bayrakdarian, Saint Laurent (CA); Christophe Buon, Saint Laurent (CA); Louis-David Cantin, Saint Laurent (CA); Yun-Jin Hu, Saint Laurent (CA); Xuehong Luo, Saint Laurent (CA); Vijayaratnam Santhakumar, Saint Laurent (CA); Miroslaw Tomaszewski, Saint Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/115,169

(22) Filed: May 5, 2008

Related U.S. Application Data
(60) Provisional application No. 60/916,588, filed on May 8, 2007.

(51) Int. Cl. A61K 31/519; C07D 487/04; A61P 13/10; A61K 31/496

(52) U.S. Cl. 514/252.16; 544/280; 514/258.1

(57) ABSTRACT

Compounds of formula I or pharmaceutically acceptable salts thereof:

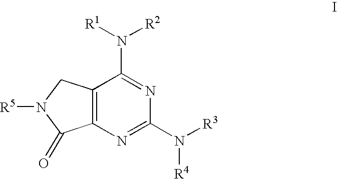

wherein R¹, R², R³, R⁴, and R⁵ and are as defined in the specification as well as salts and pharmaceutical compositions including the compounds are prepared. They are useful in therapy, in particular in the management of pain.

### BACKGROUND OF THE INVENTION

1. Field of the Invention

The invention is related to therapeutic compounds, pharmaceutical compositions containing these compounds, manufacturing processes thereof and uses thereof. Particularly, the present invention is related to compounds that may be effective in treating pain and/or over active bladder.

2. Discussion of Relevant Technology

The P2X purinoreceptors are a family of ion channels that are activated by extracellular adenosine triphosphate (ATP). Purinoreceptors have been implicated in a variety of biological functions, especially those related to pain sensitivity. The P2X3 receptor subunit is a member of this family that was originally cloned from rat dorsal root ganglia (Chen et al., Nature 1995, 377, 428-431). The nucleotide and amino acid sequences of both rat and human P2X3 are known (Lewis et al., Nature 1995, 377, 432-435; and Garcia-Guzman et al., Brain Res. Mol. Brain. Res. 1997, 47, 59-66). P2X3 is involved in afferent pathways controlling urinary bladder volume reflexes. Therefore, inhibiting P2X3 may have therapeutic potential in the treatment of disorders of urine storage and voiding such as overactive bladder (Cockayne et al., Nature 2000, 407, 1011-5). P2X3 also is selectively expressed on nociceptive, small diameter sensory neurons (i.e., neurons that are stimulated by pain or injury), consistent with a role in pain sensitivity. A method for reducing the level or activity of P2X3 therefore would be useful for modulating pain sensation in a subject suffering from chronic pain. P2X3 is also capable of forming P2X2/3 heterodimers with another member of the P2X purinergic ligand-gated ion channel family, P2×2. P2X2/3 is highly expressed on the terminals (central and peripheral) of sensory neurons (Chen et al., Nature 1995, 377, 428-431. Results from recent studies also suggest that P2X2/3 is predominantly expressed (over P2×3) in bladder sensory neurons and are likely to play an important role in sensing of urinary bladder filling and nociception (Zhong et al., Neuroscience 2003, 120, 667-675).

Therefore, there is a need for new P2X3 and/or P2X2/3 receptor ligands such as antagonists that may be useful in managing pain or treating other related symptoms or diseases.

### DESCRIPTION OF THE EMBODIMENTS

Certain embodiments of the present invention may be P2X3 receptor ligands which may be useful in treating pain and/or other related symptoms or diseases.

Certain compounds of the invention may be P2X2/3 receptor ligands which may be useful in treating pain and/or other related symptoms or diseases.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “alkynyl” used alone or as suffix or prefix, refers to a monovalent straight or branched chain hydrocarbon radical having at least one carbon-carbon triple bond and comprising at least 2 up to about 12 carbon atoms. The triple bond of an alkynyl group can be unconjugated or conjugated to another unsaturated group. Suitable alkynyl groups include, but are not limited to, C₂₋₆alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl can be unsubstituted or substituted with one or two suitable substituents.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to link two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “heteroaryl” used alone or as a suffix or prefix, refers to a heterocyclyl having aromatic character.

The term “heterocycloalkyl” used alone or as a suffix or prefix, refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, imidazolidine-2,4-dione, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, 10,11-dihydro-5H-benzo[4,5]cyclohepta[1,2-b]pyridine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy. The term “amine” or “amino” refers to —NH₂.

Halogen includes fluorine, chlorine, bromine and iodine.

“Halogenated,” used as a prefix of a group, means one or more hydrogens on the group are replaced with one or more halogens.

“RT”, “r.t.” or “rt” means room temperature.

In certain embodiments, one or more compounds of the present invention may exist as two or more diastereomers (also called “diastereo isomer”) or enantiomers. These two or more diastereo isomers or enantiomers may be isolated using one or more methods described in the invention or other known methods even though the absolute structures and configuration of these diastereo isomers or enantiomers may not be ascertained or determined. In order to identify and/or distinguish these diastereo isomers or enantiomers from each other, designations such as “isomer 1,” “isomer 2,” “diastereo isomer 1,” “diastereo isomer 2,” or “enantiomer 1,” “enantiomer 2” may be used to design the isolated isomers. One aspect of the invention is a compound of formula I, a pharmaceutically acceptable salt thereof, a diastereomer, an enantiomer, or a mixture thereof:

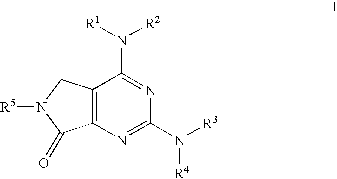

wherein:

R¹ and R² are independently selected from hydrogen, C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₃₋₇cycloalkyl fused with a phenyl and a C₂₋₆heteroaryl, C₆₋₁₀aryl fused with a C₃₋₇cycloalkyl, C₁₋₁₄heterocyclyl, C₁₋₁₄heterocyclyl-C₁₋₆alkyl, C₆₋₁₀aryl, C₆₋₁₀aryl-C₁₋₆alkyl, or R¹ and R² together with the nitrogen connected thereto form a C₂₋₉heterocyclyl; wherein said C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₃₋₇cycloalkyl fused with a phenyl and a C₂₋₆heteroaryl, C₆₋₁₀aryl fused with a C₃₋₇cycloalkyl, C₁₋₁₄heterocyclyl, C₁₋₁₄heterocyclyl-C₁₋₆alkyl, C₆₋₁₀aryl, C₆₋₁₀aryl-C₁₋₆alkyl and C₂₋₉heterocyclyl are optionally substituted by one or more groups selected from, halogen, cyano, nitro, C₁₋₆alkoxy, C₁₋₄haloalkoxy, C₁₋₆alkyl, halogenated C₁₋₆alkyl, C₃₋₆-cycloalkyl, C₃₋₆cycloalkoxy, C₃₋₆cycloalkyl-C₁₋₄alkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—R⁷, —(CH₂)ₘ—NR⁷R³, hydroxy, C₁₋₁₄heterocyclyl-C₀₋₄-alkyl, phenyl, benzyl, phenylethyl, wherein said C₁₋₁₄heterocyclyl-C₀₋₄-alkyl, phenyl, benzyl or phenethyl optionally substituted by one or more groups selected from halogen, cyano, nitro, oxo, C₁₋₆alkoxy, C₁₋₄haloalkoxy, C₁₋₆alkyl, halogenated C₁₋₆alkyl, C₃₋₆-cycloalkyl, C₃₋₆cycloalkoxy, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—R⁷, —(CH₂)ₘ—NR⁷R⁸ and hydroxy;

R³ and R⁴ are independently selected from hydrogen, C₁₋₆alkyl, C₂₋₆alkenyl, C₃₋₈cycloalkyl, C₃₋₈cycloalkyl-C₁₋₁₆alkyl, C₆₋₁₀aryl, C₆₋₁₀aryl-C₁₋₆alkyl, C₃₋₆heterocyclyl, and C₃₋₆heterocyclyl-C₁₋₆alkyl; or R³ and R⁴ together with the nitrogen connected thereto form a C₂₋₉heterocyclyl; wherein said C₁₋₆alkyl, C₂₋₆alkenyl, C₃₋₈cycloalkyl, C₃₋₈cycloalkyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl and C₂₋₉heterocyclyl are optionally substituted by one or more groups selected from C₁₋₆alkyl, halogenated C₁₋₆alkyl, carboxy, halogen, cyano, nitro, oxo, C₁₋₄-alkoxy, C₁₋₄haloalkoxy, hydroxy, C₃₋₆cycloalkyl-C₁₋₄alkoxy, C₃₋₆heterocycloalkyl, —(CH₂)ₘ—C₃₋₆heterocyclyl, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, (CH₂)ₘ—C(═O)R⁷, (CH₂)ₘ—S(═O)₂R⁷R⁸, —(CH₂)ₘ—O—C(═O)—R⁷, —(CH₂)ₘ—R⁷, and —(CH₂)ₘ—NR⁷R⁸;

R⁵ is selected from hydrogen and C₁₋₆alkyl, C₃₋₇-cycloalkyl, C₁₋₆heterocyclyl, and —(CH₂)ₘ—C₆₋₁₀aryl, optionally substituted with one or more groups selected from OH, C₁₋₄alkoxy, halogenated C₁₋₄alkoxy, and halogen;

R⁷ and R⁸ are independently selected from —H, C₁₋₆alkyl, C₆₋₁₀aryl, C₆₋₁₀aryl-C₁₋₄alkyl, C₁₋₅heterocyclyl, and C₃₋₆cycloalkyl-C₀₋₄alkyl, wherein said C₁₋₆alkyl, C₆₋₁₀aryl, C₆₋₁₀aryl-C₁₋₄alkyl, C₁₋₅heterocyclyl, and C₃₋₆cycloalkyl-C₀₋₄alkyl are optionally substituted with one or more groups selected from —OH, C₁₋₄alkyl, methoxy, ethoxy and halogen; and

m is 0, 1, 2 or 3,

with a proviso that at least one of R¹, R², R³ and R⁴ is not hydrogen

with a further proviso that the compound is not selected from 5-[2-(4-{4-[(4-chlorophenyl)amino]-6-isopropyl-7-oxo-6,7-dihydro-5H-pyrrolo[3,4-d]pyrimidin-2-yl}piperazin-1-yl)-2-oxoethyl]imidazolidine-2,4-dione 2-amino-4-anilino-5,6-dihydro-7H-pyrrolo[3,4-d]pyrimidin-7-one; 1-[4-[(4-chlorophenyl)amino]-6,7-dihydro-6-(1-methylethyl)-7-oxo-5H-pyrrolo[3,4-d]pyrimidin-2-yl]-4-[2-(hydroxyimino)-1-oxopropyl]-piperazine; 4-[(4-chlorophenyl)amino]-5,6-dihydro-2-(4-morpholinyl)-6-[2-(4-morpholinyl)ethyl]-7H-pyrrolo[3,4-d]pyrimidin-7-one; 4-[(2-aminophenyl)amino]-5,6-dihydro-5-imino-6-propyl-2-(1-pyrrolidinyl)-7H-pyrrolo[3,4-d]pyrimidin-7-one; 5,6-dihydro-5-imino-6-propyl-4-(propylamino)-2-(1-pyrrolidinyl)-7H-pyrrolo[3,4-d]pyrimidin-7-one; 5-amino-2-(1-piperidinyl)-4-(propylamino)-7H-pyrrolo[3,4-d]pyrimidin-7-one; 4,5-diamino-2-(1-piperidinyl)-7H-Pyrrolo[3,4-d]pyrimidin-7-one; 5-amino-4-(1-piperidinyl)-2-(1-pyrrolidinyl)-7H-pyrrolo[3,4-d]pyrimidin-7-one; 5-amino-4-(propylamino)-2-(1-pyrrolidinyl) 7H-pyrrolo[3,4-d]pyrimidin-7-one; 4,5-diamino-2-(1-pyrrolidinyl)-7H-pyrrolo[3,4-d]pyrimidin-7-one; N-[6,7-dihydro-7-oxo-6-phenyl-4-(1-piperidinyl)-5H-pyrrolo[3,4-d]pyrimidin-2-yl]-N-phenylbenzamide; 5-[(dimethylamino)methylene]-5,6-dihydro-6-phenyl-2-(phenylamino)-4-(1-piperidinyl)-7H-pyrrolo[3,4-d]pyrimidin-7-one; 2,4-bis(dimethylamino)-5,6-dihydro-6-phenyl-7H-pyrrolo[3,4-d]pyrimidin-7-one; 5,6-dihydro-6-phenyl-2-(phenylamino)-4-(1-piperidinyl)-7H-pyrrolo[3,4-d]pyrimidin-7-one; 5,6-dihydro-6-methyl-2,4-di-1-piperidinyl-7H-pyrrolo[3,4-d]pyrimidin-7-one; 4-anilino-6-butyl-2-(butylamino)-5,6-dihydro-7H-pyrrolo[3,4-d]pyrimidin-7-one; 4-anilino-2-p-anisidino-5,6-dihydro-6-(p-methoxyphenyl)-7H-pyrrolo[3,4-d]pyrimidin-7-one; 2-anilino-5,6-dihydro-4-(p-hydroxyanilino)-6-phenyl-7H-pyrrolo[3,4-d]pyrimidin-7-one; 2,4-dianilino-5,6-dihydro-6-phenyl-7H-pyrrolo[3,4-d]pyrimidin-7-one; 4-[(2-chloroethyl)amino]-5,6-dihydro-6-phenyl-2-piperidino-7H-pyrrolo[3,4-d]pyrimidin-7-one; 4-anilino-5,6-dihydro-6-(p-methoxyphenyl)-2-morpholino-7H-pyrrolo[3,4-d]pyrimidin-7-one; 4-anilino-5,6-dihydro-6-(p-methoxyphenyl)-2-piperidino-7H-pyrrolo[3,4-d]pyrimidin-7-one; 5,7-dihydro-2,4-dimorpholino-7-oxo-6H-Pyrrolo[3,4-d]pyrimidine-6-acetic acid 2-hydroxyethyl ester; 6-benzyl-5,6-dihydro-2,4-dimorpholino-7H-pyrrolo[3,4-d]pyrimidin-7-one; 5,6-dihydro-2,4-dimorpholino-6-phenyl-7H-pyrrolo[3,4-d]pyrimidin-7-one; 5,6-dihydro-6-(2-hydroxyethyl)-2,4-dipiperidino-7H-pyrrolo[3,4-d]pyrimidin-7-one; and 5,6-dihydro-6-phenyl-2,4-di-1-piperidinyl-7H-pyrrolo[3,4-d]pyrimidin-7-one.

In a particular embodiment, R¹ is hydrogen or C₁₋₄alkyl; and

R² is C₂₋₁₀heteroaryl-C₁₋₄alkyl, C₃₋₆heterocycloalkyl, or C₆₋₁₀aryl-C₁₋₄alkyl, wherein said C₂₋₁₀heteroaryl-C₁₋₄alkyl, C₃₋₆heterocycloalkyl, and C₆₋₁₀aryl-C₁₋₄alkyl are optionally substituted with one or more groups selected from halogen, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷—(CH₂)ₘ—SR⁷, —(CH₂)ₘ—O—C(═O)—R⁷, —(CH₂)ₘ—R⁷, —(CH₂)ₘ—NR⁷R⁸, hydroxy, phenyl, benzyl, phenylethyl, halogenated phenyl, halogenated benzyl and halogenated phenylethyl, wherein said phenyl, benzyl, phenylethyl, halogenated phenyl, halogenated benzyl and halogenated phenylethyl are optionally substituted with one or more groups selected from —OH, methoxy, ethoxy, halogenated C₁₋₆alkyl, and C₁₋₆alkyl, wherein said R⁷ and R⁸ are independently selected from —H, C₁₋₁₆alkyl, C₆₋₁₀aryl, C₁₋₁₅heterocyclyl, and C₃₋₆cycloalkyl-C₀₋₄alkyl, wherein said C₁₋₁₆alkyl, C₆₋₁₀aryl, C₁₋₅heterocyclyl, and C₃₋₆cycloalkyl-C₀₋₄alkyl used in defining R⁷ and R⁸ are optionally substituted with one or more groups selected from —OH, methoxy, ethoxy and halogen.

In another particular embodiment, R¹ is hydrogen or C₁₋₄alkyl; and R² is selected from cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicyclo[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl, wherein said cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl are optionally substituted with one or more groups selected from halogen, cyano, nitro, C₁₋₄alkoxy, C₁₋₆alkyl, halogenated C₁₋₆alkyl, C₃₋₆heterocycloalkyl, C₃₋₆cycloalkyl, C₃₋₆cycloalkyl-C₁₋₄alkoxy, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—OR⁷, —(CH₂)ₘ—NR⁷R⁸, hydroxy, phenyl, benzyl, phenylethyl, halogenated phenyl, halogenated benzyl and halogenated phenylethyl.

In an even further embodiment, R¹ is hydrogen or C₁₋₄alkyl; and R² is selected from cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl wherein said cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl are optionally substituted with one or more groups selected from hydroxyl, methoxy, ethoxy, methoxymethyl, cyclopropylmethoxy, halogenated C₁₋₃alkoxy, halogenated C₁₋₃alkyl, halogen, methyl, ethyl, isopropyl, phenyl, benzyl, phenylethyl, halogenated phenyl, halogenated benzyl and halogenated phenylethyl.

In a further embodiment, R¹ and R² together with the nitrogen connected thereto form a C₃₋₆heterocycloalkyl, wherein said C₃₋₆heterocycloalkyl is optionally substituted with one or more groups selected from halogen, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, (CH₂)ₘ—N(R⁷)C(═O)—OR⁸, (CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—OR⁷, —(CH₂)ₘ—NR⁷R⁸, hydroxy, C₂₋₉heterocyclyl, phenyl, benzyl, phenylethyl, halogenated phenyl, halogenated benzyl and halogenated phenylethyl, wherein said C₂₋₉heterocyclyl, phenyl, benzyl, phenylethyl, halogenated phenyl, halogenated benzyl and halogenated phenylethyl are optionally substituted with one or more groups selected from hydroxyl, methoxy, ethoxy, methoxymethyl, cyclopropylmethoxy, halogen, methyl, ethyl, phenyl, benzyl, phenylethyl, halogenated phenyl, halogenated benzyl and halogenated phenylethyl.

In an even further embodiment, R¹ and R² together with the nitrogen connected thereto form pyrrolidinyl, morpholinyl or azetidinyl, wherein said pyrrolidinyl, morpholinyl, and azetidinyl are optionally substituted with one or more groups selected from methoxy, ethoxy, halogen, methyl, ethyl, quinolinyl, isoquinolinyl, oxadiazolyl, phenyl, and benzyl; wherein said quinolinyl, isoquinolinyl, oxadiazolyl, phenyl, and benzyl groups may be optionally substituted from halogen, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—C(═O)—R⁷, —(CH₂)ₘ—OR⁷, —(CH₂)ₘ—NR⁷R⁸, hydroxy, phenyl, benzyl, phenylethyl, halogenated phenyl, halogenated benzyl and halogenated phenylethyl.

In a yet further embodiment, R³ is hydrogen and R⁴ is quinuclidinyl or C₁₋₄alkyl, wherein said quinuclidinyl and C₁₋₄alkyl are optionally substituted with one or more groups selected from methylsulfonyl, dimethylamino, methylamino, acetylamino, hydroxy, methoxy, ethoxy, halogen, methyl, ethyl, 2-oxopyrrolidin-1-yl, tetrahydrofuranyl, phenyl, halogenated phenyl, pyridyl, halogenated pyridyl, halogenated benzyl and benzyl.

In another particular embodiment, R³ and R⁴ together with the nitrogen connected thereto form a C₂₋₉heterocyclyl, wherein said C₂₋₉heterocyclyl is optionally substituted by one or more groups selected from C₁₋₆alkyl, halogenated C₁₋₆alkyl, halogen, methoxy, ethoxy, morpholinyl, hydroxy, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘ—N(R⁷)C(═O)R⁸, —(CH₂)ₘ—C(═O)R⁷, —(CH₂)ₘ—S(═O)₂R⁷, and —(CH₂)ₘ—NR⁷R⁸; wherein R⁷ and R⁸ are independently selected from —H, C₁₋₆alkyl, and C₃₋₆cycloalkyl-C₀₋₄alkyl; and m is 0, 1, 2 or 3.

In a further embodiment, R³ and R⁴ together with the nitrogen connected thereto form a group selected from piperazinyl, piperidinyl, hexahydro-oxazolo[3,4-a]pyrazin-3-one-7-yl, hexahydro-pyrrolo[1,2-a]pyrazin-2-yl, 3-oxopiperazin-1-yl, 1,4-diazepan-1-yl, 2,5-diazabicyclo[2.2.1]hept-2-yl, 6-oxohexahydropyrrolo[1,2-a]pyrazin-2(1H)-yl, 5,6-dihydro-[1,2,4]triazolo[4,3-a]pyrazin-7(8H)-yl, morpholinyl and pyrrolidinyl, wherein piperazinyl, piperidinyl, hexahydro-oxazolo[3,4-a]pyrazin-3-one-7-yl, hexahydro-pyrrolo[1,2-a]pyrazin-2-yl, 3-oxopiperazin-1-yl, 1,4-diazepan-1-yl, 2,5-diazabicyclo[2.2.1]hept-2-yl, 6-oxohexahydropyrrolo[1,2-a]pyrazin-2(1H)-yl, 5,6-dihydro-[1,2,4]triazolo[4,3-a]pyrazin-7(8H)-yl, morpholinyl and pyrrolidinyl are optionally substituted by one or more groups selected from methyl, ethyl, isopropyl, cyclopropyl, acetyl, cyclopropylcarbonyl, isopropylcarbonyl, ethylcarbonyl, acetylamino, methylsulfonyl, and diethylaminocarbonylmethyl.

In an even further embodiment, R⁵ is n-propyl or isopropyl.

In another embodiment, each R⁷ and R⁸ are independently selected from —H, C₁₋₆alkyl, C₆₋₁₀aryl, C₁₋₅heterocyclyl, and C₃₋₆cycloalkyl-C₀₋₄alkyl, wherein said C₁₋₆alkyl, C₆₋₁₀aryl, C₁₋₅heterocyclyl, and C₃₋₆cycloalkyl-C₀₋₄alkyl are optionally substituted with one or more groups selected from —OH, methoxy, ethoxy and halogen.

In a further embodiment, each R⁷ and R⁸ are independently selected from —H and C₁₋₆alkyl.

In a further embodiment, m is 0.

In another embodiment, m is 1.

In a further embodiment, m is 2.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

It will also be understood that certain compounds of the present invention may exist in solvated, for example hydrated, as well as unsolvated forms. It will further be understood that the present invention encompasses all such solvated forms of the compounds of the formula I.

Within the scope of the invention are also salts of the compounds of the formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as ligands such as antagonists of P2X3 receptors. More particularly, the compounds of the invention are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. In addition, the compounds of the present invention may be useful in treating over active bladder. Furthermore, the compounds of the invention may be used to treat cancer, multiple sclerosis, Parkinson's disease, Huntington's chorea, Alzheimer's disease, anxiety disorders, gastrointestinal disorders and cardiovascular disorders.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of cannabinoid receptors is present or implicated in that paradigm. This may involve the use of isotopically labeled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung edema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anesthesia and monitored anesthesia care. Combinations of agents with different properties are often used to achieve a balance of effects needed to maintain the anesthetic state (e.g. amnesia, analgesia, muscle relaxation and sedation). Included in this combination are inhaled anesthetics, hypnotics, anxiolytics, neuromuscular blockers and opioids.

Also within the scope of the present invention is the use of any of the compounds according to the Formula I above, for the manufacture of a medicament for the treatment of any of the conditions discussed above.

A further aspect of the invention is a method for the treatment of a subject suffering from any of the conditions discussed above, whereby an effective amount of a compound according to the formula I above, is administered to a patient in need of such treatment.

Thus, the invention provides a compound of formula I, or pharmaceutically acceptable salt or solvate thereof, as hereinbefore defined for use in therapy.

In a further aspect, the present invention provides the use of a compound of formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substance, which may also act as diluents, flavoring agents, solubilizers, lubricants, suspending agents, binders, or tablet-disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized molds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I as defined above for the manufacture of a medicament.

Also within the scope of the invention is the use of any compound of formula I for the manufacture of a medicament for the therapy of pain and/or urinary tract disorders.

Additionally provided is the use of any compound according to Formula I for the manufacture of a medicament for the therapy of various pain conditions including, but not limited to: acute pain, chronic pain, neuropathic pain, back pain, cancer pain, and visceral pain.

Additionally provided is the use of any compound according to Formula I for the manufacture of a medicament for the therapy of various urinary tract disorders, including, but not limited to, over active bladder pelvic hypersensivity and urethritis.

A further aspect of the invention is a method for therapy of a subject suffering from any of the conditions discussed above, whereby an effective amount of a compound according to the formula I above, is administered to a patient in need of such therapies.

Additionally, there is provided a pharmaceutical composition comprising a compound of Formula I, or a pharmaceutically acceptable salt thereof, in association with a pharmaceutically acceptable carrier.

Particularly, there is provided a pharmaceutical composition comprising a compound of Formula I, or a pharmaceutically acceptable salt thereof, in association with a pharmaceutically acceptable carrier for therapy, more particularly for therapies of pain and urinary tract disorders.

Further, there is provided a pharmaceutical composition comprising a compound of Formula I, or a pharmaceutically acceptable salt thereof, in association with a pharmaceutically acceptable carrier use in any of the conditions discussed above.

In a further embodiment, a compound of the present invention, or a pharmaceutical composition or formulation comprising a compound of the present invention may be administered concurrently, simultaneously, sequentially or separately with one or more pharmaceutically active compound(s) selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including for example quetiapine and pharmaceutically active isomer(s) and metabolite(s) thereof, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof;

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, NXY-059, citicoline, crobenetine, desmoteplase, repinotan, traxoprodil and equivalents and pharmaceutically active isomer(s) and metabolite(s) thereof;

(x) over active bladder urinary incontinence therapies including, for example, darafenacin, falvoxate, oxybutynin, propiverine, robalzotan, solifenacin, tolterodine and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents and pharmaceutically active isomer(s) and metabolite(s) thereof, and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combinations employ the compounds of this invention within the dosage range described herein and the other pharmaceutically active compound or compounds within approved dosage ranges and/or the dosage described in the publication reference.

In an even further embodiment, a compound of the present invention, or a pharmaceutical composition or formulation comprising a compound of the present invention may be administered concurrently, simultaneously, sequentially or separately with one or more pharmaceutically active compound(s) selected from buprenorphine; dezocine; diacetylmorphine; fentanyl; levomethadyl acetate; meptazinol; morphine; oxycodone; oxymorphone; remifentanil; sufentanil; and tramadol.

In a particular embodiment, it may be particularly effective to administrate a combination containing a compound of the invention and a second active compound selected from buprenorphine; dezocine; diacetylmorphine; fentanyl; levomethadyl acetate; meptazinol; morphine; oxycodone; oxymorphone; remifentanil; sufentanil; and tramadol to treat chronic nociceptive pain.

Another aspect of the invention is a method of preparing the compounds of the present invention.

In one embodiment, the invention provides a method for preparing a compound of formula I,

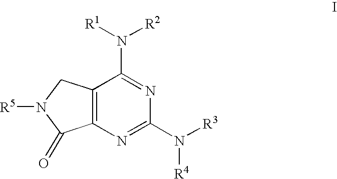

comprising reacting a compound of formula II with HNR³R⁴

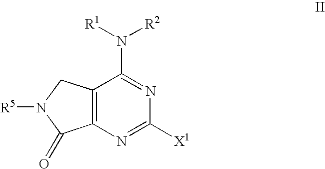

wherein:

X¹ is halogen; and R¹, R², R³, R⁴, and R⁵ are as defined above.

In another embodiment, the method of making a compound of formula I described above is carried out at a temperature between 100° C.-200° C., optionally in the presence of a microwave heating source, optionally in the presence of a solvent such as n-butanol.

In another embodiment, the invention provides a method for preparing a compound of formula II,

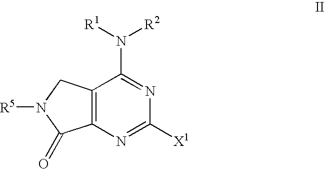

comprising reacting a compound of formula III with HNR¹R²

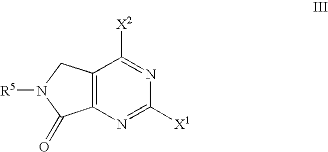

wherein:

X¹ and X² are independently halogens; and R¹, R², and R⁵ are defined as above.

In a further embodiment, the method of making a compound of formula II described above is carried out at a temperature between rt and 100° C., optionally in the presence of an organic base such as triethylamine or diisopropylethylamine, and further optionally in the presence of a solvent such as dichloromethane or t-butanol.

Compounds of the present invention may be prepared according to the synthetic routes as depicted in Schemes 1-16 using the Following a procedure similar to that described in General procedures.

### General Synthetic Methods

In one embodiment, the invention provides a process for preparing the compounds of Formula I, starting from Formula 1.1, according to the methods described below, where R¹ through R⁵ are defined in Formula I:

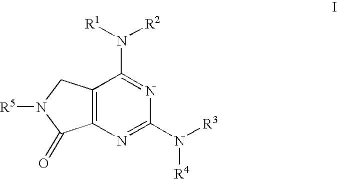

In Scheme 1, compounds of Formula I are prepared by the displacement of dichloropyrimidines of Formula 1.1 sequentially using either a primary or a secondary amine in each of the steps. This reaction can be performed in one pot under various conditions. For example, by reacting dichloropyrimidines of Formula 1.1 with amines 1.2 in a polar or non-polar solvent such as 1,2-dichloroethane, DCM, n-BuOH, t-BuOH, i-PrOH, in the presence of a base such as TEA or DIPEA yields the mono-displaced intermediates, which can be further reacted with amines 1.3 in a polar solvent such as n-BuOH, t-BuOH, i-PrOH in the presence of a base such as DIPEA to yield compounds of Formula I. The reaction temperature for the first displacement can range from 0° C. up to 160° C. For the second displacement, temperatures ranging from 130° C. up to 170° C. are preferred. Both conventional heating and microwave irradiation can be used.

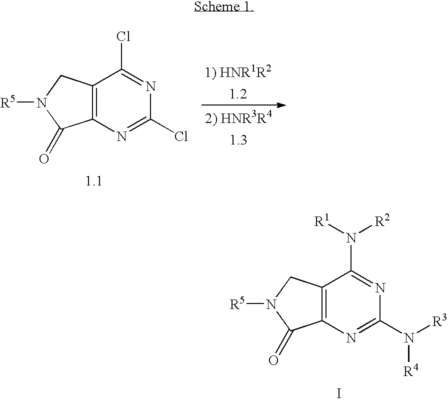

Alternatively, as illustrated in Scheme 2, compounds of Formula I can be prepared following the description given for Scheme 1, but with isolation and purification of intermediate 2.1 prior to the introduction of amines 1.3.

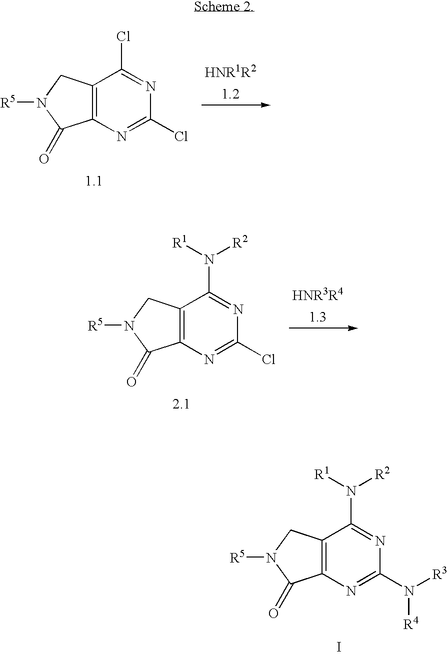

In another embodiment, compounds of Formula I can be prepared starting from a lactone (Formula 3.1) that is treated with amines 1.2 as illustrated in Scheme 3. The reaction can be carried out in a suitable solvent such as dichloromethane at temperatures ranging from 0° C. to 30° C., in the presence of a base such as DIPEA or Et₃N. The resulting amino derivative of Formula 3.2 can be reacted with amines 1.3 in a suitable protic solvent such as n-BuOH with heating (130-150° C.) in a sealed vessel such that the internal pressure is allowed to rise above 1 atm. The resulting diamino derivative of Formula 3.3 can be reacted with an amine 3.4 in the presence of a mineral acid such as HCl. The reaction can be carried out in a suitable solvent such as 2-methoxyethanol, with heating (160-200° C.) in a sealed vessel such that the internal pressure is allowed to rise above 1 atm.

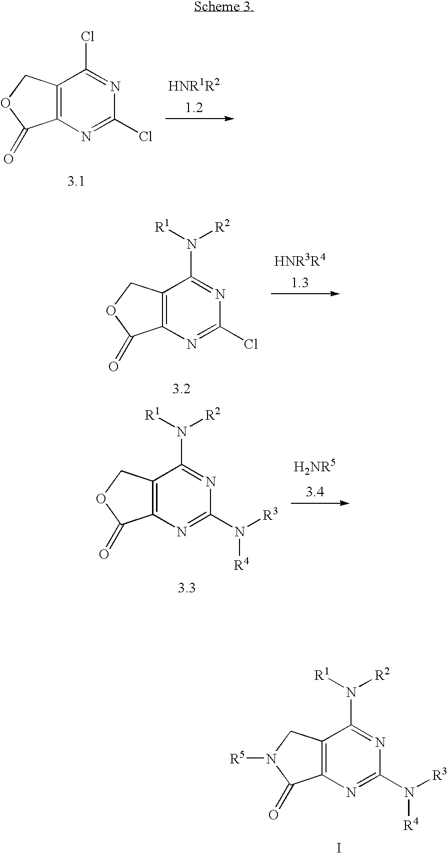

Scheme 4 illustrates the synthesis of compounds of Formula Ib, where NR³R⁴ is an hexahydro-oxazolo-[3,4-a]pyrazinone, by treatment of compounds of Formula Ia, where NR³R⁴ is a 3-hydroxymethylpiperazine, with a carbonate source such as phosgene, diphosgene, triphosgene or carbonyldiimidazole, in a suitable solvent such as dichloromethane, optionally in the presence of a base such as triethylamine or DIPEA.

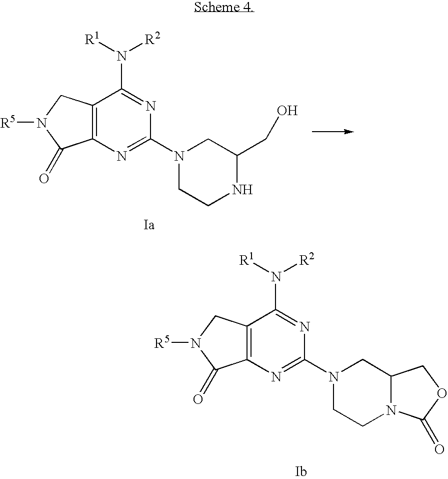

Scheme 5 illustrates the synthesis of compounds of Formula Ie, where NR³R⁴ is an optionally substituted acyl piperazine. Compounds of Formula 1c, synthesized as illustrated in Schemes 1, 2 or 3, can be treated with an acid, such as HCl or TFA, in a suitable solvent such as dichloromethane, 1,4-dioxane or THF to provide compounds of Formula 1d, where NR³R⁴ is an optionally substituted piperazine. Treatment of compounds of Formula Id with acylating agents, such as anhydrides or acyl chlorides, optionally in the presence of a mild base, such as Et₃N or DIPEA, in a suitable solvent such as dichloromethane can lead to compounds of Formula Ie where NR³R⁴ is an optionally substituted piperazine. Treatment of compounds of Formula Id where R¹=H and R² is an 3-methylene-isoquinoline, with an acylating agent as described above, can lead to bis-acylated products of Formula If.

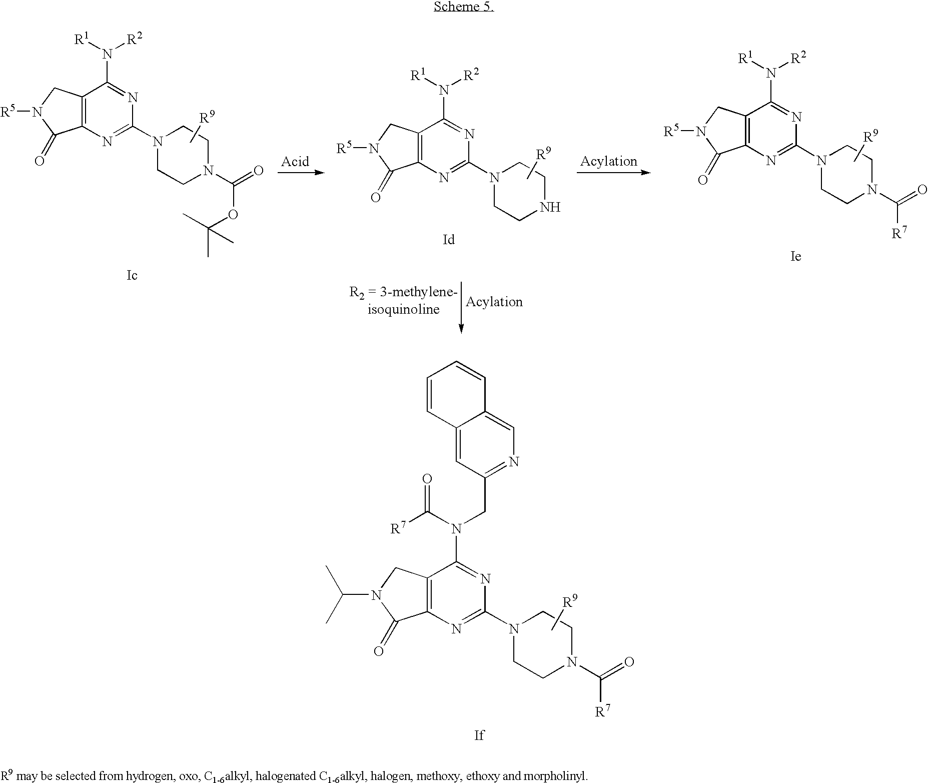

The synthesis of compounds of Formula Ih, where NR¹R² come together to form a pyrrolidine bearing an amide substituent, and of Formula Ii, where NR¹R² come together to form a pyrrolidine bearing an ester substituent, is described in Scheme 6. Acids of Formula Ig can be reacted with amines 6.1 under peptide coupling conditions, such as HOBT/DCC, HOAT/HATU, HOBT/HBTU, EDCI, in the presence of a base such as DIPEA or Et₃N in a suitable solvent such as THF, DMF or dichloromethane to provide compounds of Formula Ih. Acids of Formula Ig can also be reacted with alcohols 6.2 under standard peptide coupling conditions, such as HOBT/DCC, HOAT/HATU, HOBT/HBTU, EDCI, in the presence of a base such as DIPEA or Et₃N in a suitable solvent such as THF, DMF or dichloromethane to provide compounds of Formula Ii.

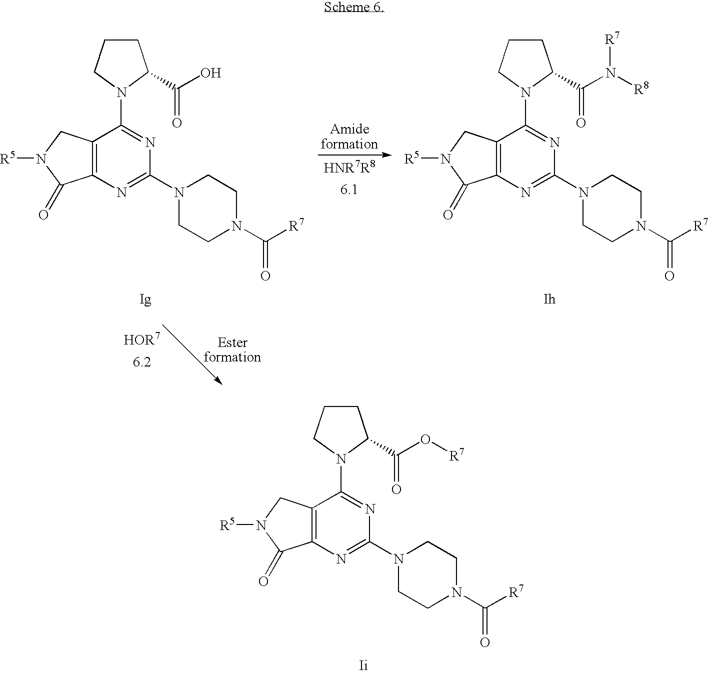

Scheme 7 illustrates the synthesis of intermediates of Formula 1.1 starting from orotic acid 7.1 with treatment with paraformadelhyde in the presence of a mineral acid, such as HCl. The reaction can be heated to temperatures ranging from 80 to 100° C., to lead to a dihydroxypyrimidine derivative 7.2. Further reaction of intermediate 7.2 with an amine 3.4, either as the HCl salt or as the free-base in the presence of one equivalent of a mineral acid, in a suitable solvent such as 2-methoxyethanol with heating at temperatures ranging from 190 to 200° C., can provide dihydroxypyrrolopyrimidines of Formula 7.4. An alternate synthetic route leading to intermediates of Formula 7.4 involves treatment of orotic acid under Mannich-type conditions to afford amino acid derivatives of Formula 7.3. The reaction is preferably carried out utilizing paraformaldehyde and an amine 3.4 in the presence of a mineral acid such as HCl. The reaction can be performed in a suitable solvent, such as ethanol, with heating at temperatures ranging from 60-80° C. Subsequent treatment of intermediate 7.3 with a concentrated mineral acid, such as HCl, in a suitable solvent such as 2-methoxyethanol can provide dihydroxypyrrolopyrimidines of Formula 7.4. In turn, dichloropyridmine derivatives of Formula 1.1 can be prepared by treating intermediates 7.4 with an halogenating agent, such as SOCl₂ or POCl₃, with or without a suitable solvent, such as dichlorethane, with heating at temperatures ranging from 70-90° C. The addition of a mild base, such as diethylaniline, can also be beneficial.

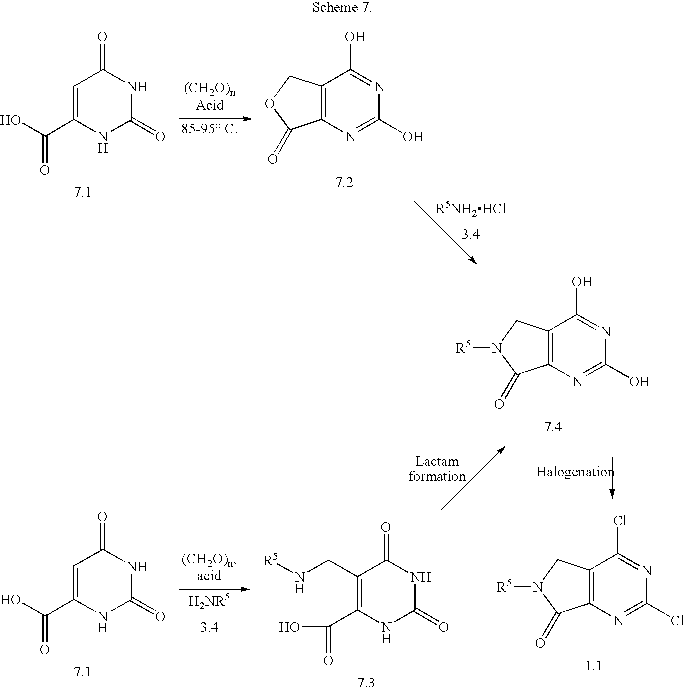

The synthesis of dichloropyrimidine derivatives of Formula 3.1 can be prepared by treating intermediates 7.2 with an halogenating agent, such as SOCl₂ or POCl₃, with or without a suitable solvent, such as dichloroethane, with heating at temperatures ranging from 70-90° C., as illustrated in Scheme 8. The addition of a mild base, such as diethylaniline, can also be beneficial.

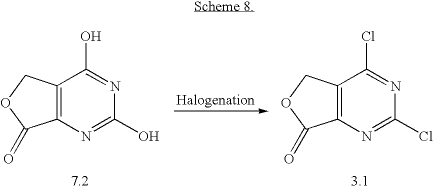

The synthesis of amines of Formula 1.2a, where R² bears an alpha-methyl group, can be achieved as illustrated in Scheme 9. (Ref: Liu, G.; Cogan, D. A.; Owens, T. D.; Tang, T. P.; Ellman, J. A. J. Org. Chem. 1999, 64, 1278-1284; Cogan, D. A.; Liu, G.; Ellman, J. A. Tetrahedron 1999, 55, 8883-8904). Condensation of aldehydes of Formula 9.1, obtained from commercial sources or synthesized using methods known to one skilled in the art, with sulfoximine 9.2 can be performed in a suitable solvent, such as dichloromethane, in the presence of a catalytic amount of acid, such as PTSA, and of a desiccant such as magnesium sulfate. The resulting sulfoximines of Formula 9.3 can be treated with a methyl-Grignard reagent in a suitable solvent, such as butyl ether, at temperatures ranging from −40° C. to 25° C. The resulting sulfinamide 9.4 can then be treated with an anhydrous mineral acid, such as HCl in 1,4-dioxane to provide amines of Formula 1.2a, where R² is CH(Me)-R⁷. When the starting sulfoximine 9.2 is enantioenriched, intermediates 9.4 can be obtained in a diastereoselective manner, leading to enantioenriched amines 1.2a.

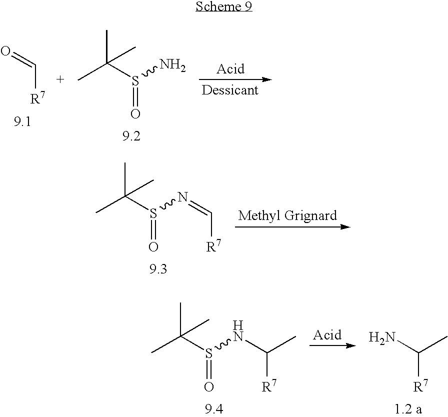

The synthesis of amines intermediates of structure 1.2b, where R² is a gem-dimethyl-CH₂R⁷, can be achieved starting from ketones 10.1 using methyl-Grignard in a suitable solvent such as ether or THF, as illustrated in Scheme 10. The resulting alcohols of structure 10.2 can be dissolved in acetic acid, and treated with acetonitrile in the presence of a mineral acid such as sulfuric acid (Timberlake, Jack W et al., Journal of Organic Chemistry 1981, 46, 2082-9). The resulting amides of Formula 10.3 can then be treated with a mineral acid such as HCl with heating at temperatures ranging from 90 to 100° C. to provide amines of Formula 1.2b.

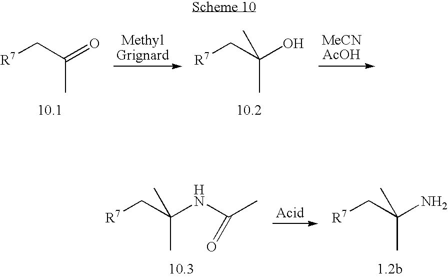

Scheme 11 illustrates the synthesis of amines of Formula 1.2c, where R¹ and R² come together to form a pyrrolidine ring substituted by a benzyl group. Starting from tert-butyl pent-4-enylcarbamate (Wolfe, J. P., et al., Tetrahedron 2005, 61(26), 6447-6459) which can be treated with an aryl bromide 11.2 in the presence of a catalytic amount of palladium (TI), preferably Pd(OAc)₂, with a phosphine-based ligand, such as 2,2′-oxybis(2,1-phenylene)bis(diphenylphosphine). The addition of a carbonate base, such as cesium carbonate, can be beneficial to the reaction. The reaction is preferably performed in a solvent, such as 1,4-dioxane, with heating to temperatures ranging from 140-160° C. in a microwave reactor to provide compounds of Formula 11.3. Compounds of Formula 11.3 can be treated with an acid, such as HCl or TFA, in a suitable solvent such as dichloromethane, 1,4-dioxane or THF to provide compounds of Formula 1.2c, isolated either as the free-base or the salt. R⁶ of Scheme 11 may be selected from fluoro, chloro, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷—(CH₂)ₘ—SR⁷, —(CH₂)ₘ—O—C(═O)—R⁷, —(CH₂)ₘ—R⁷, —(CH₂)ₘ—NR⁷R⁸, hydroxy, phenyl, benzyl, phenylethyl, fluorophenyl, chlorophenyl, fluorobenzyl, chlorobenzyl, fluorophenetyl and chlorophenylethyl, wherein said phenyl, benzyl, phenylethyl, fluorophenyl, chlorophenyl, fluorobenzyl, chlorobenzyl, fluorophenetyl and chlorophenylethyl, are optionally substituted with one or more groups selected from —OH, methoxy, ethoxy, halogenated C₁₋₆alkyl, and C₁₋₆alkyl, wherein said R⁷ and R⁸ are independently selected from —H, C₁₋₆alkyl, C₆₋₁₀aryl, C₁₋₅heterocyclyl, and C₃₋₆cycloalkyl-C₀₋₄alkyl, wherein said C₁₋₆alkyl, C₆₋₁₀aryl, C₁₋₅heterocyclyl, and C₃₋₆cycloalkyl-C₀₋₄alkyl used in defining R⁷ and R⁸ are optionally substituted with one or more groups selected from —OH, methoxy, ethoxy, fluoro and chloro.

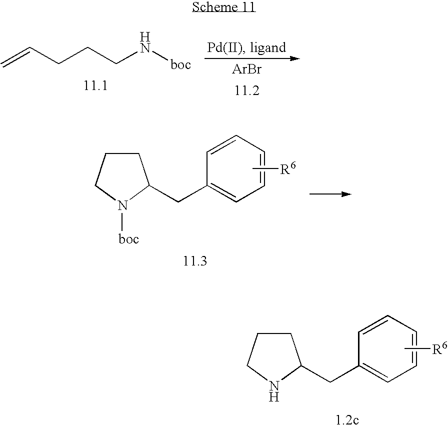

The synthesis of amines of Formula 1.2d, where R¹ is an optionally substituted alkyl group and R² is a substituted benzyl group can be synthesized through reductive amination, as illustrated in Scheme 12. Treatment of amine of Formula 12.1 with aldehydes of Formula 9.1 in the presence of zinc chloride and a suitable reducing agent such as sodium cyanoborohydride. The reaction is preferably performed in a protic solvent such as methanol to yield amines of Formula 1.2d.

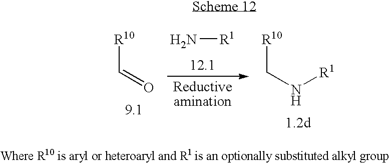

The synthesis of amines of Formula 1.2e and 1.2f, can be achieved starting form esters of Formula 13.1 that can be treated with a reducing agent, such as LiAlH₄, LiBH₄ or DIBAL, in a suitable solvent, as illustrated in Scheme 13. The resulting alcohols of Formula 13.2 can then be converted to the corresponding halides of Formula 13.3 using reagents such as SOCl₂, CCl₄/PPh₃ or Br₄/PPh₃ in a suitable solvent. Halides of Formula 13.3 can be reacted with primary amines of Formula 11.2 to yield amines of Formula 1.2e. Alternatively, halides of Formula 13.3 can be reacted with an azide salt, such as sodium azide, in a suitable polar solvent, such as DMF, optionally in the presence of potassium iodide to yield azides of Formula 13.4. In turn, azides of Formula 13.4 can be reduced, preferably using PPh₃ in THF in the presence of water, to provide primary amines of Formula 1.2f. Protection of primary amines 1.2f, preferably as Boc carmatates, can be achieved by treatment with di-tert-butyl dicarbonate in a mixture of a protic solvent, such as ethanol, and a dilute aqueous solution of NaHCO₃. The resulting carbamates of Formula 13.5 can then be treated with a strong base such as sodium hydride, and then exposed to an alkyl halide or alkyl sulfonate of Formula 13.6 in a suitable solvent such as ether or THF. The resulting alkyl carbamate 13.7 can be treated with an acid, such as HCl or TFA, in a suitable solvent such as dichloromethane, 1,4-dioxane or THF to provide compounds of Formula 1.2e.

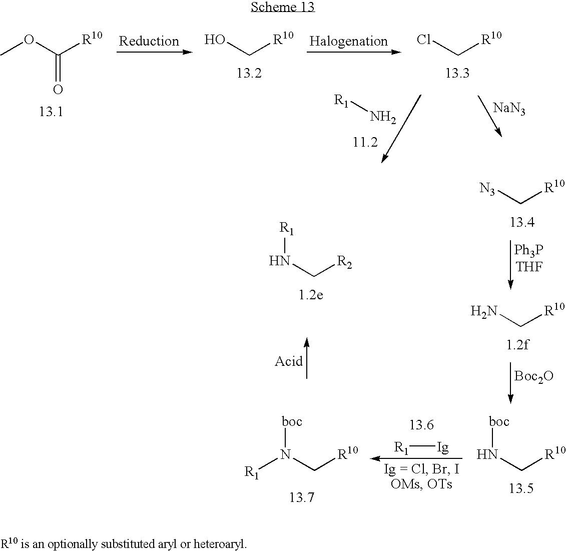

The synthesis of alcohols of Formula 13.2a, where R² is a 1-amino-3-methyleneisoquinoline, can alternatively be achieved starting form a dichloroisoquinoline 14.1 that can be exposed to an alkylamine 6.1 in a suitable solvent such as n-butanol, as illustrated in Scheme 14. The reaction is preferably performed with heating in a microwave reaction to provide monochloro derivatives 14.2. Treatment of 14.2 with zinc cyanide in the presence of a catalytic amount of palladium, such as Pd₂(dba)₃, and of a phosphine-based ligand, such as 1,1′-bis(diphenylphosphino)ferrocene (dppf), and of zinc dust. The reaction is preferably performing in a solvent such as DME, in the presence of 5-25% water. The reaction can be heated to temperatures ranging from 120-140° C. in a microwave reactor. The resulting nitrile 14.3 can be hydrolyzed utilizing a strong mineral acid, such as HCl, with heating at temperatures ranging from 90-100° C. to provide acids 14.4, which can be reduced to the corresponding alcohols 13.2a using a reducing agent, such as LiAlH₄ or BH₃, in a suitable solvent, such as ether or THF.

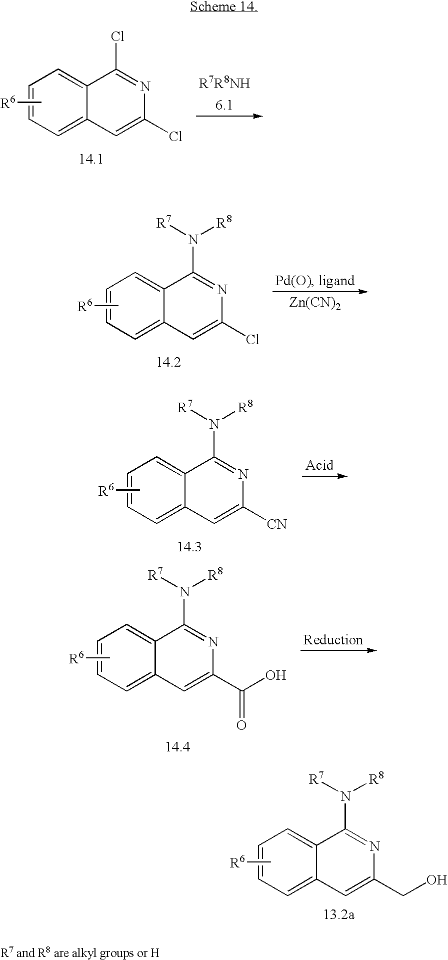

Scheme 14b illustrates the synthesis of primary amines of Formula 1.2 g by treatment of dichloroisoquinoline 14.1 with alkyl zinc chloride in the presence of a catalytic amount of palladium (0) and of a phosphine-based ligand, preferably their complex, such as tetrakis(triphenylphosphine)palladium(0). The reaction is preferably performed in a solvent such as THF. The reaction can be heated to temperatures ranging from 40-80° C. in a microwave reactor. The resulting alkyl isoquinoline 14.5 can be further converted to nitrile intermediate 14.6, following a similar procedure as described in Scheme 14. In turn, nitrile 14.6 can be reduced to primary amine 1.2 g by hydrogenation in the presence of a catalyst, such as dihydroxypalladium, and in a suitable solvent such as ethanol.

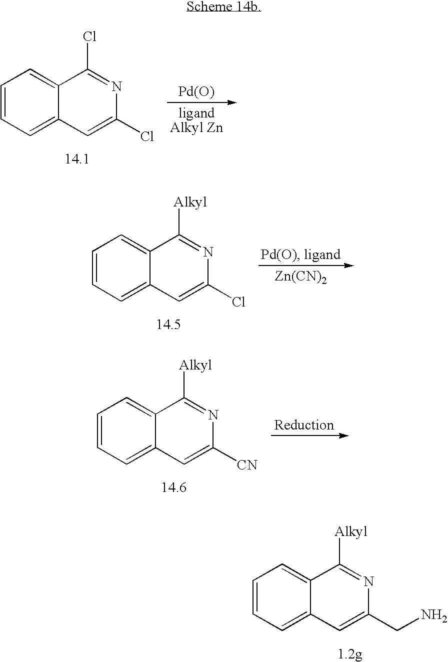

An alternative synthesis of alcohols of Formula 13.2b is illustrated in Scheme 15. Starting from derivatives of phenylalanine 15.1, treatment with formaldehyde in the presence of an acid, preferably HBr, with heating to temperatures ranging from 140-160° C. in a microwave reactor followed by esterification can provide tetrahydroisoquinolines of Formula 15.2. Subsequent oxidation by heating 15.2 in a suitable solvent, such as DMF, in the presence of a base, preferably DIPEA, to temperatures ranging from 90-120° C., can provide isoquinoline derivatives of Formula 15.3. Esters of Formula 15.3 can be treated with a reducing agent, such as LiAlH₄, LiBH₄ or DIBAL, in a suitable solvent such as THF to provide alcohols of Formula 13.2b. R⁶ of Scheme 15 may be selected from halogen, cyano, C₁₋₄alkoxy, C₁₋₆alkyl, halogenated C₁₋₆alkyl, C₃₋₆cycloalkyl, C₃₋₆cycloalkoxy, —(CH₂)ₘ—S(═O)₂NR⁷R⁸, —(CH₂)ₘNH—C(═O)NR⁷R⁸, —(CH₂)ₘ—S(═O)₂R⁷, —(CH₂)ₘ—S(═O)R⁷, —(CH₂)ₘ—SR⁷, —(CH₂)ₘ—R⁷, —(CH₂)ₘ—NR⁷R⁸, hydroxy, phenyl, benzyl, phenylethyl, halogenated phenyl, halogenated benzyl and halogenated phenylethyl, wherein said phenyl, benzyl, phenylethyl, halogenated phenyl, halogenated benzyl and halogenated phenylethyl are optionally substituted with one or more groups selected from —OH, methoxy, ethoxy, halogenated C₁₋₆alkyl, and C₁₋₆alkyl, wherein said R⁷ and R⁸ are independently selected from —H, C₁₋₆alkyl, C₆₋₁₀aryl, C₁₋₅heterocyclyl, and C₃₋₆cycloalkyl-C₀₋₄alkyl, wherein said C₁₋₁₆alkyl, C₆₋₁₀aryl, C₁₋₁₅heterocyclyl, and C₃₋₆cycloalkyl-C₀₋₄alkyl used in defining R⁷ and R⁸ are optionally substituted with one or more groups selected from —OH, methoxy, ethoxy and halogen.

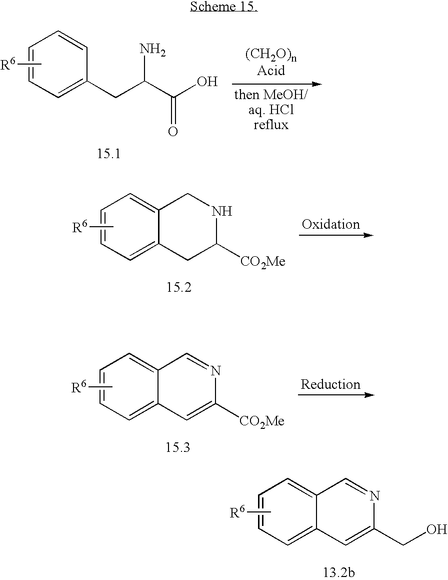

The synthesis of amines of Formula 1.3a, where R³ and R⁴ come together to form a piperazine substituted with an acyl group, can be performed starting from a Boc-pyrazines 16.1 and treating with acylating agents, such as anhydrides and acyl chlorides, optionally in the presence of a mild base, such as Et₃N or DIPEA, in a suitable solvent such as dichloromethane leading to acylated derivatives 16.3. Alternatively, Boc-piperazines 16.1 can be reacted with carboxylic acids 16.2 under standard peptide coupling conditions, such as HOBT/DCC, HOAT/HATU, HOBT/HBTU, EDCI, in the presence of a base such as DIPEA or Et₃N in a suitable solvent such as THF or dichloromethane. Boc-piperazine 16.1, where R¹¹ is 3-oxo, can also be treated with a base such as sodium hydride, and then exposed to an alkyl halide or alkyl sulfonate of Formula 16.4 in a suitable solvent such as ether or THF. The resulting alkyl carbamate 16.5 can be treated with an acid, such as HCl or TFA, in a suitable solvent such as dichloromethane, 1,4-dioxane or THF to provide compounds of Formula 1.3b where NR³R⁴ is an optionally substituted alkylpiperazinone. R¹¹ of Scheme 16 may be selected from hydrogen, oxo, C₁₋₆alkyl, halogenated C₁₋₆alkyl, halogen, methoxy, ethoxy, and morpholinyl.

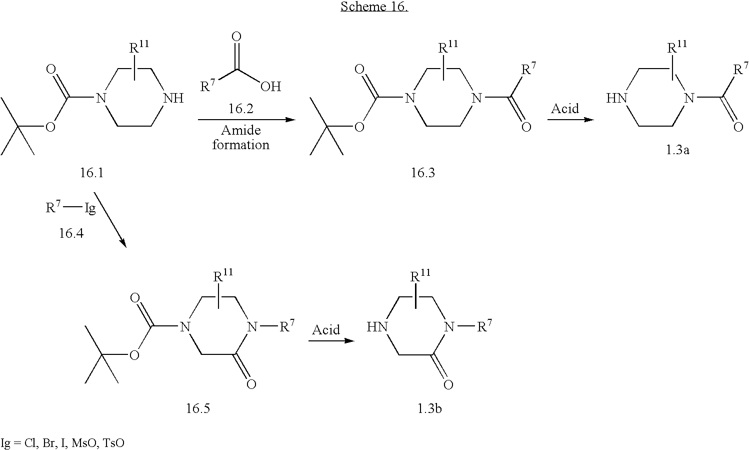

### General Procedures

### General Procedure 1 for Preparation of 5,6-dihydro-7H-pyrrolo[3,4-d]pyrimidin-7-ones

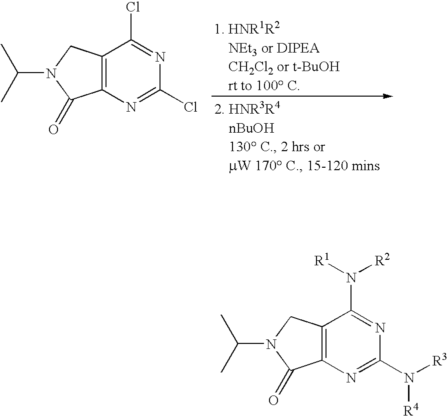

To a solution of 2,4-dichloro-6-isopropyl-5,6-dihydropyrrolo[3,4-d]pyrimidine-7-one (Intermediate 3, 1.0 equiv.) and amine HNR¹R² (1.0 equiv.) in dichloromethane (9.85 mL/mmol pyrrolopyrimidine) or t-butanol (9.85 mL/mmol pyrrolopyrimidine) is added either triethylamine (2.0 equiv.) or diisopropylethylamine (2.0 equiv.). The reaction is stirred for 16 h at rt, or heated up to 100° C., and then concentrated under reduced pressure. The residue is dissolved in n-butanol (9.85 mL/mmol pyrrolopyrimidine) and amine HNR³R⁴ (2.0 equiv.) is added. The reaction is heated in a microwave reactor at 170° C. for 15 to 120 minutes or at 130° C. for 1 to 2 h with conventional heating. The reaction mixture is then cooled to rt and concentrated under reduced pressure. The residue is purified by silica gel chromatography followed by preparative HPLC, or directly purified by preparative HPLC to provide the title compound.

### General Procedure 2 for Preparation of 4-[(2-phenyl-1,1-dimethylethyl)amino]-5,6-dihydro-7H-pyrrolo[3,4-d]pyrimidin-7-ones

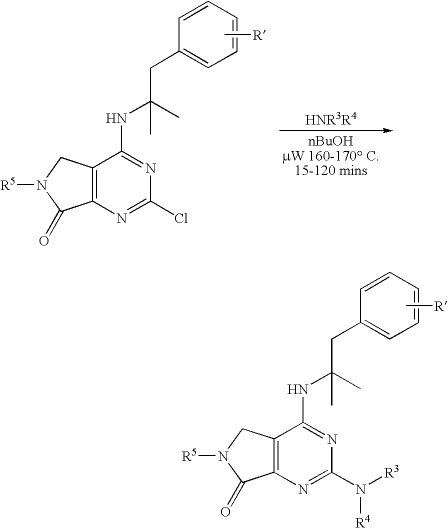

A solution of chloropyrimidine (1.0 equiv.) and HNR³R⁴ (2.0 equiv.) in n-butanol (9.85 mL/mmol pyrrolopyrimidine) is heated in a microwave reactor at 160-170° C. for 15 to 120 minutes. The reaction mixture is then cooled to rt and concentrated under reduced pressure. The residue is purified by silica gel chromatography followed by reverse phase HPLC to provide the title compound.

### General procedure 3 for Preparation of 5,6-dihydro-7H-pyrrolo[3,4-d]pyrimidin-7-ones

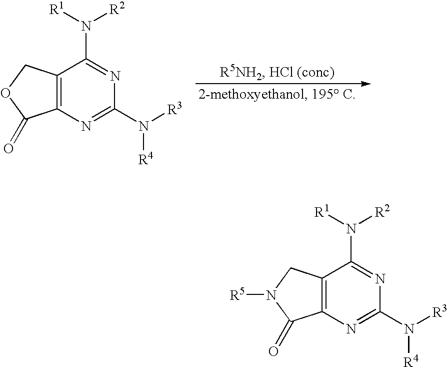

Concentrated HCl (4.4 mmol) is added drop wise to R⁵NH₂ (1.48 mmol) with stirring. The resulting amine hydrochloride is added to a solution of Intermediate 95-98 (0.49 mmol) in 2-methoxyethanol (0.3 mL) in a sealed tube. The reaction mixture is heated in an oil bath at 195° C. for 7-8 h. The reaction mixture is cooled to rt then diluted with CH₂Cl₂ (˜20 mL) and water (˜20 mL). The organic layer is separated, dried over Na₂SO₄, filtered and concentrated under reduced pressure. The product is purified by either silica gel chromatography or preparative HPLC to provide the title compounds.

### General Procedure 4 for Preparation of 5,6-dihydro-7H-pyrrolo[3,4-d]pyrimidin-7-ones

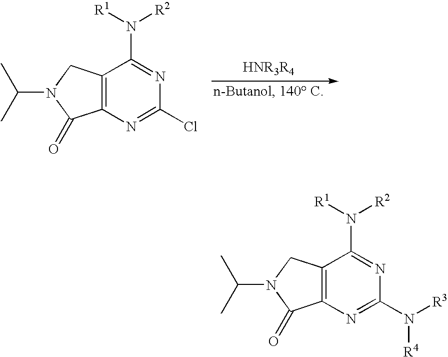

HNR³R⁴ (2.4 mmol) is added to a suspension of 2-chloro-6-isopropyl-5,6-dihydro-7H-pyrrolo[3,4-d]pyrimidin-7-ones (0.3 mmol) in n-BuOH (0.5 mL) in a sealed tube. The reaction mixture is placed in an oil bath preheated to 140° C. and stirred for 18 h. After cooling to rt, the reaction mixture is diluted with CH₂Cl₂ (15 mL) and saturated aqueous NaHCO₃ (15 mL). The organic layer is separated and the aqueous solution is extracted with CH₂Cl₂ (2×15 mL). The organic extracts are combined, dried over MgSO₄, filtered and then concentrated under reduced pressure. The product is purified by silica gel chromatography or recrystallization from organic solvents to provide the corresponding diamino substituted compound.

### General Procedure 5 for Preparation of 6-isopropyl-4-(quinolin-3-ylmethylamino)-5H-pyrrolo[3,4-d]pyrimidin-7(6H)-one

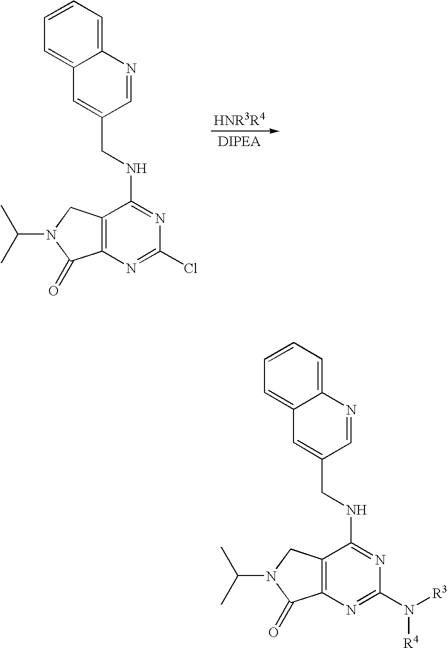

To a solution of 2-chloro-6-isopropyl-4-(quinolin-3-ylmethylamino)-5H-pyrrolo[3,4-d]pyrimidin-7(6H)-one (Intermediate 109) (1.0 equiv.) and amine HNR³R⁴ (1.05-1.5 equiv.) in n-butanol or i-PrOH (5.7 mL/mmol pyrrolopyrimidine) is added DIPEA (1.2-2.0 equiv.). The reaction is heated up to 160° C. in a microwave reactor for 30-60 minutes, cooled to rt and then concentrated under reduced pressure. The residue is purified by preparative LCMS (high pH, X-Bridge Prep C₁₈ OBD, 30×50 mm, 5 μm particle size) to provide the title compounds.

### General Procedure 6 for Preparation of 2-(4-acetylpiperazin-1-yl)-6-isopropyl-5,6-dihydro-7H-pyrrolo[3,4-d]pyrimidin-7-ones

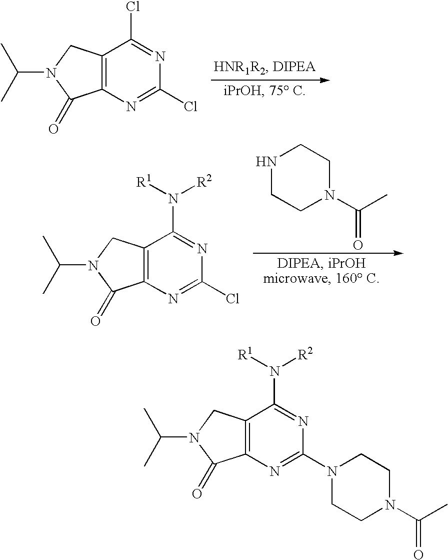

To a solution of 2,4-dichloro-6-isopropyl-5,6-dihydropyrrolo[3,4-d]pyrimidine-7-one (Intermediate 3, 1.0 equiv.) in n-BuOH or 1,2-dichloroethane or i-PrOH (6-9 mL/mmol) is added HNR¹R² (1.05 equiv.) followed by DIPEA (1.0 equiv.). The mixture is stirred at 75° C. for 1 h, cooled to rt and the reaction mixture is transferred to a thick-walled microwave glass vial charged with a stirring bar, then 1-(piperazin-1-yl)ethanone (1.2-2.0 equiv.) is added followed by DIPEA (1.2-2.0 equiv.). The reaction vial is sealed and subjected to microwave radiation at 160° C. for 1 h. The mixture is concentrated under reduced pressure, and the residue is purified with preparative HPLC or preparative LCMS (high pH, X-Bridge Prep C₁₈ OBD, 30×50 mm, 5 μm particle size) to give the title compounds.

### Biological Evaluation

### Biological Evaluation of Compounds as Antagonists at Human P2X3 Receptors and Rat P2X3 In Vitro

The antagonist properties of compounds in the present invention are assayed as inhibition of the intracellular calcium increase induced by activation of hP2X3 (human Purinergic P2X receptors subtype 3, accession number AB016608 for clone A and accession number NM_{—}002559 for clone B), expressed in RLE cells (rat liver endothelium, ATCC) as well as for the rat P2X3 (gene accession number NM_{—}031075.1) expressed in HEK-293s cells (Human Embrionic Kidney cells, ATCC) and for the rat P2X3 co-expressed with the rat P2X2 in HEK-TREX cells (Invitrogene, inducible system). The assay used a calcium indicator dye (Fluo-4) that emits fluorescence, the intensity of which is related to the concentration of calcium that entered the cell when P2X3 was activated and the channel opened. Activation of hP2X3 or rat P2X3 and rat P2X2/3 (from the coexpression of rat P2X3 and rat P2×2) is elicited the by P2X3 agonist α,β methylene-ATP (Sigma M6517), and the resulting fluorescence is measured with a FLIPR II™ instrument (Molecular Devices). Compounds are tested for their ability to inhibit the agonist-induced fluorescent signal. The RLE/hP2X3 cells are grown in William's medium 1× (Gibco, 12551-032) supplemented with 10% Foetal bovine serum (Wisent, 090850), 2 mM L-Glutamine (Wisent, 609-065-EL), and 600 μg/mL Geneticin G-418 (Wisent, 61234) in a humidified incubator (5% CO₂ and 37° C.). The rat P2X3 and the rat P2X2/3 cells line are grown in DMEM medium 1× (Wisent, 319 005 CL) supplemented with 10% Foetal bovine serum (Wisent, 090850), 2 mM L-Glutamine (Wisent, 609-065-EL), and 600 μg/mL Geneticin G-418 (Wisent, 61234) in a humidified incubator (5% CO₂ and 37° C.).

The day before the experiment, hP2X3 cells are plated in 384-black polylysine coated plates (Becton/Dickinson, 356663) at 8000 cells/well in 50 μL/well in William's medium without Geneticin, and placed in the incubator for 24 h. For the rat P2X2/3 HEK-TREX cells, induction of the rat P2X3 expression is used to generate the rat P2X2/3 channels in HEK-TREX cells and performed by addition of 1 μg/mL tetracycline (Invitrogen) 24 h prior to compounds testing to the HEK-TREX expressing the rat P2X2 constitutively. On the day of the experiment, the cells and test compounds are prepared as follows. For the compounds, α,β-methylene-ATP (500 nM, final concentration) and reference compounds (spanning a range of 10 dilutions, three-fold apart) are diluted, at a concentration 4-fold higher than the desired final concentration, into the hP2X3 assay buffer (125 mM Choline chloride, 5 mM Glucose, 0.2 g/L BSA, 25 mM Hepes, 5 mM KCl, 1 mM MgCl₂, 1.5 mM CaCl₂, pH 7.4) or alternatively in the rat P2X3 & rat P2X2/3 assay buffer (HBSS: 125 mM NaCl, 5 mM Glucose, 0.2 g/L BSA, 25 mM Hepes, 5 mM KCl, 1 mM MgCl₂, 1.5 mM CaCl₂, pH 7.4). After preparing the compounds, the medium is removed from the cell plates by inversion. A loading solution of 30 μL assay buffer containing 4 μM of the calcium indicator dye FLUO-4 AM (Molecular Probes F14202) is added to each well using a Multidrop (Labsystems). The cell plates are then incubated at rt for 30-40 minutes to allow loading of the dye into the cells. The incubation is terminated by washing the cells four times in assay buffer using a Skatron Embla (Molecular Devices), and 25 μL of assay buffer was left in each well. Cell plates are then transferred to the FLIPR. Experiments are initiated by measuring a baseline fluorescence reading for 10 seconds, followed by the addition of 12.5 μL of cpds and data sampling for a total 280 seconds. The experiments are terminated by addition of 12.5 μL of a reference agonist (500 nM α,β-methylene-ATP) or buffer, producing a final assay volume of 50 μL followed by data sampling for an additional 280 seconds. During entire experiment, fluorescence emission is read by the FLIPR on board CCD camera using filter with emission wavelength of 520-545 nm.

In P2X3 antagonist experiments (human and rat assays) data are analysed as normalised maximal peak fluorescence monitored following agonist addition and calculated as percent of relative fluorescent counts from control agonist. The dose-response antagonist inhibition curves are analyzed in a 4-parameter sigmoidal fit using a non-linear curve-fitting program (XLfit version 5.0.6, ID Business Solutions Limited, Guildford, UK). The fitted top (extrapolated zero effect) maximum inhibition (Emax), Hill slope and IC₅₀ are calculated for each compound and the latter three values are used for establishing structure activity relationship of compounds cited in the present invention.

The following table shows IC₅₀ (nM) for human P2X3 and rat P2X2/3 receptors for some of the exemplified compounds when measured using the assays described above.

| | | Human | Human | |
|---|---|---|---|---|
| | | P2X3 | P2X3 | Rat |
| | | FLIPR | FLIPR | P2X2/3 |
| | | Clone A | Clone B | FLIPR |
| Compound | | Mean | IC50 | Mean |
| Name | Compound # | IC50 (nM) | (nM) | IC50 (nM) |
| 2-(4-acetylpiperazin-1-yl)-6-isopropyl-4- | 1 | | 4800 | |
| (1,2,3,4-tetrahydronaphthalen-1-ylamino)- | | | | |
| 5,6-dihydro-7H-pyrrolo[3,4-d]pyrimidin-7- | | | | |
| one | | | | |
| ethyl 3-{[2-(4-acetylpiperazin-1-yl)-6- | 2 | | 1900 | |
| isopropyl-7-oxo-6,7-dihydro-5H- | | | | |
| pyrrolo[3,4-d]pyrimidin-4-yl]amino}-2- | | | | |
| phenylpropanoate | | | | |
| 2-(4-acetylpiperazin-1-yl)-4- | 3 | | 4600 | |
| [benzyl(tetrahydrofuran-2- | | | | |
| ylmethyl)amino]-6-isopropyl-5,6-dihydro- | | | | |
| 7H-pyrrolo[3,4-d]pyrimidin-7-one | | | | |
| 2-(4-acetylpiperazin-1-yl)-4- | 4 | | 800 | |
| [cyclopentyl(4-fluorobenzyl)amino]-6- | | | | |
| isopropyl-5,6-dihydro-7H-pyrrolo[3,4- | | | | |
| d]pyrimidin-7-one | | | | |
| 2-(4-acetylpiperazin-1-yl)-4-{[1-(4-tert- | 5 | | 440 | |
| butylphenyl)ethyl]amino}-6-isopropyl-5,6- | | | | |
| dihydro-7H-pyrrolo[3,4-d]pyrimidin-7-one | | | | |
| 2-(4-acetylpiperazin-1-yl)-4-{[1-(4- | 6 | | 160 | |
| isobutylphenyl)ethyl]amino}-6-isopropyl- | | | | |
| 5,6-dihydro-7H-pyrrolo[3,4-d]pyrimidin-7- | | | | |
| one | | | | |
| 2-{[2-(dimethylamino)ethyl]amino}-6- | 7 | | 85 | |
| isopropyl-4-{[(4- | | | | |
| methylphenyl)(phenyl)methyl]amino}-5,6- | | | | |
| dihydro-7H-pyrrolo[3,4-d]pyrimidin-7-one | | | | |
| 2-(4-acetylpiperazin-1-yl)-4-{[2-(4- | 8 | | 440 | |
| chlorophenyl)propyl]amino}-6-isopropyl- | | | | |
| 5,6-dihydro-7H-pyrrolo[3,4-d]pyrimidin-7- | | | | |
| one | | | | |
| 4-{[(4- | 9 | | 91 | |
| chlorophenyl)(phenyl)methyl]amino}-2- | | | | |
| {[2-(dimethylamino)ethyl]amino}-6- | | | | |
| isopropyl-5,6-dihydro-7H-pyrrolo[3,4- | | | | |
| d]pyrimidin-7-one | | | | |
| 2-(4-acetylpiperazin-1-yl)-6-isopropyl-4- | 10 | | 1200 | |
| {[1-(4-isopropylphenyl)-2- | | | | |
| methylpropyl]amino}-5,6-dihydro-7H- | | | | |
| pyrrolo[3,4-d]pyrimidin-7-one | | | | |
| 2-(4-acetylpiperazin-1-yl)-6-isopropyl-4- | 11 | | 2500 | |
| {[(1R)-1-(4-methoxyphenyl)ethyl]amino}- | | | | |
| 5,6-dihydro-7H-pyrrolo[3,4-d]pyrimidin-7- | | | | |
| one | | | | |
| 2-(4-acetylpiperazin-1-yl)-4-{[2-(4- | 12 | | 3900 | |
| fluorophenyl)-1-methylethyl]amino}-6- | | | | |
| isopropyl-5,6-dihydro-7H-pyrrolo[3,4- | | | | |
| d]pyrimidin-7-one | | | | |
| 2-(4-acetylpiperazin-1-yl)-6-isopropyl-4- | 13 | | 220 | |
| {[1-(3-phenyl-1,2,4-oxadiazol-5- | | | | |
| yl)ethyl]amino}-5,6-dihydro-7H- | | | | |
| pyrrolo[3,4-d]pyrimidin-7-one | | | | |
| 2-(4-acetylpiperazin-1-yl)-4-{[2-(4- | 14 | | 110 | 18470 |
| fluorophenyl)-1,1-dimethylethyl]amino}-6- | | | | |
| isopropyl-5,6-dihydro-7H-pyrrolo[3,4- | | | | |
| d]pyrimidin-7-one | | | | |
| 2-(4-acetylpiperazin-1-yl)-4-({[1-(4- | 15 | | 260 | |
| chlorophenyl)cyclobutyl]methyl}amino)-6- | | | | |
| isopropyl-5,6-dihydro-7H-pyrrolo[3,4- | | | | |
| d]pyrimidin-7-one | | | | |
| 2-(4-acetylpiperazin-1-yl)-4-{[2-(4- | 16 | | 2100 | |
| chlorophenyl)-2-methylpropyl]amino}-6- | | | | |
| isopropyl-5,6-dihydro-7H-pyrrolo[3,4- | | | | |
| d]pyrimidin-7-one | | | | |
| 4-{[bis(4-fluorophenyl)methyl]amino}-2- | 17 | | 70 | |
| {[2-(dimethylamino)ethyl]amino}-6- | | | | |
| isopropyl-5,6-dihydro-7H-pyrrolo[3,4- | | | | |
| d]pyrimidin-7-one | | | | |
| 4-{[(4- | 18 | | 180 | |
| chlorophenyl)(phenyl)methyl]amino}-2-(4- | | | | |
| ethylpiperazin-1-yl)-6-isopropyl-5,6- | | | | |
| dihydro-7H-pyrrolo[3,4-d]pyrimidin-7-one | | | | |
| 4-{[(4- | 19 | | 93 | |
| chlorophenyl)(phenyl)methyl]amino}-2- | | | | |
| [[2-(dimethylamino)ethyl](methyl)amino]- | | | | |
| 6-isopropyl-5,6-dihydro-7H-pyrrolo[3,4- | | | | |
| d]pyrimidin-7-one | | | | |
| 4-{[(4- | 20 | | 100 | |
| chlorophenyl)(phenyl)methyl]amino}-6- | | | | |
| isopropyl-2-(5-methyl-2,5- | | | | |
| diazabicyclo[2.2.1]hept-2-yl)-5,6-dihydro- | | | | |
| 7H-pyrrolo[3,4-d]pyrimidin-7-one | | | | |
| 2-{[2-(dimethylamino)ethyl]amino}-4-[(9- | 21 | | 140 | |
| fluoro-10,11-dihydro-5H- | | | | |
| benzo[4,5]cyclohepta[1,2-b]pyridin-5- | | | | |
| yl)amino]-6-isopropyl-5,6-dihydro-7H- | | | | |
| pyrrolo[3,4-d]pyrimidin-7-one | | | | |
| 2-{[2-(dimethylamino)ethyl]amino}-4-[(7- | 22 | | 270 | |
| fluoro-10,11-dihydro-5H- | | | | |
| benzo[4,5]cyclohepta[1,2-b]pyridin-5- | | | | |
| yl)amino]-6-isopropyl-5,6-dihydro-7H- | | | | |
| pyrrolo[3,4-d]pyrimidin-7-one | | | | |
| 4-{[bis(4-fluorophenyl)methyl]amino}-6- | 23 | | 210 | |
| isopropyl-2-(5-methyl-2,5- | | | | |
| diazabicyclo[2.2.1]hept-2-yl)-5,6-dihydro- | | | | |
| 7H-pyrrolo[3,4-d]pyrimidin-7-one | | | | |
| 4-{[bis(4-fluorophenyl)methyl]amino}-2- | 24 | | 190 | |
| [[2-(dimethylamino)ethyl](methyl)amino]- | | | | |
| 6-isopropyl-5,6-dihydro-7H-pyrrolo[3,4- | | | | |
| d]pyrimidin-7-one | | | | |
| 2-(4-acetylpiperazin-1-yl)-6-isopropyl-4- | 25 | | 39 | |
| ({1-[4- | | | | |
| (trifluoromethoxy)phenyl]ethyl}amino)- | | | | |
| 5,6-dihydro-7H-pyrrolo[3,4-d]pyrimidin-7- | | | | |
| one | | | | |
| 2-(4-acetylpiperazin-1-yl)-4-{[1-(4- | 26 | | 110 | 2897 |
| ethoxyphenyl)ethyl]amino}-6-isopropyl- | | | | |
| 5,6-dihydro-7H-pyrrolo[3,4-d]pyrimidin-7- | | | | |
| one | | | | |
| 2-(4-acetylpiperazin-1-yl)-4-{[(4- | 27 | | 140 | |
| chlorophenyl)(cyclopropyl)methyl]amino}- | | | | |
| 6-isopropyl-5,6-dihydro-7H-pyrrolo[3,4- | | | | |
| d]pyrimidin-7-one | | | | |
| 2-(4-acetylpiperazin-1-yl)-6-isopropyl-4- | 28 | | 200 | |
| ({1-[4- | | | | |
| (trifluoromethyl)phenyl]ethyl}amino)-5,6- | | | | |
| dihydro-7H-pyrrolo[3,4-d]pyrimidin-7-one | | | | |
| 2-(4-acetylpiperazin-1-yl)-6-isopropyl-4- | 29 | | 160 | |
| {[1-(4-propylphenyl)ethyl]amino}-5,6- | | | | |
| dihydro-7H-pyrrolo[3,4-d]pyrimidin-7-one | | | | |
| 2-(4-acetylpiperazin-1-yl)-6-isopropyl-4- | 30 | | 160 | |
| {[4-(trifluoromethoxy)benzyl]amino}-5,6- | | | | |
| dihydro-7H-pyrrolo[3,4-d]pyrimidin-7-one | | | | |
| 5-[2-(4-{4-[(4-chlorophenyl)amino]-6- | 31 | | 270 | |
| isopropyl-7-oxo-6,7-dihydro-5H- | | | | |
| pyrrolo[3,4-d]pyrimidin-2-yl}piperazin-1- | | | | |
| yl)-2-oxoethyl]imidazolidine-2,4-dione | | | | |
| 2-(4-acetylpiperazin-1-yl)-6-isopropyl-4- | 32 | | 430 | |
| ({1-[4- | | | | |
| (trifluoromethyl)phenyl]propyl}amino)- | | | | |
| 5,6-dihydro-7H-pyrrolo[3,4-d]pyrimidin-7- | | | | |
| one | | | | |
| 2-(4-acetylpiperazin-1-yl)-4-{[trans-2-(4- | 33 | | 580 | |
| chlorophenyl)cyclopentyl]amino}-6- | | | | |
| isopropyl-5,6-dihydro-7H-pyrrolo[3,4- | | | | |
| d]pyrimidin-7-one | | | | |
| 2-(4-acetylpiperazin-1-yl)-6-isopropyl-4- | 34 | | 610 | |
| {[(1R)-1-(4-methylphenyl)ethyl]amino}- | | | | |
| 5,6-dihydro-7H-pyrrolo[3,4-d]pyrimidin-7- | | | | |
| one | | | | |
| {[2-(4-acetylpiperazin-1-yl)-6-isopropyl-7- | 35 | | 1200 | |
| oxo-6,7-dihydro-5H-pyrrolo[3,4- | | | | |
| d]pyrimidin-4-yl]amino}[4- | | | | |
| (trifluoromethyl)phenyl]acetic acid | | | | |
| 2-(4-acetylpiperazin-1-yl)-6-isopropyl-4- | 36 | | 1800 | |
| {[1-(4-methylphenyl)propyl]amino}-5,6- | | | | |
| dihydro-7H-pyrrolo[3,4-d]pyrimidin-7-one | | | | |
| 4-[(4-benzylphenyl)amino]-6-isopropyl-2- | 37 | | >30000 | |
| morpholin-4-yl-5,6-dihydro-7H- | | | | |
| pyrrolo[3,4-d]pyrimidin-7-on | | | | |
| 2-(4-acetylpiperazin-1-yl)-6-isopropyl-4- | 38 | | 12000 | |
| {[(1-methyl-1,2,3,4-tetrahydroquinolin-6- | | | | |
| yl)methyl]amino}-5,6-dihydro-7H- | | | | |
| pyrrolo[3,4-d]pyrimidin-7-one | | | | |
| 2-(4-acetylpiperazin-1-yl)-4-[2-ethyl-2-(4- | 39 | | 10000 | |
| methylphenyl)hydrazino]-6-isopropyl-5,6- | | | | |
| dihydro-7H-pyrrolo[3,4-d]pyrimidin-7-one | | | | |
| 2-(4-acetylpiperazin-1-yl)-6-isopropyl-4- | 40 | | 310 | |
| {[1-(4-isopropylphenyl)ethyl]amino}-5,6- | | | | |
| dihydro-7H-pyrrolo[3,4-d]pyrimidin-7-one | | | | |
| 2-(4-acetylpiperazin-1-yl)-4-{[2-(4- | 41 | | 1700 | |
| chlorophenyl)ethyl]amino}-6-isopropyl- | | | | |
| 5,6-dihydro-7H-pyrrolo[3,4-d]pyrimidin-7- | | | | |
| one | | | | |
| 2-(4-acetylpiperazin-1-yl)-6-isopropyl-4- | 42 | | 1300 | |
| {[2-(4-methylphenyl)ethyl]amino}-5,6- | | | | |
| dihydro-7H-pyrrolo[3,4-d]pyrimidin-7-one | | | | |
| 2-(4-acetylpiperazin-1-yl)-6-isopropyl-4- | 43 | | 650 | |
| [(4-isopropylbenzyl)amino]-5,6-dihydro- | | | | |
| 7H-pyrrolo[3,4-d]pyrimidin-7-one | | | | |
| 4-[2-(4-chlorobenzyl)pyrrolidin-1-yl]-6- | 44 | | 9600 | |
| isopropyl-2-[(2-methoxyethyl)amino]-5,6- | | | | |
| dihydro-7H-pyrrolo[3,4-d]pyrimidin-7-one | | | | |
| N-[2-({4-[2-(4-chlorobenzyl)pyrrolidin-1- | 45 | | 2200 | |
| yl]-6-isopropyl-7-oxo-6,7-dihydro-5H- | | | | |
| pyrrolo[3,4-d]pyrimidin-2- | | | | |
| yl}amino)ethyl]acetamide | | | | |
| 2-(4-acetylpiperazin-1-yl)-4-[2-(2- | 46 | | 2300 | |
| chlorobenzyl)pyrrolidin-1-yl]-6-isopropyl- | | | | |
| 5,6-dihydro-7H-pyrrolo[3,4-d]pyrimidin-7- | | | | |
| one | | | | |
| 2-(4-acetylpiperazin-1-yl)-6-isopropyl-4- | 47 | | 130 | 2682 |
| [2-(4-methylbenzyl)pyrrolidin-1-yl]-5,6- | | | | |
| dihydro-7H-pyrrolo[3,4-d]pyrimidin-7-one | | | | |
| 2-(4-acetylpiperazin-1-yl)-4-[2-(3- | 48 | | 300 | |
| chlorobenzyl)pyrrolidin-1-yl]-6-isopropyl- | | | | |
| 5,6-dihydro-7H-pyrrolo[3,4-d]pyrimidin-7- | | | | |
| one | | | | |
| 4-[2-(4-chlorobenzyl)pyrrolidin-1-yl]-6- | 49 | | 2000 | |
| isopropyl-2-(3-oxopiperazin-1-yl)-5,6- | | | | |
| dihydro-7H-pyrrolo[3,4-d]pyrimidin-7-one | | | | |
| 2-(4-acetylpiperazin-1-yl)-4-[2-(4- | 50 | | 150 | |
| chlorobenzyl)pyrrolidin-1-yl]-6-isopropyl- | | | | |
| 5,6-dihydro-7H-pyrrolo[3,4-d]pyrimidin-7- | | | | |
| one | | | | |
| 2-(4-acetylpiperazin-1-yl)-4- | 51 | | 2700 | |
| [benzyl(cyclopropylmethyl)amino]-6- | | | | |
| isopropyl-5,6-dihydro-7H-pyrrolo[3,4- | | | | |
| d]pyrimidin-7-one | | | | |
| 2-(4-acetylpiperazin-1-yl)-4-[2-(4- | 52 | | 720 | |
| chlorophenyl)pyrrolidin-1-yl]-6-isopropyl- | | | | |
| 5,6-dihydro-7H-pyrrolo[3,4-d]pyrimidin-7- | | | | |
| one | | | | |
| 2-(4-acetylpiperazin-1-yl)-4-{[1-(4- | 53 | | 750 | |
| chlorophenyl)ethyl]amino}-6-isopropyl- | | | | |
| 5,6-dihydro-7H-pyrrolo[3,4-d]pyrimidin-7- | | | | |
| one | | | | |
| 2-(4-acetylpiperazin-1-yl)-4-(2- | 54 | | 240 | |
| benzylpyrrolidin-1-yl)-6-isopropyl-5,6- | | | | |
| dihydro-7H-pyrrolo[3,4-d]pyrimidin-7-one | | | | |
| 2-(4-acetylpiperazin-1-yl)-4-{[2-(3,4- | 55 | | 9700 | |
| dimethylphenyl)ethyl]amino}-6-isopropyl- | | | | |
| 5,6-dihydro-7H-pyrrolo[3,4-d]pyrimidin-7- | | | | |
| one | | | | |
| 2-(4-acetylpiperazin-1-yl)-4-{[2-(2,4- | 56 | | 2700 | |
| dimethylphenyl)ethyl]amino}-6-isopropyl- | | | | |
| 5,6-dihydro-7H-pyrrolo[3,4-d]pyrimidin-7- | | | | |
| one | | | | |
| 4-[2-(4-chlorobenzyl)pyrrolidin-1-yl]-2- | 57 | | 8500 | |
| {[2-(dimethylamino)ethyl]amino}-6- | | | | |
| isopropyl-5,6-dihydro-7H-pyrrolo[3,4- | | | | |
| d]pyrimidin-7-one | | | | |
| 2-(4-acetylpiperazin-1-yl)-4-[benzyl(4- | 58 | | 270 | |
| chlorobenzyl)amino]-6-isopropyl-5,6- | | | | |
| dihydro-7H-pyrrolo[3,4-d]pyrimidin-7-one | | | | |
| 2-(4-acetylpiperazin-1-yl)-4-[(4- | 59 | | 220 | |
| chlorobenzyl)(cyclopropylmethyl)amino]- | | | | |
| 6-isopropyl-5,6-dihydro-7H-pyrrolo[3,4- | | | | |
| d]pyrimidin-7-one | | | | |
| 2-(4-acetylpiperazin-1-yl)-4-{[2-(4- | 60 | 68.7 | 61 | 1932 |
| chlorophenyl)-1,1-dimethylethyl]amino}- | | | | |
| 6-isopropyl-5,6-dihydro-7H-pyrrolo[3,4- | | | | |
| d]pyrimidin-7-one | | | | |
| 4-{[2-(4-chlorophenyl)-1,1- | 61 | | 1400 | |
| dimethylethyl]amino}-2-[(2R,6S)-2,6- | | | | |
| dimethylmorpholin-4-yl]-6-isopropyl-5,6- | | | | |
| dihydro-7H-pyrrolo[3,4-d]pyrimidin-7-one | | | | |
| N-[1-(4-{[2-(4-chlorophenyl)-1,1- | 62 | | 340 | |
| dimethylethyl]amino}-6-isopropyl-7-oxo- | | | | |
| 6,7-dihydro-5H-pyrrolo[3,4-d]pyrimidin-2- | | | | |
| yl)pyrrolidin-3-yl]acetamide | | | | |
| 2-[4-(4-{[2-(4-chlorophenyl)-1,1- | 63 | | 9200 | |
| dimethylethyl]amino}-6-isopropyl-7-oxo- | | | | |
| 6,7-dihydro-5H-pyrrolo[3,4-d]pyrimidin-2- | | | | |
| yl)piperazin-1-yl]-N,N-dimethylacetamide | | | | |
| 4-{[2-(4-chlorophenyl)-1,1- | 64 | | 3500 | |
| dimethylethyl]amino}-2-(4-ethylpiperazin- | | | | |
| 1-yl)-6-isopropyl-5,6-dihydro-7H- | | | | |
| pyrrolo[3,4-d]pyrimidin-7-one | | | | |
| 2-(4-acetyl-1,4-diazepan-1-yl)-4-{[2-(4- | 65 | | 420 | |
| chlorophenyl)-1,1-dimethylethyl]amino}- | | | | |
| 6-isopropyl-5,6-dihydro-7H-pyrrolo[3,4- | | | | |
| d]pyrimidin-7-one | | | | |
| 4-{[2-(4-chlorophenyl)-1,1- | 66 | | 1400 | |
| dimethylethyl]amino}-6-isopropyl-2-{[3- | | | | |
| (2-oxopyrrolidin-1-yl)propyl]amino}-5,6- | | | | |
| dihydro-7H-pyrrolo[3,4-d]pyrimidin-7-one | | | | |
| N-[1-(4-{[2-(4-chlorophenyl)-1,1- | 67 | | 450 | |
| dimethylethyl]amino}-6-isopropyl-7-oxo- | | | | |
| 6,7-dihydro-5H-pyrrolo[3,4-d]pyrimidin-2- | | | | |
| yl)pyrrolidin-3-yl]-N-methylacetamide | | | | |
| 4-{[2-(4-chlorophenyl)-1,1- | 68 | | 350 | |
| dimethylethyl]amino}-6-isopropyl-2-(5- | | | | |
| oxo-1,4-diazepan-1-yl)-5,6-dihydro-7H- | | | | |
| pyrrolo[3,4-d]pyrimidin-7-one | | | | |
| 4-{[2-(4-chlorophenyl)-1,1- | 69 | 86.19 | 140 | 1923 |
| dimethylethyl]amino}-6-isopropyl-2-(4- | | | | |
| methyl-3-oxopiperazin-1-yl)-5,6-dihydro- | | | | |
| 7H-pyrrolo[3,4-d]pyrimidin-7-one | | | | |
| 4-{[2-(4-chlorophenyl)-1,1- | 70 | | 780 | |
| dimethylethyl]amino}-6-isopropyl-2-{[2- | | | | |
| (2-oxopyrrolidin-1-yl)ethyl]amino}-5,6- | | | | |
| dihydro-7H-pyrrolo[3,4-d]pyrimidin-7-one | | | | |
| 4-{[2-(4-chlorophenyl)-1,1- | 71 | | 130 | |
| dimethylethyl]amino}-6-isopropyl-2-(4- | | | | |
| propionylpiperazin-1-yl)-5,6-dihydro-7H- | | | | |
| pyrrolo[3,4-d]pyrimidin-7-one | | | | |
| 2-(4-Acetyl-piperazin-1-yl)-4-(benzhydryl- | 72 | | 270 | 2110 |
| amino)-6-isopropyl-5,6-dihydro- | | | | |
| pyrrolo[3,4-d]pyrimidin-7-one | | | | |
| 4-(Benzhydryl-amino)-6-isobutyl-2- | 73 | | 3800 | |
| morpholin-4-yl-5,6-dihydro-pyrrolo[3,4- | | | | |
| d]pyrimidin-7-one | | | | |
| 4-(Benzhydryl-amino)-6-cyclopropyl-2- | 74 | | 4300 | |
| morpholin-4-yl-5,6-dihydro-pyrrolo[3,4- | | | | |
| d]pyrimidin-7-one | | | | |
| 4-(Benzhydryl-amino)-6-cyclopentyl-2- | 75 | | >30000 | |
| morpholin-4-yl-5,6-dihydro-pyrrolo[3,4- | | | | |
| d]pyrimidin-7-one | | | | |
| 6-isopropyl-2-morpholin-4-yl-[(phenyl-p- | 76 | | 230 | |
| tolyl-methyl)-amino]-5,6-dihydro- | | | | |
| pyrrolo[3,4-d]pyrimidin-7-one | | | | |
| 4-{[(4-Chloro-phenyl)-phenyl-methyl]- | 77 | | 260 | |
| amino}-6-isopropyl-2-morpholin-4-yl-5,6- | | | | |
| dihydro-pyrrolo[3,4-d]pyrimidin-7-one | | | | |
| 4-(Benzhydryl-amino)-2-(4-ethyl- | 78 | | 400 | |
| piperazin-1-yl)-6-isopropyl-5,6-dihydro- | | | | |
| pyrrolo[3,4-d]pyrimidin-7-one | | | | |
| 4-(Benzhydryl-amino)-2-(4- | 79 | | 130 | |
| cyclopropanecarbonyl-piperazin-1-yl)-6- | | | | |
| isopropyl-5,6-dihydro-pyrrolo[3,4- | | | | |
| d]pyrimidin-7-one | | | | |
| 4-(Benzhydryl-amino)-2-(4-isobutyryl- | 80 | | 190 | |
| piperazin-1-yl)-6-isopropyl-5,6-dihydro- | | | | |
| pyrrolo[3,4-d]pyrimidin-7-one | | | | |
| 4-(Benzhydryl-amino)-6-isopropyl-2- | 81 | | 1400 | |
| morpholin-4-yl-5,6-dihydro-pyrrolo[3,4- | | | | |
| d]pyrimidin-7-one | | | | |
| 4-(Benzhydryl-amino)-6-dimethylamino-2- | 82 | | 1900 | |
| (4-methyl-piperazin-1-yl)-5,6-dihydro- | | | | |
| cyclopentapyrimidin-7-one | | | | |
| 4-(Benzhydryl-amino)-6-isopropyl-2- | 83 | | 620 | |
| piperazin-1-yl-5,6-dihydro-pyrrolo[3,4- | | | | |
| d]pyrimidin-7-one | | | | |
| 4-(Benzhydryl-amino)-2-benzylamino-6- | 84 | | 830 | |
| isopropyl-5,6-dihydro-pyrrolo[3,4- | | | | |
| d]pyrimidin-7-one | | | | |
| 4-(Benzhydryl-amino)-6-isopropyl-2-(2- | 85 | | 250 | |
| methoxy-ethylamino)-5,6-dihydro- | | | | |
| pyrrolo[3,4-d]pyrimidin-7-one | | | | |
| 4-(Benzhydryl-amino)-2-(2,6-dimethyl- | 86 | | 280 | |
| morpholin-4-yl)-6-isopropyl-5,6-dihydro- | | | | |
| pyrrolo[3,4-d]pyrimidin-7-one | | | | |
| 4-(Benzhydryl-amino)-6-isopropyl-2-(4- | 87 | | 240 | |
| propionyl-piperazin-1-yl)-5,6-dihydro- | | | | |
| pyrrolo[3,4-d]pyrimidin-7-one | | | | |
| 2-(4-Acetyl-piperazin-1-yl)-4-(1,2- | 88 | | 1800 | |
| diphenyl-ethylamino)-6-isopropyl-5,6- | | | | |
| dihydro-pyrrolo[3,4-d]pyrimidin-7-one | | | | |
| 4-(Benzhydryl-amino)-2-diethylamino-6- | 89 | | 6200 | |
| isopropyl-5,6-dihydro-pyrrolo[3,4- | | | | |
| d]pyrimidin-7-one | | | | |
| 2-{4-[4-(Benzhydryl-amino)-6-isopropyl- | 90 | | 440 | |
| 7-oxo-6,7-dihydro-5H-pyrrolo[3,4- | | | | |
| d]pyrimidin-2-yl]-piperazin-1-yl}-N,N- | | | | |
| dimethyl-acetamide | | | | |
| 2-(4-acetylpiperazin-1-yl)-4-[(2,2- | 91 | | 100 | |
| diphenylethyl)amino]-6-isopropyl-5,6- | | | | |
| dihydro-7H-pyrrolo[3,4-d]pyrimidin-7-one | | | | |
| 4-(Benzhydryl-amino)-6-isopropyl-2-(4- | 92 | | 400 | |
| methanesulfonyl-piperazin-1-yl)-5,6- | | | | |
| dihydro-pyrrolo[3,4-d]pyrimidin-7-one | | | | |
| N-{2-[4-(Benzhydryl-amino)-6-isopropyl- | 93 | | 290 | |
| 7-oxo-6,7-dihydro-5H-pyrrolo[3,4- | | | | |
| d]pyrimidin-2-ylamino]-ethyl}-acetamide | | | | |
| 4-(Benzhydryl-amino)-6-isopropyl-2- | 94 | | 250 | |
| [(pyridin-3-ylmethyl)-amino]-5,6-dihydro- | | | | |
| pyrrolo[3,4-d]pyrimidin-7-one | | | | |
| 2-(4-Acetyl-piperazin-1-yl)-4- | 95 | | 430 | |
| dibenzylamino-6-isopropyl-5,6-dihydro- | | | | |
| pyrrolo[3,4-d]-pyrimidin-7-one | | | | |
| N-{1-[4-(Benzhydryl-amino)-6-isopropyl- | 96 | | 330 | |
| 7-oxo-6,7-dihydro-5H-pyrrolo[3,4- | | | | |
| d]pyrimidin-2-yl]-pyrrolidin-3-yl}- | | | | |
| acetamide | | | | |
| 4-(Benzhydryl-amino)-2-(2- | 97 | | 230 | 10000 |
| dimethylamino-ethylamino)-6-isopropyl- | | | | |
| 5,6-dihydro-pyrrolo[3,4-d]pyrimidin-7-one | | | | |
| 6-isopropyl-2-(2-methoxy-ethylamino)-4- | 98 | | 210 | |
| [(phenyl-p-tolyl-methyl)-amino]-5,6- | | | | |
| dihydro-pyrrolo[3,4-d]-pyrimidin-7-one | | | | |
| 4-(Benzhydryl-amino)-2-[4-(2- | 99 | | 5700 | |
| dimethylamino-acetyl)-piperazin-1-yl]-6- | | | | |
| isopropyl-5,6-dihydro-pyrrolo[3,4- | | | | |
| d]pyrimidin-7-one | | | | |
| 4-(Benzhydryl-amino)-6-dimethylamino-2- | 100 | | 1100 | |
| (2-hydroxy-ethylamino)-5,6-dihydro- | | | | |
| cyclopentapyrimidin-7-one | | | | |
| 2-(4-Ethyl-piperazin-1-yl)-6-isopropyl-4- | 101 | | 150 | |
| [(phenyl-p-tolyl-methyl)-amino]-5,6- | | | | |
| dihydro-pyrrolo[3,4-d]-pyrimidin-7-one | | | | |
| 2-(4-isopropyl-piperazin-1-yl)-6-isopropyl- | 102 | | 200 | |
| 4-[(phenyl-p-tolyl-methyl)-amino]-5,6- | | | | |
| dihydro-pyrrolo[3,4-d]-pyrimidin-7-one | | | | |
| 2-(4-Acetyl-piperazin-1-yl)-6-isopropyl-4- | 103 | | 310 | |
| {[(4-methoxy-phenyl)-phenyl-methyl]- | | | | |
| amino}-5,6-dihydro-pyrrolo[3,4- | | | | |
| d]pyrimidin-7-one | | | | |
| 2-(4-Acetyl-piperazin-1-yl)-4-(4-chloro- | 104 | | 4200 | |
| benzylamino)-6-isopropyl-5,6-dihydro- | | | | |
| pyrrolo[3,4-d]-pyrimidin-7-one | | | | |
| 2-(4-Acetyl-piperazin-1-yl)-6-isopropyl- | 105 | | 9300 | |
| 4-(3-isopropyl-phenyl amino)--5,6- | | | | |
| dihydro-pyrrolo[3,4-d]-pyrimidin-7-one | | | | |
| 4-Dibenzylamino-2-(2-dimethylamino- | 106 | | 5300 | |
| ethylamino)-6-isopropyl-5,6-dihydro- | | | | |
| pyrrolo[3,4-d]-pyrimidin-7-one | | | | |
| 4-(2,2-Diphenyl-ethylamino)-2-(4-ethyl- | 107 | | 2300 | |
| piperazin-1-yl)-6-isopropyl-5,6-dihydro- | | | | |
| pyrrolo[3,4-d]pyrimidin-7-one | | | | |
| 2-(3-Dimethylamino-propylamino)-4-(2,2- | 108 | | 4400 | |
| diphenyl-ethylamino)-6-isopropyl-5,6- | | | | |
| dihydro-pyrrolo[3,4-d]pyrimidin-7-one | | | | |
| 4-(2,2-Diphenyl-ethylamino)-6-isopropyl- | 109 | | 7200 | |
| 2-(2-methylamino-ethylamino)-5,6- | | | | |
| dihydro-pyrrolo[3,4-d]pyrimidin-7-one | | | | |
| 4-[(diphenylmethyl)amino]-2-morpholin-4- | 110 | | 3300 | |
| yl-6-propyl-5,6-dihydro-7H-pyrrolo[3,4- | | | | |
| d]pyrimidin-7-one | | | | |
| 2-(4-acetylpiperazin-1-yl)-4-[(4- | 111 | | 9000 | |
| chlorobenzyl)(methyl)amino]-6-isopropyl- | | | | |
| 5,6-dihydro-7H-pyrrolo[3,4-d]pyrimidin-7- | | | | |
| one | | | | |
| 2-(4-acetylpiperazin-1-yl)-6-isopropyl-4- | 112 | 48.42 | 65 | 1426 |
| ({(1R)-1-[4- | | | | |
| (trifluoromethoxy)phenyl]ethyl}amino)- | | | | |
| 5,6-dihydro-7H-pyrrolo[3,4-d]pyrimidin-7- | | | | |
| one | | | | |
| 2-(4-acetylpiperazin-1-yl)-4-({[1-(4- | 113 | | 460 | |
| chlorophenyl)cyclopentyl]methyl}amino)- | | | | |
| 6-isopropyl-5,6-dihydro-7H-pyrrolo[3,4- | | | | |
| d]pyrimidin-7-one | | | | |
| 2-(4-acetylpiperazin-1-yl)-4-({(1R)-1-[4- | 114 | | 71 | 3300 |
| (difluoromethoxy)phenyl]ethyl}amino)-6- | | | | |
| isopropyl-5,6-dihydro-7H-pyrrolo[3,4- | | | | |
| d]pyrimidin-7-one | | | | |
| 2-(4-acetylpiperazin-1-yl)-4-{[(1R)-1-(4- | 115 | 108.4 | 69 | 3182 |
| ethoxyphenyl)ethyl]amino}-6-isopropyl- | | | | |
| 5,6-dihydro-7H-pyrrolo[3,4-d]pyrimidin-7- | | | | |
| one | | | | |
| 2-(4-acetylpiperazin-1-yl)-4-[(2R)-2- | 116 | | 57 | 10330 |
| benzylpyrrolidin-1-yl]-6-isopropyl-5,6- | | | | |
| dihydro-7H-pyrrolo[3,4-d]pyrimidin-7-one | | | | |
| 2-(4-acetylpiperazin-1-yl)-4-[(2S)-2- | 117 | | 3700 | |
| benzylpyrrolidin-1-yl]-6-isopropyl-5,6- | | | | |
| dihydro-7H-pyrrolo[3,4-d]pyrimidin-7-one | | | | |
| 2-(4-acetylpiperazin-1-yl)-6-ethyl-4-{[2- | 118 | | 1100 | 10000 |
| (4-fluorophenyl)-1,1- | | | | |
| dimethylethyl]amino}-5,6-dihydro-7H- | | | | |
| pyrrolo[3,4-d]pyrimidin-7-one | | | | |
| 2-(4-ethylpiperazin-1-yl)-6-isopropyl-4- | 119 | | 3200 | |
| {[phenyl(pyridin-2-yl)methyl]amino}-5,6- | | | | |
| dihydro-7H-pyrrolo[3,4-d]pyrimidin-7-one | | | | |
| 2-(4-ethylpiperazin-1-yl)-4-[(9-fluoro- | 120 | | 1400 | |
| 10,11-dihydro-5H- | | | | |
| benzo[4,5]cyclohepta[1,2-b]pyridin-5- | | | | |
| yl)amino]-6-isopropyl-5,6-dihydro-7H- | | | | |
| pyrrolo[3,4-d]pyrimidin-7-one | | | | |
| 2-[[2- | 121 | | 150 | 4578 |
| (dimethylamino)ethyl](methyl)amino]-4- | | | | |
| [(9-fluoro-10,11-dihydro-5H- | | | | |
| benzo[4,5]cyclohepta[1,2-b]pyridin-5- | | | | |
| yl)amino]-6-isopropyl-5,6-dihydro-7H- | | | | |
| pyrrolo[3,4-d]pyrimidin-7-one | | | | |
| 2-(4-acetylpiperazin-1-yl)-6-isopropyl-4- | 122 | | 130 | 6324 |
| {[2-methyl-4- | | | | |
| (trifluoromethoxy)benzyl]amino}-5,6- | | | | |
| dihydro-7H-pyrrolo[3,4-d]pyrimidin-7-one | | | | |
| 2-(4-acetylpiperazin-1-yl)-4-[2-(4- | 123 | | 9100 | |
| chlorophenyl)-2-methylmorpholin-4-yl]-6- | | | | |
| isopropyl-5,6-dihydro-7H-pyrrolo[3,4- | | | | |
| d]pyrimidin-7-one | | | | |
| 4-{[2-(4-chlorophenyl)-1,1- | 124 | | 3000 | |
| dimethylethyl]amino}-2-(4- | | | | |
| isobutyrylpiperazin-1-yl)-6-isopropyl-5,6- | | | | |
| dihydro-7H-pyrrolo[3,4-d]pyrimidin-7-one | | | | |
| 4-{[2-(4-chlorophenyl)-1,1- | 125 | | 3800 | |
| dimethylethyl]amino}-2-[4-(2,2- | | | | |
| dimethylpropanoyl)piperazin-1-yl]-6- | | | | |
| isopropyl-5,6-dihydro-7H-pyrrolo[3,4- | | | | |
| d]pyrimidin-7-one | | | | |
| 4-{[2-(4-chlorophenyl)-1,1- | 126 | | 2300 | |
| dimethylethyl]amino}-2-[4- | | | | |
| (cyclopropylcarbonyl)piperazin-1-yl]-6- | | | | |
| isopropyl-5,6-dihydro-7H-pyrrolo[3,4- | | | | |
| d]pyrimidin-7-one | | | | |
| 4-(4-{[2-(4-chlorophenyl)-1,1- | 127 | | 2200 | |
| dimethylethyl]amino}-6-isopropyl-7-oxo- | | | | |
| 6,7-dihydro-5H-pyrrolo[3,4-d]pyrimidin-2- | | | | |
| yl)-N,N-dimethylpiperazine-1-carboxamide | | | | |
| 4-(4-{[2-(4-chlorophenyl)-1,1- | 128 | | 280 | |
| dimethylethyl]amino}-6-isopropyl-7-oxo- | | | | |
| 6,7-dihydro-5H-pyrrolo[3,4-d]pyrimidin-2- | | | | |
| yl)piperazine-1-carbaldehyde | | | | |
| 2-(4-Acetylpiperazin-1-yl)-4-[2-(4- | 129 | | 150 | |
| fluorobenzyl)-pyrrolidin-1-yl]-6-isopropyl- | | | | |
| 5H-pyrrolo[3,4-d]pyrimidin-7(6H)-one | | | | |
| 2-(4-Acetylpiperazin-1-yl)-4-[1-(4- | 130 | | 2300 | 13330 |
| fluorophenyl)-cyclopropylamino]-6- | | | | |
| isopropyl-5H-pyrrolo[3,4-d]pyrimidin- | | | | |
| 7(6H)-one | | | | |
| 2-(4-Acetylpiperazin-1-yl)-6-isopropyl-4- | 131 | | 6000 | 40000 |
| (2-(3-methoxyphenyl) pyrrolidin-1-yl)-5H- | | | | |
| pyrrolo[3,4-d]pyrimidin-7(6H)-one | | | | |
| 2-(4-Acetylpiperazin-1-yl)-4-(2-(3- | 132 | | 1400 | 13330 |
| chlorophenyl)pyrrolidin-1-yl)-6-isopropyl- | | | | |
| 5H-pyrrolo[3,4-d]pyrimidin-7(6H)-one | | | | |
| 2-(4-Acetylpiperazin-1-yl)-4-(5-chloro-2,3- | 133 | | 4200 | |
| dihydro-1H-inden-1-ylamino)-6-isopropyl- | | | | |
| 5H-pyrrolo[3,4-d]pyrimidin-7(6H)-one | | | | |
| 4-(1-(4-Fluorophenyl)-2-methylpropan-2- | 134 | | 490 | 49810 |
| ylamino)-6-isopropyl-2-(6- | | | | |
| oxohexahydro[1,2-a]pyrazin-2(1H)-yl)- | | | | |
| 5H-pyrrolo[3,4-d]pyrimidin-7(6H)-one | | | | |
| 6-Isopropyl-2-(6- | 135 | | 870 | |
| oxohexahydropyrrolo[1,2-a]pyrazin-2(1H)- | | | | |
| yl)-4-(quinolin-3-ylmethylamino)-5H- | | | | |
| pyrrolo[3,4-d]pyrimidin-7(6H)-one | | | | |
| 6-Isopropyl-2-(6- | 136 | | 6700 | |
| oxohexahydropyrrolo[1,2-a]pyrazin-2(1H)- | | | | |
| yl)-4-(quinolin-3-ylmethylamino)-5H- | | | | |
| pyrrolo[3,4-d]pyrimidin-7(6H)-one | | | | |
| 2-(5,6-Dihydro-[1,2,4]triazolo[4,3- | 137 | | 680 | |
| a]pyrazin-7(8H)-yl)-6-isopropyl-4- | | | | |
| (quinolin-3-ylmethylamino)-5H- | | | | |
| pyrrolo[3,4-d]pyrimidin-7(6H)-one | | | | |
| 2-(4-Acetyl-3-methyl-piperazin-1-yl)-4-[2- | 138 | | 750 | 16250 |
| (4-fluoro-phenyl)-1,1-dimethyl- | | | | |
| ethylamino]-6-isopropyl-5,6-dihydro- | | | | |
| pyrrolo[3,4-d]pyrimidin-7-one | | | | |
| 2-(4-Acetyl-2-methyl-piperazin-1-yl)-4-[2- | 139 | | 1000 | |
| (4-fluoro-phenyl)-1,1-dimethyl- | | | | |
| ethylamino]-6-isopropyl-5,6-dihydro- | | | | |
| pyrrolo[3,4-d]pyrimidin-7-one | | | | |
| (R)-7-{4-[2-(4-Fluoro-phenyl)-1,1- | 140 | | 1100 | 8471 |
| dimethyl-ethylamino]-6-isopropyl-7-oxo- | | | | |
| 6,7-dihydro-5H-pyrrolo[3,4-d]pyrimidin-2- | | | | |
| yl}-hexahydro-oxazolo[3,4-a]pyrazin-3- | | | | |
| one | | | | |
| (R)-2-(Hexahydro-pyrrolo[1,2-a]pyrazin-2- | 141 | | 7600 | |
| yl)-6-isopropyl-4-[(quinolin-3-ylmethyl)- | | | | |
| amino]-5,6-dihydro-pyrrolo[3,4- | | | | |
| d]pyrimidin-7-one | | | | |
| 2-(4-Acetyl-3-methyl-piperazin-1-yl)-6- | 142 | | 57 | 7549 |
| isopropyl-4-[(quinolin-3-ylmethyl)-amino]- | | | | |
| 5,6-dihydro-pyrrolo[3,4-d]pyrimidin-7-one | | | | |
| 4-(4-acetylpiperazin-1-yl)-8-propan-2-yl-2- | 143 | | 160 | 90000 |
| [(5-tert-butyl-1H-pyrazol-3- | | | | |
| yl)methylamino]-3,5,8- | | | | |
| triazabicyclo[4.3.0]nona-1,3,5-trien-7-one | | | | |
| 4-(4-acetylpiperazin-1-yl)-2-[(5-phenyl1,2- | 144 | | 45 | 4798 |
| oxazol-3-yl)methylamino]-8-propan-2-yl- | | | | |
| 3,5,8-triazabicyclo[4.3.0]nona-1,3,5-trien- | | | | |
| 7-one | | | | |
| 4-(4-acetylpiperazin-1-yl)-2-[(3-phenyl- | 145 | | 3200 | |
| 1,2,4-oxadiazol-5-yl)methylamino]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[(1- | 146 | | 67 | 2435 |
| phenylpyrazol-4-yl)methylamino]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[(3-phenyl1,2- | 147 | | 41 | 1229 |
| oxazol-5-yl)methylamino]-8-propan-2-yl- | | | | |
| 3,5,8-triazabicyclo[4.3.0]nona-1,3,5-trien- | | | | |
| 7-one | | | | |
| 4-(4-acetylpiperazin-1-yl)-2-[(5-phenyl- | 148 | | 160 | 6753 |
| 1,3,4-oxadiazol-2-yl)methylamino]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[1-[1-(2- | 149 | | 260 | 5066 |
| fluorophenyl)pyrazol-4-yl]ethylamino]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[1-(1- | 150 | | 60 | 236.7 |
| phenylpyrazol-4-yl)ethylamino]-8-propan- | | | | |
| 2-yl-3,5,8-triazabicyclo[4.3.0]nona-1,3,5- | | | | |
| trien-7-one | | | | |
| 4-(4-acetylpiperazin-1-yl)-2-(6,7- | 151 | | 940 | |
| diazabicyclo[3.3.0]octa-7,9-dien-8- | | | | |
| ylmethylamino)-8-propan-2-yl-3,5,8- | | | | |
| triazabicyclo[4.3.0]nona-1,3,5-trien-7-one | | | | |
| 4-(4-acetylpiperazin-1-yl)-2-[(1- | 152 | | 270 | 30000 |
| cyclopentyl-3-methyl-pyrazol-4- | | | | |
| yl)methylamino]-8-propan-2-yl-3,5,8- | | | | |
| triazabicyclo[4.3.0]nona-2,4,10-trien-7-one | | | | |
| 4-(4-acetylpiperazin-1-yl)-2-[(1-methyl-5- | 153 | | 120 | 10000 |
| phenyl-pyrazol-3-yl)methylamino]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[(2-methyl-5- | 154 | | 160 | 10000 |
| phenyl-pyrazol-3-yl)methylamino]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-(1,7- | 155 | | 500 | |
| diazabicyclo[4.3.0]nona-2,4,6,8-tetraen-8- | | | | |
| ylmethylamino)-8-propan-2-yl-3,5,8- | | | | |
| triazabicyclo[4.3.0]nona-1,3,5-trien-7-one | | | | |
| (R)-2-(4-acetylpiperazin-1-yl)-4-(1-(3- | 156 | | 830 | |
| ethoxyphenyl)ethylamino)-6-isopropyl-5H- | | | | |
| pyrrolo[3,4-d]pyrimidin-7(6H)-one | | | | |
| 2-(4-acetylpiperazin-1-yl)-4-((1-(4- | 157 | | 390 | |
| ethoxyphenyl)ethyl)(methyl)amino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 158 | | 38 | 3333 |
| ethoxy-3-fluorophenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3- | 159 | | 37 | 547.2 |
| chloro-4-ethoxyphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(2,3- | 160 | | 320 | |
| dihydrobenzofuran-5-yl)ethylamino)-6- | | | | |
| isopropyl-5H-pyrrolo[3,4-d]pyrimidin- | | | | |
| 7(6H)-one | | | | |
| (R)-2-(4-(1-(2-(4-acetylpiperazin-1-yl)-6- | 161 | | 720 | |
| isopropyl-7-oxo-6,7-dihydro-5H- | | | | |
| pyrrolo[3,4-d]pyrimidin-4- | | | | |
| ylamino)ethyl)phenyl)-2- | | | | |
| methylpropanenitrile | | | | |
| 2-(4-acetylpiperazin-1-yl)-4- | 162 | | 950 | |
| ((cyclopropylmethyl)(4- | | | | |
| ethoxybenzyl)amino)-6-isopropyl-5H- | | | | |
| pyrrolo[3,4-d]pyrimidin-7(6H)-one | | | | |
| 2-(4-(1-(2-(4-acetylpiperazin-1-yl)-6- | 163 | | 1700 | |
| isopropyl-7-oxo-6,7-dihydro-5H- | | | | |
| pyrrolo[3,4-d]pyrimidin-4-ylamino)ethyl)- | | | | |
| 2-fluorophenyl)-2-methylpropanenitrile | | | | |
| 2-(4-acetylpiperazin-1-yl)-6-isopropyl-4- | 164 | | 56 | 2909 |
| (2-methyl-1-p-tolylpropan-2-ylamino)-5H- | | | | |
| pyrrolo[3,4-d]pyrimidin-7(6H)-one | | | | |
| (R)-2-(4-acetylpiperazin-1-yl)-4-(1-(4- | 165 | | 43 | 11080 |
| iodophenyl)ethylamino)-6-isopropyl-5H- | | | | |
| pyrrolo[3,4-d]pyrimidin-7(6H)-one | | | | |
| (R)-2-(4-acetylpiperazin-1-yl)-6-isopropyl- | 166 | | 47 | 1900 |
| 4-(1-(5,6,7,8-tetrahydronaphthalen-2- | | | | |
| yl)ethylamino)-5H-pyrrolo[3,4- | | | | |
| d]pyrimidin-7(6H)-one | | | | |
| (R)-2-(4-acetylpiperazin-1-yl)-4-(1-(4- | 167 | | 65 | 3333 |
| ethoxy-3-methylphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 168 | | 80 | 3333 |
| ethoxy-2-methylphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(2,2- | 169 | | 51 | 3333 |
| dimethylchroman-6-yl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3,4- | 170 | | 96 | 10000 |
| dimethylphenyl)ethylamino)-6-isopropyl- | | | | |
| 5H-pyrrolo[3,4-d]pyrimidin-7(6H)-one | | | | |
| (R)-2-(4-acetylpiperazin-1-yl)-4-(1-(4- | 171 | | 290 | |
| chloro-3- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(2,3- | 172 | | 44 | 3333 |
| dihydro-1H-inden-5-yl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(2- | 173 | | 5400 | |
| ethoxyphenyl)ethylamino)-6-isopropyl-5H- | | | | |
| pyrrolo[3,4-d]pyrimidin-7(6H)-one | | | | |
| (R)-2-(4-acetylpiperazin-1-yl)-4-(1-(3- | 174 | | 600 | |
| ethoxy-4-methylphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 175 | | 210 | |
| ethoxyphenyl)propylamino)-6-isopropyl- | | | | |
| 5H-pyrrolo[3,4-d]pyrimidin-7(6H)-one | | | | |
| (R)-2-(4-acetylpiperazin-1-yl)-4-(1-(4- | 176 | | 80 | 3333 |
| isopropoxyphenyl)ethylamino)-6- | | | | |
| isopropyl-5H-pyrrolo[3,4-d]pyrimidin- | | | | |
| 7(6H)-one | | | | |
| (S)-2-(4-acetylpiperazin-1-yl)-4-(1-(4- | 177 | | 110 | |
| ethoxyphenyl)-2,2,2-trifluoroethylamino)- | | | | |
| 6-isopropyl-5H-pyrrolo[3,4-d]pyrimidin- | | | | |
| 7(6H)-one | | | | |
| 2-(4-acetylpiperazin-1-yl)-4-(1-(2,3- | 178 | | 970 | |
| dihydrobenzo[b][1,4]dioxin-2- | | | | |
| yl)ethylamino)-6-isopropyl-5H- | | | | |
| pyrrolo[3,4-d]pyrimidin-7(6H)-one (Isomer | | | | |
| 1) | | | | |
| 2-(4-acetylpiperazin-1-yl)-4-(1-(2,3- | 179 | | 190 | |
| dihydrobenzo[b][1,4]dioxin-2- | | | | |
| yl)ethylamino)-6-isopropyl-5H- | | | | |
| pyrrolo[3,4-d]pyrimidin-7(6H)-one (Isomer | | | | |
| 2) | | | | |
| (R)-2-(4-acetylpiperazin-1-yl)-4-(1-(4- | 180 | 7120 | 5800 | |
| ethoxyphenyl)-2-hydroxyethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3- | 181 | 174.8 | 320 | |
| (difluoromethoxy)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3- | 182 | 270.8 | 240 | |
| fluoro-4- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(2- | 183 | 198.6 | 170 | |
| fluoro-5- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3- | 184 | 100.7 | 87 | |
| fluoro-5- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4- | 185 | 79.72 | 74 | |
| (cyclopropyl(4- | | | | |
| ethoxyphenyl)methylamino)-6-isopropyl- | | | | |
| 5H-pyrrolo[3,4-d]pyrimidin-7(6H)-one | | | | |
| (R)-2-(4-acetylpiperazin-1-yl)-4-(1-(2- | 186 | 137.9 | 100 | |
| fluoro-4- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(2- | 187 | 117.1 | 96 | |
| fluoro-3- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 188 | 173.6 | 140 | |
| fluoro-3- | | | | |
| (trifluoromethyl)phenyl)ethylamino)-6- | | | | |
| isopropyl-5H-pyrrolo[3,4-d]pyrimidin- | | | | |
| 7(6H)-one | | | | |
| (S)-2-(4-acetylpiperazin-1-yl)-4-(1-(4- | 189 | 76.56 | 79 | |
| ethoxyphenyl)-2-hydroxyethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3- | 190 | 47.19 | 44 | |
| fluoro-4-isopropoxyphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 191 | 81.28 | 62 | |
| cyclobutoxyphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 192 | 101.9 | | |
| cyclopropylphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(3- | 193 | 234.9 | | |
| fluoro-4-propoxyphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(5- | 194 | 282.9 | | |
| chloro-6-ethoxypyridin-3-yl)ethylamino)- | | | | |
| 6-isopropyl-5H-pyrrolo[3,4-d]pyrimidin- | | | | |
| 7(6H)-one | | | | |
| 2-(4-acetylpiperazin-1-yl)-6-isopropyl-4- | 195 | | 18000 | |
| (1-(2-methoxyphenyl)-2-methylpropan-2- | | | | |
| ylamino)-5H-pyrrolo[3,4-d]pyrimidin- | | | | |
| 7(6H)-one | | | | |
| 2-(4-acetylpiperazin-1-yl)-6-isopropyl-4- | 196 | | 90 | 2184 |
| (1-(4-methoxyphenyl)-2-methylpropan-2- | | | | |
| ylamino)-5H-pyrrolo[3,4-d]pyrimidin- | | | | |
| 7(6H)-one | | | | |
| 2-(4-acetylpiperazin-1-yl)-6-isopropyl-4- | 197 | | 1900 | |
| (2-methyl-1-o-tolylpropan-2-ylamino)-5H- | | | | |
| pyrrolo[3,4-d]pyrimidin-7(6H)-one | | | | |
| 2-(4-acetylpiperazin-1-yl)-4-(1-(4- | 198 | 1234 | 640 | |
| ethoxyphenyl)-2-methylpropan-2- | | | | |
| ylamino)-6-isopropyl-5H-pyrrolo[3,4- | | | | |
| d]pyrimidin-7(6H)-one | | | | |
| 2-(4-acetylpiperazin-1-yl)-4- | 199 | 5045 | 2300 | |
| (benzo[d]thiazol-2-ylmethylamino)-6- | | | | |
| isopropyl-5H-pyrrolo[3,4-d]pyrimidin- | | | | |
| 7(6H)-one | | | | |
| 6-isopropyl-4-((isoquinolin-3- | 200 | 501.1 | 790 | |
| ylmethyl)(methyl)amino)-2-(4-methyl-3- | | | | |
| oxopiperazin-1-yl)-5H-pyrrolo[3,4- | | | | |
| d]pyrimidin-7(6H)-one | | | | |
| (R)-4-(1-(4-ethoxy-3- | 201 | 722.6 | 410 | |
| fluorophenyl)ethylamino)-6-isopropyl-2- | | | | |
| (4-methyl-3-oxopiperazin-1-yl)-5H- | | | | |
| pyrrolo[3,4-d]pyrimidin-7(6H)-one | | | | |
| (R)-2-(4-acetylpiperazin-1-yl)-4-(1-(4- | 202 | 46.97 | 51 | |
| ethoxy-2-methoxyphenyl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(4- | 203 | 65.84 | 62 | |
| isopropoxy-2-methoxyphenyl)ethylamino)- | | | | |
| 6-isopropyl-5H-pyrrolo[3,4-d]pyrimidin- | | | | |
| 7(6H)-one | | | | |
| (R)-2-(4-acetylpiperazin-1-yl)-4-(1-(4- | 204 | 50.45 | 38 | |
| ethoxy-2-fluorophenyl)ethylamino)-6- | | | | |
| isopropyl-5H-pyrrolo[3,4-d]pyrimidin- | | | | |
| 7(6H)-one | | | | |
| 2-(4-acetylpiperazin-1-yl)-4-(isochroman- | 205 | | 5300 | |
| 1-ylmethylamino)-6-isopropyl-5H- | | | | |
| pyrrolo[3,4-d]pyrimidin-7(6H)-one | | | | |
| 6-isopropyl-2-(3-oxopiperazin-1-yl)-4- | 206 | | 3200 | |
| (quinolin-3-ylmethylamino)-5H- | | | | |
| pyrrolo[3,4-d]pyrimidin-7(6H)-one | | | | |
| N-(1-(6-isopropyl-7-oxo-4-(quinolin-3- | 207 | | 7100 | |
| ylmethylamino)-6,7-dihydro-5H- | | | | |
| pyrrolo[3,4-d]pyrimidin-2-yl)pyrrolidin-3- | | | | |
| yl)acetamide | | | | |
| N-(1-(6-isopropyl-7-oxo-4-(quinolin-3- | 208 | | 680 | 17630 |
| ylmethylamino)-6,7-dihydro-5H- | | | | |
| pyrrolo[3,4-d]pyrimidin-2-yl)pyrrolidin-3- | | | | |
| yl)-N-methylacetamide | | | | |
| 1-(6-isopropyl-7-oxo-4-(quinolin-3- | 209 | | 2800 | |
| ylmethylamino)-6,7-dihydro-5H- | | | | |
| pyrrolo[3,4-d]pyrimidin-2-yl)piperidine-4- | | | | |
| carboxamide | | | | |
| 6-isopropyl-2-(4-methyl-3-oxopiperazin-1- | 210 | | 320 | 10000 |
| yl)-4-(quinolin-3-ylmethylamino)-5H- | | | | |
| pyrrolo[3,4-d]pyrimidin-7(6H)-one | | | | |
| 6-isopropyl-2-morpholino-4-(quinolin-3- | 211 | | 5100 | |
| ylmethylamino)-5H-pyrrolo[3,4- | | | | |
| d]pyrimidin-7(6H)-one | | | | |
| 6-isopropyl-2-[2- | 212 | | 4600 | |
| (morpholinomethyl)pyrrolidin-1-yl]-4-(3- | | | | |
| quinolylmethylamino)-5H-pyrrolo[3,4- | | | | |
| d]pyrimidin-7-one | | | | |
| 2-(3-dimethylaminopyrrolidin-1-yl)-6- | 213 | | 1100 | |
| isopropyl-4-(3-quinolylmethylamino)-5H- | | | | |
| pyrrolo[3,4-d]pyrimidin-7-one | | | | |
| 6-isopropyl-4-(3-quinolylmethylamino)-2- | 214 | | 2300 | |
| (quinuclidin-3-ylamino)-5H-pyrrolo[3,4- | | | | |
| d]pyrimidin-7-one | | | | |
| 6-isopropyl-2-(2- | 215 | | 2000 | |
| methylsulfonylethylamino)-4-(3- | | | | |
| quinolylmethylamino)-5H-pyrrolo[3,4- | | | | |
| d]pyrimidin-7-one | | | | |
| 2-(3,3-difluoropyrrolidin-1-yl)-6- | 216 | | 680 | |
| isopropyl-4-(3-quinolylmethylamino)-5H- | | | | |
| pyrrolo[3,4-d]pyrimidin-7-one | | | | |
| 6-isopropyl-2-(methyl-(tetrahydrofuran-2- | 217 | | 5900 | |
| ylmethyl)amino)-4-(3- | | | | |
| quinolylmethylamino)-5H-pyrrolo[3,4- | | | | |
| d]pyrimidin-7-one | | | | |
| 2-(4-dimethylamino-1-piperidyl)-6- | 218 | | 7800 | |
| isopropyl-4-(3-quinolylmethylamino)-5H- | | | | |
| pyrrolo[3,4-d]pyrimidin-7-one | | | | |
| 6-(1-methylethyl)-4-[(quinolin-3- | 219 | | 8400 | |
| ylmethyl)amino]-2-[(tetrahydro-2H-pyran- | | | | |
| 4-ylmethyl)amino]-5,6-dihydro-7H- | | | | |
| pyrrolo[3,4-d]pyrimidin-7-one | | | | |
| 2-(dimethylamino)-6-(1-methylethyl)-4- | 220 | | 9600 | |
| [(quinolin-3-ylmethyl)amino]-5,6-dihydro- | | | | |
| 7H-pyrrolo[3,4-d]pyrimidin-7-one | | | | |
| N,N-dimethyl-4-{6-(1-methylethyl)-7-oxo- | 221 | | 5800 | |
| 4-[(quinolin-3-ylmethyl)amino]-6,7- | | | | |
| dihydro-5H-pyrrolo[3,4-d]pyrimidin-2- | | | | |
| yl}piperazine-1-carboxamide | | | | |
| 2-[4-(cyclopropylcarbonyl)piperazin-1-yl]- | 222 | | 6300 | |
| 6-(1-methylethyl)-4-[(quinolin-3- | | | | |
| ylmethyl)amino]-5,6-dihydro-7H- | | | | |
| pyrrolo[3,4-d]pyrimidin-7-one | | | | |
| 2-(4-acetylpiperazin-1-yl)-4-(2-(4- | 223 | | 620 | 90000 |
| ethylpiperazin-1-yl)-4- | | | | |
| (trifluoromethyl)benzylamino)-6- | | | | |
| isopropyl-5H-pyrrolo[3,4-d]pyrimidin- | | | | |
| 7(6H)-one | | | | |
| 2-(4-acetylpiperazin-1-yl)-6-isopropyl-4- | 224 | | 4100 | |
| (1-m-tolylpyrrolidin-3-ylamino)-5H- | | | | |
| pyrrolo[3,4-d]pyrimidin-7(6H)-one | | | | |
| AZ1282806 | | | | |
| 2-(4-acetylpiperazin-1-yl)-6-isopropyl-4- | 225 | | 290 | 60750 |
| (2-methoxy-4- | | | | |
| (trifluoromethyl)benzylamino)-5H- | | | | |
| pyrrolo[3,4-d]pyrimidin-7(6H)-one | | | | |
| (R)-2-(4-acetylpiperazin-1-yl)-6-isopropyl- | 226 | | 4400 | |
| 4-(1-(2- | | | | |
| (trifluoromethyl)phenyl)ethylamino)-5H- | | | | |
| pyrrolo[3,4-d]pyrimidin-7(6H)-one | | | | |
| (R)-2-(4-acetylpiperazin-1-yl)-6-isopropyl- | 227 | | 8500 | |
| 4-(1-(3-methoxyphenyl)ethylamino)-5H- | | | | |
| pyrrolo[3,4-d]pyrimidin-7(6H)-one | | | | |
| 2-(4-acetylpiperazin-1-yl)-6-isopropyl-4- | 228 | | 8800 | |
| ((6-phenylpyridin-3-yl)methylamino)-5H- | | | | |
| pyrrolo[3,4-d]pyrimidin-7(6H)-one | | | | |
| 2-(4-acetylpiperazin-1-yl)-6-isopropyl-4- | 229 | | 120 | 14630 |
| (isoquinolin-4-ylmethylamino)-5H- | | | | |
| pyrrolo[3,4-d]pyrimidin-7(6H)-one | | | | |
| (R)-2-(4-acetylpiperazin-1-yl)-6-isopropyl- | 230 | | 87 | 7385 |
| 4-(1-(3- | | | | |
| (trifluoromethyl)phenyl)ethylamino)-5H- | | | | |
| pyrrolo[3,4-d]pyrimidin-7(6H)-one | | | | |
| 2-(4-acetylpiperazin-1-yl)-6-isopropyl-4- | 231 | | 1300 | 90000 |
| (4-(trifluoromethyl)benzylamino)-5H- | | | | |
| pyrrolo[3,4-d]pyrimidin-7(6H)-one | | | | |
| 2-(4-acetylpiperazin-1-yl)-6-isopropyl-4- | 232 | | 67 | 7844 |
| (2-methoxy-4- | | | | |
| (trifluoromethoxy)benzylamino)-5H- | | | | |
| pyrrolo[3,4-d]pyrimidin-7(6H)-one | | | | |
| (R)-2-(4-acetylpiperazin-1-yl)-6-isopropyl- | 233 | | 21 | 74.36 |
| 4-(1-(quinolin-3-yl)ethylamino)-5H- | | | | |
| pyrrolo[3,4-d]pyrimidin-7(6H)-one | | | | |
| (S)-2-(4-acetylpiperazin-1-yl)-6-isopropyl- | 234 | | 1100 | |
| 4-(1-(quinolin-3-yl)ethylamino)-5H- | | | | |
| pyrrolo[3,4-d]pyrimidin-7(6H)-one | | | | |
| 2-(4-acetylpiperazin-1-yl)-6-isopropyl-4- | 235 | | 83 | 13790 |
| (quinolin-6-ylmethylamino)-5H- | | | | |
| pyrrolo[3,4-d]pyrimidin-7(6H)-one | | | | |
| 2-(4-acetylpiperazin-1-yl)-4-(1- | 236 | 563.9 | | |
| (benzo[d]thiazol-2-yl)ethylamino)-6- | | | | |
| isopropyl-5H-pyrrolo[3,4-d]pyrimidin- | | | | |
| 7(6H)-one | | | | |
| 2-(4-acetylpiperazin-1-yl)-6-isopropyl-4- | 237 | | 500 | 30000 |
| ((1-methyl-1H-indol-2-yl)methylamino)- | | | | |
| 5H-pyrrolo[3,4-d]pyrimidin-7(6H)-one | | | | |
| 2-(4-acetylpiperazin-1-yl)-6-isopropyl-4- | 238 | 14.64 | 24 | 8579 |
| (isoquinolin-3-ylmethylamino)-5H- | | | | |
| pyrrolo[3,4-d]pyrimidin-7(6H)-one | | | | |
| 2-(4-acetylpiperazin-1-yl)-6-isopropyl-4- | 239 | | 1200 | |
| (methyl(quinolin-6-ylmethyl)amino)-5H- | | | | |
| pyrrolo[3,4-d]pyrimidin-7(6H)-one | | | | |
| 2-(4-acetylpiperazin-1-yl)-6-isopropyl-4- | 240 | | 1500 | |
| ((8-methoxyquinolin-5-yl)methylamino)- | | | | |
| 5H-pyrrolo[3,4-d]pyrimidin-7(6H)-one | | | | |
| 2-(4-acetylpiperazin-1-yl)-6-isopropyl-4- | 241 | | 68 | 30000 |
| ((2-methylquinolin-3-yl)methylamino)-5H- | | | | |
| pyrrolo[3,4-d]pyrimidin-7(6H)-one | | | | |
| 2-(4-acetylpiperazin-1-yl)-6-isopropyl-4- | 242 | | 65 | 10420 |
| ((2-methylquinolin-6-yl)methylamino)-5H- | | | | |
| pyrrolo[3,4-d]pyrimidin-7(6H)-one | | | | |
| (R)-2-(4-acetylpiperazin-1-yl)-6-isopropyl- | 243 | | 36 | 1244 |
| 4-(1-(quinolin-6-yl)ethylamino)-5H- | | | | |
| pyrrolo[3,4-d]pyrimidin-7(6H)-one | | | | |
| (S)-2-(4-acetylpiperazin-1-yl)-6-isopropyl- | 244 | | 6500 | |
| 4-(1-(quinolin-6-yl)ethylamino)-5H- | | | | |
| pyrrolo[3,4-d]pyrimidin-7(6H)-one | | | | |
| 2-(4-acetylpiperazin-1-yl)-4- | 245 | | 28 | 2161 |
| (ethyl(isoquinolin-3-ylmethyl)amino)-6- | | | | |
| isopropyl-5H-pyrrolo[3,4-d]pyrimidin- | | | | |
| 7(6H)-one | | | | |
| 2-(4-acetylpiperazin-1-yl)-4- | 246 | | 68 | 10440 |
| (ethyl(quinolin-3-ylmethyl)amino)-6- | | | | |
| isopropyl-5H-pyrrolo[3,4-d]pyrimidin- | | | | |
| 7(6H)-one | | | | |
| 2-(4-acetylpiperazin-1-yl)-6-isopropyl-4- | 247 | | 77 | 9071 |
| ((8-methylquinolin-6-yl)methylamino)-5H- | | | | |
| pyrrolo[3,4-d]pyrimidin-7(6H)-one | | | | |
| 2-(4-acetylpiperazin-1-yl)-6-isopropyl-4- | 248 | | 6300 | |
| (quinolin-2-ylmethylamino)-5H- | | | | |
| pyrrolo[3,4-d]pyrimidin-7(6H)-one | | | | |
| 2-(4-acetylpiperazin-1-yl)-6-isopropyl-4- | 249 | | 6000 | |
| (methyl(quinolin-2-ylmethyl)amino)-5H- | | | | |
| pyrrolo[3,4-d]pyrimidin-7(6H)-one | | | | |
| (R)-2-(4-acetylpiperazin-1-yl)-4-(1-(4-(1,3- | 250 | | 430 | |
| difluoropropan-2- | | | | |
| yloxy)phenyl)ethylamino)-6-isopropyl-5H- | | | | |
| pyrrolo[3,4-d]pyrimidin-7(6H)-one | | | | |
| (S)-2-(4-acetylpiperazin-1-yl)-4-(1-(4-(1,3- | 251 | | 5200 | |
| difluoropropan-2- | | | | |
| yloxy)phenyl)ethylamino)-6-isopropyl-5H- | | | | |
| pyrrolo[3,4-d]pyrimidin-7(6H)-one | | | | |
| 2-(4-acetylpiperazin-1-yl)-4-((1- | 252 | | 87 | 2110 |
| (dimethylamino)isoquinolin-3- | | | | |
| yl)methylamino)-6-isopropyl-5H- | | | | |
| pyrrolo[3,4-d]pyrimidin-7(6H)-one | | | | |
| 2-(4-acetylpiperazin-1-yl)-4- | 253 | | 63 | 1108 |
| ((cyclopropylmethyl)(isoquinolin-3- | | | | |
| ylmethyl)amino)-6-isopropyl-5H- | | | | |
| pyrrolo[3,4-d]pyrimidin-7(6H)-one | | | | |
| 2-(4-acetylpiperazin-1-yl)-6-isopropyl-4- | 254 | | 75 | 10000 |
| (isopropyl(isoquinolin-3-ylmethyl)amino)- | | | | |
| 5H-pyrrolo[3,4-d]pyrimidin-7(6H)-one | | | | |
| 2-(4-acetylpiperazin-1-yl)-6-isopropyl-4- | 255 | 35.58 | 22 | 10000 |
| ((isoquinolin-3-ylmethyl)(methyl)amino)- | | | | |
| 5H-pyrrolo[3,4-d]pyrimidin-7(6H)-one | | | | |
| 2-(4-acetylpiperazin-1-yl)-4-((2,2- | 256 | | 46 | 3333 |
| difluoroethyl)(isoquinolin-3- | | | | |
| ylmethyl)amino)-6-isopropyl-5H- | | | | |
| pyrrolo[3,4-d]pyrimidin-7(6H)-one | | | | |
| 2-(4-acetylpiperazin-1-yl)-4-(ethyl(1- | 257 | 83.18 | 80 | |
| (quinolin-6-yl)ethyl)amino)-6-isopropyl- | | | | |
| 5H-pyrrolo[3,4-d]pyrimidin-7(6H)-one | | | | |
| (Enantiomer 1) | | | | |
| 2-(4-acetylpiperazin-1-yl)-4-(ethyl(1- | 258 | 231.5 | 250 | |
| (quinolin-6-yl)ethyl)amino)-6-isopropyl- | | | | |
| 5H-pyrrolo[3,4-d]pyrimidin-7(6H)-one | | | | |
| (Enantiomer 2) | | | | |
| 2-(4-acetylpiperazin-1-yl)-6-isopropyl-4- | 259 | 27.21 | 16 | |
| ((1-methylisoquinolin-3-yl)methylamino)- | | | | |
| 5H-pyrrolo[3,4-d]pyrimidin-7(6H)-one | | | | |
| 2-(4-acetylpiperazin-1-yl)-6-isopropyl-4- | 260 | 1900 | 2300 | |
| (methyl(1-(quinolin-6-yl)ethyl)amino)-5H- | | | | |
| pyrrolo[3,4-d]pyrimidin-7(6H)-one | | | | |
| (Enantiomer 1) | | | | |
| 2-(4-acetylpiperazin-1-yl)-6-isopropyl-4- | 261 | 71.13 | 64 | |
| (methyl(1-(quinolin-6-yl)ethyl)amino)-5H- | | | | |
| pyrrolo[3,4-d]pyrimidin-7(6H)-one | | | | |
| (Enantiomer 2) | | | | |
| (R)-2-(4-acetylpiperazin-1-yl)-4-((1-(4- | 262 | 84.53 | 85 | |
| ethoxy-3- | | | | |
| fluorophenyl)ethyl)(methyl)amino)-6- | | | | |
| isopropyl-5H-pyrrolo[3,4-d]pyrimidin- | | | | |
| 7(6H)-one | | | | |
| 2-(4-acetylpiperazin-1-yl)-6-isopropyl-4- | 263 | 84.97 | 94 | |
| (methyl((1-methylisoquinolin-3- | | | | |
| yl)methyl)amino)-5H-pyrrolo[3,4- | | | | |
| d]pyrimidin-7(6H)-one | | | | |
| 2-(4-acetylpiperazin-1-yl)-6-isopropyl-4- | 264 | | 2900 | |
| ((isoquinolin-3-ylmethyl)(2,2,2- | | | | |
| trifluoroethyl)amino)-5H-pyrrolo[3,4- | | | | |
| d]pyrimidin-7(6H)-one | | | | |
| benzyl (2R)-1-[4-(4-acetylpiperazin-1-yl)- | 265 | | 850 | 15700 |
| 7-oxo-8-propan-2-yl-3,5,8- | | | | |
| triazabicyclo[4.3.0]nona-1,3,5-trien-2- | | | | |
| yl]pyrrolidine-2-carboxylate | | | | |
| 4-(4-acetylpiperazin-1-yl)-2-(2-benzyl-1- | 266 | | 1000 | |
| piperidyl)-8-propan-2-yl-3,5,8- | | | | |
| triazabicyclo[4.3.0]nona-1,3,5-trien-7-one | | | | |
| 4-(4-acetylpiperazin-1-yl)-2-[2-(4- | 267 | | 480 | 30000 |
| dimethylaminophenyl)pyrrolidin-1-yl]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[2-[(4- | 268 | | 500 | 12450 |
| fluorophenyl)methyl]-1-piperidyl]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-2-[2-(3- | 269 | | 740 | 30000 |
| methylphenyl)pyrrolidin-1-yl]-8-propan-2- | | | | |
| yl-3,5,8-triazabicyclo[4.3.0]nona-2,4,10- | | | | |
| trien-7-one | | | | |
| 4-(4-acetylpiperazin-1-yl)-2-[2-(3,5- | 270 | | 340 | 9911 |
| dimethylphenyl)pyrrolidin-1-yl]-8-propan- | | | | |
| 2-yl-3,5,8-triazabicyclo[4.3.0]nona-2,4,10- | | | | |
| trien-7-one | | | | |
| 4-(4-acetylpiperazin-1-yl)-2-(2- | 271 | | 870 | |
| phenethylpyrrolidin-1-yl)-8-propan-2-yl- | | | | |
| 3,5,8-triazabicyclo[4.3.0]nona-1,3,5-trien- | | | | |
| 7-one | | | | |
| 4-(4-acetylpiperazin-1-yl)-2-[2-(4- | 272 | | 270 | 12860 |
| ethoxyphenyl)pyrrolidin-1-yl]-8-propan-2- | | | | |
| yl-3,5,8-triazabicyclo[4.3.0]nona-2,4,10- | | | | |
| trien-7-one | | | | |
| 4-(4-acetylpiperazin-1-yl)-2-(2- | 273 | | 3200 | |
| benzylazetidin-1-yl)-8-propan-2-yl-3,5,8- | | | | |
| triazabicyclo[4.3.0]nona-1,3,5-trien-7-one | | | | |
| 4-(4-acetylpiperazin-1-yl)-8-propan-2-yl-2- | 274 | | 170 | 767.6 |
| (2-quinolin-3-ylpyrrolidin-1-yl)-3,5,8- | | | | |
| triazabicyclo[4.3.0]nona-1,3,5-trien-7-one | | | | |
| 4-(4-acetylpiperazin-1-yl)-2-[2-(1- | 275 | | 760 | 10290 |
| phenylpropyl)pyrrolidin-1-yl]-8-propan-2- | | | | |
| yl-3,5,8-triazabicyclo[4.3.0]nona-2,4,10- | | | | |
| trien-7-one | | | | |
| 4-(4-acetylpiperazin-1-yl)-2-[(3- | 276 | | 590 | 90000 |
| cyclopentyl-1,2,4-oxadiazol-5- | | | | |
| yl)methylamino]-8-propan-2-yl-3,5,8- | | | | |
| triazabicyclo[4.3.0]nona-1,3,5-trien-7-one | | | | |
| tert-butyl (2R)-1-[4-(4-acetylpiperazin-1- | 277 | | 7700 | |
| yl)-7-oxo-8-propan-2-yl-3,5,8- | | | | |
| triazabicyclo[4.3.0]nona-1,3,5-trien-2- | | | | |
| yl]pyrrolidine-2-carboxylate | | | | |
| 4-(4-acetylpiperazin-1-yl)-2-[(5- | 278 | | 750 | 90000 |
| cyclobutyl-1,2,4-oxadiazol-3- | | | | |
| yl)methylamino]-8-propan-2-yl-3,5,8- | | | | |
| triazabicyclo[4.3.0]nona-1,3,5-trien-7-one | | | | |
| 4-(4-acetylpiperazin-1-yl)-2-[(2R)-2-(2,3- | 279 | | 1800 | |
| dihydroindole-1-carbonyl)pyrrolidin-1-yl]- | | | | |
| 8-propan-2-yl-3,5,8- | | | | |
| triazabicyclo[4.3.0]nona-1,3,5-trien-7-one | | | | |
| 4-(4-acetylpiperazin-1-yl)-2-[2-(3-phenyl- | 280 | | 5100 | |
| 1,2,4-oxadiazol-5-yl)pyrrolidin-1-yl]-8- | | | | |
| propan-2-yl-3,5,8-triazabicyclo[4.3.0]nona- | | | | |
| 1,3,5-trien-7-one | | | | |
| 4-(4-acetylpiperazin-1-yl)-8-propan-2-yl-2- | 281 | | 140 | 11430 |
| (2-quinolin-6-ylpyrrolidin-1-yl)-3,5,8- | | | | |
| triazabicyclo[4.3.0]nona-1,3,5-trien-7-one | | | | |
| 4-(4-acetylpiperazin-1-yl)-8-propan-2-yl-2- | 282 | | 550 | 2527 |
| (2-quinolin-3-ylpyrrolidin-1-yl)-3,5,8- | | | | |
| triazabicyclo[4.3.0]nona-1,3,5-trien-7-one | | | | |
| (Enantiomer 1) | | | | |
| 4-(4-acetylpiperazin-1-yl)-8-propan-2-yl-2- | 283 | | 87 | 252.7 |
| (2-quinolin-3-ylpyrrolidin-1-yl)-3,5,8- | | | | |
| triazabicyclo[4.3.0]nona-1,3,5-trien-7-one | | | | |
| (Enantiomer 2) | | | | |
| 4-(4-acetylpiperazin-1-yl)-8-propan-2-yl-2- | 284 | | 9900 | |
| (2-quinolin-6-ylpyrrolidin-1-yl)-3,5,8- | | | | |
| triazabicyclo[4.3.0]nona-1,3,5-trien-7-one | | | | |
| (Enantiomer 1) | | | | |
| 4-(4-acetylpiperazin-1-yl)-8-propan-2-yl-2- | 285 | | 100 | |
| (2-quinolin-6-ylpyrrolidin-1-yl)-3,5,8- | | | | |
| triazabicyclo[4.3.0]nona-1,3,5-trien-7-one | | | | |
| (Enantiomer 2) | | | | |
| 2-(4-acetylpiperazin-1-yl)-6-isopropyl-4- | 286 | | 67 | 3432 |
| [2-[(4-methoxyphenyl)methyl]pyrrolidin-1- | | | | |
| yl]-5H-pyrrolo[3,4-d]pyrimidin-7-one | | | | |
| 2-(4-acetylpiperazin-1-yl)-6-isopropyl-4- | 287 | | 53 | 1496 |
| [2-[(4-methoxyphenyl)methyl]pyrrolidin-1- | | | | |
| yl]-5H-pyrrolo[3,4-d]pyrimidin-7-one | | | | |
| (Enantiomer 1) | | | | |
| 2-(4-acetylpiperazin-1-yl)-6-isopropyl-4- | 288 | | 1400 | |
| [2-[(4-methoxyphenyl)methyl]pyrrolidin-1- | | | | |
| yl]-5H-pyrrolo[3,4-d]pyrimidin-7-one | | | | |
| (Enantiomer 2) | | | | |
| 2-(4-acetylpiperazin-1-yl)-4-[2-[(4- | 289 | | 330 | |
| ethoxyphenyl)methyl]pyrrolidin-1-yl]-6- | | | | |
| isopropyl-5H-pyrrolo[4,3-e]pyrimidin-7- | | | | |
| one | | | | |
| 2-(4-acetylpiperazin-1-yl)-6-isopropyl-4- | 290 | | 2000 | |
| [2-[(2-methoxyphenyl)methyl]pyrrolidin-1- | | | | |
| yl]-5H-pyrrolo[3,4-d]pyrimidin-7-one | | | | |
| 2-(4-acetylpiperazin-1-yl)-6-isopropyl-4- | 291 | | 490 | |
| [2-[(3-methoxyphenyl)methyl]pyrrolidin-1- | | | | |
| yl]-5H-pyrrolo[3,4-d]pyrimidin-7-one | | | | |
| 2-(4-acetylpiperazin-1-yl)-4-[2-(4- | 292 | 4949 | 3600 | |
| fluorophenyl)pyrrolidin-1-yl]-6-(1- | | | | |
| methylethyl)-5,6-dihydro-7H-pyrrolo[3,4- | | | | |
| d]pyrimidin-7-one | | | | |
| 2-(4-acetylpiperazin-1-yl)-4-{2-[4- | 293 | 1589 | 1400 | |
| (methoxymethyl)benzyl]pyrrolidin-1-yl}- | | | | |
| 6-(1-methylethyl)-5,6-dihydro-7H- | | | | |
| pyrrolo[3,4-d]pyrimidin-7-one | | | | |
| 4-[2-(4-acetylbenzyl)pyrrolidin-1-yl]-2-(4- | 294 | 1845 | 980 | |
| acetylpiperazin-1-yl)-6-(1-methylethyl)- | | | | |
| 5,6-dihydro-7H-pyrrolo[3,4-d]pyrimidin-7- | | | | |
| one | | | | |
| 2-(4-acetylpiperazin-1-yl)-4-[2-(4- | 295 | 824.3 | 730 | |
| methoxy-3-methylphenyl)pyrrolidin-1-yl]- | | | | |
| 6-(1-methylethyl)-5,6-dihydro-7H- | | | | |
| pyrrolo[3,4-d]pyrimidin-7-one | | | | |
| 2-(4-acetylpiperazin-1-yl)-6-(1- | 296 | 1194 | 1400 | |
| methylethyl)-4-[2-(4- | | | | |
| methylphenyl)pyrrolidin-1-yl]-5,6-dihydro- | | | | |
| 7H-pyrrolo[3,4-d]pyrimidin-7-one | | | | |
| 2-(4-acetylpiperazin-1-yl)-4-[2-(3,4- | 297 | 322.4 | 300 | |
| dichlorophenyl)pyrrolidin-1-yl]-6-(1- | | | | |
| methylethyl)-5,6-dihydro-7H-pyrrolo[3,4- | | | | |
| d]pyrimidin-7-one | | | | |
| 2-(4-acetylpiperazin-1-yl)-4-[2-(3,4- | 298 | 453.8 | | |
| dimethylphenyl)pyrrolidin-1-yl]-6-(1- | | | | |
| methylethyl)-5,6-dihydro-7H-pyrrolo[3,4- | | | | |
| d]pyrimidin-7-one | | | | |
| 2-(4-acetylpiperazin-1-yl)-4-[2-(4- | 299 | 1145 | | |
| methoxyphenyl)pyrrolidin-1-yl]-6-(1- | | | | |
| methylethyl)-5,6-dihydro-7H-pyrrolo[3,4- | | | | |
| d]pyrimidin-7-one | | | | |
| 2-(4-acetylpiperazin-1-yl)-4-[(2R)-2-(4- | 300 | 192.2 | | |
| ethoxy-3-fluorophenyl)pyrrolidin-1-yl]-6- | | | | |
| (1-methylethyl)-5,6-dihydro-7H- | | | | |
| pyrrolo[3,4-d]pyrimidin-7-one | | | | |
| (4-chlorophenyl)methyl (2R)-1-[4-(4- | 301 | | 2500 | |
| acetylpiperazin-1-yl)-7-oxo-8-propan-2-yl- | | | | |
| 3,5,8-triazabicyclo[4.3.0]nona-1,3,5-trien- | | | | |
| 2-yl]pyrrolidine-2-carboxylate | | | | |
| (2R)-1-[4-(4-acetylpiperazin-1-yl)-7-oxo-8- | 302 | | 3100 | |
| propan-2-yl-3,5,8-triazabicyclo[4.3.0]nona- | | | | |
| 1,3,5-trien-2-yl]-N-cyclohexyl-pyrrolidine- | | | | |
| 2-carboxamide | | | | |
| 4-(4-acetylpiperazin-1-yl)-2-[(2R)-2-(4- | 303 | | 2300 | |
| methylpiperidine-1-carbonyl)pyrrolidin-1- | | | | |
| yl]-8-propan-2-yl-3,5,8- | | | | |
| triazabicyclo[4.3.0]nona-2,4,10-trien-7-one | | | | |
| phenyl (2R)-1-[4-(4-acetylpiperazin-1-yl)- | 304 | | 71 | 8734 |
| 7-oxo-8-propan-2-yl-3,5,8- | | | | |
| triazabicyclo[4.3.0]nona-1,3,5-trien-2- | | | | |
| yl]pyrrolidine-2-carboxylate | | | | |
| 3-(4-acetylpiperazin-1-yl)-5-[[1-(4- | 305 | | 2400 | |
| fluorophenyl)-2-methyl-propan-2- | | | | |
| yl]amino]-8-(oxan-4-yl)-2,4,8- | | | | |
| triazabicyclo[4.3.0]nona-1,3,5-trien-9-one | | | | |
| (+)-4-(4-acetylpiperazin-1-yl)-8-butan-2- | 306 | | 34 | 3972 |
| yl-2-[(1-phenylpyrazol-4-yl)methylamino]- | | | | |
| 3,5,8-triazabicyclo[4.3.0]nona-1,3,5-trien- | | | | |
| 7-one (Enantiomer 1) | | | | |
| (−)-4-(4-acetylpiperazin-1-yl)-8-butan-2-yl- | 307 | | 150 | 11970 |
| 2-[(1-phenylpyrazol-4-yl)methylamino]- | | | | |
| 3,5,8-triazabicyclo[4.3.0]nona-1,3,5-trien- | | | | |
| 7-one (Enantiomer 2) | | | | |
| (+)-2-(4-acetylpiperazin-1-yl)-4-(3- | 308 | | 20 | 10310 |
| quinolylmethylamino)-6-sec-butyl-5H- | | | | |
| pyrrolo[3,4-d]pyrimidin-7-one (Enantiomer | | | | |
| 1) | | | | |
| (−)-2-(4-acetylpiperazin-1-yl)-4-(3- | 309 | | 42 | 10000 |
| quinolylmethylamino)-6-sec-butyl-5H- | | | | |
| pyrrolo[3,4-d]pyrimidin-7-one (Enantiomer | | | | |
| 2) | | | | |
| (+)-2-(4-acetylpiperazin-1-yl)-4-(3- | 310 | | 29 | 3333 |
| isoquinolylmethylamino)-6-sec-butyl-5H- | | | | |
| pyrrolo[3,4-d]pyrimidin-7-one (Enatiomer | | | | |
| 1) | | | | |
| (−)-2-(4-acetylpiperazin-1-yl)-4-(3- | 311 | | 28 | 1678 |
| isoquinolylmethylamino)-6-sec-butyl-5H- | | | | |
| pyrrolo[3,4-d]pyrimidin-7-one (Enatiomer | | | | |
| 2) | | | | |
| 2-(4-acetylpiperazin-1-yl)-6-sec-butyl-4- | 312 | | 160 | 10000 |
| (1-(4-fluorophenyl)-2-methylpropan-2- | | | | |
| ylamino)-5H-pyrrolo[3,4-d]pyrimidin- | | | | |
| 7(6H)-one | | | | |
| 2-(4-acetylpiperazin-1-yl)-4-(1-(4- | 313 | | 280 | 10000 |
| fluorophenyl)-2-methylpropan-2-ylamino)- | | | | |
| 6-(3-methylbutan-2-yl)-5H-pyrrolo[3,4- | | | | |
| d]pyrimidin-7(6H)-one | | | | |
| 2-(4-acetylpiperazin-1-yl)-4-(1-(4- | 314 | | 670 | 30000 |
| fluorophenyl)-2-methylpropan-2-ylamino)- | | | | |
| 6-(1-methoxypropan-2-yl)-5H-pyrrolo[3,4- | | | | |
| d]pyrimidin-7(6H)-one | | | | |
| 2-(4-acetylpiperazin-1-yl)-6-(2- | 315 | | 3000 | |
| chlorobenzyl)-4-(1-(4-fluorophenyl)-2- | | | | |
| methylpropan-2-ylamino)-5H-pyrrolo[3,4- | | | | |
| d]pyrimidin-7(6H)-one | | | | |
| 2-(4-acetylpiperazin-1-yl)-6-[(1S)-1- | 316 | 35.53 | 28 | |
| methylpropyl]-4-[(quinolin-3- | | | | |
| ylmethyl)amino]-5,6-dihydro-7H- | | | | |
| pyrrolo[3,4-d]pyrimidin-7-one | | | | |
| 2-(4-acetylpiperazin-1-yl)-4- | 317 | 39.97 | 32 | |
| [ethyl(isoquinolin-3-ylmethyl)amino]-6-(1- | | | | |
| methylpropyl)-5,6-dihydro-7H-pyrrolo[3,4- | | | | |
| d]pyrimidin-7-one (Enantiomer 1) | | | | |
| 2-(4-acetylpiperazin-1-yl)-4- | 318 | 43.03 | 41 | |
| [ethyl(isoquinolin-3-ylmethyl)amino]-6-(1- | | | | |
| methylpropyl)-5,6-dihydro-7H-pyrrolo[3,4- | | | | |
| d]pyrimidin-7-one (Enantiomer 2) | | | | |
| 2-(4-acetylpiperazin-1-yl)-6-[(1S)-1- | 319 | 53.59 | 47 | |
| methylpropyl]-4-[methyl(quinolin-3- | | | | |
| ylmethyl)amino]-5,6-dihydro-7H- | | | | |
| pyrrolo[3,4-d]pyrimidin-7-one | | | | |
| 2-(4-acetylpiperazin-1-yl)-4-{[(1R)-1-(4- | 320 | 69.07 | 54 | |
| ethoxy-3-fluorophenyl)ethyl]amino}-6- | | | | |
| [(1S)-1-methylpropyl]-5,6-dihydro-7H- | | | | |
| pyrrolo[3,4-d]pyrimidin-7-one | | | | |
| (R)-2-(4-acetylpiperazin-1-yl)-4-(1-(4- | 321 | 3357 | 2900 | |
| ethoxyphenyl)-2,2,2-trifluoroethylamino)- | | | | |
| 6-isopropyl-5H-pyrrolo[3,4-d]pyrimidin- | | | | |
| 7(6H)-one | | | | |
| (R)-2-(4-acetylpiperazin-1-yl)-4-(1-(4- | 322 | 1897 | | |
| (cyclopropylmethoxy)-3- | | | | |
| fluorophenyl)ethylamino)-6-isopropyl-5H- | | | | |
| pyrrolo[3,4-d]pyrimidin-7(6H)-one | | | | |
| 2-(4-acetylpiperazin-1-yl)-6-isopropyl-4- | 323 | 410.5 | | |
| ((2-methyl-2,3-dihydrobenzofuran-5- | | | | |
| yl)methylamino)-5H-pyrrolo[3,4- | | | | |
| d]pyrimidin-7(6H)-one | | | | |
| 2-(4-acetylpiperazin-1-yl)-6-isopropyl-4- | 324 | 179.42 | | |
| (1-(2-methyl-2,3-dihydrobenzofuran-5- | | | | |
| yl)ethylamino)-5H-pyrrolo[3,4- | | | | |
| d]pyrimidin-7(6H)-one | | | | |
| (S)—N-(2-(4-acetyl-3-methylpiperazin-1- | 325 | | 380 | |
| yl)-6-isopropyl-7-oxo-6,7-dihydro-5H- | | | | |
| pyrrolo[3,4-d]pyrimidin-4-yl)-N- | | | | |
| (isoquinolin-3-ylmethyl)acetamide | | | | |
| (R)—N-(2-(4-acetyl-3-methylpiperazin-1- | 326 | | 590 | |
| yl)-6-isopropyl-7-oxo-6,7-dihydro-5H- | | | | |
| pyrrolo[3,4-d]pyrimidin-4-yl)-N- | | | | |
| (isoquinolin-3-ylmethyl)acetamide | | | | |
| (S)-2-(4-acetyl-3-methylpiperazin-1-yl)-6- | 327 | | 91 | 30000 |
| isopropyl-4-(quinolin-3-ylmethylamino)- | | | | |
| 5H-pyrrolo[3,4-d]pyrimidin-7(6H)-one | | | | |
| (R)-2-(4-acetyl-3-methylpiperazin-1-yl)-6- | 328 | | 62 | 30000 |
| isopropyl-4-(quinolin-3-ylmethylamino)- | | | | |
| 5H-pyrrolo[3,4-d]pyrimidin-7(6H)-one | | | | |
| 6-(1-methylethyl)-2-[4- | 329 | | 8900 | |
| (methylsulfonyl)piperazin-1-yl]-4- | | | | |
| [(quinolin-3-ylmethyl)amino]-5,6-dihydro- | | | | |
| 7H-pyrrolo[3,4-d]pyrimidin-7-one | | | | |
| (R)-2-(4-acetylpiperazin-1-yl)-4-(1-(6- | 330 | 382.1 | | |
| ethoxy-5-fluoropyridin-3-yl)ethylamino)-6- | | | | |
| isopropyl-5H-pyrrolo[3,4-d]pyrimidin- | | | | |
| 7(6H)-one | | | | |
| (R)-2-(4-acetylpiperazin-1-yl)-4-(1-(6- | 331 | 195.3 | | |
| ethoxy-5-methylpyridin-3-yl)ethylamino)- | | | | |
| 6-isopropyl-5H-pyrrolo[3,4-d]pyrimidin- | | | | |
| 7(6H)-one | | | | |
| (R)-2-(4-acetylpiperazin-1-yl)-4-(1-(4- | 332 | >30000 | | |
| (hydroxymethyl)phenyl)ethylamino)-6- | | | | |
| isopropyl-5H-pyrrolo[3,4-d]pyrimidin- | | | | |
| 7(6H)-one | | | | |
| 2-(4-acetylpiperazin-1-yl)-6-isopropyl-4- | 333 | | 830 | |
| (2-methyl-1-m-tolylpropan-2-ylamino)-5H- | | | | |
| pyrrolo[3,4-d]pyrimidin-7(6H)-one | | | | |
| 2-(4-acetylpiperazin-1-yl)-4-((4-ethoxy-2- | 334 | 2968 | | |
| methoxybenzyl)(methyl)amino)-6- | | | | |
| isopropyl-5H-pyrrolo[3,4-d]pyrimidin- | | | | |
| 7(6H)-one | | | | |
| 2-(4-acetylpiperazin-1-yl)-4-(4-ethoxy-2- | 335 | 225 | | |
| methoxybenzylamino)-6-isopropyl-5H- | | | | |
| pyrrolo[3,4-d]pyrimidin-7(6H)-one | | | | |
| 2-(4-acetylpiperazin-1-yl)-4-((2-ethoxy-4- | 336 | 1607 | | |
| methoxybenzyl)(methyl)amino)-6- | | | | |
| isopropyl-5H-pyrrolo[3,4-d]pyrimidin- | | | | |
| 7(6H)-one | | | | |
| 2-(4-acetylpiperazin-1-yl)-4-(4- | 337 | 79.52 | | |
| isopropoxy-2-methoxybenzylamino)-6- | | | | |
| isopropyl-5H-pyrrolo[3,4-d]pyrimidin- | | | | |
| 7(6H)-one | | | | |
| 2-(4-acetylpiperazin-1-yl)-4-({(1R)-1-[3- | 338 | 2643 | | |
| fluoro-4- | | | | |
| (methoxymethyl)phenyl]ethyl}amino)-6- | | | | |
| (1-methylethyl)-5,6-dihydro-7H- | | | | |
| pyrrolo[3,4-d]pyrimidin-7-one | | | | |
| 2-(4-acetylpiperazin-1-yl)-4-[{[1-(4- | 339 | 2282 | | |
| fluorophenyl)-1H-pyrazol-4- | | | | |
| yl]methyl}(methyl)amino]-6-(1- | | | | |
| methylethyl)-5,6-dihydro-7H-pyrrolo[3,4- | | | | |
| d]pyrimidin-7-one | | | | |
| 2-(5,6-dihydroimidazo[1,2-a]pyrazin- | 340 | 2707 | | |
| 7(8H)-yl)-4-{[2-(4-fluorophenyl)-1,1- | | | | |
| dimethylethyl]amino}-6-(1-methylethyl)- | | | | |
| 5,6-dihydro-7H-pyrrolo[3,4-d]pyrimidin-7- | | | | |
| one | | | | |
| 2-(4-acetylpiperazin-1-yl)-4-(((7- | 341 | 29.19 | | |
| chloroisoquinolin-3- | | | | |
| yl)methyl)(methyl)amino)-6-isopropyl-5H- | | | | |
| pyrrolo[3,4-d]pyrimidin-7(6H)-one | | | | |
| 2-(4-acetylpiperazin-1-yl)-6-isopropyl-4- | 342 | 1225 | | |
| (methyl((6-methylisoquinolin-3- | | | | |
| yl)methyl)amino)-5H-pyrrolo[3,4- | | | | |
| d]pyrimidin-7(6H)-one | | | | |
| 2-(4-acetylpiperazin-1-yl)-6-isopropyl-4- | 343 | | 24 | 2967 |
| (quinolin-3-ylmethylamino)-5H- | | | | |
| pyrrolo[3,4-d]pyrimidin-7(6H)-one | | | | |
| (R)-2-(4-acetylpiperazin-1-yl)-6-isopropyl- | 344 | | 35 | 233.9 |
| 4-(1-(quinolin-3-yl)ethylamino)-5H- | | | | |
| pyrrolo[3,4-d]pyrimidin-7(6H)-one | | | | |
| (Enantiomer 1) | | | | |
| (S)-2-(4-acetylpiperazin-1-yl)-6-isopropyl- | 345 | | 66 | 1911 |
| 4-(1-(quinolin-3-yl)ethylamino)-5H- | | | | |
| pyrrolo[3,4-d]pyrimidin-7(6H)-one | | | | |
| (Enantiomer 2) | | | | |
| 2-(4-acetylpiperazin-1-yl)-6-isopropyl-4- | 346 | | 47 | 10000 |
| (methyl(quinolin-3-ylmethyl)amino)-5H- | | | | |
| pyrrolo[3,4-d]pyrimidin-7(6H)-one | | | | |
| 2-(4-acetylpiperazin-1-yl)-6-isopropyl-4- | 347 | | 2900 | |
| (methyl((2-methylquinolin-3- | | | | |
| yl)methyl)amino)-5H-pyrrolo[3,4- | | | | |
| d]pyrimidin-7(6H)-one | | | | |
| 2-(4-acetylpiperazin-1-yl)-4-(((6- | 348 | 2722 | | |
| chloroisoquinolin-3- | | | | |
| yl)methyl)(methyl)amino)-6-isopropyl-5H- | | | | |
| pyrrolo[3,4-d]pyrimidin-7(6H)-one | | | | |
| 2-(4-acetylpiperazin-1-yl)-4-(((6- | 349 | 1048 | | |
| fluoroisoquinolin-3- | | | | |
| yl)methyl)(methyl)amino)-6-isopropyl-5H- | | | | |
| pyrrolo[3,4-d]pyrimidin-7(6H)-one | | | | |
| 2-(4-acetylpiperazin-1-yl)-4-(((7- | 350 | 29.85 | | |
| fluoroisoquinolin-3- | | | | |
| yl)methyl)(methyl)amino)-6-isopropyl-5H- | | | | |
| pyrrolo[3,4-d]pyrimidin-7(6H)-one | | | | |
| (R)-4-(1-(2-(4-acetylpiperazin-1-yl)-6- | 351 | 1940 | 3300 | |
| isopropyl-7-oxo-6,7-dihydro-5H- | | | | |
| pyrrolo[3,4-d]pyrimidin-4- | | | | |
| ylamino)ethyl)benzonitrile | | | | |
| 2-(4-Acetyl-2-methylpiperazin-1-yl)-6- | 352 | | 200 | |
| isopropyl-4-(quinolin-3-ylmethylamino)- | | | | |
| 5H-pyrrolo[3,4-d]pyrimidin-7(6H)-one | | | | |
| 2-(4-acetylpiperazin-1-yl)-6-isopropyl-4- | 353 | 61.13 | | |
| (methyl((7-methylisoquinolin-3- | | | | |
| yl)methyl)amino)-5H-pyrrolo[3,4- | | | | |
| d]pyrimidin-7(6H)-one | | | | |
| 2-(4-acetylpiperazin-1-yl)-6-(1- | 354 | 494.1 | | |
| methylethyl)-4-{[(1-methyl-1H-indol-5- | | | | |
| yl)methyl]amino}-5,6-dihydro-7H- | | | | |
| pyrrolo[3,4-d]pyrimidin-7-one | | | | |
| 2-(4-acetylpiperazin-1-yl)-6-(1- | 355 | 5759 | | |
| methylethyl)-4-{methyl[(1-methyl-1H- | | | | |
| indol-6-yl)methyl]amino}-5,6-dihydro-7H- | | | | |
| pyrrolo[3,4-d]pyrimidin-7-one | | | | |
| 2-(4-acetylpiperazin-1-yl)-6-(1- | 356 | 1060 | | |
| methylethyl)-4-{[(1-methyl-1H-indol-6- | | | | |
| yl)methyl]amino}-5,6-dihydro-7H- | | | | |
| pyrrolo[3,4-d]pyrimidin-7-one | | | | |
| (R)-2-(4-acetylpiperazin-1-yl)-4-(1-(2,4- | 357 | 163.3 | | |
| diethoxyphenyl)ethylamino)-6-isopropyl- | | | | |
| 5H-pyrrolo[3,4-d]pyrimidin-7(6H)-one | | | | |
| (R)-2-(4-acetylpiperazin-1-yl)-4-(1-(4- | 358 | >30000 | | |
| ((dimethylamino)methyl)phenyl)ethylamino)- | | | | |
| 6-isopropyl-5H-pyrrolo[3,4- | | | | |
| d]pyrimidin-7(6H)-one | | | | |
| (R)-2-(4-acetylpiperazin-1-yl)-4-(1-(2,2- | 359 | 510.5 | | |
| difluorobenzo[d][1,3]dioxol-5- | | | | |
| yl)ethylamino)-6-isopropyl-5H- | | | | |
| pyrrolo[3,4-d]pyrimidin-7(6H)-one | | | | |
| 2-(4-acetylpiperazin-1-yl)-4-(((2,2- | 360 | >30000 | | |
| difluorobenzo[d][1,3]dioxol-5- | | | | |
| yl)methyl)(methyl)amino)-6-isopropyl-5H- | | | | |
| pyrrolo[3,4-d]pyrimidin-7(6H)-one | | | | |
| (R)-4-(1-(4-ethoxyphenyl)ethylamino)-2- | 361 | 326.7 | | |
| (4-ethyl-3-oxopiperazin-1-yl)-6-isopropyl- | | | | |
| 5H-pyrrolo[3,4-d]pyrimidin-7(6H)-one | | | | |
| 2-(4-ethyl-3-oxopiperazin-1-yl)-6- | 362 | 355.8 | | |
| isopropyl-4-((isoquinolin-3- | | | | |
| ylmethyl)(methyl)amino)-5H-pyrrolo[3,4- | | | | |
| d]pyrimidin-7(6H)-one | | | | |
| (R)-4-(1-(4-ethoxyphenyl)ethylamino)-6- | 363 | 574.1 | | |
| isopropyl-2-(3-oxo-4-(2,2,2- | | | | |
| trifluoroethyl)piperazin-1-yl)-5H- | | | | |
| pyrrolo[3,4-d]pyrimidin-7(6H)-one | | | | |
| 6-isopropyl-4-((isoquinolin-3- | 364 | 795.1 | | |
| ylmethyl)(methyl)amino)-2-(3-oxo-4- | | | | |
| (2,2,2-trifluoroethyl)piperazin-1-yl)-5H- | | | | |
| pyrrolo[3,4-d]pyrimidin-7(6H)-one | | | | |

The following compounds have also been tested and are found to have IC₅₀ greater than 3300 nM.

These compounds having IC₅₀ greater than 3300 nM, which are less preferred, are:
2-(4-acetylpiperazin-1-yl)-4-[{[1-(3-methoxyphenyl)-1H-pyrazol-4-yl]methyl}(methyl)amino]-6-(1-methylethyl)-5,6-dihydro-7H-pyrrolo[3,4-d]pyrimidin-7-one;
(R)-2-(4-acetylpiperazin-1-yl)-6-isopropyl-4-(1-(4-(methoxymethyl)phenyl)ethylamino)-5H-pyrrolo[3,4-d]pyrimidin-7(6H)-one;
2-(4-acetylpiperazin-1-yl)-6-(1-methylethyl)-4-(methyl {[1-(3-methylphenyl)-1H-pyrazol-4-yl]methyl}amino)-5,6-dihydro-7H-pyrrolo[3,4-d]pyrimidin-7-one;
2-(4-acetylpiperazin-1-yl)-4-{2-[3-fluoro-4-(2-hydroxyethoxy)phenyl]pyrrolidin-1-yl}-6-(1-methylethyl)-5,6-dihydro-7H-pyrrolo[3,4-d]pyrimidin-7-one;
N-[2-(4-acetylpiperazin-1-yl)-6-(1-methylethyl)-7-oxo-6,7-dihydro-5H-pyrrolo[3,4-d]pyrimidin-4-yl]-4-ethoxybenzamide;
4-({1-[2-(4-acetylpiperazin-1-yl)-6-(1-methylethyl)-7-oxo-6,7-dihydro-5H-pyrrolo[3,4-d]pyrimidin-4-yl]pyrrolidin-2-yl}methyl)benzonitrile;
2-(4-acetylpiperazin-1-yl)-4-{[1-(2,3-dihydro-1,4-benzodioxin-2-yl)ethyl]amino}-6-(1-methylethyl)-5,6-dihydro-7H-pyrrolo[3,4-d]pyrimidin-7-one;
2-(4-acetylpiperazin-1-yl)-4-[(2,3-dihydro-1-benzofuran-2-ylmethyl)(methyl)amino]-6-(1-methylethyl)-5,6-dihydro-7H-pyrrolo[3,4-d]pyrimidin-7-one;
2-(4-acetylpiperazin-1-yl)-4-{[(6-methoxy-3,4-dihydro-2H-chromen-3-yl)methyl]amino}-6-(1-methylethyl)-5,6-dihydro-7H-pyrrolo[3,4-d]pyrimidin-7-one;
2-(4-acetylpiperazin-1-yl)-4-[(2,3-dihydro-1H-inden-2-ylmethyl)amino]-6-(1-methylethyl)-5,6-dihydro-7H-pyrrolo[3,4-d]pyrimidin-7-one;
2-(4-acetylpiperazin-1-yl)-4-{[(1S)-1-(4-ethoxy-3-methylphenyl)ethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-[2-(quinolin-3-ylmethyl)pyrrolidin-1-yl]-5,6-dihydro-7H-pyrrolo[3,4-d]pyrimidin-7-one;
2-(4-acetylpiperazin-1-yl)-4-[(5-chloro-2,3-dihydro-1-benzofuran-3-yl)amino]-6-(1-methylethyl)-5,6-dihydro-7H-pyrrolo[3,4-d]pyrimidin-7-one;
2-(4-acetylpiperazin-1-yl)-4-[(6-methoxy-1,2,3,4-tetrahydronaphthalen-1-yl)amino]-6-(1-methylethyl)-5,6-dihydro-7H-pyrrolo[3,4-d]pyrimidin-7-one;
6-(1-methylethyl)-2-[(2R)-2-(pyrrolidin-1-ylmethyl)pyrrolidin-1-yl]-4-[(quinolin-3-ylmethyl)amino]-5,6-dihydro-7H-pyrrolo[3,4-d]pyrimidin-7-one;
6-(1-methylethyl)-2-[4-(2-morpholin-4-yl-2-oxoethyl)piperazin-1-yl]-4-[(quinolin-3-ylmethyl)amino]-5,6-dihydro-7H-pyrrolo[3,4-d]pyrimidin-7-one;
6-(1-methylethyl)-2-[(2S)-2-(pyrrolidin-1-ylmethyl)pyrrolidin-1-yl]-4-[(quinolin-3-ylmethyl)amino]-5,6-dihydro-7H-pyrrolo[3,4-d]pyrimidin-7-one;
2-{[2-(dimethylamino)-2-phenylethyl]amino}-6-(1-methylethyl)-4-[(quinolin-3-ylmethyl)amino]-5,6-dihydro-7H-pyrrolo[3,4-d]pyrimidin-7-one;
2-{[2-(dimethylamino)-1-phenylethyl] (methyl)amino}-6-(1-methylethyl)-4-[(quinolin-3-ylmethyl)amino]-5,6-dihydro-7H-pyrrolo[3,4-d]pyrimidin-7-one;
2-{2-[(dimethylamino)methyl]piperidin-1-yl}-6-(1-methylethyl)-4-[(quinolin-3-ylmethyl)amino]-5,6-dihydro-7H-pyrrolo[3,4-d]pyrimidin-7-one;
2-[3-(diethylamino)pyrrolidin-1-yl]-6-(1-methylethyl)-4-[(quinolin-3-ylmethyl)amino]-5,6-dihydro-7H-pyrrolo[3,4-d]pyrimidin-7-one;
6-(1-methylethyl)-4-[(quinolin-3-ylmethyl)amino]-2-[(tetrahydro-2H-pyran-4-ylmethyl)amino]-5,6-dihydro-7H-pyrrolo[3,4-d]pyrimidin-7-one;
6-(1-methylethyl)-2-[methyl(tetrahydro-2H-pyran-4-ylmethyl)amino]-4-[(quinolin-3-ylmethyl)amino]-5,6-dihydro-7H-pyrrolo[3,4-d]pyrimidin-7-one;
2-{[(1,1-dioxidotetrahydrothiophen-3-yl)methyl]amino}-6-(1-methylethyl)-4-[(quinolin-3-ylmethyl)amino]-5,6-dihydro-7H-pyrrolo[3,4-d]pyrimidin-7-one;
6-(1-methylethyl)-4-[(quinolin-3-ylmethyl)amino]-2-(tetrahydro-2H-pyran-4-ylamino)-5,6-dihydro-7H-pyrrolo[3,4-d]pyrimidin-7-one;
2-[(2,2-difluoroethyl)amino]-6-(1-methylethyl)-4-[(quinolin-3-ylmethyl)amino]-5,6-dihydro-7H-pyrrolo[3,4-d]pyrimidin-7-one;
6-(1-methylethyl)-2-{[(1-methyl-1H-imidazol-4-yl)methyl]amino}-4-[(quinolin-3-ylmethyl)amino]-5,6-dihydro-7H-pyrrolo[3,4-d]pyrimidin-7-one;
2-[(1,1-dioxidotetrahydrothiophen-3-yl)amino]-6-(1-methylethyl)-4-[(quinolin-3-ylmethyl)amino]-5,6-dihydro-7H-pyrrolo[3,4-d]pyrimidin-7-one;
N,N-dimethyl-1-{6-(1-methylethyl)-7-oxo-4-[(quinolin-3-ylmethyl)amino]-6,7-dihydro-5H-pyrrolo[3,4-d]pyrimidin-2-yl}-L-prolinamide;
ethyl 4-{6-(1-methylethyl)-7-oxo-4-[(quinolin-3-ylmethyl)amino]-6,7-dihydro-5H-pyrrolo[3,4-d]pyrimidin-2-yl}piperazine-1-carboxylate;
2-(4-methyl-1,4-diazepan-1-yl)-6-(1-methylethyl)-4-[(quinolin-3-ylmethyl)amino]-5,6-dihydro-7H-pyrrolo[3,4-d]pyrimidin-7-one;
N,N-dimethyl-2-(4-{6-(1-methylethyl)-7-oxo-4-[(quinolin-3-ylmethyl)amino]-6,7-dihydro-5H-pyrrolo[3,4-d]pyrimidin-2-yl}piperazin-1-yl)acetamide;
3-(4-{6-(1-methylethyl)-7-oxo-4-[(quinolin-3-ylmethyl)amino]-6,7-dihydro-5H-pyrrolo[3,4-d]pyrimidin-2-yl}piperazin-1-yl)propanenitrile;
tert-butyl (2R)-4-{4-[(isoquinolin-3-ylmethyl)amino]-6-(1-methylethyl)-7-oxo-6,7-dihydro-5H-pyrrolo[3,4-d]pyrimidin-2-yl}-2-methylpiperazine-1-carboxylate;
tert-butyl (2S)-4-{4-[(isoquinolin-3-ylmethyl)amino]-6-(1-methylethyl)-7-oxo-6,7-dihydro-5H-pyrrolo[3,4-d]pyrimidin-2-yl}-2-methylpiperazine-1-carboxylate;
tert-butyl (2R)-2-methyl-4-{6-(1-methylethyl)-7-oxo-4-[(quinolin-3-ylmethyl)amino]-6,7-dihydro-5H-pyrrolo[3,4-d]pyrimidin-2-yl}piperazine-1-carboxylate;
tert-butyl (2S)-2-methyl-4-{6-(1-methylethyl)-7-oxo-4-[(quinolin-3-ylmethyl)amino]-6,7-dihydro-5H-pyrrolo[3,4-d]pyrimidin-2-yl}piperazine-1-carboxylate;
3-[(1R)-1-{[2-(4-acetylpiperazin-1-yl)-6-(1-methylethyl)-7-oxo-6,7-dihydro-5H-pyrrolo[3,4-d]pyrimidin-4-yl]amino}ethyl]benzonitrile;
2-(4-acetylpiperazin-1-yl)-6-(1-methylethyl)-4-{[(5-methyl-3-phenylisoxazol-4-yl)methyl]amino}-5,6-dihydro-7H-pyrrolo[3,4-d]pyrimidin-7-one;
2-(4-acetylpiperazin-1-yl)-6-(1-methylethyl)-4-({[1-(1-methylethyl)-1H-pyrazol-4-yl]methyl}amino)-5,6-dihydro-7H-pyrrolo[3,4-d]pyrimidin-7-one;
2-(4-acetylpiperazin-1-yl)-6-(1-methylethyl)-4-{2-[3-(1-methylethyl)-1,2,4-oxadiazol-5-yl]pyrrolidin-1-yl}-5,6-dihydro-7H-pyrrolo[3,4-d]pyrimidin-7-one;
2-(4-acetylpiperazin-1-yl)-6-(1-methylethyl)-4-({1-[3-(1-methylethyl)-1,2,4-oxadiazol-5-yl]ethyl}amino)-5,6-dihydro-7H-pyrrolo[3,4-d]pyrimidin-7-one;
N,N-dimethyl-1-{6-(1-methylethyl)-7-oxo-4-[(quinolin-3-ylmethyl)amino]-6,7-dihydro-5H-pyrrolo[3,4-d]pyrimidin-2-yl}piperidine-4-carboxamide;
6-(1-methylethyl)-2-{[(5-oxopyrrolidin-2-yl)methyl]amino}-4-[(quinolin-3-ylmethyl)amino]-5,6-dihydro-7H-pyrrolo[3,4-d]pyrimidin-7-one;
2-(4-acetylpiperazin-1-yl)-6-(1-methylethyl)-4-[(quinolin-4-ylmethyl)amino]-5,6-dihydro-7H-pyrrolo[3,4-d]pyrimidin-7-one;
1-{6-(1-methylethyl)-7-oxo-4-[(quinolin-3-ylmethyl)amino]-6,7-dihydro-5H-pyrrolo[3,4-d]pyrimidin-2-yl}piperidine-4-carboxylic acid;
N-methyl-1-{6-(1-methylethyl)-7-oxo-4-[(quinolin-3-ylmethyl)amino]-6,7-dihydro-5H-pyrrolo[3,4-d]pyrimidin-2-yl}piperidine-4-carboxamide;
6-(1-methylethyl)-2-{[(3S)-6-oxopiperidin-3-yl]amino}-4-[(quinolin-3-ylmethyl)amino]-5,6-dihydro-7H-pyrrolo[3,4-d]pyrimidin-7-one;
2-(4-acetylpiperazin-1-yl)-4-{[(5-cyclopropyl-1H-pyrazol-3-yl)methyl]amino}-6-(1-methylethyl)-5,6-dihydro-7H-pyrrolo[3,4-d]pyrimidin-7-one;
2-[4-(2-methoxyethyl)piperazin-1-yl]-6-(1-methylethyl)-4-[(quinolin-3-ylmethyl)amino]-5,6-dihydro-7H-pyrrolo[3,4-d]pyrimidin-7-one;
2-[(3R)-3-(hydroxymethyl)piperazin-1-yl]-6-(1-methylethyl)-4-[(quinolin-3-ylmethyl)amino]-5,6-dihydro-7H-pyrrolo[3,4-d]pyrimidin-7-one;
2-[4-(2-hydroxyethyl)piperazin-1-yl]-6-(1-methylethyl)-4-[(quinolin-3-ylmethyl)amino]-5,6-dihydro-7H-pyrrolo[3,4-d]pyrimidin-7-one;
N-[2-({6-(1-methylethyl)-7-oxo-4-[(quinolin-3-ylmethyl)amino]-6,7-dihydro-5H-pyrrolo[3,4-d]pyrimidin-2-yl}amino)ethyl]acetamide;
6-(1-methylethyl)-2-{[2-(2-oxopyrrolidin-1-yl)ethyl]amino}-4-[(quinolin-3-ylmethyl)amino]-5,6-dihydro-7H-pyrrolo[3,4-d]pyrimidin-7-one;
2-(4-acetylpiperazin-1-yl)-6-(1-methylethyl)-4-{[3-(1H-pyrazol-1-yl)benzyl]amino}-5,6-dihydro-7H-pyrrolo[3,4-d]pyrimidin-7-one;
1-[2-(4-acetylpiperazin-1-yl)-6-(1-methylethyl)-7-oxo-6,7-dihydro-5H-pyrrolo[3,4-d]pyrimidin-4-yl]-N-cyclohexyl-N-methyl-D-prolinamide;
2-(4-acetylpiperazin-1-yl)-4-(2-cyclohexylpyrrolidin-1-yl)-6-(1-methylethyl)-5,6-dihydro-7H-pyrrolo[3,4-d]pyrimidin-7-one;
2-(4-acetylpiperazin-1-yl)-6-(1-methylethyl)-4-{[1-(3-pyridin-3-yl-1,2,4-oxadiazol-5-yl)ethyl]amino}-5,6-dihydro-7H-pyrrolo[3,4-d]pyrimidin-7-one;
2-(4-acetylpiperazin-1-yl)-4-({1-[3-(2-methoxyethyl)-1,2,4-oxadiazol-5-yl]ethyl}amino)-6-(1-methylethyl)-5,6-dihydro-7H-pyrrolo[3,4-d]pyrimidin-7-one;
N-(2-{[6-(1-methylethyl)-7-oxo-4-{[(3-phenyl-1,2,4-oxadiazol-5-yl)methyl]amino}-6,7-dihydro-5H-pyrrolo[3,4-d]pyrimidin-2-yl]amino}ethyl)acetamide;
6-(1-methylethyl)-2-{[2-(2-oxopyrrolidin-1-yl)ethyl]amino}-4-{[(3-phenyl-1,2,4-oxadiazol-5-yl)methyl]amino}-5,6-dihydro-7H-pyrrolo[3,4-d]pyrimidin-7-one;
1-[2-(4-acetylpiperazin-1-yl)-6-(1-methylethyl)-7-oxo-6,7-dihydro-5H-pyrrolo[3,4-d]pyrimidin-4-yl]-N-benzyl-N-methyl-D-prolinamide;
2-(4-acetylpiperazin-1-yl)-6-(1-methylethyl)-4-(2-pyridin-3-ylpyrrolidin-1-yl)-5,6-dihydro-7H-pyrrolo[3,4-d]pyrimidin-7-one;
2-(4-acetylpiperazin-1-yl)-6-(1-methylethyl)-4-(2-pyridin-4-ylpyrrolidin-1-yl)-5,6-dihydro-7H-pyrrolo[3,4-d]pyrimidin-7-one;
2-[(2R,6S)-2,6-dimethylmorpholin-4-yl]-6-(1-methylethyl)-4-{[(3-phenyl-1,2,4-oxadiazol-5-yl)methyl]amino}-5,6-dihydro-7H-pyrrolo[3,4-d]pyrimidin-7-one;
N-{1-[6-(1-methylethyl)-7-oxo-4-{[(3-phenyl-1,2,4-oxadiazol-5-yl)methyl]amino}-6,7-dihydro-5H-pyrrolo[3,4-d]pyrimidin-2-yl]pyrrolidin-3-yl}acetamide;
N-methyl-N-{1-[6-(1-methylethyl)-7-oxo-4-{[(3-phenyl-1,2,4-oxadiazol-5-yl)methyl]amino}-6,7-dihydro-5H-pyrrolo[3,4-d]pyrimidin-2-yl]pyrrolidin-3-yl}acetamide;
6-(1-methylethyl)-2-(4-methyl-3-oxopiperazin-1-yl)-4-{[(3-phenyl-1,2,4-oxadiazol-5-yl)methyl]amino}-5,6-dihydro-7H-pyrrolo[3,4-d]pyrimidin-7-one;
2-(4-acetylpiperazin-1-yl)-4-[(isoquinolin-1-ylmethyl)amino]-6-(1-methylethyl)-5,6-dihydro-7H-pyrrolo[3,4-d]pyrimidin-7-one;
2-(4-acetylpiperazin-1-yl)-4-{[(1R,2S)-2-hydroxy-2,3-dihydro-1H-inden-1-yl]amino}-6-(1-methylethyl)-5,6-dihydro-7H-pyrrolo[3,4-d]pyrimidin-7-one;
3-chlorobenzyl 1-[2-(4-acetylpiperazin-1-yl)-6-(1-methylethyl)-7-oxo-6,7-dihydro-5H-pyrrolo[3,4-d]pyrimidin-4-yl]-D-prolinate;
2-(4-acetylpiperazin-1-yl)-6-(1-methylethyl)-4-{[(5-methylpyridin-2-yl)methyl]amino}-5,6-dihydro-7H-pyrrolo[3,4-d]pyrimidin-7-one;
2-(4-acetylpiperazin-1-yl)-4-[(6-chloro-2,3-dihydro-1H-inden-1-yl)amino]-6-(1-methylethyl)-5,6-dihydro-7H-pyrrolo[3,4-d]pyrimidin-7-one;
2-(4-acetylpiperazin-1-yl)-4-(2,3-dihydro-1H-inden-1-ylamino)-6-(1-methylethyl)-5,6-dihydro-7H-pyrrolo[3,4-d]pyrimidin-7-one;
4-{[2-(4-fluorophenyl)-1,1-dimethylethyl]amino}-2-[(3R)-3-(hydroxymethyl)piperazin-1-yl]-6-(1-methylethyl)-5,6-dihydro-7H-pyrrolo[3,4-d]pyrimidin-7-one;
2-(4-acetylpiperazin-1-yl)-6-(1-methylethyl)-4-{[(2-phenyl-1,3-thiazol-4-yl)methyl]amino}-5,6-dihydro-7H-pyrrolo[3,4-d]pyrimidin-7-one;
2-(dimethylamino)-6-(1-methylethyl)-4-{[(3-phenyl-1,2,4-oxadiazol-5-yl)methyl]amino}-5,6-dihydro-7H-pyrrolo[3,4-d]pyrimidin-7-one;
benzyl 1-[2-(4-acetylpiperazin-1-yl)-6-(1-methylethyl)-7-oxo-6,7-dihydro-5H-pyrrolo[3,4-d]pyrimidin-4-yl]-L-prolinate;
2-(4-acetylpiperazin-1-yl)-4-[(4-fluoro-2,3-dihydro-1H-inden-1-yl)amino]-6-(1-methylethyl)-5,6-dihydro-7H-pyrrolo[3,4-d]pyrimidin-7-one;
1-[2-(4-acetylpiperazin-1-yl)-6-(1-methylethyl)-7-oxo-6,7-dihydro-5H-pyrrolo[3,4-d]pyrimidin-4-yl]-N-benzyl-D-prolinamide;
2-(4-acetylpiperazin-1-yl)-4-[3-(benzyloxy)pyrrolidin-1-yl]-6-(1-methylethyl)-5,6-dihydro-7H-pyrrolo[3,4-d]pyrimidin-7-one;
2-(4-acetylpiperazin-1-yl)-6-(1-methylethyl)-4-{methyl[(5-methyl-1H-pyrazol-3-yl)methyl]amino}-5,6-dihydro-7H-pyrrolo[3,4-d]pyrimidin-7-one;
2-(4-acetylpiperazin-1-yl)-6-(1-methylethyl)-4-({[3-(2-methylpropyl)-1,2,4-oxadiazol-5-yl]methyl}amino)-5,6-dihydro-7H-pyrrolo[3,4-d]pyrimidin-7-one;
2-(4-acetylpiperazin-1-yl)-4-[(1H-indol-2-ylmethyl)amino]-6-(1-methylethyl)-5,6-dihydro-7H-pyrrolo[3,4-d]pyrimidin-7-one;
2-(4-acetylpiperazin-1-yl)-6-(1-methylethyl)-4-[(quinoxalin-6-ylmethyl)amino]-5,6-dihydro-7H-pyrrolo[3,4-d]pyrimidin-7-one;
2-(4-acetylpiperazin-1-yl)-4-[(1H-indol-3-ylmethyl)amino]-6-(1-methylethyl)-5,6-dihydro-7H-pyrrolo[3,4-d]pyrimidin-7-one;
2-{[2-(dimethylamino)ethyl] (methyl)amino}-6-(1-methylethyl)-4-[(quinolin-3-ylmethyl)amino]-5,6-dihydro-7H-pyrrolo[3,4-d]pyrimidin-7-one;
2-(4-acetylpiperazin-1-yl)-4-{[(1S,2S)-2-hydroxy-2,3-dihydro-1H-inden-1-yl]amino}-6-(1-methylethyl)-5,6-dihydro-7H-pyrrolo[3,4-d]pyrimidin-7-one;
4-{[2-(4-fluorophenyl)-1,1-dimethylethyl]amino}-2-[(3S)-3-(hydroxymethyl)piperazin-1-yl]-6-(1-methylethyl)-5,6-dihydro-7H-pyrrolo[3,4-d]pyrimidin-7-one;
N-({(2R)-1-[2-(4-acetylpiperazin-1-yl)-6-(1-methylethyl)-7-oxo-6,7-dihydro-5H-pyrrolo[3,4-d]pyrimidin-4-yl]pyrrolidin-2-yl}methyl)benzamide;
1-[2-(4-acetylpiperazin-1-yl)-6-(1-methylethyl)-7-oxo-6,7-dihydro-5H-pyrrolo[3,4-d]pyrimidin-4-yl]-N-benzyl-N-ethyl-D-prolinamide;
N-[2-(4-acetylpiperazin-1-yl)-6-(1-methylethyl)-7-oxo-6,7-dihydro-5H-pyrrolo[3,4-d]pyrimidin-4-yl]-2-(4-fluorophenyl)acetamide;
2-{[2-(dimethylamino)ethyl]amino}-6-(1-methylethyl)-4-[(quinolin-3-ylmethyl)amino]-5,6-dihydro-7H-pyrrolo[3,4-d]pyrimidin-7-one;
2-(4-acetylpiperazin-1-yl)-6-(1-methylethyl)-4-{[(1,3,5-trimethyl-1H-pyrazol-4-yl)methyl]amino}-5,6-dihydro-7H-pyrrolo[3,4-d]pyrimidin-7-one;
2-(4-acetylpiperazin-1-yl)-4-{[(1,5-dimethyl-1H-pyrazol-3-yl)methyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-{[(1-methyl-1H-pyrazol-3-yl)methyl]amino}-5,6-dihydro-7H-pyrrolo[3,4-d]pyrimidin-7-one;
2-[(2-hydroxyethyl)amino]-6-(1-methylethyl)-4-[(quinolin-3-ylmethyl)amino]-5,6-dihydro-7H-pyrrolo[3,4-d]pyrimidin-7-one;
2-[(2-methoxyethyl)amino]-6-(1-methylethyl)-4-[(quinolin-3-ylmethyl)amino]-5,6-dihydro-7H-pyrrolo[3,4-d]pyrimidin-7-one;
2-(4-acetylpiperazin-1-yl)-4-[2-(ethoxymethyl)pyrrolidin-1-yl]-6-(1-methylethyl)-5,6-dihydro-7H-pyrrolo[3,4-d]pyrimidin-7-one;
2-(ethylamino)-6-(1-methylethyl)-4-[(quinolin-3-ylmethyl)amino]-5,6-dihydro-7H-pyrrolo[3,4-d]pyrimidin-7-one;
2-(4-acetylpiperazin-1-yl)-6-(1-methylethyl)-4-[(pyrazin-2-ylmethyl)amino]-5,6-dihydro-7H-pyrrolo[3,4-d]pyrimidin-7-one;
2-(4-acetylpiperazin-1-yl)-6-(1-methylethyl)-4-{[(2-pyrrolidin-1-ylpyridin-3-yl)methyl]amino}-5,6-dihydro-7H-pyrrolo[3,4-d]pyrimidin-7-one;
2-(4-acetylpiperazin-1-yl)-6-(1-methylethyl)-4-{[(1-methyl-1H-imidazol-4-yl)methyl]amino}-5,6-dihydro-7H-pyrrolo[3,4-d]pyrimidin-7-one;
4-{[2-(4-fluorophenyl)-1,1-dimethylethyl]amino}-2-[(8aS)-hexahydropyrrolo[1,2-a]pyrazin-2(1H)-yl]-6-(1-methylethyl)-5,6-dihydro-7H-pyrrolo[3,4-d]pyrimidin-7-one;
4-{[2-(4-fluorophenyl)-1,1-dimethylethyl]amino}-2-[4-(2-hydroxyethyl)piperazin-1-yl]-6-(1-methylethyl)-5,6-dihydro-7H-pyrrolo[3,4-d]pyrimidin-7-one;
4-{[2-(4-fluorophenyl)-1,1-dimethylethyl]amino}-2-[4-(2-methoxyethyl)piperazin-1-yl]-6-(1-methylethyl)-5,6-dihydro-7H-pyrrolo[3,4-d]pyrimidin-7-one;
2-(4-acetylpiperazin-1-yl)-6-(1-methylethyl)-4-{[(3-methyl-1H-pyrazol-5-yl)methyl]amino}-5,6-dihydro-7H-pyrrolo[3,4-d]pyrimidin-7-one;
2-(4-acetylpiperazin-1-yl)-6-(1-methylethyl)-4-{[(5-methylisoxazol-3-yl)methyl]amino}-5,6-dihydro-7H-pyrrolo[3,4-d]pyrimidin-7-one;
2-(4-acetylpiperazin-1-yl)-4-{[(1,3-dimethyl-1H-pyrazol-5-yl)methyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-({[5-(trifluoromethyl)furan-2-yl]methyl}amino)-5,6-dihydro-7H-pyrrolo[3,4-d]pyrimidin-7-one;
2-(4-acetylpiperazin-1-yl)-4-{[(1R)-1-(3-fluorophenyl)ethyl]amino}-6-(1-methylethyl)-5,6-dihydro-7H-pyrrolo[3,4-d]pyrimidin-7-one;
2-(4-acetylpiperazin-1-yl)-4-[(6-fluoro-2,3-dihydro-1H-inden-1-yl)amino]-6-(1-methylethyl)-5,6-dihydro-7H-pyrrolo[3,4-d]pyrimidin-7-one;
4-{[2-(4-fluorophenyl)-1,1-dimethylethyl]amino}-6-(1-methylethyl)-2-[4-(1-methylethyl)piperazin-1-yl]-5,6-dihydro-7H-pyrrolo[3,4-d]pyrimidin-7-one;
2-(4-acetylpiperazin-1-yl)-4-[(5-methoxy-2,3-dihydro-1H-inden-1-yl)amino]-6-(1-methylethyl)-5,6-dihydro-7H-pyrrolo[3,4-d]pyrimidin-7-one;
2-(4-acetylpiperazin-1-yl)-4-[(5-fluoro-2,3-dihydro-1H-inden-1-yl)amino]-6-(1-methylethyl)-5,6-dihydro-7H-pyrrolo[3,4-d]pyrimidin-7-one;
2-(4-acetylpiperazin-1-yl)-4-{[(1R,2R)-2-hydroxy-2,3-dihydro-1H-inden-1-yl]amino}-6-(1-methylethyl)-5,6-dihydro-7H-pyrrolo[3,4-d]pyrimidin-7-one;
2-(4-acetylpiperazin-1-yl)-6-(1-methylethyl)-4-[2-(2-methylpropyl)pyrrolidin-1-yl]-5,6-dihydro-7H-pyrrolo[3,4-d]pyrimidin-7-one;
2-(4-acetylpiperazin-1-yl)-4-[(2R)-2-(methoxymethyl)pyrrolidin-1-yl]-6-(1-methylethyl)-5,6-dihydro-7H-pyrrolo[3,4-d]pyrimidin-7-one;
2-(4-acetylpiperazin-1-yl)-6-(1-methylethyl)-4-[(2R)-2-(pyrrolidin-1-ylmethyl)pyrrolidin-1-yl]-5,6-dihydro-7H-pyrrolo[3,4-d]pyrimidin-7-one;
2-(4-acetylpiperazin-1-yl)-6-(1-methylethyl)-4-[2-(morpholin-4-ylmethyl)pyrrolidin-1-yl]-5,6-dihydro-7H-pyrrolo[3,4-d]pyrimidin-7-one;
2-(4-acetylpiperazin-1-yl)-4-{[(1S,2R)-2-hydroxy-2,3-dihydro-1H-inden-1-yl]amino}-6-(1-methylethyl)-5,6-dihydro-7H-pyrrolo[3,4-d]pyrimidin-7-one;
2-(4-acetylpiperazin-1-yl)-6-(1-methylethyl)-4-({[2-methyl-6-(trifluoromethyl)pyridin-3-yl]methyl}amino)-5,6-dihydro-7H-pyrrolo[3,4-d]pyrimidin-7-one;
2-(4-acetylpiperazin-1-yl)-6-(1-methylethyl)-4-{2-[(3-methylpyridin-2-yl)methyl]pyrrolidin-1-yl}-5,6-dihydro-7H-pyrrolo[3,4-d]pyrimidin-7-one;
2-(4-acetylpiperazin-1-yl)-6-(1-methylethyl)-4-{2-[(6-methylpyridin-2-yl)methyl]pyrrolidin-1-yl}-5,6-dihydro-7H-pyrrolo[3,4-d]pyrimidin-7-one;
2-(4-acetylpiperazin-1-yl)-6-(1-methylethyl)-4-[2-(pyridin-4-ylmethyl)piperidin-1-yl]-5,6-dihydro-7H-pyrrolo[3,4-d]pyrimidin-7-one;
2-(4-acetylpiperazin-1-yl)-6-(1-methylethyl)-4-[2-(pyridin-2-ylmethyl)pyrrolidin-1-yl]-5,6-dihydro-7H-pyrrolo[3,4-d]pyrimidin-7-one;
(4R)-4-{[2-(4-acetylpiperazin-1-yl)-6-(1-methylethyl)-7-oxo-6,7-dihydro-5H-pyrrolo[3,4-d]pyrimidin-4-yl]amino}-5-phenylpentanoic acid;
ethyl 4-{[2-(4-acetylpiperazin-1-yl)-6-(1-methylethyl)-7-oxo-6,7-dihydro-5H-pyrrolo[3,4-d]pyrimidin-4-yl]amino}butanoate;
2-(4-acetylpiperazin-1-yl)-4-amino-6-(1-methylethyl)-5,6-dihydro-7H-pyrrolo[3,4-d]pyrimidin-7-one;
2-(4-acetylpiperazin-1-yl)-6-(1-methylethyl)-4-({[6-(trifluoromethyl)pyridin-3-yl]methyl}amino)-5,6-dihydro-7H-pyrrolo[3,4-d]pyrimidin-7-one;
2-(4-acetylpiperazin-1-yl)-4-[2-(2-methoxyphenyl)pyrrolidin-1-yl]-6-(1-methylethyl)-5,6-dihydro-7H-pyrrolo[3,4-d]pyrimidin-7-one;
2-(4-acetylpiperazin-1-yl)-6-(1-methylethyl)-4-(2-pyridin-2-ylpyrrolidin-1-yl)-5,6-dihydro-7H-pyrrolo[3,4-d]pyrimidin-7-one;
2-(4-acetylpiperazin-1-yl)-6-(1-methylethyl)-4-{[(5-methylpyrazin-2-yl)methyl]amino}-5,6-dihydro-7H-pyrrolo[3,4-d]pyrimidin-7-one;
2-(4-acetylpiperazin-1-yl)-4-(3-benzylpyrrolidin-1-yl)-6-(1-methylethyl)-5,6-dihydro-7H-pyrrolo[3,4-d]pyrimidin-7-one;
2-(4-acetylpiperazin-1-yl)-4-{[(1R)-1-(2-fluorophenyl)ethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-[2-(pyrrolidin-1-ylcarbonyl)pyrrolidin-1-yl]-5,6-dihydro-7H-pyrrolo[3,4-d]pyrimidin-7-one;
2-(4-acetylpiperazin-1-yl)-4-{[1-(4-fluorophenyl)-2-methoxyethyl]amino}-6-(1-methylethyl)-5,6-dihydro-7H-pyrrolo[3,4-d]pyrimidin-7-one;
2-(4-acetylpiperazin-1-yl)-4-{[1-(4-fluorophenyl)-2-hydroxyethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-({(1S)-1-[4-(trifluoromethoxy)phenyl]ethyl}amino)-5,6-dihydro-7H-pyrrolo[3,4-d]pyrimidin-7-one;
4-{[2-(4-chlorophenyl)-1,1-dimethylethyl]amino}-2-(diethylamino)-6-(1-methylethyl)-5,6-dihydro-7H-pyrrolo[3,4-d]pyrimidin-7-one;
ethyl 4-[4-{[2-(4-chlorophenyl)-1,1-dimethylethyl]amino}-6-(1-methylethyl)-7-oxo-6,7-dihydro-5H-pyrrolo[3,4-d]pyrimidin-2-yl]piperazine-1-carboxylate;
4-[(biphenyl-4-ylmethyl)amino]-2-{[2-(dimethylamino)ethyl]amino}-6-(1-methylethyl)-5,6-dihydro-7H-pyrrolo[3,4-d]pyrimidin-7-one;
4-(4-acetylpiperazin-1-yl)-2-[2-(4-chlorobenzyl)pyrrolidin-1-yl]-6-(1-methylethyl)-5,6-dihydro-7H-pyrrolo[3,4-d]pyrimidin-7-one;
4-(4-acetylpiperazin-1-yl)-2-{[2-(4-chlorophenyl)propyl]amino}-6-(1-methylethyl)-5,6-dihydro-7H-pyrrolo[3,4-d]pyrimidin-7-one;
2-{[2-(dimethylamino)ethyl]amino}-4-{[1-(4-fluorophenyl)cyclopropyl]amino}-6-(1-methylethyl)-5,6-dihydro-7H-pyrrolo[3,4-d]pyrimidin-7-one;
4-{[(4-chlorophenyl)(1-methyl-1H-imidazol-2-yl)methyl]amino}-2-{[2-(dimethylamino)ethyl]amino}-6-(1-methylethyl)-5,6-dihydro-7H-pyrrolo[3,4-d]pyrimidin-7-one;
4-(4-acetylpiperazin-1-yl)-2-{[1-(4-methoxyphenyl)-1-methylethyl]amino}-6-(1-methylethyl)-5,6-dihydro-7H-pyrrolo[3,4-d]pyrimidin-7-one;
2-(4-acetylpiperazin-1-yl)-4-{[1-(4-fluorophenyl)cyclopropyl]amino}-6-(1-methylethyl)-5,6-dihydro-7H-pyrrolo[3,4-d]pyrimidin-7-one;
4-{[(4-chlorophenyl)(pyridin-4-yl)methyl]amino}-2-{[2-(dimethylamino)ethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-{[1-(4-morpholin-4-ylphenyl)ethyl]amino}-5,6-dihydro-7H-pyrrolo[3,4-d]pyrimidin-7-one;
3-{[2-(4-acetylpiperazin-1-yl)-6-(1-methylethyl)-7-oxo-6,7-dihydro-5H-pyrrolo[3,4-d]pyrimidin-4-yl]amino}-3-[4-(1-methylethoxy)phenyl]propanoic acid;
2-(4-acetylpiperazin-1-yl)-4-{[1-(4-methoxyphenyl)-1-methylethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-({1-[6-(2,2,2-trifluoroethoxy)pyridin-3-yl]ethyl}amino)-5,6-dihydro-7H-pyrrolo[3,4-d]pyrimidin-7-one;
2-(4-acetylpiperazin-1-yl)-6-(1-methylethyl)-4-[(1-pyridin-4-ylethyl)amino]-5,6-dihydro-7H-pyrrolo[3,4-d]pyrimidin-7-one;
2-(4-acetylpiperazin-1-yl)-4-[(2-methoxy-1-methylethyl)amino]-6-(1-methylethyl)-5,6-dihydro-7H-pyrrolo[3,4-d]pyrimidin-7-one;
2-(4-acetylpiperazin-1-yl)-4-[(1-cyclohexylethyl)amino]-6-(1-methylethyl)-5,6-dihydro-7H-pyrrolo[3,4-d]pyrimidin-7-one;
2,4-bis(4-acetylpiperazin-1-yl)-6-(1-methylethyl)-5,6-dihydro-7H-pyrrolo[3,4-d]pyrimidin-7-one;
2-(4-acetylpiperazin-1-yl)-4-{[(1R,2R)-2-(4-chlorophenyl)cyclopentyl]amino}-6-(1-methylethyl)-5,6-dihydro-7H-pyrrolo[3,4-d]pyrimidin-7-one;
2-(4-acetylpiperazin-1-yl)-4-{[1-(4-fluorophenyl)ethyl]amino}-6-(1-methylethyl)-5,6-dihydro-7H-pyrrolo[3,4-d]pyrimidin-7-one;
2-(4-acetylpiperazin-1-yl)-4-{[1-(5-chloropyridin-2-yl)ethyl]amino}-6-(1-methylethyl)-5,6-dihydro-7H-pyrrolo[3,4-d]pyrimidin-7-one;
2-{[2-(dimethylamino)ethyl]amino}-6-(1-methylethyl)-4-[(2-methyl-1-phenylpropyl)amino]-5,6-dihydro-7H-pyrrolo[3,4-d]pyrimidin-7-one;
2-{[2-(dimethylamino)ethyl]amino}-6-(1-methylethyl)-4-({2-methyl-1-[4-(2-methylpropyl)phenyl]propyl}amino)-5,6-dihydro-7H-pyrrolo[3,4-d]pyrimidin-7-one;
2-(4-acetylpiperazin-1-yl)-4-[3-(4-chlorophenyl)pyrrolidin-1-yl]-6-(1-methylethyl)-5,6-dihydro-7H-pyrrolo[3,4-d]pyrimidin-7-one;
2-(4-acetylpiperazin-1-yl)-4-[(6-fluoro-1,2,3,4-tetrahydronaphthalen-2-yl)amino]-6-(1-methylethyl)-5,6-dihydro-7H-pyrrolo[3,4-d]pyrimidin-7-one;
4-{[bis(4-methoxyphenyl)methyl]amino}-2-{[2-(dimethylamino)ethyl]amino}-6-(1-methylethyl)-5,6-dihydro-7H-pyrrolo[3,4-d]pyrimidin-7-one;
2-(4-acetylpiperazin-1-yl)-4-{[1-(1-ethyl-5-methyl-1H-pyrazol-4-yl)ethyl]amino}-6-(1-methylethyl)-5,6-dihydro-7H-pyrrolo[3,4-d]pyrimidin-7-one;
4-{[2-(4-tert-butylphenyl)ethyl]amino}-2-{[2-(dimethylamino)ethyl]amino}-6-(1-methylethyl)-5,6-dihydro-7H-pyrrolo[3,4-d]pyrimidin-7-one;
2-(4-acetylpiperazin-1-yl)-4-{[2-(4-tert-butylphenyl)ethyl]amino}-6-(1-methylethyl)-5,6-dihydro-7H-pyrrolo[3,4-d]pyrimidin-7-one;
2-(4-acetylpiperazin-1-yl)-4-{[(1S)-1-(4-methoxyphenyl)ethyl]amino}-6-(1-methylethyl)-5,6-dihydro-7H-pyrrolo[3,4-d]pyrimidin-7-one;
2-{[2-(dimethylamino)ethyl]amino}-4-{[2-(2,4-dimethylphenyl)ethyl]amino}-6-(1-methylethyl)-5,6-dihydro-7H-pyrrolo[3,4-d]pyrimidin-7-one;
2-{[2-(dimethylamino)ethyl]amino}-6-(1-methylethyl)-4-{[2-(3-methylphenyl)ethyl]amino}-5,6-dihydro-7H-pyrrolo[3,4-d]pyrimidin-7-one;
2-{[2-(dimethylamino)ethyl]amino}-4-{[2-(3,4-dimethylphenyl)ethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-{[2-(3-methylphenyl)ethyl]amino}-5,6-dihydro-7H-pyrrolo[3,4-d]pyrimidin-7-one;
2-{[2-(dimethylamino)ethyl]amino}-6-(1-methylethyl)-4-{[4-(1-methylethyl)benzyl]amino}-5,6-dihydro-7H-pyrrolo[3,4-d]pyrimidin-7-one;
4-{[2-(4-chlorophenyl)ethyl]amino}-2-{[2-(dimethylamino)ethyl]amino}-6-(1-methylethyl)-5,6-dihydro-7H-pyrrolo[3,4-d]pyrimidin-7-one;
2-(4-acetylpiperazin-1-yl)-4-[benzyl(prop-2-en-1-yl)amino]-6-(1-methylethyl)-5,6-dihydro-7H-pyrrolo[3,4-d]pyrimidin-7-one;
2-{[2-(dimethylamino)ethyl]amino}-6-(1-methylethyl)-4-[methyl(4-methylbenzyl)amino]-5,6-dihydro-7H-pyrrolo[3,4-d]pyrimidin-7-one;
4-[benzyl(ethyl)amino]-2-{[2-(dimethylamino)ethyl]amino}-6-(1-methylethyl)-5,6-dihydro-7H-pyrrolo[3,4-d]pyrimidin-7-one;
2-{[2-(dimethylamino)ethyl]amino}-6-(1-methylethyl)-4-[(2-phenylpropyl)amino]-5,6-dihydro-7H-pyrrolo[3,4-d]pyrimidin-7-one;
4-[benzyl(methyl)amino]-2-{[2-(dimethylamino)ethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-[(2-morpholin-4-yl-2-phenylethyl)amino]-5,6-dihydro-7H-pyrrolo[3,4-d]pyrimidin-7-one;
2-{[2-(dimethylamino)ethyl]amino}-4-[(2,2-diphenylethyl)amino]-6-(1-methylethyl)-5,6-dihydro-7H-pyrrolo[3,4-d]pyrimidin-7-one;
2-[(2-aminoethyl)amino]-4-[(2,2-diphenylethyl)amino]-6-(1-methylethyl)-5,6-dihydro-7H-pyrrolo[3,4-d]pyrimidin-7-one;
2-(4-acetylpiperazin-1-yl)-6-(1-methylethyl)-4-[(1-phenylpropyl)amino]-5,6-dihydro-7H-pyrrolo[3,4-d]pyrimidin-7-one;
2-(4-acetylpiperazin-1-yl)-4-{[2-(dimethylamino)-2-phenylethyl]amino}-6-(1-methylethyl)-5,6-dihydro-7H-pyrrolo[3,4-d]pyrimidin-7-one;
2-{[2-(4-acetylpiperazin-1-yl)-6-(1-methylethyl)-7-oxo-6,7-dihydro-5H-pyrrolo[3,4-d]pyrimidin-4-yl]amino}-2-phenylacetamide;
2-(4-acetylpiperazin-1-yl)-4-{benzyl[2-(dimethylamino)ethyl]amino}-6-(1-methylethyl)-5,6-dihydro-7H-pyrrolo[3,4-d]pyrimidin-7-one;
2-(4-acetylpiperazin-1-yl)-4-{[2-(dimethylamino)-1-phenylethyl] (methyl)amino}-6-(1-methylethyl)-5,6-dihydro-7H-pyrrolo[3,4-d]pyrimidin-7-one;
2-(4-acetylpiperazin-1-yl)-4-{[2-(dimethylamino)-1-phenylethyl]amino}-6-(1-methylethyl)-5,6-dihydro-7H-pyrrolo[3,4-d]pyrimidin-7-one;
2-(4-acetylpiperazin-1-yl)-6-(1-methylethyl)-4-[(2-methyl-2-phenylpropyl)amino]-5,6-dihydro-7H-pyrrolo[3,4-d]pyrimidin-7-one;
4-[(2,2-diphenylethyl)amino]-6-(1-methylethyl)-2-[(2-pyrrolidin-1-ylethyl)amino]-5,6-dihydro-7H-pyrrolo[3,4-d]pyrimidin-7-one;
2-(4-acetylpiperazin-1-yl)-4-(tert-butylamino)-6-(1-methylethyl)-5,6-dihydro-7H-pyrrolo[3,4-d]pyrimidin-7-one;
2-(4-acetylpiperazin-1-yl)-4-[(4-methylbenzyl)amino]-6-(1-methylethyl)-5,6-dihydro-7H-pyrrolo[3,4-d]pyrimidin-7-one;
2-(4-acetylpiperazin-1-yl)-6-(1-methylethyl)-4-(phenylamino)-5,6-dihydro-7H-pyrrolo[3,4-d]pyrimidin-7-one;
2-(4-acetylpiperazin-1-yl)-6-(1-methylethyl)-4-[(2-phenylethyl)amino]-5,6-dihydro-7H-pyrrolo[3,4-d]pyrimidin-7-one;
2-(4-acetylpiperazin-1-yl)-6-(1-methylethyl)-4-[(pyridin-3-ylmethyl)amino]-5,6-dihydro-7H-pyrrolo[3,4-d]pyrimidin-7-one;
2-(4-acetylpiperazin-1-yl)-4-[(3-methoxybenzyl)amino]-6-(1-methylethyl)-5,6-dihydro-7H-pyrrolo[3,4-d]pyrimidin-7-one;
2-(4-acetylpiperazin-1-yl)-4-(benzylamino)-6-(1-methylethyl)-5,6-dihydro-7H-pyrrolo[3,4-d]pyrimidin-7-one;
2-(4-acetylpiperazin-1-yl)-4-[(diphenylmethyl)(methyl)amino]-6-(1-methylethyl)-5,6-dihydro-7H-pyrrolo[3,4-d]pyrimidin-7-one;
2-(4-acetylpiperazin-1-yl)-6-(1-methylethyl)-4-[(1-methyl-1-phenylethyl)amino]-5,6-dihydro-7H-pyrrolo[3,4-d]pyrimidin-7-one;
2-(4-acetylpiperazin-1-yl)-6-(1-methylethyl)-4-[(1-phenylethyl)amino]-5,6-dihydro-7H-pyrrolo[3,4-d]pyrimidin-7-one;
2-amino-4-[(diphenylmethyl)amino]-6-(1-methylethyl)-5,6-dihydro-7H-pyrrolo[3,4-d]pyrimidin-7-one;
6-cyclobutyl-4-[(diphenylmethyl)amino]-2-morpholin-4-yl-5,6-dihydro-7H-pyrrolo[3,4-d]pyrimidin-7-one;
4-[(diphenylmethyl)amino]-2-morpholin-4-yl-6-(tetrahydro-2H-pyran-4-yl)-5,6-dihydro-7H-pyrrolo[3,4-d]pyrimidin-7-one;
4-(2,5-dihydro-1H-pyrrol-1-yl)-6-(1-methylethyl)-2-morpholin-4-yl-5,6-dihydro-7H-pyrrolo[3,4-d]pyrimidin-7-one;
4-[(2-fluoro-5-methylphenyl)amino]-6-(1-methylethyl)-2-morpholin-4-yl-5,6-dihydro-7H-pyrrolo[3,4-d]pyrimidin-7-one;
6-(1-methylethyl)-2-morpholin-4-yl-4-(2-pyridin-2-ylpyrrolidin-1-yl)-5,6-dihydro-7H-pyrrolo[3,4-d]pyrimidin-7-one;
4-[4-(2-methoxyethyl)piperazin-1-yl]-6-(1-methylethyl)-2-morpholin-4-yl-5,6-dihydro-7H-pyrrolo[3,4-d]pyrimidin-7-one;
6-(1-methylethyl)-2-morpholin-4-yl-4-[4-(tetrahydrofuran-2-ylmethyl)piperazin-1-yl]-5,6-dihydro-7H-pyrrolo[3,4-d]pyrimidin-7-one;
6-(1-methylethyl)-2-morpholin-4-yl-4-[(2-pyridin-2-ylethyl)amino]-5,6-dihydro-7H-pyrrolo[3,4-d]pyrimidin-7-one;
4-(4-ethylpiperazin-1-yl)-6-(1-methylethyl)-2-morpholin-4-yl-5,6-dihydro-7H-pyrrolo[3,4-d]pyrimidin-7-one;
4-[3-(diethylamino)pyrrolidin-1-yl]-6-(1-methylethyl)-2-morpholin-4-yl-5,6-dihydro-7H-pyrrolo[3,4-d]pyrimidin-7-one;
4-[4-(2-hydroxyethyl)piperazin-1-yl]-6-(1-methylethyl)-2-morpholin-4-yl-5,6-dihydro-7H-pyrrolo[3,4-d]pyrimidin-7-one;
4-{[4-(dimethylamino)phenyl]amino}-6-(1-methylethyl)-2-morpholin-4-yl-5,6-dihydro-7H-pyrrolo[3,4-d]pyrimidin-7-one;
4-(4-methyl-1,4-diazepan-1-yl)-6-(1-methylethyl)-2-morpholin-4-yl-5,6-dihydro-7H-pyrrolo[3,4-d]pyrimidin-7-one;
4-[2-(methoxymethyl)pyrrolidin-1-yl]-6-(1-methylethyl)-2-morpholin-4-yl-5,6-dihydro-7H-pyrrolo[3,4-d]pyrimidin-7-one;
4-[4-(hydroxymethyl)piperidin-1-yl]-6-(1-methylethyl)-2-morpholin-4-yl-5,6-dihydro-7H-pyrrolo[3,4-d]pyrimidin-7-one;
4-[4-(2,3-dichlorophenyl)piperazin-1-yl]-6-(1-methylethyl)-2-morpholin-4-yl-5,6-dihydro-7H-pyrrolo[3,4-d]pyrimidin-7-one;
4-{[2-(dimethylamino)ethyl] (methyl)amino}-6-(1-methylethyl)-2-morpholin-4-yl-5,6-dihydro-7H-pyrrolo[3,4-d]pyrimidin-7-one;
6-(1-methylethyl)-4-[methyl(pyridin-3-ylmethyl)amino]-2-morpholin-4-yl-5,6-dihydro-7H-pyrrolo[3,4-d]pyrimidin-7-one;
4-[(3R)-3-(dimethylamino)pyrrolidin-1-yl]-6-(1-methylethyl)-2-morpholin-4-yl-5,6-dihydro-7H-pyrrolo[3,4-d]pyrimidin-7-one;
6-(1-methylethyl)-4-[methyl(1-methylpyrrolidin-3-yl)amino]-2-morpholin-4-yl-5,6-dihydro-7H-pyrrolo[3,4-d]pyrimidin-7-one;
4-{[2-(dimethylamino)ethyl](ethyl)amino}-6-(1-methylethyl)-2-morpholin-4-yl-5,6-dihydro-7H-pyrrolo[3,4-d]pyrimidin-7-one;
6-(1-methylethyl)-2-morpholin-4-yl-4-[(pyridin-4-ylmethyl)amino]-5,6-dihydro-7H-pyrrolo[3,4-d]pyrimidin-7-one;
6-(1-methylethyl)-2-morpholin-4-yl-4-[(pyridin-2-ylmethyl)amino]-5,6-dihydro-7H-pyrrolo[3,4-d]pyrimidin-7-one;
4-{[2-(1H-imidazol-4-yl)ethyl]amino}-6-(1-methylethyl)-2-morpholin-4-yl-5,6-dihydro-7H-pyrrolo[3,4-d]pyrimidin-7-one;
4-[(2-hydroxyethyl)amino]-6-(1-methylethyl)-2-morpholin-4-yl-5,6-dihydro-7H-pyrrolo[3,4-d]pyrimidin-7-one;
6-(1-methylethyl)-4-[(1-methyl-1H-indazol-5-yl)amino]-2-morpholin-4-yl-5,6-dihydro-7H-pyrrolo[3,4-d]pyrimidin-7-one;
N-(4-{[6-(1-methylethyl)-2-morpholin-4-yl-7-oxo-6,7-dihydro-5H-pyrrolo[3,4-d]pyrimidin-4-yl]amino}phenyl)acetamide;
N-(4-methoxy-3-{[6-(1-methylethyl)-2-morpholin-4-yl-7-oxo-6,7-dihydro-5H-pyrrolo[3,4-d]pyrimidin-4-yl]amino}phenyl)acetamide;
6-(1-methylethyl)-2-morpholin-4-yl-4-[(4-pyrrolidin-1-ylphenyl)amino]-5,6-dihydro-7H-pyrrolo[3,4-d]pyrimidin-7-one;
6-(1-methylethyl)-2-morpholin-4-yl-4-{[4-(1H-pyrazol-1-yl)phenyl]amino}-5,6-dihydro-7H-pyrrolo[3,4-d]pyrimidin-7-one;
4-{[2-(benzyloxy)phenyl]amino}-6-(1-methylethyl)-2-morpholin-4-yl-5,6-dihydro-7H-pyrrolo[3,4-d]pyrimidin-7-one;
4-[(4-methoxy-2-methylphenyl)amino]-6-(1-methylethyl)-2-morpholin-4-yl-5,6-dihydro-7H-pyrrolo[3,4-d]pyrimidin-7-one;
N-(3-{[6-(1-methylethyl)-2-morpholin-4-yl-7-oxo-6,7-dihydro-5H-pyrrolo[3,4-d]pyrimidin-4-yl]amino}phenyl)propanamide;
4-[(3-methoxyphenyl)amino]-6-(1-methylethyl)-2-morpholin-4-yl-5,6-dihydro-7H-pyrrolo[3,4-d]pyrimidin-7-one;
4-[(6-methoxypyridin-3-yl)amino]-6-(1-methylethyl)-2-morpholin-4-yl-5,6-dihydro-7H-pyrrolo[3,4-d]pyrimidin-7-one;
4-(2,3-dihydro-1,4-benzodioxin-6-ylamino)-6-(1-methylethyl)-2-morpholin-4-yl-5,6-dihydro-7H-pyrrolo[3,4-d]pyrimidin-7-one;
4-(1H-indol-5-ylamino)-6-(1-methylethyl)-2-morpholin-4-yl-5,6-dihydro-7H-pyrrolo[3,4-d]pyrimidin-7-one;
4-{[3-(dimethylamino)propyl]amino}-6-(1-methylethyl)-2-morpholin-4-yl-5,6-dihydro-7H-pyrrolo[3,4-d]pyrimidin-7-one;
6-(1-methylethyl)-2-morpholin-4-yl-4-[(6-morpholin-4-ylpyridin-3-yl)amino]-5,6-dihydro-7H-pyrrolo[3,4-d]pyrimidin-7-one;
6-(1-methylethyl)-2-morpholin-4-yl-4-[(6-phenoxypyridin-3-yl)amino]-5,6-dihydro-7H-pyrrolo[3,4-d]pyrimidin-7-one;
6-(1-methylethyl)-2-morpholin-4-yl-4-{[3-(1H-pyrrol-1-yl)phenyl]amino}-5,6-dihydro-7H-pyrrolo[3,4-d]pyrimidin-7-one;
4-[(3-benzylphenyl)amino]-6-(1-methylethyl)-2-morpholin-4-yl-5,6-dihydro-7H-pyrrolo[3,4-d]pyrimidin-7-one;
4-[(3-fluoro-4-methoxyphenyl)amino]-6-(1-methylethyl)-2-morpholin-4-yl-5,6-dihydro-7H-pyrrolo[3,4-d]pyrimidin-7-one;
4-[(1-acetyl-2,3-dihydro-1H-indol-6-yl)amino]-6-(1-methylethyl)-2-morpholin-4-yl-5,6-dihydro-7H-pyrrolo[3,4-d]pyrimidin-7-one;
6-(1-methylethyl)-2-morpholin-4-yl-4-[(2-morpholin-4-ylphenyl)amino]-5,6-dihydro-7H-pyrrolo[3,4-d]pyrimidin-7-one;
6-(1-methylethyl)-4-[(2-methylphenyl)amino]-2-morpholin-4-yl-5,6-dihydro-7H-pyrrolo[3,4-d]pyrimidin-7-one;
N-(3-{[6-(1-methylethyl)-2-morpholin-4-yl-7-oxo-6,7-dihydro-5H-pyrrolo[3,4-d]pyrimidin-4-yl]amino}phenyl)acetamide;
6-(1-methylethyl)-2-morpholin-4-yl-4-[(4-morpholin-4-ylphenyl)amino]-5,6-dihydro-7H-pyrrolo[3,4-d]pyrimidin-7-one;
6-(1-methylethyl)-2-morpholin-4-yl-4-[(2-pyrrolidin-1-ylethyl)amino]-5,6-dihydro-7H-pyrrolo[3,4-d]pyrimidin-7-one;
N-(2-{[6-(1-methylethyl)-2-morpholin-4-yl-7-oxo-6,7-dihydro-5H-pyrrolo[3,4-d]pyrimidin-4-yl]amino}phenyl)acetamide;
4-(1,3-benzodioxol-5-ylamino)-6-(1-methylethyl)-2-morpholin-4-yl-5,6-dihydro-7H-pyrrolo[3,4-d]pyrimidin-7-one;
4-[(2-methoxyphenyl)amino]-6-(1-methylethyl)-2-morpholin-4-yl-5,6-dihydro-7H-pyrrolo[3,4-d]pyrimidin-7-one;
5-(1-{4-[(4-chlorophenyl)amino]-6-(1-methylethyl)-7-oxo-6,7-dihydro-5H-pyrrolo[3,4-d]pyrimidin-2-yl}piperidin-4-yl)imidazolidine-2,4-dione;
4-[(4-chlorophenyl)amino]-6-(1,3-dimethylbutyl)-2-morpholin-4-yl-5,6-dihydro-7H-pyrrolo[3,4-d]pyrimidin-7-one;
4-[(4-chlorophenyl)amino]-2-(3-hydroxypyrrolidin-1-yl)-6-(1-methylethyl)-5,6-dihydro-7H-pyrrolo[3,4-d]pyrimidin-7-one;
4-[(3,4-difluorophenyl)amino]-6-(1-methylethyl)-2-morpholin-4-yl-5,6-dihydro-7H-pyrrolo[3,4-d]pyrimidin-7-one;
4-{4-[(4-chlorophenyl)amino]-6-(1-methylethyl)-7-oxo-6,7-dihydro-5H-pyrrolo[3,4-d]pyrimidin-2-yl}piperazine-1-carbaldehyde;
4-[(3-fluoro-4-methylphenyl)amino]-6-(1-methylethyl)-2-morpholin-4-yl-5,6-dihydro-7H-pyrrolo[3,4-d]pyrimidin-7-one;
1-{4-[(4-chlorophenyl)amino]-6-(1-methylethyl)-7-oxo-6,7-dihydro-5H-pyrrolo[3,4-d]pyrimidin-2-yl}piperidine-4-carboxylic acid;
6-(1-methylethyl)-2-morpholin-4-yl-4-{[4-(1,3-oxazol-2-yl)phenyl]amino}-5,6-dihydro-7H-pyrrolo[3,4-d]pyrimidin-7-one;
4-[(3-chloro-4-methylphenyl)amino]-6-(1-methylethyl)-2-morpholin-4-yl-5,6-dihydro-7H-pyrrolo[3,4-d]pyrimidin-7-one;
4-[(4-chlorophenyl)amino]-6-(1-methylethyl)-2-thiomorpholin-4-yl-5,6-dihydro-7H-pyrrolo[3,4-d]pyrimidin-7-one;
6-(1-methylethyl)-2-morpholin-4-yl-4-{[4-(phenylsulfonyl)phenyl]amino}-5,6-dihydro-7H-pyrrolo[3,4-d]pyrimidin-7-one;
4-[(4-chlorophenyl)(methyl)amino]-6-(1-methylethyl)-2-morpholin-4-yl-5,6-dihydro-7H-pyrrolo[3,4-d]pyrimidin-7-one;
6-(1-methylheptyl)-2-morpholin-4-yl-4-piperidin-1-yl-5,6-dihydro-7H-pyrrolo[3,4-d]pyrimidin-7-one;
4-(diethylamino)-6-(1-ethynylcyclohexyl)-2-pyrrolidin-1-yl-5,6-dihydro-7H-pyrrolo[3,4-d]pyrimidin-7-one;
6-cyclohexyl-4-(diethylamino)-2-pyrrolidin-1-yl-5,6-dihydro-7H-pyrrolo[3,4-d]pyrimidin-7-one;
4-[(4-chlorophenyl)amino]-6-(1-methylethyl)-2-morpholin-4-yl-5,6-dihydro-7H-pyrrolo[3,4-d]pyrimidin-7-one;
4-(cyclohexylamino)-6-(1-methylethyl)-2-morpholin-4-yl-5,6-dihydro-7H-pyrrolo[3,4-d]pyrimidin-7-one;
6-(1-methylethyl)-2-morpholin-4-yl-4-{[4-(trifluoromethyl)phenyl]amino}-5,6-dihydro-7H-pyrrolo[3,4-d]pyrimidin-7-one;
4-[(4-fluorophenyl)amino]-6-(1-methylethyl)-2-morpholin-4-yl-5,6-dihydro-7H-pyrrolo[3,4-d]pyrimidin-7-one;
4-(biphenyl-4-ylamino)-6-(1-methylethyl)-2-morpholin-4-yl-5,6-dihydro-7H-pyrrolo[3,4-d]pyrimidin-7-one;
4-[(4-chlorophenyl)amino]-6-cyclobutyl-2-morpholin-4-yl-5,6-dihydro-7H-pyrrolo[3,4-d]pyrimidin-7-one;
4-[(4-chlorophenyl)amino]-6-cyclopentyl-2-morpholin-4-yl-5,6-dihydro-7H-pyrrolo[3,4-d]pyrimidin-7-one;
4-[(4-chlorophenyl)amino]-6-cyclopropyl-2-morpholin-4-yl-5,6-dihydro-7H-pyrrolo[3,4-d]pyrimidin-7-one;
ethyl 1-{4-[(4-chlorophenyl)amino]-6-(1-methylethyl)-7-oxo-6,7-dihydro-5H-pyrrolo[3,4-d]pyrimidin-2-yl}piperidine-4-carboxylate;
6-(1-methylethyl)-2-morpholin-4-yl-4-(4-phenylpiperidin-1-yl)-5,6-dihydro-7H-pyrrolo[3,4-d]pyrimidin-7-one;
4-[(4-bromophenyl)amino]-6-(1-methylethyl)-2-morpholin-4-yl-5,6-dihydro-7H-pyrrolo[3,4-d]pyrimidin-7-one; and
6-(1-methylethyl)-2-morpholin-4-yl-4-{[4-(phenylsulfanyl)phenyl]amino}-5,6-dihydro-7H-pyrrolo[3,4-d]pyrimidin-7-one.

### EXAMPLES OF THE INVENTION

The invention will further be described in more detail by the following Examples which describe methods whereby compounds of the present invention may be prepared, purified, analyzed and biologically tested, and which are not to be construed as limiting the invention.
Ac acetyl
AcOH acetic acid
AIBN azobisisobutylonitrile
am u atomic mass unit
aq aqueous
atm atmosphere
Bu butyl
CDI carbonyl diimidazole
Conc concentrated
d doublet
dd doublet of doublet
ddd doublet of doublet of doublet
DCE 1,2-dichloroethane
DIBAL diisobutylaluminum hydride
DME dimethyoxyethane
DMF N,N-dimethyl formamide
DMSO dimethylsulfoxide
DMSO-d₆ dimethylsulfoxide-d₆
EDCI 1-(3-dimethylaminopropyl)-3-ethylcarbodiimide hydrochloride
El electron impact ionization
EI-MS electron impact-mass spectrometry equiv equivalent
ES-MS electrospray mass spectrometry
Et ethyl
Et₂O diethyl ether
Et₃N triethylamine
EtOAc ethyl acetate
EtOH ethanol
Ex example
g gram
h hour(s)
Hex hexanes
¹H NMR proton nuclear magnetic resonance
HATU O-(7-azabenzotriazol-1-yl)-N,N,N′,N′-tetramethyluronium hexafluorophosphate
HBTU 2-(1H-Benzotriazole-1-yl-1,1,3,3-tetramethyluronium hexafluorophosphate
HOAT 1-hydroxy-7-aza-benzotriazole
HOBT 1-hydroxybenzotriazole
HPLC high-performance liquid chromatography
HPLC ES-MS high-performance liquid chromatography-electrospray mass spectroscopy
L liter
LCMS liquid chromatography/mass spectroscopy
LHMDS lithium bis(trimethylsilyl)amide m multiplet
M molar
mL milliliter
m/v mass over charge
Me methyl
MeCN acetonitrile
MeOH methanol
mg milligram
MHz megahertz
min minute(s)
mmol millimole mol mole
mp melting point
MS mass spectrometry
N normal
NBS N-bromosuccinimide
Pd₂(dba)₃ tris(dibenzylideneacetone)dipalladium(0)
Pd(OAc)₂ palladium acetate
Pd(PPh₃)₄ tetrakis(triphenylphosphine)palladium(0)
Pd/C palladium on carbon
Pd(dppf)Cl_{2 [}1,1′-bis(diphenylphosphino)ferrocene]dichloropalladium(II)
Ph phenyl
ppm parts per million
Pr propyl
psi pounds per square inch
q quartet
qt quintet
Rf TLC retention factor
rt room temperature
RT retention time (HPLC)
SFC supercritical-fluid chromatography
s singlet
t triplet
TBAF tetrabutylammonium fluoride
TBDMS tert-butyldimethylsilyl
TBDMSCI tert-butyldimethylsilyl chloride
TBS tert-butyldimethylsilyl
TFA trifluoroacetic acid
THF tetrahydrofuran
TLC thin layer chromatography
TMS tetramethylsilane
v/v volume per unit volume
vol volume
w/w weight per unit weight

### General Experimental Methods

All starting materials are commercially available or described in the literature. Air and moisture sensitive liquids and solutions were transferred via syringe or cannula, and introduced into reaction vessels through rubber septa. Commercial grade reagents and solvents were used without further purification. The terms “concentration under reduced pressure” and “in vacuo” refer to use of a Buchi rotary evaporator at approximately 15 mm of Hg. All temperatures are reported uncorrected in degrees Celsius (0° C.). Thin layer chromatography (TLC) was performed on EM Science pre-coated glass-backed silica gel 60 Å F-254 250 μm plates. Column chromatography (flash chromatography) was performed using 32-63 micron, 60 Å, silica gel prepacked cartridges (on a Biotage or ISCO system) or using glass column and air pressure. The ¹H NMR spectra were recorded on Varian at 400 MHz. The mass spectra were recorded utilising electrospray (LC-MS; column XTerra MS C8 2.5 μm 2.1×30 mm, buffer gradient H₂O 0.1% TFA: CH₃CN+0.04% TFA, MS: micromass ZMD/ammonium acetate buffer) ionisation techniques. The preparative HPLC or LCMS (high pH or low pH) was performed using a Waters X-bridge Prep C₁₈ OBD, 30×50 mm, 5 μm particle size, Mobile phase: A=Water 10 mM NH₄HCO₃ (pH 10) or Water 0.1% TFA and B: MeCN. SFC (supercritical-fluid chromatography) was performed using a MinGram SFC instrument from Mettler Toledo. Flow Rate: 10 mL/min. Columns: 10×250 mm, 5 μm particle size, ChiralCel OD-H or OJ-H columns or ChiralPak AS-H column. Eluents: Main eluent is CO₂, with MeOH or i-PrOH or EtOH+0.1% Dimethylethylamine (DMEA) or Isorpopanol+0.1% DMEA as a modifier. Column Temperature: 35° C. Back Pressure Regulator set to 100 Bar. Detection: UV detection at wavelength 215 nm.

### Intermediate 1

### 2,4-dihydroxy-5H-furo[3,4-d]pyrimidine-7-one

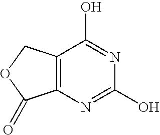

Orotic acid (20.4 g, 130.7 mmol) and paraformaldehyde (15.5 g, 523.0 mmol) were taken in a 1000 mL round-bottom flask to which concentrated HCl (420 mL, 9680 mmol) was added. The reaction mixture was refluxed at 85-95° C. for 18 h using a condenser with ethylene glycol. After cooling, HCl was evaporated under reduced pressure to obtain a solid. Water (200 mL) was then added and volatiles were evaporated under reduced pressure. The white solid obtained was digested with water (150 mL) at 65-70° C. for 1 h after which the solid was filtered to afford the title compound as a solid (17 g, 77%). ¹H NMR (DMSO-d₆) δ ppm 5.10 (s, 2H), 11.50 (s, 1H), 12.10 (s, 1H).

### Intermediate 2

### 2,4-dihydroxy-6-isopropyl-5,6-dihydropyrrolo[3,4-d]pyrimidine-7-one

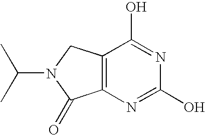

To a stirred suspension of 2,4-dihydroxy-5H-furo[3,4-d]pyrimidine-7-one (Intermediate 1, 5.06 g, 30.1 mmol) in 2-methoxyethanol (40 mL) kept in a 350 mL glass pressure vessel, was added isopropyl amine hydrochloride (5.76 g, 60.2 mmol). The reaction mixture was heated at 190-200° C. for 6 h. Alternatively, the reaction mixture could be heated in a microwave at 200° C. for 2 h. The reaction mixture was allowed to slowly cool to rt overnight. The solvent was evaporated under reduced pressure then 35 mL of crushed ice/water was added. The orange/yellow solid obtained was filtered, washed with 15 mL of cold H₂O and dried to give the title compound (3.4 g, 54%). ¹H NMR (300 MHz, DMSO-d₆) δ ppm 1.10 (d, 6H), 4.10 (s, 2H), 4.30-4.50 (m, 1H), 11.20 (s, 1H), 11.80 (s, 1H).

Alternatively, 2,4-Dihydroxy-6-isopropyl-5,6-dihydropyrrolo[3,4-d]pyrimidine-7-one (Intermediate 2) can be prepared in large scale following a procedure as shown below:

Denatured ethanol (4 L) and 37% formaldehyde (551 mL, 7.4 mol) were added to orotic acid monohydrate (258 g, 1.48 mol). Isopropylamine (630 mL, 7.4 mol) was then slowly added to the suspension with stirring. The addition was slightly exothermic. The mixture was refluxed for 16 h. The reaction mixture was cooled in an ice bath, the white solid was collected by filtration and washed with ethanol to give the crude intermediate (409 g) as a solid. ¹H NMR (300 MHz, DMSO-d₆) δ ppm 1.20 (d, J=6.5 Hz, 6H), 3.24 (qu, J=6.6 Hz, 1H), 3.90 (s, 2H). To the above crude intermediate (409 g) was added 2-methoxyethanol (1.9 L) and 12 N hydrochloric acid (190 mL). The reaction mixture was refluxed for 16 h, cooled in an ice bath and the solid was collected by filtration and washed with ethanol to give the title compound (228 g, 73% over two steps) as a solid. ¹H NMR (300 MHz, DMSO-d₆) δ ppm 1.17 (d, J=6.8 Hz, 6H), 4.12 (s, 2H), 4.24 (qu, J=6.7 Hz, 1H), 11.24 (s, 1H), 11.79 (s, 1H).

### Intermediate 3

### 2,4-dichloro-6-isopropyl-5,6-dihydropyrrolo[3,4-d]pyrimidine-7-one

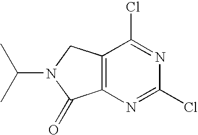

To a suspension of 2,4-Dihydroxy-6-isopropyl-5,6-dihydropyrrolo[3,4-d]pyrimidine-7-one (Intermediate 2) (35.0 g, 0.17 mol) in phosphorus oxychloride (360 mL) was added N,N-diethylaniline (70 mL, 0.44 mol) slowly. The mixture was heated to 80° C. (internal temperature) and kept at that temperature for 2.5 h, and then concentrated under reduced pressure, with toluene co-evaporation to remove any trace of phosphorus oxychloride. The residue was poured onto crushed ice, and the pH of the mixture was adjusted to pH 6 using 30% NH₄OH at 0° C. The mixture was extracted with EtOAc. The organic layer was washed with brine, dried over Na₂SO₄, filtered and concentrated under reduced pressure. The residue was purified by silica gel chromatography (hexanes:EtOAc 4:1 to 2:3) to afford the title compound (24.2 g, 59%) as a solid. Mp=122-123° C. ¹H NMR (300 MHz, CDCl₃) δ ppm 1.34 (d, J=6.8 Hz, 6H), 4.41 (s, 2H), 4.74 (td, J=6.8, 13.6 Hz, 1H). ¹³C NMR (75 MHz, CDCl₃) δ ppm 20.8, 41.9, 44.4, 130.5, 158.3, 162.1, 162.2, 163.1.

### Intermediate 4

### tetrahydro-2H-pyran-4-amine

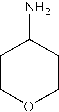

To a solution of tetrahydro-4H-pyran-4-one (100 g, 1 mol) in MeCN (3 L) were added benzylamine (109 mL, 1 mol), NaBH(OAc)₃ (296 g, 1.40 mol) and AcOH (60 mL). The resulting solution was stirred for 16 h at rt. MeCN was removed under reduced pressure and EtOAc (1 L) was added. The solution was washed with 1 N NaOH (500 mL), brine (500 mL) and dried over anhydrous MgSO₄, filtered and concentrated under reduced pressure to afford N-benzyltetrahydro-2H-pyran-4-amine (191.4 g, quant.). ¹H NMR (300 MHz, CDCl₃) δ ppm 1.38-1.52 (m, 3H), 1.86 (d, J=12.7 Hz, 2H), 2.68-2.78 (m, 1H), 3.39 (t, J=10.8 Hz, 2H), 3.83 (s, 2H), 3.98 (d, J=11.4 Hz, 2H), 7.22-7.36 (m, 5H). To a solution of the above N-benzyltetrahydro-2H-pyran-4-amine (191 g, 1 mol) in EtOH (700 mL) was added 5% Pd/C (38.2 g) in a hydrogenation apparatus bottle. The bottle was filled with 50 psi of H₂ and was shaken for 16 h. The solution was filtered on diatomaceous earth and 5% Pd/C (38.2 g) was added again and the bottle was filled with 50 psi of H₂ and was shaken for 16 h. The reaction mixture was filtered on diatomaceous earth and concentrated under reduced pressure to give a solution of the title amine (101 g, quant.), which was used in the next step. An aliquot was concentrated under reduced pressure for characterization. ¹H NMR (300 MHz, CDCl₃) δ ppm 1.39 (ddd, J=15.8, 12.4, 4.6 Hz, 2H), 1.77 (d, J=11.4 Hz, 2H), 2.81-2.91 (m, 1H), 3.38 (t, J=11.5 Hz, 2H), 3.95 (d, J=11.6 Hz, 2H).

### Intermediate 5

### 2,6-dioxo-5-((tetrahydro-2H-pyran-4-ylamino)methyl)-1,2,3,6-tetrahydropyrimidine-4-carboxylic acid

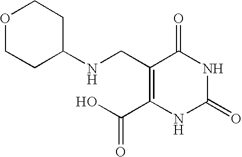

To a solution of orotic acid monohydrate (20.0 g, 0.11 mol) and formaldehyde (51.3 mL, 0.69 mol, 37% in water) in EtOH (1 L) was slowly added tetrahydro-2H-pyran-4-amine (Intermediate 4) (0.69 mol). The resulting solution was refluxed for 16 h. The reaction mixture was cooled to 0° C. and filtered to afford the title compound as a solid (14.4 g, 46%), which was used in the next step without further purification. ¹H NMR (300 MHz, DMSO-d₆) δ ppm 1.44-1.58 (m, 2H), 1.89 (d, J=10.7 Hz, 2H), 3.16-3.32 (m, 4H), 3.87 (dd, J=11.6, 2.5 Hz, 1H), 3.94 (s, 2H), 9.61 (br s, 2H).

### Intermediate 6

### 5-((sec-butylamino)methyl)-2,6-dioxo-1,2,3,6-tetrahydropyrimidine-4-carboxylic acid

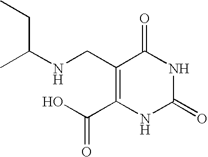

Following a procedure similar to that described for Intermediate 5, the title compound was obtained as a solid (10.2 g, 74%). ¹H NMR (300 MHz, DMSO-d₆) δ ppm 0.89 (t, J=7.5 Hz, 3H), 1.17 (d, J=6.5 Hz, 3H), 1.42-1.66 (m, 2H), 3.05 (q, J=6.5 Hz, 1H), 3.89 (dd, J=17.0, 13.0 Hz, 2H), 10.38 (br s, 1H), 11.33 (br s, 1H).

### Intermediate 7

### (S)-5-((sec-butylamino)methyl)-2,6-dioxo-1,2,3,6-tetrahydropyrimidine-4-carboxylic acid

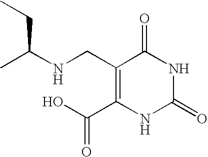

Following a procedure similar to that described for Intermediate 5, the title compound was obtained as a solid (0.5 g, 70%). ¹H NMR (300 MHz, DMSO-d₆) δ ppm 0.90 (t, J=7.5 Hz, 3H), 1.19 (d, J=6.5 Hz, 3H), 1.41-1.56 (m, 1H), 1.58-1.72 (m, 1H), 3.06 (qd, J=13.2, 6.7 Hz, 1H), 3.86-3.96 (m, 2H).

### Intermediate 8

### 5-((3-methylbutan-2-ylamino)methyl)-2,6-dioxo-1,2,3,6-tetrahydropyrimidine-4-carboxylic acid

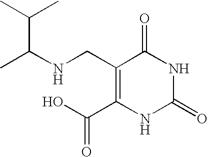

Following a procedure similar to that described for Intermediate 5, the title compound was obtained as a solid (8.0 g, 60%). ¹H NMR (300 MHz, DMSO-d₆) δ ppm 0.89 (t, J=7.0 Hz, 6H), 1.13 (d, J=6.7 Hz, 3H), 1.82-1.93 (m, 1H), 2.97 (br s, 1H), 3.89 (dd, J=22.5, 12.5 Hz, 2H), 9.09 (br s, 1H), 9.81 (br s, 1H), 10.13 (br s, 1H), 11.33 (br s, 1H).

### Intermediate 9

### 5-((1-methoxypropan-2-ylamino)methyl)-2,6-dioxo-1,2,3,6-tetrahydropyrimidine-4-carboxylic acid

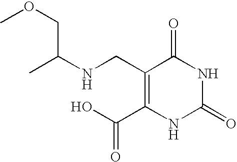

Following a procedure similar to that described for Intermediate 5, the title compound was obtained as a solid (8.55 g, 59%). ¹H NMR (300 MHz, DMSO-d₆) δ ppm 1.17 (d, J=6.5 Hz, 3H), 3.26 (s, 3H), 3.29-3.48 (m, 3H), 3.92 (s, 2H), 9.41 (br s, 1H).

### Intermediate 10

### 5-((2-chlorobenzylamino)methyl)-2,6-dioxo-1,2,3,6-tetrahydropyrimidine-4-carboxylic acid

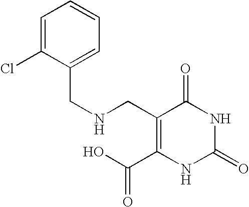

To a mixture of ethanol (50 mL) and 37% formaldehyde (5 mL, 67 mmol), o-chlorobenzylamine (4.2 mL, 35 mmol) was slowly added [Exotherm!]. The mixture was stirred at rt for 16 h. Orotic acid monohydrate (4.5 g, 25.8 mmol) and formaldehyde (5 mL, 42 mmol) were then added and the mixture was heated at reflux for 18 h. After cooling to rt, the white solid was collected by filtration and washed with ethanol. Ethanol was added to the mother liquors and another crop of solid precipitated and was collected and combined to give the title compound (3.5 g, 43%). ¹H-NMR (300 MHz, DMSO-d₆) δ ppm 3.98 (s, 2H), 4.24 (s, 2H), 7.38-7.47 (m, 2H), 7.52-7.55 (m, 1H), 7.59-7.62 (m, 1H), 10.16 (s, 3H), 11.34 (s, 1H).

### Intermediate 11

### 6-(tetrahydro-2H-pyran-4-yl)-5,6-dihydro-1H-pyrrolo[3,4-d]pyrimidine-2,4,7-(3H)-trione

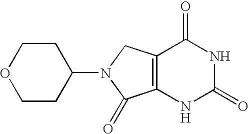

A solution of 2,6-dioxo-5-((tetrahydro-2H-pyran-4-ylamino)methyl)-1,2,3,6-tetrahydropyrimidine-4-carboxylic acid (Intermediate 5) (53.1 mmol) and 12 N HCl (25 mL) in 2-methoxyethanol (150 mL) was refluxed for 16 h. The mixture was cooled to 0° C. and filtered to afford the title compound as a solid (11.4 g, 86%), which was used in the next step without further purification. ¹H NMR (300 MHz, DMSO-d₆) δ ppm 1.62 (dd, J=12.3, 2.6 Hz, 2H), 1.76 (ddd, J=12.3, 12.0, 4.5 Hz, 2H), 3.39 (dt, J=11.5, 1.6 Hz, 2H), 3.89 (dd, J=11.2, 3.9 Hz, 2H), 4.04-4.15 (m, 1H), 4.17 (s, 2H), 11.25 (s, 1H), 11.81 (s, 1H).

### Intermediate 12

### 6-sec-Butyl-5,6-dihydro-1H-pyrrolo[3,4-d]pyrimidine-2,4,7-(3H)-trione

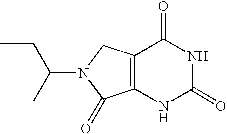

Following a procedure similar to that described for Intermediate 11, starting from 5-((sec-butylamino)methyl)-2,6-dioxo-1,2,3,6-tetrahydropyrimidine-4-carboxylic acid (Intermediate 6), the title compound was obtained as a solid (8.6 g, 91%). ¹H NMR (300 MHz, DMSO-d₆) δ ppm 0.76 (t, J=7.5 Hz, 3H), 1.15 (d, J=7.0 Hz, 3H), 1.51-1.57 (m, 2H), 3.99-4.04 (m, 1H), 4.06 (d, J=7.5 Hz, 2H), 11.25 (s, 1H), 11.80 (s, 1H).

### Intermediate 13

### (S)-6-sec-Butyl-5,6-dihydro-1H-pyrrolo[3,4-d]pyrimidine-2,4,7-(3H)-trione

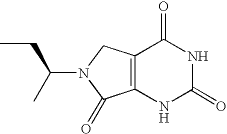

Following a procedure similar to that described for Intermediate 11, starting from (S)-5-((sec-butylamino)methyl)-2,6-dioxo-1,2,3,6-tetrahydropyrimidine-4-carboxylic acid (Intermediate 7), the title compound was obtained as a solid (0.37 g, 80%). ¹H NMR (300 MHz, DMSO-d₆) δ ppm 0.78 (t, J=7.4 Hz, 3H), 1.17 (d, J=6.8 Hz, 3H), 1.42-1.63 (m, 2H), 3.98-4.16 (m, 3H), 11.27 (s, 1H), 11.82 (s, 1H).

### Intermediate 14

### 6-(3-methylbutan-2-yl)-5,6-dihydro-1H-pyrrolo[3,4-d]pyrimidine-2,4,7(3H)-trione

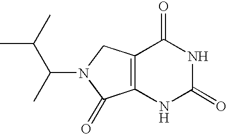

Following a procedure similar to that described for Intermediate 11, starting from 5-((3-methylbutan-2-ylamino)methyl)-2,6-dioxo-1,2,3,6-tetrahydropyrimidine-4-carboxylic acid (Intermediate 8), the title compound was obtained as a solid (5.6 g, 81%). ¹H NMR (300 MHz, DMSO-d₆) δ ppm 0.75 (d, J=6.6 Hz, 3H), 0.94 (d, J=6.7 Hz, 3H), 1.20 (d, J=6.8 Hz, 3H), 1.80-1.87 (m, 1H), 3.76-3.82 (m, 1H), 4.10 (dd, J=32.4, 19.0 Hz, 2H), 11.27 (s, 1H), 11.82 (s, 1H).

### Intermediate 15

### 6-(1-methoxypropan-2-yl)-5,6-dihydro-1H-pyrrolo[3,4-d]pyrimidine-2,4,7(3H)-trione

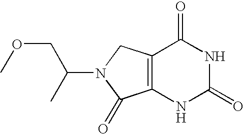

Following a procedure similar to that described for Intermediate 11, starting from 5-((1-methoxypropan-2-ylamino)methyl)-2,6-dioxo-1,2,3,6-tetrahydropyrimidine-4-carboxylic acid (Intermediate 9), the title compound was obtained as a solid (5.5 g, 69%). ¹H NMR (300 MHz, DMSO-d₆) δ ppm 1.13 (d, J=7.0 Hz, 3H), 3.22 (s, 3H), 3.34-3.50 (m, 2H), 4.10 (dd, J=31.2, 19.0 Hz, 2H), 4.28-4.35 (m, 1H), 11.26 (s, 1H), 11.81 (s, 1H).

### Intermediate 16

### 6-(2-Chlorobenzyl)-5,6-dihydro-1H-pyrrolo[3,4-d]pyrimidine-2,4,7(3H)-trione

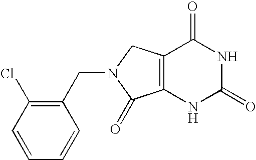

Following a procedure similar to that described for Intermediate 11, starting from 5-((2-chlorobenzylamino)methyl)-2,6-dioxo-1,2,3,6-tetrahydropyrimidine-4-carboxylic acid (Intermediate 10), the title compound was obtained as a solid (1.35 g, 67%). ¹H NMR (300 MHz, DMSO-d₆) δ ppm 4.10 (s, 2H), 4.72 (s, 2H), 7.24-7.37 (m, 3H), 7.45-7.50 (m, 1H), 11.31 (s, 1H), 11.92 (s, 1H). ¹³C NMR (75 MHz, DMSO-d₆) δ ppm 44.6, 46.8, 115.3, 128.3, 130.1, 130.2, 132.9, 134.4, 144.2, 152.6, 161.0, 162.7.

### Intermediate 17

### 2,4-dichloro-6-tetrahydropyran-4-yl-5H-pyrrolo[3,4-d]pyrimidin-7-one

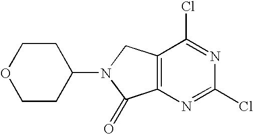

A solution of 6-(tetrahydro-2H-pyran-4-yl)-5,6-dihydro-1H-pyrrolo[3,4-d]pyrimidine-2,4,7(3H)-trione (Intermediate 11) (23.1 mmol) and diethylaniline (4.8 mL, 30.0 mmol) in POCl₃ (60 mL) was heated at 80° C. for 7 h. Then the mixture was cooled to rt and POCl₃ was removed under reduced pressure using toluene (2×20 mL) to ensure complete removal. Ethyl acetate (70 mL) was added, the solution was filtered to recover the starting material. The filtrate was poured in water, washed with brine, dried with anhydrous MgSO₄, filtered and concentrated under reduced pressure. The residue was purified by silica gel chromatography (1:1, hexanes/EtOAc) to give the title compound as a solid (2.0 g, 30%). ¹H NMR (300 MHz, CDCl₃) δ ppm 1.77-1.95 (m, 4H), 3.55 (dt, J=11.6, 3.3 Hz, 2H), 4.10 (dd, J=13.5, 3.4 Hz, 2H), 4.45 (s, 2H), 4.60 (ddd, J=16.2, 10.9, 5.4 Hz, 1H). ¹³C NMR (75 MHz, CDCl₃) δ ppm 31.0, 42.8, 49.4, 67.1, 130.6, 158.4, 162.1, 162.5, 162.7.

### Intermediate 18

### 6-sec-butyl-2,4-dichloro-5H-pyrrolo[3,4-d]pyrimidin-7(6H)-one

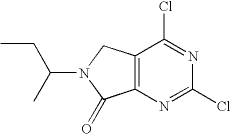

Following a procedure similar to that described for Intermediate 17, starting from 6-sec-Butyl-5,6-dihydro-1H-pyrrolo[3,4-d]pyrimidine-2,4,7(3H)-trione (Intermediate 12) and after purification by silica gel chromatography (6:4, hexanes/EtOAc), the title compound was obtained as a solid (1.3 g, 34%). ¹H NMR (300 MHz, CDCl₃) δ ppm 0.92 (t, J=7.0 Hz, 3H), 1.32 (d, J=7.0 Hz, 3H), 1.68 (sp, J=7.0 Hz, 2H), 4.36 (dd, J=26.0, 18.0 Hz, 2H), 4.51 (q, J=7.5 Hz, 1H). ¹³C NMR (75 MHz, CDCl₃) δ ppm 11.2, 18.9, 28.0, 42.0, 50.1, 130.5, 158.3, 162.1, 162.7, 163.1.

### Intermediate 19

### (S)-6-sec-butyl-2,4-dichloro-5H-pyrrolo[3,4-d]pyrimidin-7(6H)-one

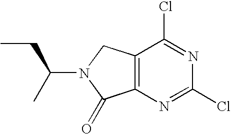

Following a procedure similar to that described for Intermediate 17, starting from (S)-6-sec-Butyl-5,6-dihydro-1H-pyrrolo[3,4-d]pyrimidine-2,4,7(3H)-trione (Intermediate 13) and after purification by silica gel chromatography (7:3 to 6:4, hexanes/EtOAc), the title compound was obtained as an oil (238 mg, 57%). ¹H NMR (300 MHz, CDCl₃) δ ppm 0.92 (t, J=7.4 Hz, 3H), 1.32 (d, J=6.8 Hz, 3H), 1.63-1.74 (m, 2H), 4.29-4.44 (m, 2H), 4.46-4.76 (m, 2H).

### Intermediate 20

### 2,4-dichloro-6-(3-methylbutan-2-yl)-5H-pyrrolo[3,4-d]pyrimidin-7(6H)-one

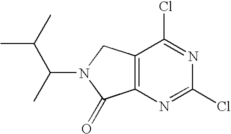

Following a procedure similar to that described for Intermediate 17, starting from 6-(3-methylbutan-2-yl)-5,6-dihydro-1H-pyrrolo[3,4-d]pyrimidine-2,4,7(3H)-trione (Intermediate 14) and after purification by silica gel chromatography (6:4, hexanes/EtOAc), the title compound was obtained as a solid (1.33 g, 21%). ¹H NMR (300 MHz, CDCl₃) δ ppm 0.78 (d, J=6.7 Hz, 3H), 0.97 (d, J=6.6 Hz, 3H), 1.27 (d, J=6.9 Hz, 3H), 1.77-1.86 (m, 1H), 4.08-4.18 (m, 1H), 4.37 (dd, J=13.2, 8.6 Hz, 2H). ¹³C NMR (75 MHz, CDCl₃) δ ppm 17.3, 19.8, 20.1, 32.6, 42.5, 54.6, 130.6, 158.3, 161.9, 162.7, 162.9.

### Intermediate 21

### 2,4-dichloro-6-(1-methoxypropan-2-yl)-5H-pyrrolo[3,4-d]pyrimidin-7(6H)-one

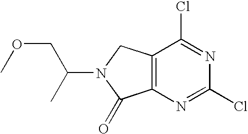

Following a procedure similar to that described for Intermediate 17, starting from 6-(1-methoxypropan-2-yl)-5,6-dihydro-1H-pyrrolo[3,4-d]pyrimidine-2,4,7(3H)-trione (Intermediate 15) and after purification by silica gel chromatography (4:6, hexanes/EtOAc), the title compound was obtained as a solid (3.7 g, 58%). ¹H NMR (300 MHz, CDCl₃) δ ppm 1.37 (d, J=7.1 Hz, 3H), 3.34 (s, 3H), 3.57 (d, J=5.0 Hz, 2H), 4.53 (dd, J=39.1, 18.8 Hz, 2H), 4.71-4.77 (m, 1H). ¹³C NMR (75 MHz, CDCl₃) δ ppm 15.3, 44.0, 48.0, 59.2, 131.0, 158.2, 161.8, 162.7, 162.8.

### Intermediate 22

### 2,4-dichloro-6-(2-chlorobenzyl)-5H-pyrrolo[3,4-d]pyrimidin-7(6H)-one

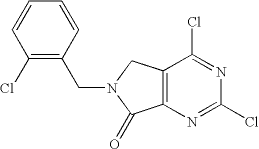

Phosphorus oxychloride (28 mL) and N,N-diethylaniline (2 mL) were added to 6-(2-Chlorobenzyl)-5,6-dihydro-1H-pyrrolo[3,4-d]pyrimidine-2,4,7(3H)-trione (Intermediate 16) (2.0 g, 6.8 mmol) and the mixture was heated to reflux for 20 min. The flask was then immediately cooled in a water bath. Phosphorus oxychloride was evaporated under reduced pressure by using toluene to ensure complete removal. Crushed ice was then added to the residue and the slurry was extracted with ethyl acetate. The organic layer was washed with brine, dried over Na₂SO₄, and concentrated under reduced pressure. The residue was triturated with dichloromethane. The insoluble starting material (1.7 g) was collected by filtration. The filtrate was concentrated under reduced pressure and the residue was purified with silica gel chromatography (ethyl acetate:hexanes 4:6) to give the title compound (0.29 g, 12%). ¹H NMR (300 MHz, CDCl₃) δ ppm 7.43-7.37 (2H, m), 7.32-7.23 (2H, m), 5.00 (2H, s), 4.39 (2H, s). ¹³C NMR (75 MHz, CDCl₃) δ ppm 162.9, 162.5, 162.3, 158.5, 134.2, 133.1, 131.4, 130.7, 130.4, 130.3, 128.1, 46.3, 45.0.

### Intermediate 23

### 2,4-dichlorofuro[3,4-d]pyrimidin-7(5H)-one

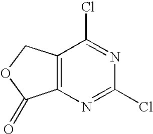

N,N-Diethylaniline (14.5 mL, 90 mmol) was added to a solution 2,4-dihydroxy-6-isopropyl-5,6-dihydropyrrolo[3,4-d]pyrimidine-7-one (intermediate 2) (10.1 g, 60 mmol) in phosphorous oxychloride (73 mL, 780 mmol) while stirring at rt. The reaction mixture was suspended in a preheated oil bath at 110° C. for 17 h. The reaction mixture was cooled to rt, concentrated under reduced pressure then triturated with ice water for 1 h. The solid was filtered to provide the title compound (12.1 g, 98%), which was used in the next step without further purification. ¹H NMR (CDCl₃) δ ppm 5.40 (s, 2H).

### Intermediate 24

### [bis(4-fluorophenyl)methyl]amine

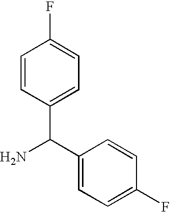

A solution of 4,4′-difluorobenzophenone (1 g, 4.6 mmol) and hydroxylamine (HCl salt) (1 g, 14.4 mmol) in 10 mL of ethanol was heated at 150° C. in a microwave for 5 minutes. The solvent was concentrated under reduced pressure and the residue was redissolved in CH₂C12. The solution was washed with water and saturated aqueous NaHCO₃, dried over MgSO₄, and evaporated to give a white solid. The material (1.05 g, 4.5 mmol) in THF (6 mL) was then added slowly to 10 mL of IM refluxing LiAlH₄ in THF. After refluxing for 4 h, the reaction was allowed to stir at rt overnight. The reaction was quenched by addition of 320 μL of water, 320 μL of 15% aqueous NaOH and then 960 μL of water, and stirred at rt for 1 h. The white solid was filtered off though diatomaceous earth and the solvent was exchanged with CH₂C12. The reaction was then worked up using acid-base extraction with 15% aqueous citric acid solution. The title compound was obtained as an oil (560 mg, 59% yield). ¹H NMR (400 MHz, CDCl₃) δ ppm 5.19 (s, 1H), 6.99 (ddd, J=8.64, 6.59, 2.15 Hz, 4H), 7.29-7.34 (m, 4H). M.S. (calcd): 220.2 (MH⁺), M.S. (found): 220.0 (ESI) (MH⁺).

### Intermediate 25

### benzyl(cyclopropylmethyl)amine

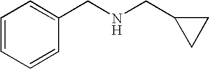

To a solution of 1-cyclopropylmethanamine (134 mg, 1.885 mmol) in methanol (10 mL) was added benzaldehyde (100 mg, 0.942 mmol) and a few drops of acetic acid. The reaction was stirred at rt for 30 minutes and then sodium cyanoborohydride (118 mg, 1.885 mmol) was added. The reaction was stirred for 3 days at rt, concentrated in vacuo, and quenched with a saturated solution of sodium bicarbonate. The aqueous layer was extracted with ethyl acetate (3×). The extracts were combined, dried (Na₂SO₄), filtered and concentrated in vacuo. The amine was obtained as an oil (35 mg, 23%) and was pure enough for the next step. M.S. (calcd): 162.3 (MH⁺), M.S. (found): 162.0 (MH⁺).

### Intermediate 26

### (4-chlorobenzyl)(cyclopropylmethyl)amine

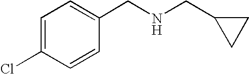

To a solution of 1-cyclopropylmethanamine (202 mg, 2.846 mmol) in methanol (15 mL) was added 4-chlorobenzaldehyde (200 mg, 1.423 mmol) and a few drops of acetic acid. The reaction was stirred at rt for 5 minutes and then sodium cyanoborohydride (179 mg, 2.846 mmol) was added. The reaction was stirred overnight at rt, concentrated in vacuo, and quenched with a saturated solution of sodium bicarbonate. The aqueous layer was extracted with ethyl acetate (3×). The extracts were combined, dried (Na₂SO₄), filtered and concentrated in vacuo. The residue was then purified by preparative LCMS (gradient 45-65% CH₃CN in H₂O containing 10 mM NH₄HCO₃). The amine was obtained as an oil (42 mg, 15%). M.S. (calcd): 196.7 (MH⁺), M.S. (found): 196.0 (MH⁺).

### Intermediate 27

### 1-cyclopropyl-N-(4-ethoxybenzyl)methenamine

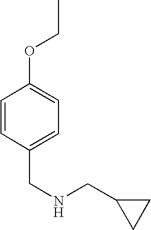

To a solution of 4-ethoxybenzaldehyde (500 mg, 3.33 mmol) in methanol (2 mL) was added cyclopropylmethanamine (237 mg, 3.33 mmol) and stirred for 10 minutes followed by addition of a solution of zinc chloride (681 mg, 4.99 mmol) and sodium cyanoborohydride (628 mg, 9.99 mmol) in methanol (2 mL) at rt. The reaction mixture was stirred for 5 h at rt, then concentrated under reduced pressure, then 50 mL DCM was added and the solution was washed with NaOH (2N aqueous solution). The organic layer was dried (MgSO₄), filtered, concentrated under reduced pressure and the residue was purified by silica gel chromatography (10-30% Methanol in DCM) to yield the title compound (660 mg, 97%). M.S. (found): 206.1 (ESI) (MHz).

### Intermediate 28

### 2-methyl-1-p-tolylpropan-2-amine hydrochloride

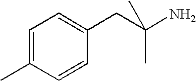

To a solution of 2-methyl-1-p-tolylpropan-2-ol (1.20 g, 7.31 mmol) in acetic acid (3 mL) was added acetonitrile (0.46 mL, 8.77 mmol) followed by conc. sulfuric acid (0.584 mL, 10.96 mmol) dropwise within 5 minutes at rt. The reaction mixture was stirred for 1 h at rt, diluted with water and brought to pH>10 using an aqueous solution of 2N NaOH. The aqueous layer was extracted with ethyl acetate (3×), the combined organic layers were dried (MgSO₄), filtered and concentrated under reduced pressure. The residue was purified by silica gel chromatography (30-60% EtOAc/Heptane) to yield N-(2-methyl-1-p-tolylpropan-2-yl)acetamide (1.28 g, 85%), which was added conc. HCl (3.79 mL, 124.70 mmol) and the solution was heated at 100° C. for 24 h. The reaction mixture was cooled to rt, water was added, and the mixture was extracted with DCM (2×). The aqueous layer was concentrated under reduced pressure to afford the title compound (0.296 g, 24%) as the HCl salt. M.S. (found): 163.9 (ESI) (MH⁺)

### Intermediate 29

### 4-ethoxy-3-fluorobenzaldehyde

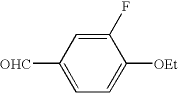

To a solution of 3-fluoro-4-hydroxybenzaldehyde (1.0 g, 7.14 mmol) in DMF (15 mL) was added K₂CO₃ (1.480 g, 10.71 mmol) followed by ethyl iodide (0.865 mL, 10.71 mmol) at rt. The reaction mixture was stirred for 18 h at 50° C. Water was added and extracted with EtOAc (2×). The organic layer was dried (MgSO₄), filtered and concentrated under reduced pressure. The residue was purified by silica gel chromatography (10-30% EtOAc in heptane) to give 4-ethoxy-3-fluorobenzaldehyde (1.100 g, 92%). ¹H NMR (400 MHz, CDCl₃) δ ppm 1.36-1.62 (m, 3H), 4.21 (q, J=7.03 Hz, 2H), 6.89-7.16 (m, 1H), 7.51-7.66 (m, 2H), 9.86 (d, J=2.34 Hz, 1H).

### Intermediate 30

### 3-chloro-4-ethoxybenzaldehyde

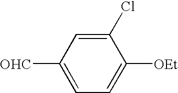

Following a procedure similar to that described for Intermediate 29 and after purification by silica gel chromatography (10-30% EtOAc in heptane), the title compound (1.05 g, 89%) was obtained. ¹H NMR (400 MHz, CDCl₃) δ ppm 1.29-1.59 (m, 3H), 4.21 (q, J=6.77 Hz, 2H), 7.02 (d, J=8.59 Hz, 1H), 7.75 (dd, J=8.40, 2.15 Hz, 1H), 7.91 (d, J=2.34 Hz, 1H), 9.85 (s, 1H).

### Intermediate 31

### 4-cyclobutoxybenzaldehyde

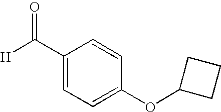

To a stirred solution of 4-hydroxybenzaldehyde (1.00 g, 8.19 mmol) in THF (40 mL) were added triphenylphosphine (3.22 g, 12.3 mmol), DEAD (4.86 mL, 12.3 mmol) and cyclobutanol (0.962 mL, 12.28 mmol). The mixture was stirred at rt for 48 h, concentrated under reduced pressure and the residue was purified by silica gel chromatography (0% to 50% EtOAc in Heptane) to give the title compound (0.282 g, 19.5%) as a yellow oil. ¹H NMR (400 MHz, CDCl₃) δ ppm 1.47-2.04 (m, 2H), 2.05-2.36 (m, 2H), 2.36-2.69 (m, 2H), 4.74 (quin, J=7.13 Hz, 1H), 6.91 (d, J=8.98 Hz, 2H), 7.82 (d, J=8.59 Hz, 2H), 9.88 (s, 1H).

### Intermediate 32

### 4-cyclopropylbenzaldehyde

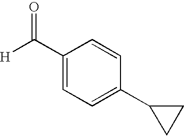

Following the procedure described in the literature (Tetrahedron Letters, 2002, 43(39), 6987-6990), the title compound (2.0 g, 87%) was obtained as an oil. ¹H NMR (400 MHz, CDCl₃) δ ppm 0.74-0.87 (m, 2H), 1.03-1.15 (m, 2H), 1.89-2.03 (m, 1H), 7.19 (d, J=8.20 Hz, 2H), 7.77 (d, J=8.20 Hz, 2H), 9.95 (s, 1H).

### Intermediate 33

### 3-fluoro-4-propoxybenzaldehyde

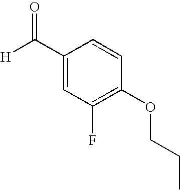

Following a procedure similar to that described for Intermediate 29, the title compound (1.93 g, 99%) was obtained as an oil. ¹H NMR (400 MHz, CDCl₃) δ ppm 0.99-1.28 (m, 3H), 1.91 (sxt, J=7.03 Hz, 2H), 3.97-4.24 (m, 2H), 7.07 (t, J=8.20 Hz, 1H), 7.50-7.75 (m, 2H), 9.86 (s, 1H).

### Intermediate 34

### 4-(cyclopropylmethoxy)-3-fluorobenzaldehyde

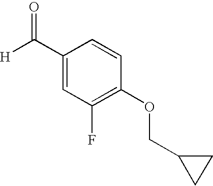

Following a procedure similar to that described for Intermediate 29, the title compound (481 mg, 99%) was obtained as an oil. ¹H NMR (400 MHz, CDCl₃) δ ppm 0.33-0.47 (m, 2H), 0.62-0.80 (m, 2H), 1.22-1.44 (m, 1H), 3.98 (d, J=7.03 Hz, 2H), 7.05 (t, J=8.01 Hz, 1H), 7.62 (d, J=9.38 Hz, 2H), 9.86 (d, J=1.95 Hz, 1H).

### Intermediate 35

### 4-(hydroxymethyl)benzaldehyde

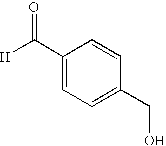

To (4-(dimethoxymethyl)phenyl)methanol (1.50 g, 8.23 mmol) in THF (3 mL) was added a 3% aqueous solution of H₂SO₄ (3 mL). The mixture was stirred at rt for 3 h, then saturated aqueous solution of NaHCO₃ (10 mL) was added and the mixture was extracted with EtOAc (3×), washed with brine, dried (MgSO₄), filtered and concentrated under reduced pressure to give the title compound (1.17 g, 104%) as a solid. ¹H NMR (400 MHz, CDCl₃) δ ppm 2.10 (br. s., 1H), 4.81 (d, J=4.69 Hz, 2H), 7.53 (d, J=7.81 Hz, 2H), 7.88 (d, J=7.81 Hz, 2H), 10.00 (s, 1H).

### Intermediate 36

### 4-(methoxymethyl)benzaldehyde

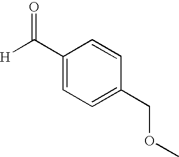

To a mixture of sodium hydride (220 mg, 5.51 mmol) in THF (6 mL) was added a solution of 4-(hydroxymethyl)benzaldehyde (500 mg, 3.67 mmol) in THF (6.00 mL) at rt, the reaction mixture was stirred for 15 minutes. Iodomethane (0.344 mL, 5.51 mmol) was added and the reaction mixture was stirred at rt for 16 h. Water was added and the mixture was extracted with EtOAc (3×), washed with brine, dried (MgSO₄), filtered and concentrated under reduced pressure. The residue was purified by silica gel chromatography (0-50% EtOAc in heptane) to give the title compound (195 mg, 35.4%) as an oil. ¹H NMR (400 MHz, CDCl₃) δ ppm 3.32-3.39 (m, 9H), 3.41 (s, 3H), 4.43 (s, 7H), 4.52 (s, 2H), 7.47 (d, J=6.64 Hz, 3H), 7.85 (d, J=6.64 Hz, 2H), 9.99 (s, 1H).

### Intermediate 37

### 4-ethoxy-2-methoxybenzaldehyde

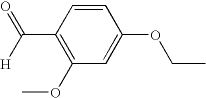

Following a procedure similar to that described for Intermediate 29, the title compound (1.14 g, 96%) was obtained and was used without further purification. ¹H NMR (400 MHz, CDCl₃) δ ppm 1.45 (t, J=7.03 Hz, 3H), 3.90 (s, 3H), 4.11 (q, J=7.03 Hz, 2H), 6.44 (d, J=1.95 Hz, 1H), 6.53 (dd, J=8.79, 2.15 Hz, 1H), 7.80 (d, J=8.59 Hz, 1H), 10.28 (s, 1H).

### Intermediate 38

### 4-isopropoxy-2-methoxybenzaldehyde

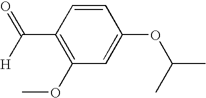

Following a procedure similar to that described for Intermediate 29 and after purification by silica gel chromatography (10-30% EtOAc in heptane), the title compound (1.20 g, 94%) was obtained as an oil. ¹H NMR (400 MHz, CD₃OD) δ ppm 1.38 (d, J=6.25 Hz, 6H), 3.89 (s, 3H), 4.66 (quin, J=6.05 Hz, 1H), 6.43 (d, J=2.34 Hz, 1H), 6.52 (dd, J=8.59, 1.95 Hz, 1H), 7.79 (d, J=8.98 Hz, 1H), 10.27 (s, 1H).

### Intermediate 39

### 4-ethoxy-2-fluorobenzaldehyde

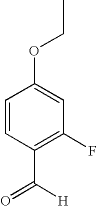

Following a procedure similar to that described for Intermediate 29, the title compound (1.20 g, 100%) was obtained and was used without further purification. ¹H NMR (400 MHz, CD₃OD) δ ppm 1.45 (t, J=7.03 Hz, 3H), 4.10 (q, J=6.77 Hz, 2H), 6.62 (dd, J=12.50, 2.34 Hz, 1H), 6.77 (dd, J=8.79, 2.15 Hz, 1H), 7.75-7.85 (m, 1H), 10.20 (s, 1H).

### Intermediate 40

### (R)-1-(4-ethoxy-3-fluorophenyl)ethanamine hydrochloride

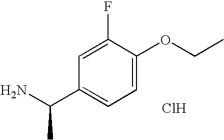

To a solution of (S)-2-methylpropane-2-sulfinamide (375 mg, 3.09 mmol) in DCM (15 mL) was added 4-ethoxy-3-fluorobenzaldehyde (Intermediate 29) (623 mg, 3.71 mmol) followed by pyridinium p-toluenesulfonate (78 mg, 0.31 mmol) and MgSO₄ (1.86 g, 15.47 mmol) at rt. The reaction mixture was stirred for 18 h at 40° C., after filtered and concentrated under reduced pressure, the residue was purified by silica gel chromatography (5-25% EtOAc in heptane) to give (S,E)-N-(4-ethoxy-3-fluorobenzylidene)-2-methylpropane-2-sulfinamide (570 mg, 67.9%). [α]_{D}+12.5 (c=0.1, MeOH). ¹H NMR (400 MHz, CDCl₃) δ ppm 1.26 (s, H), 1.50 (t, J=7.03 Hz, 3H), 4.18 (q, J=7.03 Hz, 2H), 7.01 (t, J=8.20 Hz, 1H), 7.50 (d, J=8.98 Hz, 1H), 7.66 (dd, J=11.72, 1.95 Hz, 1H), 8.47 (d, J=1.56 Hz, 1H). To a solution of the above intermediate (530 mg, 1.95 mmol) in tetrahydrofuran (10 mL) was added a solution of methylmagnesium bromide (9.77 mL, 9.77 mmol) in butyl ether at −30° C. The reaction mixture was stirred for 18 h at rt. Water was added and extracted with EtOAc (3×). The combined organic phases were dried (MgSO₄), filtered and concentrated under reduced pressure. The residue was purified by silica gel chromatography (100% EtOAc to 40% MeOH in Et)Ac) to give (S)—N—((R)-1-(4-ethoxy-3-fluorophenyl)ethyl)-2-methylpropane-2-sulfinamide (550 mg, 98%). [α]D+96.9 (c=0.1, MeOH). M.S. (found): 288.1 (ESI) (MH⁺). To a solution of the above intermediate (541 mg, 1.88 mmol) in methanol (2 mL) was added a solution of hydrogen chloride (2.349 mL, 9.39 mmol) in 1,4-dioxane at rt. The reaction mixture was stirred for 2 h at rt. After concentration under reduced pressure, Et₂O was added and the solid was collected by filtration to give the title compound (412 mg, 100%) as a hydrochloride salt. ¹H NMR (400 MHz, CD₃OD) δppm 1.39 (t, J=7.03 Hz, 2H), 1.57 (d, J=7.03 Hz, 3H), 4.10 (q, J=7.03 Hz, 2H), 4.38 (q, J=6.64 Hz, 1H), 7.06-7.26 (m, 3H). [α]_{D}=+6.06 (c=0.01, MeOH).

The following intermediates (Intermediate 41-57) were prepared by using a procedure similar to that described for the preparation of Intermediate 40:

| Intermediate | | | |
|---|---|---|---|
| # | Structure | Name | Analytical Data |
| 41 | 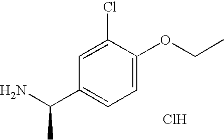 | (R)-1-(3-chloro-4-ethoxyphenyl)ethanaminehydrochloride | ¹H NMR (400 MHz, CD₃OD) δ ppm1.41 (t, J = 7.03 Hz, 3 H), 1.58 (d, J =6.64 Hz, 3 H), 4.11 (q, J = 7.03 Hz,2 H), 4.38 (q, J = 6.90 Hz, 1 H), 7.09(d, J = 8.59 Hz, 1 H), 7.33 (dd, J =8.59, 2.34 Hz, 1 H), 7.48 (d, J = 2.34Hz, 1 H). [α]_{D} = +8.2 (c = 0.013,MeOH). |
| 42 | 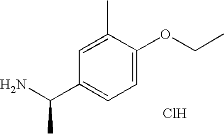 | (R)-1-(4-ethoxy-3-methylphenyl)ethanaminehydrochloride | ¹H NMR (400 MHz, DMSO-d₆) δppm 1.32 (t, J = 6.84 Hz, 3 H), 1.46(d, J = 6.64 Hz, 3 H), 2.14 (s, 3 H),4.02 (q, J = 7.03 Hz, 2 H), 4.26 (s,1 H), 6.94 (d, J = 9.37 Hz, 1 H), 7.21-7.35 (m, 2 H), 8.35 (s, 2 H). [α]_{D} =+7.9 (c = 0.01, MeOH). |
| 43 | 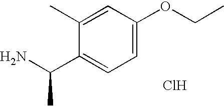 | (R)-1-(4-ethoxy-2-methylphenyl)ethanaminehydrochloride | ¹H NMR (400 MHz, CDCl₃) δ ppm1.36 (t, J = 7.03 Hz, 3 H), 1.57 (d, J =6.64 Hz, 3 H), 2.30 (s, 3 H), 3.96(q, J = 7.03 Hz, 2 H), 4.55 (s, 1 H),6.65 (d, J = 2.34 Hz, 1 H), 6.71 (dd, J =8.59, 2.34 Hz, 1 H), 7.49 (d, J =8.59 Hz, 1 H), 8.57 (s, 2 H). [α]_{D} =+7.2 (c = 0.01, MeOH). |
| 44 | 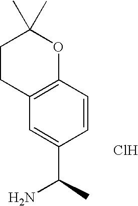 | (R)-1-(2,2-dimethylchoman-6-yl)-ethanaminehydrochloride | ¹H NMR (400 MHz, CDCl₃) δ ppm1.18 (s, 6 H), 1.49 (d, J = 6.64 Hz, 3 H), 1.67 (t, J = 6.64 Hz, 2 H), 2.65(t, J = 6.64 Hz, 2 H), 4.14 (s, 1 H), 6.64 (d, J = 8.20 Hz, 1 H), 6.99 (s, 2 H). [α]_{D} = +7.5 (c = 0.01, MeOH). |
| 45 | 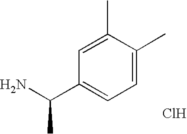 | (R)-1-(3,4-dimethylphenyl)ethanaminehydrochloride | ¹H NMR (400 MHz, CDCl₃) δ ppm1.60 (d, J = 6.64 Hz, 3 H), 1.82 (s, 2 H), 2.20 (s, 6 H), 4.26 (s, 1 H), 7.07(d, J = 7.42 Hz, 1 H), 7.14-7.22 (m,2 H). [α]_{D} = +7.0 (c = 0.01, MeOH). |
| 46 | 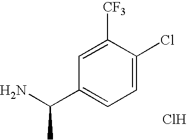 | (R)-1-(4-chloro-3-(trifluoromethyl)phenyl)ethanaminehydrochloride | ¹H NMR (400 MHz, CD₃OD) δ ppm1.65 (d, J = 6.64 Hz, 3 H), 4.59 (d, J =6.64 Hz, 1 H), 7.73 (s, 2 H), 7.92 (s, 1 H). [α]_{D} = +1.5 (c = 0.01, MeOH). |
| 47 | 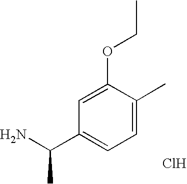 | (R)-1-(3-ethoxy-4-methylphenyl)ethanaminehydrochloride | ¹H NMR (400 MHz, CDCl₃) δ ppm1.28 (t, J = 6.84 Hz, 3 H), 1.60 (d, J =7.03 Hz, 3 H), 2.17 (s, 3 H), 3.68-4.01 (m, 2 H), 4.20-4.34 (m, 1 H),6.80 (d, J = 7.42 Hz, 1 H), 6.98 (s, 1 H), 7.04 (d, J = 7.42 Hz, 1 H), 8.68(s, 2 H). [α]_{D} = +7.5 (c = 0.01, MeOH). |
| 48 | 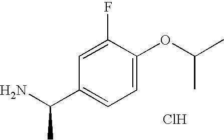 | (R)-1-(3-fluoro-4-isopropoxyphenyl)ethanaminehydrochloride | ¹H NMR (400 MHz, DMSO-d6) δppm 1.27 (d, J = 5.86 Hz, 6 H), 1.48(d, J = 7.03 Hz, 3 H), 4.34 (d, J =5.86 Hz, 1 H), 4.65 (dt, J = 12.11,6.05 Hz, 1 H), 7.13-7.32 (m, 2 H),7.36-7.54 (m, 1 = H), 8.52 (br. s.,2 H). [α]_{D} = +6.0 (c = 0.01, MeOH). |
| 49 | 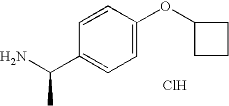 | (R)-1-(4-cyclobutoxyphenyl)ethanamine hydrochloride | ¹H NMR (400 MHz, DMSO-d₆) δppm 1.46 (d, J = 7.03 Hz, 3 H), 1.55-1.87 (m, 2 H), 2.00 (t, J = 9.77 Hz, 2 H), 2.31-2.47 (m, 2 H), 4.34 (br. s.,1 H), 4.70 (t, J = 7.23 Hz, 1 H), 6.89(d, J = 8.59 Hz, 2 H), 7.37 (d, J =8.59 Hz, 2 H), 8.16 (br. s., 2 H).[α]_{D} = +7.3 (c = 0.01, MeOH). |
| 50 | 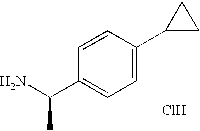 | (R)-1-(4-cyclopropylphenyl)ethanaminehydrochloride | ¹H NMR (400 MHz, DMSO-d₆) δppm 0.57-0.73 (m, 2 H), 0.87-1.02(m, 2 H), 1.48 (d, J = 6.64 Hz, 3 H),1.91 (spt, 1 H), 4.31 (t, 1 H), 7.11 (d, J = 8.20 Hz, 2 H), 7.38 (d, J = 8.20Hz, 2 H), 8.53 (br. s., 2 H). [α]_{D} =+7.7 (c = 0.01, MeOH). |
| 51 | 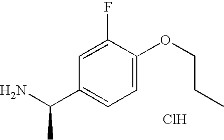 | (R)-1-(3-fluoro-4-propoxyphenyl)ethanaminehydrochloride | ¹H NMR (400 MHz, DMSO-d₆) δppm 0.97 (t, J = 7.23 Hz, 3 H), 1.49(d, J = 6.64 Hz, 3 H), 1.73 (sxt, J =7.03 Hz, 2 H), 4.01 (t, J = 6.64 Hz,2 H), 4.23-4.46 (m, 1 H), 7.19 (t, J =8.59 Hz, 1 H), 7.24-7.34 (m, 1 H),7.46 (dd, J = 12.50, 1.95 Hz, 1 H),8.59 (br. s., 2 H). [α]_{D} = +4.4(c = 0.01, MeOH). |
| 52 | 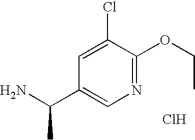 | (R)-1-(5-chloro-6-ethoxypyridin-3-yl)ethanaminehydrochloride | ¹H NMR (400 MHz, DMSO-d₆) δppm 1.34 (t, J = 7.03 Hz, 3 H), 1.53(d, J = 6.64 Hz, 3 H), 4.15-4.63 (m,3 H), 8.16 (d, J = 1.95 Hz, 1 H), 8.25(d, J = 1.56 Hz, 1 H), 8.63 (br. s.,2 H). [α]_{D} = +5.0 (c = 0.01, MeOH). |
| 53 | 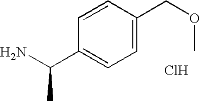 | (R)-1-(4-(methoxymethyl)phenyl)ethanaminehydrochloride | ¹H NMR (400 MHz, CD₃OD) δ ppm1.56 (d, J = 7.03 Hz, 3 H), 3.20-3.28 (m, 1 H), 4.40 (s, 2 H), 4.81 (s, 3 H), 7.37 (d, J = 1.95 Hz, 4 H).[α]_{D} = +3.0 (c = 0.01, MeOH). |
| 54 | 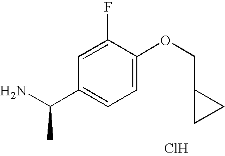 | (R)-1-(4-(cyclopropylmethoxy)-3-fluorophenyl)ethanaminehydrochloride | ¹H NMR (400 MHz, CD₃OD) δ ppm0.27-0.44 (m, 2 H), 0.63 (q, J = 5.86Hz, 2 H), 1.18 (s, 2 H), 1.23-1.36(m, 1 H), 1.60 (d, J = 6.64 Hz, 3 H),3.91 (d, J = 7.03 Hz, 2 H), 4.41 (q, J =6.90 Hz, 1 H), 7.02-7.34 (m, 3 H).[α]_{D} = +5.8 (c = 0.01, MeOH). |
| 55 | 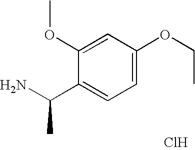 | (R)-1-(4-ethoxy-2-methoxyphenyl)ethanaminehydrochloride | ¹H NMR (400 MHz, DMSO-d₆) δppm 1.32 (t, J = 7.03 Hz, 3 H), 1.45(d, J = 7.03 Hz, 3 H), 3.81 (s, 3 H),4.04 (q, 2 H), 4.47 (qt, 1 H), 6.56 (dd,1 H), 6.59 (d, 1 H), 7.35 (d, 1 H). |
| 56 | 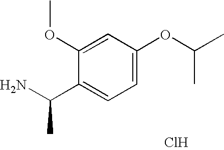 | (R)-1-(4-isopropoxy-2-methoxyphenyl)ethanaminehydrochloride | ¹H NMR (400 MHz, DMSO-d₆) δppm 1.21 (d, J = 5.86 Hz, 6 H), 1.40(d, J = 7.03 Hz, 3 H), 3.75 (s, 3 H),4.48 (m, 1 H), 4.61 (quin, J = 6.05Hz, 1 H), 6.49-6.55 (m, 2 H), 7.24-7.33 (m, 1 H). |
| 57 | 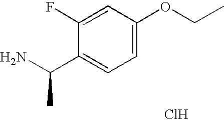 | (R)-1-(4-ethoxy-2-fluorophenyl)ethanaminehydrochloride | ¹H NMR (400 MHz, DMSO-d₆) δppm 1.42 (br. s., 3 H), 1.70 (br. s.,3 H), 4.01 (br. s., 2 H), 4.74 (br. s.,1 H), 6.51-6.78 (m, 2 H), 7.53 (br. s.,1 H). |

### Intermediate 58

### 1-(2-methoxyphenyl)-2-methylpropan-2-amine

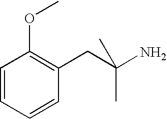

To a 1.4M solution of methylmagnesium bromide (11.31 mL, 15.83 mmol) in toluene/THF (75/25) was added drop-wise a solution of 1-(2-methoxyphenyl)propan-2-one (1.898 mL, 12.18 mmol) dissolved in diethyl ether (20 mL) at 0° C. The mixture was allowed to warm up to rt and stirred for 90 minutes. Saturated ammonium chloride solution was added and the mixture was extracted with ethyl ether (×3). The combined organic phases were dried (Na₂SO₄), filtered and concentrated under reduced pressure. The residue was purified by silica gel chromatography (1-10% MeOH in DCM) to give 1-(2-methoxyphenyl)-2-methylpropan-2-ol (1.12 g, 51.0%). ¹H NMR (400 MHz, CDCl₃) δ ppm 1.21 (s, 6H), 2.86 (s, 2H), 3.83 (s, 3H), 6.91 (qd, J=7.81, 7.52, 0.98 Hz, 2H), 7.13 (dd, J=7.42, 1.95 Hz, 1H), 7.20-7.26 (m, 1H). To a solution of the above intermediate and acetonitrile (0.452 mL, 8.59 mmol) in acetic acid (5 mL) was added conc. sulfuric acid (0.572 mL, 10.74 mmol) dropwise within 5 minutes at rt. The reaction mixture was stirred for 1 h, diluted with water, brought to pH>10 with 2N NaOH solution and extracted with ethyl acetate (2×). The combined organic phases were dried (Na₂SO₄), filtered and concentrated under reduced pressure. The residue was purified by silica gel chromatography (0-10% MeOH in DCM) to give N-(1-(2-methoxyphenyl)-2-methylpropan-2-yl)acetamide (1.12 g, 70.7%). M.S. 222.01 (ESI) (MH⁺).

To a solution of the above intermediate (1.12 g, 5.06 mmol) in ethylene glycol (11.32 mL) was added potassium hydroxide (0.568 g, 10.12 mmol). The reaction mixture was heated in a microwave reactor at 230° C. for 2 h, cooled to rt, combined with ice water and extracted with diethyl ether (3×). The organic phase was treated with HCl 10%. The aqueous phase was washed with diethyl ether (3×), brought to pH>10 with concentrated KOH and extracted with DCM (3×). The combined organic phases were dried (Na₂SO₄), filtered and concentrated under reduced pressure to give the title compound (0.588 g, 64.8%), which was used in the next step without further purification. M.S. 179.98 (ESI) (MH⁺).

### Intermediate 59

### 1-(4-methoxyphenyl)-2-methylpropan-2-amine

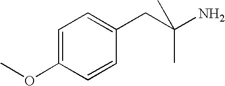

Following a procedure similar to that described in Intermediate 58 and after purification by preparative HPLC (gradient 20-40% CH₃CN in H₂O containing 10 mM NH₄HCO₃), the title compound was obtained (0.280 g, 14% over 3 steps). MS [M+H]⁺ 179.97 (ESI).

### Intermediate 60

### 2-methyl-1-o-tolylpropan-2-amine

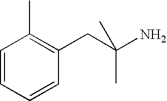

Following a procedure similar to that described in Intermediate 58, the title compound was obtained (0.214 g, 10% over 3 steps), which was used in the next step without further purification. MS [M+H]⁺ 163.94 (ESI).

### Intermediate 61

### 1-(4-ethoxyphenyl)-2-methylpropan-2-amine

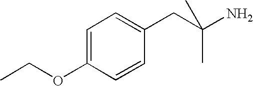

Following a procedure similar to that described in Intermediate 58, the title compound was obtained (0.185 g, 8.7% over 3 steps), which was used in the next step without further purification. MS [M+H]⁺ 193.98 (ESI).

### Intermediate 62A

### 1-(quinolin-3-yl)ethanamine

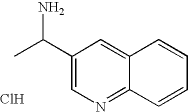

To a solution of 2-methyl propane-2-sulfinamide (1.490 g, 12.09 mmol) in dichloromethane (50 mL) was added quinoline-3-carbaldehyde (2.000 g, 12.73 mmol), pyridinium 4-methylbenzenesulfonate (0.160 g, 0.64 mmol), and magnesium sulfate (20 g). The reaction was stirred at rt for 48 h. An additional 10 g of magnesium sulfate was added after 48 h. And stirring was continued for an additional 12 h. After filtration and concentration under reduced pressure, the residue was dissolved in THF (2 mL) and methylmagnesium bromide (0.274 mL, 0.38 mmol) was added dropwise to the above solution at −78° C. The reaction mixture was stirred at −78° C. for 2 h, then slowly warmed to rt and stirred for 16 h. Ice-water was added and the mixture was extracted with ethyl acetate. The organic phase was washed with water and brine. After the removal of solvent under reduced pressure, the residue was purified by silica gel column (0-100% ethyl acetate/heptane) to give 2-methyl-N-(1-(quinolin-3-yl)ethyl)propane-2-sulfinamide (42 mg, 79%). ¹H NMR (400 MHz, DMSO-d₆) δ ppm 1.09 (s, 3H), 1.61 (d, J=7.0 Hz, 3H), 5.10-5.25 (m, 1H), 5.50 (d, J=5.9 Hz, 1H), 7.55-7.45 (m, 2H), 7.60 (d, J=6.6 Hz, 1H), 7.81 (d, J=8.2 Hz, 1H), 7.90-7.95 (m, 1H), 8.78 (d, J=8.2 Hz, 1H). To the above intermediate in 1,4-dioxane (5 mL) and MeOH (5.0 mL) was added hydrogen chloride (0.904 mL, 3.62 mmol). The reaction mixture was stirred at rt for 1 h, then concentrated under reduced pressure to give the title compound, which was used in the next step without further purification.

### Intermediate 62B

### N-(isoquinoline-3-ylmethyl)ethanamine

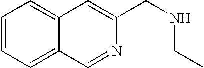

To a solution of tert-butyl isoquinolin-3-ylmethylcarbamate (300 mg, 1.16 mmol) in THF (20 mL) was added sodium hydride (93 mg, 2.32 mmol) followed by iodoethane (362 mg, 2.32 mmol) at −78° C. The reaction was stirred at −78° C. for 30 minutes, allowed to warm up to rt and stirred for 16 h. Water was added and the mixture was extracted with ethyl acetate, washed with water and brine. The organic layer was dried (MgSO₄), filtered and concentrated under reduced pressure. The residue was purified with silica gel chromatography (25-50% acetyl acetate/heptane) to give tert-butyl ethyl(isoquinolin-3-ylmethyl)carbamate (237 mg, 71.6%) as an oil. M.S. (calcd): 287.38 (MH⁺), M.S. (found): 287.24 (ESI) (MH⁺). To a solution of the above intermediate (150 mg, 0.52 mmol) in dichloromethane (2 mL) was added TFA (1 mL). The reaction mixture was stirred at 40° C. for 30 minutes. The solvent was removed under reduced pressure to give the title compound as its TFA salt, which was used in the next step reaction without further purification. M.S. (calcd): 187.25 (MH⁺), M.S. (found): 186.99 (ESI) (MH⁺).

### Intermediate 63

### N-(quinolin-3-ylmethyl)ethanamine

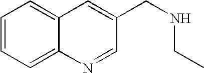

To a solution of quinolin-3-ylmethanamine (500 mg, 3.16 mmol), di-tert-butyl dicarbonate (828 mg, 3.79 mmol) in 1 mL of DIPEA and EtOH (10 mL) was stirred at rt for 2 h. The solvent was removed under reduced pressure and the residue was purified by silica gel chromatography (eluted with 25-50% ethyl acetate/heptane) to give tert-butyl quinolin-3-ylmethylcarbamate (700 mg, 86%) as a solid. M.S. (calcd): 259.36 (MH⁺), M.S. (found): 259.14 (ESI) (MH⁺).

To a solution of the above intermediate (300 mg, 1.16 mmol) and sodium hydride (93 mg, 2.32 mmol) in THF (20 mL) was added iodoethane (362 mg, 2.32 mmol) at −78° C. The reaction was stirred at −78° C. for 30 minutes, allowed to warm up to rt and stirred for 16 h. Water was added and the mixture was extracted with ethyl acetate, washed with water and brine. The organic layer was dried (MgSO₄), filtered and concentrated under reduced pressure. The residue was purified with silica gel chromatography (25-50% acetyl acetate/heptane) to give tert-butyl ethyl(quinolin-3-ylmethyl)carbamate (230 mg, 69.2%) as an oil. M.S. (calcd): 287.37 (MH⁺), M.S. (found): 287.24 (ESI) (MH⁺). A solution of the above intermediate (150 mg, 0.52 mmol) in CH₂Cl₂ (2 mL) and 1 mL of TFA was stirred at 40° C. for 1 h. The solvent was removed under reduced pressure to give the title compound as its TFA salt, which was used in the next step without further purification. M.S. (calcd): 187.25 (MH⁺), M.S. (found): 187.24 (ESI) (MH⁺).

### Intermediate 64

### (2-methylquinolin-3-yl)methenamine

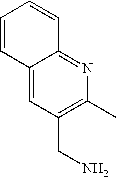

A solution of ethyl 2-methylquinoline-3-carboxylate (2.35 g, 10.92 mmol) and lithium aluminum hydride (0.829 g, 21.84 mmol) in THF (10 mL) was stirred at rt for 2 h. The reaction was quenched with Na₂SO₄.5H₂O, extracted with ethyl acetate (3×). The combined organic phases were dried (Na₂SO₄), filtered and concentrated under reduced pressure. The residue was purified by silica gel chromatography (25-50% ethyl acetate/heptane) to give (2-methylquinolin-3-yl)methanol (1.34 g, 71%) as a solid. M.S. (calcd): 174.21 (MH⁺), M.S. (found): 174.18 (ESI) (MH⁺). To a solution of the above intermediate (0.720 g, 4.16 mmol) in anhydrous CH₂Cl₂ (20 mL) was added SOCl₂ (1.484 g, 12.47 mmol) at 0° C. The reaction mixture was stirred at 0° C. for 1 h. The solvent was removed under reduced pressure to give (2-methylquinolin-3-yl)methanyl chloride as an oil, which was used in the next step without further purification. M.S. (calcd): 192.66 (MH⁺), M.S. (found): 192.14 (ESI) (MH⁺). The above crude intermediate (0.78 g, 4.16 mmol) was dissolved in DMF (5 mL), then sodium azide (0.540 g, 8.31 mmol) and KI (10 mg) were added. The reaction was stirred at rt for 16 h. Water was added and the mixture was extracted with ethyl acetate, washed with water and brine. The organic phase was dried (Na₂SO₄), filtered and concentrated under reduced pressure to give (2-methylquinolin-3-yl)methanyl azide as an oil, which was used in the next step without further purification. M.S. (calcd): 199.23 (MH⁺), M.S. (found): 199.18 (ESI) (MH⁺). The crude (2-methylquinolin-3-yl)methanyl azide (4.16 mmol) was dissolved in THF and was added triphenyl phosphine (2.181 g, 8.31 mmol). The reaction mixture was stirred at rt for 8 h and concentrated under reduced pressure. The residue was purified by silica gel chromatography (50% ethyl acetate/heptane) to give the title compound (0.36 g, 50.3% over 3 steps) as a solid. M.S. (calcd): 172.23 (MH⁺), M.S. (found): 172.3 (ESI) (MH⁺).

### Intermediate 65

### N-methyl-1-(quinolin-2-yl)methanamine

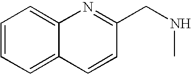

To a solution of quinolin-2-ylmethanamine (1.0 g, 6.32 mmol) in ethanol (10 mL) and 10% NaHCO₃ (2 mL) was added di-tert-butyl dicarbonate (1.38 g, 6.32 mmol). The reaction mixture was stirred at rt for 2 h, extracted with ether and washed with water and brine. The organic phase was concentrated under reduced pressure. The residue was dissolved in anhydrous THF (10 mL), sodium hydride (506 mg, 12.64 mmol) was added at 0° C. The reaction was stirred at 0° C. for 5 minutes, then iodomethane (4.49 g, 31.61 mmol) was added. The reaction mixture was stirred at rt for 2 h. Water (2 mL) was added, and the mixture was extracted with ether (2×) and washed with water and brine, The organic phase was concentrated under reduced pressure, the residue was treated with a solution of 10% HCl in methanol (20 mL) at rt for 30 minutes. The reaction mixture was concentrated under reduced pressure. The product was purified by silica gel chromatography (0-20% methanol/dichloromethane with 0.5% DIPEA). The title compound (350 mg, 32.1%) was obtained as a solid. M.S. (calcd): 173.23 (MH⁺), M.S. (found): 172.96. (ESI) (MH⁺).

### Intermediate 66

### 3-(aminomethyl)-N,N-dimethylisoquinolin-1-amine

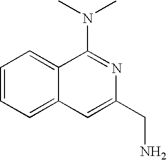

A solution of 1,3-dichloroisoquinoline (1.0 g, 5.05 mmol) and dimethylamine (0.25 g, 5.55 mmol) in BuOH (15 mL) was heated at 100° C. in a microwave reactor for 1 h. The solvent was removed under reduced pressure, the residue was dissolved in ethyl acetate and washed with water and brine. The dried organic phase was concentrated under reduced pressure and the residue was combined with a mixture of diphenylphosphino ferrocene (dppf) (0.095 g, 0.10 mmol), tris(dibenzylideneacetone) dipalladium (0) (137 mg, 0.15 mmol), Zn(CN)₂ (0.352 g, 3.00 mmol), zinc powder (6.54 mg, 0.10 mmol) and 3-chloro-N,N-dimethylisoquinolin-1-amine (1.033 g, 5 mmol) in DME (15 mL) and water (0.3 mL). The reaction mixture was heated at 130° C. in a microwave reactor for 15 minutes, cooled to rt and filtered though diatomaceous earth and concentrated under reduced pressure. The residue was purified by silica gel chromatography (eluted with 0-40% ethyl acetate/heptane) to give 1-(dimethylamino)isoquinoline-3-carbonitrile (0.56 g, 56.8%) as a solid. M.S. (calcd): 517.58 (MH⁺), M.S. (found): 517.3. (ESI) (MH⁺). To a mixture of the above intermediate in MeOH (15 mL) was added 10 M aqueous hydrogen chloride (1.42 mL, 14.20 mmol). The reaction was stirred at 100° C. for 1 h, cooled to rt, concentrated under reduced pressure. The residue was dissolved in THF (15 mL) and lithium aluminum hydride (108 mg, 2.84 mmol) was added at 0° C. and then stirred at 0° C. for 2 h. The excess hydride was quenched by adding 20% aqueous NaOH and the mixture was extracted with ethyl acetate. The combined organic phases were washed with water and brine, dried over Na₂SO₄, filtered and concentrated under reduced pressure to give [1-(dimethylamino)-3-yl]methanol, which was used in the next step without further purification. M.S. (calcd): 204.27 (MH⁺), M.S. (found): 204.5. (ESI) (MH⁺). To a solution of [1-(dimethylamino)-3-yl]methanol in CH₂Cl₂ (15.00 mL) was added SOCl₂ (338 mg, 2.84 mmol) at 0° C. The reaction was stirred at 0° C. for 10 minutes, warmed to rt and stirred at rt for an additional 30 minutes. The reaction mixture was concentrated under reduced pressure, the residue was dissolved in DMF (15.00 mL) and sodium azide (203 mg, 3.12 mmol) was added at rt. The reaction mixture was stirred at rt for 1 h. Water was added and the mixture was extracted with ethyl acetate, washed with NaHSO₃, water and brine, dried over Na₂SO₄, filtered and concentrated under reduced pressure. The residue was dissolved in THF (10 mL) and triphenylphosphine (745 mg, 2.84 mmol) was added, the reaction mixture was stirred at rt for 12 h. After concentration under reduced pressure. The residue was purified by silica gel chromatography (eluted with 2:2:1 ethyl acetate/heptane/methanol) to give the title compound (282 mg, 49.3%). M.S. (calcd): 202.27 (MH⁺), M.S. (found): 201.94. (ESI).

### Intermediate 67

### 1-cyclopropyl-N-(isoquinolin-3-ylmethyl)methenamine

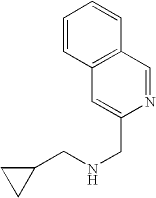

A mixture of isoquinolin-3-ylmethanamine (451 mg, 2.85 mmol), sodium triacetoxyhydroborate (242 mg, 1.14 mmol) and cyclopropanecarbaldehyde (80 mg, 1.14 mmol) in CH₂Cl₂ (10 mL) was stirred at 0° C. for 30 minutes. The reaction mixture was concentrated under reduced pressure. The residue was purified by silica gel chromatography (2:2:1 ethyl acetate/heptane/methanol) to give the title compound (37.3 mg, 14.9%) as a gum. M.S. (calcd): 213.29 (MH⁺), M.S. (found): 213.00. (ESI) (MH⁺).

### Intermediate 68

### 3-(chloromethyl)isoquinoline

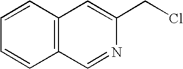

To a solution of methyl isoquinoline-3-carboxylate (5.00 g, 26.71 mmol) at 0° C. was added lithium aluminum hydride (1.014 g, 26.71 mmol) portion-wise. The reaction was stirred at 0° C. for 30 mixtures, quenched with 20% NaOH and extracted with ethyl acetate, washed with water and brine, dried over Na₂SO₄ and filtered. The filtrate was concentrated under reduced pressure to give isoquinolin-3-yl methanol as a solid, which was used in the next step without further purification. M.S. (calcd): 160.18 (MH⁺), M.S. (found): 159.95. A solution of the above isoquinolin-3-ylmethanol (2.0 g, 12.56 mmol) and sulfurous dichloride (1.495 g, 12.56 mmol) CH₂Cl₂ (25 mL) was stirred at 0° C. for 30 minutes. The solvent was removed under reduced pressure to give the title compound as an oil, which was used in the next step without further purification. M.S. (calcd): 178.63 (MH⁺), M.S. (found): 179.01.

### Intermediate 69

### N-(isoquinolin-3-ylmethyl)propan-2-amine

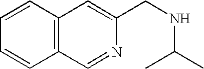

To a solution of propan-2-amine (2.0 g, 33.9 mmol) in 10 mL of ethanol was added 3-(chloromethyl)isoquinoline (Intermediate 68) (200 mg, 1.13 mmol). The reaction mixture was stirred at 80° C. for 15 minutes. After concentration under reduced pressure, the residue was purified by silica gel chromatography (2:2:0.2 EtOAc/heptane/methanol) to give the title compound (101 mg, 44.8% over 3 steps) as a gum. M.S. (calcd): 201.28 (MH⁺), M.S. (found): 201.04 (ESI) (MH⁺).

### Intermediate 70

### 1-(isoquinolin-3-yl)-N-methylmethanamine

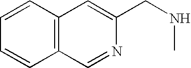

A solution of 3-(chloromethyl)isoquinoline (Intermediate 68) (100 mg, 0.56 mmol) in 10 mL of dichloromethane was added dropwise to the aqueous solution of methyl amine at 0° C. The reaction was stirred at 0° C. for 30 minutes and warmed to rt. The solvent was removed under reduced pressure. The residue was purified by silica gel chromatography (50% ethyl acetate/methanol) to give the title compound (75 mg, 77% over 3 steps) as an oil. M.S. (calcd): 173.23 (MH⁺), M.S. (found): 172.90 (ESI) (MH⁺).

### Intermediate 71

### 2,2-difluoro-N-(isoquinolin-3-ylmethyl)ethanamine

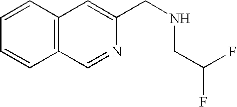

A mixture of isoquinoline-3-carbaldehyde (150 mg, 0.95 mmol), 2,2-difluoroethanamine (77 mg, 0.95 mmol) and sodium triacetoxyhydroborate (405 mg, 1.91 mmol) in CH₂Cl₂ (3 mL) was stirred at rt for 1 h. The solvent was removed under reduced pressure, the residue was purified by silica gel chromatography (ethyl acetate/heptane/methanol 2/2/0.1) to give the title compound (100 mg, 47.1%) as an oil. M.S. 222.9 (ESI) (MH⁺).

### Intermediate 72

### (1-methylisoquinolin-3-yl)methenamine

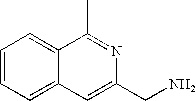

A solution of tetrakis(triphenylphosphine)palladium(0) (875 mg, 0.76 mmol), 1,3-dichloroisoquinoline (3.0 g, 15.15 mmol) and methylzinc(II) chloride (1.76 g, 15.15 mmol) in THF (20 mL) was stirred at 60° C. for 1 h in a microwave reactor (note: the reactions were performed in 3 batches with 1 g of 1,3-dichloroisoquinoline each time). After cooled to rt and concentrated under reduced pressure, the residue was purified by silica gel chromatography (10% ethyl acetate/heptane) to give 3-chloro-1-methylisoquinoline (2.1 g, 78%) as a solid. ¹H NMR (400 MHz, CDCl) δ ppm 2.96 (s, 3H), 7.60-7.81 (m, 2H), 8.13 (d, J=8.6 Hz, 1H), 7.76 (d, J=8.2 Hz, 1H). M.S. 177.9 (ESI) (MH⁺). A mixture of 1,1′-Bis(diphenylphosphino)ferrocene (250 mg, 0.45 mmol), zinc (88 mg, 1.35 mmol), Pd₂(dba)₃ (309 mg, 0.34 mmol), Zn(CN)₂ (793 mg, 6.76 mmol) and 3-chloro-1-methylisoquinoline (2.0 g, 11.26 mmol) in DMF (3 mL) and water (0.2 mL) was stirred at 130° C. in a microwave reactor for 15 minutes. After cooled to rt and concentrated under reduced pressure, the residue was purified by silica gel chromatography (10-50% ethyl acetate/heptane) to give 1-methylisoquinoline-3-carbonitrile (780 mg, 41.2%). M.S. 168.9 (ESI) (MH⁺). A mixture of dihydroxypalladium (209 mg, 0.15 mmol) and 1-methylisoquinoline-3-carbonitrile (500.0 mg, 2.97 mmol) in ethanol (25 mL) was placed under hydrogen at 40 psi for 5 h. The reaction mixture was filtered though diatomaceous earth and then a short pad of silica gel to give the title compound, which was used in the next step without further purification. M.S. 172.9 (ESI) (MH⁺)

### Intermediate 73

### (R)-1-(4-ethoxy-3-fluorophenyl)-N-methyl-ethanamine

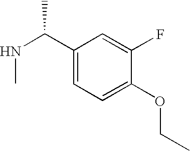

A solution of Intermediate 40 (100 mg, 0.55 mmol), di-tert-butyl dicarbonate (143 mg, 0.65 mmol) in 5 mL of methanol and 0.5 mL of 5% aq NaOH was stirred at rt for 30 minutes. The solvent was removed under reduced pressure to give crude (R)-tert-butyl 1-(4-ethoxy-3-fluorophenyl)ethylcarbamate, which was dissolved in 5 mL of THF, then sodium hydride (6.60 mg, 0.28 mmol) was added. The reaction was stirred at rt for 5 minutes, iodomethane (0.039 g, 0.28 mmol) was then added. The reaction mixture was stirred at rt for 5 h, quenched with ice water and filtered though diatomaceous earth. The filtrate was concentrated under reduced pressure. The residue was purified by silica gel column (10% heptane/ethyl acetate) to give a solid (65 mg, 79%), which was treated with TFA (2 mL) and methanol (5 mL) at rt for 16 h. The solvent was removed under reduced pressure to give the title compound, which was used in the next step without further purification. M.S. 198.2 (ESI) (MH⁺).

### Intermediate 74

### N-methyl-1-(1-methylisoquinolin-3-yl)methenamine

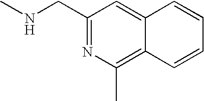

A solution of (1-methylisoquinolin-3-yl)methanamine (150 mg, 0.87 mmol) and di-tert-butyl dicarbonate (190 mg, 0.87 mmol) in 10 mL of methanol and 0.2 mL of DIPEA was stirred at 40° C. for 5 minutes. The solvent was removed under reduced pressure and the residue was dissolved in 10 mL of THF. Sodium hydride (41 mg, 1.85 mmol) was added and the reaction mixture was stirred at 0° C. for 10 minutes, iodomethane (371 mg, 2.61 mmol) was added dropwise and the reaction mixture was stirred at rt for 1 h, then ice-water was added and the reaction mixture was extracted with ether (3×) and washed with water and brine. The organic phase was dried (MgSO₄) and concentrated under reduced pressure. The residue was purified by silica gel chromatography (0-20% ethyl acetate/heptane) to give tert-butyl methyl((1-methylisoquinolin-3-yl)methyl)carbamate (205 mg, 82%) as a solid. M.S. 287.4. (ESI) (MH⁺). A solution of the above intermediate (200 mg, 0.70 mmol) in 10 mL of methanol and 1 mL of concentrated HCl was stirred at rt for 5 minutes. The solvent was removed under reduced pressure to give the title compound, which was used in the next step without further purification. M.S. 188.0 (ESI) (MH⁺).

### Intermediate 75

### 2,2,2-trifluoro-N-(isoquinolin-3-ylmethyl)ethanamine

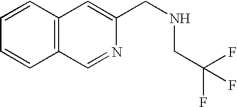

Following a procedure similar to that described in Intermediate 71 and after purification by silica gel chromatography (ethyl acetate/heptane/methanol (2/2/0.1), the title compound (58.0 mg, 12.65%) was obtained as a solid. M.S. 241.2 (ESI) (MH⁺).

### Intermediate 76

### 4-(2-dimethylamino-acetyl)-piperazine hydrochloride

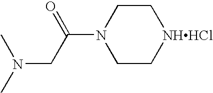

N-Boc piperazine (1.86 g, 10 mmol) and HOBT (1.35 g, 10 mmol) were added to a suspension of N,N-dimethylglycine (1.03 g, 10 mmol) in CH₂Cl₂ (20 mL), while stirring at rt under a nitrogen atmosphere. The reaction mixture was cooled in a MeOH-dry ice bath, and DCC (2.06 g, 10 mmol) was added in one portion. After 1 h, the cooling bath was removed and the reaction mixture was allowed to warm up to rt and stirred for 16 h. The reaction mixture was filtered and the filtrate was washed with 5% NaHCO₃ and water. The organic layer was concentrated under reduced pressure and the residue was purified by silica gel chromatography (4% MeOH in CH₂Cl₂) to give 4-(2-dimethylamino-acetyl)-piperazine-1-carboxylic acid tert-butyl ester (1.21 g, 44.7%) as a solid. ¹H NMR (CDCl₃) δ ppm 1.4 (s, 9H), 2.2 (s, 6H), 3.10 (s, 2H), 3.3-3.5 (m, 8H). To a solution of the above intermediate (0.54 g, 2 mmol) in CH₂Cl₂ (4 mL) was added 4M HCl in dioxane (1 mL, 4 mmol), the reaction mixture was stirred at rt for 16 h. After concentration under reduced pressure, the residue was extracted with CH₂Cl₂ and washed with a saturated aqueous solution of NaHCO₃ and then water. The organic layer was concentrated under reduced pressure to give the title compound (90 mg, 25%), which was used in the next step without further purification. ¹H NMR (CDCl₃) δ ppm 2.2 (s, 6H), 3.10 (s, 2H), 3.3-3.5 (m, 8H).

### Intermediate 77

### 2-(4-ethoxybenzyl)pyrrolidine hydrochloride

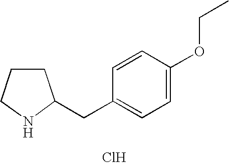

To a dry microwave tube with a magnetic stirring bar was added 1-bromo-4-ethoxybenzene (193 mg, 0.96 mmol), PdOAc₂ (3.59 mg, 2 mol %), 2,2′-oxybis(2,1-phenylene)bis(diphenylphosphine) (17 mg, 4 mol %) and Cs₂CO₃ (0.60 g, 1.84 mmol). The tube was purged with nitrogen and a solution of tert-butyl pent-4-enylcarbamate (0.148 g, 0.8 mmol, prepared according to the literature procedure as described in: Bertrand, Myra Beaudoin; Wolfe, John P. Tetrahedron 2005, 61(26), 6447-6459; Kim, Joon Young; Livinghouse, Tom. Organic Letters 2005, 7(9), 1737-1739) in anhydrous 1,4-dioxane (4 mL) was then added. The tube was sealed and heated for 50 min at 150° C. in a microwave reactor, cooled to rt and sat. NH₄Cl (2 mL) was added. The mixture was extracted with DCM (3×10 mL) and the combined organic layers were dried over Na₂SO₄, filtered and concentrated under reduced pressure. The residue was purified with silica gel chromatography (5%-30% EtOAc in Heptane) to give the title compound (0.130 g, 53.2%) as an oil. MS [M+H]⁺ 306.27 (ESI). To a solution of the above intermediate (125 mg, 0.41 mmol) in 1,4-dioxane (3 mL) was added 4N HCl (2.046 mL, 8.19 mmol) in 1,4-dioxane. The reaction mixture was stirred at rt for 5 h and concentrated under reduced pressure to give the title compounds as its hydrochloride salt, which was used for the next step without further purification. MS [M+H]⁺ 206.25 (ESI).

### Intermediate 78

### 2-(2-methoxybenzyl)pyrrolidine hydrochloride

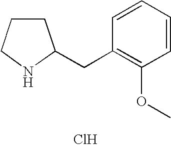

Following a procedure similar to that described for the preparation of intermediate 77 and after purification by silica gel chromatography (5%-30% EtOAc in Heptane), tert-butyl 2-(2-methoxybenzyl)pyrrolidine-1-carboxylate (0.166 g, 71.2%) was obtained as an oil. ¹H NMR (400 MHz, CDCl₃) δ ppm 1.41-1.47 (m, 9H), 1.56-1.91 (m, 4H), 2.54-2.83 (m, 1H), 2.98 (d, J=12.50 Hz, 1H), 3.21-3.43 (m, 2H), 3.74-3.82 (m, 3H), 3.88-4.19 (m, 1H), 6.76-6.94 (m, 2H), 7.02-7.22 (m, 2H). MS [M+H]⁺ 292.3 (ESI). After treatment with 4N HCl in 1,4-dioxane, the title compound was obtained as its hydrochloride salt, which was used for the next step without further purification. MS [M+H]⁺ 192.25 (ESI).

### Intermediate 79

### 2-(3-methoxybenzyl)pyrrolidine hydrochloride

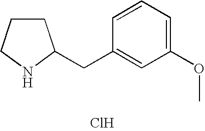

Following a procedure similar to that described for the preparation of intermediate 77 and after purification by silica gel chromatography (5%-30% EtOAc in Heptane), tert-butyl 2-(3-methoxybenzyl)pyrrolidine-1-carboxylate (0.150 g, 64.3%) was obtained as an oil. MS [M+H]⁺ 292.3 (ESI). After treatment with 4N HCl in 1,4-dioxane, the title compound was obtained as its hydrochloride salt, which was used for the next step without further purification. MS [M+H]⁺ 192.25 (ESI).

### Intermediate 80

### 2-(4-(methoxymethyl)benzyl)pyrrolidine hydrochloride

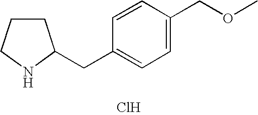

Following a procedure similar to that described for the preparation of intermediate 77 and after purification by silica gel chromatography (5%-30% EtOAc in Heptane), tert-butyl 2-(4-(methoxymethyl)benzyl)pyrrolidine-1-carboxylate (50 mg, 16.4%) was obtained as an oil. MS [M+H]⁺ 306.23 (ESI). After treatment with 4N HCl in 1,4-dioxane, the title compound was obtained as its hydrochloride salt, which was used for the next step without further purification. MS [M+H]⁺ 206.14 (ESI).

### Intermediate 81

### 1-(4-(pyrrolidin-2-ylmethyl)phenyl)ethanone hydrochloride

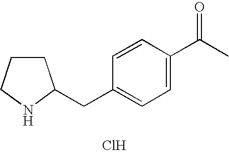

Following a procedure similar to that described for the preparation of intermediate 77, starting from 1-(4-bromophenyl)ethanol and after purification by silica gel chromatography (5%-30% EtOAc in Heptane), the ketone intermediate was obtained as an oil. MS [M+H]⁺ 304.2 (ESI). ¹H NMR (400 MHz, CDCl₃) [symbol] ppm 1.50 (s, 9H), 1.67-1.88 (m, 4H), 2.59 (s, 3H), 2.62-2.73 (m, 1H), 3.05-3.25 (m, 1H), 3.25-3.47 (m, 2H), 3.90-4.13 (m, 1H), 7.31 (d, J=8.20 Hz, 2H), 7.89 (d, J=6.25 Hz, 2H). After treatment with 4N HCl in 1,4-dioxane, the title compound was obtained as its hydrochloride salt, which was used for the next step without further purification. MS [M+H]⁺ 204.24 (ESI).

### Intermediate 82

### (R)-1-(2-(4-acetylpiperazin-1-yl)-6-isopropyl-7-oxo-6,7-dihydro-5H-pyrrolo[3,4-d]pyrimidin-4-yl)pyrrolidine-2-carboxylic acid

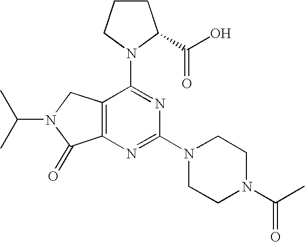

To a solution of 2,4-dichloro-6-isopropyl-5,6-dihydropyrrolo[3,4-d]pyrimidine-7-one (Intermediate 3) (0.308 g, 1.25 mmol) in BuOH (4 mL) was added (R)-pyrrolidine-2-carboxylic acid (0.151 g, 1.31 mmol) followed by DIPEA (0.162 g, 1.25 mmol). The mixture was stirred at 75° C. for 1 h, cooled to rt and the crude (R)-2-chloro-4-(2-(ethoxymethyl)pyrrolidin-1-yl)-6-isopropyl-5H-pyrrolo[3,4-d]pyrimidin-7(6H)-one solution in BuOH was transferred to a thick-walled microwave glass vial charged with a stirring bar. Then 1-(piperazin-1-yl)ethanone (0.168 g, 1.31 mmol) was added followed by DIPEA (0.218 mL, 1.25 mmol). The reaction vial was sealed and subjected to microwave radiation at 160° C. for 1 h. The mixture was concentrated under reduced pressure. The residue was taken up into DCM (30 mL) and extracted with water (2×15 mL) and brine (1×15 mL), dried over Na₂SO₄, filtered and concentrated to yield the title compound (0.429 g, 82%), which was used in the next step without further purification. MS [M+H]⁺ 417.29 (ESI).

### Intermediate 83

### 2-chloro-4-{[2-(4-chlorophenyl)-1,1-dimethylethyl]amino}-6-isopropyl-5,6-dihydro-7H-pyrrolo[3,4-d]pyrimidin-7-one

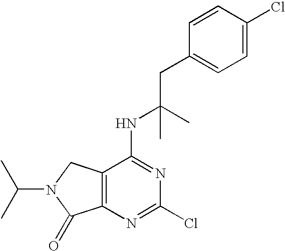

To a solution of 2,4-dichloro-6-isopropyl-5,6-dihydropyrrolo[3,4-d]pyrimidine-7-one (Intermediate 3) (150 mg, 0.610 mmol) and 1-(4-chlorophenyl)-2-methylpropan-2-amine (112 mg, 0.610 mmol) in THF (5 mL) were added diisopropylethylamine (212 μL, 1.219 mmol) and DMF (15 drops). The reaction mixture was heated in a microwave reactor at 150° C. for 2 h, cooled to rt and then concentrated under reduced pressure. The residue was purified by silica gel chromatography (20-80% ethyl acetate:hexanes) to give the title compound (112 mg, 47%) as a solid. ¹H NMR (400 MHz, CDCl₃) δ ppm 1.26 (s, 3H), 1.28 (s, 3H), 1.50 (s, 6H), 3.26 (s, 2H), 4.02 (s, 2H), 4.48 (s, 1H), 4.63-4.74 (m, 1H), 6.96 (d, J=8.20 Hz, 2H), 7.21 (d, J=8.20 Hz, 2H).

### Intermediate 84

### 2-chloro-4-(1-(4-fluorophenyl)-2-methylpropan-2-ylamino)-6-(3-methylbutan-2-yl)-5H-pyrrolo[3,4-d]pyrimidin-7(6H)-one

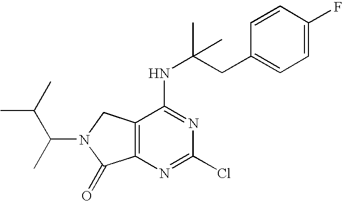

A mixture of Intermediate 20 (2,4-dichloro-6-(3-methylbutan-2-yl)-5H-pyrrolo[3,4-d]pyrimidin-7(6H)-one) (100 mg, 0.36 mmol), 1-(4-fluorophenyl)-2-methylpropan-2-amine (64.0 mg, 0.38 mmol) and DIPEA (94 mg, 0.73 mmol) in 1,2-Dichloroethane (2 mL) was stirred in a sealed tube at 175° C. for 1 h. The solvent was removed to give the title compound, which was used in the next step reaction without purification.

### Intermediate 85

### 2-chloro-4-(1-(4-fluorophenyl)-2-methylpropan-2-ylamino)-6-(1-methoxypropan-2-yl)-5H-pyrrolo[3,4-d]pyrimidin-7(6H)-one

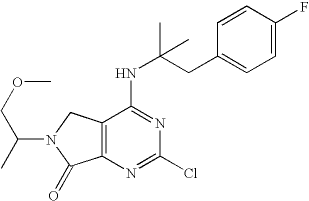

A mixture of 2,4-dichloro-6-(1-methoxypropan-2-yl)-5H-pyrrolo[3,4-d]pyrimidin-7(6H)-one (Intermediate 21) (85 mg, 0.31 mmol), 1-(4-fluorophenyl)-2-methylpropan-2-amine (54.1 mg, 0.32 mmol) and DIPEA (80 mg, 0.62 mmol) in 1,2-dichloroethane (2 mL) was stirred in a sealed tube at 175° C. for 1 h. The solvent was removed to give a residue, which was used in the next step reaction without purification.

### Intermediate 86

### 2-chloro-6-(2-chlorobenzyl)-4-(1-(4-fluorophenyl)-2-methylpropan-2-ylamino)-5H-pyrrolo[3,4-d]pyrimidin-7(6H)-one

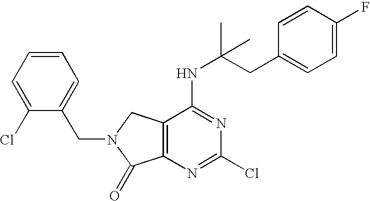

A mixture of 2,4-dichloro-6-(2-chlorobenzyl)-5H-pyrrolo[3,4-d]pyrimidin-7(6H)-one (Intermediate 22) (65 mg, 0.20 mmol), 1-(4-fluorophenyl)-2-methylpropan-2-amine (34.7 mg, 0.21 mmol) and DIPEA (51.1 mg, 0.40 mmol) in 1,2-dicholoroethane (2 mL) was stirred in a sealed tube at 175° C. for 1 h. The solvent was removed under reduced pressure to give the title compound, which was used in the next step reaction without purification.

### Intermediate 87

### 6-sec-butyl-2-chloro-4-(1-(4-fluorophenyl)-2-methylpropan-2-ylamino)-5H-pyrrolo[3,4-d]pyrimidin-7(6H)-one

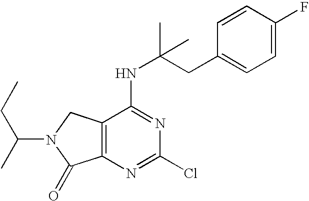

A mixture of 6-sec-butyl-2,4-dichloro-5H-pyrrolo[3,4-d]pyrimidin-7(6H)-one (Intermediate 18) (98 mg, 0.38 mmol), 1-(4-fluorophenyl)-2-methylpropan-2-amine (66.4 mg, 0.40 mmol) and DIPEA (98 mg, 0.76 mmol) in 1,2-dichloroethane (2 mL) was stirred in a sealed tube at 175° C. for 1 h. After removal of the solvent under reduced pressure, the title compound was used in the next step without purification.

### Intermediate 88

### 2-chloro-6-isopropyl-4-(1-(2-methoxyphenyl)-2-methylpropan-2-ylamino)-5H-pyrrolo[3,4-d]pyrimidin-7(6H)-one

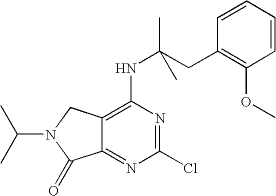

To a solution of 2,4-dichloro-6-isopropyl-5,6-dihydropyrrolo[3,4-d]pyrimidine-7-one (Intermediate 3) (452 mg, 1.84 mmol) in DCE (15 mL) was added 1-(2-methoxyphenyl)-2-methylpropan-2-amine (Intermediate 58) (329 mg, 1.84 mmol) followed by N-ethyl-N-isopropylpropan-2-amine (0.640 mL, 3.67 mmol). The reaction mixture was heated in a microwave reactor at 140° C. for 1 h. The solvent was evaporated under reduced pressure and the residue was purified by silica gel chromatography (0-10% MeOH in DCM) to give the title compound (470 mg, 65.8%). MS [M+H]⁺ 389.66 (ESI).

### Intermediate 89

### 2-chloro-6-isopropyl-4-(1-(4-methoxyphenyl)-2-methylpropan-2-ylamino)-5H-pyrrolo[3,4-d]pyrimidin-7(6H)-one

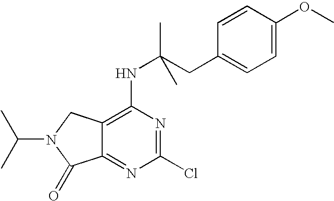

To a solution of 2,4-dichloro-6-isopropyl-5,6-dihydropyrrolo[3,4-d]pyrimidine-7-one (Intermediate 3) (247 mg, 1.00 mmol) in DCE (11 mL) was added 1-(4-methoxyphenyl)-2-methylpropan-2-amine (Intermediate 59) (180 mg, 1.00 mmol) followed by N-ethyl-N-isopropylpropan-2-amine (0.350 mL, 2.01 mmol). The reaction mixture was heated in a microwave reactor at 140° C. for 80 minutes. The solvent was evaporated under reduced pressure and the residue was purified by silica gel chromatography (0-10% MeOH in DCM) to give the title compound (390 mg, 100%). MS [M+H]⁺ 389.12 (ESI).

### Intermediate 90

### 2-chloro-6-isopropyl-4-(2-methyl-1-o-tolylpropan-2-ylamino)-5H-pyrrolo[3,4-d]pyrimidin-7(6H)-one

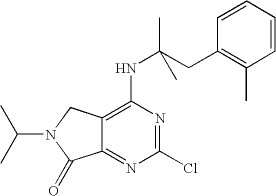

To a solution of 2,4-dichloro-6-isopropyl-5,6-dihydropyrrolo[3,4-d]pyrimidine-7-one (Intermediate 3) (271 mg, 1.10 mmol) in DCE (11 mL) was added 2-methyl-1-o-tolylpropan-2-amine (Intermediate 60) (180 mg, 1.10 mmol) followed by N-ethyl-N-isopropylpropan-2-amine (0.384 mL, 2.20 mmol). The reaction mixture was heated in a microwave reactor at 140° C. for 80 minutes. The solvent was evaporated under reduced pressure and the residue was purified by silica gel chromatography (0-10% MeOH in DCM) to give the title compound (394 mg, 96%). MS [M+H]⁺ 373.32 (ESI).

### Intermediate 91

### 2-chloro-4-(1-(4-ethoxyphenyl)-2-methylpropan-2-ylamino)-6-isopropyl-5H-pyrrolo[3,4-d]pyrimidin-7(6H)-one

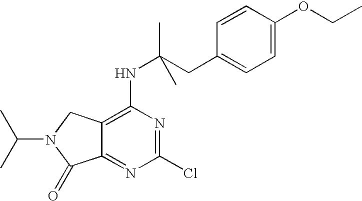

To a solution of 2,4-dichloro-6-isopropyl-5,6-dihydropyrrolo[3,4-d]pyrimidine-7-one (Intermediate 3) (185 mg, 0.75 mmol) in DCE (4 mL) was added 1-(4-ethoxyphenyl)-2-methylpropan-2-amine (Intermediate 61) (145 mg, 0.75 mmol) followed by N-ethyl-N-isopropylpropan-2-amine (0.262 mL, 1.50 mmol). The reaction mixture was heated in a microwave reactor at 140° C. for 1 h. The solvent was evaporated under reduced pressure and the residue was purified by silica gel chromatography (0-10% MeOH in DCM) to give the title compound (220 mg, 72.6%). MS [M+H]⁺ 403.23 (ESI).

### General Procedure 7 for Preparation of Intermediates 2-chlorofuro[3,4-d]pyrimidin-7(5H)-ones

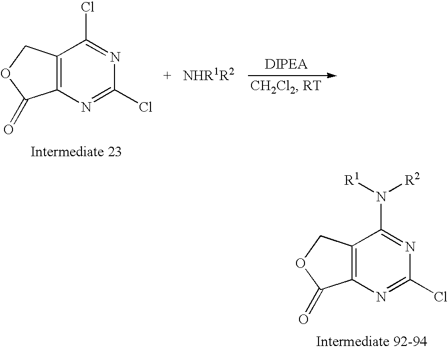

NH¹R² (10.67 mmol) followed by DIPEA (9.75 mmol) is added to a stirred solution of 2,4-dichlorofuro[3,4-d]pyrimidin-7(5H)-one (Intermediate 23) (9.75 mmol) in anhydrous CH₂Cl₂ (25 mL) at 0° C., and the reaction mixture is stirred at 0° C. for 10-15 minutes. The ice bath is removed and the reaction mixture is allowed to stir for 2 h at rt. Then 2N HCl (30 mL) is added and the solution is extracted with CH₂Cl₂ (2×100 mL). The organic extracts are washed with brine, dried over Na₂SO₄, filtered and evaporated under reduced pressure. The residue can either be triturated or recrystallized using organic solvents and the solid is filtered to give Intermediate 92-94.

### Intermediate 92

### 4-(benzhydryl-amino)-2-chloro-5H-furo[3,4-d]pyrimidin-7-one

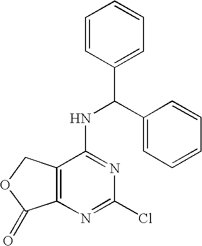

Following a procedure similar to that described in General procedure 7 and after trituration with MeOH, the title compound was obtained as a solid. (1.6 g, 47%). ¹H NMR (DMSO-d₆) δ ppm 5.31 (s, 2H), 6.52 (d, J=8.0 Hz, 1H), 7.29-7.39 (m, 10H), 9.42 (d, J=8.0 Hz, 1H).

### Intermediate 93

### 2-chloro-4-{[(4-chloro-phenyl)-phenyl-methyl]-amino}-5H-furo[3,4-d]pyrimidin-7-one

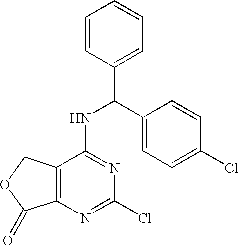

Following a procedure similar to that described in General procedure 7 and after trituration with CH₂Cl₂, the title compound was obtained as a solid (1.06 g, 57%). ¹H NMR (DMSO-d₆) δ ppm 5.32 (s, 2H), 6.51 (d, J=8.0 Hz, 1H), 7.29-7.52 (m, 9H), 9.43 (d, J=8.0 Hz, 1H).

### Intermediate 94

### 2-chloro-4-[(phenyl-p-tolyl-methyl)-amino]-5H-furo[3,4-d]pyrimidin-7-one

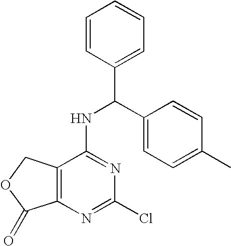

Following a procedure similar to that described in General procedure 7 and after recrystallization from MeOH:CH₂Cl₂, the title compound was obtained as a solid. (0.948 g, 53%). ¹H NMR (DMSO-d₆) δ ppm 2.28 (s, 3H), 5.30 (s, 2H), 6.44 (d, J=8.0 Hz, 1H), 7.18-7.38 (m, 9H), 9.36 (d, J=8.0 Hz, 1H).

### General Procedure 8 for Preparation of furo[3,4-d]pyrimidin-7(5H)-ones

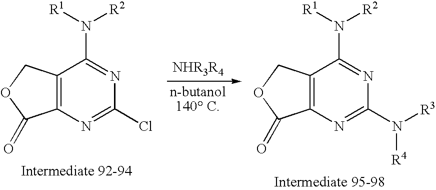

NH³R⁴ (3.44 mmol) is added to a suspension of Intermediate 92-94 (1.42 mmol) in n-butanol (3 mL) in a sealed tube, which is placed in a preheated oil bath at 140° C. for 20-25 minutes. The reaction mixture is cooled to rt then diluted with CH₂Cl₂ (10-15 mL) and water (˜20 mL). The organic layer is separated, dried over Na₂SO₄, filtered and concentrated under reduced pressure. The residue is purified by silica gel chromatography to provide Intermediate 95-98.

### Intermediate 95

### 4-(benzhydryl-amino)-2-morpholin-4-yl-5H-furo[3,4-d]-pyrimidin-7-one

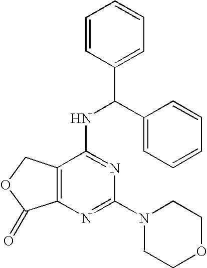

Following a procedure similar to that described in General Procedure 8, starting from 4-(benzhydryl-amino)-2-chloro-5H-furo[3,4-d]pyrimidin-7-one (Intermediate 92) and after purification by silica gel chromatography (96% EtOAc in hexanes), the title compound was obtained as a solid (0.348 g, 61%). ¹H NMR (CDCl₃) δ ppm 3.58-3.67 (m, 8H), 5.10 (s, 2H), 5.38 (br.s, 1H), 6.30 (br.s, 1H), 7.18-7.40 (m, 10H).

### Intermediate 96

### 2-(4-Acetyl-piperazin-1-yl)-4-(benzhydryl-amino)-5H-furo[3,4-d]pyrimidin-7-one

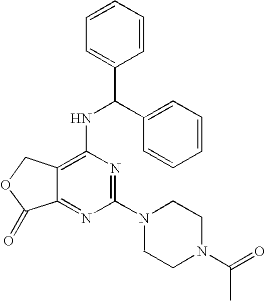

Following a procedure similar to that described in General Procedure 8, starting from 4-(benzhydryl-amino)-2-chloro-5H-furo[3,4-d]pyrimidin-7-one (Intermediate 92), the title compound was obtained as a solid (0.7 g, 62%) and used for the next step without further purification. ¹H NMR (DMSO-d₆) δ ppm 2.0 (s, 3H), 3.3-3.4 (m, 4H), 3.6-3.8 (m, 4H), 5.2 (s, 2H), 6.4 (d, J=8.0 Hz, 1H), 7.22-7.32 (m, 10H), 8.6-8.65 (m, 1H).

### Intermediate 97

### 4-{[(4-chloro-phenyl)-phenyl-methyl]amino}-2-morpholin-4-yl-5H-furo[3,4-d]pyrimidin-7-one

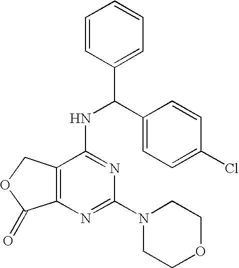

Following a procedure similar to that described in General procedure 8, starting from 2-chloro-4-{[(4-chloro-phenyl)-phenyl-methyl]-amino}-5H-furo[3,4-d]pyrimidin-7-one (intermediate 93) and after purification by silica gel chromatography (50% EtOAc in hexanes), the title compound was obtained as a solid (0.24 g, 53%). ¹H NMR (CDCl₃) δ ppm 3.60-3.67 (m, 8H), 5.11 (s, 2H), 5.19 (s, 1H), 6.24 (s, 1H), 7.22-7.38 (m, 9H).

### Intermediate 98

### 2-morpholin-4-yl-4-[(phenyl-p-tolyl-methyl-amino]-5H-furo[3,4-d]pyrimidin-7-one

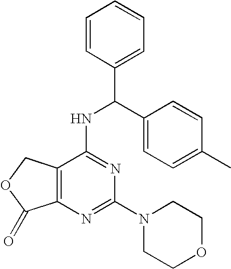

Following a procedure similar to that described in General procedure 8, starting from 2-chloro-4-[(phenyl-p-tolyl-methyl)-amino]-5H-furo[3,4-d]pyrimidin-7-one (Intermediate 94) and after purification by silica gel chromatography (50% EtOAc in hexanes), the title compound was obtained as a solid (0.616 g, 60%). ¹H NMR (CDCl₃) δ ppm 2.28 (s, 3H), 3.40-3.60 (br.s, 8H), 5.11 (s, 2H), 6.24 (s, 1H), 7.22-7.38 (m, 9H).

### General Procedure 9 for Preparation of 2-chloro-6-isopropyl-5,6-dihydro-7H-pyrrolo[3,4-d]pyrimidin-7-ones

### Intermediate 99-112

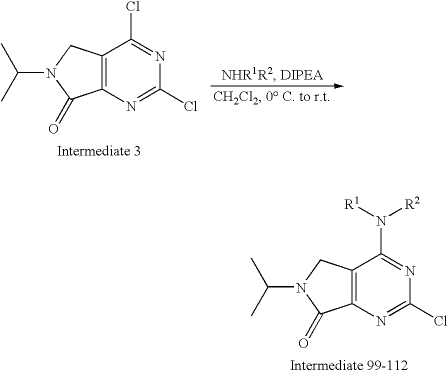

NH¹R² (1.1 mmol) followed by DIPEA (1.2 mmol) are added to a solution of 2,4-dichloro-6-isopropyl-5,6-dihydropyrrolo[3,4-d]pyrimidine-7-one (Intermediate 3) (1.0 mmol) in anhydrous CH₂Cl₂ (5 mL), cooled in an ice-water bath. The cooling bath is removed after approximately 15 minutes and the reaction mixture is allowed to warm to rt and stirred for 18 h. The reaction mixture is diluted with CH₂Cl₂ (30 mL) and washed sequentially with H₂O (10 mL), saturated aqueous NaHCO₃ (10 mL) and then with brine (10 mL). The organic layer is dried with MgSO₄, filtered then concentrated under reduced pressure. The product is purified by silica gel chromatography or trituration with organic solvents to provide the corresponding amino substituted compound.

Intermediates 99-112 were synthesized using a procedure similar to that described in General procedure 9 and substituting for the appropriate starting materials.

| | | | |
|---|---|---|---|
| Intermediate | | | |
| # | Structure | Name | Analytical Data |

| | | | |
|---|---|---|---|
| 99 | 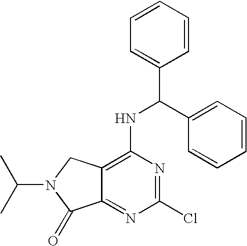 | 2-chloro-6-isopropyl-4-{[(4-methoxy-phenyl)-phenyl-methyl]-amino}-5,6-dihydro-pyrrolo[3,4-d]pyrimidin-7-one | ¹H NMR (DMSO-d₆) δ ppm1.20-1.25 (m, 6 H), 4.4-4.45 (m,3 H), 6.50-6.55 (m, 1 H), 7.20-7.7.35 (m, 10 H), 9.0-9.05 (m,1 H). |
| 100 | 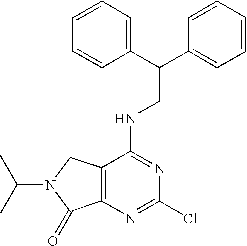 | 2-chloro-4-(2,2-diphenyl-ethylamino)-6-isopropyl-5,6-dihydro-pyrrolo[3,4-d]pyrimidin-7-one | ¹H NMR (DMSO-d₆) δ ppm1.18 (d, J = 7 Hz, 6 H), 4.04 (t, J = 6.4Hz, 2 H), 4.09 (s, 2 H), 4.36-4.30(m, 1 H), 4.41 (m, 1 H), 7.22-7.18(m, 2 H), 7.35-7.30 (m, 8 H), 8.41(t, 1 H). |
| 101 | 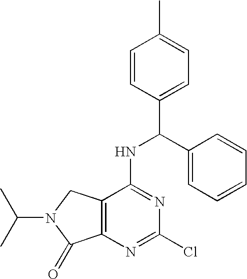 | 2-chloro-6-isopropyl-4-[(phenyl-p-tolyl-methyl)-amino]-5,6-dihydro-pyrrolo[3,4-d]pyrimidin-7-one | ¹H NMR (CDCl₃) δ ppm 1.23(d, J = 6.0 Hz, 6 H), 2.35 (s, 3 H),4.15 (s, 2 H), 4.57-4.64 (m, 1 H),5.96 (d, J = 7.6 Hz, 1 H), 7.17-7.35 (m, 9 H). |
| 102 | 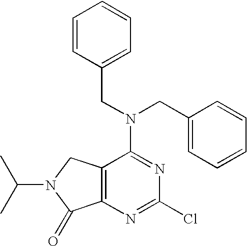 | 2-chloro-4-dibenzylamino-6-isopropyl-5,6-dihydro-pyrrolo[3,4-d]pyrimidin-7-one | ¹H NMR (CDCl₃) δ ppm 1.12(d, J = 3.0 Hz, 6 H), 4.19 (s, 2 H),4.58-4.61 (m, 1 H), 4.82 (s, 4 H),7.22-7.39 (m, 10 H). |
| 103 | 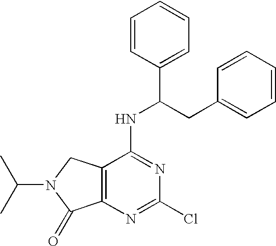 | 2-chloro-4-(1,2-diphenyl-ethylamino)-6-isopropyl-5,6-dihydro-pyrrolo[3,4-d]pyrimidin-7-one | ¹H NMR (CDCl₃) δ ppm 1.30-1.28 (m, 6 H), 3.17-3.31 (m, 2 H),3.95-4.19 (m, 2 H), 4.61 (m, 1 H),5.50 (br. s, 1 H), 7.10-7.40 (m,10 H). |
| 104 | 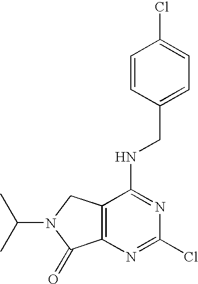 | 2-chloro-4-(4-chloro-benzyl)-6-isopropyl-5,6-dihydro-pyrrolo[3,4-d]primidin-7-one | ¹H NMR (CD₃OD) δ ppm 1.30(d, J = 8.0 Hz, 6 H), 4.29 (s, 2 H),4.51 (m, 1 H), 4.70 (s, 2 H), 7.31-7.38 (m, 4 H). |
| 105 | 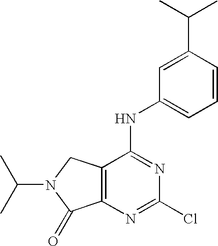 | 2-chloro-6-isopropyl-4-(3-isopropyl-phenylamino)-5,6-dihydro-pyrrolo[3,4-d]pyrimidin-7-one | ¹H NMR (CDCl₃) δ ppm 1.14(d, 6 H), 1.25 (d, 6 H), 2.96 (m, 1 H),3.69 (s, 2 H), 4.58-4.65 (m, 1 H),6.99 (s, 1 H), 7.08-7.52 (m, 4 H). |
| 106 | 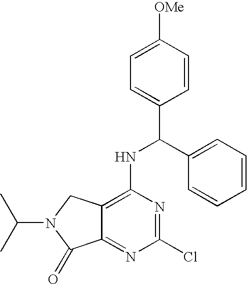 | 2-chloro-6-isopropyl-4-{[(4-methoxyphenyl)(phenyl)methyl]amino}-5,6-dihydro-7H-pyrrolo[3,4-d]pyrimidin-7-one | ¹H NMR (CDCl₃) δ ppm 1.20-1.25 (m, 6 H), 3.8 (s, 3 H), 4.2(br. s, 2 H), 4.55-4.59 (m, 1 H),5.85 (s, 1 H), 6.85 (m, 2 H), 7.19(m, 2 H), 7.3-7.44 (m, 5 H). |
| 107 | 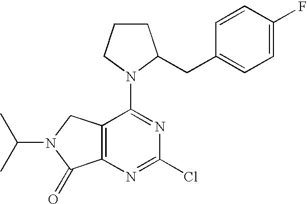 | 2-chloro-4-[2-(4-fluorobenzyl)-pyrrolidin-1-yl]-6-isopropyl-5H-pyrrolo[3,4-d]pyrimidin-7(6H)-one | ¹H NMR (CDCl₃) δ ppm 1.28(d, 6 H), 1.74-1.90 (m, 2 H), 1.92-2.12 (m, 2 H), 2.44-2.58 (m, 1 H),3.17-3.30 (m, 1 H), 3.60-3.84 (m, 2 H), 4.38-4.60 (m,3 H), 4.64-4.80 (m, 1 H), 6.94-7.08 (m, 2 H), 7.18-7.30 (m, 2 H). |
| 108 | 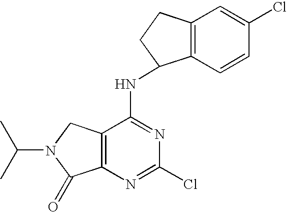 | 2-chloro-4-(5-chloro-2,3-dihydro-1H-inden-1-ylamino)-6-isopropyl-5H-pyrrolo[3,4-d]primidin-7(6H)-one | ¹H NMR (400 MHz, CDCl₃) δppm 1.23 (d, J = 6.80 Hz, 6 H),2.04-2.13 (m, 1 H), 2.67-2.75(m, 1 H), 2.85-2.91 (m, 1 H),2.99-3.07 (m, 1 H), 4.22-4.41(m, 2 H), 4.45 (dt, J = 13.60,6.80, 1 H), 5.80 (br s, 1 H), 6.51(br s, 1 H), 7.03 (d, J = 8.0 Hz,1 H), 7.15-7.17 (m, 2 H). M.S. (calcd): 378.28 (MH⁺), M.S.(found): 378.17 (ESI) (MH⁺). |
| 109 | 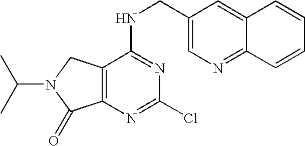 | 2-chloro-6-isopropyl-4-(quinolin-3-ylmethylamino)-5H-pyrrolo[3,4-d]pyrimidin-7(6H)-one | ¹H NMR (400 MHz, DMSO-d₆)δ ppm 1.20 (d, J = 6.63 Hz, 6 H),4.28 (s, 2 H), 4.36 (dt, J = 13.27,6.63 Hz, 1 H), 4.82 (d, J = 3.90Hz, 2 H), 7.60 (t, J = 7.42 Hz, 1 H),7.73 (t, J = 7.03 Hz, 1 H), 7.95(d, J = 8.20 Hz, 1 H), 8.01(d, J = 8.20 Hz, 1 H), 8.28 (s,1 H), 8.88 (br. s., 1 H), 8.93 (d, J = 1.95 Hz, 1 H). M.S. (calcd):368.84 (MH⁺), M.S, (found):368.55 (ESI) (MH⁺). |
| 110 | 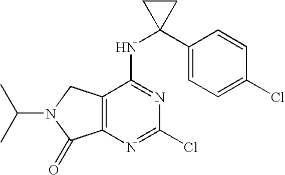 | 2-chloro-4-[1-(4-fluorophenyl)-cyclopropylamino]-6-isopropyl-5H-pyrrolo[3,4-d]pyrimidin-7(6H)-one | ¹H NMR (DMSO-d₆) δ ppm 1.22(d, 6 H), 1.25-1.40 (m, 4 H),4.29 (s, 2 H), 4.33-4.45 (m, 1 H),7.16-7.38 (m, 4 H), 8.97 (s, 1 H). |
| 111 | 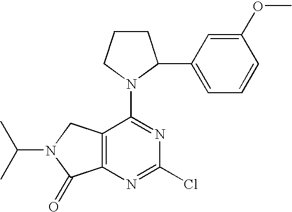 | 2-chloro-6-isopropyl-4-(2-(3-methoxyphenyl)pyrrolidin-1-yl)-5H-pyrrolo[3,4-d]pyrimidin-7(6H)-one | ¹H NMR (CDCl₃) δ ppm 0.82-1.27 (m, 6 H), 1.92-2.13 (m,3 H), 2.38-2.52 (m, 1 H), 3.74-3.90 (m, 1 H), 3.79 (s, 3 H), 4.04-4.12 (m, 1 H), 4.19-4.42 (m,1 H), 4.54-4.64 (m, 1 H), 5.04-5.30 (m, 1 H), 6.65 (br s, 1 H),6.71 (d, J = 7.41 Hz, 1 H), 6.71(d, J = 7.41 Hz, 1 H), 6.81 (dd, J =7.80 Hz, J = 1.95 Hz, 1 H),7.28(dd, J = 7.80 Hz, J = 7.80Hz, 1 H). |
| 112 | 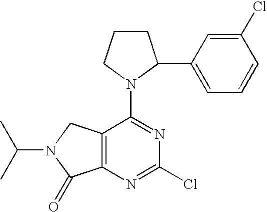 | 2-chloro-4-(2-(3-chlorophenyl)pyrrolidin-1-yl)-6-isopropyl-5H-pyrrolo[3,4-d]pyrimidin-7(6H)-one | ¹H NMR (DMSO-d₆) δppm 1.22-1.31 (m, 6 H), 1.80-2.05 (m,4 H), 2.24-2.52 (m, 2 H), 3.89-4.04 (m, 1 H), 4.16-4.29 (m,1 H), 4.36-4.48 (m, 1 H), 5.33-5.42 (m, 1 H), 7.16-7.43 (m,4 H). |

### Intermediate 113

### 2-chloro-6-isopropyl-4-(isoquinolin-3-ylmethylamino)-5H-pyrrolo[3,4-d]pyrimidin-7(6H)-one

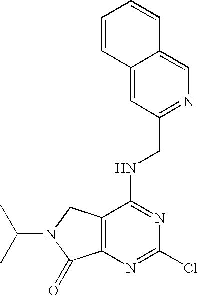

2,4-Dichloro-6-isopropyl-5,6-dihydropyrrolo[3,4-d]pyrimidine-7-one (Intermediate 3) (500 mg, 2.03 mmol), isoquinolin-3-ylmethanamine (418 mg, 2.64 mmol) and DIPEA (0.708 mL, 4.06 mmol) were combined in n-BuOH (17 mL) and heated in a microwave reactor at 65° C. for 30 minutes. After concentration under reduced pressure, the crude was purified by silica gel chromatography (MeOH/DCM 1-10%) to give the title compound (747 mg, 100%) as foam. MS [M+H]⁺ 368.05 (ESI).

### Intermediate 114

### 2-chloro-4-[2-(4-fluoro-phenyl)-1,1-dimethyl-ethylamino]-6-isopropyl-5,6-dihydro-pyrrolo[3,4-d]pyrimidin-7-one

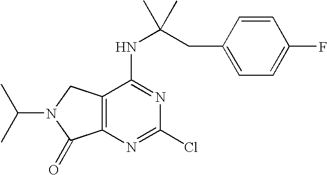

2,4-Dichloro-6-isopropyl-5,6-dihydropyrrolo[3,4-d]pyrimidine-7-one (Intermediate 3) (1.23 g, 5 mmol) was added to a solution of 2-(4-fluoro-phenyl)-1,1-dimethyl-ethylamine (0.92 g, 5.5 mmol) and DIPEA (1.74 mL, 10 mmol) in 1,2-dichloroethane (22 mL). The solution was sealed in a glass pressure vessel and heated at 140° C. for 18 h. The reaction mixture was cooled to rt, concentrated under reduced pressure and the residue was dissolved in CH₂Cl₂ and washed with water. The organic layer was dried with MgSO₄, filtered and concentrated under reduced pressure. The residue was purified by silica gel chromatography (EtOAc: CH₂Cl₂) to provide the title compound as a solid (0.65 g, 35%). ¹H NMR (400 MHz, CDCl₃) δ ppm 1.20 (s, 3H), 1.25 (s, 3H), 1.48 (s, 6H), 3.22 (s, 2H), 4.05 (s, 2H), 3.95 (s, 1H), 4.62-4.67 (m, 1H), 6.90-7.01 (m, 4H).

### Intermediate 115

### 2-chloro-6-isopropyl-4-(2-methyl-1-p-tolylpropan-2-ylamino)-5H-pyrrolo[3,4-d]pyrimidin-7(6H)-one

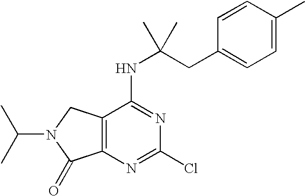

To a solution of 2,4-dichloro-6-isopropyl-5,6-dihydropyrrolo[3,4-d]pyrimidine-7-one (Intermediate 3) (100 mg, 0.41 mmol) in DCM (2 mL) was added 2-methyl-1-p-tolylpropan-2-amine hydrochloride (Intermediate 28) (81 mg, 0.41 mmol) followed by DIPEA (0.142 mL, 0.82 mmol) at rt. The reaction was heated in a microwave reactor at 140° C. for 2 h. After concentration under reduced pressure, the residue was purified by preparative LCMS (high pH) to give the title compound (16 mg, 11%). M.S. (found): 373.3 (ESI) (MH⁺).

### Intermediate 116

### 2-chloro-4-((cyclopropylmethyl)(4-ethoxybenzyl)amino)-6-isopropyl-5H-pyrrolo[3,4-d]pyrimidin-7(6H)-one

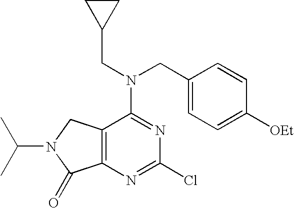

To a solution of 2,4-dichloro-6-isopropyl-5H-pyrrolo[3,4-d]pyrimidin-7(6H)-one (Intermediate 3) (100 mg, 0.41 mmol) in DCM (2 mL) was added 1-cyclopropyl-N-(4-ethoxybenzyl)methenamine (Intermediate 27) (83 mg, 0.41 mmol) followed by DIPEA (0.071 mL, 0.41 mmol). The reaction was heated in a microwave reactor for 30 minutes at 70° C. After concentration under reduced pressure, the residue was purified by silica gel chromatography (30-60% EtOAc/Heptane) to afford the title compound (140 mg, 83%). M.S. (found): 415.0 (ESI) (MH⁺).

### Intermediate 117

### tert-butyl 4-{4-[(diphenylmethyl)amino]-6-isopropyl-7-oxo-6,7-dihydro-5H-pyrrolo[3,4-d]pyrimidin-2-yl}piperazine-1-carboxylate

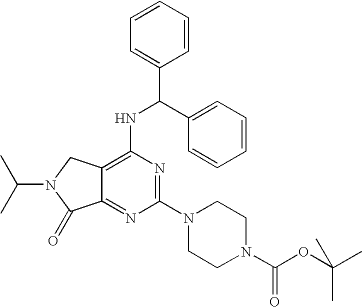

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (0.12 g, 73%). ¹H NMR (CDCl₃) δ ppm 1.20-1.25 (m, 6H), 1.4 (s, 9H), 3.25-3.35 (m, 4H), 3.60-3.70 (m, 4H), 4.05-4.10 (br.s, 2H), 4.60-4.65 (m, 1H), 5.05-5.10 (m, 1H), 6.30-6.35 (m, 1H), 7.20-7.30 (m, 10H).

### Intermediate 118

### 4-[2-(4-Fluoro-phenyl)-1,1-dimethyl-ethylamino]-2-(3-hydroxymethyl-piperazin-1-yl)-6-isopropyl-5,6-dihydro-pyrrolo[3,4-d]pyrimidin-7-one

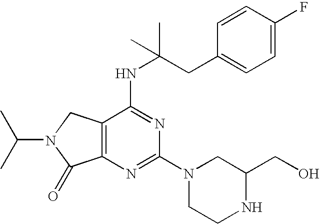

Following a procedure similar to that described in General Procedure 4, starting from 2-chloro-4-[2-(4-fluoro-phenyl)-1,1-dimethyl-ethylamino]-6-isopropyl-5,6-dihydro-pyrrolo[3,4-d]pyrimidin-7-one (Intermediate 114) and after purification by preparative HPLC (Waters XTerra Prep C₁₈, 5 μm, 30×100 mm), the title compound was obtained as an oil (80 mg, 40%). ¹H NMR (400 MHz, CDCl₃) δ ppm 1.24 (d, J=6.73 Hz, 6H), 1.45 (s, 6H), 2.87-2.94 (m, 1H), 2.99 (td, J=6.81, 2.49 Hz, 1H), 3.11-3.18 (m, 1H), 3.20-3.29 (m, 4H), 3.62 (dd, J=10.83, 6.73 Hz, 1H), 3.76 (dd, J=10.98, 4.24 Hz, 1H), 3.91 (s, 2H), 4.01 (s, 1H), 4.56-4.67 (m, 3H), 6.89-7.01 (m, 4H).

### Intermediate 119

### tert-butyl[2-({4-[(2,2-diphenylethyl)amino]-6-isopropyl-7-oxo-6,7-dihydro-5H-pyrrolo[3,4-d]pyrimidin-2-yl}amino)ethyl]methylcarbamate

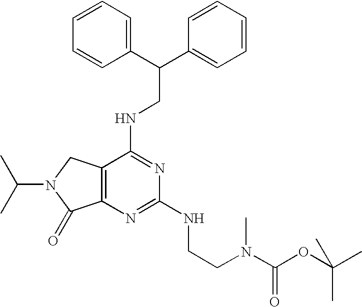

Following a procedure similar to that described in General Procedure 4, starting from 2-chloro-4-(2,2-diphenyl-ethylamino)-6-isopropyl-5,6-dihydro-pyrrolo[3,4-d]pyrimidin-7-one (Intermediate 100) and after recrystallization from EtOAc and hexanes, the title compound was obtained as a solid (0.114 g, 57%). ¹H NMR (CDCl₃) δ ppm 1.20 (d, 6H), 1.42 (s, 9H), 2.83 (s, 3H), 3.44 (br.s, 2H), 3.61 (br.s, 2H), 3.84 (s, 2H), 4.13 (br.s, 2H), 4.34 (br.s, 2H), 4.68-4.61 (m, 1H), 7.31-7.26 (m, 6H), 7.35-7.32 (m, 4H).

### Intermediate 120

### (S)-tert-butyl 4-(6-isopropyl-4-(isoquinolin-3-ylmethylamino)-7-oxo-6,7-dihydro-5H-pyrrolo[3,4-d]pyrimidin-2-yl)-2-methylpiperazine-1-carboxylate

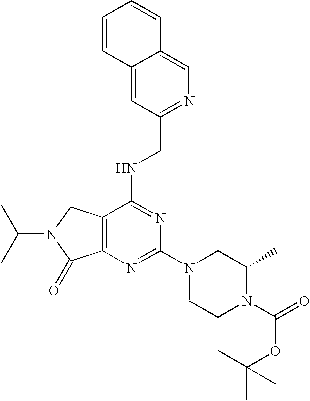

2-Chloro-6-isopropyl-4-(isoquinolin-3-ylmethylamino)-5H-pyrrolo[3,4-d]pyrimidin-7(6H)-one (Intermediate 113) (230 mg, 0.63 mmol), (S)-tert-butyl 2-methylpiperazine-1-carboxylate (138 mg, 0.69 mmol) and DIPEA (0.218 mL, 1.25 mmol) were combined in n-BuOH (6 mL) and heated in a microwave reactor at 160° C. for 30 minutes. After concentration under reduced pressure, the crude was purified by preparative HPLC (gradient 35-55% CH₃CN in H₂O containing 10 mM NH₄HCO₃) to give the title compound (51.0 mg, 15.3%) as a solid. ¹H NMR (400 MHz, CDCl₃) δ ppm 1.06 (d, J=6.64 Hz, 3H), 1.26 (d, J=6.64 Hz, 6H), 1.47 (s, 9H), 2.99 (td, J=12.01, 2.93 Hz, 1H), 3.13 (qd, 2H), 3.86 (d, J=12.11 Hz, 1H), 4.14 (s, 2H), 4.28 (s, 1H), 4.60 (d, J=13.28 Hz, 1H), 4.63-4.72 (m, 1H), 4.72-4.77 (m, 1H), 4.86-4.99 (m, 2H), 5.78 (t, J=5.27 Hz, 1H), 7.57-7.65 (m, 1H), 7.68 (s, 1H), 7.69-7.74 (m, 1H), 7.80 (d, 1H), 7.98 (d, J=8.20 Hz, 1H), 9.24 (s, 1H). MS [M+H]⁺ 532.3 (ESI).

### Intermediate 121

### (R)-tert-butyl 4-(6-isopropyl-4-(isoquinolin-3-ylmethylamino)-7-oxo-6,7-dihydro-5H-pyrrolo[3,4-d]pyrimidin-2-yl)-2-methylpiperazine-1-carboxylate

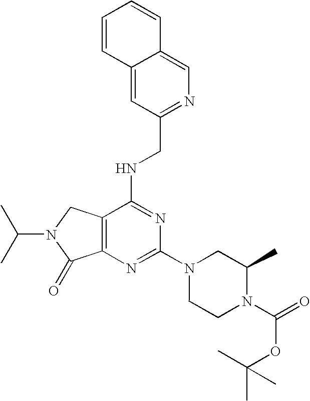

2-Chloro-6-isopropyl-4-(isoquinolin-3-ylmethylamino)-5H-pyrrolo[3,4-d]pyrimidin-7(6H)-one (Intermediate 113) (230 mg, 0.63 mmol), (R)-tert-butyl 2-methylpiperazine-1-carboxylate (138 mg, 0.69 mmol) and DIPEA (0.218 mL, 1.25 mmol) were combined in n-BuOH (6 mL) and heated in a microwave reactor at 170° C. for 30 minutes. After concentration under reduced pressure, the crude was purified by preparative HPLC (gradient 35-55% CH₃CN in H₂O containing 10 mM NH₄HCO₃) to give the title compound (49.0 mg, 14.7%). ¹H NMR (400 MHz, CDCl₃) δ ppm 1.06 (d, J=6.64 Hz, 3H), 1.26 (d, J=6.64 Hz, 6H), 1.47 (s, 9H), 2.99 (td, J=12.40, 3.71 Hz, 1H), 3.14 (qd, 2H), 3.87 (d, J=13.28 Hz, 1H), 4.15 (s, 2H), 4.29 (s, 1H), 4.60 (d, J=13.67 Hz, 1H), 4.63-4.72 (m, 1H), 4.75 (d, J=11.72 Hz, 1H), 4.87-4.99 (m, 2H), 5.74 (t, J=5.08 Hz, 1H), 7.58-7.64 (m, 1H), 7.68 (s, 1H), 7.69-7.75 (m, 1H), 7.78-7.82 (m, 1H), 7.99 (d, J=8.20 Hz, 1H), 9.24 (s, 1H). MS [M+H]⁺ 532.3 (ESI).

### Intermediate 122

### (S)-tert-butyl 4-(6-isopropyl-7-oxo-4-(quinolin-3-ylmethylamino)-6,7-dihydro-5H-pyrrolo[3,4-d]pyrimidin-2-yl)-2-methylpiperazine-1-carboxylate

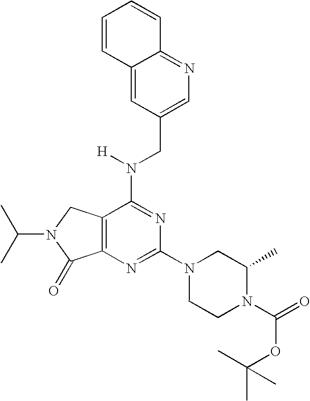

2-Chloro-6-isopropyl-4-(quinolin-3-ylmethylamino)-5H-pyrrolo[3,4-d]pyrimidin-7(6H)-one (Intermediate 109) (230 mg, 0.63 mmol), (S)-tert-butyl 2-methylpiperazine-1-carboxylate (138 mg, 0.69 mmol) and DIPEA (0.218 mL, 1.25 mmol) were combined in n-BuOH (6 mL) and heated in a microwave reactor at 160° C. for 40 minutes. After concentration under reduced pressure, the crude was purified by preparative HPLC (gradient 45-65% CH₃CN in H₂O containing 10 mM NH₄HCO₃) to give the title compound (179 mg, 53.8%). ¹H NMR (400 MHz, CDCl₃) δ ppm 1.03 (d, J=5.86 Hz, 3H), 1.24 (d, J=6.64 Hz, 6H), 1.46 (s, 9H), 2.99 (td, J=12.30, 3.12 Hz, 1H), 3.10 (td, J=12.70, 3.13 Hz, 1H), 3.17 (d, J=11.72 Hz, 1H), 3.85 (d, J=12.50 Hz, 1H), 4.09 (s, 2H), 4.25 (s, 1H), 4.53 (d, J=13.28 Hz, 1H), 4.60-4.78 (m, 2H), 4.83-4.97 (m, 2H), 5.07 (t, J=5.86 Hz, 1H), 7.53-7.59 (m, 1H), 7.69-7.75 (m, 1H), 7.77 (dd, J=8.20, 1.17 Hz, 1H), 8.07-8.13 (m, 2H), 8.92 (d, J=1.95 Hz, 1H). MS [M+H]⁺ 532.3 (ESI).

### Intermediate 123

### (R)-tert-butyl 4-(6-isopropyl-7-oxo-4-(quinolin-3-ylmethylamino)-6,7-dihydro-5H-pyrrolo[3,4-d]pyrimidin-2-yl)-2-methylpiperazine-1-carboxylate

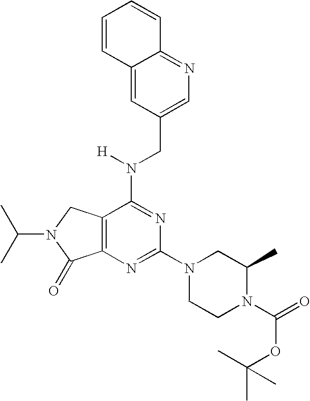

2-Chloro-6-isopropyl-4-(quinolin-3-ylmethylamino)-5H-pyrrolo[3,4-d]pyrimidin-7(6H)-one (Intermediate 109) (230 mg, 0.63 mmol), (R)-tert-butyl 2-methylpiperazine-1-carboxylate (163 mg, 0.81 mmol) and DIPEA (0.218 mL, 1.25 mmol) were combined in n-BuOH (6 mL) and heated in a microwave reactor at 160° C. for 30 minutes. After concentration under reduced pressure and the crude was purified by preparative HPLC (gradient 45-65% CH₃CN in H₂O containing 10 mM NH₄HCO₃) to give the title compound (206 mg, 62.0%). ¹H NMR (400 MHz, CDCl₃) δ ppm 1.01 (d, J=6.64 Hz, 3H), 1.21 (d, J=6.64 Hz, 6H), 1.45 (s, 9H), 2.96 (td, J=12.11, 2.73 Hz, 1H), 3.08 (td, J=12.70, 3.52 Hz, 1H), 3.14 (dd, J=13.67, 3.52 Hz, 1H), 3.82 (d, J=13.28 Hz, 1H), 4.09 (s, 2H), 4.23 (s, 1H), 4.50 (d, J=13.28 Hz, 1H), 4.57-4.71 (m, 2H), 4.81-4.95 (m, 2H), 5.50 (t, J=5.47 Hz, 1H), 7.50-7.58 (m, 1H), 7.67-7.73 (m, 1H), 7.74 (dd, J=8.20, 1.17 Hz, 1H), 8.05-8.10 (m, 2H), 8.90 (d, J=2.34 Hz, 1H). MS [M+H]⁺ 532.3 (ESI).

### Intermediate 124

### 2-(4-acetylpiperazin-1-yl)-4-[(2R)-2-(4-hydroxy-3-fluorophenyl)pyrrolidin-1-yl]-6-(1-methylethyl)-5,6-dihydro-7H-pyrrolo[3,4-d]pyrimidin-7-one

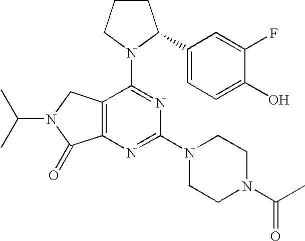

Following a procedure similar to that described in General Procedure 6 and after concentration under reduced pressure, the title compound (1.5 mmol scale, HPLC purity>85%) was used in the next step without further purification. MS [M+H]⁺ 483.33 (ESI).

### Intermediate 125

### 6-isopropyl-2-(piperazin-1-yl)-4-(quinolin-3-ylmethylamino)-5H-pyrrolo[3,4-d]pyrimidin-7(6H)-one

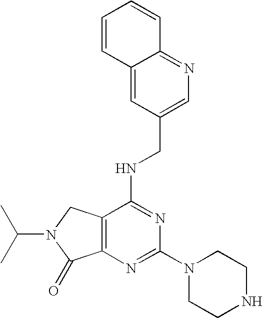

2-Chloro-6-isopropyl-4-(quinolin-3-ylmethylamino)-5H-pyrrolo[3,4-d]pyrimidin-7(6H)-one (Intermediate 109) (147 mg, 0.4 mmol), tert-butyl piperazine-1-carboxylate (78 mg, 0.42 mmol) and DIPEA (54 mg, 0.42 mmol) were combined in i-PrOH (2 mL) and heated in a microwave reactor at 160° C. for 1 h. After concentration under reduced pressure, the crude tert-butyl 4-(6-isopropyl-7-oxo-4-(quinolin-3-ylmethylamino)-6,7-dihydro-5H-pyrrolo[3,4-d]pyrimidin-2-yl)piperazine-1-carboxylate (0.198 g, 96%) was dissolved in DCM (4 mL) and added TFA (0.295 mL, 3.83 mmol). The reaction mixture was stirred at 25° C. for 5 h. After concentration under reduced pressure, the title compound was obtained as its TFA salt, which was used in the next step without further purification. MS [M+H]⁺ 418.30 (ESI).

### Intermediate 126

### (R)-1-(6-ethoxy-5-fluoropyridin-3-yl)ethanamine

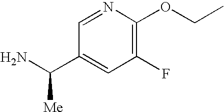

Following a procedure similar to that described for Intermediate 40, the title compound (246 mg) was obtained and was used without further purification. ¹H NMR (400 MHz, DMSO-d₆) δ ppm 1.33 (t, J=7.03 Hz, 3H), 1.53 (d, J=6.64 Hz, 3H), 4.23-4.56 (m, 3H), 8.01 (dd, J=11.72, 1.95 Hz, 1H), 8.11 (s, 1H), 8.76 (br. s., 2H).

### Intermediate 127

### (R)-1-(6-ethoxy-5-methylpyridin-3-yl)ethanamine

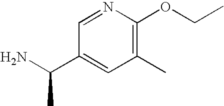

Following a procedure similar to that described for Intermediate 40, the title compound (64 mg, 89% over 3 steps) was obtained and was used without further purification. ¹H NMR (400 MHz, CD₃OD) δ ppm. 1.44 (t, J=7.03 Hz, 3H), 1.64 (d, J=6.64 Hz, 3H), 2.27 (s, 3H), 4.36-4.57 (m, 3H), 7.79 (s, 1H), 8.08 (d, J=2.34 Hz, 1H).

### Intermediate 128

### 4-((tert-butyldimethylsilyloxy)methyl)benzaldehyde

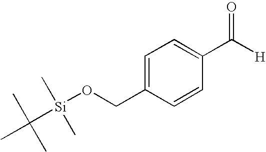

To a solution of tert-butyldimethylchlorosilane (2.66 g, 17.63 mmol) and imidazole (2.50 g, 36.72 mmol) in DMF (25 mL) was added a solution of 4-(hydroxymethyl)benzaldehyde (2 g, 14.69 mmol) in DMF (25.00 mL) at 0° C. The reaction mixture was stirred at rt for 2 h, then taken up into EtOAc (250 mL) and washed with water (5×) and brine. The organic layer was separated and dried (MgSO₄), filtered and concentrated under reduced pressure to give the title compound (3.96 g, quantitative yield), which was used in the next step without purification. ¹H NMR (400 MHz, CDCl₃) δ ppm. 0.13 (s, 6H), 0.96 (s, 9H), 4.83 (s, 2H), 7.50 (d, J=7.81 Hz, 2H), 7.86 (d, J=8.20 Hz, 2H), 10.01 (s, 1H).

### Intermediate 129

### (R)-(4-(1-aminoethyl)phenyl)methanol

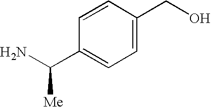

Following a procedure similar to that described for Intermediate 40, starting from 4-((tert-butyldimethylsilyloxy)methyl)benzaldehyde (Intermediate 128), the title compound (395 mg, 64% over 3 steps) was obtained and was used without further purification. [α]_{D}=+4.2 (c=0.01, MeOH) ¹H NMR (400 MHz, CD₃OD) δ ppm 1.16-1.21 (m, 2H), 1.57-1.69 (m, 3H), 4.45 (q, J=6.90 Hz, 1H), 4.63 (s, 2H), 7.43 (s, 4H).

### Intermediate 130

### 2-methyl-1-m-tolylpropan-2-amine

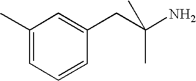

Following a procedure similar to that described for Intermediate 58, the title compound was obtained (0.677 g, 50.1% over 3 steps), which was used in the next step without further purification. MS [M+H]⁺ 164.20 (ESI).

### Intermediate 131

### 2-chloro-6-isopropyl-4-(2-methyl-1-m-tolylpropan-2-ylamino)-5H-pyrrolo[3,4-d]pyrimidin-7(6H)-one

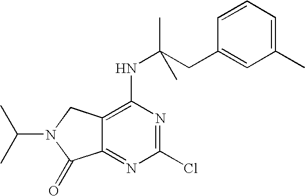

Following a procedure similar to that described for the preparation of intermediate 88, starting from 2-methyl-1-m-tolylpropan-2-amine (Intermediate 130) and after purification by silica gel chromatography (0-10% MeOH in DCM), the title compound (630 mg, 92%) was obtained as a solid. MS [M+H]⁺ 373.01 (ESI).

### Intermediate 132

### 4-ethoxy-2-methoxybenzaldehyde

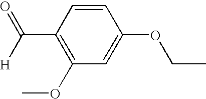

Following a procedure similar to that described for the preparation of Intermediate 29, to a solution of 4-hydroxy-2-methoxybenzaldehyde (1 g, 6.57 mmol) in DMF (15 mL) was added potassium carbonate (1.363 g, 9.86 mmol) followed by iodoethane (0.796 mL, 9.86 mmol) at rt. The reaction mixture was stirred at 50° C. for 18 h. Water was added and extracted with EtOAc (2×). The combined organic phases were dried (MgSO₄), filtered and concentrated under reduced pressure to give the title compound, which was used without further purification. ¹H NMR (400 MHz, CDCl₃) δ ppm 1.45 (t, J=7.03 Hz, 3H), 3.90 (s, 3H), 4.11 (q, J=7.03 Hz, 2H), 6.44 (d, J=1.95 Hz, 1H), 6.53 (dd, J=8.79, 2.15 Hz, 1H), 7.80 (d, J=8.59 Hz, 1H), 10.28 (s, 1H).

### Intermediate 133

### 1-(4-ethoxy-2-methoxyphenyl)-N-methylmethanamine

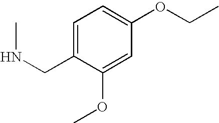

To a solution of 4-ethoxy-2-methoxybenzaldehyde (Intermediate 132, 0.3 g, 1.66 mmol) was added methanamine (0.717 mL, 8.32 mmol) followed by sodium triacetoxyhydroborate (0.353 g, 1.66 mmol) and acetic acid (10.00 mg, 0.17 mmol) at rt. The reaction mixture was stirred at rt for 16 h. Water was added and the mixture was extracted with DCM. The aqueous layer was evaporated under reduced pressure and DMF (20 mL) was added, filtered and concentrated under reduced pressure to give title compound, which was used without further purification. MS [M+H]⁺ 195.97 (ESI).

### Intermediate 134

### (4-ethoxy-2-methoxyphenyl)methanamine

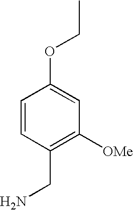

A solution of 4-ethoxy-2-methoxybenzaldehyde (Intermediate 132, 0.3 g, 1.66 mmol), sodium acetate (0.137 g, 1.66 mmol) and hydroxylamine hydrochloride (0.174 g, 2.50 mmol) in ethanol (10 mL) and water (1.5 mL) was stirred at reflux for 18 h. After cooling to rt, the solvents were removed under reduced pressure and the residue was added saturated aqueous solution of NaHCO₃ (15 mL), extracted with ethyl acetate (3×20 mL). The organic layers were combined, dried over MgSO₄, filtered and concentrated under reduced pressure to give 4-ethoxy-2-methoxybenzaldehyde oxime (0.285 g, 88%), which was used in next step without further purification. M.S. [M+H]⁺ 195.94 (ESI). The crude 4-ethoxy-2-methoxybenzaldehyde oxime (0.285 g, 1.46 mmol) was dissolved in trifluoroacetic acid (3 mL) and cooled to 0° C. Zinc dust (0.477 g, 7.30 mmol) was slowly added and the reaction mixture was warmed to rt and stirred at rt for 1 h, then 2 mL of H₂O was added and TFA was removed under reduced pressure. The solution was brought to pH=8-9 by adding an aqueous solution of 2N NaOH, extracted by DCM (3×) and the combined organic layers were washed with brine, dried over MgSO₄, filtered and concentrated under reduced pressure to give the title compound (0.190 g, 71.8%), which was used in the next step without further purification. M.S. [M+H]⁺ 181.97 (ESI).

### Intermediate 135

### 2-ethoxy-4-methoxybenzaldehyde

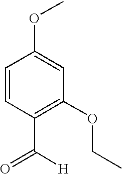

Following a procedure similar to that described for the preparation of intermediate 29, to a solution of 2-hydroxy-4-methoxybenzaldehyde (0.39 g, 2.56 mmol) in DMF (6.41 mL) was added potassium carbonate (0.531 g, 3.84 mmol) followed by iodoethane (0.207 mL, 2.56 mmol) at rt. The reaction mixture was stirred at 50° C. for 18 h. Water was added and extracted with EtOAc (2×). The combined organic phases were dried (MgSO₄), filtered and evaporated under reduced pressure to give the title compound, which was used without further purification. ¹H NMR (400 MHz, CDCl₃) δ ppm 1.48 (t, J=6.84 Hz, 3H), 3.87 (s, 3H), 4.13 (q, J=7.03 Hz, 2H), 6.44 (d, J=2.34 Hz, 1H), 6.54 (dd, J=8.79, 2.15 Hz, 1H), 7.82 (d, J=8.59 Hz, 1H), 10.34 (s, 1H).

### Intermediate 136

### 1-(2-ethoxy-4-methoxyphenyl)-N-methylmethanamine

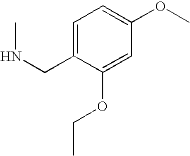

Following a procedure similar to that described for the preparation of Intermediate 133, starting from 2-ethoxy-4-methoxybenzaldehyde (Intermediate 135), the title compound (0.190 g, 97%) was obtained as an oil, which was used without further purification. MS [M+H]⁺ 195.98 (ESI).

### Intermediate 137

### (4-isopropoxy-2-methoxyphenyl)methanamine

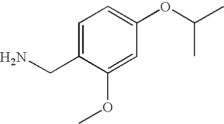

Following a procedure similar to that described in the preparation of Intermediate 40, isopropoxy-2-methoxybenzaldehyde (Intermediate 38) was convert to (S,E)-N-(4-isopropoxy-2-methoxybenzylidene)-2-methylpropane-2-sulfinamide (0.403 g, 88%) after purification by silica gel chromatography (0-10% MeOH/DCM). MS [M+H]⁺ 298.01 (ESI). To a solution of (S,E)-N-(4-isopropoxy-2-methoxybenzylidene)-2-methylpropane-2-sulfinamide (0.4 g, 1.34 mmol) in MeOH (5 mL) was added sodium tetrahydroborate (0.153 g, 4.03 mmol) at 0° C. The reaction mixture was allowed to warm to rt and was stirred for 18 h. The solvent was evaporated under reduced pressure, water was added and the mixture was extracted with DCM (3×). The organic layers were combined, dried over Na₂SO₄, filtered and concentrated under reduced pressure to give (S)—N-(4-isopropoxy-2-methoxybenzylidene)-2-methylpropane-2-sulfinamide, which was used in next step without further purification. MS [M+H]⁺ 300.02 (ESI). Following a procedure similar to that described in the preparation of Intermediate 40, the above sulfinamide was converted to the title compound (0.345 g) as its hydrochloride salt, which was used for the next step without further purification. MS [M+H]⁺ 195.98 (ESI).

### Intermediate 138

### 3-fluoro-4-(methoxymethyl)benzaldehyde

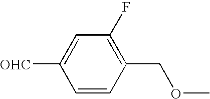

A suspension of (4-bromo-2-fluorophenyl)methanol (2.60 g, 12.68 mmol), dicyanozinc (1.042 g, 8.88 mmol) and Pd(Ph₃P)₄ (0.733 g, 0.63 mmol) in DMF (10 mL) was heated in a microwave reactor at 160° C. during 5 minutes. The reaction mixture was filtered on diatomaceous earth and the filtrate was concentrated. The residue was purified by silica gel chromatography (gradient 10-80% EtOAc in heptane) to provide 3-fluoro-4-(hydroxymethyl)benzonitrile (0.940 g, 49.0%) as a solid. ¹H NMR (400 MHz, CDCl₃) δ ppm 1.92-2.01 (m, 1H), 4.85 (d, J=6.25 Hz, 2H), 7.35 (dd, J=9.37, 1.56 Hz, 1H), 7.50 (d, J=7.81 Hz, 1H), 7.65 (t, J=7.62 Hz, 1H). M.S. 152.0 (ESI)(M+H)⁺. 3-fluoro-4-(hydroxymethyl)benzonitrile (464 mg, 3.07 mmol) was dissolved in THF (10 mL) followed by addition of sodium hydride (60% suspension in oil) (147 mg, 3.68 mmol). The suspension was stirred at rt during 10 minutes then methyl iodide (0.384 mL, 6.14 mmol) was added. The mixture was stirred at rt for 2 h then hydrolyzed and concentrated. The residue was purified by silica gel chromatography (gradient 7-60% EtOAc in heptane) to give 3-fluoro-4-(methoxymethyl)benzonitrile (363 mg, 71.6%) as a solid. ¹H NMR (400 MHz, CDCl₃) δ ppm 3.47 (s, 3H), 4.58 (s, 2H), 7.35 (dd, J=9.37, 1.56 Hz, 1H), 7.43-7.52 (m, 1H), 7.59 (t, J=7.62 Hz, 1H). To a solution of 3-fluoro-4-(methoxymethyl)benzonitrile (360 mg, 2.18 mmol) in CH₂Cl₂ (10 mL) at 0° C. under N₂ was slowly added diisobutylaluminum hydride (1M solution in toluene) (2.83 mL, 2.83 mmol). The reaction mixture was allowed to reach rt and stirred during 16 h. After addition of 5 mL of HCl 10%, the mixture was heated at refluxed for 30 minutes and filtered on diatomaceous earth. The aqueous phase from the filtrate was extracted with CH₂Cl₂. The combined organic phases were dried over MgSO₄, filtered, and concentrated under reduced pressure. The residue was purified by silica gel chromatography (gradient 7-60% EtOAc in heptane) to provide the title compound (162 mg, 44.2%) as an oil. ¹H NMR (400 MHz, CDCl₃) δ ppm 3.48 (s, 3H), 4.60 (s, 2H), 7.56 (d, J=9.37 Hz, 1H), 7.62-7.72 (m, 2H), 9.99 (d, J=1.95 Hz, 1H).

### Intermediate 139

### (R)-1-(3-fluoro-4-(methoxymethyl)phenyl)ethanamine

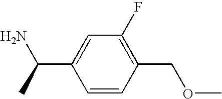

Following a procedure similar to that described in the preparation of Intermediate 40, starting from 3-fluoro-4-(methoxymethyl)benzaldehyde (Intermediate 138) and after purification by silica gel chromatography (gradient 10-100% MeOH in EtOAc), the title compound was obtained as an oil. ¹H NMR (400 MHz, CDCl₃) δ ppm 1.71 (d, J=6.64 Hz, 3H), 3.42 (s, 3H), 4.48 (s, 2H), 4.83 (s, 1H), 7.32 (d, J=9.37 Hz, 2H), 7.42 (t, J=7.62 Hz, 1H), 8.83 (br. s., 2H).

### Intermediate 140

### 1-(7-chloroisoquinolin-3-yl)-N-methylmethanamine

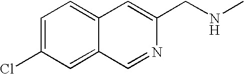

A solution of 2-amino-3-(4-chlorophenyl)propanoic acid (1.0 g, 5.01 mmol) and 37% aqueous paraformaldehyde (3155 mg) in HBr (20 mL) was stirred in microwave at 120° C. for 5 minutes. The solvent was removed, the residue was refluxed in methanol (50 mL) and 0.5 mL of concentrated HCl overnight. The solvent was removed to give methyl 7-chloro-1,2,3,4-tetrahydroisoquinoline-3-carboxylate, which was used in the next step without further purification. M.S. 225.92. (ESI) (MH⁺). A solution of methyl 7-chloro-1,2,3,4-tetrahydroisoquinoline-3-carboxylate (500 mg, 2.22 mmol) in 50 mL of DMF and 1 mL of DIPEA was stirred at 100° C. for 12 h. The solvent was removed to give a residue, which was purified by silica gel chromatography, eluated with heptane/ethyl acetate/methanol (4:4:1) to give methyl 7-chloroisoquinoline-3-carboxylate (455 mg, 93%). M.S. 221.89. (ESI) (MH⁺). A solution of methyl 7-chloroisoquinoline-3-carboxylate (490 mg, 2.21 mmol) in 30 mL of THF was added aluminum(III) lithium hydride (490 mg, 12.91 mmol) at −78° C. The solution was stirred at −78° C. for 2 h. To the reaction solution was added 20% NaOH (2 mL), the product was extracted with ethyl acetate and washed with water and brine. The crude was purified by silica gel column, eluated with heptane/ethyl acetate/methanol (4:4:0.1) to give (7-chloroisoquinolin-3-yl)methanol (175 mg, 40.9%). M.S. 193.94. (ESI) (MH⁺). To a solution of (7-chloroisoquinolin-3-yl)methanol (200 mg, 1.03 mmol) in 10 mL of CH₂Cl₂ was added sulfurous dichloride (184 mg, 1.55 mmol) at 0° C. The reaction was stirred at 0° C. for 10 minutes. Then, it was stirred at rt for 1 h. The solvent was removed to give 7-chloro-3-(chloromethyl)isoquinoline, which was used in the next step without further purification. M.S. 213.87. (ESI) (MH⁺). A solution of 7-chloro-3-(chloromethyl)isoquinoline (0.218 g, 1.03 mmol) in 10 mL of acetonitrile was added to methanamine (0.80 g, 10.30 mmol) at 0° C. The reaction mixture was stirred at 0° C. for 20 minutes, then at rt for 1 h. The solvent was removed to give a residue, which was purified by silica gel chromatography (eluted with acetyl acetate/methanol (10:1 to 1:1) to give the title compound (113 mg, 53.1%) as a solid. M.S. 207.99. (ESI) (MH⁺)

### Intermediate 141

### N-methyl-1-(6-methylisoquinolin-3-yl)methanamine

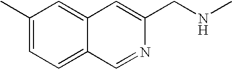

Following a procedure similar to that described for the preparation of Intermediate 140 and after purification by silica gel chromatography (ethyl acetate/methanol, 10:1 to 1:1), the title compound (0.215 g, 12% over 5 steps) was obtained as a solid. M.S. 205.97. (ESI) (MH⁺).

### Intermediate 142

### 1-(quinolin-3-yl)ethanamine hydrochloride

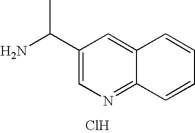

Following a procedure similar to that described for the preparation of Intermediate 40 and starting from (S)-2-methylpropane-2-sulfinamde and quinoline-3-carbaldehyde, the title compound (336 mg, 37% over 3 steps) was obtained as its HCl salt, which was a mixture of two enantiomers (note: enriched R-isomer) and was used in the next step without further purification. M.S. 173.2 (ESI) (MH⁺).

### Intermediate 143

### N-methyl-1-(quinolin-3-yl)methanamine

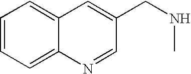

Following a procedure similar to that described for the preparation of Intermediate 63, the title compound (626 mg, 92%) was obtained as its TFA salt, which was used in the next step without further purification. M.S. 173.2 (ESI) (MH⁺).

### Intermediate 144

### N-methyl-1-(2-methylquinolin-3-yl)methenamine hydrochloride

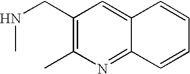

Following a procedure similar to that described for the preparation of Intermediate 65 and starting from (2-methylquinolin-3-yl)methanamine (Intermediate 64), the title compound (a 1.5 mmol) was used in the next step without further purification. M.S. 187.2 (ESI) (MH⁺).

### Intermediate 145

### 1-(6-chloroisoquinolin-3-yl)-N-methylmethanamine

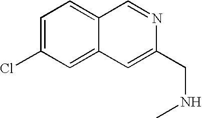

Following a procedure similar to that described for the preparation of Intermediate 140 and after purification silica gel chromatography (10-50% methanol/ethyl acetate), the title compound (198 mg) was obtained as a solid. M.S. 206.91 (ESI) (MH⁺).

### Intermediate 146

### 1-(6-fluoroisoquinolin-3-yl)-N-methylmethanamine

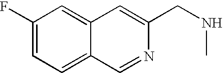

Following a procedure similar to that described for the preparation of Intermediate 140, the title compound (277 mg, 25% over 5 steps) was obtained as a solid, which was used in the next step without further purification. M.S. 206.91 (ESI) (MH⁺).

### Intermediate 147

### 1-(7-fluoroisoquinolin-3-yl)-N-methylmethanamine

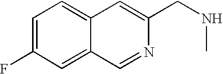

Following a procedure similar to that described for the preparation of Intermediate 140 and after purification by silica gel chromatography (ethyl acetate/methanol 4:1 to 1:1), the title compound (105 mg, 10% over 5 steps) was obtained as a solid. M.S. 190.96 (ESI) (MH⁺).

### Intermediate 148

### (R)-4-(1-aminoethyl)benzonitrile hydrochloride

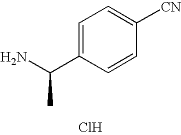

Following a procedure similar to that described in the preparation of Intermediate 40, the title compound (0.355 g, 26% over 3 steps) was obtained as hydrochloride salt. ¹H NMR (400 MHz, DMSO-d₆) δ ppm 1.46 (d, J=7.03 Hz, 3H), 4.46 (quin, J=5.92, 5.66 Hz, 1H), 7.70 (d, J=8.59 Hz, 2H), 7.87 (d, J=8.20 Hz, 2H).

### Intermediate 149

### 1-(quinolin-3-yl)ethanamine hydrochloride (Enantiomer 1) and

### Intermediate 150

### 1-(quinolin-3-yl)ethanamine hydrochloride (Enantiomer 2)

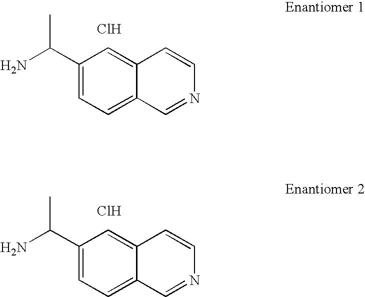

Following a procedure similar to that described for Intermediate 40 and starting from (S)-2-methylpropane-2-sulfinamde and isoquinoline-6-carbaldehyde, after purification by silica gel chromatography (10% methanol/ethyl acetate), N-(1-(isoquinolin-6-yl)ethyl)-2-methylpropane-2-sulfinamide (1.10 g, 80% over 2 steps, mixture of two diastereomers) was obtained as an oil. M.S. 277.02. (ESI) (MH⁺).

To a solution of N-(1-(isoquinolin-6-yl)ethyl)-2-methylpropane-2-sulfinamide (300 mg, 1.09 mmol) in THF (10 mL) at 0° C. was added sodium hydride (174 mg, 4.34 mmol, 60% in oil). The reaction mixture was stirred at 0° C. for 15 minutes, iodoethane (339 mg, 2.17 mmol) was then added. The reaction mixture was stirred at 0° C. for an additional 30 minutes. The reaction mixture was warmed to rt and stirred at rt for 2 h, concentrated under reduced pressure and the residue was purified by silica gel chromatography (20-80% ethyl acetate in heptane) to give two diastereomers: Diastereomer 1: N-ethyl-N-(1-(isoquinolin-6-yl)ethyl)-2-methylpropane-2-sulfinamide (237 mg, 71.7%). M.S. 305.30. (ESI) (MH⁺). Diastereomer 2: N-ethyl-N-(1-(isoquinolin-6-yl)ethyl)-2-methylpropane-2-sulfinamide (80 mg, 24.21%). M.S. 305.31. (ESI) (MH⁺).

A solution of N-ethyl-N-(1-(isoquinolin-6-yl)ethyl)-2-methylpropane-2-sulfinamide (Diastereomer 1) (150 mg, 0.50 mmol) in 20 mL of methanol and 10 mL of 10% HCl aqueous solution was stirred at 40° C. for 30 minutes. The solution was concentrated under reduced pressure to yield 1-(quinolin-3-yl)ethanamine hydrochloride (Enantiomer 1), which was used in the next step without further purification. M.S. 201.28. (ESI) (MH⁺).

A solution of N-ethyl-N-(1-(isoquinolin-6-yl)ethyl)-2-methylpropane-2-sulfinamide (Diastereomer 2) (25.0 mg, 0.082 mmol) in 10 mL of methanol and 5 mL of 10% HCl aqueous solution was stirred at 40° C. for 30 minutes. The solution was concentrated under reduced pressure to yield 1-(quinolin-3-yl)ethanamine hydrochloride (Enantiomer 2), which was used in the next step without further purification. M.S. 201.22. (ESI) (MH⁺).

### Intermediate 151

### 1-(3-Methyl-piperazin-1-yl)-ethanone hydrochloride

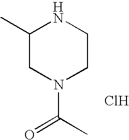

2-Methyl-piperazine-1-carboxylic acid tert-butyl ester (1.0 g, 5.0 mmol) was dissolved in anhydrous CH₂Cl₂ (15 mL) under N₂. Acetic anhydride (0.52 mL, 5.0 mmol) was added and the solution was refluxed for 2 h, cooled to rt and concentrated under reduced pressure. Toluene was added to the residue and concentrated under reduced pressure to ensure complete removal of excess acetic anhydride. The title compound (1.2 g, 100%) was obtained as an oil, which was used without further purification. To a solution of 4-acetyl-2-methyl-piperazine-1-carboxylic acid tert-butyl ester (1.2 g, 5 mmol) in CH₂Cl₂ (12 mL) was added a solution of 4M HCl in dioxane (5 mL, 20 mmol) under N₂. The reaction mixture was stirred at rt for 4 h, the solid was filtered and washed with CH₂Cl₂ (5 mL) to give the title compound (as its HCl salt) as a white solid (0.83 g, 93%), which was used without further purification. ¹H NMR (400 MHz, CD₃OD) δ ppm 1.35 (t, J=6.88 Hz, 3H), 2.15 (s, 3H), 2.83 (d, J=9.95 Hz, 1H), 2.96-3.11 (m, 1H), 3.12-3.28 (m, 2H), 3.34-3.47 (m, 2H), 4.07 (t, J=12.00 Hz, 1H), 4.42-4.63 (m, 1H).

### Intermediate 152

### N-methyl-1-(7-methylisoquinolin-3-yl)methanamine

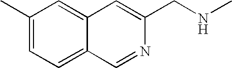

Following a procedure similar to that described for the preparation of Intermediate 140 and after purification by silica gel chromatography (ethyl acetate:methanol 1:1), the title compound was obtained as a solid (23 mg, 8% over 5 steps). M.S. 186.97. (ESI) (MH⁺).

### Intermediate 153

### (R)-1-(2,4-diethoxyphenyl)ethanamine hydrochloride

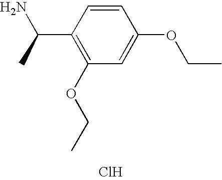

To a solution of 2,4-dihydroxybenzaldehyde (1 g, 7.24 mmol) in DMF (20 ml) was added potassium carbonate (3.00 g, 21.72 mmol) followed by iodoethane (1.753 ml, 21.72 mmol) at rt. The reaction mixture was stirred at 50° C. for 18 h. Water was added and the mixture was extracted with EtOAc (2×). The organic layer was dried (MgSO₄), filtered and concentrated under reduced pressure to yield 2,4-diethoxybenzaldehyde, which was used without further purification. ¹H NMR (400 MHz, CD₃OD), δ ppm 1.40-1.51 (m, 6H), 4.05-4.16 (m, 4H), 6.42 (d, J=2.34 Hz, 1H), 6.52 (dd, J=8.79, 2.15 Hz, 1H), 7.80 (d, J=8.98 Hz, 1H), 10.32 (s, 1H). Following a procedure similar to that described for the preparation of Intermediate 40 and starting from 2,4-diethoxybenzaldehyde, the title compound (40 mg, 4.9% over 3 steps) was obtained as a solid. MS [M+H]⁺ 209.99 (ESI).

### Intermediate 154

### (R)-1-(2,2-difluorobenzo[d][1,3]dioxol-5-yl)ethanamine

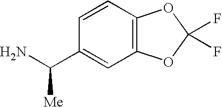

Following a procedure similar to that described for the preparation of Intermediate 40, the title compound (328 mg, 29% over 3 steps) was obtained. [α]_{D}=+3.6 (c=0.01, MeOH). ¹H NMR (400 MHz, CD₃OD) δ ppm 1.61 (d, J=7.03 Hz, 3H), 4.49 (q, J=6.64 Hz, 1H), 7.27 (s, 2H), 7.36 (s, 1H).

### Intermediate 155

### 1-(2,2-difluorobenzo[d][1,3]dioxol-5-yl)-N-methylmethanamine

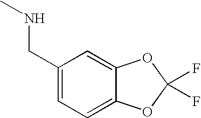

To a solution of 2,2-difluorobenzo[d][1,3]dioxole-5-carbaldehyde (1 g, 5.37 mmol) in ethanol (20 mL) at 0° C. was added sodium borohydride (0.305 g, 8.06 mmol). The reaction mixture was stirred at rt for 16 h. Water was added and the mixture was extracted with DCM (3×), washed with water, dried (MgSO₄), filtered and concentrated under reduced pressure. The residue was purified by silica gel chromatography (0% to 75% EtOAc in heptane) to give (2,2-difluorobenzo[d][1,3]dioxol-5-yl)methanol (0.802 g, 79%) as an oil. ¹H NMR (400 MHz, CDCl₃) δ ppm 1.72-1.86 (m, 1H), 4.69 (d, J=3.91 Hz, 2H), 6.96-7.10 (m, 2H), 7.13 (s, 1H). To a solution of the above intermediate (2,2-difluorobenzo[d][1,3]dioxol-5-yl)methanol (802 mg, 4.26 mmol) in 50 mL of CH₂Cl₂ at 0° C. was added sulfurous dichloride (0.466 mL, 6.39 mmol). The reaction mixture was stirred at rt for 1 h. Additional sulfurous dichloride (0.466 mL, 6.39 mmol) was added and the reaction mixture was stirred at rt for 16 h. After concentration under reduced pressure, the residue was purified by silica gel chromatography (0-40% EtOAc in heptane) to give 5-(chloromethyl)-2,2-difluorobenzo[d][1,3]dioxole (336 mg, 38.2%) as an oil. ¹H NMR (400 MHz, CDCl₃) δ ppm 4.54 (s, 2H), 6.97-7.03 (m, 1H), 7.04-7.09 (m, 1H), 7.11 (s, 1H). To a solution of the above intermediate 5-(chloromethyl)-2,2-difluorobenzo[d][1,3]dioxole (0.336 g, 1.63 mmol) in acetonitrile (6 mL) at 0° C. was added a 40% aqueous solution of methanamine (1.4 mL, 16.27 mmol). The reaction mixture was stirred at rt for 3 h. The solvent was evaporated under reduced pressure and the residue was dissolved in water (50 mL) and DIPEA (5 mL), then concentrated under reduced pressure to give the title compound (0.266 g, 81%) as a solid, which was used to the next step without purification. M.S. (found): 202.17 (ESI) (MH⁺).

### Intermediate 156

### 1-ethylpiperazin-2-one

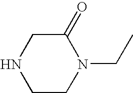

Sodium hydride (0.210 g, 5.24 mmol) was added to a solution of tert-butyl 3-oxopiperazine-1-carboxylate (1 g, 4.99 mmol) in DMF (10 mL) at rt and the reaction mixture was stirred for 15 minutes. Iodoethane (0.639 mL, 7.99 mmol) was added slowly and the reaction mixture was stirred at rt for 2 h. Water was added and the mixture was extracted with EtOAc (3×). The organic extracts were washed with water (3×) and brine, dried with MgSO₄ and concentrated under reduced pressure to give tert-butyl 4-ethyl-3-oxopiperazine-1-carboxylate (0.955 g, 84%) as an oil. M.S. (found): 229.25 (ESI) (MH⁺). To a solution of the above intermediate tert-butyl 4-ethyl-3-oxopiperazine-1-carboxylate (955 mg, 4.18 mmol) was added a solution of TFA in DCM (1:1 v/v, 10 mL). The reaction mixture was stirred at rt for 40 minutes, concentrated under reduced pressure to give the title compound (1.61 g) as its TFA salt. ¹H NMR (400 MHz, CD₃OD) [symbol] ppm 1.18 (t, J=7.23 Hz, 3H), 3.44-3.57 (m, 4H), 3.63 (t, J=5.66 Hz, 2H), 3.82 (s, 2H).

### Intermediate 157

### 1-(2,2,2-trifluoroethyl)piperazin-2-one

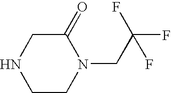

Sodium hydride (110 mg, 2.75 mmol) was added to a solution of tert-butyl 3-oxopiperazine-1-carboxylate (500 mg, 2.50 mmol) in DMF (15 mL) at 0° C. and the reaction mixture was stirred at rt for 30 minutes. 2,2,2-trifluoroethyl trifluoromethanesulfonate (1.7 mL, 12.49 mmol) was added and the reaction mixture was stirred for 16 h, concentrated under reduced pressure and the residue was diluted with 1N aq. NaOH and extracted with EtOAc (3×), washed with brine, dried (MgSO₄), filtered and concentrated under reduced pressure, the residue was purified by silica gel chromatography (0-100% EtOAc in Heptane) to give tert-butyl 3-oxo-4-(2,2,2-trifluoroethyl)piperazine-1-carboxylate (228 mg, 32.3%) as an oil. ¹H NMR (400 MHz, CDCl₃) δ ppm 1.48 (s, 9H), 3.52 (t, J=5.27 Hz, 2H), 3.69 (t, J=5.27 Hz, 2H), 4.07 (q, J=8.98 Hz, 2H), 4.17 (s, 2H). To a solution of the above intermediate tert-butyl 3-oxo-4-(2,2,2-trifluoroethyl)piperazine-1-carboxylate (258 mg, 0.91 mmol) was added a solution of TFA in DCM (1:1 v/v, 10 mL). The mixture was stirred at rt for 40 minutes, concentrated under reduced pressure to give the title compound (456 mg, quantitative yield) as its TFA salt. ¹H NMR (400 MHz, CD₃OD) δ ppm 3.48-3.65 (m, 2H), 3.72-3.85 (m, 2H), 3.95 (s, 2H), 4.25 (q, J=9.11 Hz, 2H).

### Examples of the Invention

### Example 1

### 2-(4-acetylpiperazin-1-yl)-6-isopropyl-4-(1,2,3,4-tetrahydronaphthalen-1-ylamino)-5,6-dihydro-7H-pyrrolo[3,4-d]pyrimidin-7-one

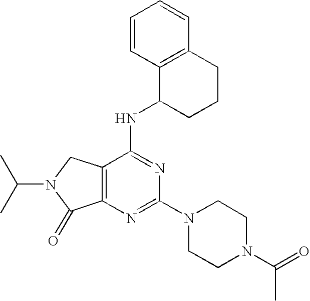

Following a procedure similar to that described in General Procedure 1 and after purification by silica gel chromatography (gradient 0-40% methanol: ethyl acetate) followed by preparative LCMS (gradient 35-55% CH₃CN in H₂O containing 0.1% trifluoroacetic acid), the title compound was obtained as its TFA salt. HPLC: k′ 12.24; Purity: >94% (215 nm), >95% (254 nm), >94% (280 nm); Rₜ: 1.72 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.64 Hz, 6H), 1.78-2.06 (m, 4H), 2.07-2.19 (m, 3H), 2.73-2.99 (m, 2H), 3.67-3.76 (m, 4H), 3.82-3.88 (m, 2H), 3.89-3.95 (m, 2H), 4.28 (s, 2H), 4.40-4.52 (m, 1H), 5.57 (t, J=5.96 Hz, 1H), 7.08-7.25 (m, 4H). M.S. (calcd): 449.2 (MH⁺), M.S. (found): 449.3 (ESI) (MH⁺).

### Example 2

### ethyl 3-{[2-(4-acetylpiperazin-1-yl)-6-isopropyl-7-oxo-6,7-dihydro-5H-pyrrolo[3,4-d]pyrimidin-4-yl]amino}-2-phenylpropanoate

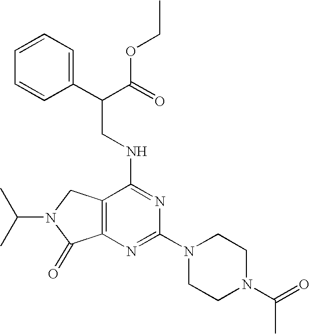

Following a procedure similar to that described in General Procedure 1 and after purification by silica gel chromatography (gradient 0-40% methanol:ethyl acetate) followed by preparative LCMS (gradient 35-55% CH₃CN in H₂O containing 0.1% trifluoroacetic acid), the title compound was obtained as its TFA salt. HPLC: k′ 11.66; Purity: >96% (215 nm), >97% (254 nm), >96% (280 nm); Rₜ: 1.65 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.15 (t, J=7.13 Hz, 3H), 1.28 (d, J=6.64 Hz, 6H), 2.15 (s, 3H), 3.67-3.76 (m, 4H), 3.81-3.95 (m, 6H), 4.04-4.17 (m, 4H), 4.22 (s, 2H), 4.40-4.50 (m, 1H), 7.24-7.39 (m, 5H). M.S. (calcd): 495.2 (MH⁺), M.S. (found): 495.3 (ESI) (MH⁺)

### Example 3

### 2-(4-acetylpiperazin-1-yl)-4-[benzyl(tetrahydrofuran-2-ylmethyl)amino]-6-isopropyl-5,6-dihydro-7H-pyrrolo[3,4-d]pyrimidin-7-one

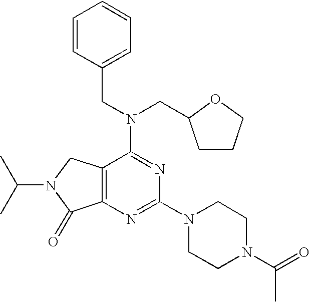

Following a procedure similar to that described in General Procedure 1 and after purification by silica gel chromatography (gradient 0-40% methanol: ethyl acetate) followed by preparative LCMS (gradient 45-65% CH₃CN in H₂O containing 0.1% trifluoroacetic acid), the title compound was obtained as its TFA salt. HPLC: k′ 13.07; Purity: >92% (215 nm), >92% (254 nm), >92% (280 nm); Rₜ: 1.83 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.21-1.30 (m, 6H), 1.40 (m, 2H), 1.91-1.97 (m, 2H), 2.05-2.15 (m, 3H), 3.53-3.64 (m, 6H), 3.69-3.93 (m, 9H), 4.22-4.29 (m, 2H), 4.40-4.49 (m, 1H), 7.22-7.29 (m, 3H), 7.30-7.37 (m, 2H). M.S. (calcd): 493.2 (MH⁺), M.S. (found): 493.3 (ESI) (MH⁺).

### Example 4

### 2-(4-acetylpiperazin-1-yl)-4-[cyclopentyl(4-fluorobenzyl)amino]-6-isopropyl-5,6-dihydro-7H-pyrrolo[3,4-d]pyrimidin-7-one

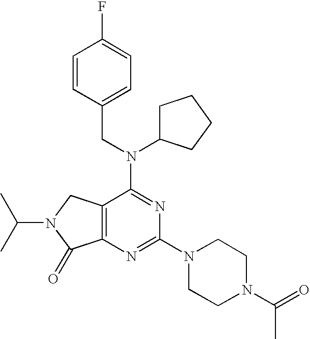

To a solution of 2,4-dichloro-6-isopropyl-5,6-dihydropyrrolo[3,4-d]pyrimidine-7-one (Intermediate 3, 50 mg, 0.203 mmol) in dichloroethane (2.0 mL) was added N-(4-fluorobenzyl)cyclopentanamine (41 mg, 0.213 mmol) followed by triethylamine (57 μL, 0.406 mmol) at rt. The mixture was heated in a microwave at 225° C. for 20 minutes, and then concentrated in vacuo. The resulting residue was combined with 1-acetylpiperazine (52 mg, 0.406 mmol) in n-butanol (3 mL) and heated at 130° C. for 12 h. The residue was purified by silica gel chromatography (gradient 0-40% methanol: ethyl acetate) followed by preparative LCMS (gradient 45-65% CH₃CN in H₂O containing 0.1% trifluoroacetic acid) to give the title compound as its TFA salt. HPLC: k′ 15.12; Purity: >90% (215 nm), >93% (254 nm), >93% (280 nm); Rₜ: 1.72 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19-1.30 (m, 8H), 1.40 (m, 2H), 1.88-1.97 (m, 2H), 2.05-2.15 (m, 5H), 3.53-3.64 (m, 4H), 3.69-3.93 (m, 8H), 4.18-4.29 (m, 1H), 4.41-4.50 (m, 1H), 7.22-7.29 (m, 2H), 7.30-7.37 (m, 2H). M.S. (calcd): 495.3 (MH⁺), M.S. (found): 495.3 (ESI) (MH⁺).

### Example 5

### 2-(4-acetylpiperazin-1-yl)-4-{[1-(4-tert-butylphenyl)ethyl]amino}-6-isopropyl-5,6-dihydro-7H-pyrrolo[3,4-d]pyrimidin-7-one

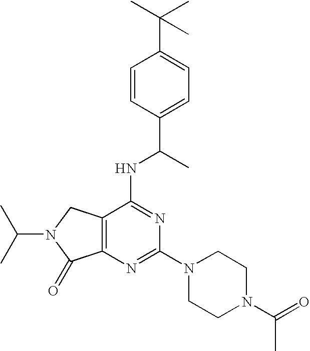

Following a procedure similar to that described in General Procedure 1 and after purification by silica gel chromatography (gradient 0-40% methanol: ethyl acetate) followed by preparative LCMS (gradient 45-65% CH₃CN in H₂O containing 0.1% trifluoroacetic acid), the title compound was obtained as its TFA salt. HPLC: k′ 15.18; Purity: >96% (215 nm), >99% (254 nm), >99% (280 nm); Rₜ: 2.10 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.26 (s, 9H), 1.28-1.32 (m, 6H), 1.58 (d, J=7.03 Hz, 3H), 2.11 (s, 3H), 3.47-3.88 (m, 8H), 4.34 (s, 2H), 4.40-4.51 (m, 1H), 5.29 (q, J=6.84 Hz, 1H), 7.25-7.40 (m, 4H). M.S. (calcd): 479.3 (MH⁺), M.S. (found): 479.3 (ESI) (MH⁺).

### Example 6

### 2-(4-acetylpiperazin-1-yl)-4-{[1-(4-isobutylphenyl)ethyl]amino}-6-isopropyl-5,6-dihydro-7H-pyrrolo[3,4-d]pyrimidin-7-one

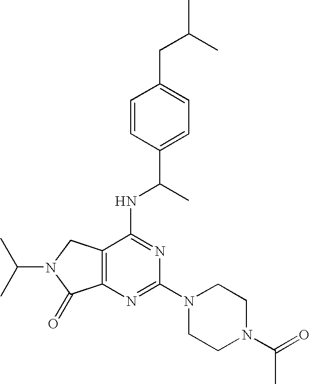

Following a procedure similar to that described in General Procedure 1 and after purification by silica gel chromatography (gradient 0-40% methanol: ethyl acetate) followed by preparative LCMS (gradient 45-65% CH₃CN in H₂O containing 0.1% trifluoroacetic acid), the title compound was obtained as its TFA salt. HPLC: k′ 15.63; Purity: >94% (215 nm), >96% (254 nm), >94% (280 nm); Rₜ: 2.16 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 0.82-0.87 (m, 6H), 1.27-1.34 (m, 7H), 1.52-1.61 (m, 3H), 1.74-1.84 (m, 1H), 2.08-2.13 (m, 3H), 2.42 (d, J=7.23 Hz, 2H), 3.50-3.67 (m, 4H), 3.69-3.86 (m, 4H), 4.34 (s, 2H), 4.42-4.50 (m, 1H), 7.10 (d, J=8.20 Hz, 2H), 7.27 (d, J=8.01 Hz, 2H). M.S. (calcd): 479.3 (MH⁺), M.S. (found): 479.3 (ESI) (MH⁺).

### Example 7

### 2-{[2-(dimethylamino)ethyl]amino}-6-isopropyl-4-{[(4-methylphenyl)(phenyl)methyl]amino}-5,6-dihydro-7H-pyrrolo[3,4-d]pyrimidin-7-one

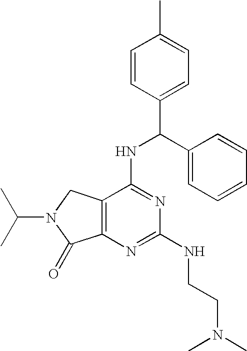

Following a procedure similar to that described in General Procedure 1 and after purification by silica gel chromatography (gradient 0-50% methanol:ethyl acetate) followed by preparative LCMS (gradient 45-65% CH₃CN in H₂O containing 0.1% ammonium carbonate), the title compound was obtained, which was treated with TFA and lyophilized to give the title compound as its TFA salt. HPLC: k′ 11.45; Purity: >98% (215 nm), >99% (254 nm), >99% (280 nm); Rₜ: 1.62 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.21 (d, J=6.84 Hz, 6H), 2.23 (s, 3H), 2.67 (s, 6H), 3.00 (s, 2H), 3.51-3.59 (m, 2H), 4.22 (s, 2H), 4.35-4.46 (m, 1H), 6.45 (s, 1H), 7.05-7.13 (m, 4H), 7.14-7.31 (m, 5H). M.S. (calcd): 459.3 (MH⁺), M.S. (found): 459.2 (ESI) (MH⁺).

### Example 8

### 2-(4-acetylpiperazin-1-yl)-4-{[2-(4-chlorophenyl)propyl]amino}-6-isopropyl-5,6-dihydro-7H-pyrrolo[3,4-d]pyrimidin-7-one

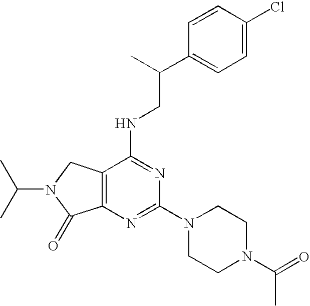

Following a procedure similar to that described in General Procedure 1 and after purification by silica gel chromatography (gradient 0-40% methanol: ethyl acetate) followed by preparative LCMS (gradient 35-55% CH₃CN in H₂O containing 0.1% trifluoroacetic acid), the title compound was obtained as its TFA salt. HPLC: k′ 1.79; Purity: >98% (215 nm), >99% (254 nm), >99% (280 nm); Rₜ: 1.79 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.27 (d, J=6.64 Hz, 6H), 1.30 (d, J=7.03 Hz, 3H), 2.14 (s, 3H), 3.16 (q, J=7.03 Hz, 1H), 3.56-3.66 (m, 6H), 3.77-3.83 (m, 2H), 3.84-3.90 (m, 2H), 4.11 (d, J=2.15 Hz, 2H), 4.43-4.53 (m, 1H), 7.19-7.29 (m, 4H). M.S. (calcd): 471.2 (MH⁺), M.S. (found): 471.3 (ESI) (MH⁺).

### Example 9

### 4-{[(4-chlorophenyl)(phenyl)methyl]amino}-2-{[2-(dimethylamino)ethyl]amino}-6-isopropyl-5,6-dihydro-7H-pyrrolo[3,4-d]pyrimidin-7-one

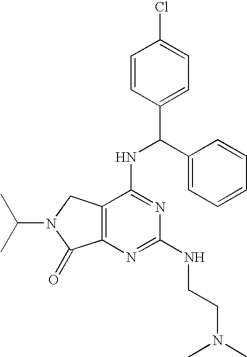

Following a procedure similar to that described in General Procedure 1 and after purification by silica gel chromatography (gradient 0-50% methanol: ethyl acetate) followed by preparative LCMS (gradient 35-55% CH₃CN in H₂O containing 0.1% trifluoroacetic acid), the title compound was obtained as its TFA salt. HPLC: k′ 12.00; Purity: >99% (215 nm), >98% (254 nm), >99% (280 nm); Rₜ: 1.69 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.64 Hz, 6H), 2.71 (s, 6H), 2.87-3.17 (m, 2H), 3.58-3.83 (m, 2H), 4.36 (s, 2H), 4.40-4.56 (m, 1H), 6.54 (s, 1H), 7.12-7.51 (m, 9H). M.S. (calcd): 479.2 (MH⁺), M.S. (found): 479.3 (ESI) (MH⁺).

### Example 10

### 2-(4-acetylpiperazin-1-yl)-6-isopropyl-4-{[1-(4-isopropylphenyl)-2-methylpropyl]amino}-5,6-dihydro-7H-pyrrolo[3,4-d]pyrimidin-7-one

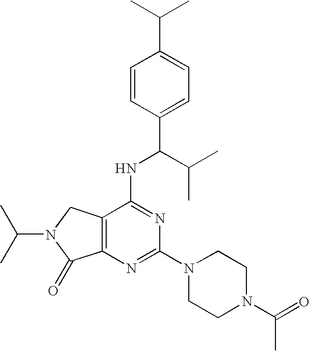

Following a procedure similar to that described in General Procedure 1 and after purification by silica gel chromatography (gradient 0-40% methanol: ethyl acetate) followed by preparative LCMS (gradient 45-65% CH₃CN in H₂O containing 0.1% trifluoroacetic acid), the title compound was obtained as its TFA salt. HPLC: k′ 16.22; Purity: >95% (215 nm), >96% (254 nm), >97% (280 nm); Rₜ: 2.24 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 0.76-0.80 (m, 3H), 1.12 (d, J=6.45 Hz, 3H), 1.19 (d, J=6.84 Hz, 6H), 1.28-1.34 (m, 6H), 2.14 (s, 3H), 2.16-2.24 (m, 1H), 2.77-2.92 (m, 1H), 3.56-3.91 (m, 10H), 4.41-4.49 (m, 1H), 4.78 (d, J=9.96 Hz, 1H), 7.15-7.20 (m, 2H), 7.24-7.30 (m, 2H). M.S. (calcd): 493.3 (MH⁺), M.S. (found): 493.3 (ESI) (MH⁺).

### Example 11

### 2-(4-acetylpiperazin-1-yl)-6-isopropyl-4-{[(1R)-1-(4-methoxyphenyl)ethyl]amino}-5,6-dihydro-7H-pyrrolo[3,4-d]pyrimidin-7-one

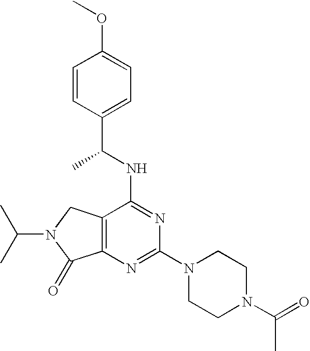

Following a procedure similar to that described in General Procedure 1 and after purification by silica gel chromatography (gradient 0-40% methanol: ethyl acetate) followed by preparative LCMS (gradient 35-55% CH₃CN in H₂O containing 0.1% trifluoroacetic acid), the title compound was obtained as its TFA salt. HPLC: k′ 10.60; Purity: >95% (215 nm), >97% (254 nm), >96% (280 nm); Rₜ: 1.51 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (dd, J=6.84, 2.15 Hz, 6H), 1.57 (d, J=7.03 Hz, 3H), 2.12 (s, 3H), 3.50-3.70 (m, 4H), 3.74 (s, 3H), 3.75-3.80 (m, 2H), 3.79-3.86 (m, 2H), 4.34 (s, 2H), 4.41-4.50 (m, 1H), 5.29 (q, J=6.71 Hz, 1H), 6.83-6.89 (m, 2H), 7.26-7.33 (m, 2H). M.S. (calcd): 453.2 (MH⁺), M.S. (found): 453.3 (ESI) (MH⁺).

### Example 12

### 2-(4-acetylpiperazin-1-yl)-4-{[2-(4-fluorophenyl)-1-methylethyl]amino}-6-isopropyl-5,6-dihydro-7H-pyrrolo[3,4-d]pyrimidin-7-one

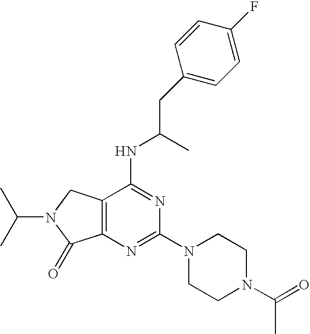

Following a procedure similar to that described in General Procedure 1 and after purification by silica gel chromatography (gradient 0-40% methanol: ethyl acetate) followed by preparative LCMS (gradient 35-55% CH₃CN in H₂O containing 0.1% trifluoroacetic acid), the title compound was obtained as its TFA salt. HPLC: k′ 6.11; Purity: >92% (215 nm), >99% (254 nm), >99% (280 nm); Rₜ: 1.64 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.26-1.33 (m, 9H), 2.14 (s, 3H), 2.88 (d, J=7.03 Hz, 2H), 3.63-3.72 (m, 4H), 3.74-3.89 (m, 4H), 4.24 (d, J=6.44 Hz, 2H), 4.39-4.50 (m, 1H), 4.56-4.68 (m, 1H), 6.90-7.01 (m, 2H), 7.17-7.25 (m, 2H). M.S. (calcd): 455.2 (MH⁺), M.S. (found): 455.3 (ESI) (MH⁺).

### Example 13

### 2-(4-acetylpiperazin-1-yl)-6-isopropyl-4-{[1-(3-phenyl-1,2,4-oxadiazol-5-yl)ethyl]amino}-5,6-dihydro-7H-pyrrolo[3,4-d]pyrimidin-7-one

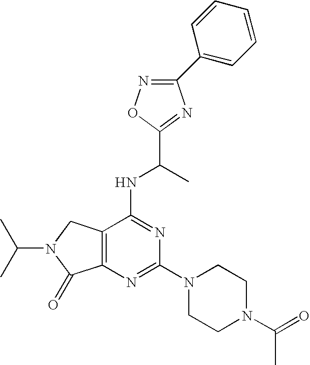

Following a procedure similar to that described in General Procedure 1 and after purification by silica gel chromatography (gradient 0-40% methanol:ethyl acetate) followed by preparative LCMS (gradient 35-55% CH₃CN in H₂O containing 0.1% trifluoroacetic acid), the title compound was obtained as its TFA salt. HPLC: k′ 6.52; Purity: >99% (215 nm), >99% (254 nm), >99% (280 nm); Rₜ: 1.73 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=6.84 Hz, 6H), 1.73-1.79 (m, 3H), 1.98 (s, 3H), 3.31-3.48 (m, 4H), 3.58-3.77 (m, 4H), 4.31 (d, J=4.30 Hz, 2H), 4.46-4.57 (m, 1H), 5.41-5.52 (m, 1H), 7.44-7.58 (m, 3H), 7.97-8.08 (m, 2H). M.S. (calcd): 491.3 (MH⁺), M.S. (found): 491.3 (ESI) (MH⁺).

### Example 14

### 2-(4-acetylpiperazin-1-yl)-4-{[2-(4-fluorophenyl)-1,1-dimethylethyl]amino}-6-isopropyl-5,6-dihydro-7H-pyrrolo[3,4-d]pyrimidin-7-one

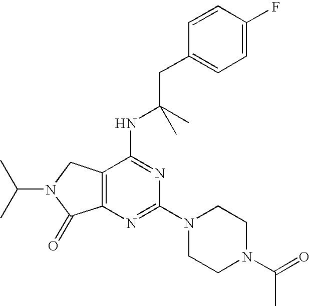

Following a procedure similar to that described in General Procedure 1 and after purification by silica gel chromatography (gradient 0-40% methanol: ethyl acetate) followed by preparative LCMS (gradient 45-65% CH₃CN in H₂O containing 0.1% trifluoroacetic acid), the title compound was obtained as its TFA salt. HPLC: k′ 6.91; Purity: >99% (215 nm), >99% (254 nm), >99% (280 nm); Rₜ: 1.82 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.25-1.29 (m, 6H), 1.48 (s, 6H), 2.14 (s, 3H), 3.32 (s, 2H), 3.69-3.78 (m, 4H), 3.85-3.99 (m, 4H), 4.19 (s, 2H), 4.42-4.51 (m, 1H), 6.91-6.99 (m, 2H), 7.04-7.11 (m, 2H). M.S. (calcd): 459.3 (MH⁺), M.S. (found): 459.3 (ESI) (MH⁺).

### Example 15

### 2-(4-acetylpiperazin-1-yl)-4-({[1-(4-chlorophenyl)cyclobutyl]methyl}amino)-6-isopropyl-5,6-dihydro-7H-pyrrolo[3,4-d]pyrimidin-7-one

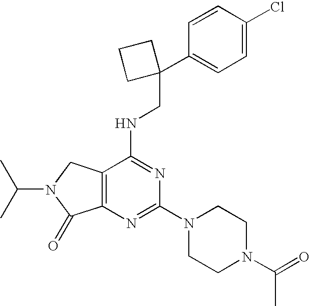

Following a procedure similar to that described in General Procedure 1 and after purification by silica gel chromatography (gradient 0-40% methanol:ethyl acetate) followed by preparative LCMS (gradient 45-65% CH₃CN in H₂O containing 0.1% trifluoroacetic acid), the title compound was obtained as its TFA salt. HPLC: k′ 7.65; Purity: >96% (215 nm), >96% (254 nm), >95% (280 nm); Rₜ: 1.99 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.84 Hz, 6H), 1.86-1.96 (m, 1H), 2.13-2.15 (m, 3H), 2.16-2.23 (m, 1H), 2.30-2.42 (m, 4H), 3.58-3.69 (m, 6H), 3.70-3.77 (m, 2H), 3.96 (s, 2H), 4.23 (s, 2H), 4.38-4.49 (m, 1H), 7.09-7.24 (m, 4H). M.S. (calcd): 497.2 (MH⁺), M.S. (found): 497.2 (ESI) (MH⁺).

### Example 16

### 2-(4-acetylpiperazin-1-yl)-4-{[2-(4-chlorophenyl)-2-methylpropyl]amino}-6-isopropyl-5,6-dihydro-7H-pyrrolo[3,4-d]pyrimidin-7-one

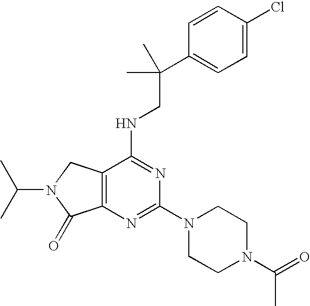

Following a procedure similar to that described in General Procedure 1 and after purification by silica gel chromatography (gradient 0-40% methanol:ethyl acetate) followed by preparative LCMS (gradient 45-65% CH₃CN in H₂O containing 0.1% trifluoroacetic acid), the title compound was obtained as its TFA salt. HPLC: k′ 7.43; Purity: >99% (215 nm), >99% (254 nm), >99% (280 nm); Rₜ: 1.94 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.84 Hz, 6H), 1.40 (s, 6H), 2.15 (s, 3H), 3.61-3.72 (m, 4H), 3.72-3.79 (m, 4H), 3.82 (dd, J=6.45, 4.10 Hz, 2H), 4.25 (s, 2H), 4.41-4.50 (m, 1H), 7.23-7.28 (m, 2H), 7.37-7.45 (m, 2H). M.S. (calcd): 485.2 (MH⁺), M.S. (found): 485.2 (ESI) (MH⁺).

### Example 17

### 4-{[bis(4-fluorophenyl)methyl]amino}-2-{[2-(dimethylamino)ethyl]amino}-6-isopropyl-5,6-dihydro-7H-pyrrolo[3,4-d]pyrimidin-7-one

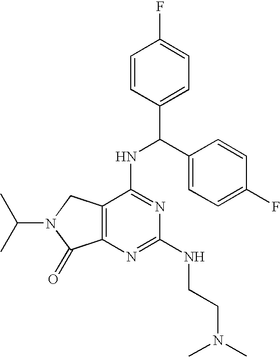

Following a procedure similar to that described in General Procedure 1 and after purification by reverse phase HPLC (gradient 45-65% CH₃CN in H₂O containing 10 mM NH₄HCO₃) and lyophilization from CH₃CN/H₂O, the title compound (45 mg, 26%) was obtained as a solid. HPLC: k′ 10.66; Purity: >96% (215 nm), >97% (254 nm), >97% (280 nm); Rₜ: 1.51 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=6.64 Hz, 6H), 2.80 (s, 6H), 3.11-3.15 (m, 1H), 3.30-3.35 (m, 1H), 3.64-3.69 (m, 2H), 4.33 (s, 2H), 4.47-4.51 (m, 1H), 6.58 (s, 1H), 7.10 (t, J=8.69 Hz, 4H), 7.31-7.34 (d, J=5.27 Hz, 4H). M.S. (calcd): 481.25 (MH⁺), M.S. (found): 481.2 (MH⁺).

### Example 18

### 4-{[(4-chlorophenyl)(phenyl)methyl]amino}-2-(4-ethylpiperazin-1-yl)-6-isopropyl-5,6-dihydro-7H-pyrrolo[3,4-d]pyrimidin-7-one

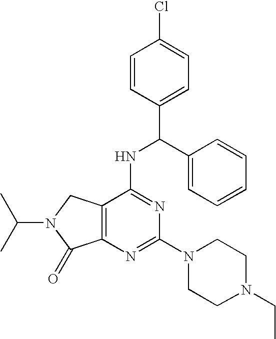

Following a procedure similar to that described in General Procedure 1 and after purification by reverse phase HPLC (gradient 55-75% CH₃CN in H₂O containing 10 mM NH₄HCO₃) and lyophilized from CH₃CN/H₂O, the residue was dissolved in CH₂Cl₂ and 3 drops of TFA was added. The mixture was stirred at rt for 2 h and concentrated under reduced pressure. After lyophilization from CH₃CN/H₂O, the title compound (35 mg, 20%) was obtained as a solid. HPLC: k′ 7.73; Purity: >93.3% (215 nm), >91% (254 nm), >90% (280 nm); Rₜ: 2.03 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28-1.34 (m, 9H), 2.76-2.82 (m, 2H), 3.10-3.17 (m, 4H), 3.45-3.49 (m, 2H), 4.27 (s, 2H), 4.51 (q, J=7.03 Hz, 1H), 4.80-4.85 (m. 2H), 6.42 (s, 1H), 7.27-7.36 (m, 9H). M.S. (calcd): 505.24 (MH⁺), M.S. (found): 505.3 (MH⁺). Found: C, 50.37; H, 4.67; N, 10.71. C₂₈H₃₃ClN₆O×2.4C₂HF₃O₂×0.2H₂O has C, 50.36; H, 4.61; N, 10.74%.

### Example 19

### 4-{[(4-chlorophenyl)(phenyl)methyl]amino}-2-[[2-(dimethylamino)ethyl](methyl)amino]-6-isopropyl-5,6-dihydro-7H-pyrrolo[3,4-d]pyrimidin-7-one

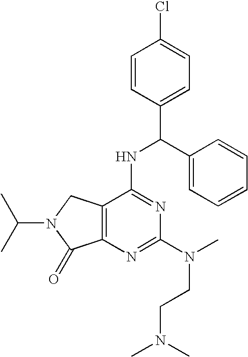

Following a procedure similar to that described in General Procedure 1 and after purification by reverse phase HPLC (gradient 55-75% CH₃CN in H₂O containing 10 mM NH₄HCO₃) and lyophilized from CH₃CN/H₂O, the residue was dissolved in CH₂Cl₂ and 3 drops of TFA was added. The mixture was stirred at rt for 2 h and concentrated under reduced pressure. After lyophilization from CH₃CN/H₂O, the title compound (35 mg, 20%) was obtained as a solid. HPLC: k′ 9.43; Purity: >99% (215 nm), >99% (254 nm), 94.0% (280 nm); Rₜ: 9.43 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.27 (d, J=6.84 Hz, 6H), 2.79 (s, 6H), 3.08 (s, 4H), 3.23 (t, J=5.08 Hz, 2H), 3.26 (dt, J=3.32, 1.66 Hz, 3H), 3.82 (d, J=5.08 Hz, 2H), 4.29 (s, 2H), 4.42-4.50 (m, 1H), 6.51 (s, 1H), 7.24-7.34 (m, 9H). M.S. (calcd): 493.248 (MH⁺), M.S. (found): 493.4 (MH⁺).

### Example 20

### 4-{[(4-chlorophenyl)(phenyl)methyl]amino}-6-isopropyl-2-(5-methyl-2,5-diazabicyclo[2.2.1]hept-2-yl)-5,6-dihydro-7H-pyrrolo[3,4-d]pyrimidin-7-one

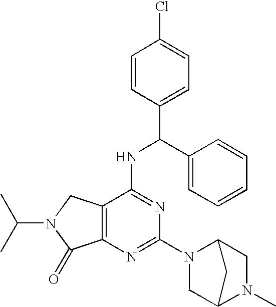

Following a procedure similar to that described in General Procedure 1 and after purification by reverse phase HPLC (gradient 55-75% CH₃CN in H₂O containing 10 mM NH₄HCO₃) and lyophilized from CH₃CN/H₂O, the residue was dissolved in CH₂Cl₂ and 3 drops of TFA was added. The mixture was stirred at rt for 2 h and concentrated under reduced pressure. After lyophilization from CH₃CN/H₂O, the title compound (65 mg, 37%) was obtained as a solid. HPLC: k′ 10.66; Purity: >99% (215 nm), >99% (254 nm), >99% (280 nm); Rₜ: 1.79 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.84 Hz, 6H), 2.20-2.35 (m, 2H), 2.89-2.93 (m, 3H), 3.72-3.78 (m, 2H), 4.32 (s, 2H), 4.37-4.41 (m, 1H), 4.45-4.53 (m, 1H), 4.94-4.98 (m, 1H), 6.50 (s, 1H), 7.27-7.38 (m, 9H). M.S. (calcd): 503.232 (MH⁺), M.S. (found): 503.3 (MH⁺). Found: C, 46.29; H, 3.97; N, 9.31. C₂₈H₃₁ClN₆O×3.4C₂HF₃O₂×0.7H₂O has C, 46.27; H, 3.99; N, 9.30%.

### Example 21

### 2-{[2-(dimethylamino)ethyl]amino}-4-[(9-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one

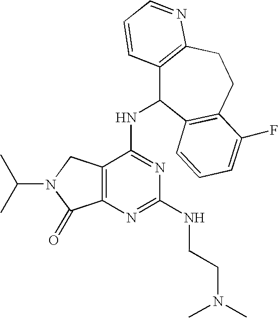

Following a procedure similar to that described in General Procedure 1 and starting from 9-fluoro-10,11-dihydro-5H-benzo[4,5]cyclohepta[1,2-b]pyridin-5-amine (for the preparation see: Cheng, Yun-Xing; Luo, Xuehong; Tomaszewski, Miroslaw; Walpole, Chistopher PCT Int. Appl. (2003), WO2003051276A2), the title compound was obtained as a solid (32 mg, 22%) following purification by reverse phase HPLC (gradient 45-65% CH₃CN in H₂O containing 0.1% trifluoroacetic acid) and lyophilization from CH₃CN/H₂O. HPLC: k′ 3.39; Purity: >94.7% (215 nm), >94% (254 nm), >93% (280 nm); Rₜ: 1.01 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.83 Hz, 6H), 2.90 (s, 6H), 3.25-3.32 (m, 3H), 3.41-3.58 (m, 1H), 3.63-3.68 (m, 2H), 3.78-3.88 (m, 1H), 4.31 (s, 2H), 4.49 (q, J=7.03 Hz, 1H), 6.75 (s, 1H), 7.07 (dt, J=0.98, 8.40 Hz, 1H), 7.28 (m, 1H), 7.36 (d, J=8.00 Hz, 1H), 7.75 (t, J=8.64 Hz, 1H), 8.58 (dd, J=1.37, 5.67 Hz, 1H), 8.67 (m, 1H). M.S. (calcd): 490.273 (MH⁺), M.S. (found): 490.3 (MH⁺).

### Example 22

### 2-{[2-(dimethylamino)ethyl]amino}-4-[(7-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one

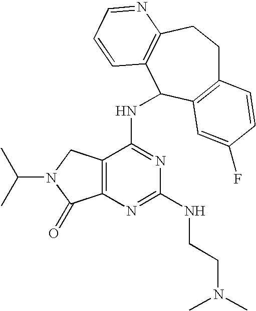

Following a procedure similar to that described in General Procedure 1 and starting from 7-fluoro-10,11-dihydro-5H-benzo[4,5]cyclohepta[1,2-b]pyridin-5-amine (for the preparation see: Cheng, Yun-Xing; Luo, Xuehong; Tomaszewski, Miroslaw; Walpole, Chistopher PCT Int. Appl. (2003), WO2003051276A2), the title compound was obtained as a solid (45 mg, 30%) following purification by reverse phase HPLC (gradient 45-65% CH₃CN in H₂O containing 0.1% trifluoroacetic acid) and lyophilization from CH₃CN/H₂O. HPLC: k′ 3.38; Purity: >96% (215 nm), >95% (254 nm), >93% (280 nm); Rₜ: 1.01 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.8 Hz, 6H), 2.89 (s, 6H), 2.94-2.96 (m, 1H), 3.31-3.39 (m, 2H), 3.42-3.50 (m, 1H), 3.53-3.78 (m, 4H), 4.37 (s, 2H), 4.49 (q, J=7.03 Hz, 1H), 6.87 (s, 1H), 7.05 (dt, J=2.35, 9.18 Hz, 1H), 7.32-7.38 (m, 2H), 7.83 (t, J=5.86 Hz, 1H), 8.64 (d, J=5.67 Hz, 1H), 8.72 (dd, J=2.15, 6.84 Hz, 1H). M.S. (calcd): 490.273 (MH⁺), M.S. (found): 490.3 (MH⁺).

### Example 23

### 4-{[bis(4-fluorophenyl)methyl]amino}-6-isopropyl-2-(5-methyl-2,5-diazabicyclo[2.2.1]hept-2-yl)-5,6-dihydro-7H-pyrrolo[3,4-d]pyrimidin-7-one

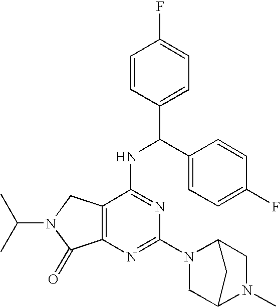

Following a procedure similar to that described in General Procedure 1 and after purification by reverse phase HPLC (gradient 35-55% CH₃CN in H₂O containing 0.1% trifluoroacetic acid), The title compound was obtained as a solid (46 mg, 30%) following lyophilization from CH₃CN/H₂O. HPLC: k′ 6.43; Purity: >95% (215 nm), >94% (254 nm), >96% (280 nm); Rₜ: 1.71 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.64 Hz, 6H), 1.34-1.44 (m, 2H), 2.20-2.36 (m, 2H), 2.85-2.98 (m, 3H), 3.70-3.81 (m, 2H), 4.32 (s, 2H), 4.38-4.40 (m, 1H), 4.49 (dt, J=13.38, 6.79 Hz, 1H), 4.98-5.02 (m, 1H), 6.52 (s, 1H), 7.09 (t, J=8.59 Hz, 4H), 7.32-7.43 (m, 4H). M.S. (calcd): 505.252 (MH⁺), M.S. (found): 505.3 (MH⁺).

### Example 24

### 4-{[bis(4-fluorophenyl)methyl]amino}-2-[[2-(dimethylamino)ethyl](methyl)amino]-6-isopropyl-5,6-dihydro-7H-pyrrolo[3,4-d]pyrimidin-7-one

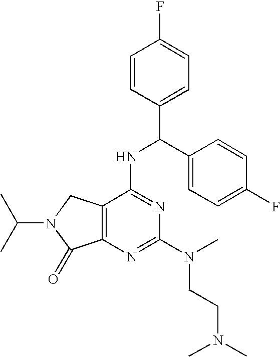

Following a procedure similar to that described in General Procedure 1 and after purification by reverse phase HPLC (gradient 35-55% CH₃CN in H₂O containing 0.1% trifluoroacetic acid), The title compound was obtained as a solid (45 mg, 30%) following lyophilization from CH₃CN/H₂O. HPLC: k′ 7.70; Purity: >95% (215 nm), >99% (254 nm), >99% (280 nm); Rₜ: 2.00 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=6.84 Hz, 6H), 2.88 (s, 6H), 3.06-3.13 (m, 5H), 3.82 (t, J=5.28 Hz, 2H), 4.30 (s, 2H), 4.47-4.51 (m, 1H), 6.55 (s, 1H), 7.05-7.10 (m, 4H), 7.32-7.35 (m, 4H). M.S. (calcd): 495.268 (MH⁺), M.S. (found): 495.2 (MH⁺).

### Example 25

### 2-(4-acetylpiperazin-1-yl)-6-isopropyl-4-({1-[4-(trifluoromethoxy)phenyl]ethyl}amino)-5,6-dihydro-7H-pyrrolo[3,4-d]pyrimidin-7-one

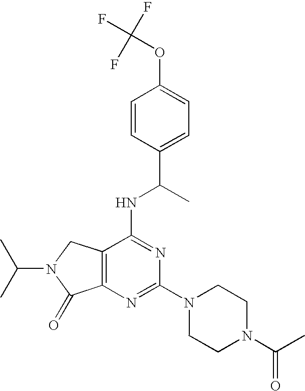

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (35 mg, 34%) was obtained as a solid. HPLC: k′ 13.73; Purity: >99% (215 nm), >99% (254 nm), >99% (280 nm); Rₜ: 1.92 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (s, 3H), 1.31 (s, 3H), 1.55 (d, J=7.0 Hz, 3H), 2.09 (s, 3H), 3.42 (m, 4H), 3.70 (m, 4H), 4.26 (s, 2H), 4.52 (hep, J=6.8 Hz, 1H), 5.24 (q, J=7.0 Hz, 1H), 7.20 (d, J=8.2 Hz, 2H), 7.46 (d, J=8.2 Hz, 2H). M.S. (calcd): 507.5 (MH⁺), M.S. (found): 507.3 (ESI) (MH⁺)

### Example 26

### 2-(4-acetylpiperazin-1-yl)-4-{[1-(4-ethoxyphenyl)ethyl]amino}-6-isopropyl-5,6-dihydro-7H-pyrrolo[3,4-d]pyrimidin-7-one

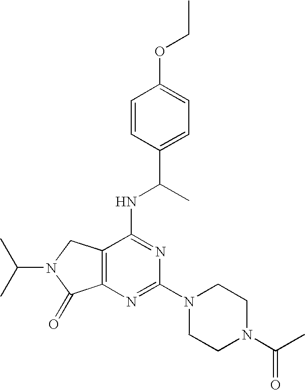

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (22 mg, 23%) was obtained as a solid. HPLC: k′ 6.30; Purity: >96% (215 nm), >98% (254 nm), >98% (280 nm); Rₜ: 1.68 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.8 Hz, 6H), 1.34 (t, J=7.0 Hz, 3H), 1.53 (d, J=7.0 Hz, 3H), 2.11 (s, 3H), 3.60-3.35 (m, 4H), 3.87-3.64 (m, 4H), 3.98 (m, 2H), 4.22 (s, 2H), 4.51 (m, 1H), 5.21 (m, 1H), 6.83 (d, J=8.4 Hz, 2H), 7.27 (d, J=8.4 Hz, 2H). M.S. (calcd): 467.6 (MH⁺), M.S. (found): 467.3 (ESI) (MH⁺).

### Example 27

### 2-(4-acetylpiperazin-1-yl)-4-{[(4-chlorophenyl)(cyclopropyl)methyl]amino}-6-isopropyl-5,6-dihydro-7H-pyrrolo[3,4-d]pyrimidin-7-one

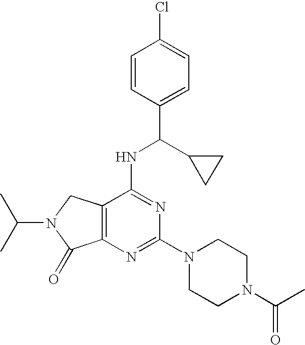

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (21 mg, 21%) was obtained as a solid. HPLC: k′ 13.15; Purity: >95% (215 nm), >95% (254 nm), >97% (280 nm); Rₜ: 1.84 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 0.44 (m, 2H), 0.64 (m, 2H), 1.26 (m, 1H), 1.32 (d, J=6.6 Hz, 6H), 2.11 (s, 3H), 3.30-3.75 (m, 8H), 4.28 (s, 2H), 4.38 (d, J=9.4 Hz, 1H), 4.52 (hept, J=6.6 Hz, 1H), 7.30 (d, J=8.4 Hz, 2H), 7.38 (d, J=8.4 Hz, 2H). M.S. (calcd): 484.0 (MH⁺), M.S. (found): 484.3 (ESI) (MH⁺).

### Example 28

### 2-(4-acetylpiperazin-1-yl)-6-isopropyl-4-({1-[4-(trifluoromethyl)phenyl]ethyl}amino)-5,6-dihydro-7H-pyrrolo[3,4-d]pyrimidin-7-one

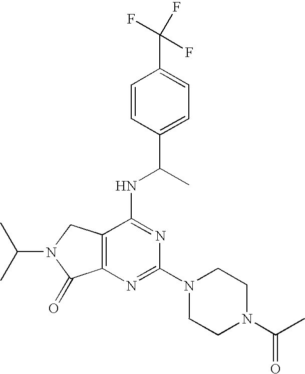

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (29 mg, 28%) was obtained as a solid. HPLC: k′=13.27; Purity: >99% (215 nm), >99% (254 nm), >99% (280 nm); Rₜ: 1.86 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.32 (d, J=6.6 Hz, 6H), 1.57 (d, J=7.2 Hz, 3H), 2.09 (s, 3H), 3.70-3.30 (m, 8H), 4.28 (m, 2H), 4.52 (hept, J=6.6 Hz, 1H), 5.27 (m, 1H), 7.51 (d, J=8.2 Hz, 2H), 7.61 (d, J=8.2 Hz, 2H). M.S. (calcd): 491.5 (MH⁺), M.S. (found): 491.3 (ESI) (MH⁺).

### Example 29

### 2-(4-acetylpiperazin-1-yl)-6-isopropyl-4-{[1-(4-propylphenyl)ethyl]amino}-5,6-dihydro-7H-pyrrolo[3,4-d]pyrimidin-7-one

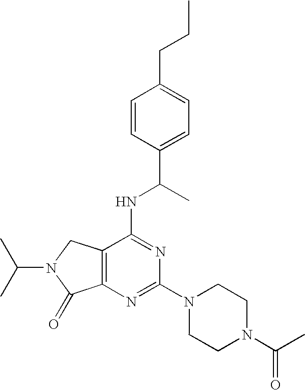

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (25 mg, 27%) was obtained as a solid. HPLC: k′ 14.38; Purity: >92% (215 nm), >98% (254 nm), >98% (280 nm); Rₜ: 2.00 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.02 (t, J=7.4 Hz, 3H), 1.33 (m, 6H), 1.96 (m, 2H), 2.12 (s, 3H), 3.63-3.42 (m, 4H), 3.82-3.65 (m, 6H), 4.35 (s, 2H), 4.51 (m, 2H), 4.92 (s, 2H), 5.12 (m, 1H), 7.57 (d, J=8.2 Hz, 2H), 7.63 (d, J=8.2 Hz, 2H). M.S. (calcd): 465.6 (MH⁺), M.S. (found): 465.3 (ESI) (MH⁺)

### Example 30

### 2-(4-acetylpiperazin-1-yl)-6-isopropyl-4-{[4-(trifluoromethoxy)benzyl]amino}-5,6-dihydro-7H-pyrrolo[3,4-d]pyrimidin-7-one

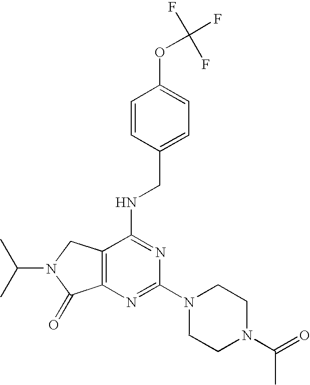

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (27 mg, 27%) was obtained as a solid. HPLC: k′ 12.86; Purity: >99% (215 nm), >99% (254 nm), >99% (280 nm); Rₜ: 1.80 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.8 Hz, 6H), 2.11 (s, 3H), 3.48 (m, 2H), 3.53 (m, 2H), 3.75 (m, 2H), 3.82 (m, 2H), 4.22 (s, 2H), 4.52 (m, 1H), 4.69 (s, 2H), 7.21 (d, J=8.0 Hz, 2H), 7.45 (d, J=8.0 Hz, 2H). M.S. (calcd): 493.5 (MH⁺), M.S. (found): 493.3 (ESI) (MH⁺).

### Example 31

### 5-[2-(4-{4-[(4-chlorophenyl)amino]-6-isopropyl-7-oxo-6,7-dihydro-5H-pyrrolo[3,4-d]pyrimidin-2-yl}piperazin-1-yl)-2-oxoethyl]imidazolidine-2,4-dione

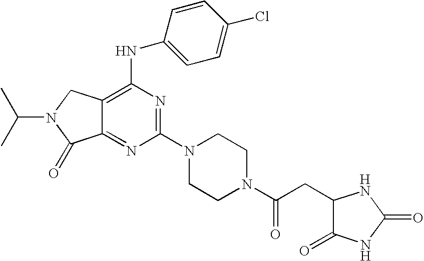

The titled compound was reported in the literature (Aharony D. et al., The Journal of Pharmacology and Experimental Therapeutics, 1995, 274, 1216-1221). By following the literature method, the final compound was purified by preparative LCMS (gradient 10-40% CH₃CN in H₂O pH=10, buffering with NH₄HCO₃/NH₄OH) to give the title compound. This material was lyophilized from CH₃CN/H₂O to produce a solid (125 mg). HPLC: k′ 3.22; Purity: >99% (215 nm), >99% (254 nm), >99% (280 nm); Rₜ: 1.80 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.84 Hz, 6H), 2.65 (dd, J=16.41, 9.37 Hz, 1H), 2.99 (dd, J=16.50, 3.03 Hz, 1H), 3.53-3.59 (m, 2H), 3.60-3.65 (m, 3H), 3.78-3.83 (m, 2H), 3.83-3.88 (m, 2H), 4.26 (dd, J=9.37, 2.93 Hz, 1H), 4.44-4.59 (m, 2H), 7.30 (d, J=8.79 Hz, 2H), 7.66 (d, J=8.79 Hz, 2H). M.S. (calcd): 528.0 (MH⁺), M.S. (found): 528.2 (ESI) (MH⁺).

### Example 32

### 2-(4-acetylpiperazin-1-yl)-6-isopropyl-4-({1-[4-(trifluoromethyl)phenyl]propyl}amino)-5,6-dihydro-7H-pyrrolo[3,4-d]pyrimidin-7-one

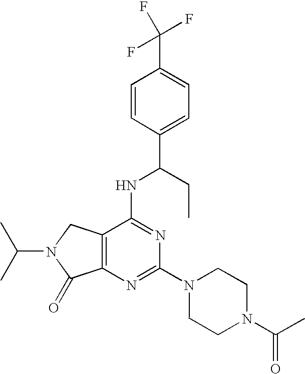

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (27 mg, 26%) was obtained as a solid. HPLC: k′ 14.29; Purity: >93% (215 nm), >99% (254 nm), >99% (280 nm); Rₜ: 1.99 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm. 0.90 (t, J=7.2 Hz, 3H), 1.31 (d, J=6.6 Hz, 6H), 1.60 (m, 2H), 2.10 (s, 3H), 2.54 (dd, J=7.8, 7.4 Hz, 2H), 3.35-3.65 (m, 4H), 3.69 (m, 2H), 3.70 (m, 1H), 3.74 (m, 1H), 3.80 (m, 1H), 4.52 (m, 1H), 7.11 (d, J=8.1 Hz, 2H), 7.26 (d, J=8.1 Hz, 2H). M.S. (calcd): 505.6 (MH⁺), M.S. (found): 505.3 (ESI) (MH⁺).

### Example 33

### 2-(4-acetylpiperazin-1-yl)-4-{[trans-2-(4-chlorophenyl)cyclopentyl]amino}-6-isopropyl-5,6-dihydro-7H-pyrrolo[3,4-d]pyrimidin-7-one

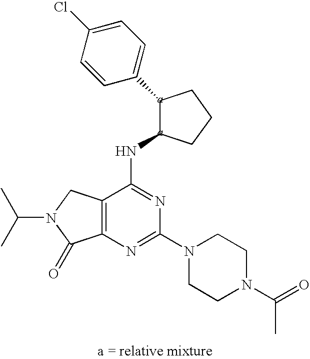

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (365 mg, 66%) was obtained as a solid. HPLC: k′ 15.62; Purity: >99% (215 nm), >99% (254 nm), >95% (280 nm); Rₜ: 2.16 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm. 1.26 (d, J=6.7 Hz, 6H), 1.80 (m, 2H), 1.95 (m, 2H), 2.15 (s, 3H), 2.22 (m, 1H), 2.32 (m, 1H), 3.05 (m, 1H), 3.75-3.50 (m, 8H), 4.22 (s, 2H), 4.49 (m, 1H), 4.55 (m, 1H), 7.26 (m, 4H). M.S. (calcd): 498.0 (MH⁺), M.S. (found): 498.0 (ESI) (MH⁺).

### Example 34

### 2-(4-acetylpiperazin-1-yl)-6-isopropyl-4-{[(1R)-1-(4-methylphenyl)ethyl]amino}-5,6-dihydro-7H-pyrrolo[3,4-d]pyrimidin-7-one

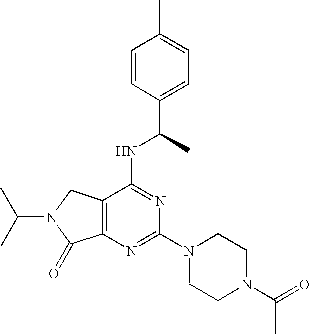

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (24 mg, 28%) was obtained as a solid. HPLC: k′ 11.81; Purity: >99% (215 nm), >99% (254 nm), >99% (280 nm); Rₜ: 1.67 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm. 1.31 (d, J=6.6 Hz, 6H), 1.53 (d, J=7.0 Hz, 3H), 2.10 (s, 3H), 2.28 (s, 3H), 3.45 (m, 4H), 3.73 (m, 4H), 4.23 (s, 2H), 4.52 (hept, J=6.6 Hz, 1H), 5.20 (m, 1H), 7.10 (d, J=7.9 Hz, 2H), 7.23 (d, J=7.9 Hz, 2H). M.S. (calcd): 437.6 (MH⁺), M.S. (found): 437.3 (ESI) (MH⁺).

### Example 35

### {[2-(4-acetylpiperazin-1-yl)-6-isopropyl-7-oxo-6,7-dihydro-5H-pyrrolo[3,4-d]pyrimidin-4-yl]amino}[4-(trifluoromethyl)phenyl]acetic acid

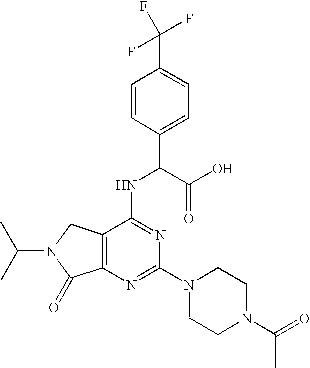

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (27 mg, 25%) was obtained as a solid. HPLC: k′ 12.28; Purity: >99% (215 nm), >99% (254 nm), >99% (280 nm); Rₜ: 1.73 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm. 1.31 (d, J=6.6 Hz, 6H), 2.11 (s, 3H), 3.46 (m, 2H), 3.52 (m, 2H), 3.73 (m, 2H), 3.80 (m, 2H), 4.24 (s, 2H), 4.53 (hept, J=6.6 Hz, 1H), 4.75 (s, 2H), 7.54 (d, J=8.0 Hz, 2H), 7.61 (d, J=8.0 Hz, 2H). M.S. (calcd): 521.5 (MH⁺), M.S. (found): 521.5 (ESI) (MH⁺).

### Example 36

### 2-(4-acetylpiperazin-1-yl)-6-isopropyl-4-{[1-(4-methylphenyl)propyl]amino}-5,6-dihydro-7H-pyrrolo[3,4-d]pyrimidin-7-one

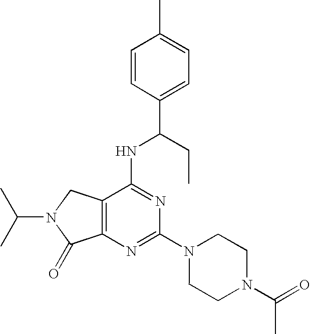

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (24 mg, 25%) was obtained as a solid. HPLC: k′ 13.02; Purity: >99% (215 nm), >99% (254 nm), >99% (280 nm); Rₜ: 1.73 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm. 0.96 (t, J=7.2 Hz, 3H), 1.31 (m, 6H), 1.82 (m, 1H), 1.91 (m, 1H), 2.12 (s, 3H), 2.28 (s, 3H), 3.45 (m, 3H), 3.54 (m, 1H), 3.72 (m, 3H), 3.78 (m, 1H), 4.24 (s, 2H), 4.51 (sep, J=6.6 Hz, 1H), 4.99 (m, 1H), 7.10 (d, J=8.0 Hz, 2H), 7.25 (d, J=8.0 Hz, 2H). M.S. (calcd): 451.6 (MH⁺), M.S. (found): 451.2 (ESI) (MH⁺).

### Example 37

### 4-[(4-benzylphenyl)amino]-6-isopropyl-2-morpholin-4-yl-5,6-dihydro-7H-pyrrolo[3,4-d]pyrimidin-7-one

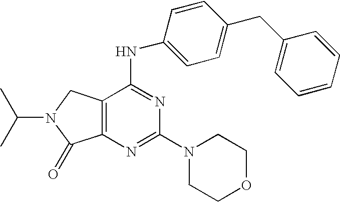

By following the literature method (Reference: D. AHARONY, C. K. BUCKNER, J. L. ELLIS, S. V. GHANEKAR, A. GRAHAM, J. S. KAYS, J. LITTLE, S. MEEKER, S. C. MILLER, B. Y J. UNDEM and I. WALDRON The Journal of Pharmacology and Experimental Therapeutics, 1995, 274, 1216-1221, which incorporated by reference herein for its disclosure in making Compound 37), the final compound was purified by preparative LCMS (gradient 10-40% CH₃CN in H₂O pH=10, buffering with NH₄HCO₃/NH₄OH) to give the title compound. This material was lyophilized from CH₃CN/H₂O to produce a solid. (125 mg). HPLC: k′ 5.08; Purity: >95% (215 nm), >99% (254 nm), >99% (280 nm); Rₜ: 1.73 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.20 (d, J=6.8 Hz, 6H), 3.71 (m, 4H), 3.77 (m, 4H), 3.99 (s, 2H), 4.13 (m, 2H), 4.48 (m, 1H), 7.19 (m, 5H), 7.29 (m, 2H), 7.48 (d, J=8.6 Hz, 2H). M.S. (calcd): 444.6 (MH⁺), M.S. (found): 444.6 (ESI) (MH⁺).

### Example 38

### 2-(4-acetylpiperazin-1-yl)-6-isopropyl-4-{[(1-methyl-1,2,3,4-tetrahydroquinolin-6-yl)methyl]amino}-5,6-dihydro-7H-pyrrolo[3,4-d]pyrimidin-7-one

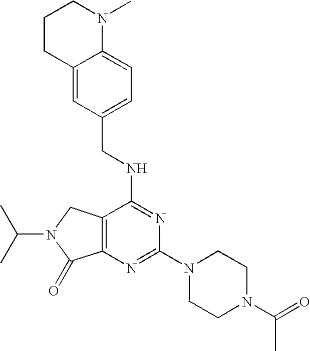

Following a procedure similar to that described in General Procedure 1 and after purification by preparative LCMS (gradient 45-65% CH₃CN in H₂O pH=10, buffering with NH₄HCO₃/NH₄OH) and lyophilization from CH₃CN/H₂O, the title compound (24 mg, 25%) was obtained as a solid. HPLC: k′ 8.23; Purity: >99% (215 nm), >99% (254 nm), >99% (280 nm); Rₜ: 1.20 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃Cl) δ ppm. 1.28 (d, J=6.6 Hz, 6H), 1.93 (m, 2H), 2.12 (s, 3H), 2.69 (t, J=6.4 Hz, 2H), 2.81 (s, 3H), 3.14 (t, J=5.7 Hz, 2H), 3.55 (m, 4H), 3.82 (m, 4H), 4.17 (s, 2H), 4.49 (s, 2H), 4.50 (hep, J=6.6 Hz, 1H), 6.55 (d, J=8.4 Hz, 1H), 6.91 (s, 1H), 7.00 (m, 1H). M.S. (calcd): 478.6 (MH⁺), M.S. (found): 478.3 (ESI) (MH⁺).

### Example 39

### 2-(4-acetylpiperazin-1-yl)-4-[2-ethyl-2-(4-methylphenyl)hydrazino]-6-isopropyl-5,6-dihydro-7H-pyrrolo[3,4-d]pyrimidin-7-one

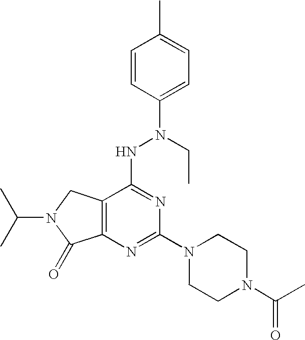

Following a procedure similar to that described in General Procedure 1 and after purification by preparative LCMS (gradient 10-40% CH₃CN in H₂O pH=10, buffering with NH₄HCO₃/NH₄OH) and lyophilization from CH₃CN/H₂O, the title compound (23 mg, 25%) was obtained as a solid. HPLC: k′ 13.99; Purity: >99% (215 nm), >99% (254 nm), >99% (280 nm); Rₜ: 1.95 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm. 1.12 (d, J=6.5 Hz, 3H), 1.20 (t, J=7.2 Hz, 3H), 1.25 (d, J=6.5 Hz, 3H), 2.15 (s, 3H), 2.24 (s, 3H), 3.62 (m, 5H), 3.92 (m, 5H), 4.40 (m, 2H), 4.60 (s, 1H), 6.88 (d, J=8.3 Hz, 2H), 7.09 (d, J=8.3 Hz, 2H). M.S. (calcd): 452.6 (MH⁺), M.S. (found): 452.3 (ESI) (MH⁺)

### Example 40

### 2-(4-acetylpiperazin-1-yl)-6-isopropyl-4-{[1-(4-isopropylphenyl)ethyl]amino}-5,6-dihydro-7H-pyrrolo[3,4-d]pyrimidin-7-one

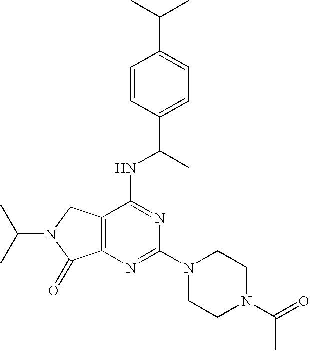

Following a procedure similar to that described in General Procedure 1 and after purification by preparative LCMS (gradient 35-55% CH₃CN in H₂O pH=10, buffering with NH₄HCO₃/NH₄OH) and lyophilization from CH₃CN/H₂O, the title compound (88 mg, 75%) was obtained as a solid. HPLC: k′ 14.18; Purity: >96% (215 nm), >99% (254 nm), >99% (280 nm); Rₜ: 1.97 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.21 (d, J=7.03 Hz, 6H), 1.32 (dd, J=6.64, 1.95 Hz, 6H), 1.61 (d, J=7.03 Hz, 3H), 2.12-2.14 (m, 3H), 2.65 (s, 2H), 2.83-2.90 (m, 1H), 3.60 (d, J=7.03 Hz, 2H), 3.62-3.71 (m, 2H), 3.71-3.78 (m, 2H), 3.80-3.86 (m, 2H), 4.37 (s, 1H), 4.47 (s, 1H), 7.21 (d, J=8.20 Hz, 2H), 7.29-7.32 (m, 2H). M.S. (calcd): 465.3 (MH⁺), M.S. (found): 465.3 (ESI) (MH⁺).

### Example 41

### 2-(4-acetylpiperazin-1-yl)-4-{[2-(4-chlorophenyl)ethyl]amino}-6-isopropyl-5,6-dihydro-7H-pyrrolo[3,4-d]pyrimidin-7-one

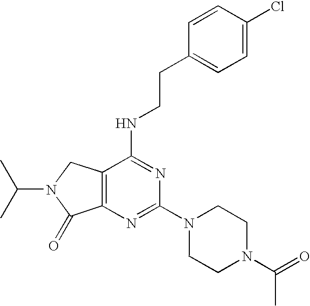

Following a procedure similar to that described in General Procedure 1 and after purification by silica gel chromatography (gradient 0-50% methanol: ethyl acetate) followed by reverse phase HPLC (gradient 35-55% CH₃CN in H₂O containing 0.1% trifluoroacetic acid) and lyophilization from CH₃CN/H₂O, the title compound (40 mg, 34%) was obtained as its TFA salt. HPLC: k′ 11.9; Purity: >89% (215 nm), >91% (254 nm), >93% (280 nm); Rₜ: 1.68 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29-1.35 (m, 6H), 2.14-2.17 (s, 3H), 2.96 (t, J=6.93 Hz, 2H), 3.69-3.76 (m, 4H), 3.76-3.84 (m, 4H), 3.88-3.90 (m, 2H), 3.97 (s, 1H), 4.26 (s, 2H), 7.21-7.30 (m, 4H). M.S. (calcd): 457.2 (MH⁺), M.S. (found): 457.3 (ESI) (MH⁺).

### Example 42

### 2-(4-acetylpiperazin-1-yl)-6-isopropyl-4-{[2-(4-methylphenyl)ethyl]amino}-5,6-dihydro-7H-pyrrolo[3,4-d]pyrimidin-7-one

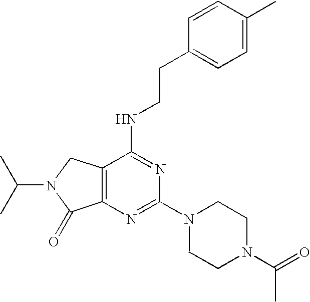

Following a procedure similar to that described in General Procedure 1 and after purification by reverse phase HPLC (gradient 35-55% CH₃CN in H₂O containing 0.1% trifluoroacetic acid) and lyophilization from CH₃CN/H₂O, the title compound (65 mg, 58%) was obtained as its TFA salt. HPLC: k′ 11.7; Purity: >92% (215 nm), >99% (254 nm), >99% (280 nm); Rₜ: 1.65 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=7.03 Hz, 6H), 2.16 (s, 3H), 2.28 (s, 3H), 2.92 (t, J=7.23 Hz, 1H), 3.65-3.73 (m, 4H), 3.77 (t, J=7.42 Hz, 4H), 3.81-3.85 (m, 2H), 3.89 (d, J=6.25 Hz, 2H), 4.24 (s, 2H), 7.10 (d, J=1.56 Hz, 4H). M.S. (calcd): 437.3 (MH⁺), M.S. (found): 437.3 (ESI) (MH⁺).

### Example 43

### 2-(4-acetylpiperazin-1-yl)-6-isopropyl-4-[(4-isopropylbenzyl)amino]-5,6-dihydro-7H-pyrrolo[3,4-d]pyrimidin-7-one

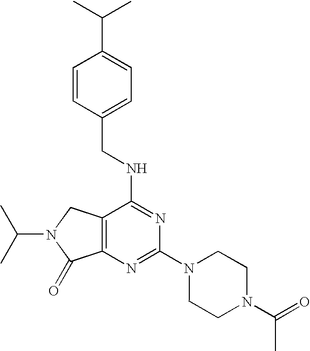

Following a procedure similar to that described in General Procedure 1 and after purification by silica gel chromatography (gradient 0-50% methanol: ethyl acetate) followed by reverse phase HPLC (gradient 35-55% CH₃CN in H₂O containing 0.1% trifluoroacetic acid) and lyophilization from CH₃CN/H₂O, the title compound (60 mg, 52%) was obtained as its TFA salt. HPLC: k′ 13.5; Purity: >99% (215 nm), >99% (254 nm), >99% (280 nm); Rₜ: 1.89 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.21 (d, J=7.03 Hz, 6H), 1.31 (d, J=6.84 Hz, 6H), 2.14 (s, 3H), 2.84-2.91 (m, 1H), 3.64-3.71 (m, 4H), 3.81-3.84 (m, 2H), 3.88-3.91 (m, 2H), 4.32 (s, 2H), 4.44-4.51 (m, 1H), 4.72 (s, 2H), 7.19-7.23 (m, 2H), 7.28-7.32 (m, 2H). M.S. (calcd): 451.3 (MH⁺), M.S. (found): 451.2 (ESI) (MH⁺).

### Example 44

### 4-[2-(4-chlorobenzyl)pyrrolidin-1-yl]-6-isopropyl-2-[(2-methoxyethyl)amino]-5,6-dihydro-7H-pyrrolo[3,4-d]pyrimidin-7-one

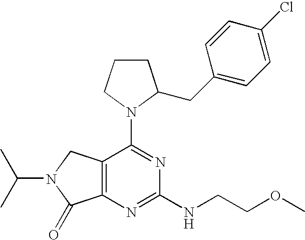

Following a procedure similar to that described in General Procedure 1 and after purification by reverse phase HPLC (gradient 45-65% CH₃CN in H₂O containing 0.1% trifluoroacetic acid) and lyophilization from CH₃CN/H₂O, the title compound (20 mg, 18%) was obtained as its TFA salt. HPLC: k′ 15.0; Purity: >99% (215 nm), >99% (254 nm), >99% (280 nm); Rₜ: 2.08 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31-1.36 (m, 6H), 1.89 (s, 2H), 2.08 (s, 2H), 2.74 (s, 2H), 3.25 (s, 2H), 3.36 (s, 3H), 3.60 (t, J=5.47 Hz, 2H), 3.69 (d, J=4.69 Hz, 2H), 3.85 (s, 1H), 3.94 (s, 1H), 4.71 (s, 2H), 7.22-7.33 (m, 4H). M.S. (calcd): 444.2 (MH⁺), M.S. (found): 444.3 (ESI) (MH⁺).

### Example 45

### N-[2-({4-[2-(4-chlorobenzyl)pyrrolidin-1-yl]-6-isopropyl-7-oxo-6,7-dihydro-5H-pyrrolo[3,4-d]pyrimidin-2-yl}amino)ethyl]acetamide

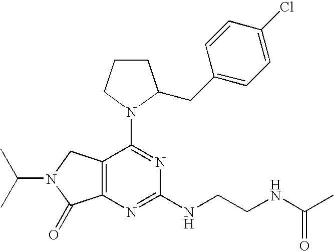

Following a procedure similar to that described in General Procedure 1 and after purification by reverse phase HPLC (gradient 45-65% CH₃CN in H₂O containing 10 mM NH₄HCO₃) and lyophilization from CH₃CN/H₂O, the resulting solid was dissolved in a 50% CH₃CN in H₂O containing 0.1% trifluoroacetic acid and concentrated under reduced pressure, the residue was lyophilized from CH₃CN/H₂O to provide the title compound (25 mg, 21%) as its TFA salt. HPLC: k′ 13.0; Purity: >99% (215 nm), >99% (254 nm), >99% (280 nm); Rₜ: 1.81 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.34 (dd, J=6.64, 4.49 Hz, 6H), 1.84-1.97 (m, 5H), 2.00-2.15 (m, 2H), 2.72-2.84 (m, 2H), 3.22-3.28 (m, 2H), 3.41-3.48 (m, 2H), 3.52-3.59 (m, 1H), 3.69 (s, 1H), 3.89-3.99 (m, 2H), 4.37-4.54 (m, 2H), 7.16-7.38 (m, 4H). M.S. (calcd): 471.2 (MH⁺), M.S. (found): 471.3 (ESI) (MH⁺).

### Example 46

### 2-(4-acetylpiperazin-1-yl)-4-[2-(2-chlorobenzyl)pyrrolidin-1-yl]-6-isopropyl-5,6-dihydro-7H-pyrrolo[3,4-d]pyrimidin-7-one

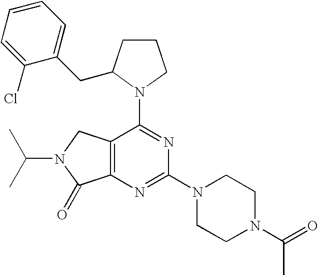

Following a procedure similar to that described in General Procedure I and after purification by reverse phase HPLC (gradient 35-55% CH₃CN in H₂O containing 0.1% trifluoroacetic acid) and lyophilization from CH₃CN/H₂O, the title compound (72 mg, 58%) was obtained as its TFA salt. HPLC: k′ 13.8; Purity: >99% (215 nm), >99% (254 nm), >99% (280 nm); Rₜ: 1.93 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.16-1.50 (m, 6H), 1.91 (s, 2H), 2.16 (m, 7H), 3.31-3.44 (m, 2H), 3.64-3.93 (m, 8H), 3.94-4.20 (m, 1H), 4.39-4.62 (m, 1H), 4.66-4.81 (m, 2H), 7.11-7.50 (m, 4H). M.S. (calcd): 497.2 (MH⁺), M.S. (found): 497.2 (ESI) (MH⁺).

### Example 47

### 2-(4-acetylpiperazin-1-yl)-6-isopropyl-4-[2-(4-methylbenzyl)pyrrolidin-1-yl]-5,6-dihydro-7H-pyrrolo[3,4-d]pyrimidin-7-one

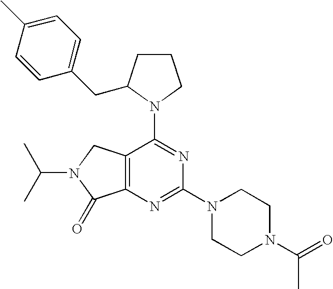

Following a procedure similar to that described in General Procedure 1 and after purification by reverse phase HPLC (gradient 35-55% CH₃CN in H₂O containing 0.1% trifluoroacetic acid) and lyophilization from CH₃CN/H₂O, the title compound (65 mg, 54%) was obtained as its TFA salt. HPLC: k′ 14.1; Purity: >92% (215 nm), >94% (254 nm), >99% (280 nm); Rₜ: 1.96 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.32 (s, 6H), 1.90 (s, 2H), 2.08 (s, 2H), 2.16 (s, 3H), 2.29 (s, 3H), 2.72 (s, 2H), 3.19 (s, 2H), 3.74 (s, 4H), 3.90 (s, 5H), 4.48 (s, 1H), 4.76 (s, 2H), 7.10 (s, 4H). M.S. (calcd): 477.3 (MH⁺), M.S. (found): 477.2 (ESI) (MH⁺).

### Example 48

### 2-(4-acetylpiperazin-1-yl)-4-[2-(3-chlorobenzyl)pyrrolidin-1-yl]-6-isopropyl-5,6-dihydro-7H-pyrrolo[3,4-d]pyrimidin-7-one

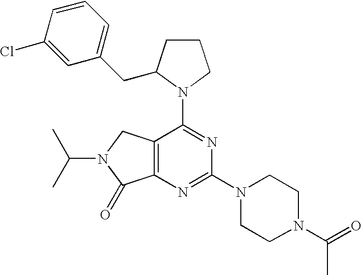

Following a procedure similar to that described in General Procedure 1 and after purification by reverse phase HPLC (gradient 35-55% CH₃CN in H₂O containing 0.1% trifluoroacetic acid) and lyophilization from CH₃CN/H₂O, the title compound (85 mg, 69%) was obtained as its TFA salt. HPLC: k′ 14.0; Purity: >95% (215 nm), >95% (254 nm), >99% (280 nm); Rₜ: 1.95 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.35 (s, 6H), 1.92 (s, 2H), 2.08-2.19 (m, 5H), 2.75-2.86 (m, 1H), 3.34 (s, 1H), 3.70-3.80 (m, 5H), 3.83-3.93 (m, 5H), 3.96 (d, J=8.01 Hz, 1H), 4.44-4.55 (m, 1H), 4.74 (s, 2H), 7.18 (d, J=7.23 Hz, 1H), 7.22-7.32 (m, 3H). M.S. (calcd): 497.2 (MH⁺), M.S. (found): 497.2 (ESI) (MH⁺).

### Example 49

### 4-[2-(4-chlorobenzyl)pyrrolidin-1-yl]-6-isopropyl-2-(3-oxopiperazin-1-yl)-5,6-dihydro-7H-pyrrolo[3,4-d]pyrimidin-7-one

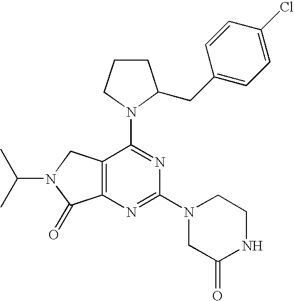

Following a procedure similar to that described in General Procedure 1 and after purification by reverse phase HPLC (gradient 45-65% CH₃CN in H₂O containing 10 mM NH₄HCO₃) and lyophilization from CH₃CN/H₂O, the resulting solid was dissolved in a 50% CH₃CN in H₂O containing 0.1% trifluoroacetic acid and concentrated under reduced pressure, the residue was lyophilized from CH₃CN/H₂O to provide the title compound (25 mg, 21%) as its TFA salt. HPLC: k′ 13.5; Purity: >99% (215 nm), >99% (254 nm), >99% (280 nm); Rₜ: 1.89 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.27-1.36 (m, 6H), 1.88 (s, 2H), 1.99-2.07 (m, 2H), 2.76 (s, 1H), 3.20 (d, J=7.42 Hz, 1H), 3.44 (t, J=5.27 Hz, 2H), 3.77 (d, J=9.77 Hz, 1H), 3.85 (s, 1H), 4.03 (td, J=5.27, 2.93 Hz, 2H), 4.39 (s, 2H), 4.50 (d, J=6.64 Hz, 1H), 4.67 (s, 3H), 7.21-7.30 (m, 4H). M.S. (calcd): 469.2 (MH⁺), M.S. (found): 469.0 (ESI) (MH⁺).

### Example 50

### 2-(4-acetylpiperazin-1-yl)-4-[2-(4-chlorobenzyl)pyrrolidin-1-yl]-6-isopropyl-5,6-dihydro-7H-pyrrolo[3,4-d]pyrimidin-7-one

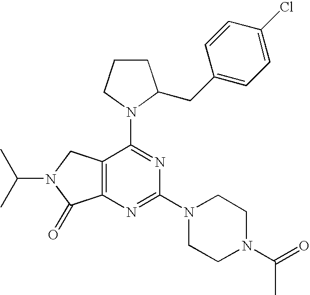

Following a procedure similar to that described in General Procedure 1 and after purification by reverse phase HPLC (gradient 5-15% CH₃CN in H₂O containing 0.1% trifluoroacetic acid) followed by preparative LCMS (gradient 35-55% CH₃CN in H₂O containing 0.1% trifluoroacetic acid) and lyophilization from CH₃CN/H₂O, the title compound (22 mg, 18%) was obtained as its TFA salt. HPLC: k′ 14.04; Purity: >96% (215 nm), >97% (254 nm), >99% (280 nm); Rₜ: 1.96 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.33 (t, J=5.86 Hz, 6H), 1.91 (s, 2H), 1.99-2.09 (m, 2H), 2.16 (s, 3H), 2.65 (s, 1H), 2.80 (dd, J=13.77, 8.89 Hz, 1H), 3.67-3.74 (m, 5H), 3.76-3.97 (m, 7H), 4.46-4.55 (m, 1H), 4.70 (s, 1H), 7.22 (d, J=8.40 Hz, 2H), 7.32 (d, J=8.40 Hz, 2H). M.S. (calcd): 497.2 (MH⁺), M.S. (found): 497.2 (ESI) (MH⁺). Found: C, 46.54; H, 5.00; N, 11.48. C₂₆H₃₃N₆O₂C1×1.9 CF₃CO₂H×2.9H₂O has C, 46.73; H, 5.36; N, 10.97%.

### Example 51

### 2-(4-acetylpiperazin-1-yl)-4-[benzyl(cyclopropylmethyl)amino]-6-isopropyl-5,6-dihydro-7H-pyrrolo[3,4-d]pyrimidin-7-one

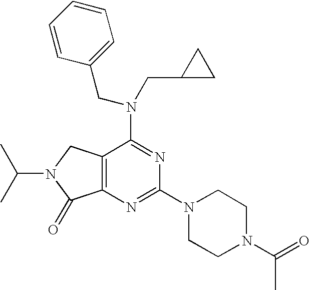

Following a procedure similar to that described in General Procedure 1 and after purification by reverse phase HPLC (gradient 45-65% CH₃CN in H₂O containing 10 mM NH₄HCO₃) and lyophilization from CH₃CN/H₂O, the resulting solid was dissolved in a 50% CH₃CN in H₂O containing 0.1% trifluoroacetic acid and concentrated under reduced pressure, the residue was lyophilized from CH₃CN/H₂O to provide the title compound (19 mg, 23%) as its TFA salt. HPLC: k′ 13.72; Purity: >93% (215 nm), >96% (254 nm), >92% (280 nm); Rₜ: 1.91 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 0.30-0.35 (m, 2H), 0.53-0.60 (m, 2H), 1.12-1.22 (m, 1H), 1.25 (d, J=6.84 Hz, 6H), 2.13 (s, 3H), 3.58-3.66 (m, 6H), 3.76-3.81 (m, 2H), 3.83-3.89 (m, 2H), 4.41-4.50 (m, 1H), 4.53 (s, 2H), 5.03 (s, 2H), 7.24-7.30 (m, 3H), 7.32-7.38 (m, 2H). M.S. (calcd): 463.3 (MH⁺), M.S. (found): 463.3 (ESI) (MH⁺). Found: C, 57.82; H, 6.26; N, 14.86. C₂₆H₃₄N₆O₂×0.9CF₃CO₂H×0.6H₂O has C, 57.97; H, 6.32; N, 14.59%.

### Example 52

### 2-(4-acetylpiperazin-1-yl)-4-[2-(4-chlorophenyl)pyrrolidin-1-yl]-6-isopropyl-5,6-dihydro-7H-pyrrolo[3,4-d]pyrimidin-7-one

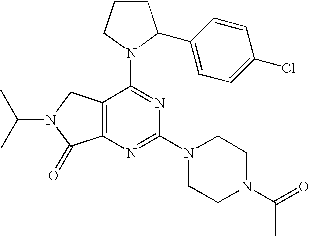

Following a procedure similar to that described in General Procedure 1 and after purification by silica gel chromatography (gradient 2-15% methanol:ethyl acetate) followed by preparative LCMS (gradient 35-55% CH₃CN in H₂O containing 0.1% trifluoroacetic acid) and lyophilization from CH₃CN/H₂O, the title compound (64 mg, 53%) was obtained as its TFA salt. HPLC: k′ 13.36; Purity: >91% (215 nm), >93% (254 nm), >99% (280 nm); Rₜ: 1.87 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (600 MHz, CD₃OD) δ ppm 1.31 (s, 3H), 1.32 (s, 3H), 1.56 (d, J=7.04 Hz, 4H), 2.12 (s, 3H), 3.42-3.59 (m, 5H), 3.63-3.83 (m, 5H), 4.29 (s, 2H), 4.48-4.53 (m, 1H), 5.25 (q, J=7.04 Hz, 1H), 7.31 (d, J=8.80 Hz, 2H), 7.36 (d, J=8.80 Hz, 2H). M.S. (calcd): 483.2 (MH⁺), M.S. (found): 483.3 (ESI) (MH⁺). Found: C, 50.78; H, 5.09; N, 12.70. C₂₅H₃₁N₆O₂Cl×1.5CF₃CO₂H×0.5H₂O has C, 50.72; H, 5.09; N, 12.67%.

### Example 53

### 2-(4-acetylpiperazin-1-yl)-4-{[1-(4-chlorophenyl)ethyl]amino}-6-isopropyl-5,6-dihydro-7H-pyrrolo[3,4-d]pyrimidin-7-one

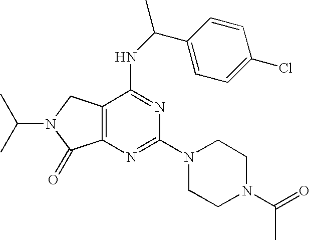

Following a procedure similar to that described in General Procedure 1 and after purification by silica gel chromatography (gradient 2-15% methanol:ethyl acetate) followed by preparative LCMS (gradient 35-55% CH₃CN in H₂O containing 0.1% trifluoroacetic acid) and lyophilization from CH₃CN/H₂O, the title compound (32 mg, 28%) was obtained as its TFA salt. HPLC: k′ 12.13; Purity: >99% (215 nm), >99% (254 nm), >99% (280 nm); Rₜ: 1.71 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.32 (d, J=6.64 Hz, 6H), 1.59 (d, J=7.23 Hz, 3H), 2.13 (s, 3H), 3.46-3.65 (m, 4H), 3.66-3.86 (m, 4H), 4.34 (s, 2H), 4.44-4.55 (m, 1H), 5.29 (q, J=7.03 Hz, 1H), 7.31-7.35 (m, 2H), 7.36-7.40 (m, 2H). M.S. (calcd): 457.2 (MH⁺), M.S. (found): 457.3 (ESI) (MH⁺). Found: C, 49.11; H, 4.94; N, 13.20. C₂₃H₂₉N₆O₂Cl×1.5CF₃CO₂H×0.4H₂O has C, 49.16; H, 4.97; N, 13.23%.

### Example 54

### 2-(4-acetylpiperazin-1-yl)-4-(2-benzylpyrrolidin-1-yl)-6-isopropyl-5,6-dihydro-7H-pyrrolo[3,4-d]pyrimidin-7-one

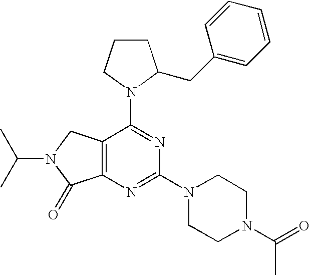

Following a procedure similar to that described in General Procedure 1 and after purification by silica gel chromatography (gradient 2-12% methanol:ethyl acetate) followed by preparative LCMS (gradient 35-55% CH₃CN in H₂O containing 0.1% trifluoroacetic acid) and lyophilization from CH₃CN/H₂O, the title compound (46 mg, 39%) was obtained as its TFA salt. HPLC: k′ 12.76; Purity: >91% (215 nm), >96% (254 nm), >95% (280 nm); Rₜ: 1.79 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (600 MHz, CD₃OD) δ ppm 1.32 (dd, J=6.16 Hz, 6H), 1.88-1.94 (m, 2H), 1.98-2.06 (m, 2H), 2.14 (s, 3H), 3.20 (d, J=10.56 Hz, 1H), 3.62-3.70 (m, 5H), 3.74-3.79 (m, 1H), 3.80-3.97 (m, 6H), 4.47-4.53 (m, 1H), 4.65-4.73 (m, 2H), 7.18-7.22 (m, 3H), 7.26-7.30 (m, 2H). M.S. (calcd): 463.3 (MH⁺), M.S. (found): 463.3 (ESI) (MH⁺). Found: C, 52.10; H, 5.29; N, 12.19. C₂₆H₃₄N₆O₂×2.0CF₃CO₂H has C, 52.17; H, 5.25; N, 12.17%.

### Example 55

### 2-(4-acetylpiperazin-1-yl)-4-{[2-(3,4-dimethylphenyl)ethyl]amino}-6-isopropyl-5,6-dihydro-7H-pyrrolo[3,4-d]pyrimidin-7-one

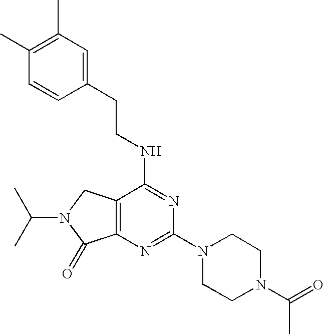

Following a procedure similar to that described in General Procedure 1 and after purification by silica gel chromatography (gradient 2-12% methanol:ethyl acetate) followed by preparative LCMS (gradient 45-65% CH₃CN in H₂O containing 0.1% trifluoroacetic acid) and lyophilization from CH₃CN/H₂O, the title compound (54 mg, 19%) was obtained as its TFA salt. HPLC: k′ 12.63; Purity: >99% (215 nm), >99% (254 nm), >99% (280 nm); Rₜ: 1.77 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (s, 3H), 1.31 (s, 3H), 2.15 (s, 3H), 2.20 (s, 3H), 2.21 (s, 3H), 2.87 (t, J=7.32 Hz, 2H), 3.62-3.76 (m, 6H), 3.83 (dd, J=6.44, 4.10 Hz, 2H), 3.87-3.92 (m, 2H), 4.20 (s, 2H), 4.43-4.55 (m, 1H), 6.91-6.95 (m, 1H), 6.98 (s, 1H), 7.02 (d, J=7.62 Hz, 1H). M.S. (calcd): 451.3 (MH⁺), M.S. (found): 451.2 (ESI) (MH⁺). Found: C, 58.06; H, 6.58; N, 15.25. C₂₅H₃₄N₆O₂×0.8 CF₃CO₂H×0.5H₂O has C, 58.00; H, 6.55; N, 15.26%.

### Example 56

### 2-(4-acetylpiperazin-1-yl)-4-{[2-(2,4-dimethylphenyl)ethyl]amino}-6-isopropyl-5,6-dihydro-7H-pyrrolo[3,4-d]pyrimidin-7-one

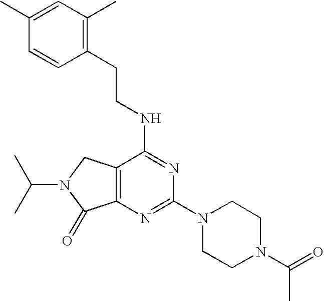

Following a procedure similar to that described in General Procedure 1 and after purification by silica gel chromatography (gradient 2-12% methanol: ethyl acetate) followed by preparative LCMS (gradient 45-65% CH₃CN in H₂O containing 10 mM NH₄HCO₃), the resulting solid was dissolved in acetonitrile containing 0.1% TFA and stirred for 1 h, concentrated under reduced pressure and lyophilized from CH₃CN/H₂O to give the title compound (54 mg, 19%) as its TFA salt. HPLC: k′ 12.68; Purity: >99% (215 nm), >99% (254 nm), >99% (280 nm); Rₜ: 1.78 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (s, 3H), 1.32 (s, 3H), 2.16 (s, 3H), 2.24 (s, 3H), 2.32 (s, 3H), 2.93 (t, J=7.43 Hz, 2H), 3.65-3.75 (m, 6H), 3.83 (dd, J=6.64, 4.10 Hz, 2H), 3.89 (dd, J=6.25, 4.10 Hz, 2H), 4.23 (s, 2H), 4.45-4.53 (m, 1H), 6.91 (d, J=7.81 Hz, 1H), 6.96-6.99 (m, 1H), 7.00 (d, J=7.81 Hz, 1H). M.S. (calcd): 451.3 (MH⁺), M.S. (found): 451.2 (ESI) (MH⁺). Found: C, 57.43; H, 6.69; N, 14.88. C₂₅H₃₄N₆O₂×0.8 CF₃CO₂H×0.8H₂O has C, 57.44; H, 6.60; N, 15.11%.

### Example 57

### 4-[2-(4-chlorobenzyl)pyrrolidin-1-yl]-2-{[2-(dimethylamino)ethyl]amino}-6-isopropyl-5,6-dihydro-7H-pyrrolo[3,4-d]pyrimidin-7-one

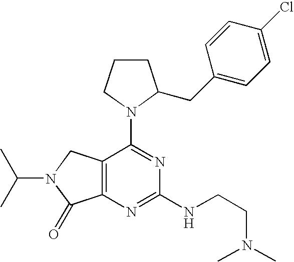

Following a procedure similar to that described in General Procedure 1 and after purification by preparative LCMS (gradient 55-75% CH₃CN in H₂O containing 10 mM NH₄HCO₃), the resulting solid was dissolved in acetonitrile containing 0.1% TFA and stirred for 1 h, concentrated under reduced pressure and lyophilized from CH₃CN/H₂O to give the title compound (37 mg, 16%) as its TFA salt. HPLC: k′ 11.13; Purity: >99% (215 nm), >99% (254 nm), >99% (280 nm); Rₜ: 1.58 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (600 MHz, CD₃OD, 320K) δ ppm 1.33 (t, J=6.46 Hz, 6H), 1.82-1.93 (m, 2H), 1.97-2.04 (m, 2H), 2.73 (dd, J=12.91, 8.80 Hz, 1H), 2.94 (s, 6H), 2.94-3.00 (m, 1H), 3.16 (d, J=9.39 Hz, 1H), 3.31-3.35 (m, 2H), 3.61-3.83 (m, 4H), 4.46-4.53 (m, 1H), 4.58-4.66 (m, 2H), 7.20-7.30 (m, 4H). M.S. (calcd): 457.3 (MH⁺), M.S. (found): 457.3 (ESI) (MH⁺). Found: C, 47.09; H, 5.14; N, 11.45. C₂₄H₃₃N₆OCl×2.3CF₃CO₂H×0.6H₂O has C, 47.05; H, 5.04; N, 11.51%.

### Example 58

### 2-(4-acetylpiperazin-1-yl)-4-[benzyl(4-chlorobenzyl)amino]-6-isopropyl-5,6-dihydro-7H-pyrrolo[3,4-d]pyrimidin-7-one

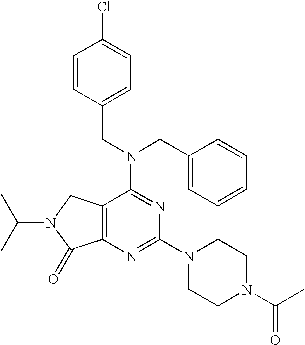

Following a procedure similar to that described in General Procedure 1 and purification by silica gel chromatography (gradient 5-30% methanol: ethyl acetate) followed by preparative LCMS (gradient 45-65% CH₃CN in H₂O containing 0.1% trifluoroacetic acid) and lyophilization from CH₃CN/H₂O, the title compound (30 mg, 15%) was obtained as a solid. HPLC: k′ 9.30; Purity: >99% (215 nm), >99% (254 nm), >99% (280 nm); Rₜ: 2.37 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.16 (s, 3H), 1.17 (s, 3H), 2.12 (s, 3H), 3.51-3.60 (m, 4H), 3.77 (dd, J=6.45, 4.10 Hz, 2H), 3.84 (dd, J=6.25, 4.30 Hz, 2H), 4.37 (s, 2H), 4.39-4.47 (m, 1H), 4.91 (d, J=2.15 Hz, 4H), 7.24-7.32 (m, 5H), 7.33-7.38 (m, 4H). M.S. (calcd): 533.2 (MH⁺), M.S. (found): 533.3 (ESI) (MH⁺). Found: C, 55.97; H, 5.25; N, 12.60. C₂₉H₃₃N₆O₂Cl×1.1CF₃CO₂H×0.6H₂O has C, 55.99; H, 5.32; N, 12.56%.

### Example 59

### 2-(4-acetylpiperazin-1-yl)-4-[(4-chlorobenzyl)(cyclopropylmethyl)amino]-6-isopropyl-5,6-dihydro-7H-pyrrolo[3,4-d]pyrimidin-7-one

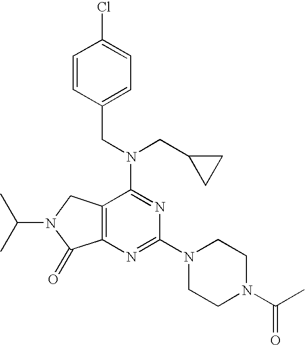

Following a procedure similar to that described in General Procedure 1 and purification by silica gel chromatography (gradient 5-30% methanol: ethyl acetate) followed by preparative LCMS (gradient 45-65% CH₃CN in H₂O containing 0.1% trifluoroacetic acid) and lyophilization from CH₃CN/H₂O, the title compound (47 mg, 36%) was obtained as a solid. HPLC: k′ 8.30; Purity: >92% (215 nm), >95% (254 nm), >96% (280 nm); Rₜ: 2.14 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN ¹H NMR (400 MHz, CD₃OD) δ ppm 0.30-0.35 (m, 2H), 0.54-0.60 (m, 2H), 1.11-1.20 (m, 1H), 1.28 (s, 3H), 1.29 (s, 3H), 2.13 (s, 3H), 3.56-3.65 (m, 6H), 3.75-3.79 (m, 2H), 3.81-3.86 (m, 2H), 4.43-4.52 (m, 1H), 4.58 (s, 2H), 5.02 (s, 2H), 7.25-7.29 (m, 2H), 7.33-7.38 (m, 2H). M.S. (calcd): 497.2 (MH⁺), M.S. (found): 497.2 (ESI) (MH⁺).

### Example 60

### 2-(4-acetylpiperazin-1-yl)-4-{[2-(4-chlorophenyl)-1,1-dimethylethyl]amino}-6-isopropyl-5,6-dihydro-7H-pyrrolo[3,4-d]pyrimidin-7-one

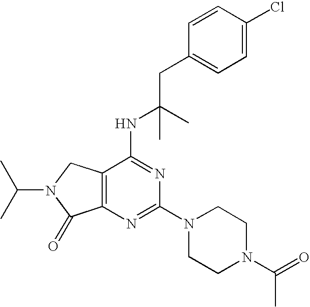

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by preparative LC/MS (gradient 45-65% CH₃CN in H₂O containing 0.1% trifluoroacetic acid) followed by lyophilization from CH₃CN/H₂O, the title compound (TFA salt) was obtained as a solid (233 mg, 76%). HPLC: k′ 7.59; Purity: >99% (215 nm), >99% (254 nm), >99% (280 nm); Rₜ: 1.98 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.50 (s, 6H), 2.16 (s, 3H), 3.28-3.32 (m, 2H), 3.72-3.78 (m, 4H), 3.90 (dd, J=5.66, 5.08 Hz, 2H), 3.97 (dd, J=5.47, 5.08 Hz, 2H), 4.21 (s, 2H), 4.43-4.54 (m, 1H), 7.05-7.10 (m, 2H), 7.22-7.26 (m, 2H). M.S. (calcd): 485.2 (MH⁺), M.S. (found): 485.3 (ESI) (MH⁺). Found: C, 50.55; H, 5.33; N, 12.72. C₂₅H₃₃N₆O₂Cl×1.5CF₃CO₂H×0.5H₂O has C, 50.57; H, 5.38; N, 12.64%.

### Example 61

### 4-{[2-(4-chlorophenyl)-1,1-dimethylethyl]amino}-2-[(2R,6S)-2,6-dimethylmorpholin-4-yl]-6-isopropyl-5,6-dihydro-7H-pyrrolo[3,4-d]pyrimidin-7-one

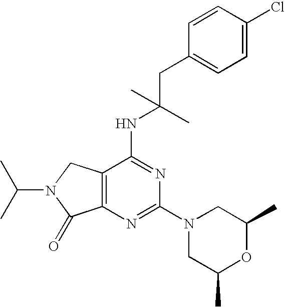

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 50-100% ethyl acetate:hexanes) followed by preparative LCMS (gradient 45-65% CH₃CN in H₂O containing 0.1% trifluoroacetic acid) and lyophilized from CH₃CN/H₂O, the title compound (TFA salt) was obtained as a solid (32 mg, 52%). HPLC: k′ 17.52; Purity: >99% (215 nm), >98% (254 nm), >99% (280 nm); Rₜ: 2.41 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.23 (s, 3H), 1.25 (s, 3H), 1.28 (s, 3H), 1.30 (s, 3H), 1.48 (s, 6H), 2.81 (dd, J=13.28, 10.74 Hz, 2H), 3.30 (s, 2H), 3.65-3.76 (m, 2H), 4.19 (s, 2H), 4.43-4.53 (m, 3H), 7.04-7.08 (m, 2H), 7.22-7.26 (m, 2H). M.S. (calcd): 472.2 (MH⁺), M.S. (found): 472.3 (ESI) (MH⁺). Found: C, 51.38; H, 5.54; N, 10.32. C₂₅H₃₄N₅O₂Cl×1.7CF₃CO₂H has C, 51.23; H, 5.40; N, 10.52%.

### Example 62

### N-[1-(4-{[2-(4-chlorophenyl)-1,1-dimethylethyl]amino}-6-isopropyl-7-oxo-6,7-dihydro-5H-pyrrolo[3,4-d]pyrimidin-2-yl)pyrrolidin-3-yl]acetamide

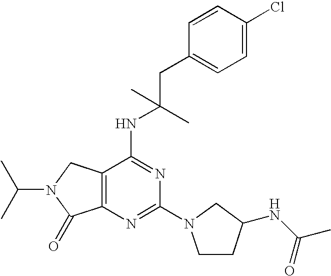

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 2-15% methanol: ethyl acetate) followed by preparative LCMS (gradient 35-55% CH₃CN in H₂O containing 0.1% trifluoroacetic acid) and lyophilized from CH₃CN/H₂O, the title compound (TFA salt) was obtained as a solid (33 mg, 58%). HPLC: k′ 13.37; Purity: >99% (215 nm), >99% (254 nm), >99% (280 nm); Rₜ: 1.87 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (600 MHz, CD₃OD) δ ppm 1.29 (s, 3H), 1.30 (s, 3H), 1.52 (s, 6H), 1.95 (s, 3H), 2.12 (s, 1H), 2.37 (s, 1H), 3.34 (s, 2H), 3.46-3.84 (m, 2H), 3.85-4.13 (m, 2H), 4.24 (s, 2H), 4.42-4.57 (m, 2H), 7.09 (d, J=8.22 Hz, 2H), 7.25 (d, J=8.80 Hz, 2H). M.S. (calcd): 485.2 (MH⁺), M.S. (found): 485.3 (ESI) (MH⁺). Found: C, 47.93; H, 4.99; N, 11.69. C₂₅H₃₃N₆O₂Cl×2.0CF₃CO₂H×0.7H₂O has C, 48.00; H, 5.06; N, 11.58%.

### Example 63

### 2-[4-(4-{[2-(4-chlorophenyl)-1,1-dimethylethyl]amino}-6-isopropyl-7-oxo-6,7-dihydro-5H-pyrrolo[3,4-d]pyrimidin-2-yl)piperazin-1-yl]-N,N-dimethylacetamide

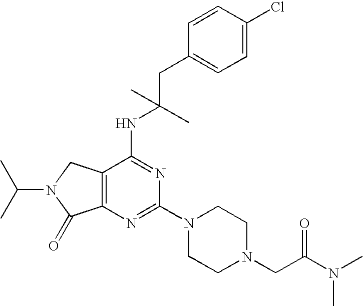

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by preparative LCMS (gradient 45-65% CH₃CN in H₂O containing 0.1% trifluoroacetic acid) and lyophilized from CH₃CN/H₂O, the title compound (TFA salt) was obtained as a solid (36 mg, 60%). HPLC: k′ 13.45; Purity: >99% (215 nm), >99% (254 nm), >99% (280 nm); Rₜ: 1.88 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.29 (s, 3H), 1.46 (s, 6H), 2.99 (s, 3H), 3.00 (s, 3H), 3.28-3.32 (m, 2H), 3.47 (br s, 8H), 4.12 (s, 2H), 4.28 (s, 2H), 4.47-4.56 (m, 1H), 7.00 (d, J=8.40 Hz, 2H), 7.21 (d, J=8.20 Hz, 2H). M.S. (calcd): 528.3 (MH⁺), M.S. (found): 528.3 (ESI) (MH⁺). Found: C, 46.83; H, 5.19; N, 11.95. C₂₇H₃₈N₇O₂Cl×2.4CF₃CO₂H×0.8H₂O has C, 46.80; H, 5.19; N, 12.01%.

### Example 64

### 4-{[2-(4-chlorophenyl)-1,1-dimethylethyl]amino}-2-(4-ethylpiperazin-1-yl)-6-isopropyl-5,6-dihydro-7H-pyrrolo[3,4-d]pyrimidin-7-one

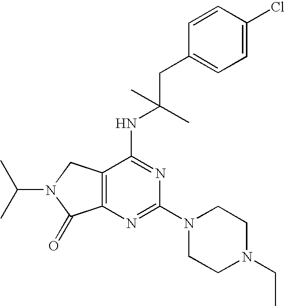

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by preparative LCMS (gradient 35-55% CH₃CN in H₂O containing 0.1% trifluoroacetic acid) and lyophilized from CH₃CN/H₂O, the title compound (TFA salt) was obtained as a solid (31 mg, 56%). HPLC: k′ 7.10; Purity: >99% (215 nm), >99% (254 nm), >99% (280 nm); Rₜ: 1.86 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.27 (s, 3H), 1.29 (s, 3H), 1.38 (t, J=7.32 Hz, 3H), 1.46 (s, 6H), 3.04-3.15 (m, 2H), 3.24 (q, J=7.42 Hz, 2H), 3.28-3.37 (m, 4H), 3.66 (d, J=11.13 Hz, 2H), 4.12 (s, 2H), 4.47-4.57 (m, 1H), 4.99 (d, J=13.28 Hz, 2H), 6.98-7.03 (m, 2H), 7.18-7.23 (m, 2H). M.S. (calcd): 471.3 (MH⁺), M.S. (found): 471.3 (ESI) (MH⁺).

### Example 65

### 2-(4-acetyl-1,4-diazepan-1-yl)-4-{[2-(4-chlorophenyl)-1,1-dimethylethyl]amino}-6-isopropyl-5,6-dihydro-7H-pyrrolo[3,4-d]pyrimidin-7-one

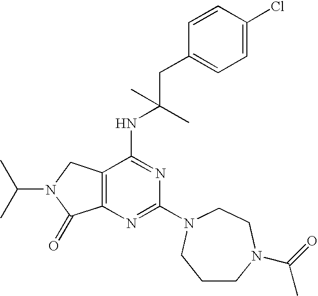

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol: ethyl acetate) followed by preparative LC/MS (gradient 35-55% CH₃CN in H₂O containing 0.1% trifluoroacetic acid) and lyophilized from CH₃CN/H₂O, the title compound (TFA salt) was obtained as a solid (36 mg, 61%). HPLC: k′ 7.52; Purity: >99% (215 nm), >99% (254 nm), >99% (280 nm); Rₜ: 1.96 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.48 (s, 3H), 1.50 (s, 3H), 1.90-2.04 (m, 2H), 2.06 (d, J=12.30 Hz, 3H), 3.28-3.32 (m, 2H), 3.59 (t, J=5.86 Hz, 1H), 3.64 (t, J=5.96 Hz, 1H), 3.81 (t, J=5.86 Hz, 1H), 3.86 (t, J=5.57 Hz, 1H), 3.88-4.11 (m, 4H), 4.18 (s, 1H), 4.21 (s, 1H), 4.43-4.54 (m, 1H), 7.02-7.09 (m, 2H), 7.21-7.27 (m, 2H). M.S. (calcd): 499.3 (MH⁺), M.S. (found): 499.3 (ESI) (MH⁺). Found: C, 49.54; H, 5.34; N, 11.70. C₂₆H₃₅N₆O₂Cl×1.8CF₃CO₂H×0.7H₂O has C, 49.59; H, 5.37; N, 11.72%.

### Example 66

### 4-{[2-(4-chlorophenyl)-1,1-dimethylethyl]amino}-6-isopropyl-2-{[3-(2-oxopyrrolidin-1-yl)propyl]amino}-5,6-dihydro-7H-pyrrolo[3,4-d]pyrimidin-7-one

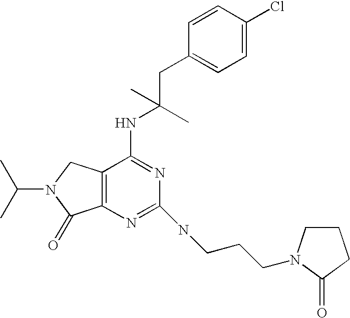

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol:ethyl acetate) followed by preparative LC/MS (gradient 35-55% CH₃CN in H₂O containing 0.1% trifluoroacetic acid) and lyophilized from CH₃CN/H₂O, the title compound (TFA salt) was obtained as a solid (33 mg, 61%). HPLC: k′ 7.61; Purity: >99% (215 nm), >99% (254 nm), >99% (280 nm); Rₜ: 1.98 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (s, 3H), 1.30 (s, 3H), 1.51 (s, 6H), 1.85-1.94 (m, 2H), 1.96-2.06 (m, 2H), 2.35 (t, J=8.11 Hz, 2H), 3.31-3.33 (m, 2H), 3.37-3.48 (m, 4H), 3.56 (t, J=6.15 Hz, 2H), 4.23 (s, 2H), 4.40-4.50 (m, 1H), 7.07-7.13 (m, 2H), 7.24-7.29 (m, 2H). M.S. (calcd): 499.3 (MH⁺), M.S. (found): 499.3 (ESI) (MH⁺). Found: C, 48.76; H, 5.45; N, 11.44. C₂₆H₃₅N₆O₂Cl×1.8CF₃CO₂H×1.4H₂O has C, 48.73; H, 5.47; N, 11.52%.

### Example 67

### N-[1-(4-{[2-(4-chlorophenyl)-1,1-dimethylethyl]amino}-6-isopropyl-7-oxo-6,7-dihydro-5H-pyrrolo[3,4-d]pyrimidin-2-yl)pyrrolidin-3-yl]-N-methylacetamide

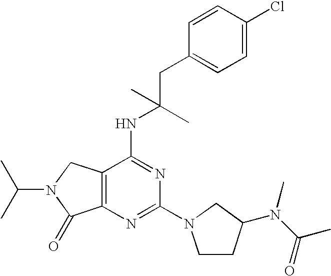

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol: ethyl acetate) followed by preparative LC/MS (gradient 45-65% CH₃CN in H₂O containing 0.1% trifluoroacetic acid) and lyophilized from CH₃CN/H₂O, the title compound (TFA salt) was obtained as a solid (16 mg, 28%). HPLC: k′ 7.65; Purity: >97% (215 nm), >98% (254 nm), >98% (280 nm); Rₜ: 1.99 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (s, 3H), 1.30 (s, 3H), 1.51 (s, 6H), 2.15 (s, 3H), 2.30 (br s, 2H), 3.05 (s, 3H), 3.31-3.36 (m, 2H), 3.56-3.80 (m, 2H), 3.97 (br s, 2H), 4.23 (s, 2H), 4.42-4.52 (m, 1H), 5.18 (br s, 1H), 7.07-7.11 (m, 2H), 7.23-7.28 (m, 2H). M.S. (calcd): 499.3 (MH⁺), M.S. (found): 499.3 (ESI) (MH⁺). Found: C, 48.47; H, 5.38; N, 11.19. C₂₆H₃₅N₆O₂Cl×2.0CF₃CO₂H×0.9H₂O has C, 48.48; H, 5.26; N, 11.31%.

### Example 68

### 4-{[2-(4-chlorophenyl)-1,1-dimethylethyl]amino}-6-isopropyl-2-(5-oxo-1,4-diazepan-1-yl)-5,6-dihydro-7H-pyrrolo[3,4-d]pyrimidin-7-one

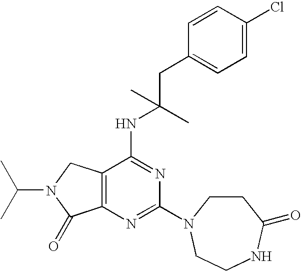

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol: ethyl acetate) followed by preparative LC/MS (gradient 45-65% CH₃CN in H₂O containing 0.1% trifluoroacetic acid) and lyophilized from CH₃CN/H₂O, the title compound (TFA salt) was obtained as a solid (35 mg, 46%). HPLC: k′ 7.83; Purity: >99% (215 nm), >99% (254 nm), >99% (280 nm); Rₜ: 2.03 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.48 (s, 6H), 2.74-2.79 (m, 2H), 3.29-3.31 (m, 2H), 3.44-3.47 (m, 2H), 4.04-4.12 (m, 4H), 4.19 (s, 2H), 4.45-4.53 (m, 1H), 7.05-7.09 (m, 2H), 7.22-7.26 (m, 2H). M.S. (calcd): 471.2 (MH⁺), M.S. (found): 471.3 (ESI) (MH⁺). Found: C, 49.87; H, 5.28; N, 12.77. C₂₄H₃₁N₆O₂Cl×1.5CF₃CO₂H×0.5H₂O has C, 49.81; H, 5.19; N, 12.91%.

### Example 69

### 4-{[2-(4-chlorophenyl)-1,1-dimethylethyl]amino}-6-isopropyl-2-(4-methyl-3-oxopiperazin-1-yl)-5,6-dihydro-7H-pyrrolo[3,4-d]pyrimidin-7-one

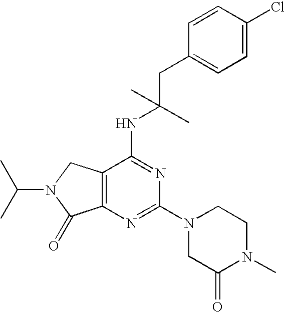

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol: ethyl acetate) followed by preparative LC/MS (gradient 35-55% CH₃CN in H₂O containing 0.1% trifluoroacetic acid) and lyophilized from CH₃CN/H₂O, the title compound (TFA salt) was obtained as a solid (24 mg, 32%). HPLC: k′ 7.00; Purity: >99% (215 nm), >99% (254 nm), >99% (280 nm); Rₜ: 1.84 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.29 (s, 3H), 1.49 (s, 6H), 3.03 (s, 3H), 3.32 (s, 2H), 3.55 (t, J=5.47 Hz, 2H), 4.12 (t, J=5.47 Hz, 2H), 4.16 (s, 2H), 4.43 (s, 2H), 4.45-4.55 (m, 1H), 7.03-7.07 (m, 2H), 7.20-7.24 (m, 2H). M.S. (calcd): 471.2 (MH⁺), M.S. (found): 471.3 (ESI) (MH⁺). Found: C, 53.23; H, 5.67; N, 14.06. C₂₄H₃₁N₆O₂Cl×1.0CF₃CO₂H×0.1H₂O has C, 53.22; H, 5.53; N, 14.32%.

### Example 70

### 4-{[2-(4-chlorophenyl)-1,1-dimethylethyl]amino}-6-isopropyl-2-{[2-(2-oxopyrrolidin-1-yl)ethyl]amino}-5,6-dihydro-7H-pyrrolo[3,4-d]pyrimidin-7-one

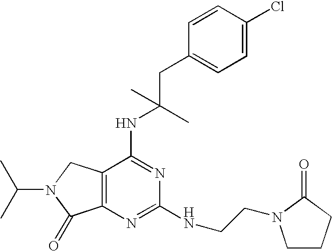

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol:ethyl acetate) followed by preparative LC/MS (gradient 35-55% CH₃CN in H₂O containing 0.1% trifluoroacetic acid) and lyophilized from CH₃CN/H₂O, the title compound (TFA salt) was obtained as a solid (32 mg, 41%). HPLC: k′ 7.30 Purity: >99% (215 nm), >99% (254 nm), >99% (280 nm); Rₜ: 1.91 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.53 (s, 6H), 2.01-2.10 (m, 2H), 2.36 (t, J=8.11 Hz, 2H), 3.33 (s, 2H), 3.53 (t, J=7.03 Hz, 2H), 3.60 (t, J=5.76 Hz, 2H), 3.72 (t, J=5.66 Hz, 2H), 4.23 (s, 2H), 4.40-4.49 (m, 1H), 7.10-7.15 (m, 2H), 7.24-7.28 (m, 2H). M.S. (calcd): 485.2 (MH⁺), M.S. (found): 485.2 (ESI) (MH⁺). Found: C, 49.89; H, 5.35; N, 12.78. C₂₅H₃₃N₆O₂Cl×1.4CF₃CO₂H×1.3H₂O has C, 49.98; H, 5.58; N, 12.58%.

### Example 71

### 4-{[2-(4-chlorophenyl)-1,1-dimethylethyl]amino}-6-isopropyl-2-(4-propionylpiperazin-1-yl)-5,6-dihydro-7H-pyrrolo[3,4-d]pyrimidin-7-one

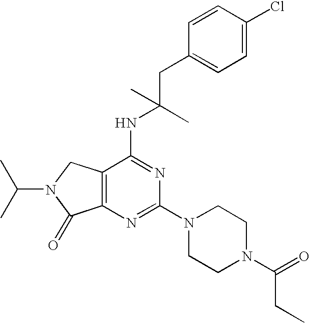

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol:ethyl acetate) followed by preparative LC/MS (gradient 45-65% CH₃CN in H₂O containing 0.1% trifluoroacetic acid) and lyophilized from CH₃CN/H₂O, the title compound (TFA salt) was obtained as a solid (26 mg, 33%). HPLC: k′ 8.17 Purity: >97% (215 nm), >96% (254 nm), >96% (280 nm); Rₜ: 2.11 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.13 (t, J=7.42 Hz, 3H), 1.28 (s, 3H), 1.30 (s, 3H), 1.49 (s, 6H), 2.47 (q, J=7.42 Hz, 2H), 3.28-3.32 (m, 2H), 3.69-3.77 (m, 4H), 3.89 (dd, J=6.54, 4.00 Hz, 2H), 3.95 (dd, J=6.35, 4.20 Hz, 2H), 4.18 (s, 2H), 4.45-4.54 (m, 1H), 7.03-7.08 (m, 2H), 7.21-7.26 (m, 2H). M.S. (calcd): 499.3 (MH⁺), M.S. (found): 499.2 (ESI) (MH⁺). Found: C, 52.02; H, 5.69; N, 13.29. C₂₆H₃₅N₆O₂Cl×1.2CF₃CO₂H×1.0H₂O has C, 52.17; H, 5.89; N, 12.85%.

### Example 72

### 2-(4-Acetyl-piperazin-1-yl)-4-(benzhydryl-amino)-6-isopropyl-5,6-dihydro-pyrrolo[3,4-d]pyrimidin-7-one

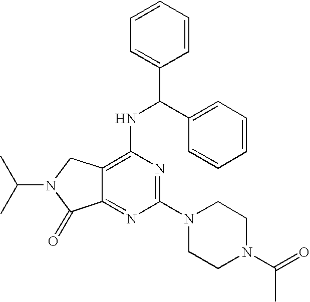

Following a procedure similar to that described in General procedure 3, starting from 2-(4-Acetyl-piperazin-1-yl)-4-(benzhydryl-amino)-5H-furo[3,4-d]pyrimidin-7-one (Intermediate 96) and after preparative HPLC purification (0.1% TFA/H₂O/CH₃CN), the title compound was isolated as a solid (0.09 g, 23%). HPLC: 98.1%. M.S. (calcd): 484.6 (MH⁺), M.S. (found): 485 (ESI) (MH⁺). Found: C, 69.19; H, 6.74; N, 17.07. C₂₈H₃₂N₆O₂ has C, 69.4; H, 6.66; N, 17.34.

### Example 73

### 4-(Benzhydryl-amino)-6-isobutyl-2-morpholin-4-yl-5,6-dihydro-pyrrolo[3,4-d]pyrimidin-7-one

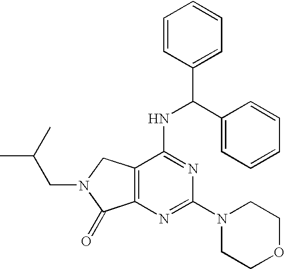

Following a procedure similar to that described in General procedure 3, starting from 4-(benzhydryl-amino)-2-morpholin-4-yl-5H-furo[3,4-d]-pyrimidin-7-one (Intermediate 95) and after preparative HPLC purification (0.1% TFA/H₂O/CH₃CN), the title compound was isolated as a solid (34 mg, 8.0%). HPLC purity>98.3%. M.S. (calcd): 457.6 (MH⁺), M.S. (found): 458 (ESI) (MH⁺). Found: C, 65.05; H, 6.45; N, 13.75. C₂₇H₃₁N₅O₂×0.6 (CH₂Cl₂) has C, 65.19; H, 6.38; N, 13.77.

### Example 74

### 4-(Benzhydryl-amino)-6-cyclopropyl-2-morpholin-4-yl-5,6-dihydro-pyrrolo[3,4-d]pyrimidin-7-one

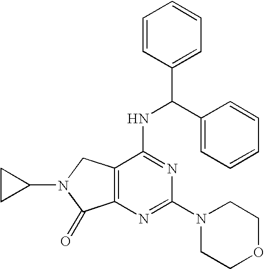

Following a procedure similar to that described in General procedure 3, starting from 4-(benzhydryl-amino)-2-morpholin-4-yl-5H-furo[3,4-d]-pyrimidin-7-one (Intermediate 95) and after preparative HPLC purification (0.1% TFA/H₂O/CH₃CN), the title compound was isolated as a solid (24 mg, 11%). HPLC purity>98.0%. M.S. (calcd): 441.5 (MH⁺), M.S. (found): 442 (ESI) (MH⁺). Found: C, 69.79; H, 6.03; N, 15.30. C₂₆H₂₇N₅O₂×0.33H₂O has C, 69.78; H, 6.23; N, 15.65.

### Example 75

### 4-(Benzhydryl-amino)-6-cyclopentyl-2-morpholin-4-yl-5,6-dihydro-pyrrolo[3,4-d]pyrimidin-7-one

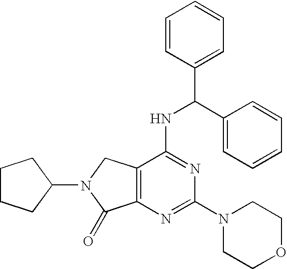

Following a procedure similar to that described in General procedure 3, starting from 4-(benzhydryl-amino)-2-morpholin-4-yl-5H-furo[3,4-d]-pyrimidin-7-one (Intermediate 95) and after preparative HPLC purification (0.1% TFA/H₂O/CH₃CN), the title compound was isolated as a solid (0.027 g, 8.0%). HPLC purity>95.0%. M.S. (calcd): 469.6 (MH⁺), M.S. (found): 470 (ESI) (MH⁺). Found: C, 70.64; H, 6.72; N, 13.97. C₂₈H₃₁N₅O₂×0.4H₂O has C, 70.53; H, 6.72; N, 14.69.

### Example 76

### 6-isopropyl-2-morpholin-4-yl-[(phenyl-p-tolyl-methyl)-amino]-5,6-dihydro-pyrrolo[3,4-d]pyrimidin-7-one

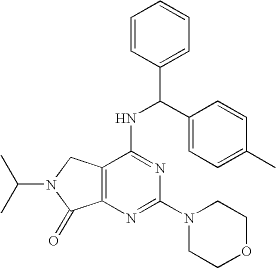

Following a procedure similar to that described in General procedure 3, starting from 2-morpholin-4-yl-4-[(phenyl-p-tolyl-methyl-amino]-5H-furo[3,4-d]pyrimidin-7-one (Intermediate 98) and after preparative HPLC purification (0.1% TFA/H₂O/CH₃CN), the title compound was isolated as a solid (0.026 g, 11%). HPLC purity>95.0%. M.S. (calcd): 457.6 (MH⁺), M.S. (found): 458 (ESI) (MH⁺). Found: C, 69.11; H, 6.98; N, 13.99. C₂₇H₃₁N₅O₂×0.66H₂O has C, 69.06; H, 6.94; N, 14.91.

### Example 77

### 4-{[(4-Chloro-phenyl)-phenyl-methyl]-amino}-6-isopropyl-2-morpholin-4-yl-5,6-dihydro-pyrrolo[3,4-d]pyrimidin-7-one

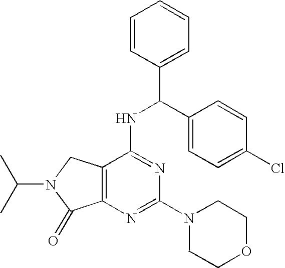

Following a procedure similar to that described in General procedure 3, starting from 4-{[(4-chloro-phenyl)-phenyl-methyl]amino}-2-morpholin-4-yl-5H-furo[3,4-d]pyrimidin-7-one (Intermediate 97) and after preparative HPLC purification (0.1% TFA/H₂O/CH₃CN), the title compound was isolated as a solid (0.074 g, 19%). HPLC purity>98.2%. ¹H NMR (400 MHz, CD₃OD) δ ppm 1.24 (d, J=6.63 Hz, 6H), 3.59 (t, J=4.29 Hz, 4H), 3.67 (br s, 4H), 4.09 (s, 2H), 4.62-4.72 (m, 1H), 4.96 (d, J=5.85 Hz, 1H), 6.30 (d, J=5.85 Hz, 1H), 7.22-7.39 (m, 9H). M.S. (calcd): 478.0 (MH⁺), M.S. (found): 477 (ESI) (MH⁺). Found: C, 64.48; H, 5.59; N, 14.15. C₂₆H₂₈ClN₅O₂×0.33H₂O has C, 64.52; H, 5.97; N, 14.47.

### Example 78

### 4-(Benzhydryl-amino)-2-(4-ethyl-piperazin-1-yl)-6-isopropyl-5,6-dihydro-pyrrolo[3,4-d]pyrimidin-7-one

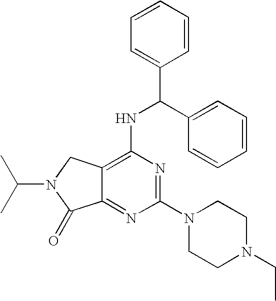

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (0.09 g, 60%). HPLC: 95.3%. M.S. (calcd): 470.6 (MH⁺), M.S. (found): 471 (ESI) (MH⁺). Found: C, 69.66; H, 7.25; N, 17.19. C₂₈H₃₄N₆O×0.67H₂O has C, 69.19; H, 7.04; N, 17.40.

### Example 79

### 4-(Benzhydryl-amino)-2-(4-cyclopropanecarbonyl-piperazin-1-yl)-6-isopropyl-5,6-dihydro-pyrrolo[3,4-d]pyrimidin-7-one

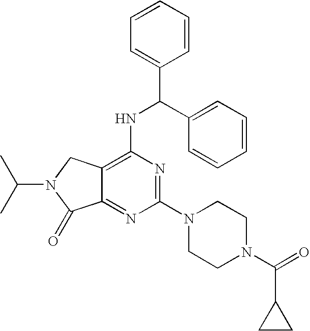

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (0.11 g, 69%). HPLC: 96.7%. ¹H NMR (400 MHz, CD₃OD) δ ppm 0.76-0.81 (m, 2H), 0.97-1.03 (m, 2H), 1.26 (d, J=6.74 Hz, 6H), 1.70-1.77 (m, 1H), 3.48-3.70 (m, 6H), 3.80-3.87 (m, 2H), 4.11 (s, 2H), 4.64-4.73 (m, 1H), 5.05 (d, J=5.86 Hz, 1H), 6.31 (d, J=5.86 Hz, 1H), 7.29-7.40 (m, 10H). M.S. (calcd): 510.6 (MH⁺), M.S. (found): 511 (ESI) (MH⁺). Found: C, 69.34; H, 6.63; N, 16.01. C₃₀H₃₄N₆O₂×0.5H₂O has C, 69.36; H, 6.55; N, 16.18.

### Example 80

### 4-(Benzhydryl-amino)-2-(4-isobutyryl-piperazin-1-yl)-6-isopropyl-5,6-dihydro-pyrrolo[3,4-d]pyrimidin-7-one

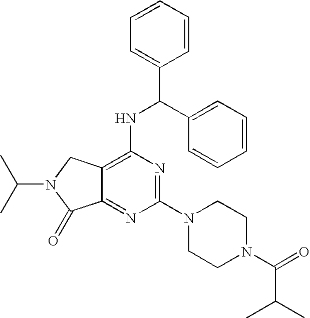

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after recrystallization from EtOAc: hexanes, the title compound was obtained as a solid (0.11 g, 71%). HPLC: 95.4%. M.S. (calcd): 512.7 (MH⁺), M.S. (found): 513 (ESI) (MH⁺). Found: C, 69.01; H, 7.16; N, 16.04. C₃₀H₃₆N₆O₂×0.5H₂O has C, 69.01; H, 6.9; N, 16.1.

### Example 81

### 4-(Benzhydryl-amino)-6-isopropyl-2-morpholin-4-yl-5,6-dihydro-pyrrolo[3,4-d]pyrimidin-7-one

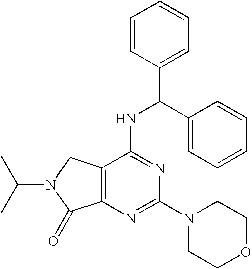

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after preparative HPLC purification (0.1% TFA/H₂O/CH₃CN), the title compound was obtained as a solid (100 mg). HPLC: 95.0%. M.S. (calcd): 443.6 (MH⁺), M.S. (found): 444 (ESI) (MH⁺). Found: C, 70.38; H, 6.45; N, 15.43. C₂₆H₂₉N₅O₂ has C, 70.41; H, 6.59; N, 15.79.

### Example 82

### 4-(Benzhydryl-amino)-6-dimethylamino-2-(4-methyl-piperazin-1-yl)-5,6-dihydro-cyclopentapyrimidin-7-one

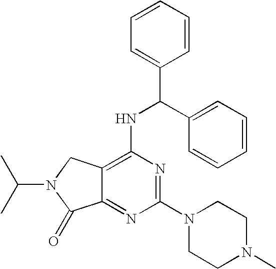

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (40 mg, 27%). HPLC: 95.5%. M.S. (calcd): 456.6 (MH⁺), M.S. (found): 457 (ESI) (MH⁺). Found: C, 70.97; H, 7.01; N, 17.78. C₂₇H₃₂N₆O has C, 71.03; H, 7.06; N, 18.41.

### Example 83

### 4-(Benzhydryl-amino)-6-isopropyl-2-piperazin-1-yl-5,6-dihydro-pyrrolo[3,4-d]pyrimidin-7-one

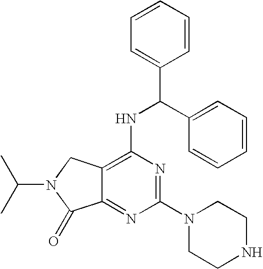

HCl (1.0 mL of concentrated aqueous solution) was added drop wise to a solution of 1-[4-(benzhydryl-amino)-6-isopropyl-7-oxo-6,7-dihydro-5H-pyrrolo[3,4-d]pyrimidin-2-yl]-piperidine-4-carboxylic acid tert-butyl ester (Intermediate 117) (0.12 g, 0.22 mmol) in dichloromethane (3.6 mL). After 30 minutes the reaction mixture was concentrated under reduced pressure to give the title compound (HCl salt) as a solid (60 mg, 62%). HPLC: 95.3%. M.S. (calcd): 442.6 (MH⁺), M.S. (found): 443 (ESI) (MH⁺). Found: C, 58.63; H, 6.40; N, 15.63. C₂₆H₃₀N₆O×2.5HCl has C, 58.51; H, 6.09; N, 15.75.

### Example 84

### 4-(Benzhydryl-amino)-2-benzylamino-6-isopropyl-5,6-dihydro-pyrrolo[3,4-d]pyrimidin-7-one

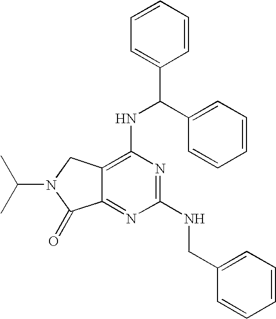

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after purification by silica gel chromatography (10% EtOAc in hexanes), the title compound was obtained as a solid (50 mg, 43%). HPLC: 95.4%. ¹H NMR (400 MHz, CD₃OD) δ ppm 1.28 (d, J=6.73 Hz, 6H), 4.22 (s, 2H), 4.46 (s, 2H), 4.45-4.53 (m, 1H), 6.52 (s, 1H), 7.17-7.32 (m, 15H). M.S. (calcd): 463.6 (MH⁺), M.S. (found): 464 (ESI) (MH⁺). Found: C, 74.20; H, 6.42; N, 14.13. C₂₉H₂₉N₅O×0.08H₂O has C, 74.80; H, 6.24; N, 15.05.

### Example 85

### 4-(Benzhydryl-amino)-6-isopropyl-2-(2-methoxy-ethylamino)-5,6-dihydro-pyrrolo[3,4-d]pyrimidin-7-one

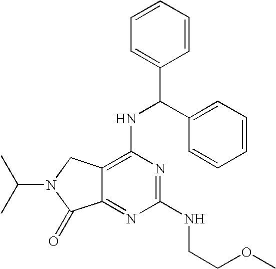

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after purification by silica gel chromatography (1% MeOH in EtOAc), the title compound was obtained as a solid (70 mg, 55%). HPLC: 96%. ¹H NMR (400 MHz, CD₃OD) δ ppm 1.29 (d, J=6.73 Hz, 6H), 3.25 (s, 3H), 3.29-3.34 (m, 2H), 3.40-3.45 (m, 2H), 4.23 (s, 2H), 4.45-4.55 (m, 1H), 6.56 (s, 1H), 7.22-7.29 (m, 2H), 7.29-7.37 (m, 8H). M.S. (calcd): 431.5 (MH⁺), M.S. (found): 432 (ESI) (MH⁺). Found: C, 69.63; H, 7.03; N, 15.54. C₂₂H₂₂N₄O₃ has C, 69.58; H, 6.77; N, 16.23.

### Example 86

### 4-(Benzhydryl-amino)-2-(2,6-dimethyl-morpholin-4-yl)-6-isopropyl-5,6-dihydro-pyrrolo[3,4-d]pyrimidin-7-one

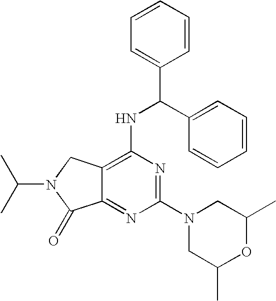

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after recrystallization from EtOAc: hexanes, the title compound was obtained as a solid (0.11 g, 79%). HPLC: 97.2%. ¹H NMR (400 MHz, CD₃OD) δ ppm 1.12 (d, J=6.44 Hz, 6H), 1.29 (d, J=6.73 Hz, 6H), 2.38 (dd, J=12.88, 10.83 Hz, 2H), 3.33-3.44 (m, 2H), 4.25 (s, 2H), 4.46 (d, J=13.47 Hz, 2H), 4.46-4.56 (m, 1H), 6.36 (s, 1H), 7.21-7.29 (m, 2H), 7.32 (d, J=4.68 Hz, 8H). M.S. (calcd): 471.6 (MH⁺), M.S. (found): 472 (ESI) (MH⁺). Found: C, 70.99; H, 7.15; N, 14.59. C₂₅H₂₆N₄O₃ has C, 71.31; H, 7.05; N, 14.85.

### Example 87

### 4-(Benzhydryl-amino)-6-isopropyl-2-(4-propionyl-piperazin-1-yl)-5,6-dihydro-pyrrolo[3,4-d]pyrimidin-7-one

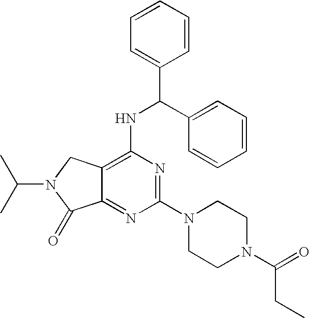

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after trituration with EtOAc, the title compound was obtained as a solid (90 mg, 59%). HPLC: 96.4%. ¹H NMR (400 MHz, CDCl₃) δ ppm 1.15 (t, J=7.32 Hz, 3H), 1.25 (d, J=6.73 Hz, 6H), 2.35 (q, J=7.22 Hz, 2H), 3.33 (m, 2H), 3.47 (m, 2H), 3.63 (m, 2H), 3.76 (m, 2H), 4.09 (s, 2H), 4.63-4.70 (m, 1H), 5.04 (d, J=5.85 Hz, 1H), 6.29 (d, J=6.15 Hz, 1H), 7.28-7.37 (m, 10H). M.S. (calcd): 498.6 (MH⁺), M.S. (found): 499 (ESI) (MH⁺). Found: C, 69.33; H, 6.86; N, 16.47. C₂₉H₃₄N₆O₂×0.08H₂O has C, 69.59; H, 6.79; N, 16.79.

### Example 88

### 2-(4-Acetyl-piperazin-1-yl)-4-(1,2-diphenyl-ethylamino)-6-isopropyl-5,6-dihydro-pyrrolo[3,4-d]pyrimidin-7-one

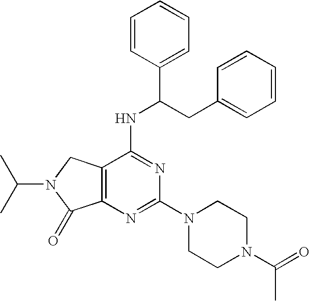

Following a procedure similar to that described in General Procedure 4, starting from 2-chloro-4-(1,2-diphenyl-ethylamino)-6-isopropyl-5,6-dihydro-pyrrolo[3,4-d]pyrimidin-7-one (Intermediate 103) and after purification by silica gel chromatography (acetone), the title compound was obtained as a solid (123 mg, 56%). HPLC: 96.1%. ¹H NMR (400 MHz, CD₃OD) δ ppm 1.24 (d, J=6.63 Hz, 6H), 2.11 (s, 3H), 3.20 (d, J=7.02 Hz, 2H), 3.32-3.39 (m, 2H), 3.43-3.52 (m, 1H), 3.54-3.62 (m, 1H), 3.65-3.72 (m, 2H), 3.72-3.80 (m, 1H), 3.82-3.88 (m, 1H), 3.93 (d, J=16.00 Hz, 1H), 4.04 (d, J=16.00 Hz, 1H), 4.63-4.70 (m, 1H), 4.81-4.89 (m, 1H), 5.32 (q, J=6.90 Hz, 1H), 7.10 (d, J=6.64 Hz, 2H), 7.22-7.33 (m, 8H). M.S. (calcd): 498.6 (MH⁺), M.S. (found): 499 (ESI) (MH⁺). Found: C, 69.36; H, 7.13; N, 16.37. C₂₉H₃₄N₆O₂×0.2H₂O has C, 69.35; H, 6.90; N, 16.73.

### Example 89

### 4-(Benzhydryl-amino)-2-diethylamino-6-isopropyl-5,6-dihydro-pyrrolo[3,4-d]pyrimidin-7-one

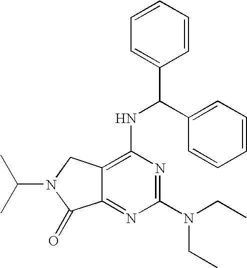

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (60 mg, 90%). HPLC: 95.4%. ¹H NMR (400 MHz, CD₃OD) δ ppm 0.95 (br s, 6H), 1.20 (d, J=6.73 Hz, 6H), 3.48 (br s, 4H), 4.11 (s, 2H), 4.56-4.66 (m, 1H), 5.28 (d, J=6.15 Hz, 1H), 6.38 (d, J=6.15 Hz, 1H), 7.22-7.33 (m, 10H). M.S. (calcd): 429.6 (MH⁺), M.S. (found): 430 (ESI) (MH⁺). Found: C, 72.39; H, 7.63; N, 15.79. C₂₆H₃₁N₅O has C, 72.70; H, 7.27; N, 16.30.

### Example 90

### 2-{4-[4-(Benzhydryl-amino)-6-isopropyl-7-oxo-6,7-dihydro-5H-pyrrolo[3,4-d]pyrimidin-2-yl]-piperazin-1-yl}-N,N-dimethyl-acetamide

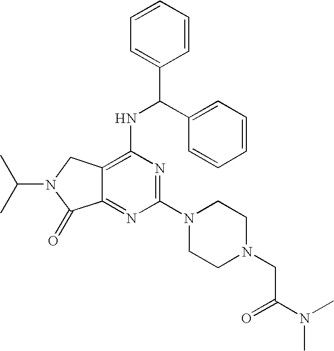

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after purification by silica gel chromatography (4% MeOH in CH₂Cl₂), the title compound was obtained as a solid (0.12 g, 74%). HPLC: 97.7%. ¹H NMR (400 MHz, CD₃OD) δ ppm 1.17 (d, J=6.73 Hz, 6H), 2.32-2.41 (m, 4H), 2.93 (s, 3H), 3.06 (s, 3H), 3.09 (s, 2H), 3.68 (s, 4H), 4.16 (s, 2H), 4.54-4.63 (m, 1H), 5.81 (d, J=6.15 Hz, 1H), 6.36 (d, J=6.15 Hz, 1H), 7.22 (d, J=6.44 Hz, 2H), 7.24-7.32 (m, 8H). M.S. (calcd): 527.7 (MH⁺), M.S. (found): 528 (ESI) (MH⁺). Found: C, 66.41; H, 7.01; N, 17.52. C₃₀H₃₇N₇O₂×1.0H₂O has C, 66.05; H, 6.78; N, 17.98.

### Example 91

### 2-(4-acetylpiperazin-1-yl)-4-[(2,2-diphenylethyl)amino]-6-isopropyl-5,6-dihydro-7H-pyrrolo[3,4-d]pyrimidin-7-one

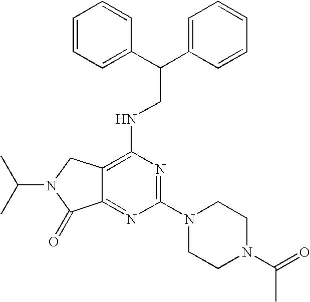

Following a procedure similar to that described in General Procedure 4, starting from 2-chloro-4-(2,2-diphenyl-ethylamino)-6-isopropyl-5,6-dihydro-pyrrolo[3,4-d]pyrimidin-7-one (Intermediate 100) and after recrystallization from EtOAc: hexanes, the title compound was obtained as a solid (0.13 g, 87%). HPLC: 95.6%. ¹H NMR (400 MHz, CD₃OD) δ ppm 1.22 (d, J=6.73 Hz, 6H), 2.15 (s, 3H), 3.47-3.53 (m, 2H), 3.65-3.71 (m, 2H), 3.85-3.90 (m, 4H), 3.92-3.97 (m, 2H), 4.14 (t, J=6.88 Hz, 2H), 4.36 (t, J=7.90 Hz, 1H), 4.44-4.49 (m, 1H), 4.60-4.69 (m, 1H), 7.16-7.39 (m, 10H). M.S. (calcd): 498.6 (MH⁺), M.S. (found): 499 (ESI) (MH⁺). Found: C, 69.93; H, 6.90; N, 16.04. C₂₉H₃₄N₆O₂ has C, 69.86; H, 6.87; N, 16.85.

### Example 92

### 4-(Benzhydryl-amino)-6-isopropyl-2-(4-methanesulfonyl-piperazin-1-yl)-5,6-dihydro-pyrrolo[3,4-d]pyrimidin-7-one

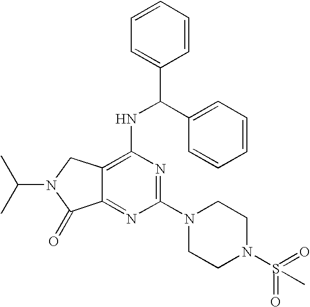

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after trituration with MeOH, the title compound was obtained as a solid (0.13 g, 81%). HPLC: 96.5%. ¹H NMR (400 MHz, DMSO-d₆) δ ppm 1.20 (d, J=6.74 Hz, 6H), 2.81 (s, 3H), 2.98 (br s, 4H), 3.74 (br s, 4H), 4.21 (s, 2H), 4.33-4.40 (m, 1H), 6.43 (d, J=7.04 Hz, 1H), 7.26 (t, J=7.04 Hz, 2H), 7.32-7.42 (m, 8H), 8.29 (d, J=7.33 Hz, 1H). M.S. (calcd): 520.7 (MH⁺), M.S. (found): 521 (ESI) (MH⁺). Found: C, 61.46; H, 6.19; N, 15.72. C₂₇H₃₂N₆O₃S×0.33H₂O has C, 61.59; H, 6.08; N, 15.96.

### Example 93

### N-{2-[4-(Benzhydryl-amino)-6-isopropyl-7-oxo-6,7-dihydro-5H-pyrrolo[3,4-d]pyrimidin-2-ylamino]-ethyl}-acetamide

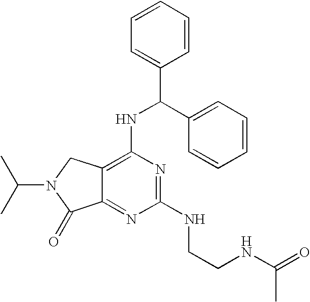

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (0.12 g, 85%). HPLC: 96.01%. ¹H NMR (400 MHz, DMSO-d₆) δ ppm 1.20 (d, J=6.73 Hz, 6H), 1.79 (s, 3H), 3.11 (s, 2H), 3.22 (s, 2H), 4.18 (s, 2H), 4.31-4.42 (m, 1H), 6.62 (d, J=8.20 Hz, 1H), 6.77 (s, 1H), 7.22-7.29 (m, 2H), 7.30-7.43 (m, 8H), 7.80 (s, 1H), 8.09 (s, 1H). M.S. (calcd): 458.6 (MH⁺), M.S. (found): 459 (ESI) (MH⁺). Found: C, 66.30; H, 6.39; N, 17.55. C₂₆H₃₀N₆O₂×0.67H₂O has C, 66.38; H, 6.38; N, 17.87.

### Example 94

### 4-(Benzhydryl-amino)-6-isopropyl-2-[(pyridin-3-ylmethyl)-amino]-5,6-dihydro-pyrrolo[3,4-d]pyrimidin-7-one

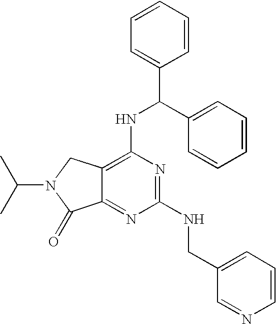

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after purification by silica gel chromatography (4% MeOH in CH₂Cl₂), the title compound was obtained as a solid (80 mg, 58%). HPLC: 97.4%. ¹H NMR (400 MHz, DMSO-d₆) δ ppm 1.18 (d, J=6.63 Hz, 6H), 4.16 (s, 2H), 4.30-4.35 (m, 1H), 4.40 (br s, 2H), 6.47 (br s, 1H), 7.19-7.35 (m, 10H), 7.44 (br s, 1H), 7.53 (br s, 1H), 8.09 (d, J=8.20 Hz, 1H), 8.39 (d, J=4.68 Hz, 1H), 8.48 (s, 1H). M.S. (calcd): 464.6 (MH⁺), M.S. (found): 465 (ESI) (MH⁺). Found: C, 72.45; H, 6.14; N, 17.49. C₂₈H₂₈N₆O has C, 72.39; H, 6.01; N, 18.09.

### Example 95

### 2-(4-Acetyl-piperazin-1-yl)-4-dibenzylamino-6-isopropyl-5,6-dihydro-pyrrolo[3,4-d]-pyrimidin-7-one

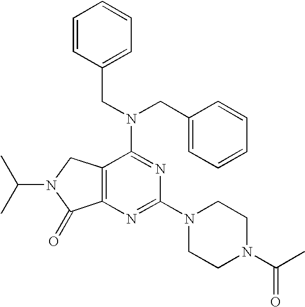

Following a procedure similar to that described in General Procedure 4, starting from 2-chloro-4-dibenzylamino-6-isopropyl-5,6-dihydro-pyrrolo[3,4-d]pyrimidin-7-one (Intermediate 102) and after purification by silica gel chromatography (5% MeOH in CH₂Cl₂), the title compound was isolated as a solid (98 mg, 40%). HPLC: 97.3%. ¹H NMR (400 MHz, CD₃OD) δ ppm 1.12 (d, J=6.63 Hz, 6H), 2.11 (s, 3H), 3.41-3.47 (m, 2H), 3.56-3.62 (m, 2H), 3.80 (br s, 2H), 3.89 (br s, 2H), 4.15 (s, 2H), 4.54-4.63 (m, 1H), 4.78 (s, 4H), 7.22 (d, J=7.42 Hz, 4H), 7.28-7.38 (m, 6H). M.S. (calcd): 498.6 (MH⁺), M.S. (found): 499 (ESI) (MH⁺). Found: C, 70.21; H, 6.87; N, 16.44. C₂₉H₃₄N₆O₂ has C, 69.86; H, 6.87; N, 16.85.

### Example 96

### N-{1-[4-(Benzhydryl-amino)-6-isopropyl-7-oxo-6,7-dihydro-5H-pyrrolo[3,4-d]pyrimidin-2-yl]-pyrrolidin-3-yl}-acetamide

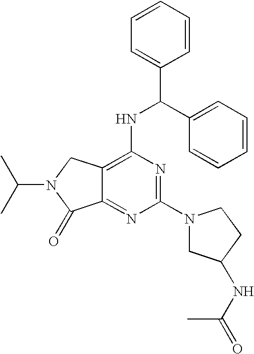

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after recrystallization from EtOAc: hexanes, the title compound was obtained as a solid (50 mg, 36%). HPLC: 95.8%. ¹H NMR (400 MHz, CD₃OD) δ ppm 1.19 (dd, J=6.44, 3.22 Hz, 6H), 1.85 (dddd, J=12.00, 5.93, 5.78, 5.56 Hz, 1H), 1.95 (s, 3H), 2.11 (td, J=13.10, 7.17 Hz, 1H), 3.31 (dd, J=11.71, 3.81 Hz, 1H), 3.50 (br s, 2H), 3.56 (dd, J=12.00, 6.15 Hz, 1H), 4.18 (d, J=3.51 Hz, 2H), 4.42 (br s, 1H), 4.54-4.63 (m, 1H), 5.71 (d, J=6.73 Hz, 1H), 6.11 (d, J=7.03 Hz, 1H), 6.49 (d, J=6.73 Hz, 1H), 7.21-7.32 (m, 10H). M.S. (calcd): 484.6 (MH⁺), M.S. (found): 485 (ESI) (MH⁺).

### Example 97

### 4-(Benzhydryl-amino)-2-(2-dimethylamino-ethylamino)-6-isopropyl-5,6-dihydro-pyrrolo[3,4-d]pyrimidin-7-one

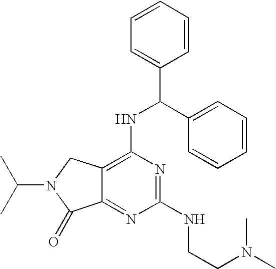

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and after purification by silica gel chromatography (gradient 5-25% MeOH in CH₂Cl₂), the title compound was obtained as a solid (50 mg, 96%). HPLC: 95.8%. ¹H NMR (400 MHz, CD₃OD) δ ppm 1.28 (d, J=6.74 Hz, 6H), 2.21 (s, 6H), 2.42 (br s, 2H), 3.43 (t, J=5.57 Hz, 2H), 4.24 (s, 2H), 4.50 (m, 1H), 6.63 (s, 1H), 7.21-7.29 (m, 2H), 7.29-7.34 (m, 8H). M.S. (calcd): 444.6 (MH⁺), M.S. (found): 445 (ESI) (MH⁺).

### Example 98

### 6-isopropyl-2-(2-methoxy-ethylamino)-4-[(phenyl-p-tolyl-methyl)-amino]-5,6-dihydro-pyrrolo[3,4-d]-pyrimidin-7-one

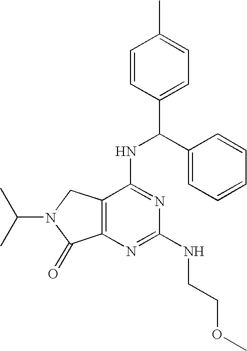

Following a procedure similar to that described in General Procedure 4, starting from 2-chloro-6-isopropyl-4-[(phenyl-p-tolyl-methyl)-amino]-5,6-dihydro-pyrrolo[3,4-d]pyrimidin-7-one (Intermediate 101) and after purification by silica gel chromatography (100% MeOH in CH₂Cl₂), the title compound was isolated (67 mg, 61%) as a solid. HPLC: 96.1%. ¹H NMR (400 MHz, CD₃OD) δ ppm 1.23 (d, J=6.63 Hz, 6H), 2.35 (s, 3H), 3.27 (br s, 5H), 3.45 (br s, 2H), 4.07 (s, 2H), 4.63-4.73 (m, 1H), 5.01 (br s, 1H), 5.40 (br s, 1H), 6.38 (d, J=5.07 Hz, 1H), 7.13-7.21 (m, 4H), 7.23-7.37 (m, 5H). M.S. (calcd): 445.6 (MH⁺), M.S. (found): 446 (ESI) (MH⁺). Found: C, 70.06; H, 7.22; N, 15.02. C₂₆H₃₁N₅O₂×0.1EtOAc has C, 69.79; H, 7.05; N, 15.41.

### Example 99

### 4-(Benzhydryl-amino)-2-[4-(2-dimethylamino-acetyl)-piperazin-1-yl]-6-isopropyl-5,6-dihydro-pyrrolo[3,4-d]pyrimidin-7-one

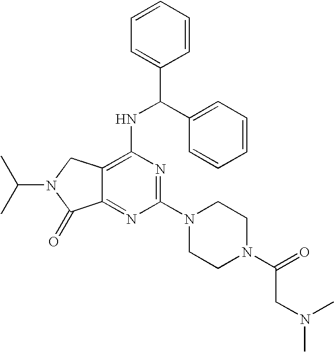

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99) and 4-(2-dimethylamino-acetyl)-piperazine hydrochloride (Intermediate 76) and after purification by silica gel chromatography (gradient 5-10% MeOH in CH₂Cl₂), the title compound was obtained as a solid (60 mg, 74%). HPLC: 96.8%. ¹H NMR (400 MHz, CD₃OD) δ ppm 1.22 (d, J=6.74 Hz, 6H), 2.27 (s, 6H), 3.08 (s, 2H), 3.41-3.49 (m, 4H), 3.63 (br s, 2H), 3.73 (br s, 2H), 4.13 (s, 2H), 4.56-4.70 (m, 1H), 5.42 (d, J=6.16 Hz, 1H), 6.32 (d, J=6.16 Hz, 1H), 7.24-7.37 (m, 10H). M.S. (calcd): 527.7 (MH⁺), M.S. (found): 528 (ESI) (MH⁺).

### Example 100

### 4-(Benzhydryl-amino)-6-dimethylamino-2-(2-hydroxy-ethylamino)-5,6-dihydro-cyclopentapyrimidin-7-one

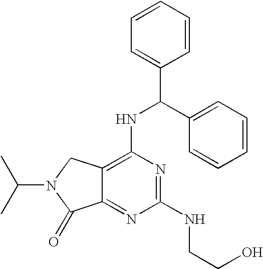

Following a procedure similar to that described in General Procedure 4, starting from 2-chloro-6-isopropyl-4-{[(4-methoxy-phenyl)-phenyl-methyl]-amino}-5,6-dihydro-pyrrolo[3,4-d]pyrimidin-7-one (Intermediate 99), the title compound was obtained as a solid (60 mg, 96%). HPLC: 95.4%. ¹H NMR (400 MHz, CD₃OD) δ ppm 1.24 (d, J=7.02 Hz, 6H), 3.42 (br s, 2H), 3.56 (br s, 2H), 4.09 (s, 2H), 4.62-4.71 (m, 1H), 5.06 (d, J=6.24 Hz, 1H), 5.56 (br s, 1H), 6.34 (s, 1H), 7.26-7.37 (m, 10H). M.S. (calcd): 417.5 (MH⁺), M.S. (found): 418 (ESI) (MH⁺).

### Example 101

### 2-(4-Ethyl-piperazin-1-yl)-6-isopropyl-4-[(phenyl-p-tolyl-methyl)-amino]-5,6-dihydro-pyrrolo[3,4-d]-pyrimidin-7-one

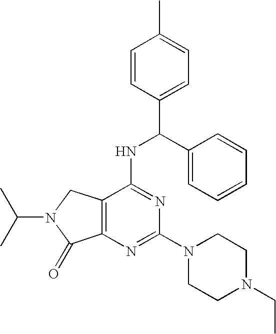

Following a procedure similar to that described in General Procedure 4, starting from 2-chloro-6-isopropyl-4-[(phenyl-p-tolyl-methyl)-amino]-5,6-dihydro-pyrrolo[3,4-d]pyrimidin-7-one (Intermediate 101) and after purification by silica gel chromatography (5% MeOH in CH₂Cl₂), the title compound was isolated (57 mg, 49%). HPLC: 94.1%. ¹H NMR (400 MHz, CDCl₃) δ ppm 1.10 (t, J=7.22 Hz, 3H), 1.23 (d, J=6.63 Hz, 6H), 2.31-2.43 (m, 6H), 2.34 (s, 3H), 3.74 (br s, 4H), 4.05 (s, 2H), 4.61-4.71 (m, 1H), 4.96 (d, J=5.46 Hz, 1H), 6.30 (d, J=5.85 Hz, 1H), 7.12-7.22 (m, 4H), 7.27-7.36 (m, 5H). M.S. (calcd): 484.7 (MH⁺), M.S. (found): 485 (ESI) (MH⁺). Found: C, 71.93; H, 7.58; N, 16.89. C₂₉H₃₆N₆O×0.11EtOAc has C, 71.53; H, 7.52; N, 17.00.

### Example 102

### 2-(4-isopropyl-piperazin-1-yl)-6-isopropyl-4-[(phenyl-p-tolyl-methyl)-amino]-5,6-dihydro-pyrrolo[3,4-d]-pyrimidin-7-one

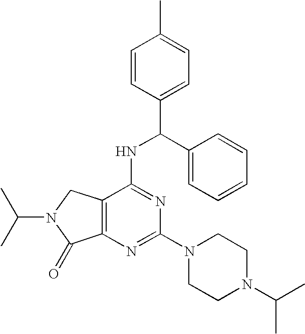

Following a procedure similar to that described in General Procedure 4, starting from 2-chloro-6-isopropyl-4-[(phenyl-p-tolyl-methyl)-amino]-5,6-dihydro-pyrrolo[3,4-d]pyrimidin-7-one (Intermediate 101) and after purification by silica gel chromatography (10% MeOH in CH₂Cl₂), the title compound was isolated (42 mg, 40%). HPLC: 96.4%. ¹H NMR (400 MHz, CD₃OD) δ ppm 1.04 (d, J=6.26 Hz, 6H), 1.23 (d, J=6.65 Hz, 6H), 2.35 (s, 3H), 2.40 (br s, 4H), 2.65 (br s, 1H), 3.73 (br s, 4H), 4.06 (s, 2H), 4.67 (m, 1H), 4.96 (d, J=5.09 Hz, 1H), 6.31 (d, J=6.26 Hz, 1H), 7.14-7.22 (m, 4H), 7.28-7.36 (m, 5H). M.S. (calcd): 498.7 (MH⁺), M.S. (found): 499 (ESI) (MH⁺). Found: C, 71.39; H, 7.65; N, 16.11. C₃₀H₃₈N₆O×0.235EtOAc×0.019CH₂Cl₂ has C, 71.37; H, 7.72; N, 16.13.

### Example 103

### 2-(4-Acetyl-piperazin-1-yl)-6-isopropyl-4-{[(4-methoxy-phenyl)-phenyl-methyl]-amino}-5,6-dihydro-pyrrolo[3,4-d]pyrimidin-7-one

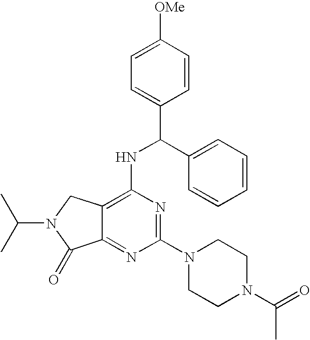

Following a procedure similar to that described in General Procedure 4, starting from 2-chloro-6-isopropyl-4-{[(4-methoxyphenyl)(phenyl)methyl]amino}-5,6-dihydro-7H-pyrrolo[3,4-d]pyrimidin-7-one (Intermediate 106) and after purification by silica gel chromatography (gradient 5-10% MeOH in CH₂Cl₂), the title compound was obtained as a solid (80 mg, 55%). HPLC: 97.7%. ¹H NMR (400 MHz, CDCl₃) δ ppm 1.24 (dd, J=6.73, 2.34 Hz, 6H), 2.10 (s, 3H), 3.34 (t, J=4.68 Hz, 2H), 3.44-3.53 (m, 2H), 3.65 (br s, 2H), 3.78 (br s, 2H), 3.80 (s, 3H), 4.08 (s, 2H), 4.62-4.71 (m, 1H), 5.00 (d, J=4.68 Hz, 1H), 6.25 (d, J=5.85 Hz, 1H), 6.88 (d, J=8.78 Hz, 2H), 7.22 (d, J=8.49 Hz, 2H), 7.27-7.37 (m, 5H). M.S. (calcd): 514.6 (MH⁺), M.S. (found): 515 (ESI) (MH⁺).

### Example 104

### 2-(4-Acetyl-piperazin-1-yl)-4-(4-chloro-benzylamino)-6-isopropyl-5,6-dihydro-pyrrolo[3,4-d]-pyrimidin-7-one

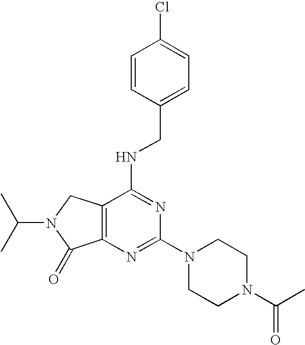

Following a procedure similar to that described in General Procedure 4, starting from 2-chloro-4-(4-chloro-benzyl)-6-isopropyl-5,6-dihydro-pyrrolo[3,4-d]pyrimidin-7-one (Intermediate 104) and after purification by silica gel chromatography (5% MeOH in CH₂Cl₂), the title compound was isolated as a solid (76 mg, 35%). HPLC: 95.5%. ¹H NMR (400 MHz, CDCl₃) δ ppm 1.25 (d, J=7.02 Hz, 6H), 2.14 (s, 3H), 3.43-3.49 (m, 2H), 3.61-3.67 (m, 2H), 3.80-3.86 (m, 2H), 3.88-3.93 (m, 2H), 4.09 (s, 2H), 4.63-4.71 (m, 1H), 4.68 (d, J=5.85 Hz, 2H), 4.94 (br s, 1H), 7.27-7.34 (m, 4H). M.S. (calcd): 443.0 (MH⁺), M.S. (found): 443 (ESI) (MH⁺). Found: C, 58.78; H, 6.15; N, 17.08. C₂₂H₂₇ClN₆O₂×0.5EtOAc×0.2H₂O has C, 58.76; H, 6.45; N, 17.13.

### Example 105

### 2-(4-Acetyl-piperazin-1-yl)-6-isopropyl-4-(3-isopropyl-phenyl amino)-5,6-dihydro-pyrrolo[3,4-d]-pyrimidin-7-one

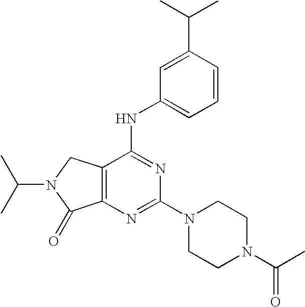

Following a procedure similar to that described in General Procedure 4, starting from 2-chloro-6-isopropyl-4-(3-isopropyl-phenylamino)-5,6-dihydro-pyrrolo[3,4-d]pyrimidin-7-one (Intermediate 105) and after purification by silica gel chromatography (5% MeOH in CH₂Cl₂), the title compound was isolated as a solid (91 mg, 71%). HPLC: 98.33%. ¹H NMR (400 MHz, CD₃OD) δ ppm 1.23 (dd, J=6.65, 1.96 Hz, 6H), 1.28 (dd, J=6.85, 2.15 Hz, 6H), 2.16 (s, 3H), 2.89-2.98 (m, 1H), 3.51 (br s, 2H), 3.69 (br s, 2H), 3.89 (br s, 2H), 3.96 (br s, 4H), 4.62-4.71 (m, 1H), 6.55 (br s, 1H), 7.09 (d, J=7.43 Hz, 1H), 7.25-7.35 (m, 2H), 7.42 (br s, 1H). M.S. (calcd): 436.6 (MH⁺), M.S. (found): 437 (ESI) (MH⁺). Found: C, 66.38; H, 7.41; N, 18.42. C₂₄H₃₂N₆O₂ has C, 66.03; H, 7.39; N, 19.25.

### Example 106

### 4-Dibenzylamino-2-(2-dimethylamino-ethylamino)-6-isopropyl-5,6-dihydro-pyrrolo[3,4-d]-pyrimidin-7-one

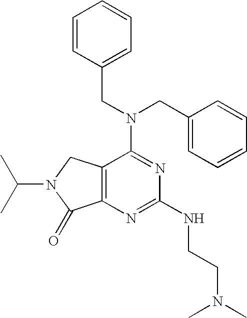

Following a procedure similar to that described in General Procedure 4, starting from 2-chloro-4-dibenzylamino-6-isopropyl-5,6-dihydro-pyrrolo[3,4-d]pyrimidin-7-one (Intermediate 102) and after purification by silica gel chromatography (20% MeOH in CH₂Cl₂), the title compound was isolated as a solid (0.042 g, 30%). HPLC: 95.1%. ¹H NMR (400 MHz, CDCl₃) δ ppm 1.10 (d, J=6.63 Hz, 6H), 2.19 (s, 6H), 2.44 (br s, 2H), 3.47 (br s, 2H), 4.13 (s, 2H), 4.56-4.66 (m, 1H), 4.78 (s, 4H), 5.62 (br s, 1H), 7.22 (d, J=7.02 Hz, 4H), 7.27-7.38 (m, 6H). M.S. (calcd): 458.6 (MH⁺), M.S. (found): 459 (ESI) (MH⁺). Found: C, 70.62; H, 7.85; N, 17.54. C₂₇H₃₄N₆O×0.1EtOAc has C, 70.41; H, 7.50; N, 17.98.

### Example 107

### 4-(2,2-Diphenyl-ethylamino)-2-(4-ethyl-piperazin-1-yl)-6-isopropyl-5,6-dihydro-pyrrolo[3,4-d]pyrimidin-7-one

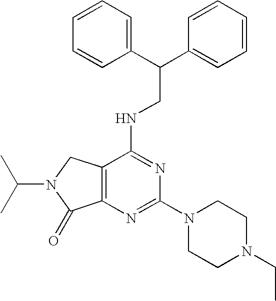

Following a procedure similar to that described in General Procedure 4, starting from 2-chloro-4-(2,2-diphenyl-ethylamino)-6-isopropyl-5,6-dihydro-pyrrolo[3,4-d]pyrimidin-7-one (Intermediate 100) and after recrystallization from EtOAc: hexanes, the title compound was obtained as a solid (140 mg, 83%). HPLC: 96.7%. ¹H NMR (400 MHz, CDCl₃) δ ppm 1.13 (t, J=7.03 Hz, 3H), 1.20 (d, J=6.73 Hz, 6H), 2.48 (br s, 6H), 3.84 (s, 2H), 3.92 (br s, 4H), 4.13 (t, J=7.03 Hz, 2H), 4.36 (t, J=7.47 Hz, 2H), 4.57-4.68 (m, 1H), 7.24-7.30 (m, 6H), 7.30-7.38 (m, 4H). M.S. (calcd): 484.7 (MH⁺), M.S. (found): 485 (ESI) (MH⁺). Found: C, 71.78; H, 7.60; N, 17.01. C₂₉H₃₆N₆O has C, 71.87; H, 7.49; N, 17.34.

### Example 108

### 2-(3-Dimethylamino-propylamino)-4-(2,2-diphenyl-ethylamino)-6-isopropyl-5,6-dihydro-pyrrolo[3,4-d]pyrimidin-7-one

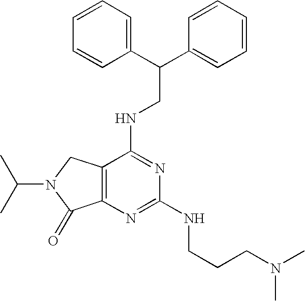

Following a procedure similar to that described in General Procedure 4, starting from 2-chloro-4-(2,2-diphenyl-ethylamino)-6-isopropyl-5,6-dihydro-pyrrolo[3,4-d]pyrimidin-7-one (Intermediate 100) and after recrystallization from EtOAc: hexanes, the title compound was obtained as a solid (118 mg, 69%). HPLC: 95.9%. ¹H NMR (400 MHz, CD₃OD) δ ppm 1.21 (d, J=6.74 Hz, 6H), 1.78 (t, J=4.84 Hz, 2H), 2.23 (s, 6H), 2.37 (br s, 2H), 3.54 (q, J=5.77 Hz, 2H), 3.86 (s, 2H), 4.15 (t, J=6.45 Hz, 2H), 4.40 (t, 2H), 4.61-4.71 (m, 1H), 5.55 (br s, 1H), 7.26-7.30 (m, 6H), 7.32-7.38 (m, 4H). M.S. (calcd): 472.6 (MH⁺), M.S. (found): 473 (ESI) (MH⁺). Found: C, 71.22; H, 7.83; N, 17.66. C₂₈H₃₆N₆O has C, 71.16; H, 7.68; N, 17.78.

### Example 109

### 4-(2,2-Diphenyl-ethylamino)-6-isopropyl-2-(2-methylamino-ethylamino)-5,6-dihydro-pyrrolo[3,4-d]pyrimidin-7-one

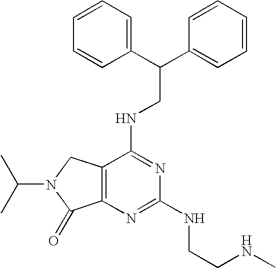

HCl (1 mL, 4M solution in dioxane) was added to a solution of {2-[4-(2,2-diphenylethylamino)-6-isopropyl-7-oxo-6,7-dihydro-5H-pyrrolo[3,4-d]pyrimidin-2-ylamino]-ethyl}-methyl-carbamic acid tert-butyl ester (Intermediate 119) (0.21 mmol) in anhydrous dioxane (3 mL). The solution was stirred at rt for 18 h. The reaction mixture was diluted with ether (15 mL) and the precipitate filtered under vacuum then washed with cold ether. The product was recrystallized from EtOAc and hexanes with a few drops of MeOH to give the title compound as a solid (88 mg, 77%). HPLC: 96.2%. ¹H NMR (400 MHz, CD₃OD) δ ppm 1.28 (d, J=6.73 Hz, 6H), 2.69 (s, 3H), 3.27 (t, J=5.86 Hz, 2H), 3.83 (t, J=5.42 Hz, 2H), 4.19 (s, 2H), 4.27 (d, J=7.32 Hz, 2H), 4.39-4.49 (m, 2H), 7.23 (dt, J=6.07, 2.96 Hz, 2H), 7.29-7.35 (m, 8H). M.S. (calcd): 444.6 (MH⁺), M.S. (found): 445 (ESI) (MH⁺). Found: C, 57.40; H, 6.11; N, 14.97. C₂₆H₃₂N₆O×2.75HCl has C, 57.32; H, 6.43; N, 15.42.

### Example 110

### 4-[(diphenylmethyl)amino]-2-morpholin-4-yl-6-propyl-5,6-dihydro-7H-pyrrolo[3,4-d]pyrimidin-7-one

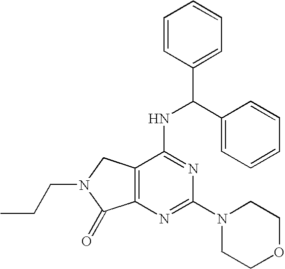

Following a procedure similar to that described in General procedure 3, starting from 4-(benzhydryl-amino)-2-morpholin-4-yl-5H-furo[3,4-d]-pyrimidin-7-one (Intermediate 95) and after purification by reverse phase HPLC (gradient 45-65% CH₃CN in H₂O containing 10 mM NH₄HCO₃, followed by CH₃CN in H₂O containing 0.1% trifluoroacetic acid) followed by lyophilization in CH₃CN/H₂O, the title compound (8 mg, 7%) was obtained as a solid. HPLC: k′ 15.03; Purity: >99% (215 nm), >99% (254 nm), >99% (280 nm); Rₜ: 2.08 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 0.89 (t, J=7.03 Hz, 3H), 1.60 (q, J=6.05 Hz, 2H), 2.77 (br s, 3H), 3.41-3.48 (m, 1H), 3.61-3.66 (m, 4H), 3.66-3.72 (m, 4H), 4.29 (s, 1H), 6.29 (d, J=5.66 Hz, 1H), 7.27-7.38 (m, 10H). M.S. (calcd): 444.2 (MH⁺), M.S. (found): 443.8 (ESI) (MH⁺).

### Example 111

### 2-(4-acetylpiperazin-1-yl)-4-[(4-chlorobenzyl)(methyl)amino]-6-isopropyl-5,6-dihydro-7H-pyrrolo[3,4-d]pyrimidin-7-one

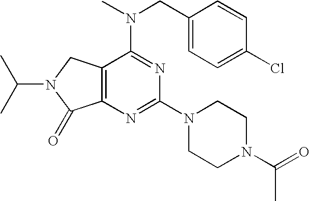

Following a procedure similar to that described in General Procedure 1 and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (40 mg, 54%) was obtained as its TFA salt. HPLC: k′ 12.61; Purity: >99% (215 nm), >99% (254 nm), >99% (280 nm); Rₜ: 1.77 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.64 Hz, 6H), 2.11 (s, 3H), 3.32 (s, 3H), 3.54-3.66 (m, 4H), 3.73-3.89 (m, 4H), 4.41-4.55 (m, 1H), 4.67 (s, 2H), 4.93 (s, 2H), 7.22-7.38 (m, 4H). M.S. (calcd): 457.2 (MH⁺), M.S. (found): 457.3 (ESI) (MH⁺).

### Example 112

### 2-(4-acetylpiperazin-1-yl)-6-isopropyl-4-({(1R)-1-[4-(trifluoromethoxy)phenyl]ethyl}amino)-5,6-dihydro-7H-pyrrolo[3,4-d]pyrimidin-7-one

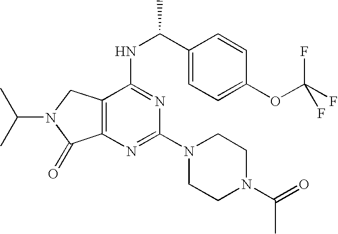

Following a procedure similar to that described in General Procedure 1 and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (105 mg, 64%) was obtained as its TFA salt. HPLC: k′ 13.69; Purity: >98% (215 nm), >98% (254 nm), >99% (280 nm); Rₜ: 1.91 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (dd, J=6.64, 1.95 Hz, 6H), 1.60 (d, J=7.03 Hz, 3H), 2.09-2.15 (m, 3H), 3.47-3.87 (m, 8H), 4.37 (s, 2H), 4.41-4.50 (m, 1H), 5.35 (q, J=7.03 Hz, 1H), 7.23 (d, J=8.20 Hz, 2H), 7.49 (d, J=8.59 Hz, 2H). M.S. (calcd): 507.2 (MH⁺), M.S. (found): 507.2 (ESI) (MH⁺).

### Example 113

### 2-(4-acetylpiperazin-1-yl)-4-({[1-(4-chlorophenyl)cyclopentyl]methyl}amino)-6-isopropyl-5,6-dihydro-7H-pyrrolo[3,4-d]pyrimidin-7-one

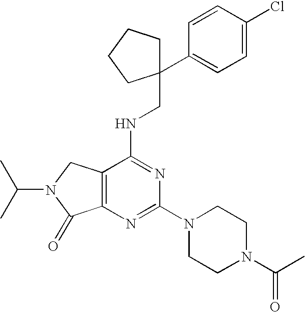

Following a procedure similar to that described in General Procedure 1 and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (68 mg, 33%) was obtained as its TFA salt. HPLC: k′ 15.23; Purity: >97% (215 nm), >99% (254 nm), >99% (280 nm); Rₜ: 2.11 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.64 Hz, 6H), 1.65-1.99 (m, 6H), 2.04-2.13 (m, 2H), 2.14 (s, 3H), 3.59-3.71 (m, 6H), 3.71-3.80 (m, 4H), 4.21 (s, 2H), 4.36-4.49 (m, 1H), 7.18-7.24 (m, 2H), 7.25-7.34 (m, 2H). M.S. (calcd): 511.2 (MH⁺), M.S. (found): 511.2 (ESI) (MH⁺).

### Example 114

### 2-(4-acetylpiperazin-1-yl)-4-({(1R)-1-[4-(difluoromethoxy)phenyl]ethyl}amino)-6-isopropyl-5,6-dihydro-7H-pyrrolo[3,4-d]pyrimidin-7-one

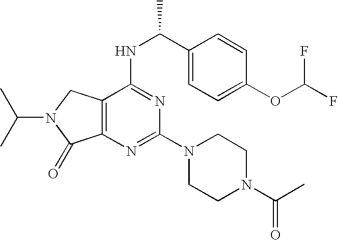

Following a procedure similar to that described in General Procedure 1 and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (79 mg, 42%) was obtained as its TFA salt. HPLC: k′ 11.77; Purity: >98% (215 nm), >98% (254 nm), >97% (280 nm); Rₜ: 1.66 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.22 (dd, J=6.64, 1.56 Hz, 6H), 1.50 (d, J=7.03 Hz, 3H), 2.02 (s, 3H), 3.37-3.58 (m, 4H), 3.59-3.77 (m, 4H), 4.27 (s, 2H), 4.32-4.43 (m, 1H), 5.23 (q, J=7.03 Hz, 1H), 6.65 (s, 1H), 7.01 (d, J=8.59 Hz, 2H), 7.28-7.35 (m, 2H). M.S. (calcd): 489.2 (MH⁺), M.S. (found): 489.2 (ESI) (MH⁺).

### Example 115

### 2-(4-acetylpiperazin-1-yl)-4-{[(1R)-1-(4-ethoxyphenyl)ethyl]amino}-6-isopropyl-5,6-dihydro-7H-pyrrolo[3,4-d]pyrimidin-7-one

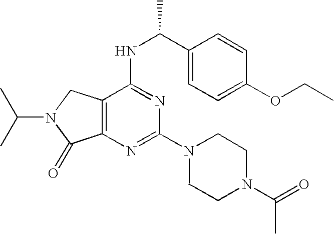

Following a procedure similar to that described in General Procedure 1 and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (156 mg, 55%) was obtained as its TFA salt. HPLC: k′ 11.85; Purity: >97% (215 nm), >97% (254 nm), >96% (280 nm); Rₜ: 1.67 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.24-1.37 (m, 9H), 1.57 (d, J=7.03 Hz, 3H), 2.11 (s, 3H), 3.52-3.87 (m, 8H), 3.98 (q, J=7.03 Hz, 2H), 4.33 (s, 2H), 4.40-4.51 (m, 1H), 5.28 (q, J=7.03 Hz, 1H), 6.85 (d, J=8.59 Hz, 2H), 7.28 (d, J=8.59 Hz, 2H). M.S. (calcd): 467.3 (MH⁺), M.S. (found): 467.3 (ESI) (MH⁺)

### Example 116

### 2-(4-acetylpiperazin-1-yl)-4-[(2R)-2-benzylpyrrolidin-1-yl]-6-isopropyl-5,6-dihydro-7H-pyrrolo[3,4-d]pyrimidin-7-one

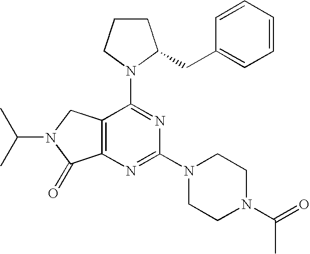

Following a procedure similar to that described in General Procedure 1, starting from (2R)-2-benzylpyrrolidine and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (195 mg, 69%) was obtained as its TFA salt. HPLC: k′ 12.68; Purity: >99% (215 nm), >99% (254 nm), >99% (280 nm); Rₜ: 1.78 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.27-1.38 (m, 6H), 1.90 (s, 2H), 2.00-2.11 (m, 2H), 2.14 (s, 3H), 2.70-2.83 (m, 1H), 3.22 (d, J=8.98 Hz, 1H), 3.65-3.76 (m, 4H), 3.78-3.98 (m, 6H), 4.39-4.55 (m, 1H), 4.71 (s, 2H), 4.79 (s, 1H), 7.21 (d, J=6.25 Hz, 3H), 7.25-7.33 (m, 2H). M.S. (calcd): 463.3 (MH⁺), M.S. (found): 463.3 (ESI) (MH⁺).

### Example 117

### 2-(4-acetylpiperazin-1-yl)-4-[(2S)-2-benzylpyrrolidin-1-yl]-6-isopropyl-5,6-dihydro-7H-pyrrolo[3,4-d]pyrimidin-7-one

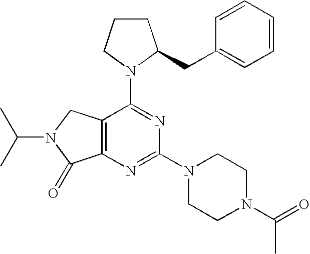

Following a procedure similar to that described in General Procedure 1, starting from (2S)-2-benzylpyrrolidine and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (235 mg, 84%) was obtained as its TFA salt. HPLC: k′ 12.78; Purity: >96% (215 nm), >96% (254 nm), >96% (280 nm); Rₜ: 1.79 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.33 (m, 6H), 1.85-1.95 (m, 2H), 2.02-2.12 (m, 2H), 2.14 (s, 3H), 2.69-2.81 (m, 1H), 3.18-3.26 (m, 1H), 3.67-3.76 (m, 4H), 3.79-4.00 (m, 6H), 4.41-4.57 (m, 1H), 4.72 (s, 2H), 4.81 (s, 1H), 7.22 (d, J=6.64 Hz, 3H), 7.24-7.35 (m, 2H). M.S. (calcd): 463.3 (MH⁺), M.S. (found): 463.3 (ESI) (MH⁺).

### Example 118

### 2-(4-acetylpiperazin-1-yl)-6-ethyl-4-{[2-(4-fluorophenyl)-1,1-dimethylethyl]amino}-5,6-dihydro-7H-pyrrolo[3,4-d]pyrimidin-7-one

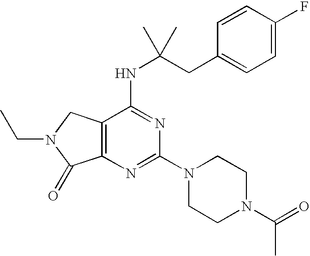

Following a procedure similar to that described in General Procedure 1 and after purification by silica gel chromatography (gradient 100% ethyl acetate to 40% methanol: ethyl acetate) followed by preparative LCMS (gradient 35-55% CH₃CN in H₂O containing 0.1% trifluoroacetic acid), the title compound (25 mg, 20%) was obtained as its TFA salt. HPLC: k′ 12.00; Purity: >99% (215 nm), >99% (254 nm), >99% (280 nm); Rₜ: 1.69 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.23 (t, J=7.23 Hz, 3H), 1.47 (s, 6H), 2.13 (s, 3H), 3.28 (s, 2H), 3.60 (q, J=7.42 Hz, 2H), 3.66-3.77 (m, 4H), 3.84-3.91 (m, 2H), 3.92-4.00 (m, 2H), 4.20 (s, 2H), 6.94 (t, J=8.79 Hz, 2H), 7.01-7.09 (m, 2H). M.S. (calcd): 455.3 (MH⁺), M.S. (found): 455.3 (ESI) (MH⁺).

### Example 119

### 2-(4-ethylpiperazin-1-yl)-6-isopropyl-4-{[phenyl(pyridin-2-yl)methyl]amino}-5,6-dihydro-7H-pyrrolo[3,4-d]pyrimidin-7-one

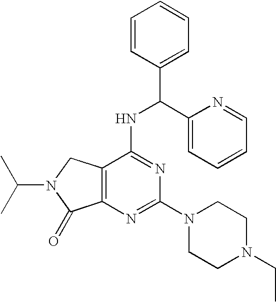

Following a procedure similar to that described in General Procedure 1 and after purification by reverse phase HPLC (gradient 25-45% CH₃CN in H₂O containing 0.1% trifluoroacetic acid) followed by lyophilization from CH₃CN/H₂O, the title compound (6 mg, 32%) was obtained as its TFA salt. HPLC: k′ 8.92; Purity: >99% (215 nm), >99% (254 nm), >99% (280 nm); Rₜ: 1.28 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29-1.37 (m, 9H), 2.73-2.91 (m, 2H), 3.05-3.20 (m, 4H), 3.41-3.55 (m, 2H), 4.34 (s, 2H), 4.53 (q, J=6.64 Hz, 1H), 4.66-4.78 (m, 2H), 6.52 (s, 1H), 7.32-7.43 (m, 5H), 7.51-7.59 (m, 1H), 7.68-7.75 (m, 1H), 8.05-8.12 (m, 1H), 8.64 (d, J=5.47 Hz, 1H). M.S. (calcd): 472.282 (MH⁺), M.S. (found): 472.2 (MH⁺). Found: C, 46.78; H, 4.55; N, 11.64. C₂₇H₃₃N₇O×3.5C₂HF₃O₂ has C, 46.90; H, 4.23; N, 11.26%.

### Example 120

### 2-(4-ethylpiperazin-1-yl)-4-[(9-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one

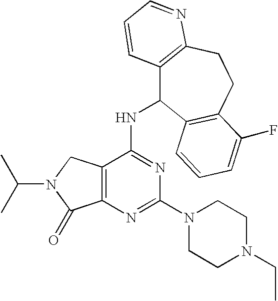

Following a procedure similar to that described in General Procedure 1, starting from 9-fluoro-10,11-dihydro-5H-benzo[4,5]cyclohepta[1,2-b]pyridin-5-amine (for the preparation see: Cheng, Yun-Xing; Luo, Xuehong; Tomaszewski, Miroslaw; Walpole, Chistopher PCT Int. Appl. (2003), WO2003051276A2) and after purification by reverse phase HPLC (gradient 25-45% CH₃CN in H₂O containing 0.1% trifluoroacetic acid) followed by lyophilization from CH₃CN/H₂O, the title compound (30 mg, 16%) was obtained as its TFA salt. HPLC: k′ 7.35; Purity: >94.6% (215 nm), >93% (254 nm), >93% (280 nm); Rₜ: 1.08 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.64 Hz, 6H), 1.38 (t, J=7.42 Hz, 3H), 2.90-3.10 (m, 2H), 3.19-3.35 (m, 4H), 3.38-3.48 (m, 2H), 3.25-3.64 (m, 3H), 3.75-3.85 (m, 1H), 4.31 (s, 2H), 4.52 (q, J=6.64 Hz, 1H), 4.89-4.99 (m, 2H), 6.77 (s, 1H), 7.12 (t, J=9.37 Hz, 1H), 7.26-7.31 (m, 1H), 7.41 (d, J=7.42 Hz, 1H), 7.72-7.80 (m, 1H), 8.57-8.63 (m, 2H). M.S. (calcd): 516.288 (MH⁺), M.S. (found): 516.3 (MH⁺). Found: C, 48.49; H, 4.44; N, 11.11. C₂₉H₃₄FN₇O×3.1C₂HF₃O₂ has C, 48.65; H, 4.30; N, 11.28%.

### Example 121

### 2-[[2-(dimethylamino)ethyl](methyl)amino]-4-[(9-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one

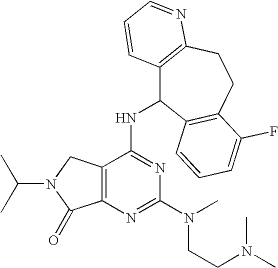

Following a procedure similar to that described in General Procedure 1, starting from 9-fluoro-10,11-dihydro-5H-benzo[4,5]cyclohepta[1,2-b]pyridin-5-amine (for the preparation see: Cheng, Yun-Xing; Luo, Xuehong; Tomaszewski, Miroslaw; Walpole, Chistopher PCT Int. Appl. (2003), WO2003051276A2) and after purification by reverse phase HPLC (gradient 45-65% CH₃CN in H₂O containing 0.1% trifluoroacetic acid) followed by lyophilization from CH₃CN/H₂O, the title compound (52 mg, 14%) was obtained as its TFA salt. HPLC: k′ 15.46; Purity: >99% (215 nm), >99% (254 nm), >99% (280 nm); Rₜ: 1.19 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=7.03 Hz, 6H), 2.92-2.99 (m, 7H), 3.19-3.22 (m, 2H), 3.35 (t, J=5.47 Hz, 3H), 3.45-3.52 (m, 1H), 3.57-3.64 (m, 1H), 3.80-3.93 (m, 3H), 4.33 (s, 2H), 4.47-4.54 (m, 1H), 6.84 (s, 1H), 7.13 (dt, J=1.18, 8.60 Hz, 1H), 7.28 (m, 1H), 7.42 (d, J=7.81 Hz, 1H), 7.87 (t, J=8.20 Hz, 1H), 8.66 (dd, J=1.17, 5.86 Hz, 1H), 8.76 (dd, J=1.18, 8.21 Hz, 1H). M.S. (calcd): 504.288 (MH⁺), M.S. (found): 504.2 (MH⁺). Found: C, 47.17; H, 3.64; N, 11.16. C₂₈H₃₄FN₇O×3.3C₂HF₃O₂ has C, 47.23; H, 4.27; N, 11.14%.

### Example 122

### 2-(4-acetylpiperazin-1-yl)-6-isopropyl-4-{[2-methyl-4-(trifluoromethoxy)benzyl]amino}-5,6-dihydro-7H-pyrrolo[3,4-d]pyrimidin-7-one

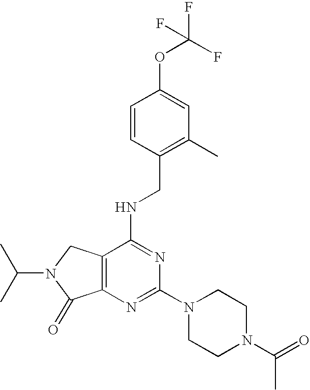

Following a procedure similar to that described in General Procedure 1 and after purification by preparative LCMS (gradient 35-55% CH₃CN in H₂O containing 0.1% trifluoroacetic acid) and lyophilized from CH₃CN/H₂O, the title compound (48 mg, 38%) was obtained as its TFA salt. HPLC: k′ 13.94; Purity: >98% (215 nm), >97% (254 nm), >97% (280 nm); Rₜ: 1.94 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (s, 3H), 1.31 (s, 3H), 2.12 (s, 3H), 2.41 (s, 3H), 3.56-3.64 (m, 4H), 3.79 (dd, J=6.25, 3.91 Hz, 2H), 3.86 (dd, J=6.25, 4.30 Hz, 2H), 4.26 (s, 2H), 4.45-4.55 (m, 1H), 4.73 (s, 2H), 7.04-7.09 (m, 1H), 7.10-7.14 (m, 1H), 7.38 (d, J=8.20 Hz, 1H). M.S. (calcd): 507.2 (MH⁺), M.S. (found): 507.2 (ESI) (MH⁺). Found: C, 50.99; H, 4.8; N, 14.02. C₂₄H₂₉N₆O₃F₃×0.9 CF₃CO₂H has C, 50.87; H, 4.95; N, 13.80%.

### Example 123

### 2-(4-acetylpiperazin-1-yl)-4-[2-(4-chlorophenyl)-2-methylmorpholin-4-yl]-6-isopropyl-5,6-dihydro-7H-pyrrolo[3,4-d]pyrimidin-7-one

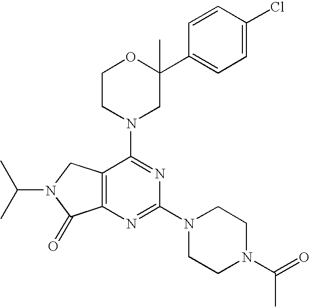

Following a procedure similar to that described in General Procedure 1 and after purification by silica gel chromatography (gradient 5-30% methanol:ethyl acetate) followed by preparative LCMS (gradient 35-55% CH₃CN in H₂O containing 0.1% trifluoroacetic acid) and lyophilized from CH₃CN/H₂O, the title compound (37 mg, 29%) was obtained as its TFA salt. HPLC: k′ 14.46; Purity: >99% (215 nm), >99% (254 nm), >99% (280 nm); Rₜ: 2.01 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (dd, J=8.40, 6.84 Hz, 6H), 1.49 (s, 3H), 2.16 (s, 3H), 3.56 (d, J=14.06 Hz, 1H), 3.62-3.76 (m, 7H), 3.85-3.92 (m, 3H), 3.92-3.98 (m, 2H), 4.44-4.59 (m, 3H), 4.82-4.89 (m, 1H), 7.30-7.35 (m, 2H), 7.46-7.51 (m, 2H). M.S. (calcd): 513.2 (MH⁺), M.S. (found): 513.3 (ESI) (MH⁺). Found: C, 53.69; H, 5.18; N, 13.82. C₂₆H₃₃N₆O₃Cl×1.0CF₃CO₂H has C, 53.63; H, 5.47; N, 13.40%.

### Example 124

### 4-{[2-(4-chlorophenyl)-1,1-dimethylethyl]amino}-2-(4-isobutyrylpiperazin-1-yl)-6-isopropyl-5,6-dihydro-7H-pyrrolo[3,4-d]pyrimidin-7-one

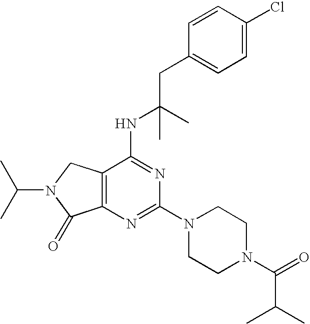

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (100% ethyl acetate) followed by preparative LCMS (gradient 45-65% CH₃CN in H₂O containing 0.1% trifluoroacetic acid) and lyophilized from CH₃CN/H₂O, the title compound (45 mg, 57%) was obtained as its TFA salt. HPLC: k′ 16.23; Purity: >99% (215 nm), >99% (254 nm), >99% (280 nm); Rₜ: 2.24 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.11 (s, 3H), 1.13 (s, 3H), 1.28 (s, 3H), 1.30 (s, 3H), 1.49 (s, 6H), 2.96-3.04 (m, 1H), 3.29-3.32 (m, 2H), 3.71-3.81 (m, 4H), 3.86-3.92 (m, 2H), 3.92-3.98 (m, 2H), 4.17 (s, 2H), 4.45-4.54 (m, 1H), 7.03-7.08 (m, 2H), 7.21-7.25 (m, 2H). M.S. (calcd): 513.3 (MH⁺), M.S. (found): 513.3 (ESI) (MH⁺). Found: C, 54.00; H, 5.99; N, 12.95. C₂₇H₃₇N₆O₂Cl×1.2CF₃CO₂H×0.2H₂O has C, 54.03; H, 5.95; N, 12.86%.

### Example 125

### 4-{[2-(4-chlorophenyl)-1,1-dimethylethyl]amino}-2-[4-(2,2-dimethylpropanoyl)piperazin-1-yl]-6-isopropyl-5,6-dihydro-7H-pyrrolo[3,4-d]pyrimidin-7-one

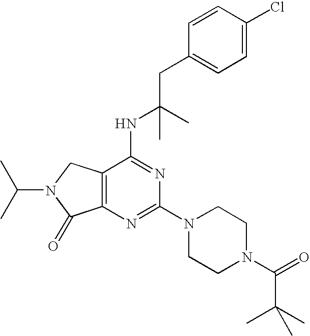

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (100% ethyl acetate) followed by preparative LCMS (gradient 45-65% CH₃CN in H₂O containing 0.1% trifluoroacetic acid) and lyophilized from CH₃CN/H₂O, the title compound (46 mg, 56%) was obtained as its TFA salt. HPLC: k′ 17.54; Purity: >99% (215 nm), >99% (254 nm), >99% (280 nm); Rₜ: 2.41 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.31 (s, 9H), 1.50 (s, 6H), 3.29-3.32 (m, 2H), 3.81-3.87 (m, 4H), 3.88-3.93 (m, 4H), 4.19 (s, 2H), 4.44-4.53 (m, 1H), 7.04-7.08 (m, 2H), 7.21-7.26 (m, 2H). M.S. (calcd): 527.3 (MH⁺), M.S. (found): 527.2 (ESI) (MH⁺). Found: C, 55.83; H, 6.17; N, 12.88. C₂₈H₃₉N₆O₂Cl×1.0CF₃CO₂H×0.2H₂O has C, 55.89; H, 6.32; N, 13.03%.

### Example 126

### 4-{[2-(4-chlorophenyl)-1,1-dimethylethyl]amino}-2-[4-(cyclopropylcarbonyl)piperazin-1-yl]-6-isopropyl-5,6-dihydro-7H-pyrrolo[3,4-d]pyrimidin-7-one

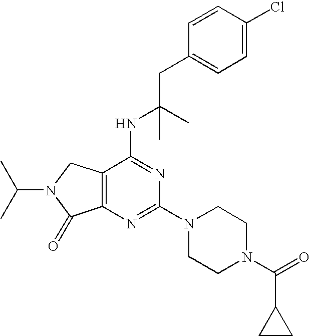

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (100% ethyl acetate) followed by preparative LCMS (gradient 45-65% CH₃CN in H₂O containing 0.1% trifluoroacetic acid) and lyophilized from CH₃CN/H₂O, the title compound (43 mg, 56%) was obtained as its TFA salt. HPLC: k′ 15.65; Purity: >99% (215 nm), >99% (254 nm), >99% (280 nm); Rₜ: 2.17 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 0.82-0.88 (m, 2H), 0.88-0.94 (m, 2H), 1.28 (s, 3H), 1.30 (s, 3H), 1.49 (s, 6H), 1.97-2.05 (m, 1H), 3.69-4.07 (m, 10H), 4.19 (s, 2H), 4.44-4.54 (m, 1H), 7.04-7.10 (m, 2H), 7.21-7.26 (m, 2H). M.S. (calcd): 511.3 (MH⁺), M.S. (found): 511.2 (ESI) (MH⁺). Found: C, 52.47; H, 5.50; N, 12.64. C₂₇H₃₅N₆O₂Cl×1.3CF₃CO₂H×1.0H₂O has C, 52.49; H, 5.70; N, 12.41%.

### Example 127

### 4-(4-{[2-(4-chlorophenyl)-1,1-dimethylethyl]amino}-6-isopropyl-7-oxo-6,7-dihydro-5H-pyrrolo[3,4-d]pyrimidin-2-yl)-N,N-dimethylpiperazine-1-carboxamide

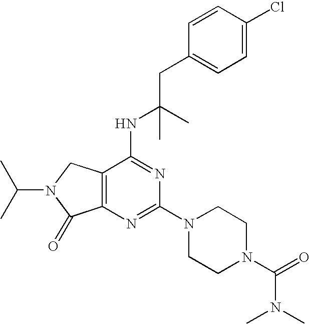

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol: ethyl acetate) followed by preparative LCMS (gradient 45-65% CH₃CN in H₂O containing 0.1% trifluoroacetic acid and then with gradient 55-75% CH₃CN in H₂O containing 10 mM NH₄HCO₃), the product was dissolved in acetonitrile containing 0.1% TFA and stirred for one h and lyophilized from CH₃CN/H₂O to give the title compound (29 mg, 38%) as its TFA salt. HPLC: k′ 15.31; Purity: >99% (215 nm), >99% (254 nm), >99% (280 nm); Rₜ: 2.12 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.49 (s, 6H), 2.89 (s, 6H), 3.26-3.36 (m, 2H), 3.38-3.43 (m, 4H), 3.89-3.95 (m, 4H), 4.20 (s, 2H), 4.44-4.53 (m, 1H), 7.04-7.09 (m, 2H), 7.21-7.27 (m, 2H). M.S. (calcd): 514.3 (MH⁺), M.S. (found): 514.2 (ESI) (MH⁺). Found: C, 51.14; H, 5.70; N, 14.64. C₂₆H₃₆N₇O₂Cl×1.3CF₃CO₂H×0.5H₂O has C, 51.17; H, 5.75; N, 14.61%.

### Example 128

### 4-(4-{[2-(4-chlorophenyl)-1,1-dimethylethyl]amino}-6-isopropyl-7-oxo-6,7-dihydro-5H-pyrrolo[3,4-d]pyrimidin-2-yl)piperazine-1-carbaldehyde

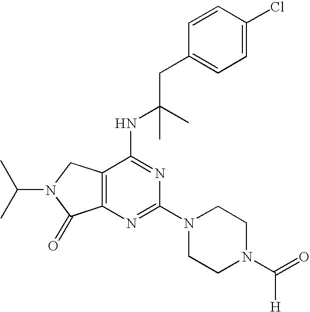

Following a procedure similar to that described in General Procedure 2, starting from 2-chloro-4-{[2-(4-chlorophenyl)-1,1-dimethylethyl]amino}-6-isopropyl-5,6-dihydro-7H-pyrrolo[3,4-d]pyrimidin-7-one (Intermediate 83) and after purification by silica gel chromatography (gradient 5-30% methanol: ethyl acetate) followed by preparative LCMS (gradient 45-65% CH₃CN in H₂O containing 0.1% trifluoroacetic acid) and lyophilized from CH₃CN/H₂O, the title compound (8.8 mg, 12%) was obtained as its TFA salt. HPLC: k′ 14.08; Purity: >99% (215 nm), >99% (254 nm), >99% (280 nm); Rₜ: 1.96 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (s, 3H), 1.30 (s, 3H), 1.48 (s, 6H), 3.59 (dd, J=6.25, 4.30 Hz, 2H), 3.62-3.67 (m, 2H), 3.89-3.93 (m, 2H), 3.93-3.98 (m, 2H), 4.16 (s, 2H), 4.45-4.55 (m, 1H), 4.86-4.91 (m, 2H), 7.02-7.07 (m, 2H), 7.20-7.25 (m, 2H), 8.13 (s, 1H). M.S. (calcd): 471.2 (MH⁺), M.S. (found): 471.3 (ESI) (MH⁺). Found: C, 50.72; H, 5.12; N, 13.38. C₂₄H₃₁N₆O₂Cl×1.4CF₃CO₂H×0.2H₂O has C, 50.75; H, 5.21; N, 13.25%.

### Example 129

### 2-(4-Acetylpiperazin-1-yl)-4-[2-(4-fluorobenzyl)-pyrrolidin-1-yl]-6-isopropyl-5H-pyrrolo[3,4-d]pyrimidin-7(6H)-one

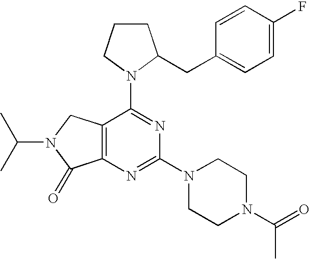

Following a procedure similar to that described in General Procedure 4, starting from 2-chloro-4-[2-(4-fluorobenzyl)-pyrrolidin-1-yl]-6-isopropyl-5H-pyrrolo[3,4-d]pyrimidin-7(6H)-one (Intermediate 107) and after purification by silica gel chromatography (5% MeOH in CH₂Cl₂), then crystallization from EtOAc/Hexanes, the title compound was obtained as a solid (0.2 g, 75%). HPLC: 97.2%. M.S. (calcd): 480.6 (MH⁺), M.S. (found): 481.32 (ESI) (MH⁺). Found: C, 64.32; H, 7.17; N, 17.46. C₂₆H₃₃FN₆O₂×0.25H₂O has C, 64.38; H, 6.96; N, 17.32.

### Example 130

### 2-(4-Acetylpiperazin-1-yl)-4-[1-(4-fluorophenyl)-cyclopropylamino]-6-isopropyl-5H-pyrrolo[3,4-d]pyrimidin-7(6H)-one

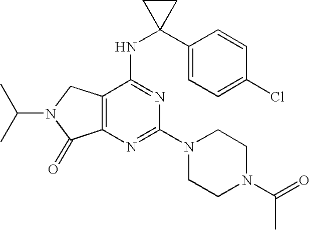

Following a procedure similar to that described in General Procedure 4, starting from 2-chloro-4-[1-(4-fluorophenyl)-cyclopropylamino]-6-isopropyl-5H-pyrrolo[3,4-d]pyrimidin-7(6H)-one (Intermediate 110) and after purification by silica gel chromatography (5% MeOH in CH₂Cl₂), then crystallization from EtOAc/Hexanes, the title compound was obtained as a solid (0.185 g, 80%). HPLC: 98.0%. ¹H NMR (400 MHz, CDCl₃) δ ppm 1.17 (d, J=5.85 Hz, 6H), 1.34-1.45 (m, 4H), 2.13 (s, 3H), 3.43 (s, 2H), 3.61 (s, 2H), 3.78 (s, 2H), 3.86 (s, 2H), 3.97 (s, 2H), 4.55-4.66 (m, 1H), 5.52-5.65 (m, 1H), 7.11 (d, J=8.20 Hz, 2H), 7.22-7.29 (m, 2H); M.S. (calcd): 469.0 (MH⁺), M.S. (found): 469.13 and 470.91 (ESI) (MH⁺). Found: C, 60.69; H, 6.41; N, 17.75. C₂₄H₂₉ClN₆O₂×⅓H₂O has C, 60.69; H, 6.30; N, 17.69.

### Example 131

### 2-(4-Acetylpiperazin-1-yl)-6-isopropyl-4-(2-(3-methoxyphenyl)pyrrolidin-1-yl)-5H-pyrrolo[3,4-d]pyrimidin-7(6H)-one

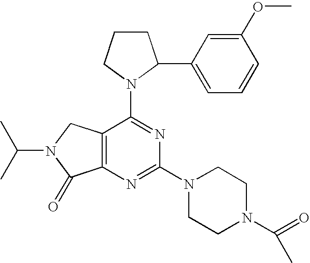

Following a procedure similar to that described in General Procedure 4, starting from 2-chloro-6-isopropyl-4-(2-(3-methoxyphenyl)pyrrolidin-1-yl)-5H-pyrrolo[3,4-d]pyrimidin-7(6H)-one (Intermediate 111) and after purification by silica gel chromatography (3% MeOH in CH₂Cl₂), the title compound was obtained as a solid (0.115 g, 62%). HPLC: 98.19%. ¹H NMR (400 MHz, CDCl₃) δ ppm 1.11 (s, 3H), 1.21 (s, 3H), 1.94-2.07 (m, 3H), 2.11 (s, 3H), 2.31-2.43 (m, 1H), 3.22-3.92 (m, 10H), 3.78 (s, 3H), 3.96-4.06 (m, 1H), 4.33 (s, 1H), 4.60 (s, 1H), 5.18 (s, 1H), 6.67 (s, 1H), 6.75 (d, J=7.04 Hz, 2H), 7.20-7.25 (m, 1H); M.S. (calcd): 478.6 (MH⁺), M.S. (found): 479.30 (ESI) (MH⁺). Found: C, 64.24; H, 7.24; N, 17.06. C₂₆H₃₄N₆O₃×0.3H₂O has C, 64.44; H, 7.21; N, 17.34.

### Example 132

### 2-(4-Acetylpiperazin-1-yl)-4-(2-(3-chlorophenyl)pyrrolidin-1-yl)-6-isopropyl-5H-pyrrolo[3,4-d]pyrimidin-7(6H)-one

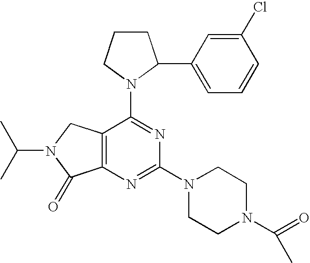

Following a procedure similar to that described in General Procedure 4, starting from 2-chloro-4-(2-(3-chlorophenyl)pyrrolidin-1-yl)-6-isopropyl-5H-pyrrolo[3,4-d]pyrimidin-7(6H)-one (Intermediate 112) and after purification by silica gel chromatography (3% MeOH in CH₂Cl₂), the title compound was obtained as a solid (75 mg, 48%). HPLC: 96.73%. ¹H NMR (400 MHz, CDCl₃) δ ppm 1.19 (s, 3H), 1.25 (d, J=5.87 Hz, 3H), 1.96 (d, J=11.35 Hz, 1H), 2.07 (s, 2H), 2.10 (s, 3H), 2.33-2.46 (m, 1H), 3.23-3.90 (m, 10H), 3.98-4.06 (m, 1H), 4.39 (d, J=12.91 Hz, 1H), 4.60-4.71 (m, 1H), 5.20 (d, J=7.83 Hz, 1H), 7.05 (dd, J=7.43, 1.57 Hz, 1H), 7.14 (d, J=1.57 Hz, 1H), 7.18-7.25 (m, 2H); M.S. (calcd): 483.0 (MH⁺), M.S. (found): 483.09 and 485.04 (ESI) (MH⁺). Found: C, 61.61; H, 6.87; N, 16.85. C₂₅H₃₁ClN₆O₂×0.25H₂O has C, 61.59; H, 6.51; N, 17.24.

### Example 133

### 2-(4-Acetylpiperazin-1-yl)-4-(5-chloro-2,3-dihydro-1H-inden-1-ylamino)-6-isopropyl-5H-pyrrolo[3,4-d]pyrimidin-7(6H)-one

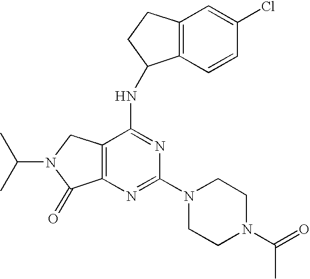

Following a procedure similar to that described in General Procedure 4, starting from 2-chloro-4-(5-chloro-2,3-dihydro-1H-inden-1-ylamino)-6-isopropyl-5H-pyrrolo[3,4-d]pyrimidin-7(6H)-one (Intermediate 108) and after purification by silica gel chromatography (CH₂Cl₂: MeOH 30:1), the title compound was obtained as a solid (95 mg, 93%). HPLC purity>95%, Rₜ: 10.5 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 1.15-1.33 (m, 6H), 1.89-2.05 (m, 1H), 2.09-2.20 (m, 3H), 2.59-2.78 (m, 1H), 2.85-3.08 (m, 2H), 3.42-3.57 (m, 2H), 3.59-3.76 (m, 2H), 3.77-4.01 (m, 4H), 4.07 (s, 2H), 4.67 (ddd, J=13.46, 7.02, 6.83 Hz, 1H), 4.77 (d, J=7.80 Hz, 1H), 5.61-5.82 (m, 1H), 7.10-7.26 (m, 3H). M.S. (calcd): 468.99 (MH⁺), M.S. (found): 469.3 (ESI) (MH⁺).

### Example 134

### 4-(1-(4-Fluorophenyl)-2-methylpropan-2-ylamino)-6-isopropyl-2-(6-oxohexahydro[1,2-a]pyrazin-2(1H)-yl)-5H-pyrrolo[3,4-d]pyrimidin-7(6H)-one

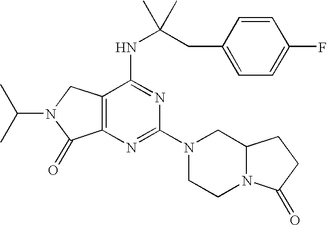

Following a procedure similar to that described in General Procedure 4, starting from 2-chloro-4-[2-(4-fluoro-phenyl)-1,1-dimethyl-ethylamino]-6-isopropyl-5,6-dihydro-pyrrolo[3,4-d]pyrimidin-7-one (Intermediate 114) and after purification by silica gel chromatography (CH₂Cl₂:MeOH 30:1), the title compound was obtained as a solid (95 mg, 93%). HPLC purity>98%, Rₜ: 10.6 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 1.25 (d, J=6.63 Hz, 6H), 1.47 (d, J=4.68 Hz, 6H), 1.62-1.81 (m, 1H), 2.13-2.32 (m, 1H), 2.38-2.52 (m, 2H), 2.55-2.77 (m, 1H), 2.81-3.01 (m, 2H), 3.12-3.34 (m, 2H), 3.53-3.73 (m, 1H), 3.92 (s, 2H), 4.03 (s, 1H), 4.11 (d, J=10.53 Hz, 1H), 4.68 (dt, J=13.55, 6.68 Hz, 1H), 4.93 (d, J=9.36 Hz, 1H), 5.17 (d, J=13.26 Hz, 1H), 6.84-7.05 (m, 4H). M.S. (calcd): 481.59 (MH⁺), M.S. (found): 481.46 (ESI) (MH⁺).

### Example 135

### 6-Isopropyl-2-(6-oxohexahydropyrrolo[1,2-a]pyrazin-2(1H)-yl)-4-(quinolin-3-ylmethylamino)-5H-pyrrolo[3,4-d]pyrimidin-7(6M)-one

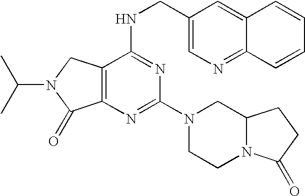

Following a procedure similar to that described in General Procedure 4, starting from 2-Chloro-6-isopropyl-4-(quinolin-3-ylmethylamino)-5H-pyrrolo[3,4-d]pyrimidin-7(6H)-one (Intermediate 109) and after purification by silica gel chromatography (CH₂Cl₂: MeOH 20:1), the title compound was obtained as a solid (84 mg, 65%). HPLC purity>97%, Rₜ: 9.0 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 1.25 (d, J=6.63 Hz, 6H), 1.48-1.69 (m, 1H), 2.10 (dd, J=13.27, 5.46 Hz, 1H), 2.33-2.45 (m, 2H), 2.52 (dd, J=12.88, 10.93 Hz, 1H), 2.66-2.86 (m, 2H), 3.47 (d, J=6.63 Hz, 1H), 4.01 (d, J=9.76 Hz, 1H), 4.12 (s, 2H), 4.67 (dt, J=13.37, 6.78 Hz, 1H), 4.80-4.97 (m, 3H), 5.07 (br. s., 1H), 5.27 (dd, J=5.66, 5.66 Hz, 1H), 7.57 (dd, J=7.42, 7.42 Hz, 1H), 7.66-7.86 (m, 2H), 8.02-8.20 (m, 2H), 8.93 (d, J=1.95 Hz, 1H). M.S. (calcd): 470.57 (MH⁺), M.S. (found): 470.64 (ESI) (MH⁺)

### Example 136

### 6-Isopropyl-2-(6-oxohexahydropyrrolo[1,2-a]pyrazin-2(1H)-yl)-4-(quinolin-3-ylmethylamino)-5H-pyrrolo[3,4-d]pyrimidin-7(6M)-one

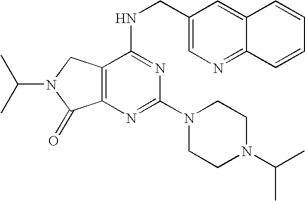

Following a procedure similar to that described in General Procedure 4, starting from 2-Chloro-6-isopropyl-4-(quinolin-3-ylmethylamino)-5H-pyrrolo[3,4-d]pyrimidin-7(6H)-one (Intermediate 109) and after purification by silica gel chromatography (CH₂Cl₂: MeOH 10:1), the title compound was obtained as a solid (92 mg, 73%). HPLC purity>98%, Rₜ: 8.8 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 0.94-1.12 (m, 6H), 1.15-1.35 (m, 6H), 2.49 (br. s., 4H), 2.70 (d, J=6.24 Hz, 1H), 3.88 (br. s., 4H), 4.09 (s, 2H), 4.55-4.77 (m, 1H), 4.91 (d, J=5.46 Hz, 2H), 5.09 (br. s., 1H), 7.56 (dd, J=7.42, 7.42 Hz, 1H), 7.66-7.88 (m, 2H), 8.00-8.23 (m, 2H), 8.93 (d, J=1.56 Hz, 1H). M.S. (calcd): 458.60 (MH⁺), M.S. (found): 458.80 (ESI) (MH⁺).

### Example 137

### 2-(5,6-Dihydro-[1,2,4]triazolo[4,3-a]pyrazin-7(8H)-yl)-6-isopropyl-4-(quinolin-3-ylmethylamino)-5H-pyrrolo[3,4-d]pyrimidin-7(6H)-one

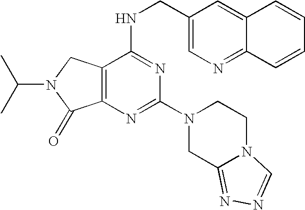

Following a procedure similar to that described in General Procedure 4, starting from 2-Chloro-6-isopropyl-4-(quinolin-3-ylmethylamino)-5H-pyrrolo[3,4-d]pyrimidin-7(6H)-one (Intermediate 109) and after purification by silica gel chromatography (CH₂Cl₂: MeOH 10:1), the title compound was obtained as a solid (116 mg, 94%). HPLC purity>97%, Rₜ: 8.9 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CD₃OD) δ ppm 1.31 (d, J=6.63 Hz, 6H), 4.11 (t, J=4.88 Hz, 2H), 4.18-4.34 (m, 4H), 4.54 (dt, J=13.56, 6.68 Hz, 1H), 4.96 (s, 2H), 5.15 (s, 2H), 7.61 (t, J=7.42 Hz, 1H), 7.75 (t, J=7.42 Hz, 1H), 7.86-8.11 (m, 2H), 8.35 (s, 1H), 8.42 (s, 1H), 8.92 (s, 1H). M.S. (calcd): 454.53 (MH⁺), M.S. (found): 454.58 (ESI) (MH⁺)

### Example 138

### 2-(4-Acetyl-3-methyl-piperazin-1-yl)-4-[2-(4-fluoro-phenyl)-1,1-dimethyl-ethylamino]-6-isopropyl-5,6-dihydro-pyrrolo[3,4-d]pyrimidin-7-one

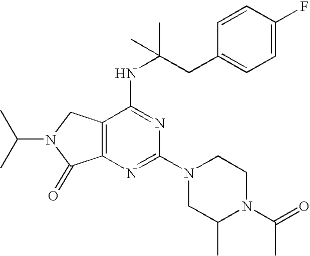

Following a procedure similar to that described in General Procedure 4, starting from 2-chloro-4-[2-(4-fluoro-phenyl)-1,1-dimethyl-ethylamino]-6-isopropyl-5,6-dihydro-pyrrolo[3,4-d]pyrimidin-7-one (Intermediate 114) and after purification by silica gel chromatography (CH₂Cl₂: MeOH), the title compound was obtained as an oil (11 mg, 69%). HPLC purity>99%, Rₜ: 10.6 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (498 MHz, DMSO-d₆) δ ppm 1.01 (d, J=6.07 Hz, 2H), 1.12 (d, J=7.41 Hz, 1H), 1.18 (dd, J=6.74, 1.40 Hz, 6H), 1.37 (d, J=14.09 Hz, 6H), 2.03 (d, J=13.97 Hz, 3H), 2.86 (d, J=8.75 Hz, 1H), 3.21-3.29 (m, 4H), 4.02 (s, 2H), 4.18-4.28 (m, 1H), 4.37 (dt, J=13.42, 6.65 Hz, 1H), 4.53 (d, J=10.57 Hz, 2H), 4.58-4.67 (m, 1H), 7.02-7.11 (m, 4H). M.S. (calcd); 483.61 (MH⁺), M.S. (found); 483.66 (ESI) (MH⁺).

### Example 139

### 2-(4-Acetyl-2-methyl-piperazin-1-yl)-4-[2-(4-fluoro-phenyl)-1,1-dimethyl-ethylamino]-6-isopropyl-5,6-dihydro-pyrrolo[3,4-d]pyrimidin-7-one

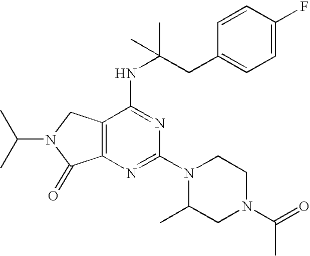

Following a procedure similar to that described in General Procedure 4, starting from 2-chloro-4-[2-(4-fluoro-phenyl)-1,1-dimethyl-ethylamino]-6-isopropyl-5,6-dihydro-pyrrolo[3,4-d]pyrimidin-7-one (Intermediate 114) and after purification by silica gel chromatography (CH₂Cl₂: MeOH), the title compound was obtained as a solid (30 mg, 19%). HPLC purity>86%, Rₜ: 15.0 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 0.86-0.96 (m, 1H), 0.99-1.10 (m, 2H), 1.24 (d, J=6.74 Hz, 6H), 1.37-1.49 (m, 6H), 2.09-2.19 (m, 3H), 2.68-3.08 (m, 1H), 3.19-3.30 (m, 3H), 3.39-3.69 (m, 1H), 3.92 (s, 2H), 4.07 (s, 1H), 4.38-4.46 (m, 1H), 4.57-4.76 (m, 2H), 4.91-5.17 (m, 1H), 6.89-7.02 (m, 4H). M.S. (calcd); 483.61 (MH⁺), M.S. (found); 483.74 (ESI) (MH⁺).

### Example 140

### (R)-7-{4-[2-(4-Fluoro-phenyl)-1,1-dimethyl-ethylamino]-6-isopropyl-7-oxo-6,7-dihydro-5H-pyrrolo[3,4-d]pyrimidin-2-yl}-hexahydro-oxazolo[3,4-a]pyrazin-3-one

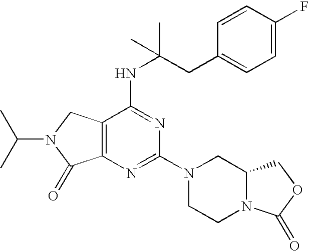

DIPEA (55 μL, 0.30 mmol) was added to a solution of 4-[2-(4-Fluoro-phenyl)-1,1-dimethyl-ethylamino]-2-(3-hydroxymethyl-piperazin-1-yl)-6-isopropyl-5,6-dihydro-pyrrolo[3,4-d]pyrimidin-7-one (Intermediate 118) (69 mg, 0.15 mmol) in CH₂Cl₂ (5 mL) at −5° C. under N₂. A solution of 20% phosgene in toluene (0.03 mL, 0.06 mmol) was added and the solution was stirred for 2 h. DIPEA (20 μL, 0.12 mmol) was added and the solution was stirred at rt for an additional 2 h, then quenched by the addition of water. The organic layer was separated, dried over MgSO₄, filtered and concentrated under reduced pressure to give the title compound as a solid (32 mg, 44%). HPLC purity>99%, Rₜ: 11.6 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 1.25 (d, J=7.03 Hz, 6H), 1.47 (d, J=5.86 Hz, 6H), 2.84 (dd, J=12.88, 10.83 Hz, 1H), 2.97-3.07 (m, 2H), 3.15-3.28 (m, 2H), 3.83-3.96 (m, 4H), 3.98-4.06 (m, 1H), 4.18 (s, 1H), 4.46 (t, J=8.64 Hz, 1H), 4.67 (quin, J=6.81 Hz, 1H), 4.90 (d, J=12.00 Hz, 1H), 5.16 (d, J=9.66 Hz, 1H), 6.88-7.01 (m, 4H). M.S. (calcd); 483.56 (MH⁺), M.S. (found); 483.68 (ESI) (MH⁺).

### Example 141

### (R)-2-(Hexahydro-pyrrolo[1,2-a]pyrazin-2-yl)-6-isopropyl-4-[(quinolin-3-ylmethyl)-amino]-5,6-dihydro-pyrrolo[3,4-d]pyrimidin-7-one

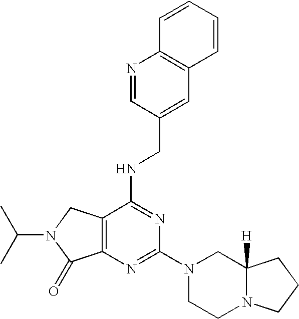

Following a procedure similar to that described in General Procedure 4, starting from 2-Chloro-6-isopropyl-4-(quinolin-3-ylmethylamino)-5H-pyrrolo[3,4-d]pyrimidin-7(6H)-one (Intermediate 109) and after purification by silica gel chromatography (CH₂Cl₂: MeOH), the title compound was obtained as an oil (60 mg, 38%). HPLC purity>98%, Rₜ: 8.8 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 1.16-1.24 (m, 6H), 1.36-1.48 (m, 1H), 1.73-1.82 (m, 2H), 1.83-1.91 (m, 2H), 2.12 (d, J=8.49 Hz, 1H), 2.16 (dd, J=13.32, 2.78 Hz, 1H), 2.58-2.70 (m, 1H), 3.00-3.12 (m, 3H), 4.10 (s, 2H), 4.62 (dt, J=13.47, 6.73 Hz, 1H), 4.79 (d, J=12.00 Hz, 1H), 4.90 (d, J=5.56 Hz, 3H), 5.47 (t, J=5.27 Hz, 1H), 7.54 (t, J=7.47 Hz, 1H), 7.68-7.80 (m, 2H), 8.11 (d, J=1.76 Hz, 2H), 8.92 (d, J=2.05 Hz, 1H). M.S. (calcd); 458.58 (MH⁺), M.S. (found); 458.73 (ESI) (MH⁺).

### Example 142

### 2-(4-Acetyl-3-methyl-piperazin-1-yl)-6-isopropyl-4-[(quinolin-3-ylmethyl)amino]-5,6-dihydro-pyrrolo[3,4-d]pyrimidin-7-one

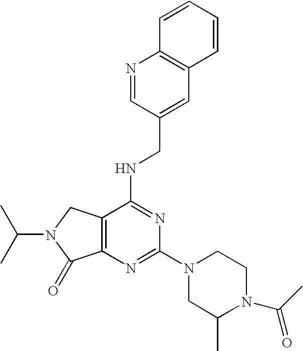

Following a procedure similar to that described in General Procedure 4, starting from 2-Chloro-6-isopropyl-4-(quinolin-3-ylmethylamino)-5H-pyrrolo[3,4-d]pyrimidin-7(6H)-one (Intermediate 109) and after purification by silica gel column chromatography (CH₂Cl₂: MeOH), the title compound was obtained as an oil (65 mg, 42%). HPLC purity>97%, Rₜ: 9.0 minutes, Conditions: Column: ACE C₁₈, 5 μm, 4 60×150 mm; Gradient: 05-45% B in 20 min, 25° C. Solvents: A: 0.1% H₃PO₄ in water, B: MeCN. ¹H NMR (400 MHz, CDCl₃) δ ppm 1.03 (m, 3H), 1.25 (d, J=6.73 Hz, 6H), 2.10 (d, J=12.00 Hz, 3H), 2.95 (d, J=9.37 Hz, 2H), 3.07-3.20 (m, 1H), 3.30-3.59 (m, 1H), 4.11 (s, 2H), 4.38-4.82 (m, 4H), 4.84-4.97 (m, 2 H), 5.18 (t, J=5.71 Hz, 1H), 7.53-7.60 (m, 1H), 7.69-7.81 (m, 2H), 8.06-8.13 (m, 2H), 8.93 (d, J=2.05 Hz, 1H). M.S. (calcd); 474.58 (MH⁺), M.S. (found); 474.61 (ESI) (MH⁺).

### Example 143

### 4-(4-acetylpiperazin-1-yl)-8-propan-2-yl-2-[(5-tert-butyl-1H-pyrazol-3-yl)methylamino]-3,5,8-triazabicyclo[4.3.0]nona-1,3,5-trien-7-one

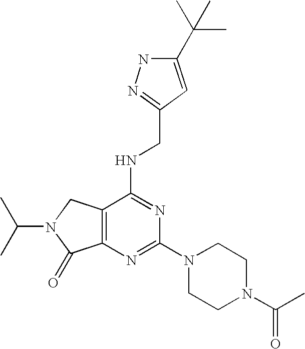

Following General Procedure 1, the title compound was obtained as a solid (61.0 mg, 44.0%) following purification by preparative LCMS (gradient 35-55% CH₃CN in H₂O containing 10 mM NH₄HCO₃) and lyophilization from CH₃CN/H₂O. HPLC purity: >98% (215 nm), >98% (254 nm), >98% (280 nm); Rₜ: 1.28 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.23-1.36 (m, 15H), 2.13 (s, 3H), 3.50-3.63 (m, 4H), 3.75-3.92 (m, 4H), 4.21 (s, 2H), 4.46-4.57 (m, 1H), 4.62 (s, 2H), 6.04 (s, 1H). MS [M+H]⁺ 455.3 (ESI). HRMS m/z calcd for C₂₃H₃₅N₈O₂ [M+H]⁺ 455.2877, found 455.2880.

### Example 144

### 4-(4-acetylpiperazin-1-yl)-2-[(5-phenyl-1,2-oxazol-3-yl)methylamino]-8-propan-2-yl-3,5,8-triazabicyclo[4.3.0]nona-1,3,5-trien-7-one

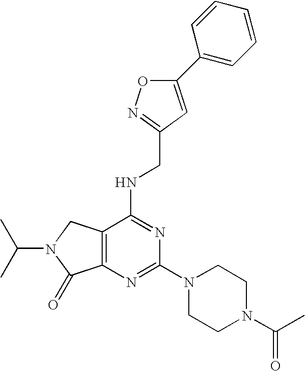

Following a procedure similar to that described in General Procedure 1, the title compound was obtained as a solid (38.0 mg, 26.2%) following purification by preparative LCMS (gradient 35-55% CH₃CN in H₂O containing 10 mM NH₄HCO₃) and lyophilization from CH₃CN/H₂O₀ HPLC purity: >99% (215 nm), >99% (254 nm), >99% (280 nm); Rₜ: 1.56 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=6.64 Hz, 6H), 2.08 (s, 3H), 3.50 (dd, J=6.25, 4.30 Hz, 2H), 3.53-3.58 (m, 2H), 3.77-3.83 (m, 2H), 3.83-3.88 (m, 2H), 4.25 (s, 2H), 4.48-4.57 (m, 1H), 4.76 (s, 2H), 6.75 (s, 1H), 7.41-7.51 (m, 3H), 7.76-7.82 (m, 2H). MS [M+H]⁺ 476.2 (ESI). HRMS m/z calcd for C₂₅H₃₀N₇O₃ [M+H]⁺ 476.2404, found 476.2406.

### Example 145

### 4-(4-acetylpiperazin-1-yl)-2-[(3-phenyl-1,2,4-oxadiazol-5-yl)methylamino]-8-propan-2-yl-3,5,8-triazabicyclo[4.3.0]nona-1,3,5-trien-7-one

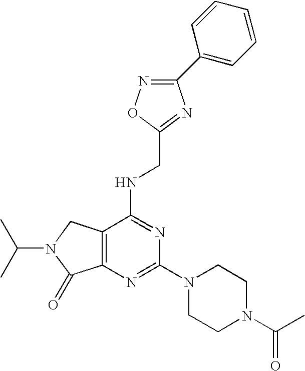

Following a procedure similar to that described in General Procedure 1, the title compound was obtained as a solid (45.0 mg, 27.7%) following purification by preparative LCMS (gradient 35-55% CH₃CN in H₂O containing 10 mM NH₄HCO₃) and lyophilization from CH₃CN/H₂O. HPLC purity: >96% (215 nm), >99% (254 nm), >99% (280 nm); Rₜ: 1.53 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.33 (d, J=6.64 Hz, 6H), 2.20 (s, 3H), 3.32-3.51 (m, 4H), 3.64-3.81 (m, 4H), 4.29 (s, 2H), 4.48-4.63 (m, 1H), 4.91 (s, 2H), 7.43-7.58 (m, 3H), 8.04 (dd, J=7.81, 1.56 Hz, 2H). MS [M+H]⁺ 477.7 (ESI). HRMS m/z calcd for C₂₄H₂₉N₈O₃ [M+H]⁺ 477.2357, found 477.2362.

### Example 146

### 4-(4-acetylpiperazin-1-yl)-2-[(1-phenylpyrazol-4-yl)methylamino]-8-propan-2-yl-3,5,8-triazabicyclo[4.3.0]nona-1,3,5-trien-7-one

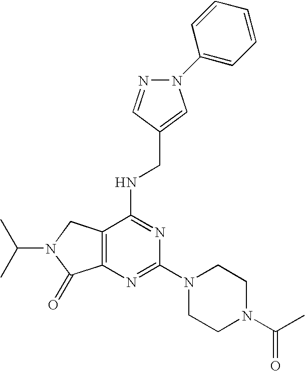

Following a procedure similar to that described in General Procedure 1, the title compound was obtained as a solid (35.0 mg, 21.6%) following purification by preparative LCMS (gradient 35-55% CH₃CN in H₂O containing 10 mM NH₄HCO₃) and lyophilization from CH₃CN/H₂O. HPLC purity: >95% (215 nm), >95% (254 nm), >96% (280 nm); Rₜ: 1.44 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=7.03 Hz, 6H), 2.12 (s, 3H), 3.53-3.58 (m, 2H), 3.59-3.64 (m, 2H), 3.86 (dd, J=6.25, 4.30 Hz, 2H), 3.91 (dd, J=6.25, 4.30 Hz, 2H), 4.20 (s, 2H), 4.47-4.56 (m, 1H), 4.64 (s, 2H), 7.30 (t, J=7.42 Hz, 1H), 7.43-7.49 (m, 2H), 7.66-7.73 (m, 3H), 8.19 (s, 1H). MS [M+H]⁺ 475.2 (ESI). HRMS m/z calcd for C₂₅H₃₁N₈O₂ [M+H]⁺ 475.2564, found 475.2567.

### Example 147

### 4-(4-acetylpiperazin-1-yl)-2-[(3-phenyl-1,2-oxazol-5-yl)methylamino]-8-propan-2-yl-3,5,8-triazabicyclo[4.3.0]nona-1,3,5-trien-7-one

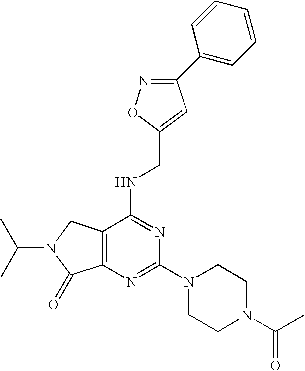

Following a procedure similar to that described in General Procedure 1, the title compound was obtained as a solid (42.0 mg, 25.9%) following purification by preparative LCMS (gradient 35-55% CH₃CN in H₂O containing 10 mM NH₄HCO₃) and lyophilization from CH₃CN/H₂O₀ HPLC purity: >99% (215 nm), >99% (254 nm), >99% (280 nm); Rₜ: 1.54 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=6.64 Hz, 6H), 2.08 (s, 3H), 3.45-3.53 (m, 2H), 3.53-3.58 (m, 2H), 3.76-3.82 (m, 2H), 3.83-3.91 (m, 2H), 4.25 (s, 2H), 4.45-4.61 (m, 1H), 4.84 (s, 2H), 6.74 (s, 1H), 7.37-7.53 (m, 3H), 7.79 (dd, J=6.84, 2.93 Hz, 2H). MS [M+H]⁺ 476.2 (ESI). HRMS m/z calcd for C₂₅H₃₀N₇O₃ [M+H]⁺ 476.2404, found 476.2402.

### Example 148

### 4-(4-acetylpiperazin-1-yl)-2-[(5-phenyl-1,3,4-oxadiazol-2-yl)methylamino]-8-propan-2-yl-3,5,8-triazabicyclo[4.3.0]nona-1,3,5-trien-7-one

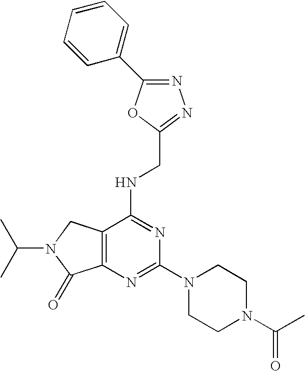

Following a procedure similar to that described in General Procedure 1, the title compound was obtained as a solid (35.0 mg, 21.6%) following purification by silica gel chromatography (gradient 2-20% MeOH in EtOAc), preparative LCMS (gradient 35-55% CH₃CN in H₂O containing 10 mM NH₄HCO₃) and lyophilization from CH₃CN/H₂O. HPLC purity: >99% (215 nm), >99% (254 nm), >99% (280 nm); Rₜ: 1.31 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.32 (d, J=6.64 Hz, 6H), 2.05 (s, 3H), 3.39-3.46 (m, 2H), 3.46-3.53 (m, 2H), 3.70-3.76 (2H), 3.77-3.82 (m, 2H), 4.28 (s, 2H), 4.45-4.60 (m, 1H), 4.93 (s, 2H), 7.48-7.65 (m, 3H), 8.01 (dd, J=8.20, 1.56 Hz, 2H). MS [M+H]⁺ 477.2 (ESI). HRMS m/z calcd for C₂₄H₂₉N₈O₃ [M+H]⁺ 477.2357, found 477.2363.

### Example 149

### 4-(4-acetylpiperazin-1-yl)-2-[1-[1-(2-fluorophenyl)pyrazol-4-yl]ethylamino]-8-propan-2-yl-3,5,8-triazabicyclo[4.3.0]nona-1,3,5-trien-7-one

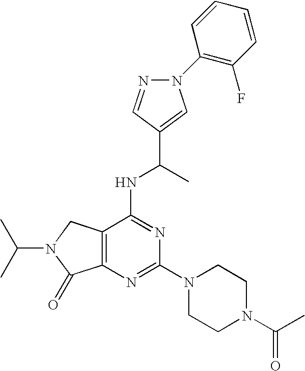

Following a procedure similar to that described in General Procedure 1, the title compound was obtained as a solid (87 mg, 50.3%) following purification by silica gel chromatography (gradient 2-20% MeOH in EtOAc), preparative LCMS (gradient 25-45% CH₃CN in H₂O containing 10 mM NH₄HCO₃) and lyophilization from CH₃CN/H₂O. HPLC purity: >99% (215 nm), >99% (254 nm), >99% (280 nm); Rₜ: 1.57 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (dd, J=6.84, 2.93 Hz, 6H), 1.63 (d, J=7.03 Hz, 3H), 2.22 (s, 3H), 3.55 (dd, J=6.45, 3.32 Hz, 2H), 3.58-3.62 (m, 2H), 3.80-3.85 (m, 2H), 3.86-3.91 (m, 2H), 4.21 (s, 2H), 4.47-4.57 (m, 1H), 5.47-5.56 (m, 1H), 7.27-7.42 (m, 3H), 7.69-7.74 (m, 1H), 7.75 (s, 1H), 8.04 (d, J=2.73 Hz, 1H). MS [M+H]⁺ 507.2 (ESI). HRMS m/z calcd for C₂₆H₃₂FN₈O₂ [M+H]⁺ 507.2626, found 507.2630.

### Example 150

### 4-(4-acetylpiperazin-1-yl)-2-[1-(1-phenylpyrazol-4-yl)ethylamino]-8-propan-2-yl-3,5,8-triazabicyclo[4.3.0]nona-1,3,5-trien-7-one

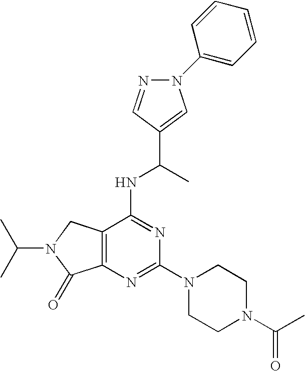

Following a procedure similar to that described in General Procedure 1, the title compound was obtained as a solid (39.0 mg, 9.09%) following purification by silica gel chromatography (gradient 2-20% MeOH in EtOAc), preparative LCMS (gradient 25-45% CH₃CN in H₂O containing 10 mM NH₄HCO₃) and lyophilization from CH₃CN/H₂O. HPLC purity: >99% (215 nm), >99% (254 nm), >99% (280 nm); Rₜ: 1.57 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (dd, J=6.64, 2.34 Hz, 6H), 1.64 (d, J=7.03 Hz, 3H), 2.11 (s, 3H), 3.49-3.65 (m, 4H), 3.76-3.94 (m, 4H), 4.22 (s, 2H), 4.43-4.60 (m, 1H), 5.41-5.60 (m, 1H), 7.27-7.33 (m, 1H), 7.43-7.49 (m, 2H), 7.67-7.72 (m, 3H), 8.19 (s, 1H). MS [M+H]⁺ 489.2 (ESI). HRMS m/z calcd for C₂₆H₃₃N₈O₂ [M+H]⁺ 489.2721. Found: 489.2719.

### Example 151

### 4-(4-acetylpiperazin-1-yl)-2-(6,7-diazabicyclo[3.3.0]octa-7,9-dien-8-ylmethylamino)-8-propan-2-yl-3,5,8-triazabicyclo[4.3.0]nona-1,3,5-trien-7-one

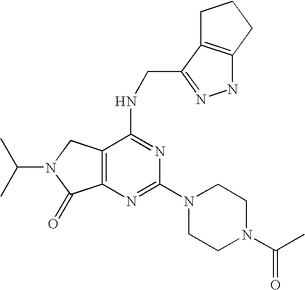

Following a procedure similar to that described in General Procedure 1, the title compound was obtained as a solid (50.0 mg, 25.5%) following purification by silica gel chromatography (gradient 3-30% MeOH in EtOAc), preparative LCMS (gradient 35-55% CH₃CN in H₂O containing 10 mM NH₄HCO₃) and lyophilization from CH₃CN/H₂O. HPLC purity: >95% (215 nm), >95% (254 nm), >95% (280 nm); Rₜ: 1.00 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.64 Hz, 6H), 2.12 (s, 3H), 2.31-2.50 (m, 4H), 2.56-2.68 (m, 2H), 3.45-3.61 (m, 4H), 3.78 (dd, J=6.25, 3.91 Hz, 2H), 3.82-3.87 (m, 2H), 4.19 (s, 2H), 4.47-4.56 (m, 1H), 4.63 (s, 2H). MS [M+H]⁺ 439.3 (ESI). HRMS m/z calcd for C₂₂H₃₁N₈O₂ [M+H]⁺ 439.2554. Found: 439.2562.

### Example 152

### 4-(4-acetylpiperazin-1-yl)-2-[(1-cyclopentyl-3-methyl-pyrazol-4-yl)methylamino]-8-propan-2-yl-3,5,8-triazabicyclo[4.3.0]nona-2,4,10-trien-7-one

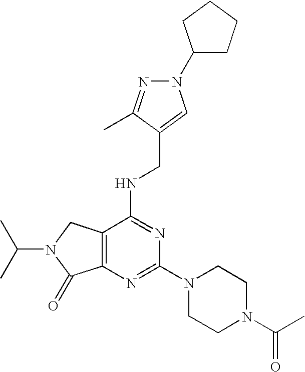

Following a procedure similar to that described in General Procedure 1, the title compound was obtained as a solid (79 mg, 36.8%) following purification by silica gel chromatography (gradient 3-30% MeOH in EtOAc), preparative LCMS (gradient 25-45% CH₃CN in H₂O containing 10 mM NH₄HCO₃) and lyophilization from CH₃CN/H₂O. HPLC purity: >98% (215 nm), >97% (254 nm), >97% (280 nm); Rₜ: 1.40 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (d, J=6.64 Hz, 6H), 1.64-1.74 (m, 2H), 1.77-1.95 (m, 4H), 2.04-2.16 (m, 5H), 2.22 (s, 3H), 3.58 (dd, J=6.25, 4.30 Hz, 2H), 3.60-3.65 (m, 2H), 3.82-3.87 (m, 2H), 3.88-3.93 (m, 2H), 4.15 (s, 2H), 4.46-4.59 (m, 4H), 7.55 (s, 1H). MS [M+H]⁺ 481.2 (ESI). HRMS m/z calcd for C₂₅H₃₇N₈O₂ [M+H]⁺ 481.3034. Found: 481.3032.

### Example 153

### 4-(4-acetylpiperazin-1-yl)-2-[(1-methyl-5-phenyl-pyrazol-3-yl)methylamino]-8-propan-2-yl-3,5,8-triazabicyclo[4.3.0]nona-1,3,5-trien-7-one

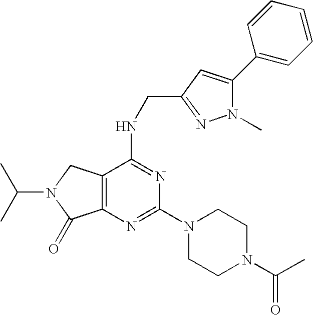

Following a procedure similar to that described in General Procedure 1, the title compound was obtained as a solid (43.0 mg, 19.69%) following purification by silica gel chromatography (gradient 2-20% MeOH in EtOAc), preparative LCMS (gradient 35-55% CH₃CN in H₂O containing 10 mM NH₄HCO₃) and lyophilization from CH₃CN/H₂O. HPLC purity: >99% (215 nm), >99% (254 nm), >99% (280 nm); Rₜ: 1.50 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.30 (d, J=6.64 Hz, 6H), 2.11 (s, 3H), 3.49-3.61 (m, 4H), 3.79-3.85 (m, 5H), 3.85-3.91 (m, 2H), 4.22 (s, 2H), 4.46-4.58 (m, 1H), 4.67 (s, 2H), 6.31 (s, 1H), 7.36-7.51 (m, 5H). MS [M+H]⁺ 489.2 (ESI). HRMS m/z calcd for C₂₆H₃₃N₈O₂ [M+H]⁺ 489.2721. Found: 489.2721.

### Example 154

### 4-(4-acetylpiperazin-1-yl)-2-[(2-methyl-5-phenyl-pyrazol-3-yl)methylamino]-8-propan-2-yl-3,5,8-triazabicyclo[4.3.0]nona-1,3,5-trien-7-one

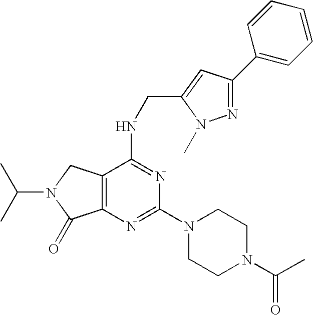

Following a procedure similar to that described in General Procedure 1, the title compound was obtained as a solid (43.0 mg, 19.69%) following purification by silica gel chromatography (gradient 2-20% MeOH in EtOAc), preparative LCMS (gradient 35-55% CH₃CN in H₂O containing 10 mM NH₄HCO₃) and lyophilization from CH₃CN/H₂O. HPLC purity: >99% (215 nm), >99% (254 nm), >99% (280 nm); Rₜ: 1.49 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=7.03 Hz, 6H), 2.09 (s, 3H), 3.47-3.54 (m, 2H), 3.55-3.62 (m, 2H), 3.77-3.93 (m, 7H), 4.22 (s, 2H), 4.42-4.60 (m, 1H), 4.79 (s, 2H), 6.61 (s, 1H), 7.20-7.31 (m, 1H), 7.36 (t, J=7.42 Hz, 2H), 7.71 (d, J=7.03 Hz, 2H). MS [M+H]⁺ 489.2 (ESI). HRMS m/z calcd for C₂₆H₃₃N₈O₂ [M+H]⁺ 489.2721. Found: 489.2722.

### Example 155

### 4-(4-acetylpiperazin-1-yl)-2-(1,7-diazabicyclo[4.3.0]nona-2,4,6,8-tetraen-8-ylmethylamino)-8-propan-2-yl-3,5,8-triazabicyclo[4.3.0]nona-1,3,5-trien-7-one

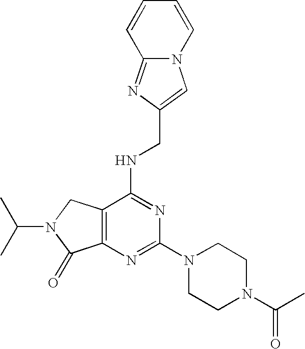

Following a procedure similar to that described in General Procedure 1, the title compound was obtained as a solid (30.0 mg, 12.66%) following purification by silica gel chromatography (gradient 2-20% MeOH in EtOAc), preparative LCMS (gradient 25-45% CH₃CN in H₂O containing 10 mM NH₄HCO₃) and lyophilization from CH₃CN/H₂O. HPLC purity: >98% (215 nm), >98% (254 nm), >98% (280 nm); Rₜ: 0.78 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (d, J=6.64 Hz, 6H), 2.08 (s, 3H), 3.46 (d, J=10.94 Hz, 2H), 3.50-3.54 (m, 2H), 3.76 (dd, J=6.45, 3.71 Hz, 2H), 3.82 (d, J=10.55 Hz, 2H), 4.22 (s, 2H), 4.45-4.55 (m, 1H), 4.79 (s, 2H), 6.85 (t, J=6.84 Hz, 1H), 7.26 (t, J=8.40 Hz, 1H), 7.46 (d, J=8.59 Hz, 1H), 7.72 (s, 1H), 8.33 (d, J=6.25 Hz, 1H). MS [M+H]⁺ 449.2 (ESI). HRMS m/z calcd for C₂₃H₂₉N₈O₂ [M+H]⁺ 449.2408. Found: 449.2405.

### Example 156

### (R)-2-(4-acetylpiperazin-1-yl)-4-(1-(3-ethoxyphenyl)ethylamino)-6-isopropyl-5H-pyrrolo[3,4-d]pyrimidin-7(6H)-one

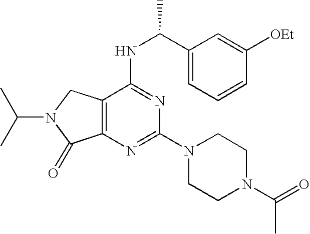

Following a procedure similar to that described in General Procedure 1 and after purification by silica gel chromatography (100% EtOAc to 40% MeOH in EtOAc) followed by preparative LCMS (high PH), the title compound (155 mg, 82%) was obtained as a solid. Purity: >99% (215 nm), >99% (254 nm), >99% (280 nm); Rₜ: 1.70 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (d, J=6.64 Hz, 6H), 1.33 (d, J=7.03 Hz, 3H), 1.51 (d, J=7.03 Hz, 3H), 2.07 (s, 3H), 3.32-3.41 (m, 3H), 3.42-3.54 (m, 1H), 3.61-3.74 (m, 3H), 3.72-3.85 (m, 1H), 3.97 (q, J=6.90 Hz, 2H), 4.23 (s, 2H), 4.41-4.56 (m, 1H), 5.15 (q, J=6.77 Hz, 1H), 6.71 (dd, J=8.01, 2.15 Hz, 1H), 6.83-6.92 (m, 2H), 7.16 (t, J=7.81 Hz, 1H). M.S. (found): 467.3 (ESI) (MH⁺).

### Example 157

### 2-(4-acetylpiperazin-1-yl)-4-((1-(4-ethoxyphenyl)ethyl)(methyl)amino)-6-isopropyl-5H-pyrrolo[3,4-d]pyrimidin-7(6H)-one

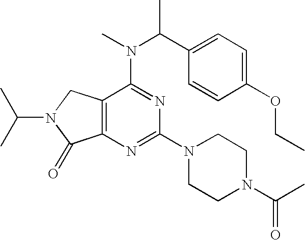

Following a procedure similar to that described in General Procedure 1 and after purification by silica gel chromatography (100% EtOAc to 40% MeOH in EtOAc) followed by preparative LCMS (high pH), the title compound (110 mg, 40%) was obtained as a solid. Purity: >99% (215 nm), >99% (254 nm), >99% (280 nm); Rₜ: 1.90 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.27 (dd, J=6.84, 3.71 Hz, 6H), 1.34 (t, J=7.03 Hz, 3H), 1.56 (d, J=7.03 Hz, 3H), 2.11 (s, 3H), 2.88 (s, 3H), 3.49-3.63 (m, 4H), 3.74-3.82 (m, 2H), 3.82-3.90 (m, 2H), 3.98 (q, J=6.77 Hz, 2H), 4.41-4.54 (m, 1H), 4.55-4.60 (m, 2H), 6.05 (s, 1H), 6.86 (d, J=8.59 Hz, 2H), 7.21 (d, J=8.59 Hz, 2H). M.S. (found): 481.2 (ESI) (MH⁺).

### Example 158

### (R)-2-(4-acetylpiperazin-1-yl)-4-(1-(4-ethoxy-3-fluorophenyl)ethylamino)-6-isopropyl-5H-pyrrolo[3,4-d]pyrimidin-7(6H)-one

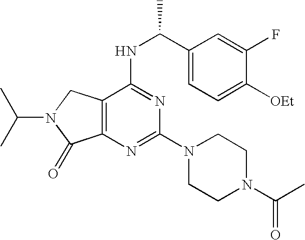

Following a procedure similar to that described in General Procedure 1, starting from (R)-1-(4-ethoxy-3-fluorophenyl)ethanamine hydrochloride (Intermediate 40) and after purification by silica gel chromatography (100% EtOAc to 40% MeOH in EtOAc) followed by preparative LCMS (high pH), the title compound (155 mg, 79%) was obtained as a solid. HPLC purity: >99% (215 nm), >99% (254 nm), >99% (280 nm); Rₜ: 1.72 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (d, J=6.64 Hz, 6H), 1.34 (t, J=7.03 Hz, 3H), 1.50 (d, J=7.03 Hz, 3H), 2.08 (s, 3H), 3.34-3.58 (m, 4H), 3.60-3.85 (m, 4H), 4.03 (q, J=7.03 Hz, 2H), 4.22 (s, 2H), 4.42-4.54 (m, 1H), 5.17 (q, J=6.90 Hz, 1H), 6.97 (t, J=8.79 Hz, 1H), 7.02-7.12 (m, 2H). M.S. (found): 485.2 (ESI) (MH⁺).

### Example 159

### (R)-2-(4-acetylpiperazin-1-yl)-4-(1-(3-chloro-4-ethoxyphenyl)ethylamino)-6-isopropyl-5H-pyrrolo[3,4-d]pyrimidin-7(6H)-one

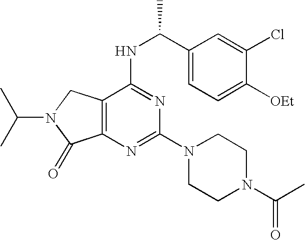

Following a procedure similar to that described in General Procedure 1, starting from (R)-1-(3-chloro-4-ethoxyphenyl)ethanamine hydrochloride (Intermediate 41) and after purification by silica gel chromatography (100% EtOAc to 40% MeOH in EtOAc) followed by preparative LCMS (high pH), the title compound (115 mg, 67%) was obtained as a solid. HPLC purity: >99% (215 nm), >99% (254 nm), >99% (280 nm); Rₜ: 1.86 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.28 (dd, J=6.84, 1.76 Hz, 6H), 1.36 (t, J=7.03 Hz, 3H), 1.50 (d, J=7.03 Hz, 3H), 2.08 (s, 3H), 3.31-3.57 (m, 4H), 3.60-3.86 (m, 4H), 4.03 (q, J=7.03 Hz, 2H), 4.22 (d, J=1.95 Hz, 2H), 4.41-4.54 (m, 1H), 5.13 (q, J=7.03 Hz, 1H), 6.94 (d, J=8.59 Hz, 1H), 7.21 (dd, J=8.59, 1.95 Hz, 1H), 7.35 (d, J=2.34 Hz, 1H). M.S. (found): 501.2 (ESI) (MH⁺).

### Example 160

### (R)-2-(4-acetylpiperazin-1-yl)-4-(1-(2,3-dihydrobenzofuran-5-yl)ethylamino)-6-isopropyl-5H-pyrrolo[3,4-d]pyrimidin-7(6H)-one

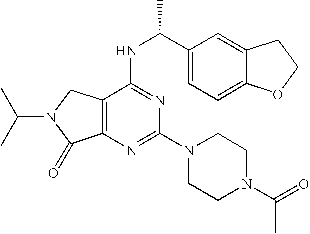

Following a procedure similar to that described in General Procedure 1 and after purification by preparative LCMS (high pH), the title compound (80 mg, 42%) was obtained as a solid. HPLC purity: >95% (215 nm), >96% (254 nm), >97% (280 nm); Rₜ: 1.53 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.22 (d, J=6.64 Hz, 6H), 1.63 (d, J=6.64 Hz, 3H), 2.11 (s, 3H), 3.16 (t, J=8.79 Hz, 2H), 3.49 (s, 2H), 3.57-3.69 (m, 3H), 3.79 (s, 3H), 3.89 (s, 2H), 4.33 (s, 1H), 4.47-4.56 (m, 3H), 5.16 (s, 1H), 6.69 (d, J=7.81 Hz, 1H), 7.08 (d, J=7.81 Hz, 1H), 7.26 (s, 1H). M.S. (found): 465.2 (ESI) (MH⁺). Accurate [M+H] OBS=465.26131.

### Example 161

### (R)-2-(4-(1-(2-(4-acetylpiperazin-1-yl)-6-isopropyl-7-oxo-6,7-dihydro-5H-pyrrolo[3,4-d]pyrimidin-4-ylamino)ethyl)phenyl)-2-methylpropanenitrile

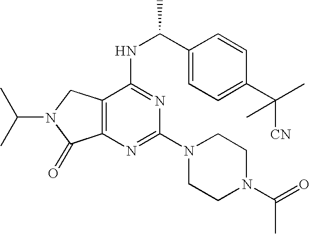

Following a procedure similar to that described in General Procedure 1 and after purification by preparative LCMS (high pH), the title compound (137 mg, 69%) was obtained as a solid. HPLC purity: >99% (215 nm), >99% (254 nm), >99% (280 nm); Rₜ: 1.67 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.22 (d, J=6.64 Hz, 6H), 1.60-1.74 (m, 9H), 2.10 (s, 3H), 3.34-3.94 (m, 10H), 4.37 (s, 1H), 4.46-4.57 (m, 1H), 5.20 (s, 1H), 7.35-7.49 (m, 4H). M.S. (found): 490.2 (ESI) (MH⁺). Accurate [M+H] OBS=490.29162.

### Example 162

### 2-(4-acetylpiperazin-1-yl)-4-((cyclopropylmethyl)(4-ethoxybenzyl)amino)-6-isopropyl-5H-pyrrolo[3,4-d]pyrimidin-7(6H)-one

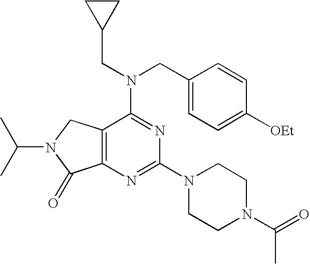

To a solution of 2-chloro-4-((cyclopropylmethyl)(4-ethoxybenzyl)amino)-6-isopropyl-5H-pyrrolo[3,4-d]pyrimidin-7(6H)-one (Intermediate 116) (140 mg, 0.34 mmol) in n-butanol (2 mL) was added 1-(piperazin-1-yl)ethanone (43.2 mg, 0.34 mmol) followed by DIPEA (0.059 mL, 0.34 mmol) at rt. The reaction mixture was heated in a microwave reactor at 160° C. for 60 minutes. After cooling to rt, the mixture was concentrated under reduced pressure, and the residue was purified by preparative LCMS (high pH) to afford the title compound (112 mg, 66%). HPLC purity: >98% (215 nm), >99% (254 nm), >99% (280 nm); Rₜ: 2.11 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 0.22 (t, J=5.27 Hz, 2H), 0.54 (d, J=7.42 Hz, 2H), 1.04 (d, J=6.64 Hz, 1H), 1.19 (d, J=6.64 Hz, 6H), 1.39 (t, J=7.03 Hz, 3H), 2.07-2.17 (m, 3H), 3.37-3.46 (m, 2H), 3.53 (s, 2H), 3.65 (d, J=5.08 Hz, 2H), 3.82 (s, 2H), 3.91-4.05 (m, 4H), 4.28 (s, 2H), 4.60 (s, 1H), 4.81 (s, 2H), 6.84 (d, J=8.59 Hz, 2H), 7.08 (d, J=8.59 Hz, 2H). M.S. (found): 507.2 (ESI) (MH⁺). Accurate [M+H] OBS=507.30720.

### Example 163

### 2-(4-(1-(2-(4-acetylpiperazin-1-yl)-6-isopropyl-7-oxo-6,7-dihydro-5H-pyrrolo[3,4-d]pyrimidin-4-ylamino)ethyl)-2-fluorophenyl)-2-methylpropanenitrile

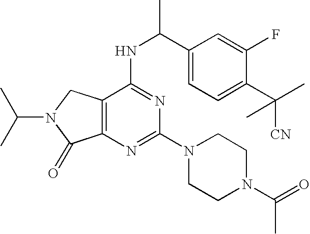

Following a procedure similar to that described in General Procedure 1 and after purification by preparative LCMS (high pH), the title compound (363 mg, 71%) was obtained as a solid. HPLC purity: >98% (215 nm), >98% (254 nm), >98% (280 nm); Rₜ: 1.73 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.22 (d, J=6.64 Hz, 6H), 1.61 (d, J=7.03 Hz, 3H), 1.73-1.77 (m, 6H), 2.09 (s, 3H), 3.36-3.62 (m, 5H), 3.63-3.88 (m, 4H), 4.29 (d, J=12.89 Hz, 2H), 4.54 (s, 1H), 5.15-5.26 (m, 1H), 7.09-7.20 (m, 2H), 7.36-7.43 (m, 1H). M.S. (found): 508.3 (ESI) (MH⁺). Accurate [M+H] OBS=508.28342.

### Example 164

### 2-(4-acetylpiperazin-1-yl)-6-isopropyl-4-(2-methyl-1-p-tolylpropan-2-ylamino)-5H-pyrrolo[3,4-d]pyrimidin-7(6H)-one

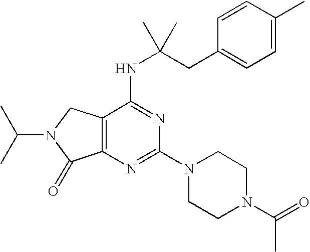

Following a procedure similar to that described in General Procedure 2 and after purification by preparative LCMS (high pH), the title compound (18 mg, 90%) was obtained as a solid. HPLC purity: >99% (215 nm), >99% (254 nm), >99% (280 nm); Rₜ: 1.97 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.23 (d, J=6.64 Hz, 6H), 1.51 (s, 6H), 2.06-2.19 (s, 3H), 2.29 (s, 3H), 3.19 (s, 2H), 3.43-4.15 (m, 1H), 4.47 (s, 1H), 6.94 (d, J=7.81 Hz, 2H), 7.04 (d, J=7.81 Hz, 2H). M.S. (found): 465.2 (ESI) (MH⁺). Accurate [M+H] OBS=465.29738.

### Example 165

### (R)-2-(4-acetylpiperazin-1-yl)-4-(1-(4-iodophenyl)ethylamino)-6-isopropyl-5H-pyrrolo[3,4-d]pyrimidin-7(6H)-one

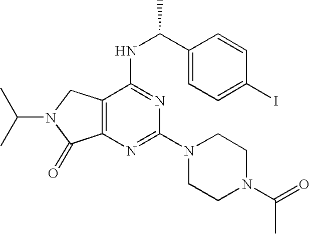

Following a procedure similar to that described in General Procedure 1 and after purification by preparative LCMS (high pH), the title compound (117 mg, 52%) was obtained as a solid. HPLC purity: >99% (215 nm), >99% (254 nm), >99% (280 nm); Rₜ: 1.83 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.14-1.27 (m, 6H), 1.58 (d, J=6.64 Hz, 3H), 2.09 (s, 3H), 3.29-3.45 (m, 2H), 3.54 (s, 2H), 3.65 (s, 2H), 3.77 (s, 3H), 4.22 (d, J=8.98 Hz, 2H), 4.51-4.61 (m, 1H), 5.14 (s, 1H), 7.10 (d, J=8.20 Hz, 2H), 7.61 (d, J=8.20 Hz, 2H). M.S. (found): 549.0 (ESI) (MH⁺). Accurate [M+H] OBS=549.14673.

### Example 166

### (R)-2-(4-acetylpiperazin-1-yl)-6-isopropyl-4-(1-(5,6,7,8-tetrahydronaphthalen-2-yl)ethylamino)-5H-pyrrolo[3,4-d]pyrimidin-7(6H)-one

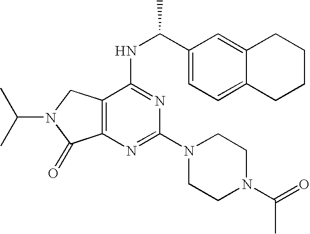

Following a procedure similar to that described in General Procedure 1 and after purification by preparative LCMS (high pH), the title compound (110 mg, 57%) was obtained as a solid. HPLC purity: >99% (215 nm), >99% (254 nm), >99% (280 nm); Rₜ: 2.00 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.21 (d, J=6.64 Hz, 6H), 1.59 (d, J=6.64 Hz, 3H), 1.75 (s, 4H), 2.10 (s, 3H), 2.71 (s, 4H), 3.38-3.48 (m, 2H), 3.51-3.66 (m, 2H), 3.71-3.93 (m, 5H), 4.16 (s, 2H), 4.55 (d, J=6.64 Hz, 1H), 5.15 (s, 1H), 6.95-7.11 (m, 3H). M.S. (found): 477.2 (ESI) (MH⁺). Accurate [M+H] OBS=477.29734.

### Example 167

### (R)-2-(4-acetylpiperazin-1-yl)-4-(1-(4-ethoxy-3-methylphenyl)ethylamino)-6-isopropyl-5H-pyrrolo[3,4-d]pyrimidin-7(6H)-one

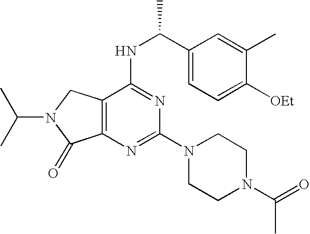

Following a procedure similar to that described in General Procedure 1 and after purification by preparative LCMS (high pH), the title compound (105 mg, 54%) was obtained as a solid. HPLC purity: >99% (215 nm), >99% (254 nm), >99% (280 nm); Rₜ: 1.89 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.21 (d, J=6.64 Hz, 6H), 1.38 (t, J=6.84 Hz, 3H), 1.62 (d, J=6.64 Hz, 3H), 2.10 (s, 3H), 2.14-2.20 (m, 3H), 3.47 (s, 2H), 3.63 (s, 2H), 3.79 (s, 2H), 3.89 (s, 2H), 3.97 (q, J=6.77 Hz, 3H), 4.28 (s, 2H), 4.52 (s, 1H), 5.13 (s, 1H), 6.73 (d, J=8.20 Hz, 1H), 7.09-7.17 (m, 2H). M.S. (found): 481.2 (ESI) (MH⁺). Accurate [M+H] OBS=481.29193.

### Example 168

### (R)-2-(4-acetylpiperazin-1-yl)-4-(1-(4-ethoxy-2-methylphenyl)ethylamino)-6-isopropyl-5H-pyrrolo[3,4-d]pyrimidin-7(6H)-one

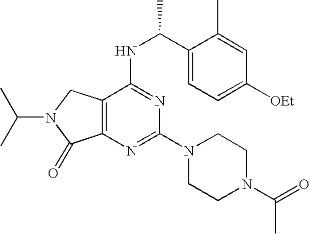

Following a procedure similar to that described in General Procedure 1, starting from (R)-1-(4-ethoxy-2-methylphenyl)ethanamine hydrochloride (Intermediate 43) and after purification by preparative LCMS (high pH), the title compound (131 mg, 67%) was obtained as a solid. HPLC purity: >99% (215 nm), >99% (254 nm), >99% (280 nm); Rₜ: 1.80 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.20 (d, J=6.64 Hz, 6H), 1.36 (t, J=7.03 Hz, 3H), 1.53 (d, J=6.25 Hz, 3H), 2.09 (s, 3H), 2.32 (s, 3H), 3.39 (d, J=3.91 Hz, 2H), 3.51-3.61 (m, 2H), 3.65-3.78 (m, 3H), 3.82 (s, 2H), 3.96 (q, J=6.77 Hz, 2H), 4.11 (s, 2H), 4.50-4.62 (m, 1H), 5.36 (s, 1H), 6.64-6.72 (m, 2H), 7.25 (s, 1H). M.S. (found): 481.2 (ESI) (MH⁺). Accurate [M+H] OBS=481.29214.

### Example 169

### (R)-2-(4-acetylpiperazin-1-yl)-4-(1-(2,2-dimethylchroman-6-yl)ethylamino)-6-isopropyl-5H-pyrrolo[3,4-d]pyrimidin-7(6H)-one

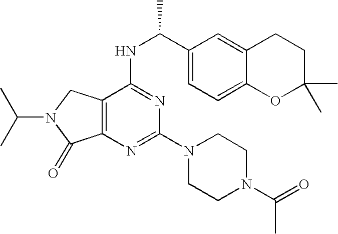

Following a procedure similar to that described in General Procedure 1, starting from (R)-1-(2,2-dimethylchoman-6-yl)ethanamine hydrochloride (Intermediate 44) and after purification by preparative LCMS (high pH), the title compound (44 mg, 71%) was obtained as a solid. HPLC purity: >99% (215 nm), >99% (254 nm), >99% (280 nm); Rₜ: 1.92 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.20 (d, J=6.64 Hz, 6H), 1.29 (s, 6H), 1.57 (d, J=6.64 Hz, 3H), 1.76 (t, J=6.84 Hz, 2H), 2.10 (s, 3H), 2.72 (t, J=6.64 Hz, 2H), 3.44 (s, 2H), 3.61 (s, 3H), 3.79 (s, 2H), 3.86 (s, 2H), 4.15 (s, 2H), 4.50-4.61 (m, 1H), 5.15 (s, 1H), 6.70 (d, J=7.81 Hz, 1H), 7.04 (s, 2H). M.S. (found): 507.2 (ESI) (MH⁺). Accurate [M+H] OBS=507.30775.

### Example 170

### (R)-2-(4-acetylpiperazin-1-yl)-4-(1-(3,4-dimethylphenyl)ethylamino)-6-isopropyl-5H-pyrrolo[3,4-d]pyrimidin-7(6H)-one

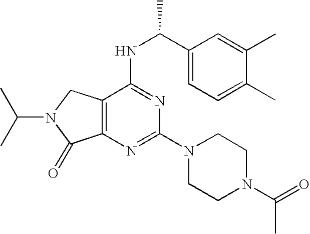

Following a procedure similar to that described in General Procedure 1, starting from (R)-1-(3,4-dimethylphenyl)ethanamine hydrochloride (Intermediate 45) and after purification by preparative LCMS (high pH), the title compound (90 mg, 49%) was obtained as a solid. HPLC purity: >95% (215 nm), >99% (254 nm), >99% (280 nm); Rₜ: 1.75 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.19 (d, J=7.03 Hz, 6H), 1.56 (d, J=6.64 Hz, 3H), 2.09 (s, 3H), 2.21 (d, J=5.47 Hz, 6H), 3.39 (s, 2H), 3.49-3.63 (m, 2H), 3.75 (s, 2H), 3.82 (d, J=5.47 Hz, 2H), 4.12 (s, 2H), 4.51-4.64 (m, 1H), 5.16 (s, 1H), 5.42-5.60 (m, 1H), 7.06 (s, 2H), 7.09 (s, 1H). M.S. (found): 451.2 (ESI) (MH⁺). Accurate [M+H] OBS=451.28163.

### Example 171

### (R)-2-(4-acetylpiperazin-1-yl)-4-(1-(4-chloro-3-(trifluoromethyl)phenyl)ethylamino)-6-isopropyl-5H-pyrrolo[3,4-d]pyrimidin-7(6H)-one

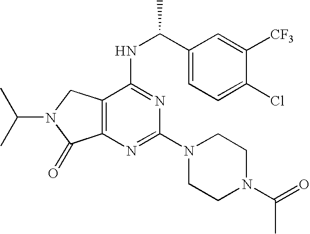

Following a procedure similar to that described in General Procedure 1, starting from (R)-1-(4-chloro-3-(trifluoromethyl)phenyl)ethanamine hydrochloride (Intermediate 46) and after purification by preparative LCMS (high pH), the title compound (80 mg, 38%) was obtained as a solid. HPLC purity: >99% (215 nm), >99% (254 nm), >99% (280 nm); Rₜ: 1.95 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.20 (d, J=6.64 Hz, 6H), 1.59 (d, J=7.03 Hz, 3H), 2.07 (s, 3H), 3.28 (d, J=5.08 Hz, 2H), 3.32-3.42 (m, 2H), 3.45-3.61 (m, 2H), 3.65-3.77 (m, 2H), 4.23 (d, J=9.77 Hz, 2H), 4.50-4.63 (m, 1H), 5.15-5.23 (m, 1H), 6.26 (s, 1H), 7.38-7.43 (m, 1H), 7.50 (d, J=8.20 Hz, 1H), 7.65 (s, 1H). M.S. (found): 525.3 (ESI) (MH⁺). Accurate [M+H] OBS=525.19888.

### Example 172

### (R)-2-(4-acetylpiperazin-1-yl)-4-(1-(2,3-dihydro-1H-inden-5-yl)ethylamino)-6-isopropyl-5H-pyrrolo[3,4-d]pyrimidin-7(6H)-one

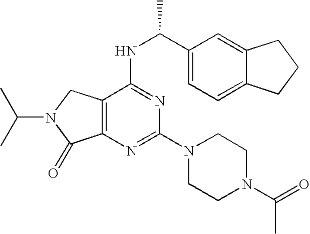

Following a procedure similar to that described in General Procedure 1 and after purification by preparative LCMS (high pH), the title compound (134 mg, 71%) was obtained as a solid. HPLC purity: >98% (215 nm), >99% (254 nm), >99% (280 nm); Rₜ: 1.80 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) [symbol] ppm 1.19 (d, J=6.64 Hz, 6H), 1.59 (d, J=7.03 Hz, 3H), 1.97-2.06 (m, 2H), 2.09 (s, 3H), 2.84 (t, J=7.42 Hz, 4H), 3.40 (s, 2H), 3.47-3.64 (m, 2H), 3.69-3.93 (m, 4H), 4.17 (s, 2H), 4.49-4.65 (m, 1H), 5.19 (s, 1H), 5.96 (s, 1H), 7.12 (q, J=7.81 Hz, 2H), 7.20 (s, 1H). M.S. (found): 463.3 (ESI) (MH⁺). Accurate [M+H] OBS=463.28182.

### Example 173

### (R)-2-(4-acetylpiperazin-1-yl)-4-(1-(2-ethoxyphenyl)ethylamino)-6-isopropyl-5H-pyrrolo[3,4-d]pyrimidin-7(6H)-one

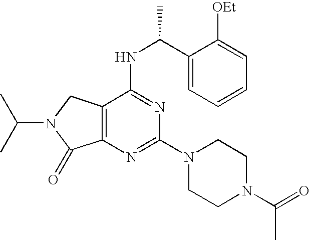

Following a procedure similar to that described in General Procedure 1 and after purification by preparative LCMS (high pH), the title compound (106 mg, 56%) was obtained as a solid. HPLC purity: >99% (215 nm), >99% (254 nm), >99% (280 nm); Rₜ: 1.71 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) [symbol] ppm 1.19 (d, J=6.64 Hz, 6H), 1.43 (t, J=6.84 Hz, 3H), 1.53 (d, J=6.64 Hz, 3H), 2.07 (s, 3H), 3.38 (s, 2H), 3.44-3.63 (m, 2H), 3.67-3.92 (m, 4H), 3.98-4.23 (m, 4H), 4.51-4.62 (m, 1H), 5.47 (s, 1H), 6.24 (s, 1H), 6.85 (t, J=8.01 Hz, 2H), 7.16 (t, J=7.42 Hz, 1H), 7.26 (s, 1H). M.S. (found): 467.3 (ESI) (MH⁺). Accurate [M+H] OBS=467.27586.

### Example 174

### (R)-2-(4-acetylpiperazin-1-yl)-4-(1-(3-ethoxy-4-methylphenyl)ethylamino)-6-isopropyl-5H-pyrrolo[3,4-d]pyrimidin-7(6H)-one

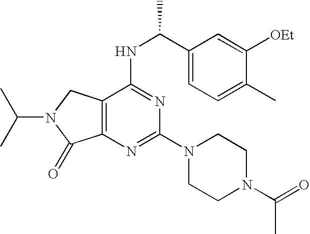

Following a procedure similar to that described in General Procedure 1, starting from (R)-1-(3-ethoxy-4-methylphenyl)ethanamine hydrochloride (Intermediate 47) and after purification by preparative LCMS (high pH), the title compound (110 mg, 56%) was obtained as a solid. HPLC purity: >99% (215 nm), >99% (254 nm), >99% (280 nm); Rₜ: 1.84 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) [symbol] ppm 1.20 (d, J=6.64 Hz, 6H), 1.38 (d, J=7.03 Hz, 3H), 1.60 (d, J=6.64 Hz, 3H), 2.09 (s, 3H), 2.16 (s, 3H), 3.42 (s, 2H), 3.58 (s, 2H), 3.70-3.91 (m, 4H), 3.95-4.05 (m, 2H), 4.18 (s, 2H), 4.49-4.60 (m, 1H), 5.16 (s, 1H), 6.78-6.86 (m, 2H), 7.05 (d, J=7.81 Hz, 1H). M.S. (found): 481.2 (ESI) (MH⁺).

### Example 175

### (R)-2-(4-acetylpiperazin-1-yl)-4-(1-(4-ethoxyphenyl)propylamino)-6-isopropyl-5H-pyrrolo[3,4-d]pyrimidin-7(6H)-one

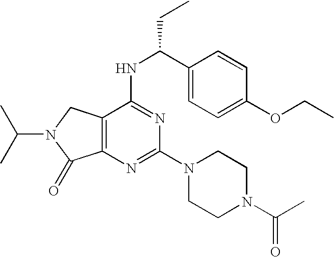

Following a procedure similar to that described in General Procedure 1 and after purification by preparative LCMS (gradient 35-55% CH₃CN in H₂O containing 10 mM NH₄HCO₃) and lyophilization from CH₃CN/H₂O, the title compound was obtained as a solid (120 mg, 49%). HPLC purity: >99% (215 nm), >99% (254 nm), >99% (280 nm); Rₜ: 1.77 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) [symbol] ppm 0.93 (t, J=7.42 Hz, 3H), 1.21-1.40 (m, 9H), 1.69-2.00 (m, 2H), 2.09 (s, 3H), 3.35-3.64 (m, 4H), 3.64-3.87 (m, 4H), 3.96 (q, J=7.03 Hz, 2H), 4.21 (s, 2H), 4.49 (quin, J=6.74 Hz, 1H), 4.93 (t, J=7.42 Hz, 1H), 6.81 (d, J=8.59 Hz, 2H), 7.23 (d, J=8.59 Hz, 2H). M.S. 482.1 (ESI) (MH⁺). HRMS m/z calcd for C₂₆H₃₆N₆O₃ [M+H]⁺ 481.2921, found 481.2117.

### Example 176

### (R)-2-(4-acetylpiperazin-1-yl)-4-(1-(4-isopropoxyphenyl)ethylamino)-6-isopropyl-5H-pyrrolo[3,4-d]pyrimidin-7(6H)-one

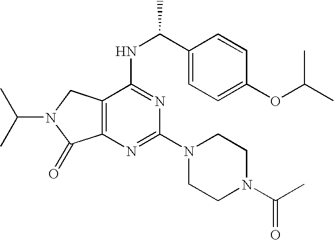

Following a procedure similar to that described in General Procedure 1 and after purification by preparative LCMS (gradient 35-55% CH₃CN in H₂O containing 10 mM NH₄HCO₃) and lyophilization from CH₃CN/H₂O, the title compound was obtained as a solid (135 mg, 55%). HPLC purity: >99% (215 nm), >99% (254 nm), >99% (280 nm); Rₜ: 1.76 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.25 (dd, J=13.28, 6.25 Hz, 12H), 1.50 (d, J=7.03 Hz, 3H), 2.08 (s, 3H), 3.34-3.59 (m, 4H), 3.59-3.90 (m, 4H), 4.20 (s, 2H), 4.38-4.60 (m, 2H), 5.18 (q, J=6.64 Hz, 1H), 6.80 (d, J=8.59 Hz, 2H), 7.24 (d, J=8.59 Hz, 2H). M.S. 482.1 (ESI) (MH⁺). HRMS m/z calcd for C₂₆H₃₆N₆O₃ [M+H]⁺ 481.2921, found 481.2122.

### Example 177

### (S)-2-(4-acetylpiperazin-1-yl)-4-(1-(4-ethoxyphenyl)-2,2,2-trifluoroethylamino)-6-isopropyl-5H-pyrrolo[3,4-d]pyrimidin-7(6H)-one

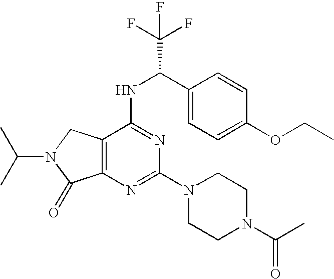

Following a procedure similar to that described in General Procedure 1 and after purification by preparative LCMS (gradient 35-55% CH₃CN in H₂O containing 10 mM NH₄HCO₃) and lyophilization from CH₃CN/H₂O, the title compound was obtained as a solid (50 mg, 41%). HPLC purity: >99% (215 nm), >99% (254 nm), >99% (280 nm); Rₜ: 1.76 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (t, J=7.03 Hz, 6H), 1.34 (t, J=6.84 Hz, 3H), 2.11 (s, 3H), 3.43-3.69 (m, 4H), 3.71-3.94 (m, 4H), 4.00 (q, J=7.03 Hz, 2H), 4.27 (s, 2H), 4.50 (quin, J=6.74 Hz, 1H), 6.10 (q, J=8.59 Hz, 1H), 6.79-6.99 (m, 2H), 7.44 (d, J=8.59 Hz, 2H). M.S. 521.3 (ESI) (MH⁺). HRMS m/z calcd for C₂₅H₃₁F₃N₆O₃ [M+H]⁺ 521.2482, found 521.2480.

### Example 178

### 2-(4-acetylpiperazin-1-yl)-4-(1-(2,3-dihydrobenzo[b][1,4]dioxin-2-yl)ethylamino)-6-isopropyl-5H-pyrrolo[3,4-d]pyrimidin-7(6H)-one (Isomer 1) and

### Example 179

### 2-(4-acetylpiperazin-1-yl)-4-(1-(2,3-dihydrobenzo[b][1,4]dioxin-2-yl)ethylamino)-6-isopropyl-5H-pyrrolo[3,4-d]pyrimidin-7(6H)-one (Isomer 2)

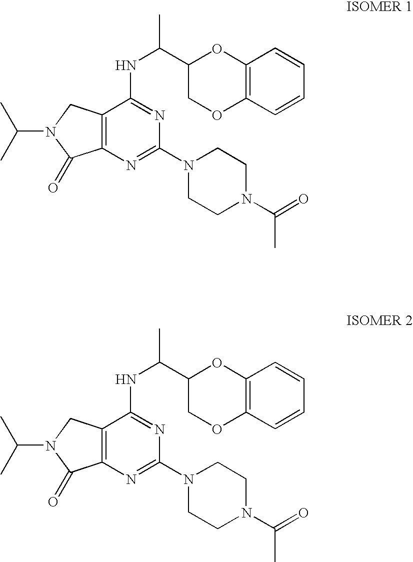

Following a procedure similar to that described in General Procedure 1 and after purification by SFC (using OD Column with MeOH+0.1% DMEA Iso at 50%), two fractions were isolated:

Fraction 1: (mixture of two diastereoisomers) yielded 40.0 mg (10.24%). HPLC: 99%; Rₜ: 1.54 minutes and Rₜ: 1.61 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CDCl₃) [symbol] ppm 1.22 (d, J=5.08 Hz, 6H), 1.43 (dd, J=24.41, 6.84 Hz, 3H), 2.10 (s, 3H), 3.41-3.52 (m, 2H), 3.63 (dd, J=8.59, 4.69 Hz, 2H), 3.69-3.91 (m, 4H), 3.92-4.16 (m, 2H), 4.19-4.32 (m, 3H), 4.52-4.63 (m, 1H), 4.72 (s, 1H), 6.76-6.88 (m, 4H). M.S. 481.2 (ESI) (MH⁺). HRMS m/z calcd for C₂₅H₃₂N₆O₄ [M+H]⁺ 481.2557, found 481.2528.

Fraction 2: (d.e. and e.e.>95%) yielded 28.0 mg (7.17%). HPLC purity: >98% (215 nm), >99% (254 nm), >97% (280 nm); Rₜ: 1.61 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.23 (d, J=6.25 Hz, 6H), 1.32-1.50 (m, 3H), 2.11 (s, 3H), 3.51 (s, 2H), 3.65 (s, 2H), 3.81 (s, 2H), 3.90 (s, 2H), 4.06 (dd, J=10.74, 6.45 Hz, 1H), 4.27 (t, J=10.35 Hz, 4H), 4.56 (dd, J=13.48, 6.45 Hz, 2H), 6.71-6.90 (m, 4H). M.S. 481.2 (ESI) (MH⁺). HRMS m/z calcd for C₂₅H₃₂N₆O₄ [M+H]⁺ 481.2557, found 481.2528.

### Example 180

### (R)-2-(4-acetylpiperazin-1-yl)-4-(1-(4-ethoxyphenyl)-2-hydroxyethylamino)-6-isopropyl-5H-pyrrolo[3,4-d]pyrimidin-7(6H)-one

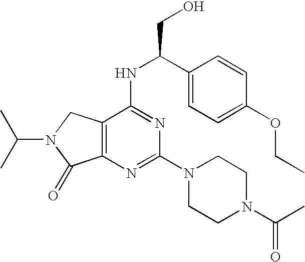

Following a procedure similar to that described in General Procedure 1, starting from (R)-2-amino-2-(4-ethoxyphenyl)ethanol and after purification by preparative LCMS (gradient 40-60% CH₃CN in H₂O containing 10 mM NH₄HCO₃) and lyophilization from CH₃CN/H₂O, the title compound was obtained as a solid (163 mg, 55.5%). HPLC purity: >99% (215 nm), >99% (254 nm), >99% (280 nm); Rₜ: 1.44 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.07-1.20 (m, 6H), 1.24 (t, J=6.84 Hz, 3H), 1.98 (s, 3 H), 3.28-3.44 (m, 4H), 3.44-3.76 (m, 4H), 3.92 (q, J=7.03 Hz, 2H), 4.00-4.22 (m, 4H), 4.33 (quin, J=6.64 Hz, 1H), 4.82 (t, J=5.66 Hz, 1H), 5.05 (d, J=5.47 Hz, 1H), 6.80 (d, J=8.59 Hz, 2H), 7.24 (d, J=8.59 Hz, 2H), 7.68 (d, J=7.42 Hz, 1H). M.S. 483.3. (ESI) (MH⁺). HRMS m/z calcd for C₂₅H₃₅N₆O₄ [M+H]⁺ 483.27143, found 483.27175.

### Example 181

### (R)-2-(4-acetylpiperazin-1-yl)-4-(1-(3-(difluoromethoxy)phenyl)ethylamino)-6-isopropyl-5H-pyrrolo[3,4-d]pyrimidin-7(6H)-one

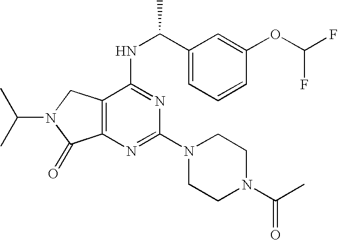

Following a procedure similar to that described in General Procedure 1, starting from (R)-1-(3-(difluoromethoxy)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (162 mg, 54.4%). HPLC purity: >99% (215 nm), >99% (254 nm), >99% (280 nm); Rₜ: 1.70 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.16 (d, J=6.64 Hz, 6H), 1.43 (d, J=7.03 Hz, 3H), 1.96 (s, 3H), 3.15-3.42 (m, 4H), 3.42-3.79 (m, 4H), 4.12 (s, 2H), 4.33 (quin, J=6.64 Hz, 1H), 5.15 (t, J=6.84 Hz, 1H), 5.72 (s, 0H), 6.91-7.04 (m, 1H), 7.17 (d, J=7.03 Hz, 1H), 7.22 (d, J=7.81 Hz, 1H), 7.26-7.40 (m, 1H), 7.85 (d, J=7.03 Hz, 1H). M.S. 489.2. (ESI) (MH⁺). HRMS m/z calcd for C₂₄H₃₀F₂N₆O₃ [M+H]⁺ 489.24202, found 489.24209.

### Example 182

### (R)-2-(4-acetylpiperazin-1-yl)-4-(1-(3-fluoro-4-(trifluoromethyl)phenyl)ethylamino)-6-isopropyl-5H-pyrrolo[3,4-d]pyrimidin-7(6H)-one

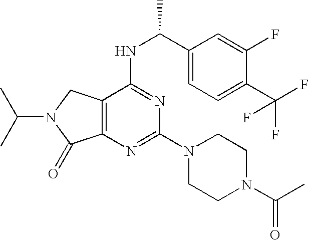

Following a procedure similar to that described in General Procedure 1, starting from (R)-1-(3-fluoro-4-(trifluoromethyl)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (141 mg, 45.5%). HPLC purity: >99% (215 nm), >99% (254 nm), >99% (280 nm); Rₜ: 1.99 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.17 (d, J=6.64 Hz, 6H), 1.45 (d, J=7.03 Hz, 3H), 1.95 (s, 3H), 3.05-3.39 (m, 4H), 3.39-3.75 (m, 4H), 4.15 (s, 2H), 4.33 (quin, J=6.64 Hz, 1H), 5.18 (t, J=6.84 Hz, 1H), 7.38 (d, J=8.20 Hz, 1H), 7.48 (d, J=12.11 Hz, 1H), 7.68 (t, J=7.81 Hz, 1H), 7.93 (d, J=6.25 Hz, 1H). M.S. 509.2. (ESI) (MH⁺). HRMS m/z calcd for C₂₄H₂₈F₄N₆O₂ [M+H]⁺ 509.22826, found 509.22907.

### Example 183

### (R)-2-(4-acetylpiperazin-1-yl)-4-(1-(2-fluoro-5-(trifluoromethyl)phenyl)ethylamino)-6-isopropyl-5H-pyrrolo[3,4-d]pyrimidin-7(6H)-one

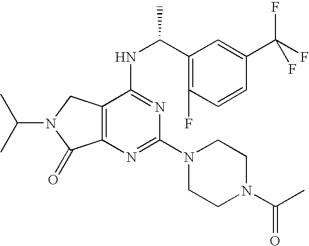

Following a procedure similar to that described in General Procedure 1, starting from (B)-1-(2-fluoro-5-(trifluoromethyl)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (188 mg, 60.6%). HPLC purity: >98% (215 nm), >97% (254 nm), >98% (280 nm); Rₜ: 1.92 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.17 (d, J=6.64 Hz, 6H), 1.48 (d, J=7.03 Hz, 3H), 1.97 (s, 3H), 3.17-3.44 (m, 4H), 3.44-3.82 (m, 4H), 4.17 (s, 2H), 4.32 (quin, J=6.74 Hz, 1H), 5.45 (t, J=6.84 Hz, 1H), 7.40 (t, J=9.18 Hz, 1H), 7.57-7.73 (m, 1H), 7.82 (d, J=4.69 Hz, 1H), 8.21 (d, J=6.64 Hz, 1H). M.S. 509.2. (ESI) (MH⁺). HRMS m/z calcd for C₂₄H₂₈F₄N₆O₂ [M+H]⁺ 509.22826, found 509.22941.

### Example 184

### (R)-2-(4-acetylpiperazin-1-yl)-4-(1-(3-fluoro-5-(trifluoromethyl)phenyl)ethylamino)-6-isopropyl-5H-pyrrolo[3,4-d]pyrimidin-7(6H)-one

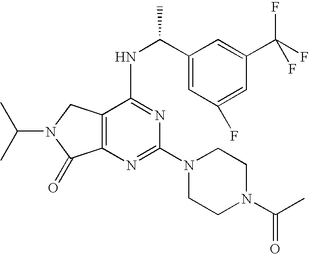

Following a procedure similar to that described in General Procedure 1, starting from (R)-1-(3-fluoro-5-(trifluoromethyl)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (198 mg, 63.8%). HPLC purity: >99% (215 nm), >99% (254 nm), >99% (280 nm); Rₜ: 1.95 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.17 (d, J=6.64 Hz, 6H), 1.45 (d, J=7.03 Hz, 3H), 1.96 (s, 3H), 3.12-3.40 (m, 4H), 3.40-3.72 (m, 4H), 4.15 (d, J=4.30 Hz, 2H), 4.33 (quin, J=6.64 Hz, 1H), 5.21 (t, J=6.84 Hz, 1H), 7.47 (d, J=8.59 Hz, 1H), 7.52 (d, J=9.38 Hz, 1H), 7.63 (s, 1H), 7.90 (d, J=6.64 Hz, 1H). M.S. 509.2. (ESI) (MH⁺). HRMS m/z calcd for C₂₄H₂₈F₄N₆O₂ [M+H]⁺ 509.22826, found 509.22874.

### Example 185

### (R)-2-(4-acetylpiperazin-1-yl)-4-(cyclopropyl(4-ethoxyphenyl)methylamino)-6-isopropyl-5H-pyrrolo[3,4-d]pyrimidin-7(6H)-one

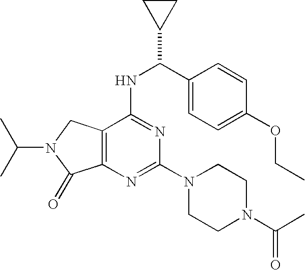

Following a procedure similar to that described in General Procedure 1, starting from (R)-cyclopropyl(4-ethoxyphenyl)methanamine, after purification by preparative LCMS (gradient 40-60% CH₃CN in H₂O containing 10 mM NH₄HCO₃) and lyophilization from CH₃CN/H₂O, the title compound was obtained as a solid (85 mg, 28.3%). HPLC purity: >97% (215 nm), >98% (254 nm), >98% (280 nm); Rₜ: 1.76 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 0.34 (d, J=2.73 Hz, 2H), 0.42-0.65 (m, 2H), 1.20 (dd, J=6.25, 3.12 Hz, 6H), 1.24-1.40 (m, 4H), 1.99 (s, 3H), 3.21-3.46 (m, 4H), 3.46-3.76 (m, 4H), 3.97 (q, J=6.77 Hz, 2H), 4.16 (br. s., 2H), 4.36 (t, J=7.03 Hz, 2H), 6.84 (d, J=8.20 Hz, 2H), 7.31 (d, J=8.59 Hz, 2H), 8.03 (d, J=7.42 Hz, 1H). M.S. 493.2. (ESI) (MH⁺). HRMS m/z calcd for C₂₇H₃₆N₆O₃ [M+H]⁺ 493.29217, found 493.29271.

### Example 186

### (R)-2-(4-acetylpiperazin-1-yl)-4-(1-(2-fluoro-4-(trifluoromethyl)phenyl)ethylamino)-6-isopropyl-5H-pyrrolo[3,4-d]pyrimidin-7(6H)-one

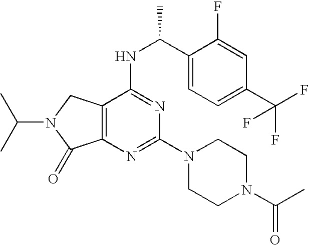

Following a procedure similar to that described in General Procedure 1, starting from (R)-1-(2-fluoro-4-(trifluoromethyl)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (153 mg, 49.3%). HPLC purity: >99% (215 nm), >99% (254 nm), >99% (280 nm); Rₜ: 1.85 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.21 (d, J=7.03 Hz, 6H), 1.51 (d, J=7.03 Hz, 3H), 1.99 (s, 3H), 3.33 (s, 4H), 3.40-3.71 (m, 4H), 4.20 (s, 2H), 4.38 (quin, J=6.74 Hz, 1H), 5.42 (t, J=6.64 Hz, 1H), 7.54 (d, J=7.81 Hz, 1H), 7.59-7.74 (m, 2H), 8.05 (d, J=6.64 Hz, 1H). M.S. 509.2. (ESI) (MH⁺). HRMS m/z calcd for C₂₄H₂₈F₄N₆O₂ [M+H]⁺ 509.22826, found 509.22898.

### Example 187

### (R)-2-(4-acetylpiperazin-1-yl)-4-(1-(2-fluoro-3-(trifluoromethyl)phenyl)ethylamino)-6-isopropyl-5H-pyrrolo[3,4-d]pyrimidin-7(6H)-one

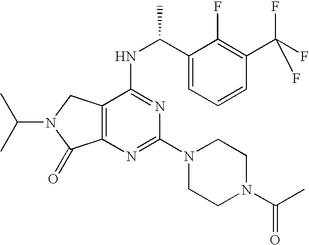

Following a procedure similar to that described in General Procedure 1, starting from (R)-1-(2-fluoro-3-(trifluoromethyl)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (149 mg, 48.0%). HPLC purity: >98% (215 nm), >99% (254 nm), >99% (280 nm); Rₜ: 1.83 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.21 (d, J=6.64 Hz, 6H), 1.51 (d, J=7.03 Hz, 3H), 1.99 (s, 3H), 3.05-3.82 (m, 8H), 4.20 (s, 2H), 4.38 (quin, J=6.74 Hz, 1H), 5.43 (t, J=6.64 Hz, 1H), 7.35 (t, J=7.81 Hz, 1H), 7.63 (t, J=6.84 Hz, 1H), 7.74 (t, J=7.03 Hz, 1H), 8.06 (d, J=6.64 Hz, 1H). M.S. 509.2. (ESI) (MH⁺). HRMS m/z calcd for C₂₄H₂₈F₄N₆O₂ [M+H]⁺ 509.22826, found 509.22851.

### Example 188

### (R)-2-(4-acetylpiperazin-1-yl)-4-(1-(4-fluoro-3-(trifluoromethyl)phenyl)ethylamino)-6-isopropyl-5H-pyrrolo[3,4-d]pyrimidin-7(6H)-one

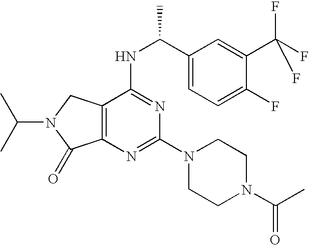

Following a procedure similar to that described in General Procedure 1, starting from (R)-1-(4-fluoro-3-(trifluoromethyl)phenyl)ethanamine, after purification by preparative LCMS (gradient 40-60% CH₃CN in H₂O containing 10 mM NH₄HCO₃) and lyophilization from CH₃CN/H₂O, the title compound was obtained as a solid (192 mg, 61.9%). HPLC purity: >99% (215 nm), >99% (254 nm), >99% (280 nm); Rₜ: 1.82 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.64 Hz, 6H), 1.49 (d, J=7.03 Hz, 3H), 2.01 (s, 3H), 3.14-3.46 (m, 4H), 3.46-3.82 (m, 4H), 4.17 (s, 2H), 4.37 (quin, J=6.64 Hz, 1H), 5.25 (t, J=6.84 Hz, 1H), 7.35-7.54 (m, 1H), 7.69-7.79 (m, 1H), 7.82 (d, J=6.64 Hz, 1H), 7.93 (d, J=7.03 Hz, 1H). M.S. 509.2. (ESI) (MH⁺). HRMS m/z calcd for C₂₄H₂₈F₄N₆O₂ [M+H]⁺ 509.22826, found 509.22805.

### Example 189

### (S)-2-(4-acetylpiperazin-1-yl)-4-(1-(4-ethoxyphenyl)-2-hydroxyethylamino)-6-isopropyl-5H-pyrrolo[3,4-d]pyrimidin-7(6H)-one

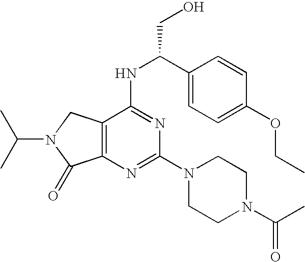

Following a procedure similar to that described in General Procedure 1, starting from (S)-2-amino-2-(4-ethoxyphenyl)ethanol, after purification by preparative LCMS (gradient 30-50% CH₃CN in H₂O containing 10 mM NH₄HCO₃) and lyophilization from CH₃CN/H₂O, the title compound was obtained as a solid (204 mg, 69.3%). HPLC purity: >99% (215 nm), >99% (254 nm), >99% (280 nm); Rₜ: 1.37 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.08-1.25 (m, 6H), 1.29 (t, J=6.84 Hz, 3H), 2.02 (s, 3H), 3.23-3.49 (m, 4H), 3.51-3.79 (m, 6H), 3.97 (q, J=7.03 Hz, 2H), 4.16 (br. s., 2H), 4.37 (quin, J=6.64 Hz, 1H), 4.87 (t, J=5.47 Hz, 1H), 5.10 (d, J=5.86 Hz, 1H), 6.84 (d, J=8.59 Hz, 2H), 7.28 (d, J=8.59 Hz, 2H), 7.72 (d, J=7.42 Hz, 1H). M.S. 483.3. (ESI) (MH⁺). HRMS m/z calcd for C₂₅H₃₄N₆O₄ [M+H]⁺ 483.27143, found 483.27127.

### Example 190

### (R)-2-(4-acetylpiperazin-1-yl)-4-(1-(3-fluoro-4-isopropoxyphenyl)ethylamino)-6-isopropyl-5H-pyrrolo[3,4-d]pyrimidin-7(6H)-one

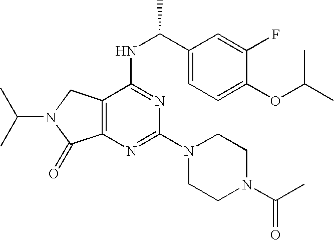

Following a procedure similar to that described in General Procedure 1, starting from (R)-1-(3-fluoro-4-isopropoxyphenyl)ethanamine hydrochloride (Intermediate 48) and after purification by preparative LCMS (gradient 40-60% CH₃CN in H₂O containing 10 mM NH₄HCO₃) and lyophilization from CH₃CN/H₂O, the title compound was obtained as a solid (158 mg, 51.9%). HPLC purity: >99% (215 nm), >99% (254 nm), >98% (280 nm); Rₜ: 1.79 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.64 Hz, 6H), 1.24 (d, J=5.86 Hz, 6H), 1.45 (d, J=7.03 Hz, 3H), 2.01 (s, 3H), 3.18-3.49 (m, 4H), 3.49-3.84 (m, 4H), 4.15 (s, 2H), 4.37 (quin, J=6.64 Hz, 1H), 4.54 (quin, J=6.05 Hz, 1H), 5.16 (quin, J=6.71, 6.45 Hz, 1H), 7.00-7.16 (m, 2H), 7.21 (d, J=12.50 Hz, 1H), 7.79 (d, J=7.03 Hz, 1H). M.S. 499.2. (ESI) (MH⁺). HRMS m/z calcd for C₂₆H₃₅FN₆O₃ [M+H]⁺ 499.28274, found 499.28243.

### Example 191

### (R)-2-(4-acetylpiperazin-1-yl)-4-(1-(4-cyclobutoxyphenyl)ethylamino)-6-isopropyl-5H-pyrrolo[3,4-d]pyrimidin-7(6H)-one

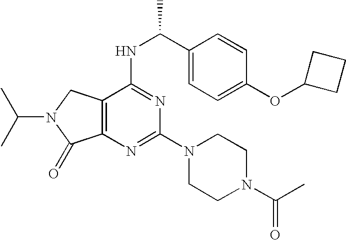

Following a procedure similar to that described in General Procedure 1, starting from (R)-1-(4-cyclobutoxyphenyl)ethanamine hydrochloride (Intermediate 49) and after purification by preparative LCMS (gradient 40-60% CH₃CN in H₂O containing 10 mM NH₄HCO₃) and lyophilization from CH₃CN/H₂O, the title compound was obtained as a solid (84 mg, 53.3%). [α]_{D}=+153.1 (c=0.01, MeOH). HPLC purity: >99% (215 nm), >99% (254 nm), >98% (280 nm); Rₜ: 1.86 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.19 (d, J=6.64 Hz, 6H), 1.44 (d, J=7.03 Hz, 3H), 1.51-1.82 (m, 2H), 1.91-2.01 (m, 2H), 2.02 (s, 3H), 2.26-2.46 (m, 2H), 3.22-3.51 (m, 4H), 3.52-3.84 (m, 4H), 4.13 (s, 2H), 4.36 (quin, J=6.74 Hz, 1H), 4.55-4.75 (m, 1H), 5.16 (t, J=7.03 Hz, 1H), 6.76 (d, J=8.59 Hz, 2H), 7.28 (d, J=8.59 Hz, 2H), 7.77 (d, J=7.42 Hz, 1H). M.S. 493.2. (ESI) (MH⁺). HRMS m/z calcd for C₂₇H₃₆N₆O₃ [M+H]⁺ 493.29217, found 493.29133.

### Example 192

### (R)-2-(4-acetylpiperazin-1-yl)-4-(1-(4-cyclopropylphenyl)ethylamino)-6-isopropyl-5H-pyrrolo[3,4-d]pyrimidin-7(6H)-one

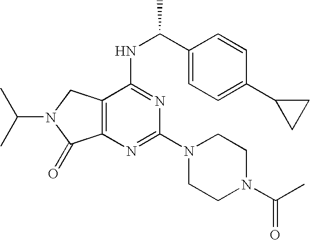

Following a procedure similar to that described in General Procedure 1, starting from (R)-1-(4-cyclopropylphenyl)ethanamine hydrochloride (Intermediate 50) and after purification by preparative LCMS (gradient 40-60% CH₃CN in H₂O containing 10 mM NH₄HCO₃) and lyophilization from CH₃CN/H₂O, the title compound was obtained as a solid (203 mg, 71.9%). [α]_{D}=+160.8 (c=0.01, MeOH). HPLC purity: >98% (215 nm), >99% (254 nm), >98% (280 nm); Rₜ: 1.78 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 0.48-0.71 (m, 2H), 0.78-1.04 (m, 2H), 1.19 (d, J=7.03 Hz, 6H), 1.45 (d, J=6.64 Hz, 3H), 1.77-1.95 (m, 1H), 2.02 (s, 3H), 3.17-3.47 (m, 4H), 3.49-3.83 (m, 4H), 4.14 (s, 2H), 4.36 (quin, J=6.64 Hz, 1H), 5.16 (quin, J=6.84 Hz, 1H), 6.99 (d, J=8.20 Hz, 2H), 7.25 (d, J=8.20 Hz, 2H), 7.80 (d, J=7.42 Hz, 1H). M.S. 463.3. (ESI) (MH⁺). HRMS m/z calcd for C₂₆H₃₄N₆O₂ [M+H]⁺ 463.28160, found 463.28129.

### Example 193

### (R)-2-(4-acetylpiperazin-1-yl)-4-(1-(3-fluoro-4-propoxyphenyl)ethylamino)-6-isopropyl-5H-pyrrolo[3,4-d]pyrimidin-7(6H)-one

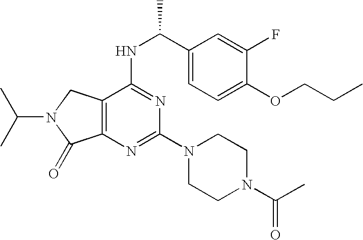

Following a procedure similar to that described in General Procedure 1, starting from (R)-1-(3-fluoro-4-propoxyphenyl)ethanamine hydrochloride (Intermediate 51) and after purification by preparative LCMS (gradient 40-60% CH₃CN in H₂O containing 10 mM NH₄HCO₃) and lyophilization from CH₃CN/H₂O, the title compound was obtained as a solid (200 mg, 65.8%). [α]_{D}=+120.3 (c=0.01, MeOH). HPLC purity: >99% (215 nm), >99% (254 nm), >98% (280 nm); Rₜ: 1.86 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 0.95 (t, J=7.42 Hz, 3H), 1.20 (d, J=6.64 Hz, 6H), 1.44 (d, J=6.64 Hz, 3H), 1.70 (sxt, J=7.03 Hz, 2H), 2.02 (s, 3H), 3.25-3.48 (m, 4H), 3.52-3.79 (m, 4H), 3.95 (t, J=6.64 Hz, 2H), 4.15 (s, 2H), 4.36 (quin, J=6.64 Hz, 1H), 5.16 (t, J=7.03 Hz, 1H), 6.99-7.17 (m, 2H), 7.22 (d, J=12.89 Hz, 1H), 7.79 (d, J=7.42 Hz, 1H). M.S. 499.2. (ESI) (MH⁺). HRMS m/z calcd for C₂₆H₃₅FN₆O₃ [M+H]⁺ 499.28274, found 499.28249.

### Example 194

### (R)-2-(4-acetylpiperazin-1-yl)-4-(1-(5-chloro-6-ethoxypyridin-3-yl)ethylamino)-6-isopropyl-5H-pyrrolo[3,4-d]pyrimidin-7(6H)-one

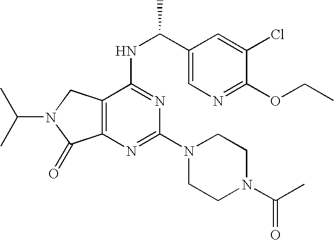

Following a procedure similar to that described in General Procedure 1, starting from (R)-1-(5-chloro-6-ethoxypyridin-3-yl)ethanamine hydrochloride (Intermediate 52) and after purification by preparative LCMS (gradient 40-60% CH₃CN in H₂O containing 10 mM NH₄HCO₃) and lyophilization from CH₃CN/H₂O, the title compound was obtained as a solid (194 mg, 63.4%). [α]_{D}=+144.3 (c=0.01, MeOH). HPLC purity: >98% (215 nm), >98% (254 nm), >98% (280 nm); Rₜ: 1.72 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.20 (d, J=6.64 Hz, 6H), 1.31 (t, J=7.03 Hz, 3H), 1.49 (d, J=7.03 Hz, 3H), 2.02 (s, 3H), 3.21-3.51 (m, 4H), 3.51-3.84 (m, 4H), 4.16 (d, J=3.52 Hz, 2H), 4.35 (q, J=7.03 Hz, 3H), 5.20 (t, J=7.03 Hz, 1H), 7.82 (d, J=7.03 Hz, 1H), 7.90 (d, J=1.95 Hz, 1H), 8.15 (d, J=1.95 Hz, 1H). M.S. 502.2. (ESI) (MH⁺). HRMS m/z calcd for C₂₄H₃₃ClN₇O₃ [M+H]⁺ 502.23279, found 502.23330.

### Example 195

### 2-(4-acetylpiperazin-1-yl)-6-isopropyl-4-(1-(2-methoxyphenyl)-2-methylpropan-2-ylamino)-5H-pyrrolo[3,4-d]pyrimidin-7(6H)-one

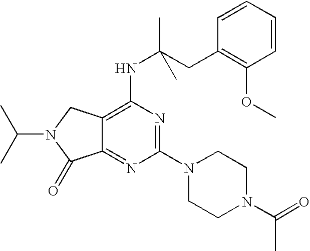

Following the General Procedure 2, starting from 2-chloro-6-isopropyl-4-(1-(2-methoxyphenyl)-2-methylpropan-2-ylamino)-5H-pyrrolo[3,4-d]pyrimidin-7(6H)-one (Intermediate 88) and after purification by silica gel chromatography (0-10% MeOH in DCE) followed by preparative HPLC (gradient 55-75% CH₃CN in H₂O containing 10 mM NH₄HCO₃), the title compound (27.0 mg, 8.74%) was obtained as a solid. Purity: >99% (215 nm), >99% (254 nm), >99% (280 nm); Rₜ: 1.90 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.26 (d, J=7.03 Hz, 6H), 1.57 (s, 6H), 2.13 (s, 3H), 2.99 (s, 2H), 3.47-3.52 (m, 2H), 3.64-3.70 (m, 2H), 3.82-3.87 (m, 2H), 3.89-3.94 (m, 4H), 3.96 (s, 3H), 4.67 (quin, J=6.64 Hz, 1H), 5.80 (s, 1H), 6.93-7.00 (m, 2H), 7.09-7.15 (m, 1H), 7.24-7.31 (m, 1H). MS [M+H]⁺ 481.2 (ESI). HRMS m/z calcd for C₂₆H₃₇N₆O₃ [M+H]⁺ 481.29217, found 481.29197.

### Example 196

### 2-(4-acetylpiperazin-1-yl)-6-isopropyl-4-(1-(4-methoxyphenyl)-2-methylpropan-2-ylamino)-5H-pyrrolo[3,4-d]pyrimidin-7(6H)-one

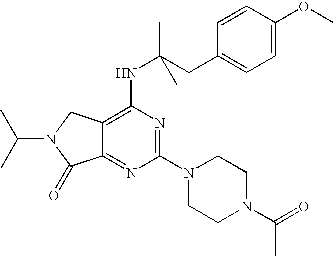

Following the General Procedure 2, starting from 2-chloro-6-isopropyl-4-(1-(4-methoxyphenyl)-2-methylpropan-2-ylamino)-5H-pyrrolo[3,4-d]pyrimidin-7(6H)-one (Intermediate 89) and after purification by silica gel chromatography (0-10% MeOH in DCM) followed by preparative HPLC (gradient 45-65% CH₃CN in H₂O containing 10 mM NH₄HCO₃), the title compound (34.0 mg, 18.34%) was obtained as a solid. Purity: >99% (215 nm), >98% (254 nm), >99% (280 nm); Rₜ: 1.73 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.24 (d, J=6.64 Hz, 6H), 1.47 (s, 6H), 2.15 (s, 3H), 3.17 (s, 2H), 3.48-3.59 (m, 2H), 3.65-3.74 (m, 2H), 3.78 (s, 3H), 3.86-3.94 (m, 4H), 3.94-4.00 (m, 2H), 4.67 (qt, 1H), 6.75-6.81 (m, 2H), 6.93 (d, 2H). MS [M+H]⁺ 481.2 (ESI). HRMS m/z calcd for C₂₆H₃₇N₆O₃ [M+H]⁺ 481.29217, found 481.29199.

### Example 197

### 2-(4-acetylpiperazin-1-yl)-6-isopropyl-4-(2-methyl-1-o-tolylpropan-2-ylamino)-5H-pyrrolo[3,4-d]pyrimidin-7(6H)-one

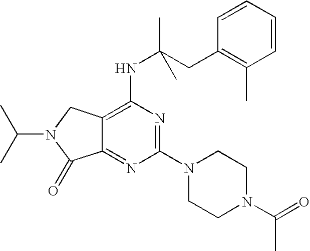

Following the General Procedure 2, starting from 2-chloro-6-isopropyl-4-(2-methyl-1-o-tolylpropan-2-ylamino)-5H-pyrrolo[3,4-d]pyrimidin-7(6H)-one (Intermediate 90) and after purification by silica gel chromatography (0-10% MeOH in DCM) followed by preparative HPLC(X-Bridge Prep C18 OBD, 30×50 mm, 5 μm particle size, gradient 45-65% CH₃CN in H₂O containing 10 mM NH₄HCO₃), the title compound (14.0 mg, 10.2%) was obtained as a solid. Purity: >95% (215 nm), >97% (254 nm), >98% (280 nm); Rₜ: 1.86 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.25 (d, J=7.03 Hz, 6H), 1.50 (s, 6H), 2.14 (s, 3H), 2.34 (s, 3H), 3.27 (s, 2H), 3.49-3.57 (m, 2H), 3.65-3.73 (m, 2H), 3.86-3.92 (m, 2H), 3.94 (s, 2H), 3.94-4.00 (m, 2H), 4.17 (s, 1H), 4.68 (quin, J=6.74 Hz, 1H), 6.90 (d, J=7.81 Hz, 1H), 7.01-7.09 (m, 1H), 7.10-7.21 (m, 2H). MS [M+H]⁺ 465.2 (ESI). HRMS m/z calcd for C₂₆H₃₇N₆O₂ [M+H]⁺ 465.29725, found 465.29693.

### Example 198

### 2-(4-acetylpiperazin-1-yl)-4-(1-(4-ethoxyphenyl)-2-methylpropan-2-ylamino)-6-isopropyl-5H-pyrrolo[3,4-d]pyrimidin-7(6H)-one

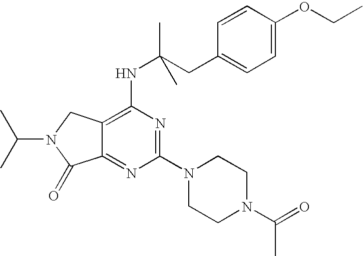

Following the General Procedure 2, starting from 2-chloro-4-(1-(4-ethoxyphenyl)-2-methylpropan-2-ylamino)-6-isopropyl-5H-pyrrolo[3,4-d]pyrimidin-7(6H)-one (Intermediate 91) and after purification by silica gel chromatography (0-10% MeOH in DCM) followed by preparative HPLC (high pH: Phenomenex Gemini C18, 21.2×250 mm, 5 μm particle size, gradient 45-65% CH₃CN in H₂O containing 10 mM NH₄HCO₃) to give the title compound (27 mg, 10%) as a solid. Purity: >96% (215 nm), >96% (254 nm), >95% (280 nm); Rₜ: 1.90 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.24 (d, J=6.64 Hz, 6H), 1.40 (t, J=7.03 Hz, 3H), 1.47 (s, 6H), 2.15 (s, 3H), 2.17 (s, 2H), 3.15 (s, 2H), 3.51-3.56 (m, 2H), 3.68-3.73 (m, 2H), 3.88-3.92 (m, 2H), 3.95-4.02 (m, 4H), 4.07 (s, 1H), 4.62-4.72 (m, 1H), 6.75-6.80 (m, 2H), 6.89-6.94 (m, 2H). MS [M+H]⁺ 495.2 (ESI). HRMS m/z calcd for C₂₇H₃₉N₆O₃ [M+H]⁺ 495.30782, found 495.30734.

### Example 199

### 2-(4-acetylpiperazin-1-yl)-4-(benzo[d]thiazol-2-ylmethylamino)-6-isopropyl-5H-pyrrolo[3,4-d]pyrimidin-7(6H)-one

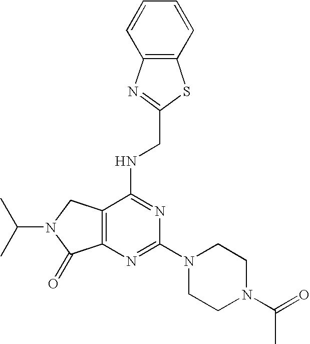

Following the General Procedure 1 and after purification by silica gel chromatography (0-10% MeOH in DCM) followed by preparative HPLC (gradient 45-65% CH₃CN in H₂O containing 10 mM NH₄HCO₃), the title compound (0.142 g, 50.0%) was obtained as a solid. Purity: >99% (215 nm), >99% (254 nm), >99% (280 nm); Rₜ: 1.40 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.26 (d, J=7.03 Hz, 6H), 2.09 (s, 3H), 3.35-3.41 (m, 2H), 3.56-3.62 (m, 2H), 3.80-3.89 (m, 4H), 4.18 (s, 2H), 4.67 (quin, J=6.74 Hz, 1H), 5.09 (d, J=5.47 Hz, 2H), 6.07 (t, J=5.47 Hz, 1H), 7.39 (ddd, J=8.11, 7.13, 1.17 Hz, 1H), 7.49 (ddd, J=8.30, 7.13, 1.37 Hz, 1H), 7.83-7.88 (m, 1H), 7.98 (d, J=7.81 Hz, 1H). MS [M+H]⁺ 466.2 (ESI). HRMS m/z calcd for C₂₃H₂₈N₇O₂S[M+H]⁺ 466.20197, found 466.20189.

### Example 200

### 6-isopropyl-4-((isoquinolin-3-ylmethyl)(methyl)amino)-2-(4-methyl-3-oxopiperazin-1-yl)-5H-pyrrolo[3,4-d]pyrimidin-7(6H)-one

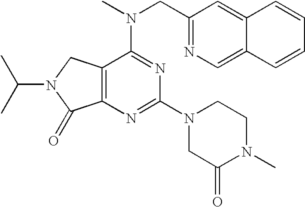

Following a procedure similar to that described in General Procedure 1, starting from 1-(isoquinolin-3-yl)-N-methylmethanamine (Intermediate 70) and after purification by silica gel chromatography (0-10% MeOH in DCM) followed by preparative HPLC (high pH: Phenomenex Gemini C18, 21.2×250 mm, 5 μm particle size, gradient 45-65% CH₃CN in H₂O containing 10 mM NH₄HCO₃), the title compound (15 mg, 7.12%) was obtained as a solid. Purity: >99% (215 nm), >98% (254 nm), >98% (280 nm); Rₜ: 1.26 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.20 (d, J=6.64 Hz, 6H), 3.01 (s, 3H), 3.35 (s, 3H), 3.39 (t, J=5.27 Hz, 2H), 4.16 (t, J=5.27 Hz, 2H), 4.38 (s, 2H), 4.43 (s, 2H), 4.63 (qt, 1H), 5.03 (s, 2H), 7.50 (s, 1H), 7.58-7.64 (m, 1H), 7.67-7.74 (m, 1H), 7.77-7.82 (m, 1H), 7.98 (d, J=8.20 Hz, 1H), 9.24 (s, 1H). MS [M+H] 460.2 (ESI). HRMS m/z calcd for C₂₅H₂₉N₇O₂[M+H]⁺ 460.24555, found 460.24568.

### Example 201

### (R)-4-(1-(4-ethoxy-3-fluorophenyl)ethylamino)-6-isopropyl-2-(4-methyl-3-oxopiperazin-1-yl)-5H-pyrrolo[3,4-d]pyrimidin-7(6H)-one

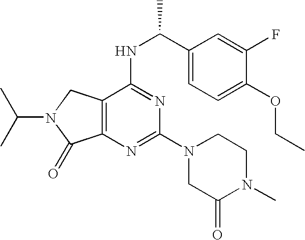

Following a procedure similar to that described in General Procedure 1, starting from (R)-1-(4-ethoxy-3-fluorophenyl)ethanamine hydrochloride (Intermediate 40) and after purification by silica gel chromatography (0-10% MeOH in DCM) followed by preparative HPLC (gradient 45-65% CH₃CN in H₂O containing 10 mM NH₄HCO₃), the title compound (0.035 g, 16.53%) was obtained as a solid. [α]_{D}=+113.7 (c=0.008, MeOH). HPLC purity: >97% (215 nm), >98% (254 nm), >98% (280 nm); Rₜ: 1.73 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.25 (d, J=7.03 Hz, 6H), 1.44 (t, J=7.03 Hz, 3H), 1.56 (d, J=7.03 Hz, 3H), 3.01 (s, 3H), 3.37 (t, J=5.27 Hz, 2H), 4.05-4.18 (m, 6H), 4.26-4.34 (m, 1H), 4.40-4.48 (m, 1H), 4.63-4.71 (m, 1H), 4.73 (d, J=7.03 Hz, 1H), 5.32 (t, J=6.64 Hz, 1H), 6.93 (t, J=8.20 Hz, 1H), 7.05-7.08 (m, 1H), 7.09 (s, 1H). M.S. (found): 471.3 (ESI) (MH⁺). HRMS m/z calcd for C₂₄H₃₂FN₆O₃[M+H]⁺ 471.25144, found 471.25109.

### Example 202

### (R)-2-(4-acetylpiperazin-1-yl)-4-(1-(4-ethoxy-2-methoxyphenyl)ethylamino)-6-isopropyl-5H-pyrrolo[3,4-d]pyrimidin-7(6H)-one

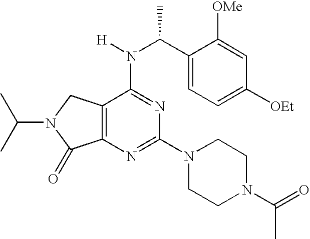

Following a procedure similar to that described in General Procedure 1, starting from (R)-1-(4-ethoxy-2-methoxyphenyl)ethanamine hydrochloride (Intermediate 55) and after purification by silica gel chromatography (0-10% MeOH/DCM) followed by preparative HPLC (high pH: Phenomenex Gemini C18, 21.2×250 mm, 5 μm particle size, gradient 45-65% CH₃CN in H₂O containing 10 mM NH₄HCO₃), the title compound (0.182 g, 52.4%) was obtained as a solid. [α]_{D}=+75.5 (c=0.01, MeOH). HPLC purity: >99% (215 nm), >99% (254 nm), >99% (280 nm); Rₜ: 1.73 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.16-1.31 (m, 6H), 1.41 (t, J=7.03 Hz, 3H), 1.53 (d, J=7.03 Hz, 3H), 2.14 (s, 3H), 3.45 (t, J=5.08 Hz, 2H), 3.56-3.69 (m, 2H), 3.78-3.84 (m, 2H), 3.87 (s, 3H), 3.87-3.94 (m, 2H), 3.97-4.07 (m, 4H), 4.65 (qt, 1H), 5.19 (d, 1H), 5.40 (br. s., 1H), 6.43 (dd, J=8.59, 2.34 Hz, 1H), 6.49 (d, J=2.34 Hz, 1H), 7.13 (d, 1H). M.S. 497.2 (ESI) (MH⁺). HRMS m/z calcd for C₂₆H₃₇N₆O₄[M+H]⁺ 497.28708, found 497.28681.

### Example 203

### (R)-2-(4-acetylpiperazin-1-yl)-4-(1-(4-isopropoxy-2-methoxyphenyl)ethylamino)-6-isopropyl-5H-pyrrolo[3,4-d]pyrimidin-7(6H)-one

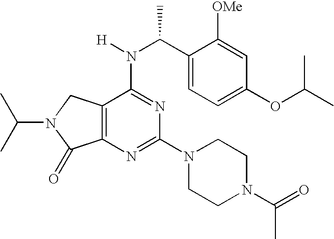

Following a procedure similar to that described in General Procedure 1, starting from (R)-1-(4-isopropoxy-2-methoxyphenyl)ethanamine hydrochloride (Intermediate 56) and after purification by silica gel chromatography (0-10% MeOH/DCM) followed by preparative HPLC purification (high pH: Phenomenex Gemini C18, 21.2×250 mm, 5 μm particle size, gradient 45-65% CH₃CN in H₂O containing 10 mM NH₄HCO₃), the title compound (0.114 g, 27.9%) was obtained as a solid. [α]_{D}=+102.7 (c=0.01, MeOH). HPLC purity: >99% (215 nm), >99% (254 nm), >99% (280 nm); Rₜ: 1.84 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.20-1.27 (m, 6H), 1.33 (d, J=6.25 Hz, 6H), 1.53 (d, J=6.64 Hz, 3H), 2.14 (s, 3H), 3.45 (t, J=5.27 Hz, 2H), 3.56-3.70 (m, 2H), 3.78-3.84 (m, 2H), 3.86 (s, 3H), 3.90 (qd, 2H), 3.98-4.09 (m, 2H), 4.52 (qt, 1H), 4.65 (qt, 1H), 5.21 (d, J=7.42 Hz, 1H), 5.40 (br. s., 1H), 6.42 (dd, J=8.40, 2.15 Hz, 1H), 6.47 (d, J=2.34 Hz, 1H), 7.12 (d, 1H). M.S. 511.2 (ESI) (MH⁺). HRMS m/z calcd for C₂₇H₉N₆O₄[M+H]⁺ 511.30273, found 511.30183.

### Example 204

### (R)-2-(4-acetylpiperazin-1-yl)-4-(1-(4-ethoxy-2-fluorophenyl)ethylamino)-6-isopropyl-5H-pyrrolo[3,4-d]pyrimidin-7(6H)-one

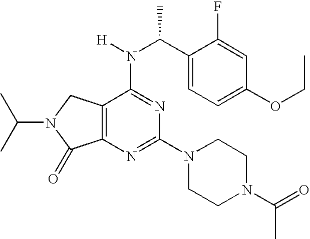

Following a procedure similar to that described in General Procedure 1, starting from (R)-1-(4-ethoxy-2-fluorophenyl)ethanamine hydrochloride (Intermediate 57) and after purification by silica gel chromatography (0-10% MeOH/DCM) followed by preparative HPLC (high pH: Phenomenex Gemini C18, 21.2×250 mm, 5 μm particle size, gradient 45-65% CH₃CN in H₂O containing 10 mM NH₄HCO₃, retention time 14.64 min), the title compound (0.111 g, 45.8%) was obtained as a solid. [α]_{D}=+120.1 (c=0.01, MeOH). HPLC purity: >99% (215 nm), >99% (254 nm), >99% (280 nm); Rₜ: 1.72 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.26 (d, J=6.64 Hz, 6H), 1.40 (t, J=7.03 Hz, 3H), 1.58 (d, J=7.03 Hz, 3H), 2.13 (s, 3H), 3.43 (t, J=5.27 Hz, 2H), 3.52-3.68 (m, 2H), 3.77-3.92 (m, 4H), 3.99 (q, J=7.03 Hz, 2H), 4.09 (s, 2H), 4.67 (quin, J=7.03, 6.77 Hz, 1H), 4.97 (d, 1H), 5.41 (quin, J=6.93 Hz, 1H), 6.56-6.66 (m, 2H), 7.19 (t, 1H). M.S. (found): 485.2 (ESI) (MH⁺). HRMS m/z calcd for C₂₅H₃₄FN₆O₃ [M+H]⁺ 485.26709, found 485.26668.

### Example 205

### 2-(4-acetylpiperazin-1-yl)-4-(isochoman-1-ylmethylamino)-6-isopropyl-5H-pyrrolo[3,4-d]pyrimidin-7(6H)-one

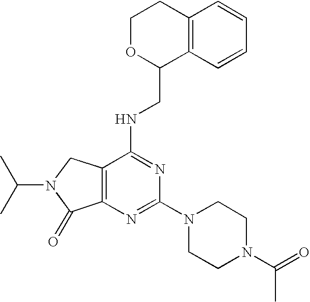

Following a procedure similar to that described in General Procedure 1 and after purification by preparative LCMS (high pH), the title compound (95 mg, 50.2%) was obtained as a solid. HPLC purity: >99% (215 nm), >99% (254 nm), >99% (280 nm); Rₜ: 1.53 minutes; Conditions: Column: Zorbax C-18, 30×4.6 mm, 1.8 u, Gradient: 5-95% B in 4.5 min, flow rate 3.5 mL/min, 70° C., A: 0.05% TFA in H₂O, B: 0.05% TFA in CH₃CN. ¹H NMR (400 MHz, CDCl₃) δ ppm 1.24 (dd, J=6.64, 3.52 Hz, 6H), 2.12 (s, 3H), 2.75 (d, J=16.02 Hz, 1H), 2.89-3.01 (m, 1H), 3.52 (s, 2H), 3.60-3.71 (m, 3H), 3.75-3.83 (m, 2H), 3.87 (s, 2H), 3.95 (s, 2H), 4.09-4.27 (m, 4H), 4.58 (s, 1H), 5.01 (d, J=8.59 Hz, 1H), 7.11-7.16 (m, 2H), 7.17-7.23 (m, 2H). M.S. 465.2 (ESI) (MH⁺).

### Example 206

### 6-isopropyl-2-(3-oxopiperazin-1-yl)-4-(quinolin-3-ylmethylamino)-5H-pyrrolo[3,4-d]pyrimidin-7(6H)-one

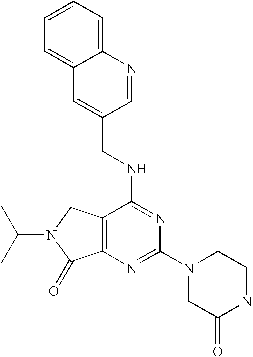

Following a procedure similar to that described in General Procedure 5 and after purification by preparative LCMS (high pH), the title compound was obtained as a solid (43.0 mg, 28.9%) following lyophilization from CH₃CN/H₂O. HPLC purity: >98% (215 nm), >97% (254 nm), >99% (280 nm); Rₜ: 0.859 minutes; Conditions: Zorbax SB C-18; Gradient: 05-95% B in 4.5 min, 70° C. A: 0.05% TFA in H₂O, B: 0.05% TFA in CH₃CN. ¹H NMR (DMSO-d₆) δ ppm 1.19 (d, J=7.03 Hz, 6H), 3.13 (s, 2H), 3.80-3.86 (m, 2H), 4.15 (d, J=6.25 Hz, 4H), 4.31-4.40 (m, 1H), 4.79 (d, J=5.47 Hz, 2H), 7.55-7.61 (m, 1H), 7.68-7.74 (m, 1H), 7.93-8.02 (m, 3H), 8.20 (t, J=5.66 Hz, 1H), 8.27 (d, J=1.56 Hz, 1H), 8.94 (d, J=1.95 Hz, 1H). M.S. 432.2 (ESI) (MH⁺). HRMS m/z calcd for C₂₃H₂₆N₇O₂ [M+H]⁺ 432.2143, found 432.2143.

### Example 207

### N-(1-(6-isopropyl-7-oxo-4-(quinolin-3-ylmethylamino)-6,7-dihydro-5H-pyrrolo[3,4-d]pyrimidin-2-yl)pyrrolidin-3-yl)acetamide

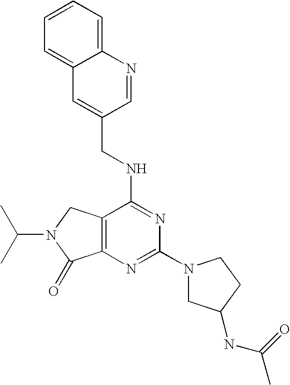

Following a procedure similar to that described in General Procedure 5 and after purification by preparative LCMS (high pH), the title compound was obtained as a solid (45.0 mg, 36.0%) following lyophilization from CH₃CN/H₂O. HPLC purity: >99% (215 nm), >99% (254 nm), >99% (280 nm); Rₜ: 0.874 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD) δ ppm 1.30 (d, J=7.03 Hz, 6H), 1.86-1.96 (m, 1H), 1.90 (s, 3H), 2.12-2.22 (m, 1H), 3.42 (dd, J=11.91, 4.49 Hz, 1H), 3.62 (t, 2H), 3.73-3.81 (m, 1H), 4.23 (s, 2H), 4.32-4.43 (m, 1H), 4.47-4.56 (m, 1H), 4.88 (s, 2H), 7.56-7.63 (m, 1H), 7.70-7.77 (m, 1H), 7.91 (d, J=7.42 Hz, 1H), 8.00 (d, J=8.20 Hz, 1H), 8.32 (d, J=0.78 Hz, 1H), 8.92 (d, J=1.95 Hz, 1H). M.S. 460.2 (ESI) (MH⁺). HRMS m/z calcd for C₂₅H₃₀N₇O₂ [M+H]⁺ 460.2455, found 460.2458.

### Example 208

### N-(1-(6-isopropyl-7-oxo-4-(quinolin-3-ylmethylamino)-6,7-dihydro-5H-pyrrolo[3,4-d]pyrimidin-2-yl)pyrrolidin-3-yl)-N-methylacetamide

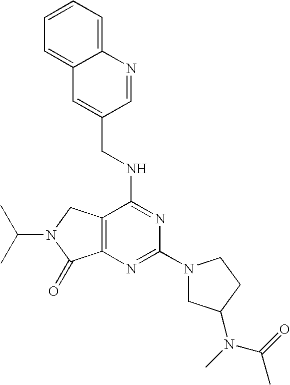

Following a procedure similar to that described in General Procedure 5 and after purification by preparative LCMS (high pH), the title compound was obtained as a solid (43.0 mg, 33.4%) following lyophilization from CH₃CN/H₂O. HPLC purity: >97% (215 nm), >97% (254 nm), >99% (280 nm); Rₜ: 0.967 minutes; Conditions: Zorbax SB C-18; Gradient: 05-95% B in 4.5 min, 70° C. A: 0.05% TFA in H₂O, B: 0.05% TFA in CH₃CN. ¹H NMR (DMSO-d₆) δ ppm 1.16 (d, J=6.64 Hz, 6H), 1.32 (d, J=7.03 Hz, 1H), 1.95-1.98 (m, 3H), 2.04 (s, 2H), 2.64 (s, 1H), 2.75 (s, 1H), 3.49-3.72 (m, 2H), 4.12 (s, 2H), 4.28-4.37 (m, 1H), 4.48-4.55 (m, 1H), 4.75 (d, J=5.47 Hz, 3H), 4.97-5.08 (m, 1H), 7.56 (t, J=7.62 Hz, 1H), 7.69 (t, J=7.62 Hz, 1H), 7.87-7.94 (m, 1H), 7.96 (d, J=8.20 Hz, 1H), 8.10 (s, 1H), 8.25 (s, 1H), 8.92 (s, 1H). M.S. 474.2 (ESI) (MH⁺). HRMS m/z calcd for C₂₆H₃₂N₇O₂ [M+H]⁺ 474.2612, found 474.2612.

### Example 209

### 1-(6-isopropyl-7-oxo-4-(quinolin-3-ylmethylamino)-6,7-dihydro-5H-pyrrolo[3,4-d]pyrimidin-2-yl)piperidine-4-carboxamide

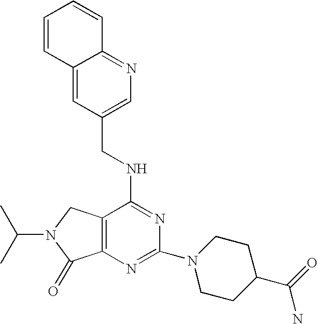

Following a procedure similar to that described in General Procedure 5 and after purification by preparative LCMS (high pH), the title compound was obtained as a solid (51.0 mg, 40.8%) following lyophilization from CH₃CN/H₂O. HPLC purity: >99% (215 nm), >99% (254 nm), >99% (280 nm); Rₜ: 0.842 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD) δ ppm 1.32 (d, J=6.64 Hz, 6H), 1.50 (qd, J=12.50, 12.11, 3.91 Hz, 2H), 1.74 (dd, J=12.89, 2.34 Hz, 2H), 2.44 (tt, J=11.91, 3.71 Hz, 1H), 2.84 (td, J=12.79, 2.15 Hz, 2H), 4.26 (s, 2H), 4.50-4.59 (m, 1H), 4.81 (d, J=13.28 Hz, 2H), 7.58-7.64 (m, 1H), 7.72-7.78 (m, 1H), 7.92 (dd, J=8.20, 1.17 Hz, 1H), 8.02 (d, J=8.59 Hz, 1H), 8.33 (d, J=1.17 Hz, 1H), 8.91 (d, J=1.95 Hz, 1H). HRMS m/z calcd for C₂₅H₃₀N₇O₂ [M+H]⁺ 460.2456, found 460.2458.

### Example 210

### 6-isopropyl-2-(4-methyl-3-oxopiperazin-1-yl)-4-(quinolin-3-ylmethylamino)-5H-pyrrolo[3,4-d]pyrimidin-7(6H)-one

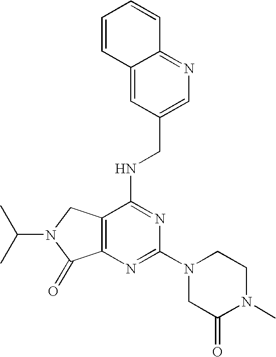

Following a procedure similar to that described in General Procedure 5 and after purification by preparative LCMS (high pH), the title compound was obtained as a solid (87.0 mg, 70.4%) following lyophilization from CH₃CN/H₂O. HPLC purity: >97% (215 nm), >96% (254 nm), >97% (280 nm); Rₜ: 0.978 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD) δ ppm 1.33 (d, J=6.64 Hz, 6H), 2.96 (s, 3H), 3.36 (t, 2H), 4.04 (t, 2H), 4.28 (s, 2H), 4.38 (s, 2H), 4.51-4.59 (m, 1H), 4.94 (s, 2H), 7.60-7.65 (m, 1H), 7.74-7.79 (m, 1H), 7.96 (d, J=8.20 Hz, 1H), 8.03 (d, J=8.59 Hz, 1H), 8.35 (d, J=1.56 Hz, 1H), 8.93 (d, J=2.34 Hz, 1H). M.S. 446.3 (ESI) (MH⁺). HRMS m/z calcd for C₂₄H₂₈N₇O₂ [M+H]⁺ 446.2299, found 446.2294.

### Example 211

### 6-isopropyl-2-morpholino-4-(quinolin-3-ylmethylamino)-5H-pyrrolo[3,4-d]pyrimidin-7(6H)-one

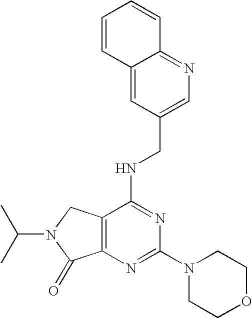

Following a procedure similar to that described in General Procedure 5 and after purification by preparative LCMS (high pH), the title compound was obtained as a solid (99.0 mg, 87.0%) following lyophilization from CH₃CN/H₂O. HPLC purity: >99% (215 nm), >99% (254 nm), >99% (280 nm); Rₜ: 0.999 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD) δ ppm 1.33 (d, J=7.03 Hz, 6H), 3.60-3.64 (m, 4H), 3.74 (t, 4H), 4.28 (s, 2H), 4.51-4.59 (m, 1H), 4.91 (s, 2H), 7.60-7.66 (m, 1H), 7.74-7.79 (m, 1H), 7.93 (dd, J=8.20, 1.17 Hz, 1H), 8.03 (d, J=8.59 Hz, 1H), 8.33 (d, J=1.17 Hz, 1H), 8.92 (d, J=2.34 Hz, 1H). M.S. 419.2 (ESI) (MH⁺). HRMS m/z calcd for C₂₃H₂₇N₆O₂ [M+H]⁺ 419.2190, found 419.2193.

### Example 212-218

In the following table were prepared following a procedure similar to that described in General Procedure 5, substituting for the appropriate starting materials.

| | | | | LCMS |
|---|---|---|---|---|
| Example | Example | | MS | Purity/retention |
| # | Name | Structure | (M + H⁺) | time |
| 212 | 6-isopropyl-2-[2-(morpholinomethyl)pyrrolidin-1-yl]-4-(3-quinolylmethylamino)-5H-pyrrolo[3,4-d]pyrimidin-7-one | 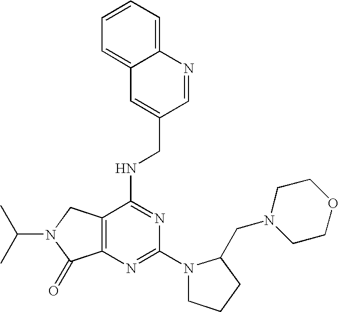 | m/z 502.2 | >99%; 1.25 min |
| 213 | 2-(3-dimethylaminopyrrolidin-1-yl)-6-isopropyl-4-(3-quinolylmethylamino)-5H-pyrrolo[3,4-d]pyrimidin-7-one | 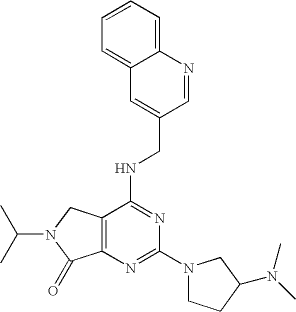 | m/z 446.3 | >97%; 0.83 min |
| 214 | 6-isopropyl-4-(3-quinolylmethylamino)-2-(quinuclidin-3-ylamino)-5H-pyrrolo[3,4-d]pyrimidin-7-one | 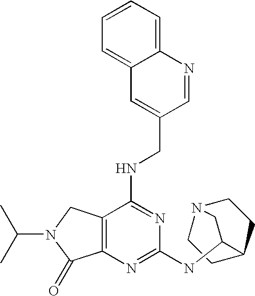 | m/z 458.3 | >96%; 0.99 min |
| 215 | 6-isopropyl-2-(2-methylsulfonylethylamino)-4-(3-quinolylmethylamino)-5H-pyrrolo[3,4-d]pyrimidin-7-one | 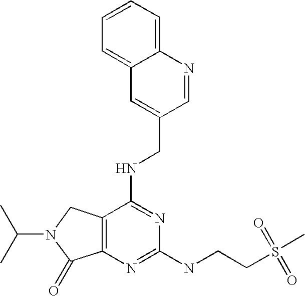 | m/z 455.3 | >97%; 0.85 min |
| 216 | 2-(3,3-difluoropyrrolidin-1-yl)-6-isopropyl-4-(3-quinolymethylamino)-5H-pyrrolo[3,4-d]pyrimidin-7-one | 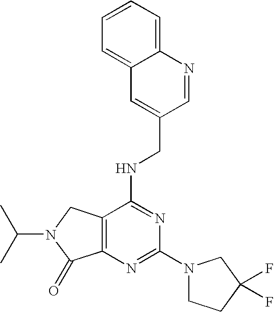 | m/z 439.3 | >99%; 1.29 min |
| 217 | 6-isopropyl-2-(methyl-(tetrahydrofuran-2-ylmethyl)amino)-4-(3-quinolylmethylamino)-5H-pyrrolo[3,4-d]pyrimidin-7-one | 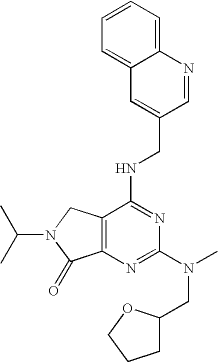 | m/z 447.3 | >99%; 1.24 min |
| 218 | 2-(4-dimethylamino-1-piperidyl)-6-isopropyl-4-(3-quinolymethylamino)-5H-pyrrolo[3,4-d[pyrimidin-7-one | 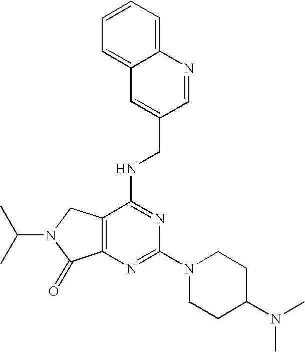 | m/z 460.2 | >99%; 0.87 min |
| 219 | 6-(1-methylethyl)-4-[(quinolin-3-ylmethyl)amino]-2-[(tetrahydro-2H-pyran-4-ylmethyl)amino]-5,6-dihydro-7H-pyrrolo[3,4-d]pyrimidin-7-one | 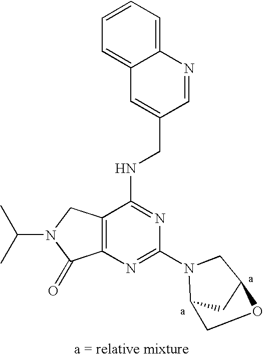 | m/z 447.3 | >99%; 1.08 min |
| 220 | 2-(dimethylamino)-6-(1-methylethyl)-4-[(quinolin-3-ylmethyl)amino]-5,6-dihydro-7H-pyrrolo[3,4-d]pyrimidin-7-one | 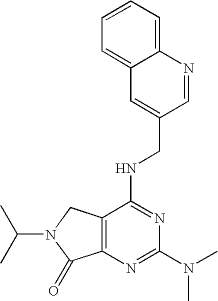 | m/z 377.2 | >96%; 1.01 min |
| 221 | N,N-dimethyl-4-{6-(1-methylethyl)-7-oxo-4-[(quinolin-3-ylmethyl)amino]-6,7-dihydro-5H-pyrrolo[3,4-d]pyrimidin-2-yl}piperazine-1-carboxamide | 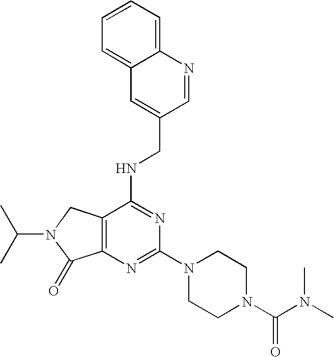 | m/z 489.2 | >95%; 1.18 min |
| 222 | 2-[4-(cyclopropylcarbonyl)piperazin-1-yl]-6-(1-methylethyl)-4-[(quinolin-3-ylmethyl)amino]-5,6-dihydro-7H-pyrrolo[3,4-d]pyrimidin-7-one | 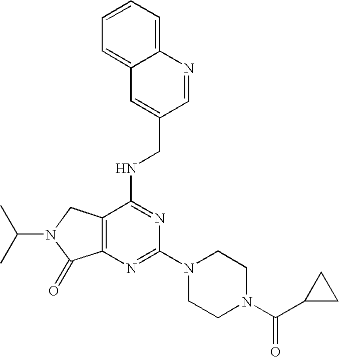 | m/z 486.2 | >99%; 1.22 min |

Note:
Column: Zorbax SB C-18;
Gradient: 05-95% B in 4.5 min, 70° C.;
Solvents: A: 0.05% TFA in H₂O, B: 0.05% TFA in MeCN, T₀ = 0.132 min

### Example 223

### 2-(4-acetylpiperazin-1-yl)-4-(2-(4-ethylpiperazin-1-yl)-4-(trifluoromethyl)benzylamino)-6-isopropyl-5H-pyrrolo[3,4-d]pyrimidin-7(6H)-one

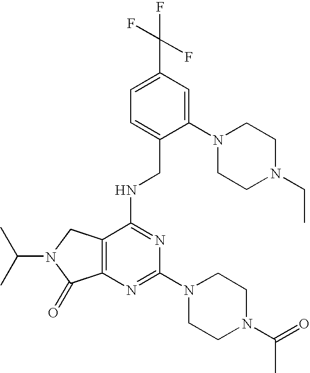

Following a procedure similar to that described in General Procedure 1 and after purification by preparative LCMS (conditions: eluting with 25-45% acetonitrile/water, pH=10), the title compound (47 mg, 26.7%) was obtained as a solid. HPLC: k′ 11.63; Purity: >93% (215 nm), >98% (254 nm), >99% (280 nm); Rₜ: 1.326 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.03 (t, J=7.1 Hz, 3H), 1.20 (d, J=6.6 Hz, 6H), 1.99 (s, 3H), 2.35-2.45 (m, 2H), 2.50-2.65 (m, 6H), 2.87-2.97 (m, 4H), 3.25-3.40 (m, 4H), 3.45-3.65 (m, 4H), 4.16 (s, 2H), 4.30-4.42 (m, 1H), 7.30 (s, 1H), 7.32 (d, J=8.0 Hz, 1H), 7.49 (d, J=8.0 Hz, 1H). M.S. (calcd): 589.7 (MH⁺), M.S. (found): 589.3 (ESI) (MH⁺).

### Example 224

### 2-(4-acetylpiperazin-1-yl)-6-isopropyl-4-(1-m-tolylpyrrolidin-3-ylamino)-5H-pyrrolo[3,4-d]pyrimidin-7(6H)-one

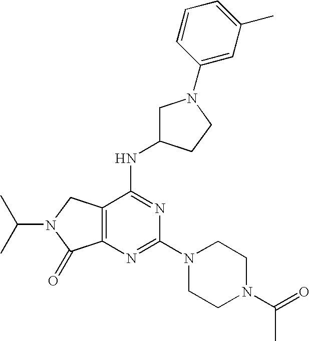

Following a procedure similar to that described in General Procedure 1 and after purification by preparative LCMS (conditions: eluting with 45-65% acetonitrile/water, pH=10), the title compound (57 mg, 40.0%) was obtained as a solid. HPLC: k′ 16.62; Purity: >93% (215 nm), >96% (254 nm), >99% (280 nm); Rₜ: 1.850 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.16 (d, J=6.7 Hz, 3H), 1.17 (d, J=7.0 Hz, 3H), 2.02 (s, 3H), 2.02-2.12 (m, 1H), 2.22 (s, 3H), 2.22-2.35 (m, 1H), 3.12-3.22 (m, 1H), 3.35-3.44 (m, 1H), 3.44-3.52 (m, 4H), 3.57-3.67 (m, 1H), 3.66-3.82 (m, 4H), 4.08 (s, 2H), 4.30-4.40 (m, 1H), 4.62-4.75 (m, 1H), 6.34 (d, J=8.0 Hz, 1H), 6.35 (s, 1H), 6.42 (d, J=7.6 Hz, 1H), 7.03 (t, J=8.0 Hz, 1H), 7.63 (d, J=6.6 Hz, 1H). M.S. (calcd): 478.6 (MH⁺), M.S. (found): 478.3 (ESI) (MH⁺).

### Example 225

### 2-(4-acetylpiperazin-1-yl)-6-isopropyl-4-(2-methoxy-4-(trifluoromethyl)benzylamino)-5H-pyrrolo[3,4-d]pyrimidin-7(6H)-one

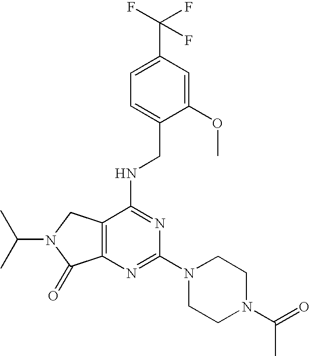

Following a procedure similar to that described in General Procedure 1 and after purification by preparative LCMS (conditions: eluting with 35-55% acetonitrile/water, pH=10), the title compound (57 mg, 37.7%) was obtained as a solid. HPLC: k′ 16.23; Purity: >99% (215 nm), >99% (254 nm), >99% (280 nm); Rₜ: 1.809 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.19 (d, J=7.8 Hz, 6H), 2.00 (s, 3H), 3.33-3.42 (m, 6H), 3.50-3.67 (m, 4H), 3.92 (s, 3H), 4.15 (s, 2H), 4.30-4.40 (m, 1H), 7.24 (d, J=7.8 Hz, 1H), 7.26 (s, 1H), 7.42 (d, J=7.8 Hz, 1H). M.S. (calcd): 507.5 (MH⁺), M.S. (found): 507.2 (ESI) (MH⁺).

### Example 226

### (R)-2-(4-acetylpiperazin-1-yl)-6-isopropyl-4-(1-(2-(trifluoromethyl)phenyl)ethylamino)-5H-pyrrolo[3,4-d]pyrimidin-7(6H)-one

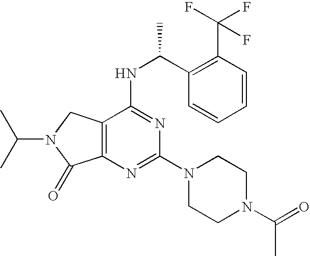

Following a procedure similar to that described in General Procedure 1 and after purification by preparative LCMS (conditions: eluting with 35-55% acetonitrile/water, pH=10), the title compound (72 mg, 49.1%) was obtained as a solid. HPLC: k′ 15.73; Purity: >97% (215 nm), >99% (254 nm), >99% (280 nm); Rₜ: 1.757 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, δ ppm 1.21 (d, J=6.7 Hz, 6H), 1.48 (d, J=7.0 Hz, 3H), 1.98 (s, 3H), 3.00-3.80 (m, 8H), 4.21 (d, J=3.1 Hz, 2H), 4.30-4.45 (m, 1H), 5.40-5.55 (m, 1H), 7.42 (dd, J=7.8, 7.4 Hz, 1H), 7.62 (dd, J=7.8, 7.4 Hz, 1H), 7.70 (d, J=7.6 Hz, 1H), 7.75 (d, J=7.6 Hz, 1H), 7.90 (d, J=6.6 Hz, 1H). M.S. (calcd): 491.5 (MH⁺), M.S. (found): 491.2 (ESI) (MH⁺).

### Example 227

### (R)-2-(4-acetylpiperazin-1-yl)-6-isopropyl-4-(1-(3-methoxyphenyl)ethylamino)-5H-pyrrolo[3,4-d]pyrimidin-7(6H)-one

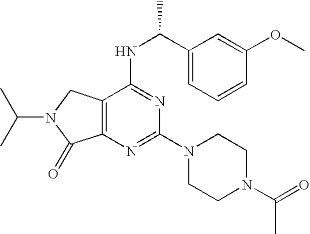

Following a procedure similar to that described in General Procedure 1 and after purification by preparative LCMS (conditions: eluting with 45-65% acetonitrile/water, pH=10), the title compound (90 mg, 53.7%) was obtained as a solid. HPLC: k′ 13.16; Purity: >99% (215 nm), >99% (254 nm), >99% (280 nm); Rₜ: 1.487 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.18 (s, 3H), 1.20 (s, 3H), 1.45 (d, J=7.03 Hz, 3H), 2.00 (s, 3H), 3.28-3.42 (m, 4H), 3.53-3.61 (m, 2H), 3.61-3.70 (m, 2H), 3.72 (s, 3H), 4.14 (s, 2H), 4.30-4.40 (m, 1H), 5.16 (s, 1H), 6.74-6.78 (m, 1H), 6.95 (s, 1H), 6.92-6.99 (m, 1H), 7.20 (t, J=7.81 Hz, 1H), 7.83 (d, J=7.81 Hz, 1H). M.S. (calcd): 453.5 (MH⁺), M.S. (found): 453.3 (ESI) (MH⁺).

### Example 228

### 2-(4-acetylpiperazin-1-yl)-6-isopropyl-4-((6-phenylpyridin-3-yl)methylamino)-5H-pyrrolo[3,4-d]pyrimidin-7(6H)-one

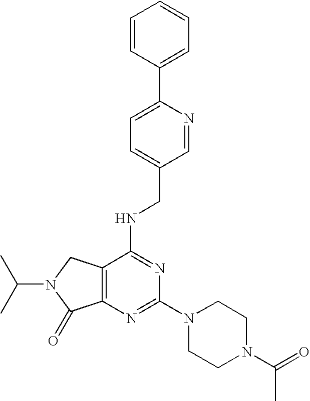

Following a procedure similar to that described in General Procedure 1 and after purification by preparative LCMS (conditions: eluting with 45-65% acetonitrile/water, pH=10), the title compound (0.106 g, 58.9%). HPLC: k′ 11.45; Purity: >97% (215 nm), >98% (254 nm), >99% (280 nm); Rₜ: 1.307 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO): δ ppm 1.18 (d, J=6.6 Hz, 6H), 1.99 (s, 3H), 3.50-3.10 (m, 4H), 3.64-3.76 (m, 4H), 4.14 (s, 2H), 4.30-4.43 (m, 1H), 4.63 (d, J=5.5 Hz, 2H), 7.37-7.44 (m, 1H), 7.44-7.51 (m, 2H), 7.83 (dd, J=8.2, 2.0 Hz, 1H), 7.91 (d, J=8.2 Hz, 1H), 8.01-8.04 (m, 1H), 8.05 (d, J=1.5 Hz, 1H), 8.09-8.17 (m, 1H), 8.67 (d, J=1.5 Hz, 1H). M.S. (calcd): 486.6 (MH⁺), M.S. (found): 486.2 (ESI) (MH⁺).

### Example 229

### 2-(4-acetylpiperazin-1-yl)-6-isopropyl-4-(isoquinolin-4-ylmethylamino)-5H-pyrrolo[3,4-d]pyrimidin-7(6H)-one

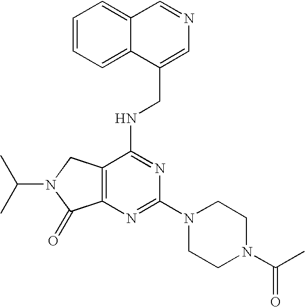

Following a procedure similar to that described in General Procedure 1 and after purification by preparative LCMS (conditions: eluting with 25-45% acetonitrile/water, pH=10), the title compound (72 mg, 52.2%) was obtained as a solid. HPLC: k′ 7.6; Purity: >97% (215 nm), >98% (254 nm), >99% (280 nm); Rₜ: 0.903 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.20 (d, J=6.7 Hz, 6H), 2.01 (s, 3H), 3.22-3.48 (m, 4H), 3.58-3.74 (m, 4H), 4.07 (s, 2H), 4.27-4.40 (m, 1H), 5.02 (s, 2H), 7.70 (dd, J=8.2, 4.3 Hz, 1H), 7.79-7.86 (m, 1H), 7.92-8.10 (m, 1H), 8.15 (d, J=8.2 Hz, 1H), 8.24 (d, J=8.2 Hz, 1H), 8.55 (s, 1H). M.S. (calcd): 460.5 (MH⁺), M.S. (found): 460.2 (ESI) (MH⁺).

### Example 230

### (R)-2-(4-acetylpiperazin-1-yl)-6-isopropyl-4-(1-(3-(trifluoromethyl)phenyl)ethylamino)-5H-pyrrolo[3,4-d]pyrimidin-7(6H)-one

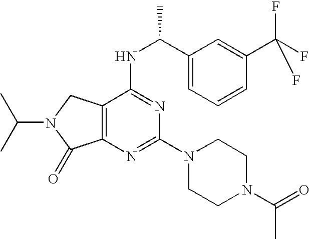

Following a procedure similar to that described in General Procedure 1 and after purification by preparative LCMS (conditions: eluting with 45-65% acetonitrile/water, pH=10), the title compound (0.118 g, 65.0%) was obtained as a solid. HPLC: k′ 16.07; Purity: >99% (215 nm), >99% (254 nm), >99% (280 nm); Rₜ: 1.792 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.19 (s, 3H), 1.21 (s, 3H), 1.49 (d, J=7.0 Hz, 3H), 1.99 (s, 3H), 3.20-3.60 (m, 8H), 3.61-3.72 (m, 1H), 4.16 (s, 2H), 4.30-4.40 (m, 1H), 5.19-5.29 (m, 1H), 7.50-7.60 (m, 2H), 7.65-7.72 (m, 1H), 7.76 (s, 1H). M.S. (calcd): 491.2 (MH⁺), M.S. (found): 491.5 (ESI) (MH⁺).

### Example 231

### 2-(4-acetylpiperazin-1-yl)-6-isopropyl-4-(4-(trifluoromethyl)benzylamino)-5H-pyrrolo[3,4-d]pyrimidin-7(6H)-one

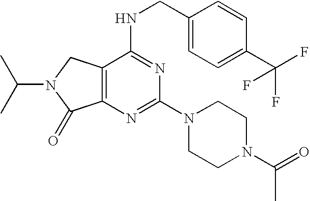

Following a procedure similar to that described in General Procedure 1 and after purification by preparative LCMS (conditions: eluting with 45-65% acetonitrile/water, pH=10), the title compound (90 mg, 54.0%) as a solid. HPLC: k′ 15.12; Purity: >99% (215 nm), >99% (254 nm), >99% (280 nm); Rₜ: 1.693 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.19 (d, J=7.0 Hz, 6H), 2.00 (s, 3H), 3.25-3.41 (m, 4H), 3.52-3.72 (m, 4H), 4.14 (s, 2H), 4.30-4.42 (m, 1H), 4.65 (d, J=5.9 Hz, 2H), 7.56 (d, J=7.8 Hz, 2H), 7.68 (d, J=7.8 Hz, 2H), 8.13 (t, J=5.9 Hz, 1H). M.S. (calcd): 477.5 (MH⁺), M.S. (found): 477.2 (ESI) (MH⁺).

### Example 232

### 2-(4-acetylpiperazin-1-yl)-6-isopropyl-4-(2-methoxy-4-(trifluoromethoxy)benzylamino)-5H-pyrrolo[3,4-d]pyrimidin-7(6H)-one

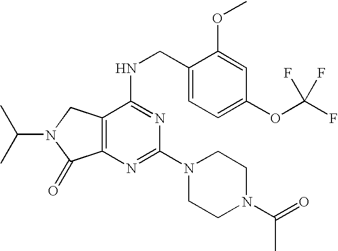

Following a procedure similar to that described in General Procedure 1 and after purification by preparative LCMS (conditions: eluting with 45-65% acetonitrile/water, pH=10), the title compound (119 mg, 61%) as a solid. HPLC: k′ 16.68; Purity: >99% (215 nm), >99% (254 nm), >99% (280 nm); Rₜ: 1.856 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.18 (d, J=6.6 Hz, 6H), 2.01 (s, 3H), 3.35-3.42 (m, 4H), 3.55-3.72 (m, 4H), 3.86 (s, 3H), 4.13 (s, 2H), 4.30-4.41 (m, 1H), 4.53 (d, J=4.3 Hz, 2H), 6.84-6.91 (m, 1H), 7.00 (d, J=2.0 Hz, 1H), 7.33 (d, J=8.2 Hz, 1H), 7.85-7.91 (m, 1H). M.S. (calcd): 523.5 (MH⁺), M.S. (found): 523.2 (ESI) (MH⁺).

### Example 233

### (R)-2-(4-acetylpiperazin-1-yl)-6-isopropyl-4-(1-(quinolin-3-yl)ethylamino)-5H-pyrrolo[3,4-d]pyrimidin-7(6H)-one AZ 12841844 and

### Example 234

### (S)-2-(4-acetylpiperazin-1-yl)-6-isopropyl-4-(1-(quinolin-3-yl)ethylamino)-5H-pyrrolo[3,4-d]pyrimidin-7(6H)-one

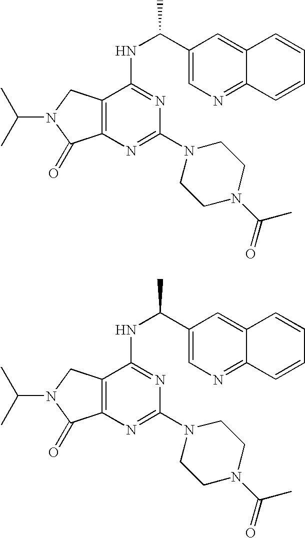

Following a procedure similar to that described in General Procedure 1 and after purification by preparative LCMS (conditions: eluting with 25-45% acetonitrile/water, pH=10), the title compound (102 mg, 59.8%) was obtained as a solid, which was further purified by chiral HPLC (CHOD column, mobile phase conditions: 7.5% methanol and 7.5% ethanol in 85% hexane with 0.1% diethyl amine, isocratic gradient, 40 minutes run) to separate the two enantiomers:

(R)-2-(4-acetylpiperazin-1-yl)-6-isopropyl-4-(1-(quinolin-3-yl)ethylamino)-5H-pyrrolo[3,4-d]pyrimidin-7(6H)-one (35.0 mg) was obtained as a solid. HPLC: k′ 9.23; Purity: >99% (215 nm), >99% (254 nm), >99% (280 nm); Rₜ: 1.074 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.17-1.25 (m, 6H), 1.62 (d, J=7.0 Hz, 3H), 1.95 (s, 3H), 3.15-3.30 (m, 4H), 3.45-3.70 (m, 4H), 4.19 (s, 2H), 4.30-4.45 (m, 1H), 5.35-5.47 (m, 1H), 7.58 (dd, J=7.8, 7.4 Hz, 1H), 7.69 (dd, J=7.8, 7.4 Hz, 1H), 7.92-8.00 (m, 2H), 8.03 (d, J=7.0 Hz, 1H), 8.28 (d, J=2.0 Hz, 1H). M.S. (calcd): 474.6 (MH⁺), M.S. (found): 474.2 (ESI) (MH⁺).

(S)-2-(4-acetylpiperazin-1-yl)-6-isopropyl-4-(1-(quinolin-3-yl)ethylamino)-5H-pyrrolo[3,4-d]pyrimidin-7(6H)-one (29.0 mg, 44.6%) was obtained as a solid. HPLC: k′ 9.20; Purity: >97% (215 nm), >98% (254 nm), >97% (280 nm); Rₜ: 1.071 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.19 (d, J=6.6 Hz, 3H), 1.20 (d, J=6.7 Hz, 3H), 1.61 (d, J=6.7 Hz, 3H), 1.95 (s, 3H), 3.15-3.33 (m, 4H), 3.45-3.70 (m, 4H), 4.19 (d, J=1.9 Hz, 2H), 4.31-4.42 (m, 1H), 5.36-5.46 (m, 1H), 7.55-7.62 (m, 1H), 7.66-7.75 (m, 1H), 7.92-8.01 (m, 1H), 8.04 (d, J=6.7 Hz, 1H), 8.28 (d, J=1.9 Hz, 1H). M.S. (calcd): 474.6 (MH⁺), M.S. (found): 474.2 (ESI) (MH⁺).

### Example 235

### 2-(4-acetylpiperazin-1-yl)-6-isopropyl-4-(quinolin-6-ylmethylamino)-5H-pyrrolo[3,4-d]pyrimidin-7(6H)-one

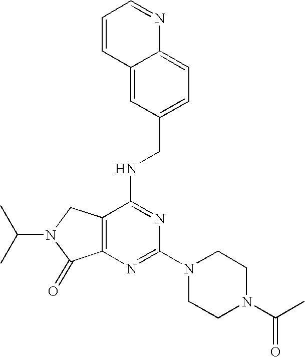

Following a procedure similar to that described in General Procedure 1 and after purification by preparative LCMS (conditions: eluting with 25-45% acetonitrile/water, pH=10), the title compound (35 mg, 25.4%) was obtained as a solid. HPLC: k′ 7.05; Purity: >99% (215 nm), >99% (254 nm), >99% (280 nm); Rₜ: 0.845 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.20 (d, J=7.0 Hz, 6H), 1.99 (s, 3H), 3.22-3.48 (m, 6H), 3.58-3.74 (m, 4H), 4.30-4.42 (m, 1H), 4.78 (d, J=5.4 Hz, 2H), 7.52 (dd, J=8.2, 4.3 Hz, 1H), 7.77 (dd, J=8.6, 1.9 Hz, 1H), 7.90-7.94 (m, 1H), 7.99 (d, J=9.0 Hz, 1H), 8.16-8.24 (m, 1H), 8.34 (d, J=7.4 Hz, 1H), 8.87 (dd, J=4.3, 1.9 Hz, 1H). M.S. (calcd): 460.5 (MH⁺), M.S. (found): 460.2 (ESI) (MH⁺). DMSO-d₆

### Example 236

### 2-(4-acetylpiperazin-1-yl)-4-(1-(benzo[d]thiazol-2-yl)ethylamino)-6-isopropyl-5H-pyrrolo[3,4-d]pyrimidin-7(6H)-one

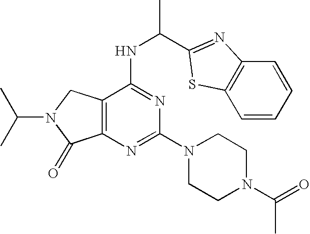

Following a procedure similar to that described in General Procedure 1 and after purification by preparative LCMS (high pH, Phenomenex Gemini C18, 21.2×250 mm, 5 μm particle size, gradient 45-65% CH₃CN in H₂O containing 10 mM NH₄HCO₃), the title compound (89 mg, 48%) was obtained as a solid. HPLC purity: >99% (215 nm), >99% (254 nm), >99% (280 nm); Rₜ: 1.53 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.29 (d, J=6.64 Hz, 6H), 1.81 (d, J=7.03 Hz, 3H), 2.09 (s, 3H), 3.30-3.41 (m, 2H), 3.52-3.59 (m, 2H), 3.72-3.84 (m, 2H), 3.85 (t, J=4.69 Hz, 2H), 4.12-4.24 (m, 2H), 4.69 (quin, J=6.74 Hz, 1H), 5.41 (d, J=6.64 Hz, 1H), 5.70 (quin, J=6.74 Hz, 1H), 7.40 (td, J=7.62, 1.17 Hz, 1H), 7.47-7.54 (m, 1H), 7.86 (d, J=7.42 Hz, 1H), 8.00 (d, J=7.81 Hz, 1H). M.S. (found): 480.2 (ESI) (MH⁺). HRMS m/z calcd for C₂₄H₃₀N₇O₂S[M+H]⁺ 480.21762, found 480.21732.

### Example 237

### 2-(4-acetylpiperazin-1-yl)-6-isopropyl-4-((1-methyl-1H-indol-2-yl)methylamino)-5H-pyrrolo[3,4-d]pyrimidin-7(6H)-one

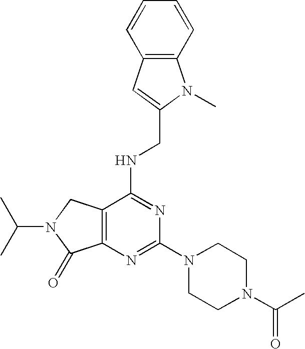

Following a procedure similar to that described in General Procedure 1 and after purification by preparative LCMS (conditions: eluting with 25-45% acetonitrile/water, pH=10), the title compound (38 mg, 27.7%) was obtained as a solid. HPLC: k′ 14.63; Purity: >95% (215 nm), >97% (254 nm), >97% (280 nm); Rₜ: 1.642 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.17 (d, J=6.6 Hz, 6H), 2.02 (s, 3H), 3.40-3.50 (m, 4H), 3.74 (s, 3H), 3.66-3.83 (m, 4H), 4.10 (s, 2H), 4.30-4.40 (m, 1H), 4.80 (d, J=4.7 Hz, 2H), 6.43 (s, 1H), 6.98 (dd, J=7.8, 7.0 Hz, 1H), 7.10 (dd, J=7.8, 7.5 Hz, 1H), 7.41 (d, J=8.0 Hz, 1H), 7.47 (d, J=8.0 Hz, 1H), 7.91 (d, J=5.8 Hz, 1H). M.S. (calcd): 462.6 (MH⁺), M.S. (found): 462.3 (ESI) (MH⁺).

### Example 238

### 2-(4-acetylpiperazin-1-yl)-6-isopropyl-4-(isoquinolin-3-ylmethylamino)-5H-pyrrolo[3,4-d]pyrimidin-7(6H)-one

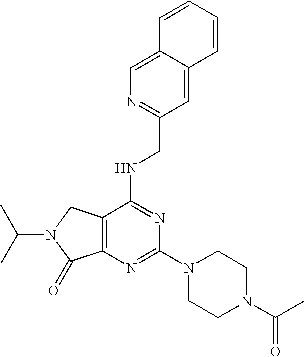

Following a procedure similar to that described in General Procedure 1 and after purification by preparative LCMS (conditions: eluting with 25-45% acetonitrile/water, pH=10), the title compound (50.7 mg, 54.1%) was obtained as a solid by preparative LCMS, eluted with 25-45% acetonitrile/water, pH=10. HPLC: k′ 8.49; Purity: >99% (215 nm), >99% (254 nm), >99% (280 nm); Rₜ: 0.996 minute; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.20 (d, J=6.6 Hz, 6H), 1.95 (s, 3H), 3.23-3.39 (m, 4H), 3.52-3.67 (m, 4H), 4.18 (s, 2H), 4.32-4.42 (m, 1H), 4.82 (d, J=5.8 Hz, 2H), 7.58-7.66 (m, 1H), 7.69-7.78 (m, 2H), 7.91 (d, J=7.8 Hz, 1H), 8.09 (d, J=8.2 Hz, 1H), 8.16-8.24 (m, 1H). M.S. (calcd): 460.5 (MH⁺), M.S. (found): 460.2 (ESI) (MH⁺).

### Example 239

### 2-(4-acetylpiperazin-1-yl)-6-isopropyl-4-(methyl(quinolin-6-ylmethyl)amino)-5H-pyrrolo[3,4-d]pyrimidin-7(6H)-one

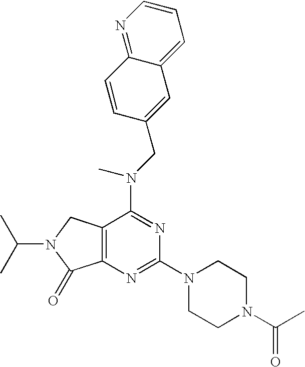

Following a procedure similar to that described in General Procedure 1 and after purification by preparative LCMS (conditions: eluting with 25-45% acetonitrile/water, pH=10), the title compound (50 mg, 25.8%) was obtained as a solid. HPLC: k′ 8.00; Purity: >99% (215 nm), >98% (254 nm), >98% (280 nm); Rₜ: 0.945 minute; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.17 (d, J=6.6 Hz, 6H), 1.97 (s, 3H), 3.25-3.55 (m, 4H), 3.27 (s, 3H), 3.55-3.75 (m, 4H), 4.25-4.45 (m, 1H), 4.58 (s, 2H), 5.02 (s, 2H), 7.50 (dd, J=8.2, 4.3 Hz, 1H), 7.68 (dd, J=9.0, 1.9 Hz, 1H), 7.83 (s, 1H), 7.99 (d, J=8.6 Hz, 1H), 8.33 (d, J=7.8 Hz, 1H), 8.85 (dd, J=4.3, 1.9 Hz, 1H). M.S. (calcd): 474.57 (MH⁺), M.S. (found): 474.2 (ESI) (MH⁺).

### Example 240

### 2-(4-acetylpiperazin-1-yl)-6-isopropyl-4-((8-methoxyquinolin-5-yl)methylamino)-5H-pyrrolo[3,4-d]pyrimidin-7(6H)-one

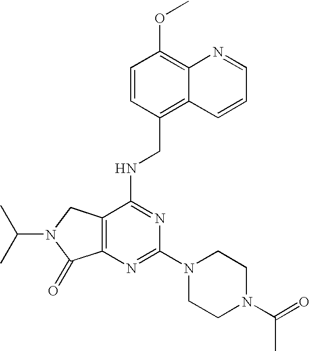

Following a procedure similar to that described in General Procedure 1 and after purification by preparative LCMS (conditions: eluting with 25-45% acetonitrile/water, pH=10), the title compound (102 mg, 69.4%) as a solid. HPLC: k′ 17.41; Purity: >99% (215 nm), >99% (254 nm), >99% (280 nm); Rₜ: 1.933 minute; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.16 (d, J=6.6 Hz, 6H), 2.03 (s, 3H), 3.30-3.50 (m, 4H), 3.65-3.80 (m, 4H), 4.03 (s, 3H), 4.11 (s, 2H), 4.25-4.40 (m, 1H), 5.03 (d, J=5.5 Hz, 2H), 7.38 (d, J=8.2 Hz, 1H), 7.74 (d, J=8.2 Hz, 1H), 7.86 (dd, J=8.6, 4.7 Hz, 1H), 8.30-8.40 (m, 1H), 8.96 (d, J=8.6 Hz, 1H), 8.98 (d, J=4.7 Hz, 1H). M.S. (calcd): 490.6 (MH⁺), M.S. (found): 490.3 (ESI) (MH⁺).

### Example 241

### 2-(4-acetylpiperazin-1-yl)-6-isopropyl-4-((2-methylquinolin-3-yl)methylamino)-5H-pyrrolo[3,4-d]pyrimidin-7(6H)-one

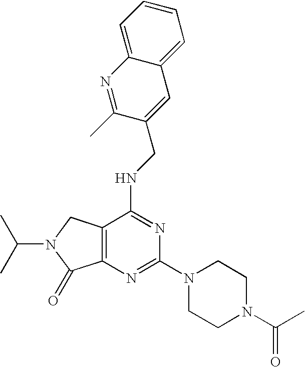

Following a procedure similar to that described in General Procedure 1 and starting from (2-methylquinolin-3-yl)methenamine (Intermediate 64), after purification by preparative LCMS (conditions: eluting with 25-45% acetonitrile/water, pH=10), the title compound (122 mg, 49.3%) was obtained as a solid. HPLC: k′ 8.14; Purity: >98% (215 nm), >99% (254 nm), >99% (280 nm); Rₜ: 0.960 minute; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.17 (d, J=6.6 Hz, 6H), 1.97 (s, 3H), 2.67 (s, 3H), 3.40-3.25 (m, 4H), 3.70-3.50 (m, 4H), 4.16 (s, 2H), 4.30-4.40 (m, 1H), 4.73 (d, J=3.7 Hz, 2H), 7.45-7.53 (m, 1H), 7.60-7.70 (m, 1H), 7.87 (dd, J=9.4, 8.2 Hz, 2H), 8.00-8.10 (m, 1H), 8.16 (s, 1H). M.S. (calcd): 474.57 (MH⁺), M.S. (found): 474.2. (ESI) (MH⁺). HRMS m/z calcd for C₂₆H₃₁N₇O₂ [M+H]⁺ 474.2539, found 474.2607.

### Example 242

### 2-(4-acetylpiperazin-1-yl)-6-isopropyl-4-((2-methylquinolin-6-yl)methylamino)-5H-pyrrolo[3,4-d]pyrimidin-7(6H)-one

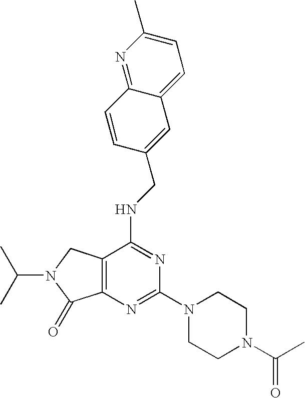

Following a procedure similar to that described in General Procedure 1 and after purification by preparative LCMS (conditions: eluting with 25-45% acetonitrile/water, pH=10), the title compound (69 mg, 35.5%) was obtained as a solid. HPLC: k′ 7.05; Purity: >99% (215 nm), >98% (254 nm), >99% (280 nm); Rₜ: 0.845 minute; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.18 (d, J=6.6 Hz, 6H), 1.97 (s, 3H), 2.62 (s, 3H), 3.25-3.40 (m, 4H), 3.55-3.75 (m, 4H), 4.14 (s, 2H), 4.30-4.40 (m, 1H), 4.74 (d, J=5.8 Hz, 2H), 7.37 (d, J=8.6 Hz, 1H), 7.69 (dd, J=8.6 Hz, 2.0 Hz, 1H), 7.84 (s, 1H), 7.86 (d, J=8.6 Hz, 1H), 8.12-8.18 (m, 1H), 8.20 (d, J=8.2 Hz, 1H). M.S. (calcd): 474.57 (MH⁺), M.S. (found): 474.49 (ESI) (MH⁺).

### Example 243

### (R)-2-(4-acetylpiperazin-1-yl)-6-isopropyl-4-(1-(quinolin-6-yl)ethylamino)-5H-pyrrolo[3,4-d]pyrimidin-7(6H)-one AZ 12892029 and

### Example 244

### (S)-2-(4-acetylpiperazin-1-yl)-6-isopropyl-4-(1-(quinolin-6-yl)ethylamino)-5H-pyrrolo[3,4-d]pyrimidin-7(6H)-one

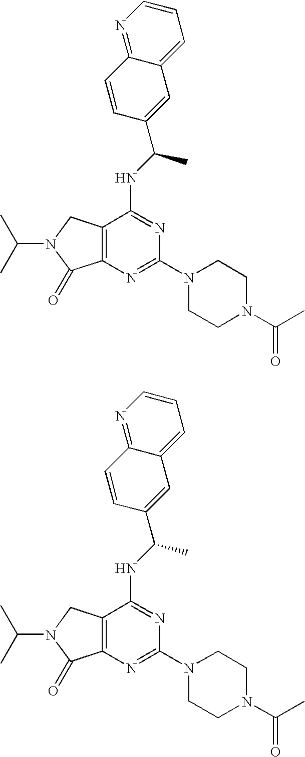

Following a procedure similar to that described in General Procedure 1 and after purification by preparative LCMS (conditions: eluting with 25-45% acetonitrile/water, pH=10), a mixture of enantiomers was obtained, which were separated by SFC using AD column, 35% isopropanol.

(R)-2-(4-acetylpiperazin-1-yl)-6-isopropyl-4-(1-(quinolin-6-yl)ethylamino)-5H-pyrrolo[3,4-d]pyrimidin-7(6H)-one (61.5 mg, 16%) was obtained as a solid. HPLC: k′ 16.37; Purity: >99% (215 nm), >99% (254 nm), >99% (280 nm); Rₜ: 1.824 minutes; Chiral SFC: conditions: AD Column with IPA+0.1% DMEA Iso at 35%; purity: >99%. Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.20 (d, J=6.6 Hz, 6H), 1.57 (d, J=7.0 Hz, 3H), 1.95 (s, 3H), 3.70-3.00 (m, 8H), 4.18 (s, 2H), 4.30-4.40 (m, 1H), 5.37 (q, J=6.6 Hz, 1H), 7.50 (dd, J=8.6, J=4.2 Hz, 1H), 7.82 (dd, J=8.8, 2.0 Hz, 1H), 7.93 (d, J=2.0 Hz, 1H), 7.97 (d, J=9.0 Hz, 1H), 8.02 (d, J=6.6 Hz, 1H), 8.34 (d, J=8.2 Hz, 1H), 8.83 (dd, J=3.9, 1.6 Hz, 1H). M.S. (calcd): 474.57 (MH⁺), M.S. (found): 474.2 (ESI) (MH⁺). HRMS m/z calcd for C₂₆H₃₁N₇O₂ [M+H]⁺ 474.2539, found 474.2612.

(S)-2-(4-acetylpiperazin-1-yl)-6-isopropyl-4-(1-(quinolin-6-yl)ethylamino)-5H-pyrrolo[3,4-d]pyrimidin-7(6H)-one (67.9 mg, 17.6%) were obtained as a solid. HPLC: k′ 16.28; Purity: >99% (215 nm), >99% (254 nm), >99% (280 nm); Rₜ: 1.814 minutes; Chiral SFC: conditions: AD Column with IPA+0.1% DMEA Iso at 35%; purity: >99%. Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.20 (d, J=6.7 Hz, 6H), 1.57 (d, J=7.0 Hz, 3H), 1.96 (s, 3H), 3.70-3.00 (m, 8H), 4.19 (s, 2H), 4.37 (q, J=7.0 Hz, 1H), 5.30-5.45 (m, 1H), 7.45-7.56 (m, 1H), 7.90-8.05 (m, 3H), 8.35 (d, J=8.6 Hz, 1H), 8.80-8.89 (m, 1H). M.S. (calcd): 474.57 (MH⁺), M.S. (found): 474.2 (ESI) (MH⁺). HRMS m/z calcd for C₂₆H₃₁N₇O₂ [M+H]⁺ 474.2539, found 474.2610.

### Example 245

### 2-(4-acetylpiperazin-1-yl)-4-(ethyl(isoquinolin-3-ylmethyl)amino)-6-isopropyl-5H-pyrrolo[3,4-d]pyrimidin-7(6H)-one

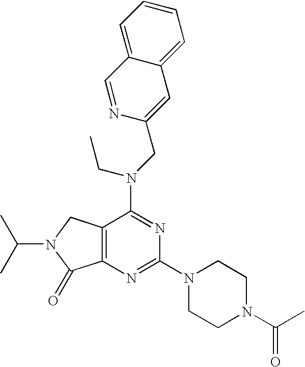

Following a procedure similar to that described in General Procedure 1, starting from N-(isoquinoline-3-ylmethyl)ethanamine (Intermediate 62B) and after purification by preparative LCMS (conditions: eluting with 25-45% acetonitrile/water, pH=10), the title compound (69 mg, 35.5%) was obtained as a solid. HPLC: k′ 12.68; Purity: >99% (215 nm), >99% (254 nm), >99% (280 nm); Rₜ: 1.436 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.05-1.30 (m, 9H), 1.97 (s, 3H), 3.20-3.40 (m, 4H), 3.50-3.65 (m, 4H), 3.68 (q, J=7.0 Hz, 2H), 4.30-4.40 (m, 1H), 4.50 (s, 2H), 4.97 (s, 2H), 7.57-7.67 (m, 1H), 7.70-7.80 (m, 2H), 7.92 (d, J=7.8 Hz, 1H), 8.10 (d, J=7.8 Hz, 1H), 9.29 (s, 1H). M.S. (calcd): 488.60 (MH⁺), M.S. (found): 488.30 (ESI) (MH⁺). HRMS m/z calcd for C₂₇H₃₃N₇O₂ [M+H]⁺ 488.2769, found 488.2767.

### Example 246

### 2-(4-acetylpiperazin-1-yl)-4-(ethyl(quinolin-3-ylmethyl)amino)-6-isopropyl-5H-pyrrolo[3,4-d]pyrimidin-7(6H)-one

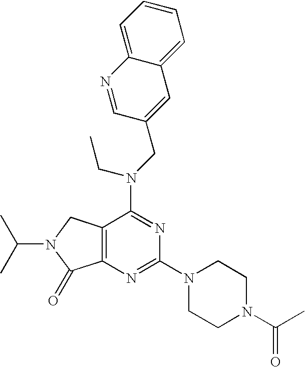

Following a procedure similar to that described in General Procedure 1, starting from N-(quinolin-3-ylmethyl)ethanamine (Intermediate 63) and after purification by preparative LCMS (conditions: eluting with 25-45% acetonitrile/water, pH=10), the title compound (140 mg, 47.1%) was obtained as a solid. HPLC: k′ 11.15; Purity: >99% (215 nm), >99% (254 nm), >99% (280 nm); Rₜ: 1.276 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.10-1.30 (m, 9H), 1.96 (s, 3H), 3.20-3.40 (m, 4H), 3.50-3.70 (m, 6H), 4.30-4.40 (m, 1H), 4.49 (s, 2H), 5.02 (s, 2H), 7.540-7.65 (m, 1H), 7.68-7.78 (m, 1H), 7.94 (d, J=8.2 Hz, 1H), 7.97-8.04 (m, 1H), 8.19 (s, 1H), 8.88 (d, J=2.3 Hz, 1H). M.S. (calcd): 488.60 (MH⁺), M.S. (found): 488.27 (ESI) (MH⁺). HRMS m/z calcd for C₂₇H₃₃N₇O₂ [M+H]⁺ 488.2766, found 488.2761.

### Example 247

### 2-(4-acetylpiperazin-1-yl)-6-isopropyl-4-((8-methylquinolin-6-yl)methylamino)-5H-pyrrolo[3,4-d]pyrimidin-7(6H)-one

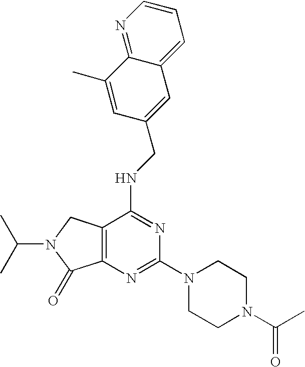

Following a procedure similar to that described in General Procedure 1 and after purification by preparative LCMS (conditions: eluting with 35-55% acetonitrile/water, pH=10), the title compound (38.0 mg, 19.75%) as a solid. HPLC: k′ 8.43; Purity: >95% (215 nm), >95% (254 nm), >97% (280 nm); Rₜ: 0.99 minute; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.18 (d, J=7.0 Hz, 6H), 1.97 (s, 3H), 2.67 (s, 3H), 3.30-3.40 (m, 4H), 3.60-3.75 (m, 4H), 4.14 (s, 2H), 4.30-4.40 (m, 1H), 4.70 (d, J=5.8 Hz, 2H), 7.49 (dd, J=8.2, 4.3 Hz, 1H), 7.62 (s, 1H), 7.72 (s, 1H), 8.10-8.19 (m, 1H), 8.28 (dd, J=8.6, 1.9 Hz, 1H), 8.84 (dd, J=4.0, 1.9 Hz, 1H). M.S. (calcd): 474.57 (MH⁺), M.S. (found): 474.2 (ESI) (MH⁺). HRMS m/z calcd for C₂₆H₃₁N₇O₂ [M+H]⁺ 474.2612, found 474.2605.

### Example 248

### 2-(4-acetylpiperazin-1-yl)-6-isopropyl-4-(quinolin-2-ylmethylamino)-5H-pyrrolo[3,4-d]pyrimidin-7(6H)-one

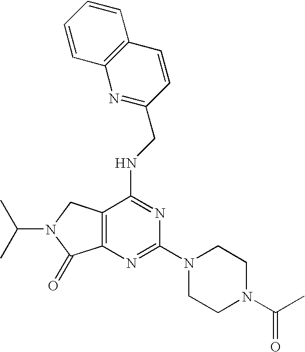

Following a procedure similar to that described in General Procedure 1 and after purification by preparative LCMS (conditions: eluting with 25-45% acetonitrile/water, pH=10), the title compound (98 mg, 26.2%) was obtained as a solid. HPLC: k′ 9.94; Purity: >93% (215 nm), >95% (254 nm), >93% (280 nm); Rₜ: 1.149 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.20 (d, J=7.1 Hz, 6H), 1.92 (s, 3H), 3.25-3.10 (m, 4H), 3.60-3.45 (m, 4H), 4.07-4.15 (m, 1H), 4.19 (s, 2H), 4.30-4.40 (m, 1H), 4.81 (d, J=5.8 Hz, 2H), 7.52 (d, J=8.6 Hz, 1H), 7.56 (m, 1H), 7.74 (m, 1H), 7.95 (dd, J=8.6 Hz, 2H), 8.29 (d, J=8.6 Hz, 1H). M.S. (calcd): 460.54 (MH⁺), M.S. (found): 460.2 (ESI) (MH⁺).

### Example 249

### 2-(4-acetylpiperazin-1-yl)-6-isopropyl-4-(methyl(quinolin-2-ylmethyl)amino)-5H-pyrrolo[3,4-d]pyrimidin-7(6H)-one

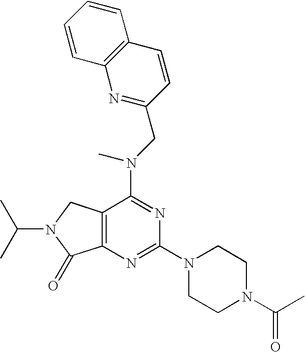

Following a procedure similar to that described in General Procedure 1, starting from N-methyl-1-(quinolin-2-yl)methanamine (Intermediate 65) and after purification by preparative LCMS (eluted with 25-45% acetonitrile/water, pH=10), the title compound (110 mg, 22.86%) was obtained as a solid. HPLC: k′ 11.86; Purity: >99% (215 nm), >99% (254 nm), >99% (280 nm); Rₜ: 1.350 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.19 (d, J=7.0 Hz, 6H), 1.93 (s, 3H), 3.30-3.10 (m, 4H), 3.41 (s, 3H), 3.55 (m, 4H), 4.32-4.42 (m, 1H), 4.63 (s, 2H), 5.04 (s, 2H), 7.42 (d, J=8.2 Hz, 1H), 7.56 (m, 1H), 7.74 (m, 1H), 7.95 (m, 2H), 8.30 (d, J=8.2 Hz, 1H). M.S. (calcd): 474.57 (MH⁺), M.S. (found): 474.2. (ESI) (MH⁺). HRMS m/z calcd for C₂₆H₃₁N₇O₂ [M+H]⁺ 474.2539, found 474.2610.

### Example 250

### (R)-2-(4-acetylpiperazin-1-yl)-4-(1-(4-(1,3-difluoropropan-2-yloxy)phenyl)ethylamino)-6-isopropyl-5H-pyrrolo[3,4-d]pyrimidin-7(6H)-one AZ 12924387 and

### Example 251

### (S)-2-(4-acetylpiperazin-1-yl)-4-(1-(4-(1,3-difluoropropan-2-yloxy)phenyl)ethylamino)-6-isopropyl-5H-pyrrolo[3,4-d]pyrimidin-7(6H)-one

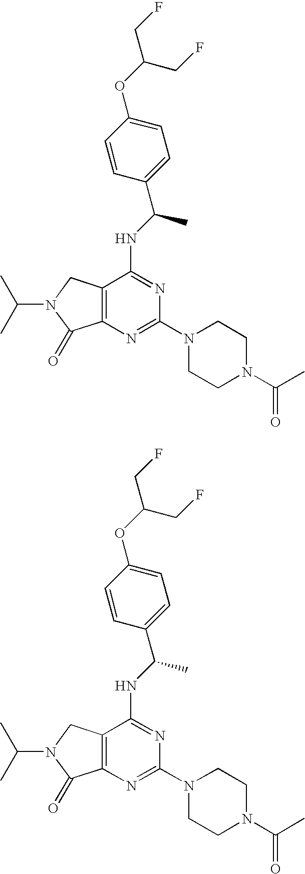

Following a procedure similar to that described in General Procedure 1 and after purification by preparative LCMS (25-45% acetonitrile/water, pH=10), a mixture of enantiomers was obtained (320 mg), which was separated by SFC.

(R)-2-(4-acetylpiperazin-1-yl)-4-(1-(4-(1,3-difluoropropan-2-yloxy)phenyl)ethylamino)-6-isopropyl-5H-pyrrolo[3,4-d]pyrimidin-7(6H)-one (41.0 mg, 9.8%) was obtained as a solid. HPLC: k′ 14.70; Purity: >98% (215 nm), >98% (254 nm), >97% (280 nm); Rₜ: 1.648 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. Chiral SFC: conditions: AD Column with IPA+1% DMEA Iso at 35%; purity: >96%. ¹H NMR (400 MHz, DMSO-d₆) δ ppm 1.15 (d, J=7.0 Hz, 6H), 1.42 (d, J=7.0 Hz, 3H), 1.97 (s, 3H), 3.40-3.30 (m, 4H), 3.70-3.50 (m, 4H), 4.09 (s, 2H), 4.25-4.40 (m, 1H), 4.53 (dd, J=9.7, 5.0 Hz, 1H), 4.55-4.90 (m, 4H), 5.05-5.20 (m, 1H), 6.92 (d, J=8.6 Hz, 2H), 7.27 (d, J=8.6 Hz, 2H). M.S. (calcd): 517.58 (MH⁺), M.S. (found): 517.3. (ESI) (MH⁺). HRMS m/z calcd for C₂₆H₃₄F₂N₆O₃ [M+H]⁺ 517.2661, found 517.2930.

(S)-2-(4-acetylpiperazin-1-yl)-4-(1-(4-(1,3-difluoropropan-2-yloxy)phenyl)ethylamino)-6-isopropyl-5H-pyrrolo[3,4-d]pyrimidin-7(6H)-one (32.0 mg, 7.6%) was obtained as a solid. HPLC: k′ 14.70; Purity: >98% (215 nm), >98% (254 nm), >97% (280 nm); Rₜ: 1.648 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. Chiral SFC: conditions: AD Column with IPA+0.1% DMEA Iso at 35%; purity: >96%. ¹H NMR (400 MHz, DMSO-d₆) δ ppm 1.15 (d, J=7.0 Hz, 6), 1.42 (d, J=7.0 Hz, 3H), 1.97 (s, 3H), 3.40-3.30 (m, 4H), 3.70-3.50 (m, 4H), 4.09 (s, 2H), 4.30-4.40 (m, 1H), 4.53 (dd, J=9.7, 5.0 Hz, 1H), 4.55-4.59 (m, 1H), 4.60-4.67 (m, 1H), 4.67-4.74 (m, 1H), 4.74-4.90 (m, 1H), 5.07-5.20 (m, 1H), 6.92 (d, J=8.6 Hz, 2H), 7.27 (d, J=8.6 Hz, 2H). M.S. (calcd): 517.58 (MH⁺), M.S. (found): 517.3. (ESI) (MH⁺). HRMS m/z calcd for C₂₆H₃₄F₂N₆O₃ [M+H]⁺ 517.2661, found 517.2933.

Note: The majority of product was not purified due to its poor solubility in alcoholic solvents. Only part of the product could be separated by SFC.

### Example 252

### 2-(4-acetylpiperazin-1-yl)-4-((1-(dimethylamino)isoquinolin-3-yl)methylamino)-6-isopropyl-5H-pyrrolo[3,4-d]pyrimidin-7(6H)-one

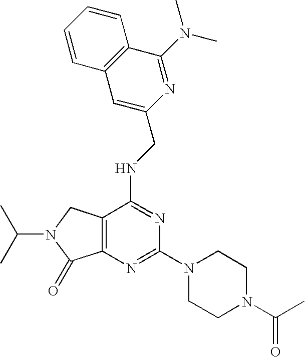

Following a procedure similar to that described in General Procedure 1, starting from 3-(aminomethyl)-N,N-dimethylisoquinolin-1-amine (Intermediate 66) and after purification by Preparative LCMS (eluted with 25-45% acetonitrile/water, pH=10), the title compound (21.0 mg, 10.3%) was obtained as a solid. HPLC: k′ 10.15; Purity: >95% (215 nm), >96% (254 nm), >95% (280 nm); Rₜ: 1.171 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. Chiral SFC: conditions: AD Column with IPA+0.1% DMEA Iso at 35%; purity: >96%. ¹H NMR (400 MHz, DMSO-d₆) δ ppm 1.19 (d, J=7.0 Hz, 6H), 1.97 (s, 3H), 3.03 (s, 6H), 3.25-3.70 (m, 8H), 3.69 (q, J=7.0 Hz, 2H), 4.18 (s, 2H), 4.30-4.45 (m, 1H), 4.65 (d, J=5.5 Hz, 2H), 7.17 (s, 1H), 7.42-7.52 (m, 1H), 7.56-7.63 (m, 1H), 7.75 (d, J=8.2 Hz, 1H), 8.04 (d, J=8.6 Hz, 1H), 8.00-8.10 (m, 1H). M.S. (calcd): 503.61 (MH⁺), M.S. (found): 503.3. (ESI) (MH⁺). HRMS m/z calcd for C₂₇H₃₄N₈O₂ [M+H]⁺ 503.2878, found 503.2873.

### Example 253

### 2-(4-acetylpiperazin-1-yl)-4-((cyclopropylmethyl)(isoquinolin-3-ylmethyl)amino)-6-isopropyl-5H-pyrrolo[3,4-d]pyrimidin-7(6H)-one

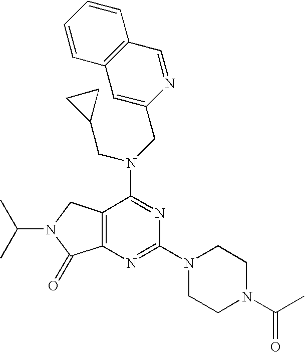

Following a procedure similar to that described in General Procedure 1, starting from 1-cyclopropyl-N-(isoquinolin-3-ylmethyl)methenamine (Intermediate 67) and after purification by Preparative LCMS (eluted with 25-45% acetonitrile/water, pH=10), the title compound (37.3 mg, 15%) was obtained as a solid. HPLC: k′ 14.66; Purity: >99% (215 nm), >99% (254 nm), >99% (280 nm); Rₜ: 1.644 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 0.23-0.35 (m, 2H), 0.38-0.50 (m, 2H), 1.02-1.28 (m, 7H), 1.97 (s, 3H), 3.2-3.80 (m, 9H), 4.25-4.39 (m, 1H), 4.40-4.56 (s, 2H), 5.05 (s, 2H), 7.64 (t, J=7.1 Hz, 1H), 7.69 (s, 1H), 7.70-7.76 (m, 1H), 7.90 (d, J=8.2 Hz, 1H), 8.08 (d, J=7.8 Hz, 1H), 9.25 (s, 1H). M.S. (calcd): 514.63 (MH⁺), M.S. (found): 514.2. (ESI) (MH⁺). HRMS m/z calcd for C₂₉H₃₅N₇O₂ [M+H] 514.2925, found 514.2931.

### Example 254

### 2-(4-acetylpiperazin-1-yl)-6-isopropyl-4-(isopropyl(isoquinolin-3-ylmethyl)amino)-5H-pyrrolo[3,4-d]pyrimidin-7(6H)-one

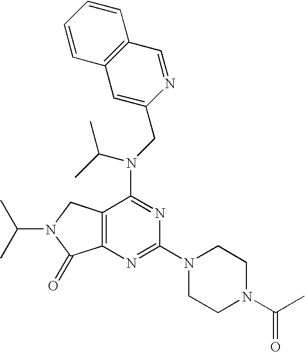

Following a procedure similar to that described in General Procedure 1, starting from N-(isoquinolin-3-ylmethyl)propan-2-amine (Intermediate 69) and after purification by preparative LCMS (eluted with 25-45% acetonitrile/water, pH=10), the title compound (74.0 mg, 30.3%) was obtained as a solid. HPLC: k′ 13.71; Purity: >95% (215 nm), >96% (254 nm), >96% (280 nm); Rₜ: 1.545 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.10-1.40 (m, 6H), 1.27 (d, J=5.4 Hz, 6H), 1.90 (s, 3H), 3.00-3.70 (m, 11H), 4.20-4.40 (m, 1H), 4.91 (s, 2H), 7.55-7.67 (m, 2H), 7.71 (t, J=7.0 Hz, 1H), 7.87 (d, J=8.6 Hz, 1H), 8.09 (d, J=8.2 Hz, 1H), 9.27 (s, 1H). M.S. (calcd): 502.62 (MH⁺), M.S. (found): 502.2. (ESI) (MH⁺). HRMS m/z calcd for C₂₈H₃₅N₇O₂ [M+H]⁺ 502.2852, found: 502.2925.

### Example 255

### 2-(4-acetylpiperazin-1-yl)-6-isopropyl-4-((isoquinolin-3-ylmethyl)(methyl)amino)-5H-pyrrolo[3,4-d]pyrimidin-7(6H)-one

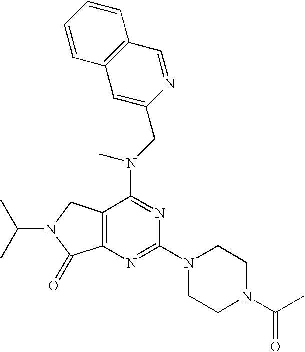

Following a procedure similar to that described in General Procedure 1, starting from 1-(isoquinolin-3-yl)-N-methylmethanamine (Intermediate 70) and after purification by Preparative LCMS (eluted with 35-55% acetonitrile/water, pH=10), the title compound (47.0 mg, 24.42%) was obtained as a solid. HPLC: k′ 11.09; Purity: >98% (215 nm), >98% (254 nm), >98% (280 nm); Rₜ: 1.269 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, DMSO-d₆) δ ppm 1.18 (d, J=6.6 Hz, 6H), 1.96 (s, 3H), 3.23-3.70 (m, 8H), 4.30-4.43 (m, 1H), 4.60 (s, 2H), 5.01 (s, 2H), 7.59-7.66 (m, 1H), 7.69 (s, 1H), 7.71-7.78 (m, 1H), 7.93 (d, J=8.2 Hz, 1H), 8.09 (d, J=8.2 Hz, 1H), 9.28 (s, 1H). M.S. (calcd): 474.57 (MH⁺), M.S. (found): 474.2 (ESI) (MH⁺). HRMS m/z calcd for C₂₆H₃₁N₇O₂ [M+H]⁺ 474.2539, found: 474.2611.

### Example 256

### 2-(4-acetylpiperazin-1-yl)-4-((2,2-difluoroethyl)(isoquinolin-3-ylmethyl)amino)-6-isopropyl-5H-pyrrolo[3,4-d]pyrimidin-7(6H)-one

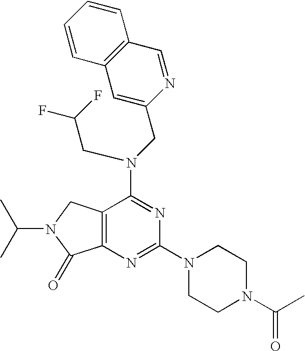

Following a procedure similar to that described in General Procedure 1, starting from 2,2-difluoro-N-(isoquinolin-3-ylmethyl)ethanamine (Intermediate 71), after purification by preparative LCMS (gradient 25-45% CH₃CN in H₂O containing 10 mM NH₄HCO₃) and lyophilization from CH₃CN/H₂O, the title compound was obtained as a solid (49.0 mg, 23.03%). HPLC purity: >99% (215 nm), >99% (254 nm), >99% (280 nm); Rₜ: 1.56 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.20 (d, J=6.6 Hz, 6H), 2.10 (s, 3H), 3.40-3.65 (m, 4H), 3.70-3.92 (m, 4H), 4.16 (dt, J=14.4, 4.0 Hz, 2H), 4.40-4.55 (m, 1H), 4.49 (s, 2H), 5.11 (s, 2H), 7.63-7.69 (m, 1H), 7.70 (s, 1H), 7.72-7.80 (m, 1H), 7.74-7.80 (m, 1H), 7.89 (d, J=7.7 Hz, 1H), 8.10 (d, J=8.2 Hz, 1H), 9.25 (s, 1H). M.S. 524.2 (ESI) (MH⁺). HRMS m/z calcd for C₂₇H₃₂FN₇O₂ [M+H]⁺ 524.2507, found 524.2580.

### Example 257

### 2-(4-acetylpiperazin-1-yl)-4-(ethyl(1-(quinolin-6-yl)ethyl)amino)-6-isopropyl-5H-pyrrolo[3,4-d]pyrimidin-7(6H)-one (Enantiomer 1)

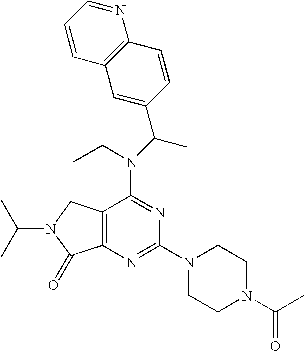

Following a procedure similar to that described in General Procedure 1, starting from 1-(quinolin-3-yl)ethanamine hydrochloride (Enantiomer 1, Intermediate 149), after purification by preparative LCMS (gradient 25-45% CH₃CN in H₂O containing 10 mM NH₄HCO₃) and lyophilization from CH₃CN/H₂O, the title compound was obtained as a solid (50.5 mg, 40.30%). HPLC purity: >98% (215 nm), >98% (254 nm), >98% (280 nm); Rₜ: 1.20 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 0.95-1.07 (m, 3H), 1.15-1.27 (m, 6H), 1.77 (d, J=6.6 Hz, 3H), 2.00 (s, 3H), 3.20-3.45 (m, 6H), 3.47-3.57 (m, 1H), 3.57-3.79 (m, 4H), 4.30-4.42 (m, 1H), 4.42-4.60 (q, J=7.2 Hz, 2H), 7.55 (dd, J=8.2, 4.3 Hz, 1H), 7.72 (dd, J=8.6, 2.0 Hz, 1H), 7.99 (s, 1H), 8.42 (d, J=8.2 Hz, 1H), 8.89 (dd, J=4.3, 1.5 Hz, 1H). M.S. 502.2. (ESI) (MH⁺). HRMS m/z calcd for C₂₈H₃₆N₇O₂ [M+H] 502.2852, found 502.2925.

### Example 258

### 2-(4-acetylpiperazin-1-yl)-4-(ethyl(1-(quinolin-6-yl)ethyl)amino)-6-isopropyl-5H-pyrrolo[3,4-d]pyrimidin-7(6H)-one (Enantiomer 2)

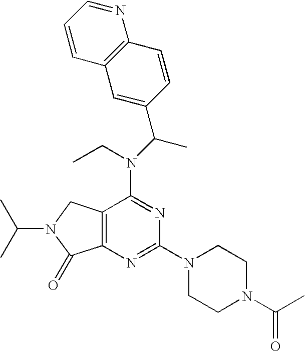

Following a procedure similar to that described in General Procedure 1, starting from 1-(quinolin-3-yl)ethanamine hydrochloride (Enantiomer 2, Intermediate 150), after purification by preparative LCMS (gradient 25-45% CH₃CN in H₂O containing 10 mM NH₄HCO₃) and lyophilization from CH₃CN/H₂O, the title compound was obtained as a solid (10.0 mg, 31.90%). HPLC purity: >99% (215 nm), >98% (254 nm), >99% (280 nm); Rₜ: 1.20 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 0.95-1.07 (m, 3H), 1.15-1.27 (m, 6H), 1.77 (d, J=6.6 Hz, 3H), 2.00 (s, 3H), 3.20-3.45 (m, 6H), 3.47-3.57 (m, 1H), 3.57-3.79 (m, 4H), 4.30-4.42 (m, 1H), 4.42-4.60 (q, J=7.2 Hz, 2H), 7.55 (dd, J=8.2, 4.3 Hz, 1H), 7.72 (dd, J=8.6, 2.0 Hz, 1H), 7.99 (s, 1H), 8.42 (d, J=8.2 Hz, 1H), 8.89 (dd, J=4.3, 1.5 Hz, 1H). M.S. 502.2. (ESI) (MH⁺). HRMS m/z calcd for C₂₈H₃₆N₇O₂ [M+H] 502.2852, found 502.2925.

### Example 259

### 2-(4-acetylpiperazin-1-yl)-6-isopropyl-4-((1-methylisoquinolin-3-yl)methylamino)-5H-pyrrolo[3,4-d]pyrimidin-7(6H)-one

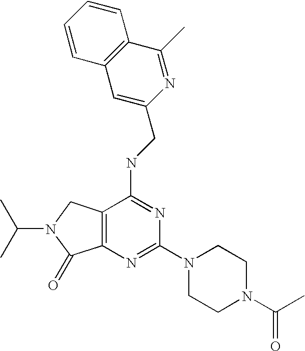

Following a procedure similar to that described in General Procedure 1, starting from (1-methylisoquinolin-3-yl)methenamine (Intermediate 72) and after purification by preparative LCMS (gradient 25-45% CH₃CN in H₂O containing 10 mM NH₄HCO₃) and lyophilization from CH₃CN/H₂O, the title compound was obtained as a solid (43.0 mg, 82.00%). HPLC purity: >99% (215 nm), >99% (254 nm), >99% (280 nm); Rₜ: 0.996 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.21 (d, J=7.0 Hz, 6H), 1.98 (s, 3H), 2.90 (s, 3H), 3.29-3.45 (m, 4H), 3.55-3.72 (m, 4H), 4.20 (s, 3H), 4.300-4.45 (m, 1H), 4.77 (s, 2H), 7.60 (s, 1H), 7.60-7.65 (m, 1H), 7.68-7.75 (m, 1H), 7.90 (d, J=8.2 Hz, 1H), 8.19 (d, J=7.4 Hz, 1H). M.S. 474.2. (ESI) (MH⁺). HRMS m/z calcd for C₂₆H₃₂N₇O₂ [M+H]⁺ 474.2539, found 474.2612.

### Example 260

### 2-(4-acetylpiperazin-1-yl)-6-isopropyl-4-(methyl(1-(quinolin-6-yl)ethyl)amino)-5H-pyrrolo[3,4-d]pyrimidin-7(6H)-one (Enantiomer 1) AZ12978200 and

### Example 261

### 2-(4-acetylpiperazin-1-yl)-6-isopropyl-4-(methyl(1-(quinolin-6-yl)ethyl)amino)-5H-pyrrolo[3,4-d]pyrimidin-7(6H)-one (Enantiomer 2)

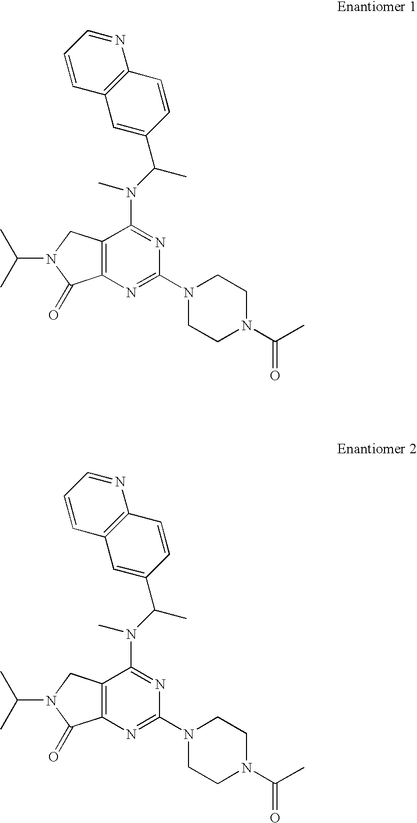

Following a procedure similar to that described in General Procedure I and after purification by preparative LCMS (gradient 25-45% CH₃CN in H₂O containing 10 mM NH₄HCO₃) and lyophilization from CH₃CN/H₂O, a mixture of two enantiomers was obtained as a solid (185 mg, 70.6%), which was further separated by SFC(OD column, with MeOH+0.1% DMEA ISO at 55° C.).

Enantiomer 1: Yielded 29.50 mg (11.27%). HPLC purity: >95% (215 nm), >95% (254 nm), >95% (280 nm); Rₜ: 1.04 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (dd, J=7.0, 3.5 Hz, 6H), 1.67 (d, J=7.0 Hz, 3H), 2.02 (s, 3H), 2.94 (s, 3H), 3.31 (s, 1H), 3.38-3.53 (m, 4H), 3.60-3.82 (m, 4H), 4.30-4.45 (m, 1H), 4.60 (d, J=7.4 Hz, 2H), 7.54 (dd, J=8.0, 4.3 Hz, 1H), 7.69 (dd, J=8.6, 1.8 Hz, 1H), 7.96 (s, 1H), 7.99 (d, J=8.6 Hz, 1H), 8.39 (d, J=8.0 Hz, 1H), 8.88 (dd, J=4.3, 1.8 Hz, 1H). M.S. 488.3. (ESI) (MH⁺). HRMS m/z calcd for C₂₇H₃₃N₇O₂ [M+H]⁺ 488.2696, found 488.2783.

Enantiomer 2: Yielded 88.00 mg (33.60%). HPLC purity: >99% (215 nm), >99% (254 nm), >99% (280 nm); Rₜ: 1.04 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (dd, J=7.0, 3.5 Hz, 6H), 1.67 (d, J=7.0 Hz, 3H), 2.02 (s, 3H), 2.94 (s, 3H), 3.31 (s, 1H), 3.38-3.53 (m, 4H), 3.60-3.82 (m, 4H), 4.30-4.45 (m, 1H), 4.60 (d, J=7.4 Hz, 2H), 7.54 (dd, J=8.0, 4.3 Hz, 1H), 7.69 (dd, J=8.6, 1.8 Hz, 1H), 7.96 (s, 1H), 7.99 (d, J=8.6 Hz, 1H), 8.39 (d, J=8.0 Hz, 1H), 8.88 (dd, J=4.3, 1.8 Hz, 1H). M.S. 488.3. (ESI) (MH⁺). HRMS m/z calcd for C₂₇H₃₃N₇O₂ [M+H]⁺ 488.2696, found 488.2770.

### Example 262

### (R)-2-(4-acetylpiperazin-1-yl)-4-((1-(4-ethoxy-3-fluorophenyl)ethyl)(methyl)amino)-6-isopropyl-5H-pyrrolo[3,4-d]pyrimidin-7(6H)-one

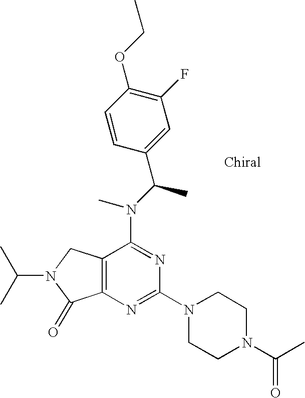

Following a procedure similar to that described in General Procedure 1, starting from Intermediate 73 and after purification by preparative LCMS (gradient 35-55% CH₃CN in H₂O containing 10 mM NH₄HCO₃) and lyophilization from CH₃CN/H₂O, the title compound was obtained as a solid (68.0 mg, 47.9%). HPLC purity: >98% (215 nm), >97% (254 nm), >98% (280 nm); Rₜ: 1.83 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (dd, J=7.0, 1.6 Hz, 6H), 1.51 (d, J=7.0 Hz, 3H), 2.03 (s, 3H), 2.88 (s, 3H), 3.31 (s, 3H), 3.34 (s, 1H), 3.40-4.53 (m, 4H), 3.60-3.80 (m, 4H), 4.08 (ABq, J=7.0 Hz, 2H), 4.30-4.45 (m, 1H), 4.57 (ABq, J=27.7 Hz, 2H), 7.00-7.25 (m, 3H). M.S. 499.3. (ESI) (MH⁺). HRMS m/z calcd for C₂₆H₃₆FN₆O₃ [M+H]⁺ 499.2755, found 499.2827.

### Example 263

### 2-(4-acetylpiperazin-1-yl)-6-isopropyl-4-(methyl((1-methylisoquinolin-3-yl)methyl)amino)-5H-pyrrolo[3,4-d]pyrimidin-7(6H)-one

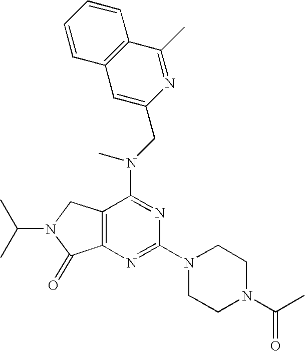

Following a procedure similar to that described in General Procedure 1, starting from N-methyl-1-(1-methylisoquinolin-3-yl)methenamine (Intermediate 74) and after purification by preparative LCMS (gradient 35-55% CH₃CN in H₂O containing 10 mM NH₄HCO₃) and lyophilization from CH₃CN/H₂O, the title compound was obtained as a solid (100.0 mg, 62.80%). HPLC purity: >96% (215 nm), >98% (254 nm), >98% (280 nm); Rₜ: 1.15 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.6 Hz, 6H), 1.97 (s, 3H), 2.87 (s, 3H), 3.34 (s, 3H), 3.32-3.44 (m, 4H), 3.55-3.72 (m, 4H), 4.32-4.45 (m, 1H), 4.64 (s, 2H), 4.96 (s, 2H), 7.52 (s, 1H), 7.63 (m, 1H), 7.69-7.77 (m, 1H), 7.90 (d, J=8.2 Hz, 1H), 8.19 (d, J=8.2 Hz, 1H). M.S. 488.2. (ESI) (MH⁺). HRMS m/z calcd for C₂₇H₃₄N₇O₂ [M+H]⁺ 488.2696, found 488.2763.

### Example 264

### 2-(4-acetylpiperazin-1-yl)-6-isopropyl-4-((isoquinolin-3-ylmethyl)(2,2,2-trifluoroethyl)amino)-5H-pyrrolo[3,4-d]pyrimidin-7(6H)-one

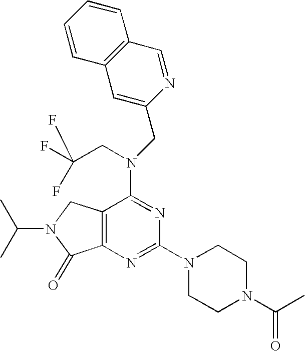

Following a procedure similar to that described in General Procedure 1, starting from 2,2,2-trifluoro-N-(isoquinolin-3-ylmethyl)ethanamine (Intermediate 75) and after purification by preparative LCMS (gradient 25-45% CH₃CN in H₂O containing 10 mM NH₄HCO₃) and lyophilization from CH₃CN/H₂O, the title compound was obtained as a solid (3.2 mg, 7.10%). HPLC purity: >99% (215 nm), >99% (254 nm), >99% (280 nm); Rₜ: 1.56 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 0.51 (d, J=7.0 Hz, 6H), 1.10-1.40 (m, 2H), 3.74 (s, 3H), 4.20-3.60 (m, 9H), 4.05 (s, 2H), 4.39 (s, 2H), 6.72-6.86 (m, 2H), 6.86-6.94 (m, 1H), 6.95-7.01 (m, 1H), 7.22-7.30 (d, J=7.8 Hz, 1H), 8.38 (s, 1H). M.S. 542.2. (ESI) (MH⁺). HRMS m/z calcd for C₂₇H₃₀F₃N₇O₂ [M+H]⁺ 542.2413, found 542.2486.

### Example 265

### benzyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate

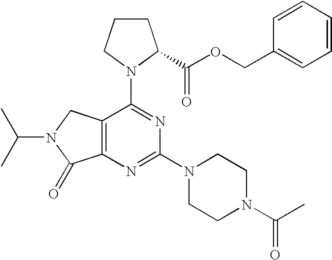

Following a procedure similar to that described in General Procedure 6 and after purification by preparative LCMS (high pH), the title compound was obtained as a solid (86 mg, 44%). HPLC: 99% (215 nm), >99% (254 nm), >99% (280 nm); Rₜ: 1.710 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CDCl₃) [symbol] ppm 1.28 (t, J=6.25 Hz, 6H), 1.43-1.84 (m, 2H), 2.08 (s, 3H), 2.14-2.39 (m, 2H), 3.23-3.41 (m, 2H), 3.52 (d, J=3.52 Hz, 2H), 3.59-3.82 (m, 5H), 3.87-4.00 (m, 1H), 4.32-4.54 (m, 2H), 4.60-4.79 (m, 2H), 5.15 (q, J=12.50 Hz, 2H), 7.12-7.41. MS [M+H]⁺ 507.2 (ESI).

### Example 266

### 4-(4-acetylpiperazin-1-yl)-2-(2-benzyl-1-piperidyl)-8-propan-2-yl-3,5,8-triazabicyclo[4.3.0]nona-1,3,5-trien-7-one

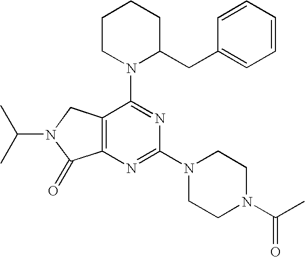

Following a procedure similar to that described in General Procedure 6 and after purification by preparative LCMS (high pH), the title compound was obtained as a solid (84 mg, 43%). HPLC purity>99% (215 nm), >99% (254 nm), >99% (280 nm); Rₜ: 1.875 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CDCl₃) δ ppm 1.23 (dd, J=11.52, 6.84 Hz, 6H), 1.54-1.68 (m, 2H), 1.69-1.80 (m, 4H), 1.81-1.94 (m, 2H), 2.15 (s, 3H), 2.86-2.97 (m, 1H), 3.00-3.11 (m, 1H), 3.29 (t, J=12.50 Hz, 1H), 3.47-3.55 (m, 2H), 3.80-3.86 (m, 2H), 3.89-4.01 (m, 3H), 4.10-4.33 (m, 2H), 4.53-4.82 (m, 2H), 7.06-7.35 (m, 5H). MS [M+H]⁺ 477.2 (ESI)

### Example 267

### 4-(4-acetylpiperazin-1-yl)-2-[2-(4-dimethylaminophenyl)pyrrolidin-1-yl]-8-propan-2-yl-3,5,8-triazabicyclo[4.3.0]nona-1,3,5-trien-7-one

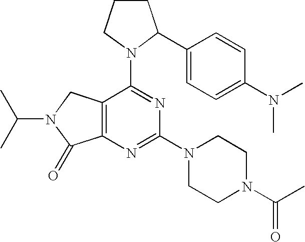

Following a procedure similar to that described in General Procedure 6 and after purification by preparative LCMS (high pH), the title compound was obtained as a solid (85 mg, 42%). HPLC purity>99% (215 nm), >99% (254 nm), >99% (280 nm); Rₜ: 1.162 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.13-1.39 (m, 6H), 1.88-2.06 (m, 3H), 2.11 (s, 3H), 2.31-2.46 (m, 1H), 2.88 (s, 6H), 3.37-3.95 (m, 10H), 3.99-4.16 (m, 1H), 4.25-4.70 (m, 2H), 5.23 (d, J=7.03 Hz, 1H), 6.75 (d, J=8.20 Hz, 2H), 7.03 (d, J=8.20 Hz, 2H). MS [M+H]⁺ 492.2 (ESI); HRMS m/z calcd for C₂₇H₃₈N₇O₂ [M+H]⁺ 492.30815, found: 492.30739.

### Example 268

### 4-(4-acetylpiperazin-1-yl)-2-[2-[(4-fluorophenyl)methyl]-1-piperidyl]-8-propan-2-yl-3,5,8-triazabicyclo[4.3.0]nona-1,3,5-trien-7-one

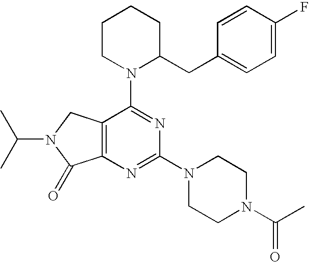

Following a procedure similar to that described in General Procedure 6 and after purification by preparative LCMS (high pH), the title compound was obtained as a solid (78 mg, 38%). HPLC purity>99% (215 nm), >99% (254 nm), >99% (280 nm); Rₜ: 1.880 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.28 (dd, J=9.57, 6.84 Hz, 6H), 1.29-1.36 (m, 2H), 1.51-1.65 (m, 1H), 1.67-1.83 (m, 4H), 1.83-1.97 (m, 1H), 2.15 (s, 3H), 2.88 (dd, J=13.67, 6.25 Hz, 1H), 3.11-3.26 (m, 1H), 3.35-3.50 (m, 1H), 3.54-3.60 (m, 2H), 3.60-3.65 (m, 2H), 3.73-3.80 (m, 2H), 3.82-3.89 (m, 2H), 3.97-4.25 (m, 1H), 4.29-4.41 (m, 1H), 4.43-4.54 (m, 1H), 6.90 (t, J=8.79 Hz, 2H), 7.20 (dd, J=8.59, 5.47 Hz, 2H). MS [M+H] 495.2 (ESI); HRMS m/z calcd for C₂₇H₃₆FN₆O₂ [M+H]⁺ 495.28783, found: 495.28670.

### Example 269

### 4-(4-acetylpiperazin-1-yl)-2-[2-(3-methylphenyl)pyrrolidin-1-yl]-8-propan-2-yl-3,5,8-triazabicyclo[4.3.0]nona-2,4,10-trien-7-one

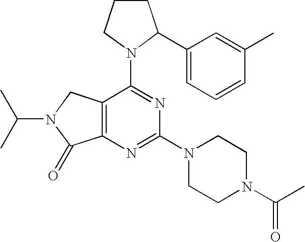

Following a procedure similar to that described in General Procedure 6 and after purification by preparative LCMS (high pH), the title compound was obtained as a solid (88 mg, 46%). HPLC purity>99% (215 nm), >99% (254 nm), >99% (280 nm); Rₜ: 1.825 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.28 (s, 6H), 1.81-2.05 (m, 3H), 2.09 (s, 3H), 2.30 (s, 3H), 2.38-2.55 (m, 1H), 3.31-4.01 (m, 10H), 4.03-4.16 (m, 1H), 4.34-4.78 (m, 2H), 5.26 (d, J=6.25 Hz, 1H), 6.97 (d, J=7.42 Hz, 1H), 7.00-7.08 (m, 2H), 7.12-7.28 (m, 1H). MS [M+H]⁺ 463.2 (ESI); HRMS m/z calcd for C₂₆H₃₅N₆O₂ [M+H]⁺ 463.28190, found: 463.28202.

### Example 270

### 4-(4-acetylpiperazin-1-yl)-2-[2-(3,5-dimethylphenyl)pyrrolidin-1-yl]-8-propan-2-yl-3,5,8-triazabicyclo[4.3.0]nona-2,4,10-trien-7-one

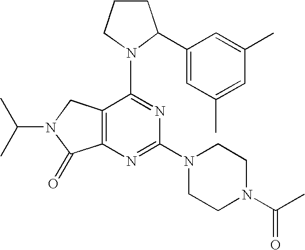

Following a procedure similar to that described in General Procedure 6 and after purification by preparative LCMS (high pH), the title compound was obtained as a solid (102 mg, 65%). HPLC purity>99% (215 nm), >97% (254 nm), >99% (280 nm); Rₜ: 1.954 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 0.84-1.62 (m, 6H), 1.75-2.05 (m, 3H), 2.08 (s, 3H), 2.24 (s, 6H), 2.32-2.50 (m, 1H), 3.40-3.74 (m, 6H), 3.80-3.86 (m, 4H), 3.95-4.17 (m, 1H), 4.31-4.78 (m, 2H), 5.20 (d, J=7.03 Hz, 1H), 6.79 (s, 2H), 6.84 (s, 1H). MS [M+H]⁺ 477.2 (ESI); HRMS m/z calcd for C₂₇H₃₇N₆O₂ [M+H]⁺ 477.29725, found: 477.29700.

### Example 271

### 4-(4-acetylpiperazin-1-yl)-2-(2-phenethylpyrrolidin-1-yl)-8-propan-2-yl-3,5,8-triazabicyclo[4.3.0]nona-1,3,5-trien-7-one

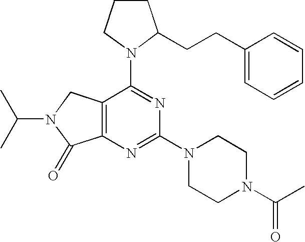

Following a procedure similar to that described in General Procedure 6 and after purification by preparative LCMS (high pH), the title compound was obtained as a solid (108 mg, 69%). HPLC purity>99% (215 nm), >99% (254 nm), >99% (280 nm); Rₜ: 1.857 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.27 (d, J=6.64 Hz, 6H), 1.61-1.77 (m, 1H), 1.93-2.06 (m, 3H), 2.11 (s, 3H), 2.11-2.23 (m, 2H), 2.56-2.76 (m, 2H), 3.47 (d, J=4.69 Hz, 2H), 3.49-3.57 (m, 2H), 3.65 (d, J=6.25 Hz, 3H), 3.74 (d, J=4.69 Hz, 3H), 4.29-4.41 (m, 1H), 4.42-4.64 (m, 3H), 7.09-7.19 (m, 3H), 7.24 (t, J=7.42 Hz, 2H). MS [M+H]⁺ 477.2 (ESI); HRMS m/z calcd for C₂₇H₃₇N₆O₂ [M+H]⁺ 477.29725, found: 477.29748.

### Example 272

### 4-(4-acetylpiperazin-1-yl)-2-[2-(4-ethoxyphenyl)pyrrolidin-1-yl]-8-propan-2-yl-3,5,8-triazabicyclo[4.3.0]nona-2,4,10-trien-7-one

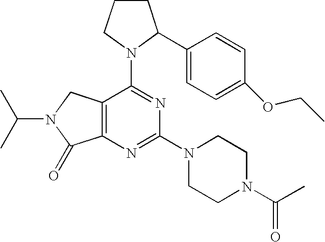

Following a procedure similar to that described in General Procedure 6 and after purification by preparative LCMS (high pH), the title compound was obtained as a solid (32 mg, 20%). HPLC purity>99% (215 nm), >99% (254 nm), >99% (280 nm); Rₜ: 1.837 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.07-1.23 (m, 6H), 1.25 (t, J=7.03 Hz, 3H), 1.72-1.98 (m, 3H), 2.01 (s, 3H), 2.23-2.38 (m, 1H), 3.32-3.84 (m, 7H), 3.90 (q, J=7.03 Hz, 2H), 3.95-4.06 (m, 1H), 4.13-4.71 (m, 5H), 5.18 (d, J=7.42 Hz, 1H), 6.74-6.84 (m, 2H), 7.01 (d, J=8.20 Hz, 2H). MS [M+H]⁺ 493.2 (ESI); HRMS m/z calcd for C₂₇H₃₇N₆O₃ [M+H]⁺ 493.29217, found: 493.29228.

### Example 273

### 4-(4-acetylpiperazin-1-yl)-2-(2-benzylazetidin-1-yl)-8-propan-2-yl-3,5,8-triazabicyclo[4.3.0]nona-1,3,5-trien-7-one

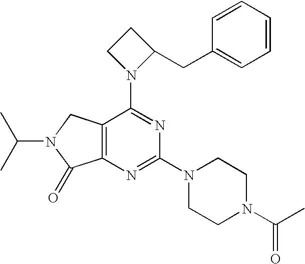

Following a procedure similar to that described in General Procedure 6 and after purification by preparative LCMS (high pH), the title compound was obtained as a solid (26 mg, 21%). HPLC purity>95% (215 nm), >99% (254 nm), >99% (280 nm); Rₜ: 1.710 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.27 (d, J=6.64 Hz, 6H), 2.12 (s, 3H), 2.15-2.25 (m, 1H), 2.32-2.44 (m, 1H), 3.11 (dd, J=13.28, 8.20 Hz, 1H), 3.35 (dd, J=13.48, 3.71 Hz, 1H), 3.51-3.65 (m, 4H), 3.79 (q, J=4.56 Hz, 2 H), 3.83-3.90 (m, 2H), 3.97-4.08 (m, 1H), 4.15 (q, J=7.55 Hz, 1H), 4.24-4.40 (m, 2H), 4.43-4.55 (m, 1H), 4.71-4.81 (m, 1H), 7.16-7.23 (m, 3H), 7.24-7.31 (m, 2H). MS [M+H]⁺ 449.2 (ESI); HRMS m/z calcd for C₂₅H₃₃N₆O₂ [M+H]⁺ 449.26695, found: 449.26675.

### Example 274

### 4-(4-acetylpiperazin-1-yl)-8-propan-2-yl-2-(2-quinolin-3-ylpyrrolidin-1-yl)-3,5,8-triazabicyclo[4.3.0]nona-1,3,5-trien-7-one

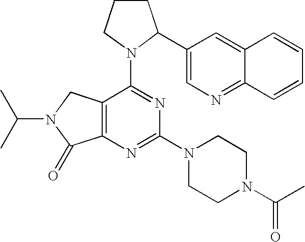

Following a procedure similar to that described in General Procedure 6 and after purification by preparative LCMS (high pH), the title compound was obtained as a solid (38 mg, 23%). HPLC purity>99% (215 nm), >99% (254 nm), >99% (280 nm); Rₜ: 1.221 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.25 (d, J=2.73 Hz, 6H), 1.85 (s, 3H), 1.90-2.01 (m, 1H), 2.04-2.21 (m, 2H), 2.42-2.55 (m, 1H), 2.72-3.15 (m, 4H), 3.26-3.69 (m, 4H), 3.92 (d, J=7.81 Hz, 1H), 4.10-4.21 (m, 1H), 4.38-4.51 (m, 1H), 4.58-4.72 (m, 2H), 5.43 (d, J=3.52 Hz, 1H), 7.50 (t, J=7.42 Hz, 1H), 7.64 (t, J=7.42 Hz, 1H), 7.80 (d, J=8.20 Hz, 1H), 7.92 (d, J=8.20 Hz, 1H), 8.08 (s, 1H), 8.73 (s, 1H). MS [M+H]⁺ 500.3 (ESI); HRMS m/z calcd for C₂₈H₃₄N₇O₂ [M+H]⁺ 500.27685, found: 500.27660.

### Example 275

### 4-(4-acetylpiperazin-1-yl)-2-[2-(1-phenylpropyl)pyrrolidin-1-yl]-8-propan-2-yl-3,5,8-triazabicyclo[4.3.0]nona-2,4,10-trien-7-one

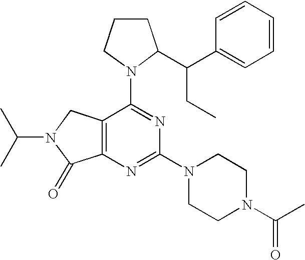

Following a procedure similar to that described in General Procedure 6 and after purification by preparative LCMS (high pH), the title compound was obtained as a solid (66 mg, 41%). HPLC purity>99% (215 nm), >99% (254 nm), >99% (280 nm); Rₜ: 1.963 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 0.72 (t, J=7.23 Hz, 3H), 1.29 (d, J=6.64 Hz, 6H), 1.62-1.95 (m, 4H), 1.95-2.10 (m, 2H), 2.14 (s, 3H), 3.18-3.26 (m, 1H), 3.55-3.61 (m, 2H), 3.64 (t, J=4.88 Hz, 2H), 3.72 (t, J=6.84 Hz, 2H), 3.79-3.97 (m, 4H), 4.33-4.70 (m, 4H), 6.99-7.35 (m, 5H). MS [M+H]⁺ 491.2 (ESI); HRMS m/z calcd for C₂₈H₃₉N₆O₂ [M+H]⁺ 491.31290, found: 491.31270.

### Example 276

### 4-(4-acetylpiperazin-1-yl)-2-[(3-cyclopentyl-1,2,4-oxadiazol-5-yl)methylamino]-8-propan-2-yl-3,5,8-triazabicyclo[4.3.0]nona-1,3,5-trien-7-one

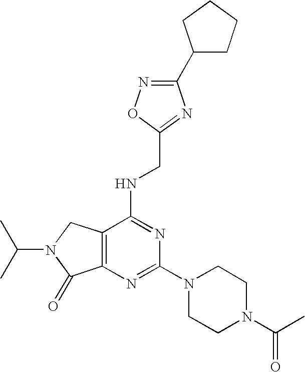

Following a procedure similar to that described in General Procedure 6 and after purification by preparative LCMS (high pH), the title compound was obtained as a solid (108 mg, 70%). HPLC purity>99% (215 nm), >99% (254 nm), >99% (280 nm); Rₜ: 1.478 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.11 (dd, J=8.98, 6.25 Hz, 2H), 1.28 (d, J=7.03 Hz, 6H), 1.61-1.90 (m, 4H), 2.10 (s, 3H), 2.07-2.13 (m, 2H), 3.31-3.40 (m, 1H), 3.45-3.58 (m, 4H), 3.69-3.76 (m, 2H), 3.78-3.84 (m, 2H), 4.21 (s, 2H), 4.43-4.58 (m, 1H), 4.69 (s, 2H). MS [M+H]⁺ 469.2 (ESI); HRMS m/z calcd for C₂₃H₃₃N₈O₃ [M+H]⁺ 469.26701, found: 469.26720.

### Example 277

### tert-butyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate

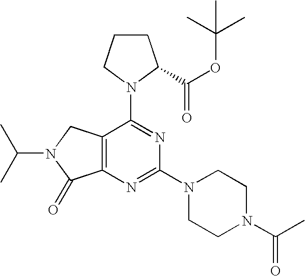

Following a procedure similar to that described in General Procedure 6 and after purification by preparative LCMS (high pH), the title compound was obtained as a solid (38 mg, 24%). HPLC purity>99% (215 nm), >99% (254 nm), >99% (280 nm); Rₜ: 1.645 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.30 (dd, J=6.25, 3.52 Hz, 6H), 1.40 (s, 9H), 1.96-2.15 (m, 3H), 2.11 (s, 3H), 2.19-2.35 (m, 1H), 3.50-3.61 (m, 4H), 3.76 (d, J=5.08 Hz, 2H), 3.79-3.93 (m, 4H), 4.46-4.57 (m, 2H), 4.58-4.68 (m, 2H). MS [M+H]⁺ 473.2 (ESI); HRMS m/z calcd for C₂₄H₃₇N₆O₄ [M+H]⁺ 473.28708, found: 473.28697.

### Example 278

### 4-(4-acetylpiperazin-1-yl)-2-[(5-cyclobutyl-1,2,4-oxadiazol-3-yl)methylamino]-8-propan-2-yl-3,5,8-triazabicyclo[4.3.0]nona-1,3,5-trien-7-one

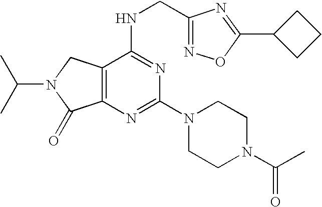

Following a procedure similar to that described in General Procedure 6 and after purification by preparative LCMS (high pH), the title compound was obtained as a solid (105 mg, 70%). HPLC purity>97% (215 nm), >97% (254 nm), >97% (280 nm); Rₜ: 1.326 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.18 (d, J=5.86 Hz, 1H), 1.29 (d, J=6.64 Hz, 6H), 1.95-2.03 (m, 1H), 2.10 (s, 3H), 2.12-2.19 (m, 1H), 2.32-2.49 (m, 3H), 3.44-3.60 (m, 4H), 3.68-3.77 (m, 2H), 3.76-3.80 (m, 1H), 3.78-3.85 (m, 2H), 4.22 (s, 2H), 4.44-4.57 (m, 1H), 4.71 (s, 2H). MS [M+H]⁺ 455.3 (ESI); HRMS m/z calcd for C₂₂H₃₁N₈O₃ [M+H]⁺ 455.20136, found: 455.26107.

### Example 279

### 4-(4-acetylpiperazin-1-yl)-2-[(2R)-2-(2,3-dihydroindole-1-carbonyl)pyrrolidin-1-yl]-8-propan-2-yl-3,5,8-triazabicyclo[4.3.0]nona-1,3,5-trien-7-one

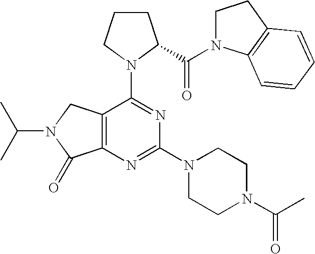

To a solution of (R)-1-(2-(4-acetylpiperazin-1-yl)-6-isopropyl-7-oxo-6,7-dihydro-5H-pyrrolo[3,4-d]pyrimidin-4-yl)pyrrolidine-2-carboxylic acid (Intermediate 82) (83 mg, 0.2 mmol) in DMA (3 mL) was added indoline (0.024 g, 0.20 mmol), HATU (0.091 g, 0.24 mmol) followed by DIPEA (0.042 mL, 0.24 mmol). The reaction mixture was stirred at rt for 3 h. The reaction mixture was concentrated under reduced pressure, and the residue was taken up into dichloromethane (15 mL), extracted with sat. NaHCO₃ (10 mL) and then brine (10 mL), dried over Na₂SO₄ and filtered. The filtrate was concentrated under reduced pressure, and the residue was purified with reverse-phase HPLC using high pH column (30-50% MeCN/H₂O) to give the title compound (33 mg, 32%) as a solid. HPLC purity>99% (215 nm), >97% (254 nm), >98% (280 nm); Rₜ: 1.465 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.10 (d, J=6.25 Hz, 6H), 1.21-1.28 (m, 4H), 1.80 (s, 3H), 1.91-2.10 (m, 2H), 2.10-2.25 (m, 1H), 2.31-2.43 (m, 1H), 2.82-2.95 (m, 1H), 2.95-3.12 (m, 2H), 3.29-3.41 (m, 1H), 3.45-3.59 (m, 2H), 3.60-3.71 (m, 1H), 3.78-3.93 (m, 2H), 4.12-4.22 (m, 1H), 4.31-4.50 (m, 2H), 4.58 (s, 2H), 6.99 (t, J=7.23 Hz, 1H), 7.09 (t, J=7.62 Hz, 1H), 7.22 (d, J=7.42 Hz, 1H), 8.00 (d, J=8.20 Hz, 1H). MS [M+H]⁺ 518.2 (ESI); HRMS m/z calcd for C₂₈H₃₆N₇O₃ [M+H] 518.28741, found: 518.28708.

### Example 280

### 4-(4-acetylpiperazin-1-yl)-2-[2-(3-phenyl-1,2,4-oxadiazol-5-yl)pyrrolidin-1-yl]-8-propan-2-yl-3,5,8-triazabicyclo[4.3.0]nona-1,3,5-trien-7-one

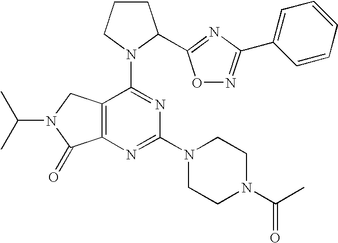

Following a procedure similar to that described in General Procedure 6 and after purification by preparative LCMS (high pH), the title compound was obtained as a solid (134 mg, 78%). HPLC purity>99% (215 nm), >99% (254 nm), >99% (280 nm); Rₜ: 1.882 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.07 (d, J=6.64 Hz, 2H), 1.31 (d, J=6.64 Hz, 6H), 1.92 (s, 3H), 2.08-2.29 (m, 2H), 2.31-2.43 (m, 1H), 2.46-2.56 (m, 1H), 3.12-3.24 (m, 1H), 3.33-3.39 (m, 1H), 3.44-3.56 (m, 1H), 3.54-3.74 (m, 3H), 3.92 (q, J=7.68 Hz, 1H), 4.05-4.16 (m, 1H), 4.45-4.55 (m, 1H), 4.58-4.74 (m, 2H), 5.48 (dd, J=8.01, 3.71 Hz, 1H), 7.42-7.58 (m, 3H), 7.99 (d, J=6.25 Hz, 2H). MS [M+H] 517.3 (ESI); HRMS m/z calcd for C₂₇H₃₃N₈O₃ [M+H] 517.26701, found: 517.26658.

### Example 281

### 4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one

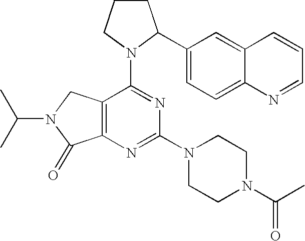

Following a procedure similar to that described in General Procedure 6 and after purification by preparative LCMS (high pH), the title compound was obtained as a solid (238 mg, 72%). HPLC purity>98% (215 nm), >93% (254 nm), >96% (280 nm); Rₜ: 1.097 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.11-1.52 (m, 6H), 1.79-2.27 (m, 6H), 2.42-2.59 (m, 1H), 2.79-3.22 (m, 3H), 3.34-3.83 (m, 5H), 3.91-4.06 (m, 1H), 4.13-4.23 (m, 1H), 4.41-4.61 (m, 1H), 4.64-4.78 (m, 2H), 5.41-5.56 (m, 1H), 7.48 (dd, J=8.20, 4.30 Hz, 1H), 7.62-7.78 (m, 2H), 7.98 (d, J=6.64 Hz, 1H), 8.29 (d, J=8.20 Hz, 1H), 8.77 (d, J=2.73 Hz, 1H). MS [M+H]⁺ 500.3 (ESI); HRMS m/z calcd for C₂₈H₃₄N₇O₂ [M+H]⁺ 500.27685, found: 500.27672.

### Example 282

### 4-(4-acetylpiperazin-1-yl)-8-propan-2-yl-2-(2-quinolin-3-ylpyrrolidin-1-yl)-3,5,8-triazabicyclo[4.3.0]nona-1,3,5-trien-7-one (Enantiomer 1) and

### Example 283

### 4-(4-acetylpiperazin-1-yl)-8-propan-2-yl-2-(2-quinolin-3-ylpyrrolidin-1-yl)-3,5,8-triazabicyclo[4.3.0]nona-1,3,5-trien-7-one (Enantiomer 2)

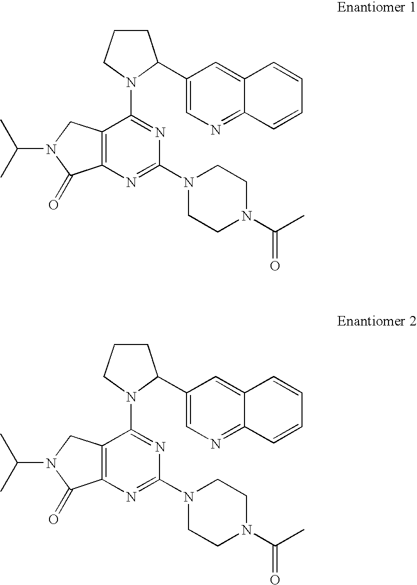

Following a procedure similar to that described in General Procedure 6 and after purification by preparative LCMS (high pH) followed by chiral HPLC (condition: Charial AD, 40% EtOH, 60% Heptane), both Enantiomer 1 (128 mg, 38.8%) and Enantiomer 2 (104 mg, 31.5%) were obtained.

Enantiomer 1: HPLC purity>99% (215 nm), >96% (254 nm), >98% (280 nm); Rₜ: 2.396 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.29-1.39 (m, 6H), 1.92 (s, 3H), 1.97-2.08 (m, 1H), 2.09-2.26 (m, 2H), 2.47-2.63 (m, 1H), 2.90-3.21 (m, 4H), 3.35-3.76 (m, 4H), 3.93-4.04 (m, 1H), 4.15-4.26 (m, 1H), 4.43-4.59 (m, 1H), 4.65-4.79 (m, 2H), 5.50 (d, J=3.52 Hz, 1H), 7.57 (t, J=7.42 Hz, 1H), 7.71 (t, J=7.23 Hz, 1H), 7.88 (d, J=7.81 Hz, 1H), 7.98 (d, J=8.20 Hz, 1H), 8.15 (s, 1H), 8.80 (s, 1H). MS [M+H]⁺ 500.2 (ESI); HRMS m/z calcd for C₂₈H₃₄N₇O₂ [M+H]⁺ 500.27654, found: 500.27685.

Enantiomer 2: HPLC purity>99% (215 nm), >97% (254 nm), >99% (280 nm); Rₜ: 2.404 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.29-1.39 (m, 6H), 1.92 (s, 3H), 1.98-2.07 (m, 1H), 2.08-2.26 (m, 2H), 2.49-2.62 (m, 1H) 2.99-3.20 (m, 4H), 3.31-3.65 (m, 4H), 3.99 (d, J=7.81 Hz, 1H), 4.17-4.27 (m, 1H), 4.40-4.59 (m, 1H), 4.64-4.79 (m, 2H), 5.50 (d, J=3.12 Hz, 1H), 7.57 (t, J=7.42 Hz, 1H), 7.71 (t, J=7.42 Hz, 1H), 7.87 (d, J=7.81 Hz, 1H), 7.98 (d, J=8.59 Hz, 1H), 8.15 (s, 1H), 8.80 (s, 1H). MS [M+H]⁺ 500.2 (ESI); HRMS m/z calcd for C₂₈H₃₄N₇O₂ [M+H]⁺ 500.27644, found: 500.27644.

### Example 284

### 4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one (Enantiomer 1) and

### Example 285

### 4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one (Enantiomer 2)

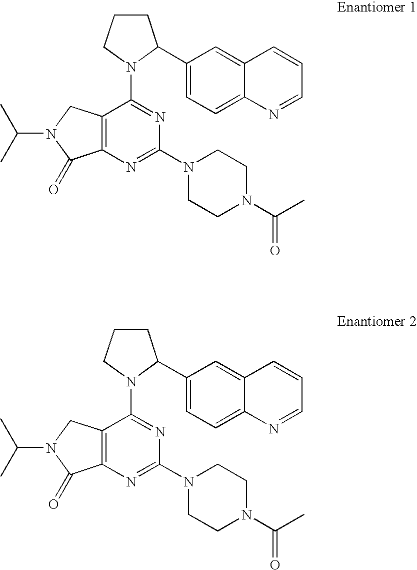

The racemic 4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one (0.300 g, 0.6 mmol, Example 281) was purified with semi-preparative SFC (condition: AS chiral ISO 40 EtOH), to yield Enantiomer 1 (0.173 g, 57.7%) and Enantiomer 2 (0.103 g, 34.4%).

Enantiomer 1: HPLC purity>99% (215 nm), >97% (254 nm), >98% (280 nm); Rₜ: 1.117 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.30 (d, J=6.25 Hz, 6H), 1.82-2.03 (m, 3H), 2.12 (s, 3H), 2.42-2.60 (m, 1H), 2.94-3.14 (m, 2H), 3.33-3.64 (m, 4H), 3.76-3.89 (m, 3H), 3.94-4.08 (m, 1H), 4.14-4.28 (m, 1H), 4.42-4.60 (m, 1H), 4.68-4.79 (m, 1H), 5.42-5.57 (m, 1H), 7.52 (dd, J=8.20, 4.30 Hz, 1H), 7.73 (d, J=8.59 Hz, 1H), 7.76-7.86 (m, 1H), 7.99 (d, J=7.03 Hz, 1H), 8.35 (d, J=8.20 Hz, 1H), 8.79 (s, 1H). MS [M+H]⁺ 500.3 (ESI); HRMS m/z calcd for C₂₈H₃₄N₇O₂ [M+H]⁺ 500.27685, found: 500.27766.

Enantiomer 2: HPLC purity>96% (215 nm), >95% (254 nm), >96% (280 nm); Rₜ: 1.119 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.20-1.50 (m, 6H), 1.80-2.27 (m, 6H), 2.41-2.57 (m, 1H), 2.82-3.22 (m, 3H), 3.37-3.82 (m, 6H), 3.92-4.04 (m, 1H), 4.13-4.26 (m, 1H), 4.42-4.59 (m, 1H), 4.66-4.80 (m, 1H), 5.42-5.56 (m, 1H), 7.48 (dd, J=8.20, 4.30 Hz, 1H), 7.62-7.79 (m, 2H), 7.98 (d, J=7.03 Hz, 1H), 8.29 (d, J=7.81 Hz, 1H), 8.77 (d, J=3.52 Hz, 1H). MS [M+H]⁺ 500.3 (ESI); HRMS m/z calcd for C₂₈H₃₄N₇O₂ [M+H]⁺ 500.27685, found: 500.27734.

### Example 286

### 2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one

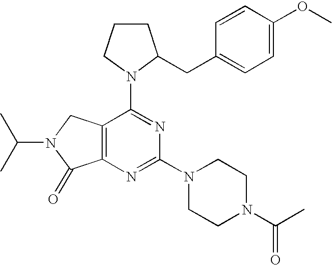

Following a procedure similar to that described in General Procedure 6 and after purification by preparative LCMS (high pH), the title compound was obtained as a solid (252 mg, 84%). HPLC purity>99% (215 nm), >99% (254 nm), >99% (280 nm); Rₜ: 1.795 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.29 (d, J=2.34 Hz, 6H), 1.74-1.99 (m, 4H), 2.12 (s, 3H), 2.63 (dd, J=13.09, 9.18 Hz, 1H), 3.00-3.18 (m, 1H), 3.57 (d, J=4.69 Hz, 2H), 3.62 (d, J=4.69 Hz, 2H), 3.64-3.71 (m, 1H), 3.73 (s, 3H), 3.79-3.95 (m, 4H), 4.35-4.71 (m, 4H), 6.82 (d, J=8.20 Hz, 2H), 7.08 (d, J=8.20 Hz, 2H). MS [M+H]⁺ 493.2 (ESI); HRMS m/z calcd for C₂₇H₃₇N₆O₃ [M+H]⁺ 493.29217, found: 493.29199.

### Example 287

### 2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one (Enantiomer 1) and

### Example 288

### 2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one (Enantiomer 2)

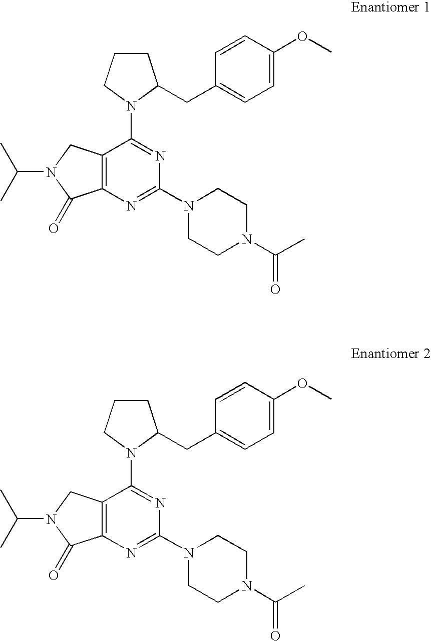

The racemic 2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one (230 mg, 0.47 mmol, Example 286) was purified with semi-preparative chiral HPLC (condition: chiral AD 20 iPrOH), to yield Enantiomer 1 (102 mg, 44.3%) and Enantiomer 2 (105 mg, 45.7%).

Enantiomer 1: HPLC purity>99% (215 nm), >99% (254 nm), >99% (280 nm); Rₜ: 1.787 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.25-1.33 (m, 6H), 1.78-1.99 (m, 4H), 2.12 (s, 3H), 2.64 (dd, J=13.28, 8.98 Hz, 1H), 3.11 (d, J=8.98 Hz, 1H), 3.53-3.64 (m, 4H), 3.64-3.71 (m, 1H), 3.70-3.79 (m, 2H), 3.73 (s, 2H), 3.79-3.86 (m, 2H), 3.89 (d, J=3.52 Hz, 2H), 4.33-4.67 (m, 4H), 6.82 (d, J=8.20 Hz, 2H), 7.08 (d, J=8.20 Hz, 2H). MS [M+H]⁺ 493.2 (ESI); HRMS m/z calcd for C₂₇H₃₇N₆O₃ [M+H]⁺ 493.29217, found: 493.29233.

Enantiomer 2: HPLC purity>96% (215 nm), >96% (254 nm), >97% (280 nm); Rₜ: 1.786 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.25-1.33 (m, 6H), 1.75-2.00 (m, 4H), 2.12 (s, 3H), 2.64 (dd, J=13.28, 8.98 Hz, 1H), 3.10 (s, 1H), 3.53-3.64 (m, 4H), 3.65-3.72 (m, 1H), 3.70-3.79 (m, 2H), 3.73 (s, 2H), 3.80-3.87 (m, 2H), 3.90 (d, J=3.52 Hz, 2H), 4.42-4.66 (m, 4H), 6.82 (d, J=8.20 Hz, 2H), 7.08 (d, J=8.20 Hz, 2H). MS [M+H]⁺ 493.2 (ESI); HRMS m/z calcd for C₂₇H₃₇N₆O₃ [M+H]⁺ 493.29217, found: 493.29154.

### Example 289

### 2-(4-acetylpiperazin-1-yl)-4-[2-[(4-ethoxyphenyl)methyl]pyrrolidin-1-yl]-6-isopropyl-5H-pyrrolo[4,3-e]pyrimidin-7-one

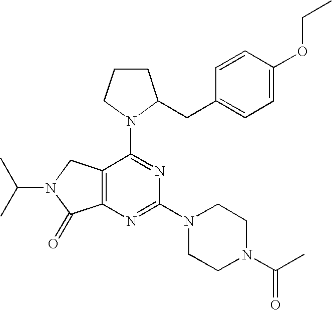

Following a procedure similar to that described in General Procedure 6, starting from 2-(4-ethoxybenzyl)pyrrolidine hydrochloride (Intermediate 77) and after purification by preparative LCMS (high pH), the title compound was obtained as a solid (56 mg, 28%). HPLC purity>96% (215 nm), >97% (254 nm), >96% (280 nm); Rₜ: 1.952 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.26-1.31 (m, 6H), 1.33 (t, J=7.03 Hz, 3H), 1.76-1.98 (m, 4H), 2.13 (s, 3H), 2.64 (dd, J=13.28, 8.98 Hz, 1H), 3.10 (s, 1H), 3.57 (t, J=5.08 Hz, 2H), 3.59-3.64 (m, 2H), 3.65-3.80 (m, 2H), 3.81-3.87 (m, 2H), 3.89 (d, J=3.52 Hz, 2H), 3.92-4.01 (m, 2H), 4.41-4.69 (m, 4H), 6.80 (d, J=7.81 Hz, 2H), 7.07 (d, J=8.20 Hz, 2H). MS [M+H]⁺ 507.2 (ESI); HRMS m/z calcd for C₂₈H₃₉N₆O₃ [M+H]⁺ 507.30782, found: 507.30859.

### Example 290

### 2-(4-acetylpiperazin-1-yl)-6-isopropyl-4-[2-[(2-methoxyphenyl)methyl]pyrrolidin-1-yl]-5H-pyrrolo[3,4-d]pyrimidin-7-one

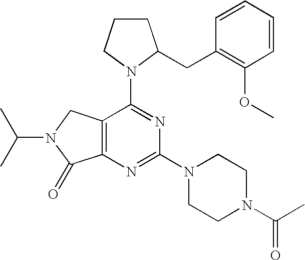

Following a procedure similar to that described in General Procedure 6, starting from 2-(2-methoxybenzyl)pyrrolidine hydrochloride (Intermediate 78) and after purification by preparative LCMS (high pH), the title compound was obtained as a solid (126 mg, 46%). HPLC purity>95% (215 nm), >95% (254 nm), >94% (280 nm); Rₜ: 1.794 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.29 (dd, J=6.25, 2.34 Hz, 6H), 1.63-1.86 (m, 2H), 1.89-1.98 (m, 1H), 1.99-2.08 (m, 1H), 2.12 (s, 3H), 2.70 (dd, J=13.09, 8.79 Hz, 1H), 3.55 (t, J=5.08 Hz, 2H), 3.59-3.64 (m, 2H), 3.64-3.73 (m, 1H), 3.77 (s, 3H), 3.78-3.82 (m, 2H), 3.80-3.87 (m, 2H), 3.87-3.92 (m, 2H), 4.42-4.78 (m, 4H), 6.83 (t, J=7.42 Hz, 1H), 6.89 (d, J=8.20 Hz, 1H), 7.09 (d, J=7.42 Hz, 1H), 7.16 (t, J=7.23 Hz, 1H). MS [M+H]⁺ 493.2 (ESI); HRMS [M+H]⁺ calc. for C₂₇H₃₇N₆O₃=493.29217, [M+H]⁺ obs.=493.29181.

### Example 291

### 2-(4-acetylpiperazin-1-yl)-6-isopropyl-4-[2-[(3-methoxyphenyl)methyl]pyrrolidin-1-yl]-5H-pyrrolo[3,4-d]pyrimidin-7-one

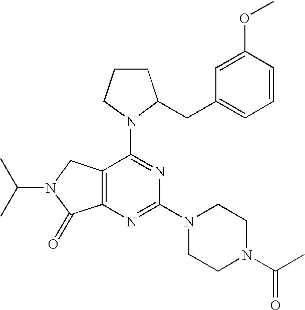

Following a procedure similar to that described in General Procedure 6, starting from 2-(3-methoxybenzyl)pyrrolidine hydrochloride (Intermediate 79) and after purification by preparative LCMS (high pH), the title compound was obtained as a solid (0.128 g, 52.0%). HPLC purity>99% (215 nm), >98% (254 nm), >98% (280 nm); Rₜ: 1.734 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.29 (d, J=6.25 Hz, 6H), 1.79-1.88 (m, 2H), 1.91-2.02 (m, 2H), 2.12 (s, 3H), 2.65 (dd, J=12.89, 8.98 Hz, 1H), 3.54-3.59 (m, 2H), 3.59-3.63 (m, 2H), 3.64-3.71 (m, 1H), 3.72 (s, 3H), 3.75-3.81 (m, 2H), 3.82-3.86 (m, 2H), 3.88-3.94 (m, 2H), 4.40-4.70 (m, 4H), 6.68-6.80 (m, 3H), 7.11-7.20 (m, 1H). MS [M+H]⁺ 493.2 (ESI); HRMS [M+H]⁺ calc. for C₂₇H₃₇N₆O₃=493.29217, [M+H]⁺ obs.=493.29210.

### Example 292

### 2-(4-acetylpiperazin-1-yl)-4-[2-(4-fluorophenyl)pyrrolidin-1-yl]-6-(1-methylethyl)-5,6-dihydro-7H-pyrrolo[3,4-d]pyrimidin-7-one

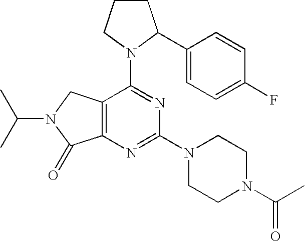

Following a procedure similar to that described in General Procedure 6 and after purification by preparative LCMS (high pH), the title compound was obtained as a solid (97 mg, 50.7%). ¹H NMR (400 MHz, CD₃OD) [symbol] ppm 1.27 (br. s., 6H), 1.89 (br. s., 2H), 1.95-2.05 (m, 2H), 2.07 (br.s., 3H), 2.40 (br. s., 1H), 3.63 (s, 8H), 3.87 (br. s., 1H), 4.01-4.13 (m, 1H), 4.47 (br. s., 1H), 4.63 (br. s., 1H), 5.30 (d, J=7.42 Hz, 1H), 7.02 (d, J=7.81 Hz, 2H), 7.21 (dd, J=8.40, 5.27 Hz, 2H). ES [M+H]⁺=467.3; HRMS [M+H]⁺ calc. for C₂₅H₃₃FN₆O₂=467.25653, [M+H]⁺ obs.=467.25641.

### Example 293

### 2-(4-acetylpiperazin-1-yl)-4-{2-[4-(methoxymethyl)benzyl]pyrrolidin-1-yl}-6-(1-methylethyl)-5,6-dihydro-7H-pyrrolo[3,4-d]pyrimidin-7-one

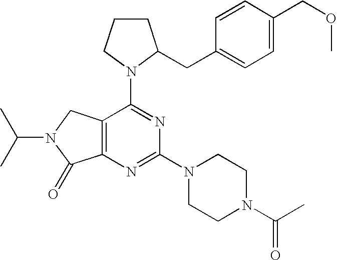

Following a procedure similar to that described in General Procedure 6, starting from 2-(4-(methoxymethyl)benzyl)pyrrolidine hydrochloride (Intermediate 80) and after purification by preparative LCMS (high pH), the title compound was obtained as a solid (39 mg, 48.1%). ¹H NMR (400 MHz, CD₃OD) [symbol] ppm 1.29 (dd, J=6.64, 3.12 Hz, 6H), 1.76-1.89 (m, 2H), 1.91-2.01 (m, 2H), 2.13 (s, 3H), 3.32 (s, 3H), 3.47-3.70 (m, 7H), 3.71-3.81 (m, 2H), 3.82-3.93 (m, 4H), 4.39 (s, 2H), 4.45-4.79 (m, 3H), 7.15-7.31 (m, 4H). ES [M+H]⁺=507.2; HRMS [M+H]⁺ calc. for C₂₈H₃₉N₆O₃=507.30782, [M+H]⁺ obs.=507.30750.

### Example 294

### 4-[2-(4-acetylbenzyl)pyrrolidin-1-yl]-2-(4-acetylpiperazin-1-yl)-6-(1-methylethyl)-5,6-dihydro-7H-pyrrolo[3,4-d]pyrimidin-7-one

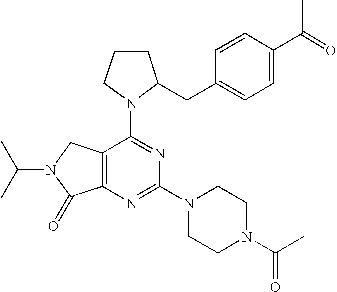

Following a procedure similar to that described in General Procedure 6, starting from 1-(4-(pyrrolidin-2-ylmethyl)phenyl)ethanone hydrochloride (Intermediate 81) and after purification by preparative LCMS (high pH), the title compound was obtained as a solid (16 mg, 19.8%). ¹H NMR (400 MHz, CD₃OD) [symbol] ppm 1.27-1.33 (m, 6H), 1.76-2.00 (m, 4H), 2.13 (s, 3H), 2.56 (s, 3H), 3.57 (t, J=5.08 Hz, 3H), 3.60-3.64 (m, 3H), 3.68 (q, J=8.72 Hz, 1H), 3.74-3.81 (m, 2H), 3.81-3.86 (m, 2H), 3.89 (d, J=3.91 Hz, 2H), 4.40-4.73 (m, 3H), 7.34 (d, J=7.81 Hz, 2H), 7.91 (d, J=7.81 Hz, 2H). ES [M+H]⁺=505.2; HRMS [M+H]⁺ calc. for C₂₈H₃₇N₆O₃=505.29217, [M+H]⁺ obs.=505.29212.

### Example 295

### 2-(4-acetylpiperazin-1-yl)-4-[2-(4-methoxy-3-methylphenyl)pyrrolidin-1-yl]-6-(1-methylethyl)-5,6-dihydro-7H-pyrrolo[3,4-d]pyrimidin-7-one

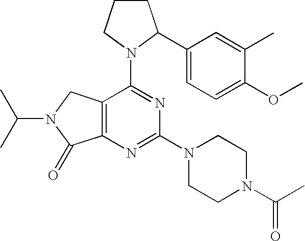

Following a procedure similar to that described in General Procedure 6 and after purification by preparative LCMS (high pH), the title compound was obtained as a solid (92 mg, 37.4%). ¹H NMR (400 MHz, CD₃OD) [symbol] ppm 0.74-1.46 (m, 6H), 1.81-2.03 (m, 4H), 2.08 (br. s., 3H), 2.13 (s, 3H), 2.37 (br. s., 1H), 3.36-3.72 (m, 7H), 3.75 (s, 3H), 3.79-3.97 (m, 2H), 4.05 (br. s., 1H), 4.26-4.80 (m, 2H), 5.21 (d, J=7.42 Hz, 1H), 6.80 (d, J=7.81 Hz, 1H), 6.93 (d, J=8.98 Hz, 1H), 6.95 (s, 1H). ES [M+H]⁺=493.2; HRMS [M+H]⁺ calc. for C₂₇H₃₇N₆O₃=493.29217, [M+H]⁺ obs.=493.29245.

### Example 296

### 2-(4-acetylpiperazin-1-yl)-6-(1-methylethyl)-4-[2-(4-methylphenyl)pyrrolidin-1-yl]-5,6-dihydro-7H-pyrrolo[3,4-d]pyrimidin-7-one

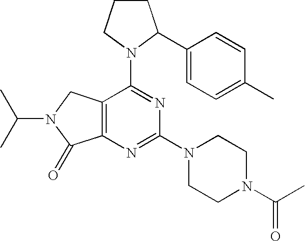

Following a procedure similar to that described in General Procedure 6 and after purification by preparative LCMS (high pH), the title compound was obtained as a solid (98 mg, 42.4%). HPLC purity: >99% (215 nm), >99% (254 nm), >99% (280 nm); Rₜ: 1.780 minutes. ¹H NMR (400 MHz, CD₃OD) [symbol] ppm 1.13-1.43 (m, 6H), 1.80-1.94 (m, 1H), 1.94-2.06 (m, 2H), 2.07 (br. s., 3H), 2.26 (s, 3H), 2.32-2.49 (m, 1H), 3.33-3.70 (m, 6H), 3.64-3.95 (m, 5H), 3.97-4.16 (m, 1H), 4.26-4.76 (m, 1H), 5.26 (d, J=7.03 Hz, 1H), 6.84-7.30 (m, 4H). M.S. (found): 463.3 (ESI) (MH⁺); HRMS m/z calcd for C₂₆H₃₅N₇O₂ [M+H]⁺ 463.28160, found 463.28157.

### Example 297

### 2-(4-acetylpiperazin-1-yl)-4-[2-(3,4-dichlorophenyl)pyrrolidin-1-yl]-6-(1-methylethyl)-5,6-dihydro-7H-pyrrolo[3,4-d]pyrimidin-7-one

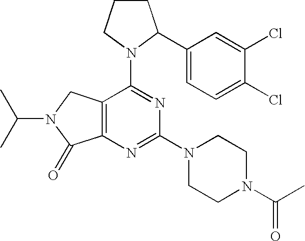

Following a procedure similar to that described in General Procedure 6 and after purification by preparative LCMS (high pH), the title compound was obtained as a solid (112 mg, 43.3%). HPLC purity: 99% (215 nm), >99% (254 nm), 99% (280 nm); Rₜ: 1.970 minutes. ¹H NMR (400 MHz, CD₃OD) [symbol] ppm 1.30 (d, J=5.08 Hz, 6H), 1.87 (br. s., 1H), 2.06 (s, 3H), 2.07-2.19 (m, 2H), 2.42 (dd, J=12.11, 8.20 Hz, 1H), 3.29-3.48 (m, 5H), 3.49-3.77 (m, 4H), 3.89 (d, J=7.81 Hz, 1H), 4.02-4.15 (m, 1H), 4.50 (br. s., 1H), 4.66 (br. s., 1H), 5.23 (d, J=4.30 Hz, 1H), 7.15 (dd, J=8.20, 1.56 Hz, 1H), 7.35-7.47 (m, 2H). M.S. (found): 517.3 (ESI) (MH⁺); HRMS m/z calcd for C₂₅H₃₁Cl₂N₆O₂ [M+H]⁺ 517.18801, found 517.18723.

### Example 298

### 2-(4-acetylpiperazin-1-yl)-4-[2-(3,4-dimethylphenyl)pyrrolidin-1-yl]-6-(1-methylethyl)-5,6-dihydro-7H-pyrrolo[3,4-d]pyrimidin-7-one

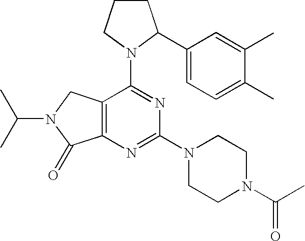

Following a procedure similar to that described in General Procedure 6 and after purification by preparative LCMS (high pH), the title compound was obtained as a solid (108 mg, 45.3%). HPLC purity: 100% (215 nm), >99% (254 nm), 100% (280 nm); Rₜ: 1.921 minutes. ¹H NMR (400 MHz, CD₃OD) [symbol] ppm 1.15-1.35 (m, 6H), 1.75-2.04 (m, 5H), 2.08 (br. s., 3H), 2.19 (s, 3H), 2.21 (s, 3H), 2.38 (br. s., 1H), 3.39-3.95 (m, 10H), 4.05 (br. s., 1H), 4.24-4.72 (m, 2H), 5.21 (d, J=6.25 Hz, 1H), 6.86 (d, J=7.81 Hz, 1H), 6.94 (s, 1H), 6.99-7.08 (m, 1H). M.S. (found): 477.2 (ESI) (MH⁺); HRMS m/z calcd for C₂₇H₃₇N₆O₂ [M+H]⁺ 477.29725, found 477.29706.

### Example 299

### 2-(4-acetylpiperazin-1-yl)-4-[2-(4-methoxyphenyl)pyrrolidin-1-yl]-6-(1-methylethyl)-5,6-dihydro-7H-pyrrolo[3,4-d]pyrimidin-7-one

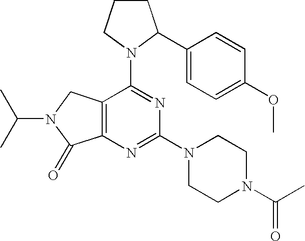

Following a procedure similar to that described in General Procedure 6 and after purification by preparative LCMS (high pH), the title compound was obtained as a solid (112 mg, 46.8%). HPLC purity: 100% (215 nm), >99% (254 nm), 100% (280 nm); Rₜ: 1.617 minutes. ¹H NMR (400 MHz, CD₃OD) [symbol] ppm 1.25 (br. s., 6H), 1.72-2.05 (m, 3H), 2.08 (s, 3H), 2.28 (br. s., 1H), 3.38-3.66 (m, 9H), 3.73 (s, 3H), 3.77-3.94 (m, 2H), 4.05 (d, J=5.08 Hz, 1H), 4.32-4.76 (m, 1H), 5.25 (d, J=6.25 Hz, 1H), 6.85 (d, J=7.42 Hz, 2H), 7.09 (d, J=8.59 Hz, 2H). M.S. (found): 479.2 (ESI) (MH⁺); HRMS m/z calcd for C₂₇H₃₅N₆O₃ [M+H]⁺ 479.27652, found 479.27614.

### Example 300

### 2-(4-acetylpiperazin-1-yl)-4-[(2R)-2-(4-ethoxy-3-fluorophenyl)pyrrolidin-1-yl]-6-(1-methylethyl)-5,6-dihydro-7H-pyrrolo[3,4-d]pyrimidin-7-one

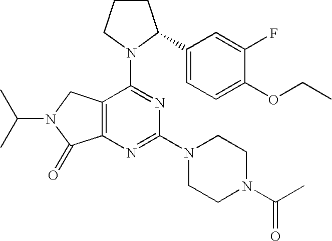

To a solution of crude 2-(4-acetylpiperazin-1-yl)-4-[(2R)-2-(4-hydroxy-3-fluorophenyl)pyrrolidin-1-yl]-6-(1-methylethyl)-5,6-dihydro-7H-pyrrolo[3,4-d]pyrimidin-7-one (Intermediate 124) (0.121 g, 0.25 mmol) in DMF (5 mL) was added K₂CO₃ (69 mg, 0.5 mmol) followed by iodoethane at rt. The reaction mixture was stirred for 18 h at 50° C. Water was added and extracted with EtOAc (2×). The organic layer was dried (Na₂SO₄), filtered and concentrated under reduced pressure. The residue was purified by preparative LCMS (high pH) to give the title compound (52 mg, 40.6%) as a solid. HPLC purity: >90% (215 nm), >87% (254 nm), >93% (280 nm); Rₜ: 1.809 minutes. ¹H NMR (400 MHz, CD₃OD) [symbol] ppm 1.20-1.34 (m, 6H), 1.34 (t, J=7.03 Hz, 3H), 1.88 (br. s., 1H), 1.97-2.07 (m, 2H), 2.07 (s, 3H), 2.37 (br. s., 1H), 3.36-3.94 (m, 9H), 4.04 (q, J=7.03 Hz, 2H), 4.02-4.10 (m, 2H), 4.42-4.73 (m, 2H), 5.23 (d, J=7.03 Hz, 1H), 6.85-7.04 (m, 3H). M.S. (found): 511.2 (ESI) (MH⁺). HRMS m/z calcd for C₂₇H₃₆FN₆O₃ [M+H]⁺ 511.28274, found 511.28270.

### Example 301

### (4-chlorophenyl)methyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate

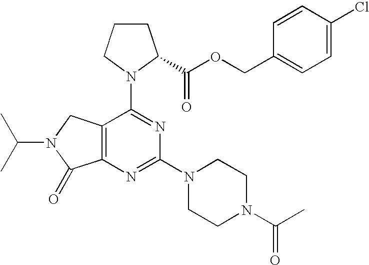

Following a procedure similar to that described in Example 279 and after purification by reverse-phase HPLC using high pH column (30-50% MeCN/H₂O), the title compound (21 mg, 19.4%) was obtained as a solid. HPLC purity>99% (215 nm), >99% (254 nm), >99% (280 nm); Rₜ: 1.886 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.30 (d, J=6.25 Hz, 6H), 2.07 (s, 3H), 2.06-2.10 (m, 3H), 2.25-2.38 (m, 1H), 3.33-3.51 (m, 5H), 3.53-3.71 (m, 3H), 3.78-3.86 (m, 1H), 3.87-3.96 (m, 1H), 4.46-4.56 (m, 1H), 4.58-4.67 (m, 3H), 5.02 (d, J=12.50 Hz, 1H), 5.21 (d, J=12.50 Hz, 1H), 7.16-7.22 (m, 2H), 7.24-7.30 (m, 2H). MS [M+H]⁺ 541.3 (ESI); HRMS m/z calcd for C₂₇H₃₄ClN₆O₄ [M+H]⁺ 541.23246, found 541.23180.

### Example 302

### (2R)-1-[4-(4-acetylpiperazin-1-yl)-7-oxo-8-propan-2-yl-3,5,8-triazabicyclo[4.3.0]nona-1,3,5-trien-2-yl]-N-cyclohexyl-pyrrolidine-2-carboxamide

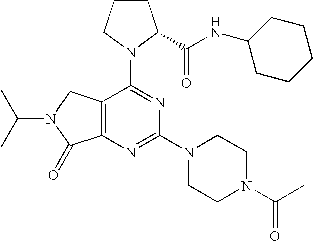

Following a procedure similar to that described in Example 279 and after purification by reverse-phase HPLC using high pH column (30-50% MeCN/H₂O), the title compound was obtained as a solid (63 mg, 63.3%). HPLC purity>99% (215 nm), >99% (254 nm), >99% (280 nm); Rₜ: 1.431 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.03-1.26 (m, 4H), 1.30 (d, J=6.64 Hz, 6H), 1.35-1.51 (m, 2H), 1.61 (d, J=12.50 Hz, 1H), 1.67-1.85 (m, 4H), 1.90-2.07 (m, 2H), 2.11 (s, 3H), 2.19-2.34 (m, 1H), 3.52 (t, J=4.88 Hz, 2H), 3.55-3.67 (m, 3H), 3.69-3.87 (m, 5H), 3.88-3.98 (m, 1H), 4.46-4.56 (m, 2H), 4.58-4.69 (m, 2H). MS [M+H]⁺ 498.2 (ESI); HRMS m/z calcd for C₂₆H₄₀N₇O₃ [M+H]⁺ 498.31871, found 498.31832.

### Example 303

### 4-(4-acetylpiperazin-1-yl)-2-[(2R)-2-(4-methylpiperidine-1-carbonyl)pyrrolidin-1-yl]-8-propan-2-yl-3,5,8-triazabicyclo[4.3.0]nona-2,4,10-trien-7-one

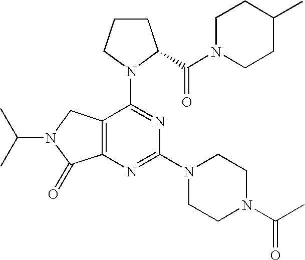

Following a procedure similar to that described in Example 279 and after purification by reverse-phase HPLC using high pH column (30-50% MeCN/H₂O), the title compound was obtained as a solid (74 mg, 74.4%). HPLC purity>99% (215 nm), >99% (254 nm), >99% (280 nm); Rₜ: 1.430 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.09 (d, J=6.64 Hz, 3H), 1.30 (d, J=6.64 Hz, 6H), 1.65-1.82 (m, 2H), 1.79-1.97 (m, 2H), 2.01-2.14 (m, 2H), 2.11 (s, 3H), 2.23-2.39 (m, 1H), 2.58-2.72 (m, 1H), 3.05-3.24 (m, 1H), 3.41-3.62 (m, 5H), 3.63-3.77 (m, 3H), 3.79-3.94 (m, 4H), 4.07-4.25 (m, 1H), 4.35-4.57 (m, 2H), 4.57-4.70 (m, 2H), 5.05-5.20 (m, 1H). MS [M+H] 498.2 (ESI); HRMS m/z calcd for C₂₆H₄₀N₇O₃ [M+H] 498.31871, found 498.318712.

### Example 304

### phenyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate

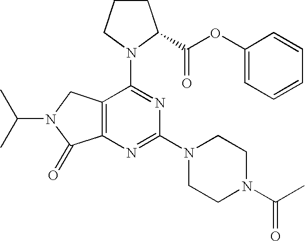

Following a procedure similar to that described in Example 279 and after purification by reverse-phase HPLC using high pH column (30-50% MeCN/H₂O), the title compound was obtained as a solid (21 mg, 21%). HPLC purity>92% (215 nm), >92% (254 nm), >92% (280 nm); Rₜ: 1.717 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.31 (d, J=5.08 Hz, 6H), 2.07 (s, 3H), 2.15-2.33 (m, 3H), 2.42-2.55 (m, 1H), 3.39-3.59 (m, 4H), 3.66-3.85 (m, 4H), 3.86-3.94 (m, 1H), 3.95-4.02 (m, 1H), 4.46-4.58 (m, 1H), 4.58-4.74 (m, 2H), 4.77 (d, J=4.69 Hz, 1H), 6.98 (d, J=7.81 Hz, 2H), 7.21 (t, J=7.42 Hz, 1H), 7.36 (t, J=7.81 Hz, 2H). MS [M+H]⁺ 493.2 (ESI).

### Example 305

### 3-(4-acetylpiperazin-1-yl)-5-[[1-(4-fluorophenyl)-2-methyl-propan-2-yl]amino]-8-(oxan-4-yl)-2,4,8-triazabicyclo[4.3.0]nona-1,3,5-trien-9-one

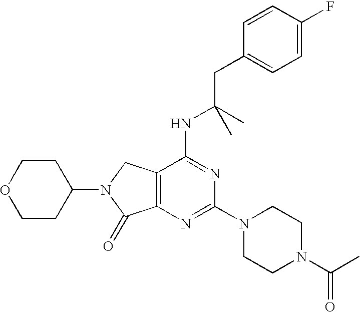

A solution of 2,4-dichloro-6-tetrahydropyran-4-yl-5H-pyrrolo[3,4-d]pyrimidin-7-one (Intermediate 17) (189 mg, 0.66 mmol), 1-(4-fluorophenyl)-2-methylpropan-2-amine (110 mg, 0.66 mmol) and DIPEA (0.229 mL, 1.31 mmol) in THF (4 mL) was heated in a microwave reactor at 140° C. for 30 minutes, cooled to rt and concentrated under reduced pressure. The residue was dissolved in n-BuOH (4.00 mL) followed by addition of 1-(piperazin-1-yl)ethanone (46.9 mg, 0.37 mmol) and DIPEA (0.106 mL, 0.61 mmol) and heated in a microwave reactor at 160° C. for 30 minutes. The title compound was obtained as a solid (35.0 mg, 21.6%) following purification by silica gel chromatography (gradient 10-20% MeOH in CH₂Cl₂), preparative LCMS (gradient 35-55% CH₃CN in H₂O containing 10 mM NH₄HCO₃) and lyophilization from CH₃CN/H₂O. HPLC purity>99% (215 nm), >99% (254 nm), >99% (280 nm); Rₜ: 1.68 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.45 (s, 6H), 1.71-1.92 (m, 4H), 2.14 (s, 3H), 3.31 (s, 2H), 3.55 (td, J=11.82, 2.15 Hz, 2H), 3.60-3.69 (m, 4H), 3.85-3.90 (m, 2H), 3.94 (dd, J=6.25, 4.30 Hz, 2H), 4.02 (dd, J=11.33, 4.30 Hz, 2H), 4.12 (s, 2H), 4.28-4.42 (m, 1H), 6.86-6.96 (m, 2H), 6.99-7.07 (m, 2H). MS [M+H]⁺ 511.2 (ESI). HRMS m/z calcd for C₂₇H₃₆FN₆O₃ [M+H]⁺ 511.2827, found 511.2826.

### Example 306

### (+)-4-(4-acetylpiperazin-1-yl)-8-butan-2-yl-2-[(1-phenylpyrazol-4-yl)methylamino]-3,5,8-triazabicyclo[4.3.0]nona-1,3,5-trien-7-one (Enantiomer 1) and

### Example 307

### (−)-4-(4-acetylpiperazin-1-yl)-8-butan-2-yl-2-[(1-phenylpyrazol-4-yl)methylamino]-3,5,8-triazabicyclo[4.3.0]nona-1,3,5-trien-7-one (Enantiomer 2)

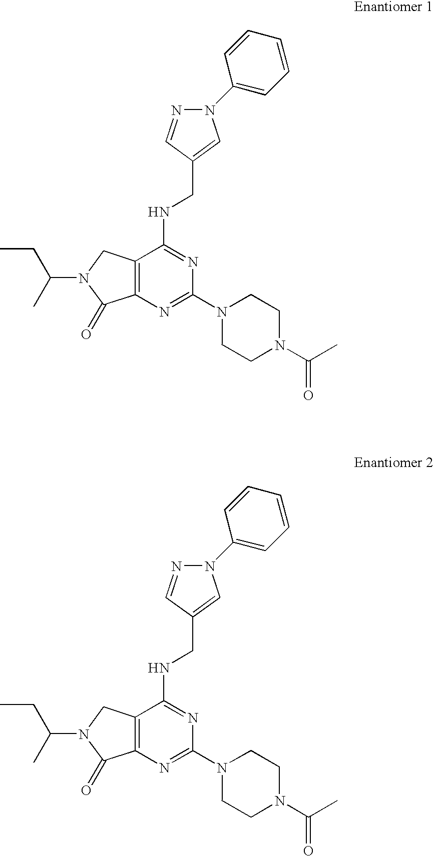

Following a procedure similar to that described in General Procedure 1, starting from 6-sec-butyl-2,4-dichloro-5H-pyrrolo[3,4-d]pyrimidin-7(6H)-one (Intermediate 18) and after purification by preparative LCMS (gradient 35-55% CH₃CN in H₂O containing 10 mM NH₄HCO₃) followed by lyophilisation from CH₃CN/H₂O, the mixture of two enantiomers was obtained which was separated by SFC (AS column with EtOH+0.1% DMEA, Iso at 40%) to provide (+)-2-(4-acetylpiperazin-1-yl)-6-sec-butyl-4-((1-phenyl-1H-pyrazol-4-yl)methylamino)-5H-pyrrolo[3,4-d]pyrimidin-7(6H)-one (69.0 mg, 13.12%) as a solid and (−)-2-(4-acetylpiperazin-1-yl)-6-sec-butyl-4-((1-phenyl-1H-pyrazol-4-yl)methylamino)-5H-pyrrolo[3,4-d]pyrimidin-7(6H)-one (61.0 mg, 11.60%) as a solid following lyophilization from CH₃CN/H₂O.

Enantiomer 1: HPLC purity: >99% (215 nm), >97% (254 nm), >97% (280 nm); Rₜ: 1.63 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} +12.80 (c=0.01, MeOH at 25° C.), 99% ee. ¹H NMR (400 MHz, CD₃OD) δ ppm 0.82 (t, J=7.42 Hz, 3H), 1.23 (d, J=6.64 Hz, 3H), 1.61 (t, J=7.42 Hz, 2H), 2.08 (s, 3H), 3.49-3.55 (m, 2H), 3.54-3.61 (m, 2H), 3.77-3.83 (m, 2H), 3.83-3.92 (m, 2H), 4.03-4.19 (m, 2H), 4.25 (d, J=7.03 Hz, 1H), 4.60 (s, 2H), 7.27 (d, J=7.42 Hz, 1H), 7.42 (t, J=8.01 Hz, 2H), 7.59-7.72 (m, 3H), 8.16 (s, 1H). MS [M+H]⁺ 489.2 (ESI). HRMS m/z calcd for C₂₆H₃₃N₈O₂ [M+H]⁺ 489.2721, found 489.2717.

Enantiomer 2: HPLC purity: >99% (215 nm), >99% (254 nm), >94% (280 nm); Rₜ: 1.63 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} −11.90 (c=0.01, MeOH at 25° C.), 98.5% ee. ¹H NMR (400 MHz, CD₃OD) δ ppm 0.86 (t, J=7.42 Hz, 3H), 1.27 (d, J=7.03 Hz, 3H), 1.54-1.73 (m, 2H), 2.11 (s, 3H), 3.50-3.65 (m, 4H), 3.81-3.88 (m, 2H), 3.87-3.94 (m, 2H), 4.04-4.25 (m, 2H), 4.29 (q, J=7.16 Hz, 1H), 4.64 (s, 2H), 7.30 (t, J=7.62 Hz, 1H), 7.39-7.51 (m, 2H), 7.61-7.76 (m, 3H), 8.19 (s, 1H). MS [M+H]⁺ 489.2 (ESI). HRMS m/z calcd for C₂₆H₃₃N₈O₂ [M+H]⁺ 489.2721, found 489.2718.

### Example 308

### (+)-2-(4-acetylpiperazin-1-yl)-4-(3-quinolylmethylamino)-6-sec-butyl-5H-pyrrolo[3,4-d]pyrimidin-7-one (Enantiomer 1) and

### Example 309

### (−)-2-(4-acetylpiperazin-1-yl)-4-(3-quinolylmethylamino)-6-sec-butyl-5H-pyrrolo[3,4-d]pyrimidin-7-one (Enantiomer 2)

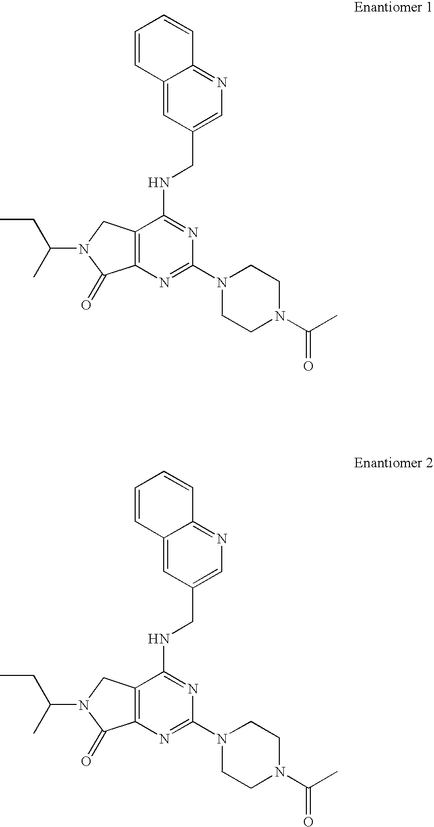

Following a procedure similar to that described in General Procedure 1, starting from 6-sec-butyl-2,4-dichloro-5H-pyrrolo[3,4-d]pyrimidin-7(6H)-one (Intermediate 18) and after purification by preparative LCMS (gradient 25-45% CH₃CN in H₂O containing 10 mM NH₄HCO₃) followed by lyophilisation from CH₃CN/H₂O, the mixture of two enantiomers was obtained which was separated by SFC (AS column with EtOH+0.1% DMEA, Iso at 40%) to provide Enantiomer 1: (+)-2-(4-acetylpiperazin-1-yl)-6-sec-butyl-4-(quinolin-3-ylmethylamino)-5H-pyrrolo[3,4-d]pyrimidin-7(6H)-one (61.0 mg, 11.17%) as a solid and Enantiomer 2: (−)-2-(4-acetylpiperazin-1-yl)-6-sec-butyl-4-(quinolin-3-ylmethylamino)-5H-pyrrolo[3,4-d]pyrimidin-7(6H)-one (4.00 mg, 0.732%) as a solid following lyophilisation from CH₃CN/H₂O.

Enantiomer 1: HPLC purity: >96% (215 nm), >98% (254 nm), >97% (280 nm); Rₜ: 1.13 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} +18.4 (c=0.01, MeOH at 25° C.), 99% (ee). ¹H NMR (400 MHz, CD₃OD) δ ppm 0.88 (t, J=7.42 Hz, 3H), 1.29 (d, J=6.64 Hz, 3H), 1.66 (q, J=7.42 Hz, 2H), 2.08 (s, 3H), 3.40-3.45 (m, 2H), 3.47-3.53 (m, 2H), 3.72-3.78 (m, 2H), 3.78-3.84 (m, 2H), 4.13-4.34 (m, 3H), 4.90 (s, 2H), 7.60 (t, J=7.03 Hz, 1H), 7.71-7.77 (m, 1H), 7.92 (d, J=8.59 Hz, 1H), 8.01 (d, J=8.20 Hz, 1H), 8.32 (s, 1H), 8.90 (d, J=1.95 Hz, 1H). MS [M+H]⁺ 474.2 (ESI). HRMS m/z calc for C₂₆H₃₂N₇O₂ [M+H]⁺ 474.2612, found 474.2609.

Enantiomer 2: HPLC purity: >96% (215 nm), >97% (254 nm), >95% (280 nm); Rₜ: 1.14 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} −3.3 (c=0.04, MeOH), 90% (ee). ¹H NMR (400 MHz, CD₃OD) δ ppm 0.88 (t, J=7.42 Hz, 3H), 1.29 (d, J=6.64 Hz, 3H), 1.66 (q, J=7.42 Hz, 2H), 2.08 (s, 3H), 3.39-3.53 (m, 4H), 3.70-3.85 (m, 4H), 4.13-4.36 (m, 3H), 4.90 (s, 2H), 7.60 (t, J=7.42 Hz, 1H), 7.69-7.79 (m, 1H), 7.92 (d, J=8.20 Hz, 1H), 8.01 (d, J=8.59 Hz, 1H), 8.32 (s, 1H), 8.90 (d, J=1.56 Hz, 1H). MS [M+H] 474.2 (ESI). HRMS m/z calcd for C₂₆H₃₂N₇O₂ [M+H]⁺ 474.2612, found 474.2613.

### Example 310

### (+)-2-(4-acetylpiperazin-1-yl)-4-(3-isoquinolylmethylamino)-6-sec-butyl-5H-pyrrolo[3,4-d]pyrimidin-7-one (Enantiomer 1) and

### Example 311

### (−)-2-(4-acetylpiperazin-1-yl)-4-(3-isoquinolylmethylamino)-6-sec-butyl-5H-pyrrolo[3,4-d]pyrimidin-7-one (Enantiomer 2)

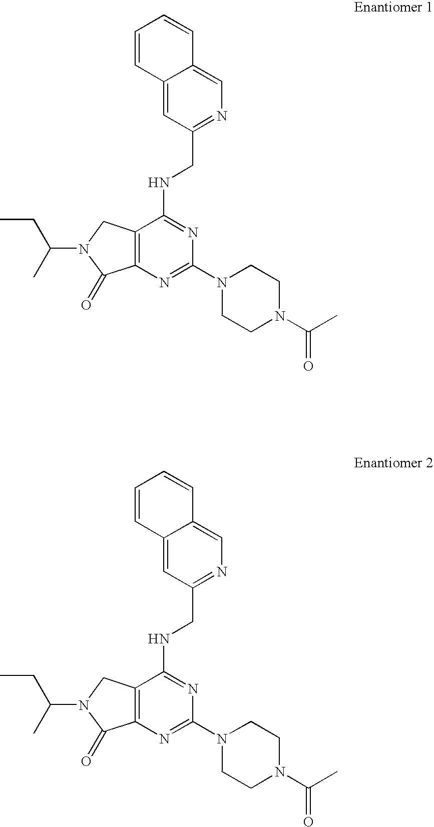

Following a procedure similar to that described in General Procedure 1, starting from 6-sec-butyl-2,4-dichloro-5H-pyrrolo[3,4-d]pyrimidin-7(6H)-one (Intermediate 18) and after purification by preparative LCMS (gradient 25-45% CH₃CN in H₂O containing 10 mM NH₄HCO₃) followed by lyophilisation from CH₃CN/H₂O, the mixture of two enantiomers was obtained which was separated by SFC (AS column with EtOH+0.1% DMEA, Iso at 40%) to provide (+)-2-(4-acetylpiperazin-1-yl)-6-sec-butyl-4-(isoquinolin-3-ylmethylamino)-5H-pyrrolo[3,4-d]pyrimidin-7(6H)-one (70.0 mg, 5.86%) as a solid and (−)-2-(4-acetylpiperazin-1-yl)-6-sec-butyl-4-(isoquinolin-3-ylmethylamino)-5H-pyrrolo[3,4-d]pyrimidin-7(6H)-one (38.00 mg, 6.96%) as a solid following lyophilization from CH₃CN/H₂O.

Enantiomer 1: HPLC purity: >96% (215 nm), >96% (254 nm), >92% (280 nm); Rₜ: 1.22 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} +11.6 (c=0.1, MeOH), 99% ee. MS [M+H]⁺ 474.2 (ESI). ¹H NMR (400 MHz, CD₃OD) δ ppm 0.90 (t, J=7.42 Hz, 3H), 1.31 (d, J=6.64 Hz, 3H), 1.69 (q, J=7.55 Hz, 2H), 2.04 (s, 3H), 3.33-3.37 (m, 2H), 3.38-3.45 (m, 2H), 3.64-3.70 (m, 2H), 3.74 (d, J=10.16 Hz, 2H), 4.17-4.37 (m, 3H), 4.92 (s, 2H), 7.64 (t, J=7.62 Hz, 1H), 7.75 (t, J=8.20 Hz, 1H), 7.80 (s, 1H), 7.88 (d, J=7.42 Hz, 1H), 8.09 (d, J=8.20 Hz, 1H), 9.21 (s, 1H). MS [M+H]⁺ 474.2 (ESI). HRMS m/z calcd for C₂₆H₃₂N₇O₂ [M+H]⁺ 474.2612, found 474.2612.

Enantiomer 2: HPLC purity: >97% (215 nm), >97% (254 nm), >95% (280 nm); Rₜ: 1.22 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} −12.0 (c=0.1, MeOH), 99% ee. MS [M+H]⁺ 474.2 (ESI). ¹H NMR (400 MHz, CD₃OD) δ ppm 0.90 (t, J=7.42 Hz, 3H), 1.31 (d, J=7.03 Hz, 3H), 1.68 (q, J=7.03 Hz, 2H), 2.04 (s, 3H), 3.32-3.38 (m, 2H), 3.39-3.44 (m, 2H), 3.64-3.70 (m, 2H), 3.70-3.76 (m, 2H), 4.18-4.38 (m, 3H), 4.92 (s, 2H), 7.64 (t, J=7.03 Hz, 1H), 7.75 (m, 1H), 7.80 (s, 1H), 7.88 (d, J=8.20 Hz, 1H), 8.08 (d, J=8.20 Hz, 1H), 9.21 (s, 1H). MS [M+H]⁺ 474.2 (ESI). HRMS m/z calcd for C₂₆H₃₂N₇O₂ [M+H]⁺ 474.2612, found 474.2607.

### Example 312

### 2-(4-acetylpiperazin-1-yl)-6-sec-butyl-4-(1-(4-fluorophenyl)-2-methylpropan-2-ylamino)-5H-pyrrolo[3,4-d]pyrimidin-7(6H)-one

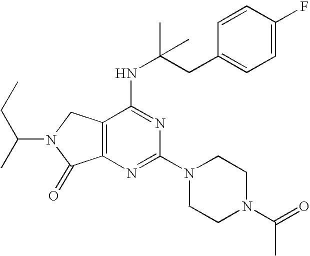

Following a procedure similar to that described in General Procedure 2, starting from 6-sec-butyl-2-chloro-4-(1-(4-fluorophenyl)-2-methylpropan-2-ylamino)-5H-pyrrolo[3,4-d]pyrimidin-7(6H)-one (Intermediate 87) and after purification by silica gel chromatography (4:4:0.4 hexane/ethyl acetate/methanol) followed by preparative LCMS (45-65% acetonitrile/water, pH=10), the title compound (35 mg, 19%) was obtained as a solid. HPLC: k′ 17.13; Purity: >98% (215 nm), >99% (254 nm), >99% (280 nm); Rₜ: 1.904 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 0.86 (t, J=7.42 Hz, 3H), 1.26 (d, J=6.64 Hz, 3H), 1.46 (d, J=2.34 Hz, 6H), 1.58-1.69 (m, 1H), 2.14 (s, 3H), 3.28-3.33 (m, 4H), 3.34 (s, 1H), 3.59-3.70 (m, 4H), 3.85-3.99 (m, 2H), 4.04 (q, J=13.7 Hz, 2H), 4.24-4.34 (m, 1H), 6.92 (t, J=8.79 Hz, 2H), 7.00-7.05 (m, 2H). M.S. (calcd): 483.59 (MH⁺), M.S. (found): 483.3 (ESI) (MH⁺).

### Example 313

### 2-(4-acetylpiperazin-1-yl)-4-(1-(4-fluorophenyl)-2-methylpropan-2-ylamino)-6-(3-methylbutan-2-yl)-5H-pyrrolo[3,4-d]pyrimidin-7(6H)-one

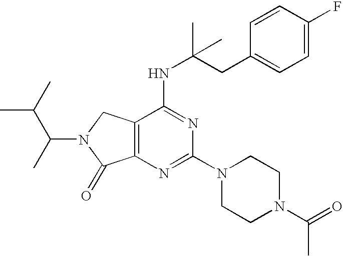

Following a procedure similar to that described in General Procedure 2, starting from 2-chloro-4-(1-(4-fluorophenyl)-2-methylpropan-2-ylamino)-6-(3-methylbutan-2-yl)-5H-pyrrolo[3,4-d]pyrimidin-7(6H)-one (Intermediate 84) and after purification by silica gel chromatography (4:4:0.4 hexane/ethyl acetate/methanol) followed by preparative LCMS (45-65% acetonitrile/water, pH=10), the title compound (35 mg, 33.6%) was obtained as a solid. HPLC: k′ 18.47; Purity: >99% (215 nm), >99% (254 nm), >99% (280 nm); Rₜ: 2.044 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 0.81 (d, J=6.64 Hz, 3H), 1.02 (d, J=6.64 Hz, 3H), 1.27 (d, J=7.03 Hz, 3H), 1.46 (s, 3H), 1.45 (s, 3H), 1.75-1.90 (m, 1H), 2.15 (s, 3H), 3.20-3.40 (m, 2H), 3.65-3.70 (m, 4H), 3.85-3.92 (m, 2H), 3.92-3.99 (m, 2H), 4.05 (d, J=17.97 Hz, 1H), 4.10 (d, J=17.97 Hz, 1H), 6.91 (t, J=8.79 Hz, 2H), 7.02 (dd, J=8.59, 5.47 Hz, 2H). M.S. (calcd): 497.62 (MH⁺), M.S. (found): 497.2 (ESI) (MH⁺).

### Example 314

### 2-(4-acetylpiperazin-1-yl)-4-(1-(4-fluorophenyl)-2-methylpropan-2-ylamino)-6-(1-methoxypropan-2-yl)-5H-pyrrolo[3,4-d]pyrimidin-7(6H)-one

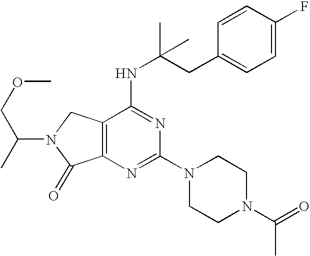

Following a procedure similar to that described in General Procedure 2, starting from 2-chloro-4-(1-(4-fluorophenyl)-2-methylpropan-2-ylamino)-6-(1-methoxypropan-2-yl)-5H-pyrrolo[3,4-d]pyrimidin-7(6H)-one (Intermediate 85) and after purification by silica gel chromatography (4:4:0.4 hexane/ethyl acetate/methanol) followed by preparative LCMS (45-65% acetonitrile/water, pH=10), the title compound (41 mg, 26.5%) was obtained as a solid. HPLC: k′ 15.58; Purity: >98% (215 nm), >99% (254 nm), >99% (280 nm); Rₜ: 1.741 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.26 (d, J=7.03 Hz, 3H), 1.45 (s, 3H), 1.46 (s, 3H), 2.14 (s, 3H), 3.20-3.40 (m, 2H), 3.49 (dd, J=10.2, 4.7 Hz, 1H), 3.55 (dd, J=10.2, 7.8 Hz, 1H), 3.60-3.71 (m, 4H), 3.85-4.00 (m, 4H), 4.12 (s, 2H), 4.50-4.62 (m, 1H), 6.97-6.97 (m, 2H), 6.97-7.06 (m, 2H). M.S. (calcd): 499.6 (MH⁺), M.S. (found): 499.2 (ESI) (MH⁺).

### Example 315

### 2-(4-acetylpiperazin-1-yl)-6-(2-chlorobenzyl)-4-(1-(4-fluorophenyl)-2-methylpropan-2-ylamino)-5H-pyrrolo[3,4-d]pyrimidin-7(6H)-one

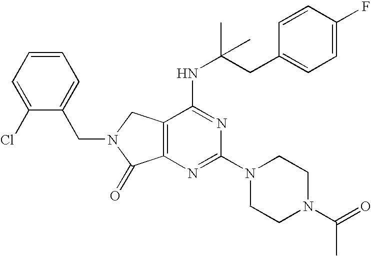

Following a procedure similar to that described in General Procedure 2, starting from 2-chloro-6-(2-chlorobenzyl)-4-(1-(4-fluorophenyl)-2-methylpropan-2-ylamino)-5H-pyrrolo[3,4-d]pyrimidin-7(6H)-one (Intermediate 86) and after purification by silica gel chromatography (4:4:0.4 hexane/ethyl acetate/methanol) followed by preparative LCMS (45-65% acetonitrile/water, pH=10), the title compound (38 mg, 34.3%) was obtained as a solid. HPLC: k′ 20.24; Purity: >98% (215 nm), >99% (254 nm), >99% (280 nm); Rₜ: 2.230 minutes; Conditions: Column: Zorbax SB C-18; Gradient: 05-95% B in 4.5 min, 70° C. Solvents: A: 0.05% TFA in H₂O, B: 0.05% TFA in MeCN, T₀=0.132 min. ¹H NMR (400 MHz, CD₃OD) δ ppm 1.41 (s, 6H), 2.15 (s, 3H), 3.25-3.28 (m, 2H), 3.60-3.70 (m, 4H), 3.85-3.98 (m, 4H), 3.99 (s, 2H), 4.90 (s, 2H), 6.85-6.92 (m, 2H), 6.98 (m, 2H), 6.94-7.03 (m, 2H), 7.28-7.34 (m, 2H), 7.41-7.46 (m, 1H). M.S. (calcd): 552.05 (MH⁺), M.S. (found): 551.2 (ESI) (MH⁺).

### Example 316

### 2-(4-acetylpiperazin-1-yl)-6-[(1S)-1-methylpropyl]-4-[(quinolin-3-ylmethyl)amino]-5,6-dihydro-7H-pyrrolo[3,4-d]pyrimidin-7-one

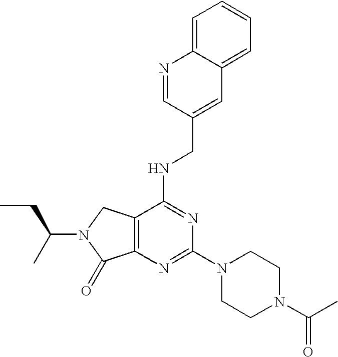

Following a procedure similar to that described in General Procedure 1, starting from (S)-6-sec-butyl-2,4-dichloro-5H-pyrrolo[3,4-d]pyrimidin-7(6H)-one (Intermediate 19) and after purification by preparative TLC (eluant 10% MeOH in EtOAc) followed by preparative LCMS (gradient 25-45% CH₃CN in H₂O containing 10 mM NH₄HCO₃) and lyophilization from CH₃CN/H₂O, the title compound was obtained as a solid (14.8 mg, 27.1%). HPLC purity: >98% (215 nm), >98% (254 nm), >97% (280 nm); Rₜ: 1.28 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} +17.2 (c=0.1, MeOH). ¹H NMR (400 MHz, CD₃OD) δ ppm 0.89 (t, J=7.23 Hz, 3H), 1.30 (d, J=7.03 Hz, 3H), 1.60-1.75 (m, 2H), 2.09 (s, 3H), 3.39-3.47 (m, 2H), 3.48-3.55 (m, 2H), 3.71-3.80 (m, 2H), 3.80-3.85 (m, 2H), 4.11-4.40 (m, 3H), 4.91 (s, 2H), 7.61 (t, J=7.03 Hz, 1H), 7.71-7.78 (m, 1H), 7.93 (d, J=8.20 Hz, 1H), 8.02 (d, J=8.98 Hz, 1H), 8.33 (s, 1H), 8.91 (d, J=1.95 Hz, 1H). M.S. 474.2 (ESI) (MH⁺)⁺. HRMS m/z calcd for C₂₆H₃₂N₇O₂ [M+H]⁺ 474.2610, found 474.2613.

### Example 317

### 2-(4-acetylpiperazin-1-yl)-4-[ethyl(isoquinolin-3-ylmethyl)amino]-6-(1-methylpropyl)-5,6-dihydro-7H-pyrrolo[3,4-d]pyrimidin-7-one (Enantiomer 1) and

### Example 318

### 2-(4-acetylpiperazin-1-yl)-4-[ethyl(isoquinolin-3-ylmethyl)amino]-6-(1-methylpropyl)-5,6-dihydro-7H-pyrrolo[3,4-d]pyrimidin-7-one (Enantiomer 2)

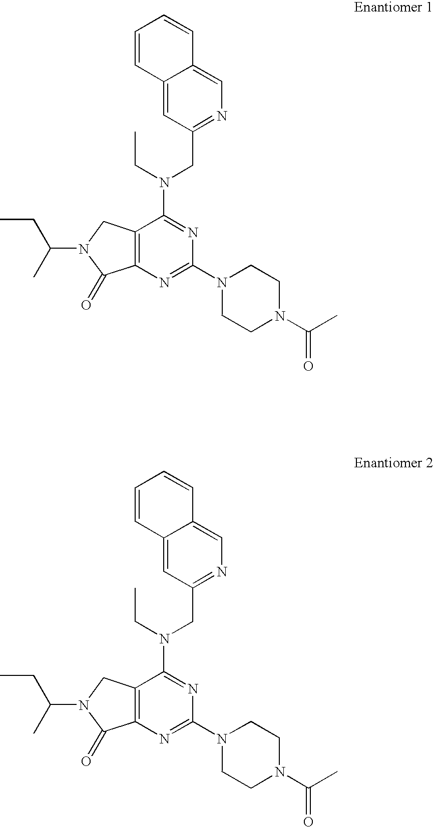

Following a procedure similar to that described in General Procedure 1, starting from 6-sec-butyl-2,4-dichloro-5H-pyrrolo[3,4-d]pyrimidin-7(6H)-one (Intermediate 18) and N-(isoquinoline-3-ylmethyl)ethanamine (Intermediate 62B) and after purification by silica gel chromatography (gradient 2-20% MeOH in EtOAc) followed by preparative LCMS (gradient 25-45% CH₃CN in H₂O containing 10 mM NH₄HCO₃) and lyophilization from CH₃CN/H₂O, a mixture of two enantiomers was obtained, which was then separated by SFC (AS column with EtOH+0.1% DMEA, Iso at 40%) to provide (+)-2-(4-acetylpiperazin-1-yl)-4-[ethyl(isoquinolin-3-ylmethyl)amino]-6-(1-methylpropyl)-5,6-dihydro-7H-pyrrolo[3,4-d]pyrimidin-7-one (65.9 mg, 8.95%) as a solid and (−)-2-(4-acetylpiperazin-1-yl)-4-[ethyl(isoquinolin-3-ylmethyl)amino]-6-(1-methylpropyl)-5,6-dihydro-7H-pyrrolo[3,4-d]pyrimidin-7-one (80.00 mg, 10.86%) as a solid following lyophilization from CH₃CN/H₂O.

Enantiomer 1: Purity: >95% (215 nm), >95% (254 nm), >91% (280 nm); Rₜ: 1.52 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} +17.5 (c=0.1, MeOH), 99% ee. ¹H NMR (400 MHz, CD₃OD) δ ppm 0.81 (t, J=7.23 Hz, 3H), 1.26 (d, J=7.03 Hz, 3H), 1.32 (t, J=7.03 Hz, 3H), 1.63 (m, 2H), 2.07 (s, 3H), 3.36-3.54 (m, 4H), 3.62-3.90 (m, 6H), 4.21-4.36 (m, 1H), 4.53 (s, 2H), 5.05 (s, 2H), 7.62-7.69 (m, 2H), 7.75 (t, J=7.03 Hz, 1H), 7.87 (d, J=8.20 Hz, 1H), 8.09 (d, J=8.20 Hz, 1H), 9.24 (s, 1H). M.S. 502.2 (ESI) (MH⁺)+. HRMS m/z calcd for C₂₈H₃₆N₇O₂ [M+H]⁺ 502.2925, found 502.2916.

Enantiomer 2: Purity: >98% (215 nm), >98% (254 nm), >97% (280 nm); Rₜ: 1.52 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D} −13.6 (c=0.1, MeOH), 91.1% ee. ¹H NMR (400 MHz, CD₃OD) δ ppm 0.73-0.87 (m, 3H), 1.25 (d, J=6.25 Hz, 3H), 1.32 (t, J=7.23 Hz, 3H), 1.55-1.73 (m, 2H), 2.07 (s, 3H), 3.36-3.53 (m, 4H), 3.63-3.90 (m, 6H), 4.21-4.36 (m, 1H), 4.38-4.62 (m, 2H), 5.05 (s, 2H), 7.61-7.69 (m, 2H), 7.75 (t, J=7.62 Hz, 1H), 7.86 (d, J=8.20 Hz, 1H), 8.09 (d, J=8.20 Hz, 1H), 9.24 (s, 1H). M.S. 502.2 (ESI) (MH⁺)+HRMS m/z calcd for C₂₈H₃₆N₇O₂ [M+H]⁺ 502.2925, found 502.2920.

### Example 319

### 2-(4-acetylpiperazin-1-yl)-6-[(1S)-1-methylpropyl]-4-[methyl(quinolin-3-ylmethyl)amino]-5,6-dihydro-7H-pyrrolo[3,4-d]pyrimidin-7-one

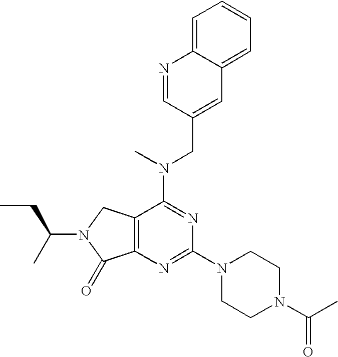

Following a procedure similar to that described in General Procedure 1, starting from (S)-6-sec-butyl-2,4-dichloro-5H-pyrrolo[3,4-d]pyrimidin-7(6H)-one (Intermediate 19) and after purification by silica gel chromatography (gradient 5-40% MeOH in EtOAc) followed by preparative LCMS (gradient 25-45% CH₃CN in H₂O containing 10 mM NH₄HCO₃) and lyophilization from CH₃CN/H₂O, the title compound was obtained as a solid (14.8 mg, 27.1%). HPLC purity: >98% (215 nm), >98% (254 nm), >97% (280 nm); Rₜ: 1.28 minutes; Conditions: Zorbax C-18, gradient 5-95% B in 4.5 min, flow rate 3.5 mL/min, 70° C., A: 0.05% TFA in H₂O, B: 0.05% TFA in CH₃CN. [α]_{D}=+17.2 (c=0.1, MeOH). ¹H NMR (400 MHz, CD₃OD) δ ppm 0.82-0.93 (m, 3H), 1.26-1.35 (m, 3H), 1.69 (dq, J=14.11, 7.15 Hz, 2H), 2.10 (s, 3H), 3.38 (s, 3H), 3.41-3.58 (m, 4H), 3.72-3.87 (m, 4H), 4.25-4.39 (m, 1H), 4.54-4.74 (m, 2H), 5.13 (s, 2H), 7.62 (t, J=7.03 Hz, 1H), 7.71-7.82 (m, 1H), 7.93 (d, J=8.20 Hz, 1H), 8.02 (d, J=8.59 Hz, 1H), 8.24 (s, 1H), 8.85 (d, J=1.95 Hz, 1H). M.S. 488.3 (ESI) (MH⁺)⁺.

### Example 320

### 2-(4-acetylpiperazin-1-yl)-4-{[(1R)-1-(4-ethoxy-3-fluorophenyl)ethyl]amino}-6-[(1S)-1-methylpropyl]-5,6-dihydro-7H-pyrrolo[3,4-d]pyrimidin-7-one

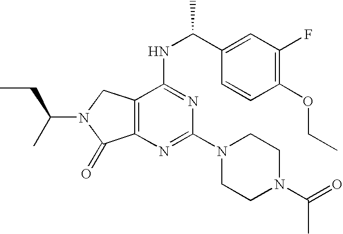

Following a procedure similar to that described in General Procedure 1, starting from (S)-6-sec-butyl-2,4-dichloro-5H-pyrrolo[3,4-d]pyrimidin-7(6H)-one (Intermediate 19) and (R)-1-(4-ethoxy-3-fluorophenyl)ethanamine hydrochloride (Intermediate 40), after purification by silica gel chromatography (10% MeOH in EtOAc) followed by preparative LCMS (gradient 25-45% CH₃CN in H₂O containing 10 mM NH₄HCO₃) and lyophilization from CH₃CN/H₂O, the title compound was obtained as a solid (135.0 mg, 74.1%). HPLC purity: >97% (215 nm), >97% (254 nm), >98% (280 nm); Rₜ: 1.78 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 0.89 (t, J=7.42 Hz, 3H), 1.30 (d, J=7.03 Hz, 3H), 1.38 (t, J=7.03 Hz, 3H), 1.53 (d, J=7.03 Hz, 3H), 1.63-1.73 (m, 2H), 2.12 (s, 3H), 3.40-3.60 (m, 4H), 3.70-3.89 (m, 4H), 4.07 (q, J=7.03 Hz, 2H), 4.20 (d, J=8.59 Hz, 2H), 4.25-4.35 (m, 1H), 5.16-5.24 (m, 1H), 6.97-7.03 (m, 1H), 7.07-7.14 (m, 2H). M.S. 499.2 (ESI) (MH⁺)⁺. HRMS m/z calcd for C₂₆H₃₆FN₆O₃ [M+H]⁺ 499.2827, found 499.2820.

### Example 321

### (R)-2-(4-acetylpiperazin-1-yl)-4-(1-(4-ethoxyphenyl)-2,2,2-trifluoroethylamino)-6-isopropyl-5H-pyrrolo[3,4-d]pyrimidin-7(6H)-one

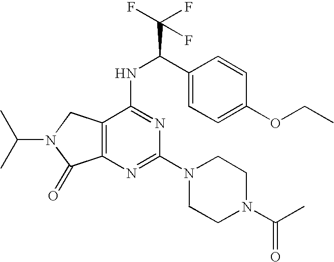

Following a procedure similar to that described in General Procedure 1, starting from (R)-1-(4-ethoxyphenyl)-2,2,2-trifluoroethanamine, after purification by preparative LCMS (gradient 50-70% CH₃CN in H₂O containing 10 mM NH₄HCO₃) and lyophilization from CH₃CN/H₂O, the title compound was obtained as a solid (50 mg, 36.5%). HPLC purity: >98% (215 nm), >99% (254 nm), >98% (280 nm); Rₜ: 2.05 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.21 (t, J=7.42 Hz, 6H), 1.31 (t, J=7.03 Hz, 3H), 2.04 (s, 3H), 3.45 (br. s., 4H), 3.57-3.85 (m, 4H), 4.02 (q, J=7.03 Hz, 2H), 4.22 (s, 2H), 4.37 (quin, J=6.74 Hz, 1H), 6.20 (quin, 1H), 6.95 (d, J=8.59 Hz, 2H), 7.55 (d, J=8.59 Hz, 2H), 8.43 (d, J=9.38 Hz, 1H). M.S. 521.3. (ESI) (MH⁺). HRMS m/z calcd for C₂₅H₃₂F₃N₆O₃[M+H]⁺ 521.24825, found 521.24868.

### Example 322

### (R)-2-(4-acetylpiperazin-1-yl)-4-(1-(4-(cyclopropylmethoxy)-3-fluorophenyl)ethylamino)-6-isopropyl-5H-pyrrolo[3,4-d]pyrimidin-7(6H)-one

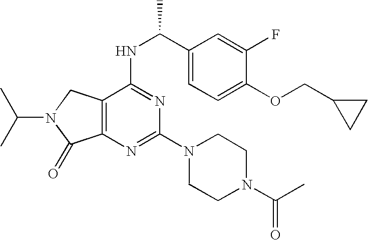

Following a procedure similar to that described in General Procedure 1, starting from (R)-1-(4-(cyclopropylmethoxy)-3-fluorophenyl)ethanamine hydrochloride (Intermediate 54), after purification by preparative LCMS (gradient 40-60% CH₃CN in H₂O containing 10 mM NH₄HCO₃) and lyophilization from CH₃CN/H₂O, the title compound was obtained as a solid (188 mg, 60.4%). [α]_{D}=+138.4 (c=0.01, MeOH). HPLC purity: >99% (215 nm), >99% (254 nm), >99% (280 nm); Rₜ: 1.84 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 0.22-0.36 (m, 2H), 0.48-0.63 (m, 2H), 1.20 (d, J=6.64 Hz, 7H), 1.44 (d, J=7.03 Hz, 3H), 2.02 (s, 3H), 3.24-3.49 (m, 4H), 3.50-3.77 (m, 4H), 3.83 (d, J=7.03 Hz, 2H), 4.15 (s, 2H), 4.36 (quin, J=6.64 Hz, 1H), 5.08-5.23 (m, 1H), 7.04 (t, J=8.40 Hz, 1H), 7.08-7.16 (m, 1H), 7.22 (dd, J=12.50, 1.95 Hz, 1H), 7.78 (d, J=7.03 Hz, 1H). M.S. 511.2. (ESI) (MH⁺). HRMS m/z calcd for C₂₇H₃₅FN₆O₃ [M+H]⁺ 511.28274, found 511.28296.

### Example 323

### 2-(4-acetylpiperazin-1-yl)-6-isopropyl-4-((2-methyl-2,3-dihydrobenzofuran-5-yl)methylamino)-5H-pyrrolo[3,4-d]pyrimidin-7(6H)-one

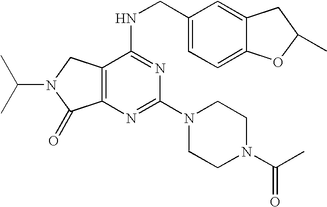

Following a procedure similar to that described in General Procedure 1, starting from (2-methyl-2,3-dihydrobenzofuran-5-yl)methanamine, after purification by preparative LCMS (gradient 40-60% CH₃CN in H₂O containing 10 mM NH₄HCO₃) and lyophilization from CH₃CN/H₂O, the title compound was obtained as a solid (222 mg, 78.0%). HPLC purity: >99% (215 nm), >99% (254 nm), >99% (280 nm); Rₜ: 1.49 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.18 (d, J=6.64 Hz, 6H), 1.34 (d, J=6.25 Hz, 3H), 2.03 (s, 3H), 2.73 (dd, J=15.62, 7.42 Hz, 1H), 3.26 (dd, J=15.62, 8.59 Hz, 1H), 3.44 (br. s., 4H), 3.59-3.83 (m, 4H), 4.10 (s, 2H), 4.35 (dt, J=13.57, 6.69 Hz, 1H), 4.47 (d, J=5.47 Hz, 2H), 4.76-4.98 (m, 1H), 6.65 (d, J=8.20 Hz, 1H), 7.07 (d, J=8.20 Hz, 1H), 7.18 (s, 1H), 7.91 (t, J=5.47 Hz, 1H). M.S. 465.2. (ESI) (MH⁺). HRMS m/z calcd for C₂₅H₃₂N₆O₃ [M+H]⁺ 465.26087, found 465.26076.

### Example 324

### 2-(4-acetylpiperazin-1-yl)-6-isopropyl-4-(1-(2-methyl-2,3-dihydrobenzofuran-5-yl)ethylamino)-5H-pyrrolo[3,4-d]pyrimidin-7(6H)-one

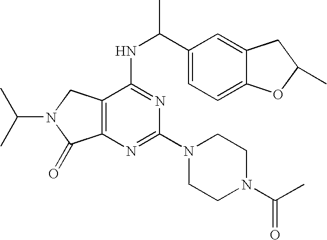

Following a procedure similar to that described in General Procedure 1, starting from 1-(2-methyl-2,3-dihydrobenzofuran-5-yl)ethanamine, after purification by preparative LCMS (gradient 30-50% CH₃CN in H₂O containing 10 mM NH₄HCO₃) and lyophilization from CH₃CN/H₂O, the title compound was obtained as a solid (168 mg, 57.5%). HPLC purity: >99% (215 nm), >99% (254 nm), >99% (280 nm); Rₜ: 1.60 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.19 (d, J=6.64 Hz, 6H), 1.34 (dd, J=6.25, 2.34 Hz, 3H), 1.44 (d, J=6.64 Hz, 3H), 2.02 (s, 3H), 2.73 (dd, J=15.62, 7.81 Hz, 0H), 3.26 (dd, J=15.62, 8.98 Hz, 1H), 3.31-3.51 (m, 4H), 3.53-3.82 (m, 4H), 4.13 (s, 2H), 4.36 (quin, J=6.74 Hz, 1H), 4.77-4.93 (m, 1H), 5.06-5.22 (m, 1H), 6.63 (d, J=8.20 Hz, 1H), 7.09 (d, J=8.20 Hz, 1H), 7.22 (s, 1H), 7.75 (d, J=7.42 Hz, 1H). M.S. 479.2. (ESI) (MH⁺). HRMS m/z calcd for C₂₆H₃₅N₆O₃ [M+H]⁺ 479.27652, found 479.27633.

### Example 325

### (S)—N-(2-(4-acetyl-3-methylpiperazin-1-yl)-6-isopropyl-7-oxo-6,7-dihydro-5H-pyrrolo[3,4-d]pyrimidin-4-yl)-N-(isoquinolin-3-ylmethyl)acetamide

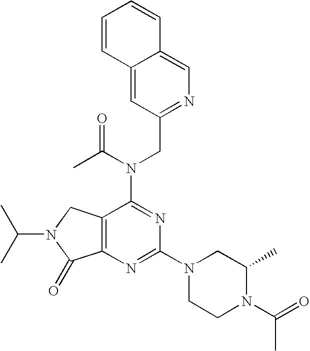

A solution of (S)-tert-butyl 4-(6-isopropyl-4-(isoquinolin-3-ylmethylamino)-7-oxo-6,7-dihydro-5H-pyrrolo[3,4-d]pyrimidin-2-yl)-2-methylpiperazine-1-carboxylate (Intermediate 120) (130 mg, 0.24 mmol) and 2,2,2-trifluoroacetic acid (0.908 ml, 12.23 mmol) in anhydrous DCM (5 ml) was stirred for 16 h at rt. After concentration under reduced pressure, the residue was dissolved in anhydrous DCM (3 ml), cooled to 0° C. and DIPEA (0.170 ml, 0.98 mmol) was added dropwise followed by acetyl chloride (0.035 ml, 0.49 mmol). The reaction mixture was allowed to warm up to rt and was stirred for 16 h. After concentration under reduced pressure, the residue was purified by preparative HPLC (gradient 35-55% CH₃CN in H₂O containing 10 mM NH₄HCO₃) to give the title compound (25.0 mg, 19.8%) as a solid. HPLC purity: >96% (215 nm), >97% (254 nm), >96% (280 nm); Rₜ: 1.572 minutes; Conditions: Zorbax SB C-18; Gradient: 05-95% B in 4.5 min, 70° C. A: 0.05% TFA in H₂O, B: 0.05% TFA in CH₃CN. ¹H NMR (DMSO-d₆) δ ppm 0.89 (d, J=6.25 Hz, 3H), 1.21 (d, J=6.64 Hz, 6H), 1.96 (s, 3H), 2.34 (s, 3H), 2.95 (br s, 1H), 3.05 (s, 3H), 3.11 (dd, J=13.48, 4.49 Hz, 1H), 4.16 (d, J=14.06 Hz, 1H), 4.22 (s, 2H), 4.28 (d, J=12.89 Hz, 1H), 4.35-4.44 (m, 1H), 5.31 (d, J=2.34 Hz, 2H), 7.60-7.66 (m, 1H), 7.72-7.78 (m, 2H), 7.90 (d, J=8.20 Hz, 1H), 8.08 (d, J=8.20 Hz, 1H), 9.23 (s, 1H). M.S. 516.3 (ESI) (MH⁺). HRMS m/z calcd for C₂₈H₃₄N₇O₃ [M+H]⁺ 516.2718, found 516.2721.

### Example 326

### (R)—N-(2-(4-acetyl-3-methylpiperazin-1-yl)-6-isopropyl-7-oxo-6,7-dihydro-5H-pyrrolo[3,4-d]pyrimidin-4-yl)-N-(isoquinolin-3-ylmethyl)acetamide

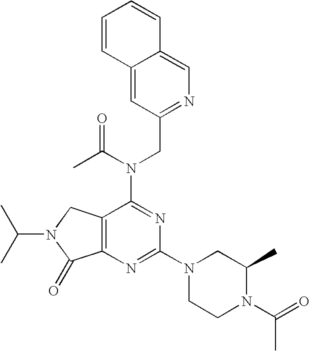

Following a procedure similar to that described for Example 325, starting from (R)-tert-butyl 4-(6-isopropyl-4-(isoquinolin-3-ylmethylamino)-7-oxo-6,7-dihydro-5H-pyrrolo[3,4-d]pyrimidin-2-yl)-2-methylpiperazine-1-carboxylate (Intermediate 121) and after purification by preparative HPLC (gradient 35-55% CH₃CN in H₂O containing 10 mM NH₄HCO₃), the title compound was obtained as a solid (31.0 mg, 22.8%). HPLC purity: >97% (215 nm), >97% (254 nm), >98% (280 nm); Rₜ: 1.608 minutes; Conditions: Zorbax SB C-18; Gradient: 05-95% B in 4.5 min, 70° C. A: 0.05% TFA in H₂O, B: 0.05% TFA in CH₃CN. ¹H NMR (DMSO-d₆) δ ppm 0.89 (d, J=6.64 Hz, 3H), 1.21 (d, J=6.64 Hz, 6H), 1.96 (s, 3H), 2.33 (s, 3H), 2.95 (bras, I H), 3.05 (s, 3H), 3.11 (dd, J=13.67, 4.30 Hz, 1H), 4.16 (d, J=14.06 Hz, 1H), 4.22 (s, 2H), 4.28 (d, J=12.50 Hz, 1H), 4.35-4.44 (m, 1H), 5.26-5.37 (m, 2H), 7.61-7.66 (m, 1H), 7.72-7.78 (m, 2H), 7.90 (d, J=8.20 Hz, 1H), 8.08 (dd, J=8.20, 0.78 Hz, 1H), 9.23 (s, 1H). M.S. 516.3 (ESI) (MH⁺) HRMS m/z calcd for C₂₈H₃₄N₇O₃ [M+H]⁺ 516.2718, found 516.2714.

### Example 327

### (S)-2-(4-acetyl-3-methylpiperazin-1-yl)-6-isopropyl-4-(quinolin-3-ylmethylamino)-5H-pyrrolo[3,4-d]pyrimidin-7(6H)-one

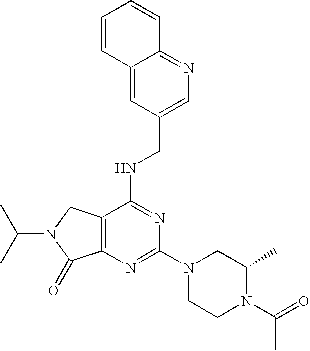

Following a procedure similar to that described for Example 325, starting from (s)-tert-butyl 4-(6-isopropyl-7-oxo-4-(quinolin-3-ylmethylamino)-6,7-dihydro-5H-pyrrolo[3,4-d]pyrimidin-2-yl)-2-methylpiperazine-1-carboxylate (Intermediate 122) and after purification by preparative HPLC (gradient 35-55% CH₃CN in H₂O containing 10 mM NH₄HCO₃), the title compound was obtained as a solid (64.0 mg, 47.9%). HPLC purity: >99% (215 nm), >99% (254 nm), >99% (280 nm); Rₜ: 1.088 minutes; Conditions: Zorbax SB C-18; Gradient: 05-95% B in 4.5 min, 70° C. A: 0.05% TFA in H₂O, B: 0.05% TFA in CH₃CN. ¹H NMR (DMSO-d₆) δ ppm 0.83 (d, J=58.98 Hz, 3H), 1.19 (d, J=6.64 Hz, 6H), 1.97 (d, J=12.11 Hz, 3H), 2.64-2.80 (m, 1H), 2.86-3.08 (m, 2H), 3.16 (t, J=12.70 Hz, 1H), 4.15 (s, 2H), 4.30-4.60 (m, 4H), 4.68-4.87 (m, 2H), 7.55-7.61 (m, 1H), 7.67-7.73 (m, 1H), 7.94 (dd, J=8.40, 0.98 Hz, 1H), 7.99 (d, J=8.20 Hz, 1H), 8.17 (t, J=5.86 Hz, 1H), 8.25 (d, J=1.17 Hz, 1H), 8.94 (d, J=1.95 Hz, 1H). M.S. 474.2 (ESI) (MH⁺). HRMS m/z calcd for C₂₆H₃₂N₇O₂ [M+H]⁺ 474.2612, found 474.2616.

### Example 328

### (R)-2-(4-acetyl-3-methylpiperazin-1-yl)-6-isopropyl-4-(quinolin-3-ylmethylamino)-5H-pyrrolo[3,4-d]pyrimidin-7(6H)-one

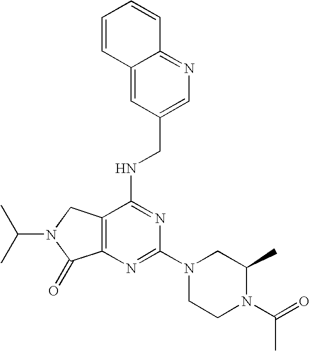

Following a procedure similar to that described for Example 325, starting from (R)-tert-butyl 4-(6-isopropyl-7-oxo-4-(quinolin-3-ylmethylamino)-6,7-dihydro-5H-pyrrolo[3,4-d]pyrimidin-2-yl)-2-methylpiperazine-1-carboxylate (Intermediate 123) and after purification by preparative HPLC (gradient 25-45% CH₃CN in H₂O containing 10 mM NH₄HCO₃), the title compound was obtained as a solid (62.0 mg, 46.4%). HPLC purity: >99% (215 nm), >99% (254 nm), >99% (280 nm); Rₜ: 1.100 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.04 (dd, J=36.52, 5.66 Hz, 3H), 1.22 (d, J=7.03 Hz, 6H), 2.07 (d, J=12.89 Hz, 3H), 2.87-3.02 (m, 2H), 3.11 (t, J=12.89 Hz, 1H), 3.26-3.55 (m, 1H), 4.09-4.14 (m, 2H), 4.32-4.80 (m, 4H), 4.81-4.96 (m, 2H), 5.54 (t, J=5.27 Hz, 1H), 7.54 (t, J=7.42 Hz, 1H), 7.67-7.73 (m, 1H), 7.75 (d, J=8.20 Hz, 1H), 8.05-8.10 (m, 1H), 8.91 (d, J=1.56 Hz, 1H). M.S. 474.2 (ESI) (MH⁺). HRMS m/z calcd for C₂₆H₃₂N₇O₂ [M+H]⁺ 474.2612, found 474.2609.

### Example 329

### 6-(1-methylethyl)-2-[4-(methylsulfonyl)piperazin-1-yl]-4-[(quinolin-3-ylmethyl)amino]-5,6-dihydro-7H-pyrrolo[3,4-d]pyrimidin-7-one

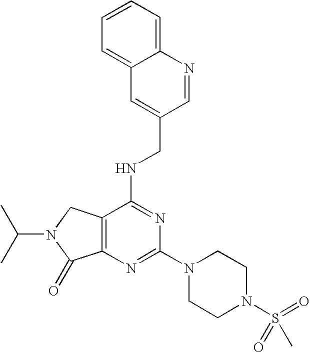

To a solution of 6-isopropyl-2-(piperazin-1-yl)-4-(quinolin-3-ylmethylamino)-5H-pyrrolo[3,4-d]pyrimidin-7(6H)-one (Intermediate 125) (0.159 g, 0.38 mmol) in dry DCM (4 mL) was added methanesulfonyl chloride (44 mg, 0.38 mmol) followed by TEA (0.053 mL, 0.38 mmol). The reaction mixture was stirred at rt for 2 h, concentrated under reduced pressure and the residue was purified by preparative LCMS (high pH) to give the title compound (95 mg, 50.4%) as a solid. ¹H NMR (400 MHz, CD₃OD) δ ppm 1.27 (d, J=7.03 Hz, 6H), 2.65 (s, 3H), 2.94-3.02 (m, 4H), 3.78-3.86 (m, 4H), 4.23 (s, 2H), 4.45-4.54 (m, 1H), 4.86-4.94 (m, 2H), 7.57 (t, J=7.42 Hz, 1H), 7.71 (t, J=7.23 Hz, 1H), 7.88 (d, J=7.42 Hz, 1H), 7.98 (d, J=8.20 Hz, 1H), 8.28 (s, 1H), 8.87 (d, J=1.95 Hz, 1H). M.S. 496.3 (ESI) (MH⁺); HRMS m/z calcd for C₂₄H₃₀N₇O₃S [M+H]⁺ 496.21253, found 496.21176.

### Example 330

### (R)-2-(4-acetylpiperazin-1-yl)-4-(1-(6-ethoxy-5-fluoropyridin-3-yl)ethylamino)-6-isopropyl-5H-pyrrolo[3,4-d]pyrimidin-7(6H)-one

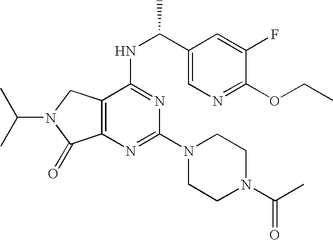

Following a procedure similar to that described in General Procedure 1, starting from (R)-1-(6-ethoxy-5-fluoropyridin-3-yl)ethanamine (Intermediate 126) and after purification by preparative LCMS (high pH) and lyophilization from CH₃CN/H₂O, the title compound (122 mg, 41.2% over 2 steps) was obtained as a solid. [α]_{D}=+137.1 (c=0.01, MeOH). HPLC purity: >96% (215 nm), >95% (254 nm), >93% (280 nm); Rₜ: 1.59 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) [symbol] ppm 1.20 (dd, J=6.64, 1.56 Hz, 6H), 1.31 (t, J=7.03 Hz, 3H), 1.48 (d, J=7.03 Hz, 3H), 2.02 (s, 3H), 3.22-3.51 (m, 4H), 3.54-3.83 (m, 4H), 4.15 (d, J=2.34 Hz, 2H), 4.28-4.45 (m, 3H), 5.22 (qd, J=6.90, 6.64 Hz, 1H), 7.68 (dd, J=11.52, 1.76 Hz, 1H), 7.81 (d, J=7.03 Hz, 1H), 8.01 (d, J=1.56 Hz, 1H). M.S. 486.2. (ESI) (MH⁺). HRMS m/z calcd for C₂₄H₃₄FN₇O₃ [M+H]⁺ 486.26234, found 486.26208.

### Example 331

### (R)-2-(4-acetylpiperazin-1-yl)-4-(1-(6-ethoxy-5-methylpyridin-3-yl)ethylamino)-6-isopropyl-5H-pyrrolo[3,4-d]pyrimidin-7(6H)-one

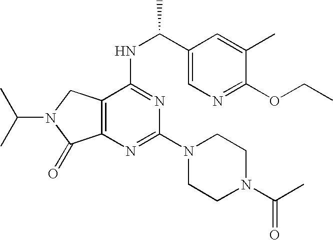

Following a procedure similar to that described in General Procedure 1, starting from (R)-1-(6-ethoxy-5-methylpyridin-3-yl)ethanamine (Intermediate 127) and after purification by preparative LCMS (high pH) and lyophilization from CH₃CN/H₂O, the title compound (32.7 mg, 26.1% over 2 steps) was obtained as a solid. [α]_{D}=+115.2 (c=0.01, MeOH) HPLC purity: >97% (215 nm), >97% (254 nm), >98% (280 nm); Rₜ: 1.64 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.19 (d, J=5.47 Hz, 6H), 1.29 (t, J=7.03 Hz, 3H), 1.47 (d, J=6.64 Hz, 3H), 2.02 (s, 3H), 2.11 (s, 4H), 3.28-3.49 (m, 4H), 3.57-3.77 (m, 4H), 4.13 (s, 2H), 4.28 (q, J=7.03 Hz, 2H), 4.36 (quin, J=6.74 Hz, 1H), 5.11-5.21 (m, 1H), 7.55 (s, 1H), 7.79 (d, J=7.42 Hz, 1H), 7.99 (s, 1H). M.S. 482.2. (ESI) (MH⁺). HRMS m/z calcd for C₂₅H₃₆N₇O₃ [M+H]⁺ 482.28741, found 482.28766.

### Example 332

### (R)-2-(4-acetylpiperazin-1-yl)-4-(1-(4-(hydroxymethyl)phenyl)ethylamino)-6-isopropyl-5H-pyrrolo[3,4-d]pyrimidin-7(6H)-one

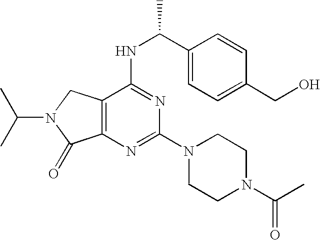

Following a procedure similar to that described in General Procedure 1, starting from (R)-(4-(1-aminoethyl)phenyl)methanol (Intermediate 129) and after purification by preparative LCMS (high pH) and lyophilization from CH₃CN/H₂O, the title compound (94.3 mg, 33.8% over 2 steps) was obtained as a solid. [α]_{D}=+125.0 (c=0.01, MeOH). HPLC purity: >96% (215 nm), >97% (254 nm), >98% (280 nm); Rₜ: 1.14 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.64 Hz, 6H), 1.46 (d, J=7.03 Hz, 3H), 2.02 (s, 3H), 3.20-3.46 (m, 4H), 3.49-3.76 (m, 4H), 4.15 (s, 2H), 4.36 (dt, J 13.28, 6.64 Hz, 1H), 4.44 (d, J=5.08 Hz, 2H), 5.09 (t, J=5.66 Hz, 1H), 5.19 (qd, J'6.90, 6.64 Hz, 1H), 7.24 (d, J=8.20 Hz, 2H), 7.34 (d, J=8.20 Hz, 2H), 7.83 (d, J=7.03 Hz, 1H). M.S. 453.3 (ESI) (MH⁺). HRMS m/z calcd for C₂₄H₃₃N₆O₃ [M+H]⁺ 453.26087, found 453.26105.

### Example 333

### 2-(4-acetylpiperazin-1-yl)-6-isopropyl-4-(2-methyl-1-m-tolylpropan-2-ylamino)-5H-pyrrolo[3,4-d]pyrimidin-7(6H)-one

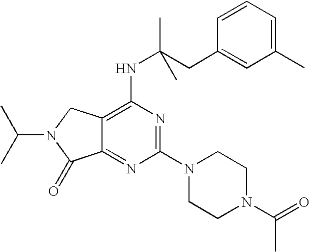

Following a procedure similar to that described in General Procedure 2, starting from 2-chloro-6-isopropyl-4-(2-methyl-1-m-tolylpropan-2-ylamino)-5H-pyrrolo[3,4-d]pyrimidin-7(6H)-one (Intermediate 131) and after purification by preparative LCMS (gradient 55-75% CH₃CN in H₂O containing 10 mM NH₄HCO₃) and lyophilization from CH₃CN/H₂O, the title compound was obtained as a solid (34.0 mg, 13.64%). Purity: >99% (215 nm), >99% (254 nm), >99% (280 nm); Rₜ: 1.93 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.24 (d, J=6.64 Hz, 6H), 1.49 (s, 6H), 2.15 (s, 3H), 2.27 (s, 3H), 3.16 (s, 2H), 3.54 (dd, J=6.05, 4.49 Hz, 2H), 3.68-3.74 (m, 2H), 3.87-3.93 (m, 4H), 3.95-4.00 (m, 2H), 4.11 (s, 1H), 4.62-4.72 (m, 1H), 6.82 (dd, J=3.32, 0.98 Hz, 2H), 7.01-7.07 (m, 1H), 7.11-7.17 (m, 1H). MS [M+H]⁺ 465.2 (ESI). HRMS m/z calcd for C₂₆H₃₇N₆O₂ [M+H]⁺ 465.29725, found 465.29765.

### Example 334

### 2-(4-acetylpiperazin-1-yl)-4-((4-ethoxy-2-methoxybenzyl)(methyl)amino)-6-isopropyl-5H-pyrrolo[3,4-d]pyrimidin-7(6H)-one

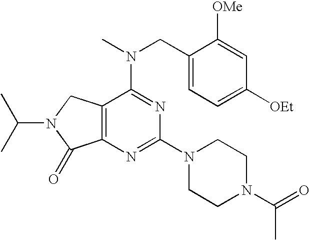

Following a procedure similar to that described in General Procedure 1, starting from 1-(4-ethoxy-2-methoxyphenyl)-N-methylmethanamine (Intermediate 133) and after purification by preparative LCMS (gradient 45-65% CH₃CN in H₂O containing 10 mM NH₄HCO₃) and lyophilization from CH₃CN/H₂O, the title compound was obtained as a solid (75 mg, 42.2%). Purity: >99% (215 nm), >99% (254 nm), >99% (280 nm); Rₜ: 1.79 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.21 (d, J=6.64 Hz, 6H), 1.42 (t, J=7.03 Hz, 3H), 2.14 (s, 3H), 3.18 (s, 3H), 3.46-3.53 (m, 2H), 3.63-3.69 (m, 2H), 3.81 (s, 3H), 3.82-3.87 (m, 2H), 3.89-3.96 (m, 2H), 4.02 (q, J=7.03 Hz, 2H), 4.29 (s, 2H), 4.58-4.70 (m, 3H), 6.42 (dd, J=8.20, 2.34 Hz, 1H), 6.50 (d, J=2.34 Hz, 1H), 6.95 (d, 1H). M.S. 497.2 (ESI) (MH⁺). HRMS m/z calcd for C₂₆H₃₇N₆O₄[M+H]⁺ 497.28708, found 497.28701

### Example 335

### 2-(4-acetylpiperazin-1-yl)-4-(4-ethoxy-2-methoxybenzylamino)-6-isopropyl-5H-pyrrolo[3,4-d]pyrimidin-7(6H)-one

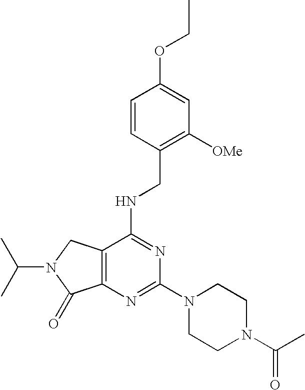

Following a procedure similar to that described in General Procedure 1, starting from (4-ethoxy-2-methoxyphenyl)methanamine (Intermediate 134) and after purification by preparative LCMS (gradient 45-65% CH₃CN in H₂O containing 10 mM NH₄HCO₃) and lyophilization from CH₃CN/H₂O, the title compound was obtained as a solid (31 mg, 27.9%). Purity: >98% (215 nm), >99% (254 nm), >99% (280 nm); Rₜ: 1.62 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm 1.25 (d, J=6.64 Hz, 6H), 1.42 (t, J=6.84 Hz, 3H), 2.16 (s, 3H), 3.48-3.54 (m, 2H), 3.66-3.73 (m, 2H), 3.86 (s, 3H), 3.88-3.93 (m, 2H), 3.96 (d, J=5.08 Hz, 2H), 4.00-4.08 (m, 4H), 4.58-4.71 (m, 3H), 5.04 (br. s., 1H), 6.44 (dd, J=8.20, 2.34 Hz, 1H), 6.50 (d, J=2.34 Hz, 1H), 7.20 (d, 1H). MS [M+H]⁺ 483.3 (ESI). HRMS m/z calcd for C₂₅H₃₅N₆O₄ [M+H]⁺ 483.27143, found 483.27146.

### Example 336

### 2-(4-acetylpiperazin-1-yl)-4-((2-ethoxy-4-methoxybenzyl)(methyl)amino)-6-isopropyl-5H-pyrrolo[3,4-d]pyrimidin-7(6H)-one

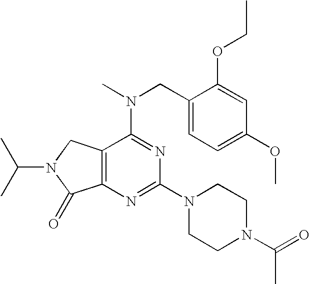

Following a procedure similar to that described in General Procedure 1, starting from 1-(2-ethoxy-4-methoxyphenyl)-N-methylmethanamine (Intermediate 136) and the intermediate 2-chloro-4-((4-ethoxy-2-methoxybenzyl)(methyl)amino)-6-isopropyl-5H-pyrrolo[3,4-d]pyrimidin-7(6H)-one (0.145 g, 58.8%) was isolated after silica gel chromatography (0-10% MeOH/DCM) M.S. [M+H]⁺ 405.1 (ESI). It was then converted to the title compound as a solid (17 mg, 13.9%), following purification by preparative LCMS (gradient 45-65% CH₃CN in H₂O containing 10 mM NH₄HCO₃) and lyophilization from CH₃CN/H₂O₀ Purity: >98% (215 nm), >98% (254 nm), >99% (280 nm); Rₜ: 1.75 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD) δ ppm 1.22 (d, J=6.64 Hz, 6H), 1.33 (t, J=7.03 Hz, 3H), 2.14 (s, 3H), 3.15 (s, 3H), 3.46-3.53 (m, 2H), 3.63-3.69 (m, 2H), 3.80 (s, 3H), 3.82-3.88 (m, 2H), 3.90-3.95 (m, 2H), 4.02 (q, J=7.03 Hz, 2H), 4.32 (s, 2H), 4.60-4.72 (m, 3H), 6.43 (dd, J=8.20, 2.34 Hz, 1H), 6.47 (d, J=2.34 Hz, 1H), 7.00 (d, 1H). MS [M+H]⁺ 497.2 (ESI). HRMS m/z calcd for C₂₆H₃₇N₆O₄[M+H]⁺ 497.28708, found 497.28682.

### Example 337

### 2-(4-acetylpiperazin-1-yl)-4-(4-isopropoxy-2-methoxybenzylamino)-6-isopropyl-5H-pyrrolo[3,4-d]pyrimidin-7(6H)-one

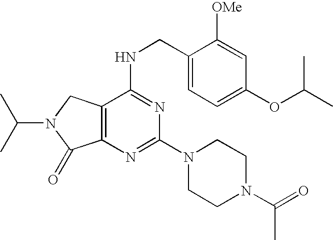

Following a procedure similar to that described in General Procedure 1, starting from (4-isopropoxy-2-methoxyphenyl)methanamine (Intermediate 137) and the intermediate 2-chloro-4-(4-isopropoxy-2-methoxybenzylamino)-6-isopropyl-5H-pyrrolo[3,4-d]pyrimidin-7(6H)-one (0.128 g, 78%) was isolated after silica gel chromatography (0-10% MeOH/DCM) M.S.

[M+H]⁺ 405.07 (ESI). It was then converted to the title compound as a solid (43 mg, 35%), following purification by preparative LCMS (gradient 45-65% CH₃CN in H₂O containing 10 mM NH₄HCO₃) and lyophilization from CH₃CN/H₂O. Purity: >99% (215 nm), >99% (254 nm), >99% (280 nm); Rₜ: 1.76 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD), δ ppm 1.24 (d, J=6.64 Hz, 6H), 1.34 (d, J=6.25 Hz, 6H), 2.15 (s, 3H), 3.48-3.53 (m, 2H), 3.65-3.72 (m, 2H), 3.85 (s, 3H), 3.88-3.93 (m, 2H), 3.93-3.99 (m, 2H), 4.03 (s, 2H), 4.55 (quin, J=5.96 Hz, 1H), 4.60 (d, J=5.86 Hz, 2H), 4.66 (quin, J=6.71, 6.45 Hz, 1H), 4.89 (br. s., 1H), 6.44 (dd, J=8.20, 2.34 Hz, 1H), 6.47 (d, J=2.34 Hz, 1H), 7.19 (d, 1H). MS [M+H]⁺ 497.2 (ESI). HRMS m/z calcd for C₂₆H₃₇N₆O₄[M+H]⁺ 497.28708, found 497.28690.

### Example 338

### 2-(4-acetylpiperazin-1-yl)-4-({(1R)-1-[3-fluoro-4-(methoxymethyl)phenyl]ethyl}amino)-6-(1-methylethyl)-5,6-dihydro-7H-pyrrolo[3,4-d]pyrimidin-7-one

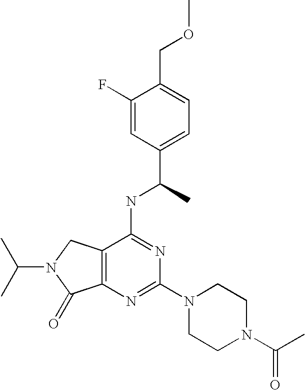

Following a procedure similar to that described in General Procedure 1, starting from (R)-1-(3-fluoro-4-(methoxymethyl)phenyl)ethanamine (Intermediate 139) and after purification by silica gel chromatography (gradient 5-40% MeOH in EtOAc) followed by preparative LCMS (gradient 30-50% CH₃CN in H₂O containing 10 mM NH₄HCO₃) and lyophilization from CH₃CN/H₂O, the title compound was obtained as a solid (111.0 mg, 44.0%). HPLC purity: >95% (215 nm), >93% (254 nm), >95% (280 nm); Rₜ: 1.55 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.31 (d, J=6.64 Hz, 6H), 1.54 (d, J=7.03 Hz, 3H), 2.10 (s, 3H), 3.33-3.54 (m, 7H), 3.57-3.88 (m, 4H), 4.26 (d, J=2.73 Hz, 2H), 4.46 (s, 2H), 4.52 (quin, J=6.74 Hz, 1H), 5.15-5.28 (m, 1H), 7.10 (dd, J=10.94, 1.56 Hz, 1H), 7.19 (dd, J=7.81, 1.56 Hz, 1H), 7.35 (t, J=7.62 Hz, 1H). M.S. 485.2 (ESI) (MH⁺)⁺. HRMS m/z calcd for C₂₅H₃₄FN₆O₃ [M+H]⁺ 485.2670, found 485.2670.

### Example 339

### 2-(4-acetylpiperazin-1-yl)-4-[{[1-(4-fluorophenyl)-1H-pyrazol-4-yl]methyl}(methyl)amino]-6-(1-methylethyl)-5,6-dihydro-7H-pyrrolo[3,4-d]pyrimidin-7-one

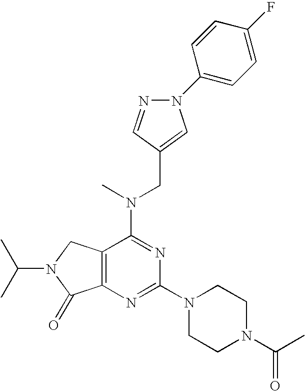

Following a procedure similar to that described in General Procedure 1 and after purification by silica gel chromatography (gradient 5-40% MeOH in EtOAc) then preparative LCMS (gradient 25-45% CH₃CN in H₂O containing 10 mM NH₄HCO₃) and lyophilization from CH₃CN/H₂O, the title compound was obtained as a solid (163.0 mg, 62.8%). HPLC purity: >98% (215 nm), >97% (254 nm), >97% (280 nm); Rₜ: 1.58 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=7.03 Hz, 6H), 2.10 (s, 3H), 3.25 (s, 3H), 3.53-3.57 (m, 2H), 3.58-3.62 (m, 2H), 3.80-3.85 (m, 2H), 3.86-3.91 (m, 2H), 4.46-4.55 (m, 1H), 4.61 (s, 2H), 4.78 (s, 2H), 7.14-7.22 (m, 2H), 7.63-7.71 (m, 3H), 8.13 (s, 1H). M.S. 507.2 (ESI). HRMS m/z calcd for C₂₆H₃₂FN₈O₂ [M+H]⁺ 507.2626, found 507.2622.

### Example 340

### 2-(5,6-dihydroimidazo[1,2-a]pyrazin-7(8H)-yl)-4-{[2-(4-fluorophenyl)-1,1-dimethylethyl]amino}-6-(1-methylethyl)-5,6-dihydro-7H-pyrrolo[3,4-d]pyrimidin-7-one

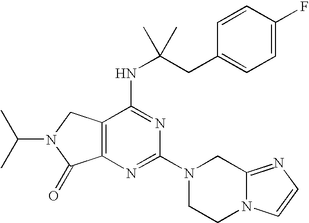

Following a procedure similar to that described in General Procedure 1 and after purification by silica gel chromatography (gradient 5-40% MeOH in EtOAc) then preparative LCMS (gradient 40-60% CH₃CN in H₂O containing 10 mM NH₄HCO₃) and lyophilization from CH₃CN/H₂O, the title compound was obtained as a solid (20.0 mg, 8.85%). HPLC purity: >98% (215 nm), >98% (254 nm), >98% (280 nm); Rₜ: 1.67 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.29 (d, J=6.64 Hz, 6H), 1.49 (s, 6H), 3.35-3.37 (m, 2H), 4.11 (s, 2H), 4.21 (t, J=5.47 Hz, 2H), 4.31 (t, J=5.27 Hz, 2H), 4.49-4.57 (m, 1H), 5.09 (s, 2H), 6.84 (s, 1H), 6.89-6.97 (m, 2H), 7.01-7.08 (m, 2H), 7.62 (s, 1H). M.S. 464.2 (ESI). HRMS m/z calcd for C₂₅H₃₁FN₇O [M+H]⁺ 464.2568, found 464.2571.

### Example 341

### 2-(4-acetylpiperazin-1-yl)-4-(((7-chloroisoquinolin-3-yl)methyl)(methyl)amino)-6-isopropyl-5H-pyrrolo[3,4-d]pyrimidin-7(6H)-one

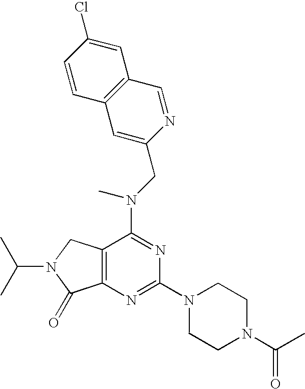

Following a procedure similar to that described in General Procedure 1, starting from 1-(7-chloroisoquinolin-3-yl)-N-methylmethanamine (Intermediate 140) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (13.50 mg, 18.31%). HPLC purity: >96% (215 nm), >95% (254 nm), >95% (280 nm); Rₜ: 1.31 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (d, J=7.0 Hz, 6H), 1.97 (s, 3H), 3.32 (s, 3H), 3.25-3.42 (m, 4H), 3.50-3.68 (m, 4H), 4.30-4.45 (m, 1H), 4.61 (s, 2H), 5.02 (s, 2H), 7.75 (s, 1H), 7.77 (dd, J=9.0, 2.4 Hz, 1H), 8.00 (d, J=9.0 Hz, 1H), 8.25 (d, J=2.4 Hz, 1H), 9.29 (s, 1H). M.S. 508.3. (ESI) (MH⁺). HRMS m/z calcd for C₂₆H₃₁ClN₇O₂ [M+H]⁺ 508.2150, found 508.2221.

### Example 342

### 2-(4-acetylpiperazin-1-yl)-6-isopropyl-4-(methyl((6-methylisoquinolin-3-yl)methyl)amino)-5H-pyrrolo[3,4-d]pyrimidin-7(6H)-one

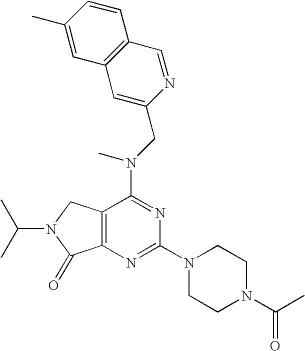

Following a procedure similar to that described in General Procedure 1, starting from N-methyl-1-(6-methylisoquinolin-3-yl)methanamine (Intermediate 141) and after purification by preparative LCMS (gradient 25-45% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (45.4 mg, 40.4%). HPLC purity: >99% (215 nm), >99% (254 nm), >99% (280 nm); Rₜ: 1.31 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (d, J=7.0 Hz, 6H), 1.98 (s, 3H), 2.48 (s, 3H), 3.30-3.44 (m, 4H), 3.52-3.72 (m, 7H), 4.30-4.45 (m, 1H), 4.60 (s, 2H), 5.00 (s, 2H), 7.47 (d, J=8.4 Hz, 1H), 7.58 (s, 1H), 7.69 (s, 1H), 7.99 (d, J=8.4 Hz, 1H), 9.21 (s, 1H). M.S. 488.3. (ESI) (MH⁺). HRMS m/z calcd for C₂₇H₃₄N₇O₂ [M+H]⁺ 488.2696, found 488.2768.

### Example 343

### 2-(4-acetylpiperazin-1-yl)-6-isopropyl-4-(quinolin-3-ylmethylamino)-5H-pyrrolo[3,4-d]pyrimidin-7(6H)-one

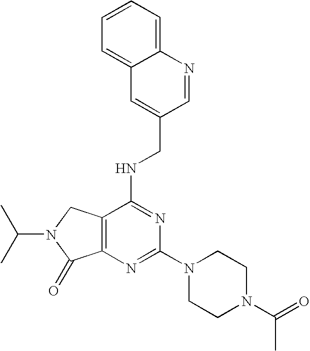

Following a procedure similar to that described in General Procedure 1 and after purification by preparative LCMS (gradient 25-45% CH₃CN in H₂O containing 10 mM NH₄HCO₃) and lyophilization from CH₃CN/H₂O, the title compound was obtained as a solid (45.38 mg, 40.4%). HPLC purity: >99% (215 nm), >99% (254 nm), >99% (280 nm); Rₜ: 1.02 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.17 (s, 3H), 1.19 (s, 3H), 1.98 (s, 3H), 3.00-3.50 (m, 4H), 3.55-3.72 (m, 4H), 4.00-4.20 (m, 2H), 4.35 (m, 1H), 4.77 (d, J=5.9 Hz, 2H), 7.58 (m, 1H), 7.71 (m, 1H), 7.94 (d, J=8.2 Hz, 1H), 7.99 (d, J=8.2 Hz, 1H), 8.18 (dd, J=5.4, 4.1 Hz, 1H), 8.27 (d, J=1.6 Hz, 1H). M.S. 460.2 (ESI) (MH⁺).

### Example 344

### (R)-2-(4-acetylpiperazin-1-yl)-6-isopropyl-4-(1-(quinolin-3-yl)ethylamino)-5H-pyrrolo[3,4-d]pyrimidin-7(6H)-one (Enantiomer 1) and

### Example 345

### (S)-2-(4-acetylpiperazin-1-yl)-6-isopropyl-4-(1-(quinolin-3-yl)ethylamino)-5H-pyrrolo[3,4-d]pyrimidin-7(6H)-one (Enantiomer 2)

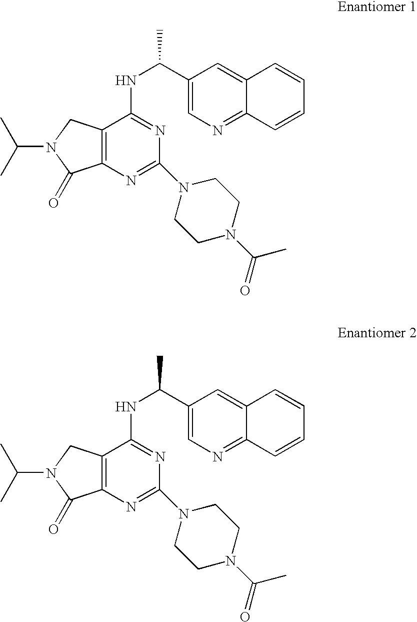

Following a procedure similar to that described in General Procedure 1, starting from 1-(quinolin-3-yl)ethanamine hydrochloride (Intermediate 142) and after purification by preparative chiral HPLC (15% isopropanol/heptane; small OD column) and lyophilization from CH₃CN/H₂O, two enantiomers were separated.

(R)-2-(4-acetylpiperazin-1-yl)-6-isopropyl-4-(1-(quinolin-3-yl)ethylamino)-5H-pyrrolo[3,4-d]pyrimidin-7(6H)-one (Enantiomer 1) was obtained as a solid (102 mg, 59.8%). Chiral SFC purity: 96% (OJ Column with MeOH+0.1% DMEA Iso at 20%). HPLC purity: >96% (215 nm), >96% (254 nm), >97% (280 nm); Rₜ: 1.07 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (dd, J=7.0, 2.4 Hz, 6H), 1.61 (d, J=7.0 Hz, 3H), 1.96 (s, 3H), 3.30-3.10 (m, 4H), 3.70-3.43 (m, 4H), 4.19 (d, J=2.4 Hz, 2H), 4.36 (m, 1H), 5.40 (m, 1H), 7.58 (m, 1H), 7.69 (m, 1H), 7.96 (m, 1H), 8.03 (d, J=7.0 Hz, 1H), 8.28 (d, J=2.3 Hz, 1H), 8.98 (d, J=2.3 Hz, 1H). M.S. 474.2 (ESI) (MH⁺).

(S)-2-(4-acetylpiperazin-1-yl)-6-isopropyl-4-(1-(quinolin-3-yl)ethylamino)-5H-pyrrolo[3,4-d]pyrimidin-7(6H)-one (Enantiomer 2) was obtained as a solid (45.5 mg, 26.7%). Chiral SFC purity: 96% (OJ Column with MeOH+0.1% DMEA Iso at 20%). HPLC purity: >96% (215 nm), >96% (254 nm), >97% (280 nm); Rₜ: 1.07 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.20 (dd, J=7.0, 2.3 Hz, 6H), 1.61 (d, J=7.0 Hz, 3H), 1.96 (s, 3H), 3.30-3.11 (m, 4H), 3.70-3.44 (m, 4H), 4.19 (d, J=2.3 Hz, 2H), 4.36 (m, 1H), 5.40 (m, 1H), 7.58 (m, 1H), 7.69 (m, 1H), 7.96 (m, 1H), 8.03 (d, J=7.0 Hz, 1H), 8.28 (d, J=2.3 Hz, 1H), 8.98 (d, J=2.3 Hz, 1H). M.S. 474.2 (ESI) (MH⁺).

### Example 346

### 2-(4-acetylpiperazin-1-yl)-6-isopropyl-4-(methyl(quinolin-3-ylmethyl)amino)-5H-pyrrolo[3,4-d]pyrimidin-7(6H)-one

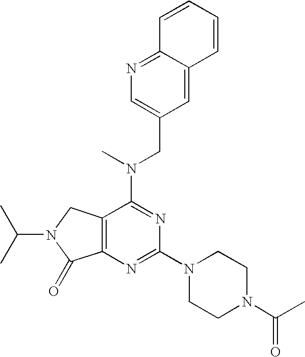

Following a procedure similar to that described in General Procedure 1, starting from N-methyl-1-(quinolin-3-yl)methanamine (Intermediate 143) and after purification by preparative LCMS (gradient 25-45% CH₃CN in H₂O containing 10 mM NH₄HCO₃) and lyophilization from CH₃CN/H₂O, the title compound was obtained as a solid (121 mg, 41.9%). HPLC purity: >96% (215 nm), >95% (254 nm), >95% (280 nm); Rₜ: 2.14 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.18 (d, J=7.0 Hz, 6H), 1.97 (s, 3H), 3.32 (s, 3H), 3.32-3.43 (m, 4H), 3.75-3.55 (m, 4H), 4.37 (m, 1H), 4.61 (s, 2H), 5.05 (s, 2H), 7.57 (m, 1H), 7.72 (m, 1H), 7.94 (m, 1H), 8.00 (d, J=8.2 Hz, 1H), 8.20 (d, J=1.6 Hz, 1H), 8.87 (d, J=2.3 Hz, 1H). M.S. 474.57 (ESI) (MH⁺). HRMS m/z calcd for C₂₆H₃₂N₇O₂ [M+H]⁺ 474.2539, found 474.2611.

### Example 347

### 2-(4-acetylpiperazin-1-yl)-6-isopropyl-4-(methyl((2-methylquinolin-3-yl)methyl)amino)-5H-pyrrolo[3,4-d]pyrimidin-7(6H)-one

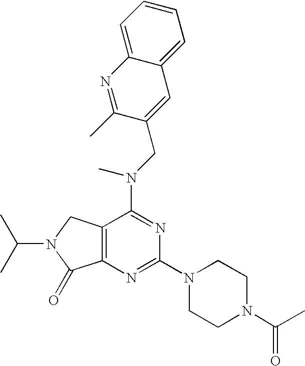

Following a procedure similar to that described in General Procedure 1, starting from N-methyl-1-(2-methylquinolin-3-yl)methenamine (Intermediate 144) and after purification by preparative LCMS (gradient 25-45% CH₃CN in H₂O containing 10 mM NH₄HCO₃) and lyophilization from CH₃CN/H₂O, the title compound was obtained as a solid (67.00 mg, 18.79%). HPLC purity: >99% (215 nm), >99% (254 nm), >99% (280 nm); Rₜ: 1.05 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.18 (d, J=6.3 Hz, 6H), 1.95 (s, 3H), 2.67 (s, 3H), 3.35-3.20 (m, 5H), 3.70-3.50 (m, 4H), 4.37 (m, 1H), 4.60 (s, 2H), 5.00 (s, 2H), 7.48 (t, J=7.4 Hz, 1H), 7.66 (t, J=7.4 Hz, 1H), 7.90 (m, 3H). M.S. 488.3 (ESI) (MH⁺). HRMS m/z calcd for C₂₇H₃₄N₇O₂ [M+H]⁺ 488.2696, found 488.2768.

### Example 348

### 2-(4-acetylpiperazin-1-yl)-4-(((6-chloroisoquinolin-3-yl)methyl)(methyl)amino)-6-isopropyl-5H-pyrrolo[3,4-d]pyrimidin-7(6H)-one

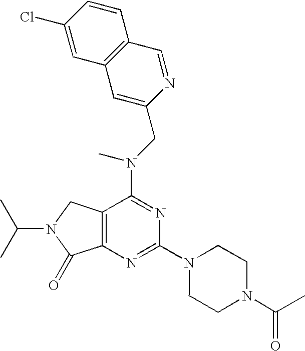

Following a procedure similar to that described in General Procedure 1, starting from 1-(6-chloroisoquinolin-3-yl)-N-methylmethanamine (Intermediate 145) and after purification by preparative LCMS (gradient 30-50% CH₃CN in H₂O containing 10 mM NH₄HCO₃) and lyophilization from CH₃CN/H₂O, the title compound was obtained as a solid (62.0 mg, 30.0%). HPLC purity: >95% (215 nm), >98% (254 nm), >99% (280 nm); Rₜ: 1.60 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (d, J=6.6 Hz, 6H), 1.97 (s, 3H), 3.32 (s, 3H), 3.30-3.42 (m, 5H), 3.50-3.71 (m, 4H), 4.32-4.45 (m, 1H), 4.60 (s, 2H), 5.02 (s, 2H), 7.66 (dd, J=8.6, 1.8 Hz, 1H), 7.69 (s, 1H), 8.09 (d, J=1.8 Hz, 1H), 8.16 (d, J=9.0 Hz, 1H), 9.32 (s, 1H). M.S. 508.3 (ESI) (MH⁺). HRMS m/z calcd for C₂₆H₃₁ClN₇O₂ [M+H]⁺ 508.2222, found 508.2220.

### Example 349

### 2-(4-acetylpiperazin-1-yl)-4-(((6-fluoroisoquinolin-3-yl)methyl)(methyl)amino)-6-isopropyl-5H-pyrrolo[3,4-d]pyrimidin-7(6H)-one

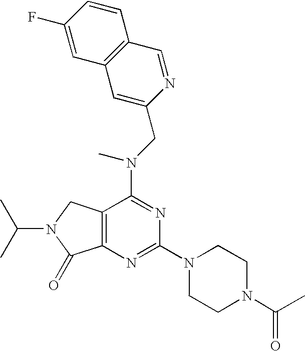

Following a procedure similar to that described in General Procedure 1, starting from 1-(6-fluoroisoquinolin-3-yl)-N-methylmethanamine (Intermediate 146) and after purification by preparative LCMS (gradient 30-50% CH₃CN in H₂O containing 10 mM NH₄HCO₃) and lyophilization from CH₃CN/H₂O, the title compound was obtained as a solid (63.0 mg, 30.9%). HPLC purity: >96% (215 nm), >97% (254 nm), >96% (280 nm); Rₜ: 1.36 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (d, J=6.6 Hz, 6H), 1.97 (s, 3H), 3.31 (s, 3H), 3.32-3.43 (m, 5H), 3.52-3.71 (m, 4H), 4.32-4.40 (m, 1H), 4.56-4.65 (m, 2H), 5.02 (s, 2H), 7.54 (d, J=2.4 Hz, 1H), 7.69 (s, 1H), 7.74 (dd, J=9.0, 2.3 Hz, 1H), 8.10 (dd, J=9.0, 5.9 Hz, 1H), 9.31 (s, 1H). M.S. 492.3 (ESI) (MH⁺). HRMS m/z calcd for C₂₆H₃₁FN₇O₂ [M+H]⁺ 492.2445, found 492.2516.

### Example 350

### 2-(4-acetylpiperazin-1-yl)-4-(((7-fluoroisoquinolin-3-yl)methyl)(methyl)amino)-6-isopropyl-5H-pyrrolo[3,4-d]pyrimidin-7(6H)-one

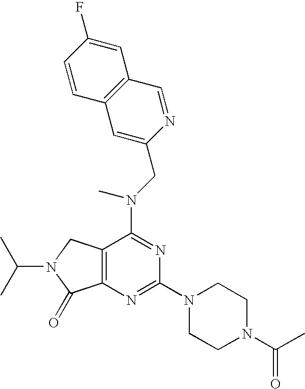

Following a procedure similar to that described in General Procedure 1, starting from 1-(7-fluoroisoquinolin-3-yl)-N-methylmethanamine (Intermediate 147) and after purification by preparative LCMS (gradient 30-50% CH₃CN in H₂O containing 10 mM NH₄HCO₃) and lyophilization from CH₃CN/H₂O, the title compound was obtained as a solid (26.0 mg, 14.37%). HPLC purity: >99% (215 nm), >98% (254 nm), >99% (280 nm); Rₜ: 1.43 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (d, J=6.6 Hz, 6H), 1.97 (s, 3H), 3.30-3.42 (m, 5H), 3.34 (s, 3H), 3.52-3.70 (m, 4H), 4.30-4.45 (m, 1H), 4.61 (s, 2H), 5.03 (s, 2H), 7.69 (d, J=2.7 Hz, 1H), 7.76 (s, 1H), 7.92 (dd, J=9.0, 2.7 Hz, 1H), 8.05 (dd, J=9.0, 5.5 Hz, 1H), 9.29 (s, 1H). M.S. 492.3 (ESI) (MH⁺). HRMS m/z calcd for C₂₆H₃₁FN₇O₂ [M+H]⁺ 492.2445, found 492.2515.

### Example 351

### (R)-4-(1-(2-(4-acetylpiperazin-1-yl)-6-isopropyl-7-oxo-6,7-dihydro-5H-pyrrolo[3,4-d]pyrimidin-4-ylamino)ethyl)benzonitrile

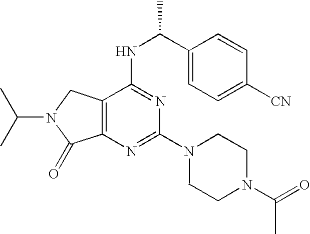

Following a procedure similar to that described in General Procedure 1, starting from (R)-4-(1-aminoethyl)benzonitrile hydrochloride (Intermediate 148) and after purification by silica gel chromatography (0-10% MeOH/DCM) followed by preparative HPLC purification (high pH: Phenomenex Gemini C18, 21.2×250 mm, 5 μm particle size, gradient 45-65% CH₃CN in H₂O containing 10 mM NH₄HCO₃, retention time 8.87 min), the title compound (0.139 g, 38.8%) was obtained as a solid. [α]_{D}=+99.2 (c=0.007, MeOH). HPLC purity: >98% (215 nm), >98% (254 nm), >99% (280 nm); Rₜ: 1.34 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CDCl₃) δ ppm ¹H NMR (400 MHz, CDCl₃), δ ppm 1.27 (dd, J=6.64, 1.56 Hz, 6H), 1.60 (d, J=7.03 Hz, 3H), 2.12 (s, 3H), 3.31-3.44 (m, 2H), 3.46-3.83 (m, 6H), 4.06-4.19 (m, 2H), 4.68 (quin, J=6.74 Hz, 1H), 4.85 (d, J=5.86 Hz, 1H), 5.22-5.32 (m, 1H), 7.45 (d, J=8.20 Hz, 2H), 7.63 (d, J=8.20 Hz, 2H). M.S. 448.2 (ESI) (MH⁺). HRMS m/z calcd for C₂₄H₃₀N₇O₂ [M+H]⁺ 448.24555, found 448.24569.

### Example 352

### 2-(4-Acetyl-2-methylpiperazin-1-yl)-6-isopropyl-4-(quinolin-3-ylmethylamino)-5H-pyrrolo[3,4-d]pyrimidin-7(6M)-one

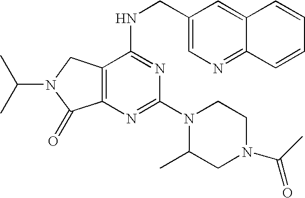

Following a procedure similar to that described in General Procedure 1, starting from 1-(3-methyl-piperazin-1-yl)-ethanone hydrochloride (Intermediate 151), the title compound was obtained as a solid. HPLC: 96.65%, Rₜ: 9.014 minutes. ¹H NMR (400 MHz, CDCl₃) δ ppm 0.92-1.16 (m, 3H) 1.25 (d, J=6.63 Hz, 6H) 2.11 (d, J=16.00 Hz, 3H) 2.59-2.76 (m, 0.5H) 2.93 (dd, J=13.27, 3.51 Hz, 0.5H) 3.05-3.26 (m, 1H) 3.33 (dd, J=13.27, 3.51 Hz, 0.5H) 3.54 (d, J=13.27 Hz, 0.5H) 3.70 (d, J=9.37 Hz, 0.5H) 4.12 (s, 2H) 4.32 (d, J=13.27 Hz, 0.5H) 4.54 (d, J=13.27 Hz, 1H) 4.60-4.77 (m, 2H) 4.83-5.08 (m, 3H) 5.18 (d, J=4.68 Hz, 1H) 7.57 (t, J=7.42 Hz, 1H) 7.73 (t, J=7.42 Hz, 1H) 7.78 (d, J=8.20 Hz, 1H) 7.99-8.19 (m, 2H) 8.93 (br. s., 1H). M.S. (calcd): 472.58 (MH⁺), M.S. (found): 472.66 (ESI) (MHz).

### Example 353

### 2-(4-acetylpiperazin-1-yl)-6-isopropyl-4-(methyl((7-methylisoquinolin-3-yl)methyl)amino)-5H-pyrrolo[3,4-d]pyrimidin-7(6H)-one

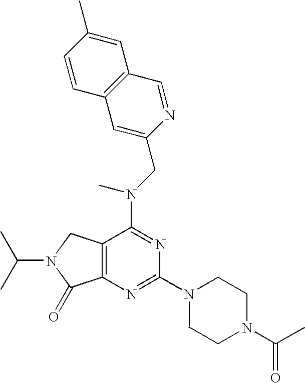

Following a procedure similar to that described in General Procedure 1, starting from N-methyl-1-(7-methylisoquinolin-3-yl)methanamine (Intermediate 152) and after purification by preparative LCMS (gradient 25-45% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (3.5 mg, 13.4%). HPLC purity: >98% (215 nm), >98% (254 nm), >97% (280 nm); Rₜ: 1.31 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.19 (d, J=6.7 Hz, 6H), 1.98 (s, 3H), 2.48 (s, 3H), 3.30-3.45 (m, 4H), 3.54-3.73 (m, 4H), 4.30-4.45 (m, 1H), 4.61 (d, 2H), 5.00 (s, 2H), 7.47 (dd, J=8.2, 1.6 Hz, 1H), 7.58 (s, 1H), 7.69 (s, 1H), 7.99 (d, J=8.2 Hz, 1H), 9.20 (s, 1H). M.S. 488.3. (ESI) (MH⁺). HRMS m/z calcd for C₂₇H₃₃N₇O₂ [M+H]⁺ 488.2769, found 488.2765.

### Example 354

### 2-(4-acetylpiperazin-1-yl)-6-(1-methylethyl)-4-{[(1-methyl-1H-indol-5-yl)methyl]amino}-5,6-dihydro-7H-pyrrolo[3,4-d]pyrimidin-7-one

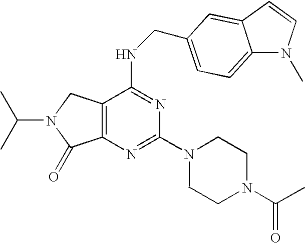

Following a procedure similar to that described in General Procedure 1 and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (54 mg, 29.2%). HPLC Purity: >97% (215 nm), >97% (254 nm), >98% (280 nm); Rₜ: 1.489 minutes. ¹H NMR (400 MHz, CD₃OD) δ ppm 1.26 (d, J=6.64 Hz, 6H), 2.08 (s, 3H), 3.41-3.47 (m, 2H), 3.49-3.55 (m, 2H), 3.74 (s, 3H), 3.75-3.81 (m, 2H), 3.81-3.87 (m, 2H), 4.16 (s, 2H), 4.38-4.55 (m, 1H), 4.72 (s, 2H), 6.34 (d, J=2.73 Hz, 1H), 7.09 (d, J=2.73 Hz, 1H), 7.14-7.18 (m, 1H), 7.28 (d, J=8.59 Hz, 1H) 7.51 (s, 1H). M.S. (found): 462.3 (ESI) (MH⁺); HRMS m/z calcd for C₂₅H₃₂N₇O₂ [M+H]⁺ 462.26120.

### Example 355

### 2-(4-acetylpiperazin-1-yl)-6-(1-methylethyl)-4-{methyl[(1-methyl-1H-indol-6-yl)methyl]amino}-5,6-dihydro-7H-pyrrolo[3,4-d]pyrimidin-7-one

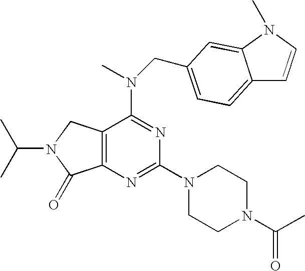

Following a procedure similar to that described in General Procedure 1 and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (74 mg, 38.9%). HPLC Purity: >98% (215 nm), >99% (254 nm), >99% (280 nm); Rₜ: 1.666 minutes. ¹H NMR (400 MHz, CD₃OD) δ ppm 1.23 (d, J=6.64 Hz, 6H), 2.09 (s, 3H), 3.22 (s, 3H), 3.46-3.53 (m, 2H), 3.54-3.59 (m, 2H), 3.73 (s, 3H), 3.77-3.83 (m, 2H), 3.84-3.90 (m, 2H), 4.47 (dt, J=13.38, 6.79 Hz, 1H), 4.54 (s, 2H), 4.96 (s, 2H), 6.36 (d, J=2.73 Hz, 1H), 6.93 (d, J=8.20 Hz, 1H), 7.10 (d, J=3.12 Hz, 1H), 7.23 (s, 1H), 7.47 (d, J=8.20 Hz, 1H). M.S. (found): 476.2 (ESI) (MH⁺); HRMS m/z calcd for C₂₆H₃₄N₇O₂ [M+H]⁺ 476.27685.

### Example 356

### 2-(4-acetylpiperazin-1-yl)-6-(1-methylethyl)-4-{[(1-methyl-1H-indol-6-yl)methyl]amino}-5,6-dihydro-7H-pyrrolo[3,4-d]pyrimidin-7-one

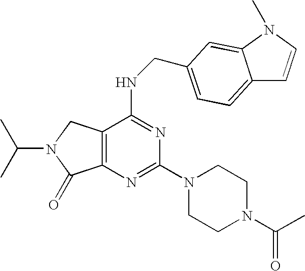

Following a procedure similar to that described in General Procedure 1 and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (78 mg, 42.2%). HPLC Purity: >99% (215 nm), >99% (254 nm), >99% (280 nm); Rₜ: 1.515 minutes. ¹H NMR (400 MHz, CD₃OD) δ ppm 1.26 (d, J=6.64 Hz, 6H), 2.08 (s, 3H), 3.41-3.47 (m, 2H), 3.49-3.55 (m, 2H), 3.74 (s, 3H), 3.76-3.81 (m, 2H), 3.81-3.86 (m, 2H), 4.18 (s, 2H), 4.49 (quin, J=6.64 Hz, 1H), 4.76 (s, 2H), 6.34 (d, J=2.73 Hz, 1H), 7.04 (d, J=8.20 Hz, 1H), 7.08 (d, J=3.13 Hz, 1H), 7.34 (s, 1H), 7.45 (d, J=8.20 Hz, 1H). M.S. (found): 462.3 (ESI) (MH⁺); HRMS m/z calcd for C₂₅H₃₂N₇O₂ [M+H]⁺ 462.26120, found 462.26090.

### Example 357

### (R)-2-(4-acetylpiperazin-1-yl)-4-(1-(2,4-diethoxyphenyl)ethylamino)-6-isopropyl-5H-pyrrolo[3,4-d]pyrimidin-7(6H)-one

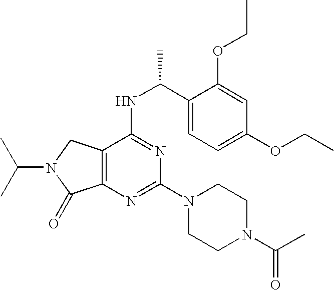

Following a procedure similar to that described in General Procedure 1, starting from (R)-1-(2,4-diethoxyphenyl)ethanamine hydrochloride (Intermediate 153) and after purification by preparative LCMS (gradient 45-65% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (43 mg, 51.7%). [α]_{D}=+47.5 (c=0.01, MeOH). Purity: >99% (215 nm), >99% (254 nm), >99% (280 nm); Rₜ: 1.87 minutes; Conditions: Zorbax SB C-18; Gradient: 05-95% B in 4.5 min, 70° C. A: 0.05% TFA in H₂O, B: 0.05% TFA in CH₃CN. ¹H NMR (400 MHz, CD₃OD) δ ppm 1.25 (d, J=6.64 Hz, 6H), 1.41 (t, J=7.03 Hz, 3H), 1.46 (t, J=6.84 Hz, 3H), 1.54 (d, J=6.64 Hz, 3H), 2.14 (s, 3H), 3.46 (t, J=5.08 Hz, 2H), 3.57-3.71 (m, 2H), 3.83 (t, J=4.88 Hz, 2H), 3.88-3.94 (m, 2H), 3.98-4.16 (m, 6H), 4.66 (quin, J=6.74 Hz, 1H), 5.32-5.37 (m, 1H), 5.41 (br. s., 1H), 6.42 (dd, J=8.20, 2.34 Hz, 1H), 6.48 (d, J=2.34 Hz, 1H), 7.12 (d, J=8.20 Hz, 1H). MS [M+H]⁺ 511.2 (ESI). HRMS m/z calcd for C₂₇H₃₉N₆O₄ [M+H]⁺ 511.30273, found 511.30273.

### Example 358

### (R)-2-(4-acetylpiperazin-1-yl)-4-(1-(4-((dimethylamino)methyl)phenyl)ethylamino)-6-isopropyl-5H-pyrrolo[3,4-d]pyrimidin-7(6H)-one

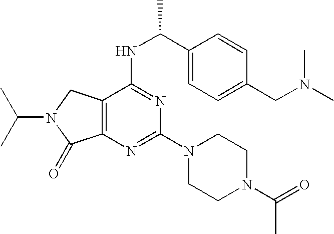

To a solution of (R)-2-(4-acetylpiperazin-1-yl)-4-(1-(4-(hydroxymethyl)phenyl)ethylamino)-6-isopropyl-5H-pyrrolo[3,4-d]pyrimidin-7(6H)-one (Example 332, 141 mg, 0.31 mmol) and triphenylphosphine (82 mg, 0.31 mmol) in THF (3 mL) at −18° C. was added N-bromosuccinimide (55.5 mg, 0.31 mmol) and the reaction mixture was stirred at −18° C. for 10 minutes. A 2.0 M solution of dimethylamine (0.389 mL, 0.78 mmol) in THF was added and the reaction mixture was warmed to rt and then heated to reflux for 2 h, cooled to rt and concentrated under reduced pressure, the residue was purified by preparative HPLC (high pH, X-Bridge Prep C18 OBD, 30×50 mm, 5 μm particle size, 30-50% ACN in water) to give the title compound (35.8 mg, 24.0%) as a solid. [α]_{D}=+131.0 (c=0.01, MeOH). HPLC purity: >94% (215 nm), >96% (254 nm), >97% (280 nm); Rₜ: 0.92 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.64 Hz, 6H), 1.47 (d, J=6.64 Hz, 3H), 2.01 (s, 3H), 2.10 (s, 6H), 3.32 (br. s., 2H), 3.33-3.46 (m, 4H), 3.49-3.77 (m, 4H), 4.15 (s, 2H), 4.36 (quin, J=6.74 Hz, 1H), 5.19 (t, J=7.03 Hz, 1H), 7.20 (d, J=7.81 Hz, 2H), 7.33 (d, J=8.20 Hz, 2H), 7.83 (d, J=7.42 Hz, 1H). M.S. 480.2 (ESI) (MH⁺). HRMS m/z calcd for C₂₆H₃₈N₇O₂ [M+H]⁺ 480.30815, found 480.30735.

### Example 359

### (R)-2-(4-acetylpiperazin-1-yl)-4-(1-(2,2-difluorobenzo[d][1,3]dioxol-5-yl)ethylamino)-6-isopropyl-5H-pyrrolo[3,4-d]pyrimidin-7(6H)-one

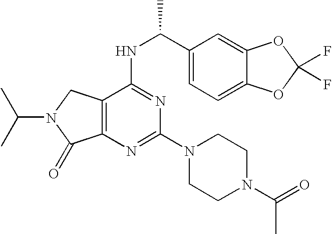

Following a procedure similar to that described in General Procedure 1, starting from (R)-1-(2,2-difluorobenzo[d][1,3]dioxol-5-yl)ethanamine (Intermediate 154) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (216 mg, 70.5% over 2 steps). [α]_{D}=+132.7 (c=0.01, MeOH). HPLC purity: >99% (215 nm), >99% (254 nm), >99% (280 nm); Rₜ: 1.79 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.21 (d, J=6.64 Hz, 6H), 1.47 (d, J=7.03 Hz, 3H), 2.01 (s, 3H), 3.19-3.46 (m, 4H), 3.47-3.79 (m, 4H), 4.17 (br. s., 2H), 4.37 (quin, J=6.64 Hz, 1H), 5.13-5.30 (m, 1H), 7.20-7.27 (m, 1H), 7.30-7.38 (m, 1H), 7.44 (s, 1H), 7.86 (d, J=7.03 Hz, 1H). M.S. 503.3 (ESI) (MH⁺). HRMS m/z calcd for C₂₄H₂₉F₂N₆O₄ [M+H]⁺ 503.22129, found 503.22062.

### Example 360

### 2-(4-acetylpiperazin-1-yl)-4-(((2,2-difluorobenzo[d][1,3]dioxol-5-yl)methyl)(methyl)amino)-6-isopropyl-5H-pyrrolo[3,4-d]pyrimidin-7(6H)-one

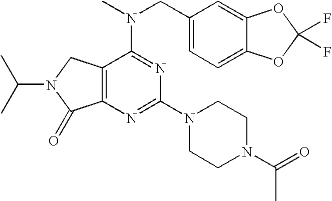

Following a procedure similar to that described in General Procedure 1, starting from 1-(2,2-difluorobenzo[d][1,3]dioxol-5-yl)-N-methylmethanamine (Intermediate 155) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (56.5 mg, 10.0% over 2 steps). HPLC purity: >99% (215 nm), >99% (254 nm), >99% (280 nm); Rₜ: 1.73 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.64 Hz, 6H), 2.02 (s, 3H), 3.22 (s, 3H), 3.42 (br. s., 4H), 3.63 (d, J=5.47 Hz, 2H), 3.70 (d, J=5.08 Hz, 2H), 4.38 (quin, J=6.74 Hz, 1H), 4.57 (s, 2H), 4.85 (s, 2H), 7.14 (d, J=7.42 Hz, 1H), 7.33 (s, 1H), 7.37 (d, J=8.20 Hz, 1H). M.S. 503.3 (ESI) (MH⁺). HRMS m/z calcd for C₂₄H₂₉F₂N₆O₄ [M+H]⁺ 503.22129, found 503.22063.

### Example 361

### (R)-4-(1-(4-ethoxyphenyl)ethylamino)-2-(4-ethyl-3-oxopiperazin-1-yl)-6-isopropyl-5H-pyrrolo[3,4-d]pyrimidin-7(6H)-one

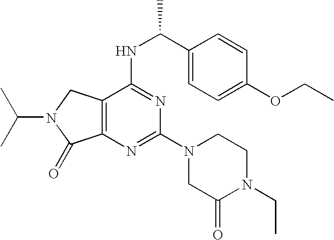

Following a procedure similar to that described in General Procedure 1, starting from 1-ethylpiperazin-2-one (Intermediate 156) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (142 mg, 49.9% over 2 steps). HPLC purity: >99% (215 nm), >99% (254 nm), >99% (280 nm); Rₜ: 1.78 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.00 (t, J=7.03 Hz, 3H), 1.20 (d, J=6.64 Hz, 6H), 1.29 (t, J=7.03 Hz, 3H), 1.46 (d, J=7.03 Hz, 3H), 3.11-3.33 (m, 4H), 3.77-3.93 (m, 2H), 3.97 (q, J=6.90 Hz, 2H), 4.02-4.28 (m, 4H), 4.36 (quin, J=6.64 Hz, 1H), 5.19 (t, J=6.84 Hz, 1H), 6.84 (d, J=8.59 Hz, 2H), 7.29 (d, J=8.59 Hz, 2H), 7.84 (d, J=7.42 Hz, 1H). M.S. 467.3 (ESI) (MH⁺). HRMS m/z calcd for C₂₅H₃₅N₆O₃ [M+H]⁺ 467.27652, found 467.27653.

### Example 362

### 2-(4-ethyl-3-oxopiperazin-1-yl)-6-isopropyl-4-((isoquinolin-3-ylmethyl)(methyl)amino)-5H-pyrrolo[3,4-d]pyrimidin-7(6H)-one

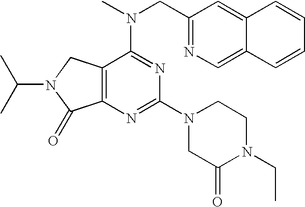

Following a procedure similar to that described in General Procedure 1, starting from 1-(isoquinolin-3-yl)-N-methylmethanamine (Intermediate 70) and 1-ethylpiperazin-2-one (Intermediate 156) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (20.0 mg, 31.0% over 2 steps). HPLC purity: >99% (215 nm), >99% (254 nm), >99% (280 nm); Rₜ: 1.32 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, CD₃OD) δ ppm 1.03-1.10 (m, 3H), 1.30 (d, J=6.64 Hz, 6H), 3.43 (s, 3H), 3.73 (s, 2H), 3.98 (br. s., 2H), 4.29 (s, 2H), 4.48-4.58 (m, 1H), 4.69 (s, 2H), 5.11 (s, 2H), 5.49 (s, 2H), 7.65 (d, J=8.59 Hz, 1H), 7.69 (d, J=10.55 Hz, 1H), 7.72-7.79 (m, 1H), 7.88 (d, J=4.30 Hz, 1H), 8.10 (d, J=8.98 Hz, 1H), 9.24 (s, 1H). M.S. 474.2 (ESI) (MH⁺). HRMS m/z calcd for C₂₆H₃₂N₇O₂ [M+H]⁺ 474.26120, found 474.26057.

### Example 363

### (R)-4-(1-(4-ethoxyphenyl)ethylamino)-6-isopropyl-2-(3-oxo-4-(2,2,2-trifluoroethyl)piperazin-1-yl)-5H-pyrrolo[3,4-d]pyrimidin-7(6H)-one

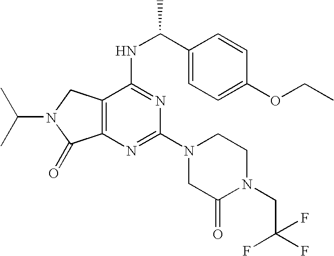

Following a procedure similar to that described in General Procedure 1, starting from 1-(2,2,2-trifluoroethyl)piperazin-2-one (Intermediate 157) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (69.8 mg, 22.0% over 2 steps). HPLC purity: >96% (215 nm), >95% (254 nm), >96% (280 nm); Rₜ: 2.04 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.20 (d, J=6.64 Hz, 6H), 1.29 (t, J=7.03 Hz, 3H), 1.46 (d, J=7.03 Hz, 3H), 3.35-3.53 (m, 2H), 3.81-4.02 (m, 4H), 4.15 (s, 2H), 4.17-4.29 (m, 3H), 4.30-4.43 (m, 2H), 5.19 (t, J=6.84 Hz, 1H), 6.83 (d, J=8.59 Hz, 2 H), 7.29 (d, J=8.59 Hz, 2H), 7.87 (d, J=7.42 Hz, 1H). M.S. 521.3 (ESI) (MH⁺). HRMS m/z calcd for C₂₅H₃₂F₃N₆O₃ [M+H]⁺ 521.24825, found 521.24782.

### Example 364

### 6-isopropyl-4-((isoquinolin-3-ylmethyl)(methyl)amino)-2-(3-oxo-4-(2,2,2-trifluoroethyl)piperazin-1-yl)-5H-pyrrolo[3,4-d]pyrimidin-7(6H)-one

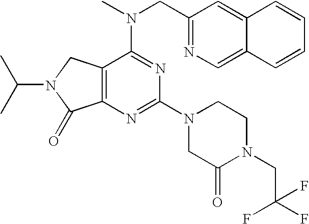

Following a procedure similar to that described in General Procedure 1, starting from 1-(isoquinolin-3-yl)-N-methylmethanamine (Intermediate 70) and 1-(2,2,2-trifluoroethyl)piperazin-2-one (Intermediate 157) and after purification by preparative LCMS (gradient 35-55% CH₃CN in H₂O containing 10 mM NH₄HCO₃) and lyophilization from CH₃CN/H₂O (column conditions: X-Bridge Prep C18 OBD, 30×50 mm, 5 μm particle size), the title compound was obtained as a solid (22.0 mg, 30.6% over 2 steps). HPLC purity: >97% (215 nm), >96% (254 nm), >97% (280 nm); Rₜ: 1.52 minutes; Conditions: Zorbax C-18, gradient 5-95% B in 4.5 min, flow rate 3.5 mL/min, 70° C., A: 0.05% TFA in H₂O, B: 0.05% TFA in CH₃CN. ¹H NMR (400 MHz, DMSO-d₆) δ ppm 1.19 (d, J=6.25 Hz, 6H), 3.03-3.16 (m, 3H), 3.57-3.64 (m, 2H), 3.89 (br. s., 2H), 4.16 (dd, J=16.41, 7.03 Hz, 2H), 4.29 (br. s., 2H), 4.34-4.42 (m, 1H), 4.63 (br. s., 2H), 5.04 (s, 2H), 7.61-7.67 (m, 1H), 7.72-7.78 (m, 1H), 7.89-7.94 (m, 1H), 8.10 (d, J=8.20 Hz, 1H), 8.32 (s, 1H), 9.29 (s, 1H). M.S. 528.3 (ESI) (MH⁺). HRMS m/z calcd for C₂₆H₂₉F₃N₇O₂ [M+H]⁺ 528.23293, found 528.23267.

1. A compound of formula I, a pharmaceutically acceptable salt thereof, a diastereomer, an enantiomer, or a mixture thereof: 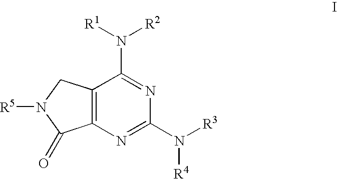
wherein:
R¹ and R² are independently selected from hydrogen, C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₃₋₇cycloalkyl fused with a phenyl and a C₂₋₆heteroaryl, C₆₋₁₀aryl fused with a C₃₋₇cycloalkyl, C₁₋₁₄heterocyclyl, C₁₋₁₄heterocyclyl-C₁₋₆alkyl, C₆₋₁₀aryl, C₆₋₁₀aryl-C₁₋₆alkyl, or R¹ and R² together with the nitrogen connected thereto form a C₂₋₉heterocyclyl; wherein said C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₃₋₇cycloalkyl fused with a phenyl and a C₂₋₆heteroaryl, C₆₋₁₀aryl fused with a C₃₋₇cycloalkyl, C₁₋₁₄heterocyclyl, C₁₋₁₄heterocyclyl-C₁₋₆alkyl, C₆₋₁₀aryl, C₆₋₁₀aryl-C₁₋₆alkyl and C₂₋₉heterocyclyl are optionally substituted by one or more groups selected from, halogen, cyano, nitro, C₁₋₆alkoxy, C₁₋₄haloalkoxy, C₁₋₆alkyl, halogenated C₁₋₆alkyl, C₃₋₆-cycloalkyl, C₃₋₆cycloalkoxy, C₃₋₆cycloalkyl-C₁₋₄alkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷—(CH₂)ₘ—C(═O)R⁷—(CH₂)ₘ—S(═O)₂R⁷, —(CH₂)ₘ—S(═O)R⁷—(CH₂)ₘ—SR⁷, —(CH₂)ₘ—O—C(═O)—R⁷—(CH₂)ₘ—OR⁷—(CH₂)ₘ—NR⁷R⁸ hydroxy, C₁₋₁₄heterocyclyl-C₀₋₄-alkyl, phenyl, benzyl, phenylethyl, wherein said C₁₋₁₄heterocyclyl-C₀₋₄-alkyl, phenyl, benzyl or phenethyl optionally substituted by one or more groups selected from halogen, cyano, nitro, oxo, C₁₋₆alkoxy, C₁₋₄haloalkoxy, C₁₋₆alkyl, halogenated C₁₋₆alkyl, C₃₋₆-cycloalkyl, C₃₋₆cycloalkoxy, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷—(CH₂)ₘ—C(═O)R⁷—(CH₂)ₘ—S(═O)₂R⁷—(CH₂)ₘ—S(═O)R⁷—(CH₂)ₘ—SR⁷—(CH₂)ₘ—O—C(═O)—R⁷—(CH₂)ₘ—R⁷, —(CH₂)ₘ—NR⁷R⁸ and hydroxy;
R³ and R⁴ are independently selected from hydrogen, C₁₋₆alkyl, C₂₋₆alkenyl, C₃₋₈cycloalkyl, C₃₋₈cycloalkyl-C₁₋₆alkyl, C₆₋₁₀aryl, C₆₋₁₀aryl-C₁₋₆alkyl, C₃₋₆heterocyclyl, and C₃₋₆heterocyclyl-C₁₋₆alkyl; or R³ and R⁴ together with the nitrogen connected thereto form a C₂₋₉heterocyclyl; wherein said C₁₋₆alkyl, C₂₋₆alkenyl, C₃₋₈cycloalkyl, C₃₋₈cycloalkyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl and C₂₋₉heterocyclyl are optionally substituted by one or more groups selected from C₁₋₆alkyl, halogenated C₁₋₆alkyl, carboxy, halogen, cyano, nitro, oxo, C₁₋₄-alkoxy, C₁₋₄haloalkoxy, hydroxy, C₃₋₆cycloalkyl-C₁₋₄alkoxy, C₃₋₆heterocycloalkyl, —(CH₂)ₘ—C₃₋₆heterocyclyl, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷—(CH₂)ₘ—C(═O)R⁷—(CH₂)ₘ—S(═O)₂R⁷, —(CH₂)ₘ—O—C(═O)—R⁷—(CH₂)ₘ—OR⁷ and —(CH₂)ₘ—NR⁷R⁸;
R⁵ is selected from hydrogen and C₁₋₆alkyl, C₃₋₇-cycloalkyl, C₁₋₆heterocyclyl, and —(CH₂)ₘ—C₆₋₁₀aryl, optionally substituted with one or more groups selected from OH, C₁₋₄alkoxy, halogenated C₁₋₄alkoxy, and halogen;
R⁷ and R⁸ are independently selected from —H, C₁₋₆alkyl, C₆₋₁₀aryl, C₆₋₁₀aryl-C₁₋₄alkyl, C₁₋₅heterocyclyl, and C₃₋₆cycloalkyl-C₀₋₄alkyl, wherein said C₁₋₆alkyl, C₆₋₁₀aryl, C₆₋₁₀aryl-C₁₋₄alkyl, C₁₋₅heterocyclyl, and C₃₋₆cycloalkyl-C₀₋₄alkyl are optionally substituted with one or more groups selected from —OH, C₁₋₄alkyl, methoxy, ethoxy and halogen; and
m is 0, 1, 2 or 3,
with a proviso that at least one of R¹, R², R³ and R⁴ is not hydrogen with a further proviso that the compound is not selected from 5-[2-(4-{4-[(4-chlorophenyl)amino]-6-isopropyl-7-oxo-6,7-dihydro-5H-pyrrolo[3,4-d]pyrimidin-2-yl}piperazin-1-yl)-2-oxoethyl]imidazolidine-2,4-dione
2-amino-4-anilino-5,6-dihydro-7H-pyrrolo[3,4-d]pyrimidin-7-one;
1-[4-[(4-chlorophenyl)amino]-6,7-dihydro-6-(1-methylethyl)-7-oxo-5H-pyrrolo[3,4-d]pyrimidin-2-yl]-4-[2-(hydroxyimino)-1-oxopropyl]-piperazine;
4-[(4-chlorophenyl)amino]-5,6-dihydro-2-(4-morpholinyl)-6-[2-(4-morpholinyl)ethyl]-7H-pyrrolo[3,4-d]pyrimidin-7-one;
4-[(2-aminophenyl)amino]-5,6-dihydro-5-imino-6-propyl-2-(1-pyrrolidinyl)-7H-pyrrolo[3,4-d]pyrimidin-7-one;
5,6-dihydro-5-imino-6-propyl-4-(propylamino)-2-(1-pyrrolidinyl)-7H-pyrrolo[3,4-d]pyrimidin-7-one;
5-amino-2-(1-piperidinyl)-4-(propylamino)-7H-pyrrolo[3,4-d]pyrimidin-7-one;
4,5-diamino-2-(1-piperidinyl)-7H-Pyrrolo[3,4-d]pyrimidin-7-one;
5-amino-4-(1-piperidinyl)-2-(1-pyrrolidinyl)-7H-pyrrolo[3,4-d]pyrimidin-7-one;
5-amino-4-(propylamino)-2-(1-pyrrolidinyl) 7H-pyrrolo[3,4-d]pyrimidin-7-one;
4,5-diamino-2-(1-pyrrolidinyl)-7H-pyrrolo[3,4-d]pyrimidin-7-one;
N-[6,7-dihydro-7-oxo-6-phenyl-4-(1-piperidinyl)-5H-pyrrolo[3,4-d]pyrimidin-2-yl]-N-phenylbenzamide;
5-[(dimethylamino)methylene]-5,6-dihydro-6-phenyl-2-(phenylamino)-4-(1-piperidinyl)-7H-pyrrolo[3,4-d]pyrimidin-7-one;
2,4-bis(dimethylamino)-5,6-dihydro-6-phenyl-7H-pyrrolo[3,4-d]pyrimidin-7-one;
5,6-dihydro-6-phenyl-2-(phenylamino)-4-(1-piperidinyl)-7H-pyrrolo[3,4-d]pyrimidin-7-one;
5,6-dihydro-6-methyl-2,4-di-1-piperidinyl-7H-pyrrolo[3,4-d]pyrimidin-7-one;
4-anilino-6-butyl-2-(butylamino)-5,6-dihydro-7H-pyrrolo[3,4-d]pyrimidin-7-one;
4-anilino-2-p-anisidino-5,6-dihydro-6-(p-methoxyphenyl)-7H-pyrrolo[3,4-d]pyrimidin-7-one;
2-anilino-5,6-dihydro-4-(p-hydroxyanilino)-6-phenyl-7H-pyrrolo[3,4-d]pyrimidin-7-one;
2,4-dianilino-5,6-dihydro-6-phenyl-7H-pyrrolo[3,4-d]pyrimidin-7-one;
4-[(2-chloroethyl)amino]-5,6-dihydro-6-phenyl-2-piperidino-7H-pyrrolo[3,4-d]pyrimidin-7-one;
4-anilino-5,6-dihydro-6-(p-methoxyphenyl)-2-morpholino-7H-pyrrolo[3,4-d]pyrimidin-7-one;
4-anilino-5,6-dihydro-6-(p-methoxyphenyl)-2-piperidino-7H-pyrrolo[3,4-d]pyrimidin-7-one;
5,7-dihydro-2,4-dimorpholino-7-oxo-6H-Pyrrolo[3,4-d]pyrimidine-6-acetic acid 2-hydroxyethyl ester;
6-benzyl-5,6-dihydro-2,4-dimorpholino-7H-pyrrolo[3,4-d]pyrimidin-7-one;
5,6-dihydro-2,4-dimorpholino-6-phenyl-7H-pyrrolo[3,4-d]pyrimidin-7-one;
5,6-dihydro-6-(2-hydroxyethyl)-2,4-dipiperidino-7H-pyrrolo[3,4-d]pyrimidin-7-one; and
5,6-dihydro-6-phenyl-2,4-di-1-piperidinyl-7H-pyrrolo[3,4-d]pyrimidin-7-one.

2. A compound as claimed in claim 1, wherein
R¹ is hydrogen or C₁₋₄alkyl; and
R² is C₂₋₁₀heteroaryl-C₁₋₄alkyl, C₃₋₆heterocycloalkyl, or C₆₋₁₀aryl-C₁₋₄alkyl, wherein said C₂₋₁₀heteroaryl-C₁₋₄alkyl, C₃₋₆heterocycloalkyl, and C₆₋₁₀aryl-C₁₋₄alkyl are optionally substituted with one or more groups selected from halogen, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R³, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—C(═O)—R⁷, —(CH₂)ₘ—OR⁷, —(CH₂)ₘ—NR⁷R⁸, hydroxy, phenyl, benzyl, phenylethyl, halogenated phenyl, halogenated benzyl and halogenated phenylethyl, wherein said phenyl, benzyl, phenylethyl, halogenated phenyl, halogenated benzyl and halogenated phenylethyl are optionally substituted with one or more groups selected from —OH, methoxy, ethoxy, halogenated C₁₋₆alkyl, and C₁₋₆alkyl, wherein said R⁷ and R³ are independently selected from —H, C₁₋₆alkyl, C₆₋₁₀aryl, C₁₋₅heterocyclyl, and C₃₋₆cycloalkyl-C₀₋₄alkyl, wherein said C₁₋₆alkyl, C₆₋₁₀aryl, C₁₋₅heterocyclyl, and C₃₋₆cycloalkyl-C₀₋₄alkyl used in defining R⁷ and R⁸ are optionally substituted with one or more groups selected from —OH, methoxy, ethoxy and halogen.

3. A compound as claimed in claim 1, wherein
R¹ is hydrogen or C₁₋₄alkyl; and R² is selected from cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl, wherein said cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl are optionally substituted with one or more groups selected from halogen, cyano, nitro, C₁₋₄alkoxy, C₁₋₆alkyl, halogenated C₁₋₆alkyl, C₃₋₆heterocycloalkyl, C₃₋₆cycloalkyl, C₃₋₆cycloalkyl-C₁₋₄alkoxy, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸—(CH₂)ₘ—N(R⁷)C(═O)—OR⁸—(CH₂)ₘ—C(═O)—OR⁷—(CH₂)ₘ—C(═O)R⁷, —(CH₂)ₘ—S(═O)₂R⁷, —(CH₂)ₘ—S(═O)R⁷—(CH₂)ₘ—SR⁷—(CH₂)ₘ—O—C(═O)—R⁷—(CH₂)ₘ—OR⁷—(CH₂)ₘ—NR⁷R⁸ hydroxy, phenyl, benzyl, phenylethyl, halogenated phenyl, halogenated benzyl and halogenated phenylethyl.

4. A compound as claimed in claim 1, wherein
R¹ is hydrogen or C₁₋₄alkyl; and R² is selected from cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl wherein said cyclopentyl, 10,11-dihydro-5H-benzo[4,5]cyclohepta[1,2-b]pyridine-5-yl, phenyl, tetrahydronaphthalenylmethyl, 1,7-diazabicyclo[4.3.0]nona-2,4,6,8-teraen-8-ylmethyl, 6,7-diazabicylco[3.3.0]octa-7,9-dien-8-ylmethyl, oxadiazolylmethyl, oxazolylmethyl, 2,3-dihydro-1H-indenyl, 1,2,3,4-tetrahydroquinolinylmethyl, quinolinylmethyl, isoquinolinylmethyl, pyrrolidinyl, pyrazolylmethyl, dihydrobenzofuranylmethyl, pyridinylmethyl, chromanylmethyl, isochromanylmethyl, indolylmethyl, benzothiozolylmethyl, benzyl and phenylethyl are optionally substituted with one or more groups selected from hydroxyl, methoxy, ethoxy, methoxymethyl, cyclopropylmethoxy, halogenated C₁₋₃alkoxy, halogenated C₁₋₃alkyl, halogen, methyl, ethyl, isopropyl, phenyl, benzyl, phenylethyl, halogenated phenyl, halogenated benzyl and halogenated phenylethyl.

5. A compound as claimed in claim 1, wherein R¹ and R² together with the nitrogen connected thereto form a C₃₋₆heterocycloalkyl, wherein said C₃₋₆heterocycloalkyl is optionally substituted with one or more groups selected from halogen, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸—(CH₂)ₘ—C(═O)—OR⁷—(CH₂)ₘ—C(═O)R⁷—(CH₂)ₘ—S(═O)₂R⁷—(CH₂)ₘ—S(═O)R⁷, —(CH₂)ₘ—SR⁷—(CH₂)ₘ—O—C(═O)—R⁷—(CH₂)ₘ—R⁷, (CH₂)ₘ—NR⁷R⁸ hydroxy C₂₋₉heterocyclyl, phenyl, benzyl, phenylethyl, halogenated phenyl, halogenated benzyl and halogenated phenylethyl, wherein said C₂₋₉heterocyclyl, phenyl, benzyl, phenylethyl, halogenated phenyl, halogenated benzyl and halogenated phenylethyl are optionally substituted with one or more groups selected from hydroxyl, methoxy, ethoxy, methoxymethyl, cyclopropylmethoxy, halogen, methyl, ethyl, phenyl, benzyl, phenylethyl, halogenated phenyl, halogenated benzyl and halogenated phenylethyl.

6. A compound as claimed in claim 1, wherein R¹ and R² together with the nitrogen connected thereto form pyrrolidinyl, morpholinyl or azetidinyl, wherein said pyrrolidinyl, morpholinyl, and azetidinyl are optionally substituted with one or more groups selected from methoxy, ethoxy, halogen, methyl, ethyl, quinolinyl, isoquinolinyl, oxadiazolyl, phenyl, and benzyl; wherein said quinolinyl, isoquinolinyl, oxadiazolyl, phenyl, and benzyl groups may be optionally substituted from halogen, cyano, nitro, C₁₋₄alkoxy, C₁₋₆alkyl, halogenated C₁₋₆alkyl, C₃₋₆cycloalkyl, C₃₋₆cycloalkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸—(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷—(CH₂)ₘ—SR⁷, —(CH₂)ₘ—O—C(═O)—R⁷, —(CH₂)ₘ—OR⁷—(CH₂)ₘ—NR⁷R⁸ hydroxy, phenyl, benzyl, phenylethyl, halogenated phenyl, halogenated benzyl and halogenated phenylethyl.

7. A compound as claimed in claim 1, wherein R³ is hydrogen and R⁴ is quinuclidinyl or C₁₋₄alkyl, wherein said quinuclidinyl and C₁₋₄alkyl are optionally substituted with one or more groups selected from methylsulfonyl, dimethylamino, methylamino, acetylamino, hydroxy, methoxy, ethoxy, halogen, methyl, ethyl, 2-oxopyrrolidin-1-yl, tetrahydrofuranyl, phenyl, halogenated phenyl, pyridyl, halogenated pyridyl, halogenated benzyl and benzyl.

8. A compound as claimed in claim 1, wherein R³ and R⁴ together with the nitrogen connected thereto form a C₂₋₉heterocyclyl, wherein said C₂₋₉heterocyclyl is optionally substituted by one or more groups selected from C₁₋₆alkyl, halogenated C₁₋₆alkyl, halogen, methoxy, ethoxy, morpholinyl, hydroxy, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘ—N(R⁷)C(═O)R⁸, —(CH₂)ₘ—C(═O)R⁷, —(CH₂)ₘ—S(═O)₂R⁷, and —(CH₂)ₘ—NR⁷R⁸; wherein R⁷ and R³ are independently selected from —H, C₁₋₆alkyl, and C₃₋₆cycloalkyl-C₀₋₄alkyl; and m is 0, 1, 2 or 3.

9. A compound as claimed in claim 1, wherein R³ and R⁴ together with the nitrogen connected thereto form a group selected from piperazinyl, piperidinyl, hexahydro-oxazolo[3,4-a]pyrazin-3-one-7-yl, hexahydro-pyrrolo[1,2-a]pyrazin-2-yl, 3-oxopiperazin-1-yl, 1,4-diazepan-1-yl, 2,5-diazabicyclo[2.2.1]hept-2-yl, 6-oxohexahydropyrrolo[1,2-a]pyrazin-2(1H)-yl, 5,6-dihydro-[1,2,4]triazolo[4,3-a]pyrazin-7(8H)-yl, morpholinyl and pyrrolidinyl, wherein piperazinyl, piperidinyl, hexahydro-oxazolo[3,4-a]pyrazin-3-one-7-yl, hexahydro-pyrrolo[1,2-a]pyrazin-2-yl, 3-oxopiperazin-1-yl, 1,4-diazepan-1-yl, 2,5-diazabicyclo[2.2.1]hept-2-yl, 6-oxohexahydropyrrolo[1,2-a]pyrazin-2(1H)-yl, 5,6-dihydro-[1,2,4]triazolo[4,3-a]pyrazin-7(8H)-yl, morpholinyl and pyrrolidinyl are optionally substituted by one or more groups selected from methyl, ethyl, isopropyl, cyclopropyl, acetyl, cyclopropylcarbonyl, isopropylcarbonyl, ethylcarbonyl, acetylamino, methylsulfonyl, and dimethylaminocarbonylmethyl.

10. A compound as claimed in claim 1, wherein R⁵ is n-propyl or isopropyl.

11. A compound as claimed in claim 1 wherein each R⁷ and R⁸ are independently selected from —H and C₁₋₆alkyl.

12. A compound as claimed in claim 1 wherein
each R⁷ and R⁸ are independently selected from —H, C₁₋₆alkyl, C₆₋₁₀aryl, C₁₋₅heterocyclyl, and C₃₋₆cycloalkyl-C₀₋₄alkyl, wherein said C₁₋₆alkyl, C₆₋₁₀aryl, C₁₋₅heterocyclyl, and C₃₋₆cycloalkyl-C₀₋₄alkyl are optionally substituted with one or more groups selected from —OH, methoxy, ethoxy and halogen.

13. A compound as claimed in claim 1 wherein m is 0.

14. A compound as claimed in claim 1 wherein m is 1.

15. A compound as claimed in claim 1 wherein m is 2.

16. A compound selected from:
2-(4-acetylpiperazin-1-yl)-6-isopropyl-4-(1,2,3,4-tetrahydronaphthalen-1-ylamino)-5,6-dihydro-7H-pyrrolo[3,4-d]pyrimidin-7-one;
ethyl 3-{[2-(4-acetylpiperazin-1-yl)-6-isopropyl-7-oxo-6,7-dihydro-5H-pyrrolo[3,4-d]pyrimidin-4-yl]amino}-2-phenylpropanoate;
2-(4-acetylpiperazin-1-yl)-4-[benzyl(tetrahydrofuran-2-ylmethyl)amino]-6-isopropyl-5,6-dihydro-7H-pyrrolo[3,4-d]pyrimidin-7-one;
2-(4-acetylpiperazin-1-yl)-4-[cyclopentyl(4-fluorobenzyl)amino]-6-isopropyl-5,6-dihydro-7H-pyrrolo[3,4-d]pyrimidin-7-one;
2-(4-acetylpiperazin-1-yl)-4-{[1-(4-tert-butylphenyl)ethyl]amino}-6-isopropyl-5,6-dihydro-7H-pyrrolo[3,4-d]pyrimidin-7-one;
2-(4-acetylpiperazin-1-yl)-4-{[1-(4-isobutylphenyl)ethyl]amino}-6-isopropyl-5,6-dihydro-7H-pyrrolo[3,4-d]pyrimidin-7-one;
2-{[2-(dimethylamino)ethyl]amino}-6-isopropyl-4-{[(4-methylphenyl)(phenyl)methyl]amino}-5,6-dihydro-7H-pyrrolo[3,4-d]pyrimidin-7-one;
2-(4-acetylpiperazin-1-yl)-4-{[2-(4-chlorophenyl)propyl]amino}-6-isopropyl-5,6-dihydro-7H-pyrrolo[3,4-d]pyrimidin-7-one;
4-{[(4-chlorophenyl)(phenyl)methyl]amino}-2-{[2-(dimethylamino)ethyl]amino}-6-isopropyl-5,6-dihydro-7H-pyrrolo[3,4-d]pyrimidin-7-one;
2-(4-acetylpiperazin-1-yl)-6-isopropyl-4-{[1-(4-isopropylphenyl)-2-methylpropyl]amino}-5,6-dihydro-7H-pyrrolo[3,4-d]pyrimidin-7-one;
2-(4-acetylpiperazin-1-yl)-6-isopropyl-4-{[(1R)-1-(4-methoxyphenyl)ethyl]amino}-5,6-dihydro-7H-pyrrolo[3,4-d]pyrimidin-7-one;
2-(4-acetylpiperazin-1-yl)-4-{[2-(4-fluorophenyl)-1-methylethyl]amino}-6-isopropyl-5,6-dihydro-7H-pyrrolo[3,4-d]pyrimidin-7-one;
2-(4-acetylpiperazin-1-yl)-6-isopropyl-4-{[1-(3-phenyl-1,2,4-oxadiazol-5-yl)ethyl]amino}-5,6-dihydro-7H-pyrrolo[3,4-d]pyrimidin-7-one;
2-(4-acetylpiperazin-1-yl)-4-{[2-(4-fluorophenyl)-1,1-dimethylethyl]amino}-6-isopropyl-5,6-dihydro-7H-pyrrolo[3,4-d]pyrimidin-7-one;
2-(4-acetylpiperazin-1-yl)-4-({[1-(4-chlorophenyl)cyclobutyl]methyl}amino)-6-isopropyl-5,6-dihydro-7H-pyrrolo[3,4-d]pyrimidin-7-one;
2-(4-acetylpiperazin-1-yl)-4-{[2-(4-chlorophenyl)-2-methylpropyl]amino}-6-isopropyl-5,6-dihydro-7H-pyrrolo[3,4-d]pyrimidin-7-one;
4-{[bis(4-fluorophenyl)methyl]amino}-2-{[2-(dimethylamino)ethyl]amino}-6-isopropyl-5,6-dihydro-7H-pyrrolo[3,4-d]pyrimidin-7-one;
4-{[(4-chlorophenyl)(phenyl)methyl]amino}-2-(4-ethylpiperazin-1-yl)-6-isopropyl-5,6-dihydro-7H-pyrrolo[3,4-d]pyrimidin-7-one;
4-{[(4-chlorophenyl)(phenyl)methyl]amino}-2-[[2-(dimethylamino)ethyl](methyl)amino]-6-isopropyl-5,6-dihydro-7H-pyrrolo[3,4-d]pyrimidin-7-one;
4-{[(4-chlorophenyl)(phenyl)methyl]amino}-6-isopropyl-2-(5-methyl-2,5-diazabicyclo[2.2.1]hept-2-yl)-5,6-dihydro-7H-pyrrolo[3,4-d]pyrimidin-7-one;
2-{[2-(dimethylamino)ethyl]amino}-4-[(9-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one;
2-{[2-(dimethylamino)ethyl]amino}-4-[(7-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one;
4-{[bis(4-fluorophenyl)methyl]amino}-6-isopropyl-2-(5-methyl-2,5-diazabicyclo[2.2.1]hept-2-yl)-5,6-dihydro-7H-pyrrolo[3,4-d]pyrimidin-7-one;
4-{[bis(4-fluorophenyl)methyl]amino}-2-[[2-(dimethylamino)ethyl](methyl)amino]-6-isopropyl-5,6-dihydro-7H-pyrrolo[3,4-d]pyrimidin-7-one;
2-(4-acetylpiperazin-1-yl)-6-isopropyl-4-({1-[4-(trifluoromethoxy)phenyl]ethyl}amino)-5,6-dihydro-7H-pyrrolo[3,4-d]pyrimidin-7-one;
2-(4-acetylpiperazin-1-yl)-4-{[1-(4-ethoxyphenyl)ethyl]amino}-6-isopropyl-5,6-dihydro-7H-pyrrolo[3,4-d]pyrimidin-7-one;
2-(4-acetylpiperazin-1-yl)-4-{[(4-chlorophenyl)(cyclopropyl)methyl]amino}-6-isopropyl-5,6-dihydro-7H-pyrrolo[3,4-d]pyrimidin-7-one;
2-(4-acetylpiperazin-1-yl)-6-isopropyl-4-({1-[4-(trifluoromethyl)phenyl]ethyl}amino)-5,6-dihydro-7H-pyrrolo[3,4-d]pyrimidin-7-one;
2-(4-acetylpiperazin-1-yl)-6-isopropyl-4-{[1-(4-propylphenyl)ethyl]amino}-5,6-dihydro-7H-pyrrolo[3,4-d]pyrimidin-7-one;
2-(4-acetylpiperazin-1-yl)-6-isopropyl-4-{[4-(trifluoromethoxy)benzyl]amino}-5,6-dihydro-7H-pyrrolo[3,4-d]pyrimidin-7-one;
2-(4-acetylpiperazin-1-yl)-6-isopropyl-4-({1-[4-(trifluoromethyl)phenyl]propyl}amino)-5,6-dihydro-7H-pyrrolo[3,4-d]pyrimidin-7-one;
2-(4-acetylpiperazin-1-yl)-4-{[trans-2-(4-chlorophenyl)cyclopentyl]amino}-6-isopropyl-5,6-dihydro-7H-pyrrolo[3,4-d]pyrimidin-7-one;
2-(4-acetylpiperazin-1-yl)-6-isopropyl-4-{[(1R)-1-(4-methylphenyl)ethyl]amino}-5,6-dihydro-7H-pyrrolo[3,4-d]pyrimidin-7-one;
{[2-(4-acetylpiperazin-1-yl)-6-isopropyl-7-oxo-6,7-dihydro-5H-pyrrolo[3,4-d]pyrimidin-4-yl]amino}[4-(trifluoromethyl)phenyl]acetic acid;
2-(4-acetylpiperazin-1-yl)-6-isopropyl-4-{[1-(4-methylphenyl)propyl]amino}-5,6-dihydro-7H-pyrrolo[3,4-d]pyrimidin-7-one;
2-(4-acetylpiperazin-1-yl)-6-isopropyl-4-{[(1-methyl-1,2,3,4-tetrahydroquinolin-6-yl)methyl]amino}-5,6-dihydro-7H-pyrrolo[3,4-d]pyrimidin-7-one;
2-(4-acetylpiperazin-1-yl)-4-[2-ethyl-2-(4-methylphenyl)hydrazino]-6-isopropyl-5,6-dihydro-7H-pyrrolo[3,4-d]pyrimidin-7-one;
2-(4-acetylpiperazin-1-yl)-6-isopropyl-4-{[1-(4-isopropylphenyl)ethyl]amino}-5,6-dihydro-7H-pyrrolo[3,4-d]pyrimidin-7-one;
2-(4-acetylpiperazin-1-yl)-4-{[2-(4-chlorophenyl)ethyl]amino}-6-isopropyl-5,6-dihydro-7H-pyrrolo[3,4-d]pyrimidin-7-one;
2-(4-acetylpiperazin-1-yl)-6-isopropyl-4-{[2-(4-methylphenyl)ethyl]amino}-5,6-dihydro-7H-pyrrolo[3,4-d]pyrimidin-7-one;
2-(4-acetylpiperazin-1-yl)-6-isopropyl-4-[(4-isopropylbenzyl)amino]-5,6-dihydro-7H-pyrrolo[3,4-d]pyrimidin-7-one;
4-[2-(4-chlorobenzyl)pyrrolidin-1-yl]-6-isopropyl-2-[(2-methoxyethyl)amino]-5,6-dihydro-7H-pyrrolo[3,4-d]pyrimidin-7-one;
N-[2-({4-[2-(4-chlorobenzyl)pyrrolidin-1-yl]-6-isopropyl-7-oxo-6,7-dihydro-5H-pyrrolo[3,4-d]pyrimidin-2-yl}amino)ethyl]acetamide;
2-(4-acetylpiperazin-1-yl)-4-[2-(2-chlorobenzyl)pyrrolidin-1-yl]-6-isopropyl-5,6-dihydro-7H-pyrrolo[3,4-d]pyrimidin-7-one;
2-(4-acetylpiperazin-1-yl)-6-isopropyl-4-[2-(4-methylbenzyl)pyrrolidin-1-yl]-5,6-dihydro-7H-pyrrolo[3,4-d]pyrimidin-7-one;
2-(4-acetylpiperazin-1-yl)-4-[2-(3-chlorobenzyl)pyrrolidin-1-yl]-6-isopropyl-5,6-dihydro-7H-pyrrolo[3,4-d]pyrimidin-7-one;
4-[2-(4-chlorobenzyl)pyrrolidin-1-yl]-6-isopropyl-2-(3-oxopiperazin-1-yl)-5,6-dihydro-7H-pyrrolo[3,4-d]pyrimidin-7-one;
2-(4-acetylpiperazin-1-yl)-4-[2-(4-chlorobenzyl)pyrrolidin-1-yl]-6-isopropyl-5,6-dihydro-7H-pyrrolo[3,4-d]pyrimidin-7-one;
2-(4-acetylpiperazin-1-yl)-4-[benzyl(cyclopropylmethyl)amino]-6-isopropyl-5,6-dihydro-7H-pyrrolo[3,4-d]pyrimidin-7-one;
2-(4-acetylpiperazin-1-yl)-4-[2-(4-chlorophenyl)pyrrolidin-1-yl]-6-isopropyl-5,6-dihydro-7H-pyrrolo[3,4-d]pyrimidin-7-one;
2-(4-acetylpiperazin-1-yl)-4-{[1-(4-chlorophenyl)ethyl]amino}-6-isopropyl-5,6-dihydro-7H-pyrrolo[3,4-d]pyrimidin-7-one;
2-(4-acetylpiperazin-1-yl)-4-(2-benzylpyrrolidin-1-yl)-6-isopropyl-5,6-dihydro-7H-pyrrolo[3,4-d]pyrimidin-7-one;
2-(4-acetylpiperazin-1-yl)-4-{[2-(3,4-dimethylphenyl)ethyl]amino}-6-isopropyl-5,6-dihydro-7H-pyrrolo[3,4-d]pyrimidin-7-one;
2-(4-acetylpiperazin-1-yl)-4-{[2-(2,4-dimethylphenyl)ethyl]amino}-6-isopropyl-5,6-dihydro-7H-pyrrolo[3,4-d]pyrimidin-7-one;
4-[2-(4-chlorobenzyl)pyrrolidin-1-yl]-2-{[2-(dimethylamino)ethyl]amino}-6-isopropyl-5,6-dihydro-7H-pyrrolo[3,4-d]pyrimidin-7-one;
2-(4-acetylpiperazin-1-yl)-4-[benzyl(4-chlorobenzyl)amino]-6-isopropyl-5,6-dihydro-7H-pyrrolo[3,4-d]pyrimidin-7-one;
2-(4-acetylpiperazin-1-yl)-4-[(4-chlorobenzyl)(cyclopropylmethyl)amino]-6-isopropyl-5,6-dihydro-7H-pyrrolo[3,4-d]pyrimidin-7-one;
2-(4-acetylpiperazin-1-yl)-4-{[2-(4-chlorophenyl)-1,1-dimethylethyl]amino}-6-isopropyl-5,6-dihydro-7H-pyrrolo[3,4-d]pyrimidin-7-one;
4-{[2-(4-chlorophenyl)-1,1-dimethylethyl]amino}-2-[(2R,6S)-2,6-dimethylmorpholin-4-yl]-6-isopropyl-5,6-dihydro-7H-pyrrolo[3,4-d]pyrimidin-7-one;
N-[1-(4-{[2-(4-chlorophenyl)-1,1-dimethylethyl]amino}-6-isopropyl-7-oxo-6,7-dihydro-5H-pyrrolo[3,4-d]pyrimidin-2-yl)pyrrolidin-3-yl]acetamide;
2-[4-(4-{[2-(4-chlorophenyl)-1,1-dimethylethyl]amino}-6-isopropyl-7-oxo-6,7-dihydro-5H-pyrrolo[3,4-d]pyrimidin-2-yl)piperazin-1-yl]-N,N-dimethylacetamide;
4-{[2-(4-chlorophenyl)-1,1-dimethylethyl]amino}-2-(4-ethylpiperazin-1-yl)-6-isopropyl-5,6-dihydro-7H-pyrrolo[3,4-d]pyrimidin-7-one;
2-(4-acetyl-1,4-diazepan-1-yl)-4-{[2-(4-chlorophenyl)-1,1-dimethylethyl]amino}-6-isopropyl-5,6-dihydro-7H-pyrrolo[3,4-d]pyrimidin-7-one;
4-{[2-(4-chlorophenyl)-1,1-dimethylethyl]amino}-6-isopropyl-2-{[3-(2-oxopyrrolidin-1-yl)propyl]amino}-5,6-dihydro-7H-pyrrolo[3,4-d]pyrimidin-7-one;
N-[1-(4-{[2-(4-chlorophenyl)-1,1-dimethylethyl]amino}-6-isopropyl-7-oxo-6,7-dihydro-5H-pyrrolo[3,4-d]pyrimidin-2-yl)pyrrolidin-3-yl]-N-methylacetamide;
4-{[2-(4-chlorophenyl)-1,1-dimethylethyl]amino}-6-isopropyl-2-(5-oxo-1,4-diazepan-1-yl)-5,6-dihydro-7H-pyrrolo[3,4-d]pyrimidin-7-one;
4-{[2-(4-chlorophenyl)-1,1-dimethylethyl]amino}-6-isopropyl-2-(4-methyl-3-oxopiperazin-1-yl)-5,6-dihydro-7H-pyrrolo[3,4-d]pyrimidin-7-one;
4-{[2-(4-chlorophenyl)-1,1-dimethylethyl]amino}-6-isopropyl-2-{[2-(2-oxopyrrolidin-1-yl)ethyl]amino}-5,6-dihydro-7H-pyrrolo[3,4-d]pyrimidin-7-one;
4-{[2-(4-chlorophenyl)-1,1-dimethylethyl]amino}-6-isopropyl-2-(4-propionylpiperazin-1-yl)-5,6-dihydro-7H-pyrrolo[3,4-d]pyrimidin-7-one;
2-(4-Acetyl-piperazin-1-yl)-4-(benzhydryl-amino)-6-isopropyl-5,6-dihydro-pyrrolo[3,4-d]pyrimidin-7-one;
4-(Benzhydryl-amino)-6-isobutyl-2-morpholin-4-yl-5,6-dihydro-pyrrolo[3,4-d]pyrimidin-7-one;
4-(Benzhydryl-amino)-6-cyclopropyl-2-morpholin-4-yl-5,6-dihydro-pyrrolo[3,4-d]pyrimidin-7-one;
4-(Benzhydryl-amino)-6-cyclopentyl-2-morpholin-4-yl-5,6-dihydro-pyrrolo[3,4-d]pyrimidin-7-one;
6-isopropyl-2-morpholin-4-yl-[(phenyl-p-tolyl-methyl)-amino]-5,6-dihydro-pyrrolo[3,4-d]pyrimidin-7-one;
4-{[(4-Chloro-phenyl)-phenyl-methyl]-amino}-6-isopropyl-2-morpholin-4-yl-5,6-dihydro-pyrrolo[3,4-d]pyrimidin-7-one;
4-(Benzhydryl-amino)-2-(4-ethyl-piperazin-1-yl)-6-isopropyl-5,6-dihydro-pyrrolo[3,4-d]pyrimidin-7-one;
4-(Benzhydryl-amino)-2-(4-cyclopropanecarbonyl-piperazin-1-yl)-6-isopropyl-5,6-dihydro-pyrrolo[3,4-d]pyrimidin-7-one;
4-(Benzhydryl-amino)-2-(4-isobutyryl-piperazin-1-yl)-6-isopropyl-5,6-dihydro-pyrrolo[3,4-d]pyrimidin-7-one;
4-(Benzhydryl-amino)-6-isopropyl-2-morpholin-4-yl-5,6-dihydro-pyrrolo[3,4-d]pyrimidin-7-one;
4-(Benzhydryl-amino)-6-dimethylamino-2-(4-methyl-piperazin-1-yl)-5,6-dihydro-cyclopentapyrimidin-7-one;
4-(Benzhydryl-amino)-6-isopropyl-2-piperazin-1-yl-5,6-dihydro-pyrrolo[3,4-d]pyrimidin-7-one;
4-(Benzhydryl-amino)-2-benzylamino-6-isopropyl-5,6-dihydro-pyrrolo[3,4-d]pyrimidin-7-one;
4-(Benzhydryl-amino)-6-isopropyl-2-(2-methoxy-ethylamino)-5,6-dihydro-pyrrolo[3,4-d]pyrimidin-7-one;
4-(Benzhydryl-amino)-2-(2,6-dimethyl-morpholin-4-yl)-6-isopropyl-5,6-dihydro-pyrrolo[3,4-d]pyrimidin-7-one;
4-(Benzhydryl-amino)-6-isopropyl-2-(4-propionyl-piperazin-1-yl)-5,6-dihydro-pyrrolo[3,4-d]pyrimidin-7-one;
2-(4-Acetyl-piperazin-1-yl)-4-(1,2-diphenyl-ethylamino)-6-isopropyl-5,6-dihydro-pyrrolo[3,4-d]pyrimidin-7-one;
4-(Benzhydryl-amino)-2-diethylamino-6-isopropyl-5,6-dihydro-pyrrolo[3,4-d]pyrimidin-7-one;
2-{4-[4-(Benzhydryl-amino)-6-isopropyl-7-oxo-6,7-dihydro-5H-pyrrolo[3,4-d]pyrimidin-2-yl]-piperazin-1-yl}-N,N-dimethyl-acetamide;
2-(4-acetylpiperazin-1-yl)-4-[(2,2-diphenylethyl)amino]-6-isopropyl-5,6-dihydro-7H-pyrrolo[3,4-d]pyrimidin-7-one;
4-(Benzhydryl-amino)-6-isopropyl-2-(4-methanesulfonyl-piperazin-1-yl)-5,6-dihydro-pyrrolo[3,4-d]pyrimidin-7-one;
N-{2-[4-(Benzhydryl-amino)-6-isopropyl-7-oxo-6,7-dihydro-5H-pyrrolo[3,4-d]pyrimidin-2-ylamino]-ethyl}-acetamide;
4-(Benzhydryl-amino)-6-isopropyl-2-[(pyridin-3-ylmethyl)-amino]-5,6-dihydro-pyrrolo[3,4-d]pyrimidin-7-one;
2-(4-Acetyl-piperazin-1-yl)-4-dibenzylamino-6-isopropyl-5,6-dihydro-pyrrolo[3,4-d]-pyrimidin-7-one;
N-{1-[4-(Benzhydryl-amino)-6-isopropyl-7-oxo-6,7-dihydro-5H-pyrrolo[3,4-d]pyrimidin-2-yl]-pyrrolidin-3-yl}-acetamide;
4-(Benzhydryl-amino)-2-(2-dimethylamino-ethylamino)-6-isopropyl-5,6-dihydro-pyrrolo[3,4-d]pyrimidin-7-one;
6-isopropyl-2-(2-methoxy-ethylamino)-4-[(phenyl-p-tolyl-methyl)-amino]-5,6-dihydro-pyrrolo[3,4-d]-pyrimidin-7-one;
4-(Benzhydryl-amino)-2-[4-(2-dimethylamino-acetyl)-piperazin-1-yl]-6-isopropyl-5,6-dihydro-pyrrolo[3,4-d]pyrimidin-7-one;
4-(Benzhydryl-amino)-6-dimethylamino-2-(2-hydroxy-ethylamino)-5,6-dihydro-cyclopentapyrimidin-7-one;
2-(4-Ethyl-piperazin-1-yl)-6-isopropyl-4-[(phenyl-p-tolyl-methyl)-amino]-5,6-dihydro-pyrrolo[3,4-d]-pyrimidin-7-one;
2-(4-isopropyl-piperazin-1-yl)-6-isopropyl-4-[(phenyl-p-tolyl-methyl)-amino]-5,6-dihydro-pyrrolo[3,4-d]-pyrimidin-7-one;
2-(4-Acetyl-piperazin-1-yl)-6-isopropyl-4-{[(4-methoxy-phenyl)-phenyl-methyl]-amino}-5,6-dihydro-pyrrolo[3,4-d]pyrimidin-7-one;
2-(4-Acetyl-piperazin-1-yl)-4-(4-chloro-benzylamino)-6-isopropyl-5,6-dihydro-pyrrolo[3,4-d]-pyrimidin-7-one;
2-(4-Acetyl-piperazin-1-yl)-6-isopropyl-4-(3-isopropyl-phenyl amino)-5,6-dihydro-pyrrolo[3,4-d]-pyrimidin-7-one;
4-Dibenzylamino-2-(2-dimethylamino-ethylamino)-6-isopropyl-5,6-dihydro-pyrrolo[3,4-d]-pyrimidin-7-one;
4-(2,2-Diphenyl-ethylamino)-2-(4-ethyl-piperazin-1-yl)-6-isopropyl-5,6-dihydro-pyrrolo[3,4-d]pyrimidin-7-one;
2-(3-Dimethylamino-propylamino)-4-(2,2-diphenyl-ethylamino)-6-isopropyl-5,6-dihydro-pyrrolo[3,4-d]pyrimidin-7-one;
4-(2,2-Diphenyl-ethylamino)-6-isopropyl-2-(2-methylamino-ethylamino)-5,6-dihydro-pyrrolo[3,4-d]pyrimidin-7-one;
4-[(diphenylmethyl)amino]-2-morpholin-4-yl-6-propyl-5,6-dihydro-7H-pyrrolo[3,4-d]pyrimidin-7-one;
2-(4-acetylpiperazin-1-yl)-4-[(4-chlorobenzyl)(methyl)amino]-6-isopropyl-5,6-dihydro-7H-pyrrolo[3,4-d]pyrimidin-7-one;
2-(4-acetylpiperazin-1-yl)-6-isopropyl-4-({(1R)-1-[4-(trifluoromethoxy)phenyl]ethyl}amino)-5,6-dihydro-7H-pyrrolo[3,4-d]pyrimidin-7-one;
2-(4-acetylpiperazin-1-yl)-4-({[1-(4-chlorophenyl)cyclopentyl]methyl}amino)-6-isopropyl-5,6-dihydro-7H-pyrrolo[3,4-d]pyrimidin-7-one;
2-(4-acetylpiperazin-1-yl)-4-({(1R)-1-[4-(difluoromethoxy)phenyl]ethyl}amino)-6-isopropyl-5,6-dihydro-7H-pyrrolo[3,4-d]pyrimidin-7-one;
2-(4-acetylpiperazin-1-yl)-4-{[(1R)-1-(4-ethoxyphenyl)ethyl]amino}-6-isopropyl-5,6-dihydro-7H-pyrrolo[3,4-d]pyrimidin-7-one;
2-(4-acetylpiperazin-1-yl)-4-[(2R)-2-benzylpyrrolidin-1-yl]-6-isopropyl-5,6-dihydro-7H-pyrrolo[3,4-d]pyrimidin-7-one;
2-(4-acetylpiperazin-1-yl)-4-[(2S)-2-benzylpyrrolidin-1-yl]-6-isopropyl-5,6-dihydro-7H-pyrrolo[3,4-d]pyrimidin-7-one;
2-(4-acetylpiperazin-1-yl)-6-ethyl-4-{[2-(4-fluorophenyl)-1,1-dimethylethyl]amino}-5,6-dihydro-7H-pyrrolo[3,4-d]pyrimidin-7-one;
2-(4-ethylpiperazin-1-yl)-6-isopropyl-4-{[phenyl(pyridin-2-yl)methyl]amino}-5,6-dihydro-7H-pyrrolo[3,4-d]pyrimidin-7-one;
2-(4-ethylpiperazin-1-yl)-4-[(9-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one;
2-[[2-(dimethylamino)ethyl](methyl)amino]-4-[(9-fluoro-10,11-dihydro-5H-benzo[4,5]cyclohepta[1,2-b]pyridin-5-yl)amino]-6-isopropyl-5,6-dihydro-7H-pyrrolo[3,4-d]pyrimidin-7-one;
2-(4-acetylpiperazin-1-yl)-6-isopropyl-4-{[2-methyl-4-(trifluoromethoxy)benzyl]amino}-5,6-dihydro-7H-pyrrolo[3,4-d]pyrimidin-7-one;
2-(4-acetylpiperazin-1-yl)-4-[2-(4-chlorophenyl)-2-methylmorpholin-4-yl]-6-isopropyl-5,6-dihydro-7H-pyrrolo[3,4-d]pyrimidin-7-one;
4-{[2-(4-chlorophenyl)-1,1-dimethylethyl]amino}-2-(4-isobutyrylpiperazin-1-yl)-6-isopropyl-5,6-dihydro-7H-pyrrolo[3,4-d]pyrimidin-7-one;
4-{[2-(4-chlorophenyl)-1,1-dimethylethyl]amino}-2-[4-(2,2-dimethylpropanoyl)piperazin-1-yl]-6-isopropyl-5,6-dihydro-7H-pyrrolo[3,4-d]pyrimidin-7-one;
4-{[2-(4-chlorophenyl)-1,1-dimethylethyl]amino}-2-[4-(cyclopropylcarbonyl)piperazin-1-yl]-6-isopropyl-5,6-dihydro-7H-pyrrolo[3,4-d]pyrimidin-7-one;
4-(4-{[2-(4-chlorophenyl)-1,1-dimethylethyl]amino}-6-isopropyl-7-oxo-6,7-dihydro-5H-pyrrolo[3,4-d]pyrimidin-2-yl)-N,N-dimethylpiperazine-1-carboxamide;
4-(4-{[2-(4-chlorophenyl)-1,1-dimethylethyl]amino}-6-isopropyl-7-oxo-6,7-dihydro-5H-pyrrolo[3,4-d]pyrimidin-2-yl)piperazine-1-carbaldehyde;
2-(4-Acetylpiperazin-1-yl)-4-[2-(4-fluorobenzyl)-pyrrolidin-1-yl]-6-isopropyl-5H-pyrrolo[3,4-d]pyrimidin-7(6H)-one;
2-(4-Acetylpiperazin-1-yl)-4-[1-(4-fluorophenyl)-cyclopropylamino]-6-isopropyl-5H-pyrrolo[3,4-d]pyrimidin-7(6H)-one;
2-(4-Acetylpiperazin-1-yl)-6-isopropyl-4-(2-(3-methoxyphenyl)pyrrolidin-1-yl)-5H-pyrrolo[3,4-d]pyrimidin-7(6H)-one;
2-(4-Acetylpiperazin-1-yl)-4-(2-(3-chlorophenyl)pyrrolidin-1-yl)-6-isopropyl-5H-pyrrolo[3,4-d]pyrimidin-7(6H)-one;
4-[(4-benzylphenyl)amino]-6-isopropyl-2-morpholin-4-yl-5,6-dihydro-7H-pyrrolo[3,4-d]pyrimidin-7-one;
2-(4-Acetylpiperazin-1-yl)-4-(5-chloro-2,3-dihydro-1H-inden-1-ylamino)-6-isopropyl-5H-pyrrolo[3,4-d]pyrimidin-7(6H)-one;
4-(1-(4-Fluorophenyl)-2-methylpropan-2-ylamino)-6-isopropyl-2-(6-oxohexahydro[1,2-a]pyrazin-2(1H)-yl)-5H-pyrrolo[3,4-d]pyrimidin-7(6H)-one;
6-Isopropyl-2-(6-oxohexahydropyrrolo[1,2-a]pyrazin-2(1H)-yl)-4-(quinolin-3-ylmethylamino)-5H-pyrrolo[3,4-d]pyrimidin-7(6H)-one;
6-Isopropyl-2-(6-oxohexahydropyrrolo[1,2-a]pyrazin-2(1H)-yl)-4-(quinolin-3-ylmethylamino)-5H-pyrrolo[3,4-d]pyrimidin-7(6H)-one;
2-(5,6-Dihydro-[1,2,4]triazolo[4,3-a]pyrazin-7(8H)-yl)-6-isopropyl-4-(quinolin-3-ylmethylamino)-5H-pyrrolo[3,4-d]pyrimidin-7(6H)-one;
2-(4-Acetyl-3-methyl-piperazin-1-yl)-4-[2-(4-fluoro-phenyl)-1,1-dimethyl-ethylamino]-6-isopropyl-5,6-dihydro-pyrrolo[3,4-d]pyrimidin-7-one;
2-(4-Acetyl-2-methyl-piperazin-1-yl)-4-[2-(4-fluoro-phenyl)-1,1-dimethyl-ethylamino]-6-isopropyl-5,6-dihydro-pyrrolo[3,4-d]pyrimidin-7-one;
(R)-7-{4-[2-(4-Fluoro-phenyl)-1,1-dimethyl-ethylamino]-6-isopropyl-7-oxo-6,7-dihydro-5H-pyrrolo[3,4-d]pyrimidin-2-yl}-hexahydro-oxazolo[3,4-a]pyrazin-3-one;
(R)-2-(Hexahydro-pyrrolo[1,2-a]pyrazin-2-yl)-6-isopropyl-4-[(quinolin-3-ylmethyl)-amino]-5,6-dihydro-pyrrolo[3,4-d]pyrimidin-7-one;
2-(4-Acetyl-3-methyl-piperazin-1-yl)-6-isopropyl-4-[(quinolin-3-ylmethyl)-amino]-5,6-dihydro-pyrrolo[3,4-d]pyrimidin-7-one;
4-(4-acetylpiperazin-1-yl)-8-propan-2-yl-2-[(5-tert-butyl-1H-pyrazol-3-yl)methylamino]-3,5,8-triazabicyclo[4.3.0]nona-1,3,5-trien-7-one;
4-(4-acetylpiperazin-1-yl)-2-[(5-phenyl-1,2-oxazol-3-yl)methylamino]-8-propan-2-yl-3,5,8-triazabicyclo[4.3.0]nona-1,3,5-trien-7-one;
4-(4-acetylpiperazin-1-yl)-2-[(3-phenyl-1,2,4-oxadiazol-5-yl)methylamino]-8-propan-2-yl-3,5,8-triazabicyclo[4.3.0]nona-1,3,5-trien-7-one;
4-(4-acetylpiperazin-1-yl)-2-[(1-phenylpyrazol-4-yl)methylamino]-8-propan-2-yl-3,5,8-triazabicyclo[4.3.0]nona-1,3,5-trien-7-one;
4-(4-acetylpiperazin-1-yl)-2-[(3-phenyl-1,2-oxazol-5-yl)methylamino]-8-propan-2-yl-3,5,8-triazabicyclo[4.3.0]nona-1,3,5-trien-7-one;
4-(4-acetylpiperazin-1-yl)-2-[(5-phenyl-1,3,4-oxadiazol-2-yl)methylamino]-8-propan-2-yl-3,5,8-triazabicyclo[4.3.0]nona-1,3,5-trien-7-one;
4-(4-acetylpiperazin-1-yl)-2-[1-[1-(2-fluorophenyl)pyrazol-4-yl]ethylamino]-8-propan-2-yl-3,5,8-triazabicyclo[4.3.0]nona-1,3,5-trien-7-one;
4-(4-acetylpiperazin-1-yl)-2-[1-(1-phenylpyrazol-4-yl)ethylamino]-8-propan-2-yl-3,5,8-triazabicyclo[4.3.0]nona-1,3,5-trien-7-one;
4-(4-acetylpiperazin-1-yl)-2-(6,7-diazabicyclo[3.3.0]octa-7,9-dien-8-ylmethylamino)-8-propan-2-yl-3,5,8-triazabicyclo[4.3.0]nona-1,3,5-trien-7-one;
4-(4-acetylpiperazin-1-yl)-2-[(1-cyclopentyl-3-methyl-pyrazol-4-yl)methylamino]-8-propan-2-yl-3,5,8-triazabicyclo[4.3.0]nona-2,4,10-trien-7-one;
4-(4-acetylpiperazin-1-yl)-2-[(1-methyl-5-phenyl-pyrazol-3-yl)methylamino]-8-propan-2-yl-3,5,8-triazabicyclo[4.3.0]nona-1,3,5-trien-7-one;
4-(4-acetylpiperazin-1-yl)-2-[(2-methyl-5-phenyl-pyrazol-3-yl)methylamino]-8-propan-2-yl-3,5,8-triazabicyclo[4.3.0]nona-1,3,5-trien-7-one;
4-(4-acetylpiperazin-1-yl)-2-(1,7-diazabicyclo[4.3.0]nona-2,4,6,8-tetraen-8-ylmethylamino)-8-propan-2-yl-3,5,8-triazabicyclo[4.3.0]nona-1,3,5-trien-7-one;
(R)-2-(4-acetylpiperazin-1-yl)-4-(1-(3-ethoxyphenyl)ethylamino)-6-isopropyl-5H-pyrrolo[3,4-d]pyrimidin-7(6H)-one;
2-(4-acetylpiperazin-1-yl)-4-((1-(4-ethoxyphenyl)ethyl)(methyl)amino)-6-isopropyl-5H-pyrrolo[3,4-d]pyrimidin-7(6H)-one;
(R)-2-(4-acetylpiperazin-1-yl)-4-(1-(4-ethoxy-3-fluorophenyl)ethylamino)-6-isopropyl-5H-pyrrolo[3,4-d]pyrimidin-7(6H)-one;
(R)-2-(4-acetylpiperazin-1-yl)-4-(1-(3-chloro-4-ethoxyphenyl)ethylamino)-6-isopropyl-5H-pyrrolo[3,4-d]pyrimidin-7(6H)-one;
(R)-2-(4-acetylpiperazin-1-yl)-4-(1-(2,3-dihydrobenzofuran-5-yl)ethylamino)-6-isopropyl-5H-pyrrolo[3,4-d]pyrimidin-7(6H)-one;
(R)-2-(4-(1-(2-(4-acetylpiperazin-1-yl)-6-isopropyl-7-oxo-6,7-dihydro-5H-pyrrolo[3,4-d]pyrimidin-4-ylamino)ethyl)phenyl)-2-methylpropanenitrile;
2-(4-acetylpiperazin-1-yl)-4-((cyclopropylmethyl)(4-ethoxybenzyl)amino)-6-isopropyl-5H-pyrrolo[3,4-d]pyrimidin-7(6H)-one;
2-(4-(1-(2-(4-acetylpiperazin-1-yl)-6-isopropyl-7-oxo-6,7-dihydro-5H-pyrrolo[3,4-d]pyrimidin-4-ylamino)ethyl)-2-fluorophenyl)-2-methylpropanenitrile;
2-(4-acetylpiperazin-1-yl)-6-isopropyl-4-(2-methyl-1-p-tolylpropan-2-ylamino)-5H-pyrrolo[3,4-d]pyrimidin-7(6H)-one;
(R)-2-(4-acetylpiperazin-1-yl)-4-(1-(4-iodophenyl)ethylamino)-6-isopropyl-5H-pyrrolo[3,4-d]pyrimidin-7(6H)-one;
(R)-2-(4-acetylpiperazin-1-yl)-6-isopropyl-4-(1-(5,6,7,8-tetrahydronaphthalen-2-yl)ethylamino)-5H-pyrrolo[3,4-d]pyrimidin-7(6H)-one;
(R)-2-(4-acetylpiperazin-1-yl)-4-(1-(4-ethoxy-3-methylphenyl)ethylamino)-6-isopropyl-5H-pyrrolo[3,4-d]pyrimidin-7(6H)-one;
(R)-2-(4-acetylpiperazin-1-yl)-4-(1-(4-ethoxy-2-methylphenyl)ethylamino)-6-isopropyl-5H-pyrrolo[3,4-d]pyrimidin-7(6H)-one;
(R)-2-(4-acetylpiperazin-1-yl)-4-(1-(2,2-dimethylchroman-6-yl)ethylamino)-6-isopropyl-5H-pyrrolo[3,4-d]pyrimidin-7(6H)-one;
(R)-2-(4-acetylpiperazin-1-yl)-4-(1-(3,4-dimethylphenyl)ethylamino)-6-isopropyl-5H-pyrrolo[3,4-d]pyrimidin-7(6H)-one;
(R)-2-(4-acetylpiperazin-1-yl)-4-(1-(4-chloro-3-(trifluoromethyl)phenyl)ethylamino)-6-isopropyl-5H-pyrrolo[3,4-d]pyrimidin-7(6H)-one;
(R)-2-(4-acetylpiperazin-1-yl)-4-(1-(2,3-dihydro-1H-inden-5-yl)ethylamino)-6-isopropyl-5H-pyrrolo[3,4-d]pyrimidin-7(6H)-one;
(R)-2-(4-acetylpiperazin-1-yl)-4-(1-(2-ethoxyphenyl)ethylamino)-6-isopropyl-5H-pyrrolo[3,4-d]pyrimidin-7(6H)-one;
(R)-2-(4-acetylpiperazin-1-yl)-4-(1-(3-ethoxy-4-methylphenyl)ethylamino)-6-isopropyl-5H-pyrrolo[3,4-d]pyrimidin-7(6H)-one;
(R)-2-(4-acetylpiperazin-1-yl)-4-(1-(4-ethoxyphenyl)propylamino)-6-isopropyl-5H-pyrrolo[3,4-d]pyrimidin-7(6H)-one;
(R)-2-(4-acetylpiperazin-1-yl)-4-(1-(4-isopropoxyphenyl)ethylamino)-6-isopropyl-5H-pyrrolo[3,4-d]pyrimidin-7(6H)-one;
(S)-2-(4-acetylpiperazin-1-yl)-4-(1-(4-ethoxyphenyl)-2,2,2-trifluoroethylamino)-6-isopropyl-5H-pyrrolo[3,4-d]pyrimidin-7(6H)-one;
2-(4-acetylpiperazin-1-yl)-4-(1-(2,3-dihydrobenzo[b][1,4]dioxin-2-yl)ethylamino)-6-isopropyl-5H-pyrrolo[3,4-d]pyrimidin-7(6H)-one (Isomer 1);
2-(4-acetylpiperazin-1-yl)-4-(1-(2,3-dihydrobenzo[b][1,4]dioxin-2-yl)ethylamino)-6-isopropyl-5H-pyrrolo[3,4-d]pyrimidin-7(6H)-one (Isomer 2);
(R)-2-(4-acetylpiperazin-1-yl)-4-(1-(4-ethoxyphenyl)-2-hydroxyethylamino)-6-isopropyl-5H-pyrrolo[3,4-d]pyrimidin-7(6H)-one;
(R)-2-(4-acetylpiperazin-1-yl)-4-(1-(3-(difluoromethoxy)phenyl)ethylamino)-6-isopropyl-5H-pyrrolo[3,4-d]pyrimidin-7(6H)-one;
(R)-2-(4-acetylpiperazin-1-yl)-4-(1-(3-fluoro-4-(trifluoromethyl)phenyl)ethylamino)-6-isopropyl-5H-pyrrolo[3,4-d]pyrimidin-7(6H)-one;
(R)-2-(4-acetylpiperazin-1-yl)-4-(1-(2-fluoro-5-(trifluoromethyl)phenyl)ethylamino)-6-isopropyl-5H-pyrrolo[3,4-d]pyrimidin-7(6H)-one;
(R)-2-(4-acetylpiperazin-1-yl)-4-(1-(3-fluoro-5-(trifluoromethyl)phenyl)ethylamino)-6-isopropyl-5H-pyrrolo[3,4-d]pyrimidin-7(6H)-one;
(R)-2-(4-acetylpiperazin-1-yl)-4-(cyclopropyl(4-ethoxyphenyl)methylamino)-6-isopropyl-5H-pyrrolo[3,4-d]pyrimidin-7(6H)-one;
(R)-2-(4-acetylpiperazin-1-yl)-4-(1-(2-fluoro-4-(trifluoromethyl)phenyl)ethylamino)-6-isopropyl-5H-pyrrolo[3,4-d]pyrimidin-7(6H)-one;
(R)-2-(4-acetylpiperazin-1-yl)-4-(1-(2-fluoro-3-(trifluoromethyl)phenyl)ethylamino)-6-isopropyl-5H-pyrrolo[3,4-d]pyrimidin-7(6H)-one;
(R)-2-(4-acetylpiperazin-1-yl)-4-(1-(4-fluoro-3-(trifluoromethyl)phenyl)ethylamino)-6-isopropyl-5H-pyrrolo[3,4-d]pyrimidin-7(6H)-one;
(S)-2-(4-acetylpiperazin-1-yl)-4-(1-(4-ethoxyphenyl)-2-hydroxyethylamino)-6-isopropyl-5H-pyrrolo[3,4-d]pyrimidin-7(6H)-one;
(R)-2-(4-acetylpiperazin-1-yl)-4-(1-(3-fluoro-4-isopropoxyphenyl)ethylamino)-6-isopropyl-5H-pyrrolo[3,4-d]pyrimidin-7(6H)-one;
(R)-2-(4-acetylpiperazin-1-yl)-4-(1-(4-cyclobutoxyphenyl)ethylamino)-6-isopropyl-5H-pyrrolo[3,4-d]pyrimidin-7(6H)-one;
(R)-2-(4-acetylpiperazin-1-yl)-4-(1-(4-cyclopropylphenyl)ethylamino)-6-isopropyl-5H-pyrrolo[3,4-d]pyrimidin-7(6H)-one;
(R)-2-(4-acetylpiperazin-1-yl)-4-(1-(3-fluoro-4-propoxyphenyl)ethylamino)-6-isopropyl-5H-pyrrolo[3,4-d]pyrimidin-7(6H)-one;
(R)-2-(4-acetylpiperazin-1-yl)-4-(1-(5-chloro-6-ethoxypyridin-3-yl)ethylamino)-6-isopropyl-5H-pyrrolo[3,4-d]pyrimidin-7(6H)-one;
2-(4-acetylpiperazin-1-yl)-6-isopropyl-4-(1-(2-methoxyphenyl)-2-methylpropan-2-ylamino)-5H-pyrrolo[3,4-d]pyrimidin-7(6H)-one;
2-(4-acetylpiperazin-1-yl)-6-isopropyl-4-(1-(4-methoxyphenyl)-2-methylpropan-2-ylamino)-5H-pyrrolo[3,4-d]pyrimidin-7(6H)-one;
2-(4-acetylpiperazin-1-yl)-6-isopropyl-4-(2-methyl-1-o-tolylpropan-2-ylamino)-5H-pyrrolo[3,4-d]pyrimidin-7(6H)-one;
2-(4-acetylpiperazin-1-yl)-4-(1-(4-ethoxyphenyl)-2-methylpropan-2-ylamino)-6-isopropyl-5H-pyrrolo[3,4-d]pyrimidin-7(6H)-one;
2-(4-acetylpiperazin-1-yl)-4-(benzo[d]thiazol-2-ylmethylamino)-6-isopropyl-5H-pyrrolo[3,4-d]pyrimidin-7(6H)-one;
6-isopropyl-4-((isoquinolin-3-ylmethyl)(methyl)amino)-2-(4-methyl-3-oxopiperazin-1-yl)-5H-pyrrolo[3,4-d]pyrimidin-7(6H)-one;
(R)-4-(1-(4-ethoxy-3-fluorophenyl)ethylamino)-6-isopropyl-2-(4-methyl-3-oxopiperazin-1-yl)-5H-pyrrolo[3,4-d]pyrimidin-7(6H)-one;
(R)-2-(4-acetylpiperazin-1-yl)-4-(1-(4-ethoxy-2-methoxyphenyl)ethylamino)-6-isopropyl-5H-pyrrolo[3,4-d]pyrimidin-7(6H)-one;
(R)-2-(4-acetylpiperazin-1-yl)-4-(1-(4-isopropoxy-2-methoxyphenyl)ethylamino)-6-isopropyl-5H-pyrrolo[3,4-d]pyrimidin-7(6H)-one;
(R)-2-(4-acetylpiperazin-1-yl)-4-(1-(4-ethoxy-2-fluorophenyl)ethylamino)-6-isopropyl-5H-pyrrolo[3,4-d]pyrimidin-7(6H)-one;
2-(4-acetylpiperazin-1-yl)-4-(isochroman-1-ylmethylamino)-6-isopropyl-5H-pyrrolo[3,4-d]pyrimidin-7(6H)-one;
6-isopropyl-2-(3-oxopiperazin-1-yl)-4-(quinolin-3-ylmethylamino)-5H-pyrrolo[3,4-d]pyrimidin-7(6H)-one;
N-(1-(6-isopropyl-7-oxo-4-(quinolin-3-ylmethylamino)-6,7-dihydro-5H-pyrrolo[3,4-d]pyrimidin-2-yl)pyrrolidin-3-yl)acetamide;
N-(1-(6-isopropyl-7-oxo-4-(quinolin-3-ylmethylamino)-6,7-dihydro-5H-pyrrolo[3,4-d]pyrimidin-2-yl)pyrrolidin-3-yl)-N-methylacetamide;
1-(6-isopropyl-7-oxo-4-(quinolin-3-ylmethylamino)-6,7-dihydro-5H-pyrrolo[3,4-d]pyrimidin-2-yl)piperidine-4-carboxamide;
6-isopropyl-2-(4-methyl-3-oxopiperazin-1-yl)-4-(quinolin-3-ylmethylamino)-5H-pyrrolo[3,4-d]pyrimidin-7(6H)-one;
6-isopropyl-2-morpholino-4-(quinolin-3-ylmethylamino)-5H-pyrrolo[3,4-d]pyrimidin-7(6H)-one;
6-isopropyl-2-[2-(morpholinomethyl)pyrrolidin-1-yl]-4-(3-quinolylmethylamino)-5H-pyrrolo[3,4-d]pyrimidin-7-one;
2-(3-dimethylaminopyrrolidin-1-yl)-6-isopropyl-4-(3-quinolylmethylamino)-5H-pyrrolo[3,4-d]pyrimidin-7-one;
6-isopropyl-4-(3-quinolylmethylamino)-2-(quinuclidin-3-ylamino)-5H-pyrrolo[3,4-d]pyrimidin-7-one;
6-isopropyl-2-(2-methylsulfonylethylamino)-4-(3-quinolylmethylamino)-5H-pyrrolo[3,4-d]pyrimidin-7-one;
2-(3,3-difluoropyrrolidin-1-yl)-6-isopropyl-4-(3-quinolylmethylamino)-5H-pyrrolo[3,4-d]pyrimidin-7-one;
6-isopropyl-2-(methyl-(tetrahydrofuran-2-ylmethyl)amino)-4-(3-quinolylmethylamino)-5H-pyrrolo[3,4-d]pyrimidin-7-one;
2-(4-dimethylamino-1-piperidyl)-6-isopropyl-4-(3-quinolylmethylamino)-5H-pyrrolo[3,4-d]pyrimidin-7-one;
6-(1-methylethyl)-4-[(quinolin-3-ylmethyl)amino]-2-[(tetrahydro-2H-pyran-4-ylmethyl)amino]-5,6-dihydro-7H-pyrrolo[3,4-d]pyrimidin-7-one;
2-(dimethylamino)-6-(1-methylethyl)-4-[(quinolin-3-ylmethyl)amino]-5,6-dihydro-7H-pyrrolo[3,4-d]pyrimidin-7-one;
N,N-dimethyl-4-{6-(1-methylethyl)-7-oxo-4-[(quinolin-3-ylmethyl)amino]-6,7-dihydro-5H-pyrrolo[3,4-d]pyrimidin-2-yl}piperazine-1-carboxamide;
2-[4-(cyclopropylcarbonyl)piperazin-1-yl]-6-(1-methylethyl)-4-[(quinolin-3-ylmethyl)amino]-5,6-dihydro-7H-pyrrolo[3,4-d]pyrimidin-7-one;
2-(4-acetylpiperazin-1-yl)-4-(2-(4-ethylpiperazin-1-yl)-4-(trifluoromethyl)benzylamino)-6-isopropyl-5H-pyrrolo[3,4-d]pyrimidin-7(6H)-one;
2-(4-acetylpiperazin-1-yl)-6-isopropyl-4-(1-m-tolylpyrrolidin-3-ylamino)-5H-pyrrolo[3,4-d]pyrimidin-7(6H)-one;
2-(4-acetylpiperazin-1-yl)-6-isopropyl-4-(2-methoxy-4-(trifluoromethyl)benzylamino)-5H-pyrrolo[3,4-d]pyrimidin-7(6H)-one;
(R)-2-(4-acetylpiperazin-1-yl)-6-isopropyl-4-(1-(2-(trifluoromethyl)phenyl)ethylamino)-5H-pyrrolo[3,4-d]pyrimidin-7(6H)-one;
(R)-2-(4-acetylpiperazin-1-yl)-6-isopropyl-4-(1-(3-methoxyphenyl)ethylamino)-5H-pyrrolo[3,4-d]pyrimidin-7(6H)-one;
2-(4-acetylpiperazin-1-yl)-6-isopropyl-4-((6-phenylpyridin-3-yl)methylamino)-5H-pyrrolo[3,4-d]pyrimidin-7(6H)-one;
2-(4-acetylpiperazin-1-yl)-6-isopropyl-4-(isoquinolin-4-ylmethylamino)-5H-pyrrolo[3,4-d]pyrimidin-7(6H)-one;
(R)-2-(4-acetylpiperazin-1-yl)-6-isopropyl-4-(1-(3-(trifluoromethyl)phenyl)ethylamino)-5H-pyrrolo[3,4-d]pyrimidin-7(6H)-one;
2-(4-acetylpiperazin-1-yl)-6-isopropyl-4-(4-(trifluoromethyl)benzylamino)-5H-pyrrolo[3,4-d]pyrimidin-7(6H)-one;
2-(4-acetylpiperazin-1-yl)-6-isopropyl-4-(2-methoxy-4-(trifluoromethoxy)benzylamino)-5H-pyrrolo[3,4-d]pyrimidin-7(6H)-one;
(R)-2-(4-acetylpiperazin-1-yl)-6-isopropyl-4-(1-(quinolin-3-yl)ethylamino)-5H-pyrrolo[3,4-d]pyrimidin-7(6H)-one;
(S)-2-(4-acetylpiperazin-1-yl)-6-isopropyl-4-(1-(quinolin-3-yl)ethylamino)-5H-pyrrolo[3,4-d]pyrimidin-7(6H)-one;
2-(4-acetylpiperazin-1-yl)-6-isopropyl-4-(quinolin-6-ylmethylamino)-5H-pyrrolo[3,4-d]pyrimidin-7(6H)-one;
2-(4-acetylpiperazin-1-yl)-4-(1-(benzo[d]thiazol-2-yl)ethylamino)-6-isopropyl-5H-pyrrolo[3,4-d]pyrimidin-7(6H)-one;
2-(4-acetylpiperazin-1-yl)-6-isopropyl-4-((1-methyl-1H-indol-2-yl)methylamino)-5H-pyrrolo[3,4-d]pyrimidin-7(6H)-one;
2-(4-acetylpiperazin-1-yl)-6-isopropyl-4-(isoquinolin-3-ylmethylamino)-5H-pyrrolo[3,4-d]pyrimidin-7(6H)-one;
2-(4-acetylpiperazin-1-yl)-6-isopropyl-4-(methyl(quinolin-6-ylmethyl)amino)-5H-pyrrolo[3,4-d]pyrimidin-7(6H)-one;
2-(4-acetylpiperazin-1-yl)-6-isopropyl-4-((8-methoxyquinolin-5-yl)methylamino)-5H-pyrrolo[3,4-d]pyrimidin-7(6H)-one;
2-(4-acetylpiperazin-1-yl)-6-isopropyl-4-((2-methylquinolin-3-yl)methylamino)-5H-pyrrolo[3,4-d]pyrimidin-7(6H)-one;
2-(4-acetylpiperazin-1-yl)-6-isopropyl-4-((2-methylquinolin-6-yl)methylamino)-5H-pyrrolo[3,4-d]pyrimidin-7(6H)-one;
(R)-2-(4-acetylpiperazin-1-yl)-6-isopropyl-4-(1-(quinolin-6-yl)ethylamino)-5H-pyrrolo[3,4-d]pyrimidin-7(6H)-one;
(S)-2-(4-acetylpiperazin-1-yl)-6-isopropyl-4-(1-(quinolin-6-yl)ethylamino)-5H-pyrrolo[3,4-d]pyrimidin-7(6H)-one;
2-(4-acetylpiperazin-1-yl)-4-(ethyl(isoquinolin-3-ylmethyl)amino)-6-isopropyl-5H-pyrrolo[3,4-d]pyrimidin-7(6H)-one;
2-(4-acetylpiperazin-1-yl)-4-(ethyl(quinolin-3-ylmethyl)amino)-6-isopropyl-5H-pyrrolo[3,4-d]pyrimidin-7(6H)-one;
2-(4-acetylpiperazin-1-yl)-6-isopropyl-4-((8-methylquinolin-6-yl)methylamino)-5H-pyrrolo[3,4-d]pyrimidin-7(6H)-one;
2-(4-acetylpiperazin-1-yl)-6-isopropyl-4-(quinolin-2-ylmethylamino)-5H-pyrrolo[3,4-d]pyrimidin-7(6H)-one;
2-(4-acetylpiperazin-1-yl)-6-isopropyl-4-(methyl(quinolin-2-ylmethyl)amino)-5H-pyrrolo[3,4-d]pyrimidin-7(6H)-one;
(R)-2-(4-acetylpiperazin-1-yl)-4-(1-(4-(1,3-difluoropropan-2-yloxy)phenyl)ethylamino)-6-isopropyl-5H-pyrrolo[3,4-d]pyrimidin-7(6H)-one;
(S)-2-(4-acetylpiperazin-1-yl)-4-(1-(4-(1,3-difluoropropan-2-yloxy)phenyl)ethylamino)-6-isopropyl-5H-pyrrolo[3,4-d]pyrimidin-7(6H)-one;
2-(4-acetylpiperazin-1-yl)-4-((1-(dimethylamino)isoquinolin-3-yl)methylamino)-6-isopropyl-5H-pyrrolo[3,4-d]pyrimidin-7(6H)-one;
2-(4-acetylpiperazin-1-yl)-4-((cyclopropylmethyl)(isoquinolin-3-ylmethyl)amino)-6-isopropyl-5H-pyrrolo[3,4-d]pyrimidin-7(6H)-one;
2-(4-acetylpiperazin-1-yl)-6-isopropyl-4-(isopropyl(isoquinolin-3-ylmethyl)amino)-5H-pyrrolo[3,4-d]pyrimidin-7(6H)-one;
2-(4-acetylpiperazin-1-yl)-6-isopropyl-4-((isoquinolin-3-ylmethyl)(methyl)amino)-5H-pyrrolo[3,4-d]pyrimidin-7(6H)-one;
2-(4-acetylpiperazin-1-yl)-4-((2,2-difluoroethyl)(isoquinolin-3-ylmethyl)amino)-6-isopropyl-5H-pyrrolo[3,4-d]pyrimidin-7(6H)-one;
2-(4-acetylpiperazin-1-yl)-4-(ethyl(1-(quinolin-6-yl)ethyl)amino)-6-isopropyl-5H-pyrrolo[3,4-d]pyrimidin-7(6H)-one (Enantiomer 1);
2-(4-acetylpiperazin-1-yl)-4-(ethyl(1-(quinolin-6-yl)ethyl)amino)-6-isopropyl-5H-pyrrolo[3,4-d]pyrimidin-7(6H)-one (Enantiomer 2);
2-(4-acetylpiperazin-1-yl)-6-isopropyl-4-((1-methylisoquinolin-3-yl)methylamino)-5H-pyrrolo[3,4-d]pyrimidin-7(6H)-one;
2-(4-acetylpiperazin-1-yl)-6-isopropyl-4-(methyl(1-(quinolin-6-yl)ethyl)amino)-5H-pyrrolo[3,4-d]pyrimidin-7(6H)-one (Enantiomer 1);
2-(4-acetylpiperazin-1-yl)-6-isopropyl-4-(methyl(1-(quinolin-6-yl)ethyl)amino)-5H-pyrrolo[3,4-d]pyrimidin-7(6H)-one (Enantiomer 2);
(R)-2-(4-acetylpiperazin-1-yl)-4-((1-(4-ethoxy-3-fluorophenyl)ethyl)(methyl)amino)-6-isopropyl-5H-pyrrolo[3,4-d]pyrimidin-7(6H)-one;
2-(4-acetylpiperazin-1-yl)-6-isopropyl-4-(methyl((1-methylisoquinolin-3-yl)methyl)amino)-5H-pyrrolo[3,4-d]pyrimidin-7(6H)-one;
2-(4-acetylpiperazin-1-yl)-6-isopropyl-4-((isoquinolin-3-ylmethyl)(2,2,2-trifluoroethyl)amino)-5H-pyrrolo[3,4-d]pyrimidin-7(6H)-one;
benzyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate;
4-(4-acetylpiperazin-1-yl)-2-(2-benzyl-1-piperidyl)-8-propan-2-yl-3,5,8-triazabicyclo[4.3.0]nona-1,3,5-trien-7-one;
4-(4-acetylpiperazin-1-yl)-2-[2-(4-dimethylaminophenyl)pyrrolidin-1-yl]-8-propan-2-yl-3,5,8-triazabicyclo[4.3.0]nona-1,3,5-trien-7-one;
4-(4-acetylpiperazin-1-yl)-2-[2-[(4-fluorophenyl)methyl]-1-piperidyl]-8-propan-2-yl-3,5,8-triazabicyclo[4.3.0]nona-1,3,5-trien-7-one;
4-(4-acetylpiperazin-1-yl)-2-[2-(3-methylphenyl)pyrrolidin-1-yl]-8-propan-2-yl-3,5,8-triazabicyclo[4.3.0]nona-2,4,10-trien-7-one;
4-(4-acetylpiperazin-1-yl)-2-[2-(3,5-dimethylphenyl)pyrrolidin-1-yl]-8-propan-2-yl-3,5,8-triazabicyclo[4.3.0]nona-2,4,10-trien-7-one;
4-(4-acetylpiperazin-1-yl)-2-(2-phenethylpyrrolidin-1-yl)-8-propan-2-yl-3,5,8-triazabicyclo[4.3.0]nona-1,3,5-trien-7-one;
4-(4-acetylpiperazin-1-yl)-2-[2-(4-ethoxyphenyl)pyrrolidin-1-yl]-8-propan-2-yl-3,5,8-triazabicyclo[4.3.0]nona-2,4,10-trien-7-one;
4-(4-acetylpiperazin-1-yl)-2-(2-benzylazetidin-1-yl)-8-propan-2-yl-3,5,8-triazabicyclo[4.3.0]nona-1,3,5-trien-7-one;
4-(4-acetylpiperazin-1-yl)-8-propan-2-yl-2-(2-quinolin-3-ylpyrrolidin-1-yl)-3,5,8-triazabicyclo[4.3.0]nona-1,3,5-trien-7-one;
4-(4-acetylpiperazin-1-yl)-2-[2-(1-phenylpropyl)pyrrolidin-1-yl]-8-propan-2-yl-3,5,8-triazabicyclo[4.3.0]nona-2,4,10-trien-7-one;
4-(4-acetylpiperazin-1-yl)-2-[(3-cyclopentyl-1,2,4-oxadiazol-5-yl)methylamino]-8-propan-2-yl-3,5,8-triazabicyclo[4.3.0]nona-1,3,5-trien-7-one;
tert-butyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate;
4-(4-acetylpiperazin-1-yl)-2-[(5-cyclobutyl-1,2,4-oxadiazol-3-yl)methylamino]-8-propan-2-yl-3,5,8-triazabicyclo[4.3.0]nona-1,3,5-trien-7-one;
4-(4-acetylpiperazin-1-yl)-2-[(2R)-2-(2,3-dihydroindole-1-carbonyl)pyrrolidin-1-yl]-8-propan-2-yl-3,5,8-triazabicyclo[4.3.0]nona-1,3,5-trien-7-one;
4-(4-acetylpiperazin-1-yl)-2-[2-(3-phenyl-1,2,4-oxadiazol-5-yl)pyrrolidin-1-yl]-8-propan-2-yl-3,5,8-triazabicyclo[4.3.0]nona-1,3,5-trien-7-one;
4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one;
4-(4-acetylpiperazin-1-yl)-8-propan-2-yl-2-(2-quinolin-3-ylpyrrolidin-1-yl)-3,5,8-triazabicyclo[4.3.0]nona-1,3,5-trien-7-one (Enantiomer 1);
4-(4-acetylpiperazin-1-yl)-8-propan-2-yl-2-(2-quinolin-3-ylpyrrolidin-1-yl)-3,5,8-triazabicyclo[4.3.0]nona-1,3,5-trien-7-one (Enantiomer 2);
4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one (Enantiomer 1);
4-(4-acetylpiperazin-1-yl)-8-propan-2-yl-2-(2-quinolin-6-ylpyrrolidin-1-yl)-3,5,8-triazabicyclo[4.3.0]nona-1,3,5-trien-7-one (Enantiomer 2);
2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one;
2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one (Enantiomer 1);
2-(4-acetylpiperazin-1-yl)-6-isopropyl-4-[2-[(4-methoxyphenyl)methyl]pyrrolidin-1-yl]-5H-pyrrolo[3,4-d]pyrimidin-7-one (Enantiomer 2);
2-(4-acetylpiperazin-1-yl)-4-[2-[(4-ethoxyphenyl)methyl]pyrrolidin-1-yl]-6-isopropyl-5H-pyrrolo[4,3-e]pyrimidin-7-one;
2-(4-acetylpiperazin-1-yl)-6-isopropyl-4-[2-[(2-methoxyphenyl)methyl]pyrrolidin-1-yl]-5H-pyrrolo[3,4-d]pyrimidin-7-one;
2-(4-acetylpiperazin-1-yl)-6-isopropyl-4-[2-[(3-methoxyphenyl)methyl]pyrrolidin-1-yl]-5H-pyrrolo[3,4-d]pyrimidin-7-one;
2-(4-acetylpiperazin-1-yl)-4-[2-(4-fluorophenyl)pyrrolidin-1-yl]-6-(1-methylethyl)-5,6-dihydro-7H-pyrrolo[3,4-d]pyrimidin-7-one;
2-(4-acetylpiperazin-1-yl)-4-{2-[4-(methoxymethyl)benzyl]pyrrolidin-1-yl}-6-(1-methylethyl)-5,6-dihydro-7H-pyrrolo[3,4-d]pyrimidin-7-one;
4-[2-(4-acetylbenzyl)pyrrolidin-1-yl]-2-(4-acetylpiperazin-1-yl)-6-(1-methylethyl)-5,6-dihydro-7H-pyrrolo[3,4-d]pyrimidin-7-one;
2-(4-acetylpiperazin-1-yl)-4-[2-(4-methoxy-3-methylphenyl)pyrrolidin-1-yl]-6-(1-methylethyl)-5,6-dihydro-7H-pyrrolo[3,4-d]pyrimidin-7-one;
2-(4-acetylpiperazin-1-yl)-6-(1-methylethyl)-4-[2-(4-methylphenyl)pyrrolidin-1-yl]-5,6-dihydro-7H-pyrrolo[3,4-d]pyrimidin-7-one;
2-(4-acetylpiperazin-1-yl)-4-[2-(3,4-dichlorophenyl)pyrrolidin-1-yl]-6-(1-methylethyl)-5,6-dihydro-7H-pyrrolo[3,4-d]pyrimidin-7-one;
2-(4-acetylpiperazin-1-yl)-4-[2-(3,4-dimethylphenyl)pyrrolidin-1-yl]-6-(1-methylethyl)-5,6-dihydro-7H-pyrrolo[3,4-d]pyrimidin-7-one;
2-(4-acetylpiperazin-1-yl)-4-[2-(4-methoxyphenyl)pyrrolidin-1-yl]-6-(1-methylethyl)-5,6-dihydro-7H-pyrrolo[3,4-d]pyrimidin-7-one;
2-(4-acetylpiperazin-1-yl)-4-[(2R)-2-(4-ethoxy-3-fluorophenyl)pyrrolidin-1-yl]-6-(1-methylethyl)-5,6-dihydro-7H-pyrrolo[3,4-d]pyrimidin-7-one;
(4-chlorophenyl)methyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate;
(2R)-1-[4-(4-acetylpiperazin-1-yl)-7-oxo-8-propan-2-yl-3,5,8-triazabicyclo[4.3.0]nona-1,3,5-trien-2-yl]-N-cyclohexyl-pyrrolidine-2-carboxamide;
4-(4-acetylpiperazin-1-yl)-2-[(2R)-2-(4-methylpiperidine-1-carbonyl)pyrrolidin-1-yl]-8-propan-2-yl-3,5,8-triazabicyclo[4.3.0]nona-2,4,10-trien-7-one;
phenyl (2R)-1-[4-(4-acetylpiperazin-1-yl)-7-oxo-8-propan-2-yl-3,5,8-triazabicyclo[4.3.0]nona-1,3,5-trien-2-yl]pyrrolidine-2-carboxylate;
3-(4-acetylpiperazin-1-yl)-5-[[1-(4-fluorophenyl)-2-methyl-propan-2-yl]amino]-8-(oxan-4-yl)-2,4,8-triazabicyclo[4.3.0]nona-1,3,5-trien-9-one;
(+)-4-(4-acetylpiperazin-1-yl)-8-butan-2-yl-2-[(1-phenylpyrazol-4-yl)methylamino]-3,5,8-triazabicyclo[4.3.0]nona-1,3,5-trien-7-one (Enantiomer 1);
(−)-4-(4-acetylpiperazin-1-yl)-8-butan-2-yl-2-[(1-phenylpyrazol-4-yl)methylamino]-3,5,8-triazabicyclo[4.3.0]nona-1,3,5-trien-7-one (Enantiomer 2);
(+)-2-(4-acetylpiperazin-1-yl)-4-(3-quinolylmethylamino)-6-sec-butyl-5H-pyrrolo[3,4-d]pyrimidin-7-one (Enantiomer 1);
(−)-2-(4-acetylpiperazin-1-yl)-4-(3-quinolylmethylamino)-6-sec-butyl-5H-pyrrolo[3,4-d]pyrimidin-7-one (Enantiomer 2);
(+)-2-(4-acetylpiperazin-1-yl)-4-(3-isoquinolylmethylamino)-6-sec-butyl-5H-pyrrolo[3,4-d]pyrimidin-7-one (Enantiomer 1);
(−)-2-(4-acetylpiperazin-1-yl)-4-(3-isoquinolylmethylamino)-6-sec-butyl-5H-pyrrolo[3,4-d]pyrimidin-7-one (Enantiomer 2);
2-(4-acetylpiperazin-1-yl)-6-sec-butyl-4-(1-(4-fluorophenyl)-2-methylpropan-2-ylamino)-5H-pyrrolo[3,4-d]pyrimidin-7(6H)-one;
2-(4-acetylpiperazin-1-yl)-4-(1-(4-fluorophenyl)-2-methylpropan-2-ylamino)-6-(3-methylbutan-2-yl)-5H-pyrrolo[3,4-d]pyrimidin-7(6H)-one;
2-(4-acetylpiperazin-1-yl)-4-(1-(4-fluorophenyl)-2-methylpropan-2-ylamino)-6-(1-methoxypropan-2-yl)-5H-pyrrolo[3,4-d]pyrimidin-7(6H)-one;
2-(4-acetylpiperazin-1-yl)-6-(2-chlorobenzyl)-4-(1-(4-fluorophenyl)-2-methylpropan-2-ylamino)-5H-pyrrolo[3,4-d]pyrimidin-7(6H)-one;
2-(4-acetylpiperazin-1-yl)-6-[(1S)-1-methylpropyl]-4-[(quinolin-3-ylmethyl)amino]-5,6-dihydro-7H-pyrrolo[3,4-d]pyrimidin-7-one;
2-(4-acetylpiperazin-1-yl)-4-[ethyl(isoquinolin-3-ylmethyl)amino]-6-(1-methylpropyl)-5,6-dihydro-7H-pyrrolo[3,4-d]pyrimidin-7-one (Enantiomer 1);
2-(4-acetylpiperazin-1-yl)-4-[ethyl(isoquinolin-3-ylmethyl)amino]-6-(1-methylpropyl)-5,6-dihydro-7H-pyrrolo[3,4-d]pyrimidin-7-one (Enantiomer 2);
2-(4-acetylpiperazin-1-yl)-6-[(1S)-1-methylpropyl]-4-[methyl(quinolin-3-ylmethyl)amino]-5,6-dihydro-7H-pyrrolo[3,4-d]pyrimidin-7-one;
2-(4-acetylpiperazin-1-yl)-4-{[(1R)-1-(4-ethoxy-3-fluorophenyl)ethyl]amino}-6-[(1S)-1-methylpropyl]-5,6-dihydro-7H-pyrrolo[3,4-d]pyrimidin-7-one;
(R)-2-(4-acetylpiperazin-1-yl)-4-(1-(4-ethoxyphenyl)-2,2,2-trifluoroethylamino)-6-isopropyl-5H-pyrrolo[3,4-d]pyrimidin-7(6H)-one;
(R)-2-(4-acetylpiperazin-1-yl)-4-(1-(4-(cyclopropylmethoxy)-3-fluorophenyl)ethylamino)-6-isopropyl-5H-pyrrolo[3,4-d]pyrimidin-7(6H)-one;
2-(4-acetylpiperazin-1-yl)-6-isopropyl-4-((2-methyl-2,3-dihydrobenzofuran-5-yl)methylamino)-5H-pyrrolo[3,4-d]pyrimidin-7(6H)-one;
2-(4-acetylpiperazin-1-yl)-6-isopropyl-4-(1-(2-methyl-2,3-dihydrobenzofuran-5-yl)ethylamino)-5H-pyrrolo[3,4-d]pyrimidin-7(6H)-one;
(S)—N-(2-(4-acetyl-3-methylpiperazin-1-yl)-6-isopropyl-7-oxo-6,7-dihydro-5H-pyrrolo[3,4-d]pyrimidin-4-yl)-N-(isoquinolin-3-ylmethyl)acetamide;
(R)—N-(2-(4-acetyl-3-methylpiperazin-1-yl)-6-isopropyl-7-oxo-6,7-dihydro-5H-pyrrolo[3,4-d]pyrimidin-4-yl)-N-(isoquinolin-3-ylmethyl)acetamide;
(S)-2-(4-acetyl-3-methylpiperazin-1-yl)-6-isopropyl-4-(quinolin-3-ylmethylamino)-5H-pyrrolo[3,4-d]pyrimidin-7(6H)-one;
(R)-2-(4-acetyl-3-methylpiperazin-1-yl)-6-isopropyl-4-(quinolin-3-ylmethylamino)-5H-pyrrolo[3,4-d]pyrimidin-7(6H)-one;
6-(1-methylethyl)-2-[4-(methylsulfonyl)piperazin-1-yl]-4-[(quinolin-3-ylmethyl)amino]-5,6-dihydro-7H-pyrrolo[3,4-d]pyrimidin-7-one;
(R)-2-(4-acetylpiperazin-1-yl)-4-(1-(6-ethoxy-5-fluoropyridin-3-yl)ethylamino)-6-isopropyl-5H-pyrrolo[3,4-d]pyrimidin-7(6H)-one;
(R)-2-(4-acetylpiperazin-1-yl)-4-(1-(6-ethoxy-5-methylpyridin-3-yl)ethylamino)-6-isopropyl-5H-pyrrolo[3,4-d]pyrimidin-7(6H)-one;
(R)-2-(4-acetylpiperazin-1-yl)-4-(1-(4-(hydroxymethyl)phenyl)ethylamino)-6-isopropyl-5H-pyrrolo[3,4-d]pyrimidin-7(6H)-one;
2-(4-acetylpiperazin-1-yl)-6-isopropyl-4-(2-methyl-1-m-tolylpropan-2-ylamino)-5H-pyrrolo[3,4-d]pyrimidin-7(6H)-one;
2-(4-acetylpiperazin-1-yl)-4-((4-ethoxy-2-methoxybenzyl)(methyl)amino)-6-isopropyl-5H-pyrrolo[3,4-d]pyrimidin-7(6H)-one;
2-(4-acetylpiperazin-1-yl)-4-(4-ethoxy-2-methoxybenzylamino)-6-isopropyl-5H-pyrrolo[3,4-d]pyrimidin-7(6H)-one;
2-(4-acetylpiperazin-1-yl)-4-((2-ethoxy-4-methoxybenzyl)(methyl)amino)-6-isopropyl-5H-pyrrolo[3,4-d]pyrimidin-7(6H)-one;
2-(4-acetylpiperazin-1-yl)-4-(4-isopropoxy-2-methoxybenzylamino)-6-isopropyl-5H-pyrrolo[3,4-d]pyrimidin-7(6H)-one;
2-(4-acetylpiperazin-1-yl)-4-({(1R)-1-[3-fluoro-4-(methoxymethyl)phenyl]ethyl}amino)-6-(1-methylethyl)-5,6-dihydro-7H-pyrrolo[3,4-d]pyrimidin-7-one;
2-(4-acetylpiperazin-1-yl)-4-[{[1-(4-fluorophenyl)-1H-pyrazol-4-yl]methyl}(methyl)amino]-6-(1-methylethyl)-5,6-dihydro-7H-pyrrolo[3,4-d]pyrimidin-7-one;
2-(5,6-dihydroimidazo[1,2-a]pyrazin-7(8H)-yl)-4-{[2-(4-fluorophenyl)-1,1-dimethylethyl]amino}-6-(1-methylethyl)-5,6-dihydro-7H-pyrrolo[3,4-d]pyrimidin-7-one;
2-(4-acetylpiperazin-1-yl)-4-(((7-chloroisoquinolin-3-yl)methyl)(methyl)amino)-6-isopropyl-5H-pyrrolo[3,4-d]pyrimidin-7(6H)-one;
2-(4-acetylpiperazin-1-yl)-6-isopropyl-4-(methyl((6-methylisoquinolin-3-yl)methyl)amino)-5H-pyrrolo[3,4-d]pyrimidin-7(6H)-one;
2-(4-acetylpiperazin-1-yl)-6-isopropyl-4-(quinolin-3-ylmethylamino)-5H-pyrrolo[3,4-d]pyrimidin-7(6H)-one;
(R)-2-(4-acetylpiperazin-1-yl)-6-isopropyl-4-(1-(quinolin-3-yl)ethylamino)-5H-pyrrolo[3,4-d]pyrimidin-7(6H)-one (Enantiomer 1);
(S)-2-(4-acetylpiperazin-1-yl)-6-isopropyl-4-(1-(quinolin-3-yl)ethylamino)-5H-pyrrolo[3,4-d]pyrimidin-7(6H)-one (Enantiomer 2);
2-(4-acetylpiperazin-1-yl)-6-isopropyl-4-(methyl(quinolin-3-ylmethyl)amino)-5H-pyrrolo[3,4-d]pyrimidin-7(6H)-one;
2-(4-acetylpiperazin-1-yl)-6-isopropyl-4-(methyl((2-methylquinolin-3-yl)methyl)amino)-5H-pyrrolo[3,4-d]pyrimidin-7(6H)-one;
2-(4-acetylpiperazin-1-yl)-4-(((6-chloroisoquinolin-3-yl)methyl)(methyl)amino)-6-isopropyl-5H-pyrrolo[3,4-d]pyrimidin-7(6H)-one;
2-(4-acetylpiperazin-1-yl)-4-(((6-fluoroisoquinolin-3-yl)methyl)(methyl)amino)-6-isopropyl-5H-pyrrolo[3,4-d]pyrimidin-7(6H)-one;
2-(4-acetylpiperazin-1-yl)-4-(((7-fluoroisoquinolin-3-yl)methyl)(methyl)amino)-6-isopropyl-5H-pyrrolo[3,4-d]pyrimidin-7(6H)-one;
(R)-4-(1-(2-(4-acetylpiperazin-1-yl)-6-isopropyl-7-oxo-6,7-dihydro-5H-pyrrolo[3,4-d]pyrimidin-4-ylamino)ethyl)benzonitrile;
2-(4-Acetyl-2-methylpiperazin-1-yl)-6-isopropyl-4-(quinolin-3-ylmethylamino)-5H-pyrrolo[3,4-d]pyrimidin-7(6H)-one;
2-(4-acetylpiperazin-1-yl)-6-isopropyl-4-(methyl((7-methylisoquinolin-3-yl)methyl)amino)-5H-pyrrolo[3,4-d]pyrimidin-7(6H)-one;
2-(4-acetylpiperazin-1-yl)-6-(1-methylethyl)-4-{[(1-methyl-1H-indol-5-yl)methyl]amino}-5,6-dihydro-7H-pyrrolo[3,4-d]pyrimidin-7-one;
2-(4-acetylpiperazin-1-yl)-6-(1-methylethyl)-4-{methyl[(1-methyl-1H-indol-6-yl)methyl]amino}-5,6-dihydro-7H-pyrrolo[3,4-d]pyrimidin-7-one;
2-(4-acetylpiperazin-1-yl)-6-(1-methylethyl)-4-{[(1-methyl-1H-indol-6-yl)methyl]amino}-5,6-dihydro-7H-pyrrolo[3,4-d]pyrimidin-7-one;
(R)-2-(4-acetylpiperazin-1-yl)-4-(1-(2,4-diethoxyphenyl)ethylamino)-6-isopropyl-5H-pyrrolo[3,4-d]pyrimidin-7(6H)-one;
(R)-2-(4-acetylpiperazin-1-yl)-4-(1-(4-((dimethylamino)methyl)phenyl)ethylamino)-6-isopropyl-5H-pyrrolo[3,4-d]pyrimidin-7(6H)-one;
(R)-2-(4-acetylpiperazin-1-yl)-4-(1-(2,2-difluorobenzo[d][1,3]dioxol-5-yl)ethylamino)-6-isopropyl-5H-pyrrolo[3,4-d]pyrimidin-7(6H)-one;
2-(4-acetylpiperazin-1-yl)-4-(((2,2-difluorobenzo[d][1,3]dioxol-5-yl)methyl)(methyl)amino)-6-isopropyl-5H-pyrrolo[3,4-d]pyrimidin-7(6H)-one;
(R)-4-(1-(4-ethoxyphenyl)ethylamino)-2-(4-ethyl-3-oxopiperazin-1-yl)-6-isopropyl-5H-pyrrolo[3,4-d]pyrimidin-7(6H)-one;
2-(4-ethyl-3-oxopiperazin-1-yl)-6-isopropyl-4-((isoquinolin-3-ylmethyl)(methyl)amino)-5H-pyrrolo[3,4-d]pyrimidin-7(6H)-one;
(R)-4-(1-(4-ethoxyphenyl)ethylamino)-6-isopropyl-2-(3-oxo-4-(2,2,2-trifluoroethyl)piperazin-1-yl)-5H-pyrrolo[3,4-d]pyrimidin-7(6H)-one;
6-isopropyl-4-((isoquinolin-3-ylmethyl)(methyl)amino)-2-(3-oxo-4-(2,2,2-trifluoroethyl)piperazin-1-yl)-5H-pyrrolo[3,4-d]pyrimidin-7(6H)-one; and pharmaceutically acceptable salts thereof.

17-20. (canceled)

21. A pharmaceutical composition comprising a compound according to claim 1 and a pharmaceutically acceptable carrier.

22. A method for the therapy of pain in a warm-blooded animal, comprising the step of administering to said animal in need of such therapy a therapeutically effective amount of a compound according to claim 1.

23. A method for the therapy of over active bladder in a warm-blooded animal, comprising the step of administering to said animal in need of such therapy a therapeutically effective amount of a compound according to claim 1.

24. A method for preparing a compound of formula 1, 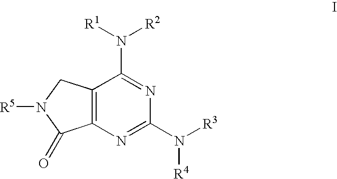
comprising reacting a compound of formula II with HNR³R⁴ 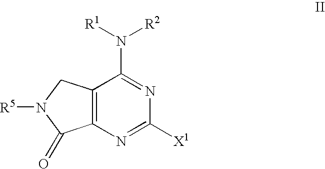
wherein:
X¹ is halogen;
R¹ and R² are independently selected from hydrogen, C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, —NR⁷R⁸, C₃₋₇cycloalkyl fused with a phenyl and C₂₋₆heteroaryl, C₁₋₁₄heterocyclyl, C₁₋₁₄heterocyclyl-C₁₋₆alkyl, C₆₋₁₀aryl, C₆₋₁₀aryl-C₁₋₆alkyl, or R¹ and R² together with the nitrogen connected thereto form a C₂₋₉heterocyclyl; wherein said C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₁₋₁₄heterocyclyl, C₁₋₁₄heterocyclyl-C₁₋₆alkyl, C₆₋₁₀aryl, C₆₋₁₀aryl-C₁₋₆alkyl and C₂₋₉heterocyclyl are optionally substituted by one or more groups selected from, halogen, cyano, nitro, C₁₋₆alkoxy, C₁₋₄haloalkoxy, C₁₋₆alkyl, halogenated C₁₋₆alkyl, C₃₋₆-cycloalkyl, C₃₋₆cycloalkoxy, C₃₋₆cycloalkyl-C₁₋₄alkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—OR⁷, —(CH₂)ₘ—NR⁷R⁸ hydroxy, C₁₋₁₄heterocyclyl-C₀₋₄-alkyl, phenyl, benzyl, phenylethyl, wherein said C₁₋₁₄heterocyclyl-C₀₋₄-alkyl, phenyl, benzyl or phenethyl optionally substituted by one or more groups selected from halogen, cyano, nitro, oxo, C₁₋₆alkoxy, C₁₋₄haloalkoxy, C₁₋₆alkyl, halogenated C₁₋₆alkyl, C₃₋₆-cycloalkyl, C₃₋₆cycloalkoxy, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—OR⁷, —(CH₂)ₘ—NR⁷R⁸ and hydroxy;
R³ and R⁴ are independently selected from hydrogen, C₁₋₆alkyl, C₂₋₆alkenyl, C₃₋₈cycloalkyl, C₃₋₈cycloalkyl-C₁₋₆alkyl, C₆₋₁₀aryl, C₆₋₁₀aryl-C₁₋₆alkyl, C₃₋₆heterocyclyl, and C₃₋₆heterocyclyl-C₁₋₆alkyl; or R³ and R⁴ together with the nitrogen connected thereto form a C₂₋₉heterocyclyl; wherein said C₁₋₆alkyl, C₂₋₆alkenyl, C₃₋₈cycloalkyl, C₃₋₈cycloalkyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl and C₂₋₉heterocyclyl are optionally substituted by one or more groups selected from C₁₋₆alkyl, halogenated C₁₋₆alkyl, carboxy, halogen, cyano, nitro, oxo, C₁₋₄-alkoxy, C₁₋₄haloalkoxy, hydroxy, C₃₋₆cycloalkyl-C₁₋₄alkoxy, C₃₋₆heterocycloalkyl, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—O—C(═O)—R⁷, —(CH₂)ₘ—R⁷, and —(CH₂)ₘ—NR⁷R⁸;
R⁵ is selected from hydrogen and C₁₋₆alkyl, C₃₋₇-cycloalkyl, C₁₋₆heterocyclyl, heteroaryl and C₆₋₁₀aryl, optionally substituted with one or more groups selected from OH, C₁₋₄alkoxy, halogenated C₁₋₄alkoxy, and halogen;
R⁷ and R⁸ are independently selected from —H, C₁₋₆alkyl, C₆₋₁₀aryl, C₁₋₅heterocyclyl, and C₃₋₆cycloalkyl-C₀₋₄alkyl, wherein said C₁₋₆alkyl, C₆₋₁₀aryl, C₁₋₅heterocyclyl, and C₃₋₆cycloalkyl-C₀₋₄alkyl are optionally substituted with one or more groups selected from —OH, methoxy, ethoxy and halogen; and
m is 0, 1, 2 or 3,
with a proviso that at least one of R¹, R², R³ and R⁴ is not hydrogen.

25. A method for preparing a compound of formula II, 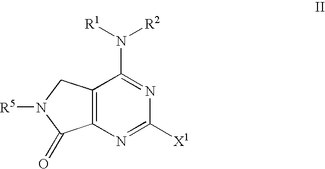
comprising reacting a compound of formula III with HNR¹R² 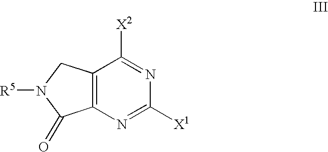
wherein:
X¹ and X² are independently halogens;
R¹ and R² are independently selected from hydrogen, C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₃₋₇cycloalkyl fused with a phenyl and C₂₋₆heteroaryl, C₁₋₁₄heterocyclyl, C₁₋₁₄heterocyclyl-C₁₋₆alkyl, C₆₋₁₀aryl, C₆₋₁₀aryl-C₁₋₆alkyl, or R¹ and R² together with the nitrogen connected thereto form a C₂₋₉heterocyclyl; wherein said C₁₋₆alkyl-C(═O)—, C₁₋₆alkyl, C₂₋₆alkenyl, C₃₋₇cycloalkyl, C₃₋₇cycloalkyl-C₁₋₆alkyl, C₃₋₇cycloalkyl fused with a phenyl, C₁₋₁₄heterocyclyl, C₁₋₁₄heterocyclyl-C₁₋₆alkyl, C₆₋₁₀aryl, C₆₋₁₀aryl-C₁₋₆alkyl and C₂₋₉heterocyclyl are optionally substituted by one or more groups selected from, halogen, cyano, nitro, C₁₋₆alkoxy, C₁₋₄haloalkoxy, C₁₋₆alkyl, halogenated C₁₋₆alkyl, C₃₋₆-cycloalkyl, C₃₋₆cycloalkoxy, C₃₋₆cycloalkyl-C₁₋₄alkoxy, —(CH₂)ₘ—C(═O)NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷—(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—OR⁷, —(CH₂)ₘ—NR⁷R⁸ hydroxy, C₁₋₁₄heterocyclyl-C₀₋₄-alkyl, phenyl, benzyl, phenylethyl, wherein said C₁₋₁₄heterocyclyl-C₀₋₄-alkyl, phenyl, benzyl or phenethyl optionally substituted by one or more groups selected from halogen, cyano, nitro, oxo, C₁₋₆alkoxy, C₁₋₄haloalkoxy, C₁₋₆alkyl, halogenated C₁₋₆alkyl, C₃₋₆-cycloalkyl, C₃₋₆cycloalkoxy, —(CH₂)ₘ—C(═O)NR⁷R⁸, —C(═O)—(CH₂)ₘ—NR⁷R⁸, —(CH₂)ₘ—S(═O)₂NR⁷R⁸, —(CH₂)ₘNH—C(═O)NR⁷R⁸, —(CH₂)ₘ—N(R⁷)C(═O)R⁸, —(CH₂)ₘ—N(R⁷)C(═O)—OR⁸, —(CH₂)ₘ—C(═O)—OR⁷, —(CH₂)ₘ—C(═O)R⁷, —(CH₂)ₘ—S(═O)₂R⁷, —(CH₂)ₘ—S(═O)R⁷, —(CH₂)ₘ—SR⁷, —(CH₂)ₘ—O—C(═O)—R⁷, —(CH₂)ₘ—OR⁷, —(CH₂)ₘ—NR⁷R⁸ and hydroxy;
R⁵ is selected from hydrogen and C₁₋₆alkyl, C₃₋₇-cycloalkyl, C₁₋₆heterocyclyl, heteroaryl and C₆₋₁₀aryl, optionally substituted with one or more groups selected from OH, C₁₋₄alkoxy, halogenated C₁₋₄alkoxy, and halogen;
R⁷ and R⁸ are independently selected from —H, C₁₋₆alkyl, C₆₋₁₀aryl, C₁₋₅heterocyclyl, and C₃₋₆cycloalkyl-C₀₋₄alkyl, wherein said C₁₋₆alkyl, C₆₋₁₀aryl, C₁₋₅heterocyclyl, and C₃₋₆cycloalkyl-C₀₋₄alkyl are optionally substituted with one or more groups selected from —OH, methoxy, ethoxy and halogen; and
m is 0, 1, 2 or 3.

26-28. (canceled)
